SEQUENCE LISTING (25 sequences)

SEQ ID NO: 1 - protein, 1273 aa, Unknown
  Coronaviridae
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - DNA, 30842 bp, Unknown
  ChAdOx2: Viral vector based on Chimpanzee adenovirus C68
ccatcttcaataatatacctcaaactttttgtgcgcgttaatatgcaaatgaggcgtttgaatttggggaggaagggcggtgattggtcgagggatgagcgaccgttaggggcggggcgagtgacgttttgatgacgtggttgcgaggaggagccagtttgcaagttctcgtgggaaaagtgacgtcaaacgaggtgtggtttgaacacggaaatactcaattttcccgcgctctctgacaggaaatgaggtgtttctgggcggatgcaagtgaaaacgggccattttcgcgcgaaaactgaatgaggaagtgaaaatctgagtaatttcgcgtttatggcagggaggagtatttgccgagggccgagtagactttgaccgattacgtgggggtttcgattaccgtgtttttcacctaaatttccgcgtacggtgtcaaagtccggtgtttttacgcgatcgctagcgacatcgatcacaagtttgtacaaaaaagctgaacgagaaacgtaaaatgatataaatatcaatatattaaattagattttgcataaaaaacagactacataatactgtaaaacacaacatatccagtcactatggcggccgccgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgtgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcacgcgtggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggtgatcgattcgacagatcgcgatcgcaagtgagtagtgttctggggcgggggaggacctgcatgagggccagaataactgaaatctgtgcttttctgtgtgttgcagcagcatgagcggaagcggctcctttgagggaggggtattcagcccttatctgacggggcgtctcccctcctgggcgggagtgcgtcagaatgtgatgggatccacggtggacggccggcccgtgcagcccgcgaactcttcaaccctgacctatgcaaccctgagctcttcgtcgttggacgcagctgccgccgcagctgctgcatctgccgccagcgccgtgcgcggaatggccatgggcgccggctactacggcactctggtggccaactcgagttccaccaataatcccgccagcctgaacgaggagaagctgttgctgctgatggcccagctcgaggccttgacccagcgcctgggcgagctgacccagcaggtggctcagctgcaggagcagacgcgggccgcggttgccacggtgaaatccaaataaaaaatgaatcaataaataaacggagacggttgttgattttaacacagagtctgaatctttatttgatttttcgcgcgcggtaggccctggaccaccggtctcgatcattgagcacccggtggatcttttccaggacccggtagaggtgggcttggatgttgaggtacatgggcatgagcccgtcccgggggtggaggtagctccattgcagggcctcgtgctcgggggtggtgttgtaaatcacccagtcatagcaggggcgcagggcatggtgttgcacaatatctttgaggaggagactgatggccacgggcagccctttggtgtaggtgtttacaaatctgttgagctgggagggatgcatgcggggggagatgaggtgcatcttggcctggatcttgagattggcgatgttaccgcccagatcccgcctggggttcatgttgtgcaggaccaccagcacggtgtatccggtgcacttggggaatttatcatgcaacttggaagggaaggcgtgaaagaatttggcgacgcctttgtgcccgcccaggttttccatgcactcatccatgatgatggcgatgggcccgtgggcggcggcctgggcaaagacgtttcgggggtcggacacatcatagttgtggtcctgggtgaggtcatcataggccattttaatgaatttggggcggagggtgccggactgggggacaaaggtaccctcgatcccgggggcgtagttcccctcacagatctgcatctcccaggctttgagctcggagggggggatcatgtccacctgcggggcgataaagaacacggtttccggggcgggggagatgagctgggccgaaagcaagttccggagcagctgggacttgccgcagccggtggggccgtagatgaccccgatgaccggctgcaggtggtagttgagggagagacagctgccgtcctcccggaggaggggggccacctcgttcatcatctcgcgcacgtgcatgttctcgcgcaccagttccgccaggaggcgctctccccccagggataggagctcctggagcgaggcgaagtttttcagcggcttgagtccgtcggccatgggcattttggagagggtttgttgcaagagttccaggcggtcccagagctcggtgatgtgctctacggcatctcgatccagcagacctcctcgtttcgcgggttgggacggctgcgggagtagggcaccagacgatgggcgtccagcgcagccagggtccggtccttccagggtcgcagcgtccgcgtcagggtggtctccgtcacggtgaaggggtgcgcgccgggctgggcgcttgcgagggtgcgcttcaggctcatccggctggtcgaaaaccgctcccgatcggcgccctgcgcgtcggccaggtagcaattgaccatgagttcgtagttgagcgcctcggccgcgtggcctttggcgcggagcttacctttggaagtctgcccgcaggcgggacagaggagggacttgagggcgtagagcttgggggcgaggaagacggactcgggggcgtaggcgtccgcgccgcagtgggcgcagacggtctcgcactccacgagccaggtgaggtcgggctggtcggggtcaaaaaccagtttcccgccgttctttttgatgcgtttcttacctttggtctccatgagctcgtgtccccgctgggtgacaaagaggctgtccgtgtccccgtagaccgactttatgggccggtcctcgagcggtgtgccgcggtcctcctcgtagaggaaccccgcccactccgagacgaaagcccgggtccaggccagcacgaaggaggccacgtgggacgggtagcggtcgttgtccaccagcgggtccaccttttccagggtatgcaaacacatgtccccctcgtccacatccaggaaggtgattggcttgtaagtgtaggccacgtgaccgggggtcccggccgggggggtataaaagggtgcgggtccctgctcgtcctcactgtcttccggatcgctgtccaggagcgccagctgttggggtaggtattccctctcgaaggcgggcatgacctcggcactcaggttgtcagtttctagaaacgaggaggatttgatattgacggtgccggcggagatgcctttcaagagcccctcgtccatctggtcagaaaagacgatctttttgttgtcgagcttggtggcgaaggagccgtagagggcgttggagaggagcttggcgatggagcgcatggtctggtttttttccttgtcggcgcgctccttggcggcgatgttgagctgcacgtactcgcgcgccacgcacttccattcggggaagacggtggtcagctcgtcgggcacgattctgacctgccagccccgattatgcagggtgatgaggtccacactggtggccacctcgccgcgcaggggctcattagtccagcagaggcgtccgcccttgcgcgagcagaaggggggcagggggtccagcatgacctcgtcgggggggtcggcatcgatggtgaagatgccgggcaggaggtcggggtcaaagtagctgatggaagtggccagatcgtccagggcagcttgccattcgcgcacggccagcgcgcgctcgtagggactgaggggcgtgccccagggcatgggatgggtaagcgcggaggcgtacatgccgcagatgtcgtagacgtagaggggctcctcgaggatgccgatgtaggtggggtagcagcgccccccgcggatgctggcgcgcacgtagtcatacagctcgtgcgagggggcgaggagccccgggcccaggttggtgcgactgggcttttcggcgcggtagacgatctggcggaaaatggcatgcgagttggaggagatggtgggcctttggaagatgttgaagtgggcgtggggcagtccgaccgagtcgcggatgaagtgggcgtaggagtcttgcagcttggcgacgagctcggcggtgactaggacgtccagagcgcagtagtcgagggtctcctggatgatgtcatacttgagctgtcccttttgtttccacagctcgcggttgagaaggaactcttcgcggtccttccagtactcttcgagggggaacccgtcctgatctgcacggtaagagcctagcatgtagaactggttgacggccttgtaggcgcagcagcccttctccacggggagggcgtaggcctgggcggccttgcgcagggaggtgtgcgtgagggcgaaagtgtccctgaccatgaccttgaggaactggtgcttgaagtcgatatcgtcgcagcccccctgctcccagagctggaagtccgtgcgcttcttgtaggcggggttgggcaaagcgaaagtaacatcgttgaagaggatcttgcccgcgcggggcataaagttgcgagtgatgcggaaaggttggggcacctcggcccggttgttgatgacctgggcggcgagcacgatctcgtcgaagccgttgatgttgtggcccacgatgtagagttccacgaatcgcggacggcccttgacgtggggcagtttcttgagctcctcgtaggtgagctcgtcggggtcgctgagcccgtgctgctcgagcgcccagtcggcgagatgggggttggcgcggaggaaggaagtccagagatccacggccagggcggtttgcagacggtcccggtactgacggaactgctgcccgacggccattttttcgggggtgacgcagtagaaggtgcgggggtccccgtgccagcgatcccatttgagctggagggcgagatcgagggcgagctcgacgagccggtcgtccccggagagtttcatgaccagcatgaaggggacgagctgcttgccgaaggaccccatccaggtgtaggtttccacatcgtaggtgaggaagagcctttcggtgcgaggatgcgagccgatggggaagaactggatctcctgccaccaattggaggaatggctgttgatgtgatggaagtagaaatgccgacggcgcgccgaacactcgtgcttgtgtttatacaagcggccacagtgctcgcaacgctgcacgggatgcacgtgctgcacgagctgtacctgagttcctttgacgaggaatttcagtgggaagtggagtcgtggcgcctgcatctcgtgctgtactacgtcgtggtggtcggcctggccctcttctgcctcgatggtggtcatgctgacgagcccgcgcgggaggcaggtccagacctcggcgcgagcgggtcggagagcgaggacgagggcgcgcaggccggagctgtccagggtcctgagacgctgcggagtcaggtcagtgggcagcggcggcgcgcggttgacttgcaggagtttttccagggcgcgcgggaggtccagatggtacttgatctccaccgcgccattggtggcgacgtcgatggcttgcagggtcccgtgcccctggggtgtgaccaccgtcccccgtttcttcttgggcggctggggcgacgggggcggtgcctcttccatggttagaagcggcggcgaggacgcgcgccgggcggcaggggcggctcggggcccggaggcaggggcggcaggggcacgtcggcgccgcgcgcgggtaggttctggtactgcgcccggagaagactggcgtgagcgacgacgcgacggttgacgtcctggatctgacgcctctgggtgaaggccacgggacccgtgagtttgaacctgaaagagagttcgacagaatcaatctcggtatcgttgacggcggcctgccgcaggatctcttgcacgtcgcccgagttgtcctggtaggcgatctcggtcatgaactgctcgatctcctcctcttgaaggtctccgcggccggcgcgctccacggtggccgcgaggtcgttggagatgcggcccatgagctgcgagaaggcgttcatgcccgcctcgttccagacgcggctgtagaccacgacgccctcgggatcgccggcgcgcatgaccacctgggcgaggttgagctccacgtggcgcgtgaagaccgcgtagttgcagaggcgctggtagaggtagttgagcgtggtggcgatgtgctcggtgacgaagaaatacatgatccagcggcggagcggcatctcgctgacgtcgcccagcgcctccaaacgttccatggcctcgtaaaagtccacggcgaagttgaaaaactgggagttgcgcgccgagacggtcaactcctcctccagaagacggatgagctcggcgatggtggcgcgcacctcgcgctcgaaggcccccgggagttcctccacttcctcttcttcctcctccactaacatctcttctacttcctcctcaggcggcagtggtggcgggggagggggcctgcgtcgccggcggcgcacgggcagacggtcgatgaagcgctcgatggtctcgccgcgccggcgtcgcatggtctcggtgacggcgcgcccgtcctcgcggggccgcagcgtgaagacgccgccgcgcatctccaggtggccgggggggtccccgttgggcagggagagggcgctgacgatgcatcttatcaattgccccgtagggactccgcgcaaggacctgagcgtctcgagatccacgggatctgaaaaccgctgaacgaaggcttcgagccagtcgcagtcgcaaggtaggctgagcacggtttcttctggcgggtcatgttggttgggagcggggcgggcgatgctgctggtgatgaagttgaaataggcggttctgagacggcggatggtggcgaggagcaccaggtctttgggcccggcttgctggatgcgcagacggtcggccatgccccaggcgtggtcctgacacctggccaggtccttgtagtagtcctgcatgagccgctccacgggcacctcctcctcgcccgcgcggccgtgcatgcgcgtgagcccgaagccgcgctggggctggacgagcgccaggtcggcgacgacgcgctcggcgaggatggcttgctggatctgggtgagggtggtctggaagtcatcaaagtcgacgaagcggtggtaggctccggtgttgatggtgtaggagcagttggccatgacggaccagttgacggtctggtggcccggacgcacgagctcgtggtacttgaggcgcgagtaggcgcgcgtgtcgaagatgtagtcgttgcaggtgcgcaccaggtactggtagccgatgaggaagtgcggcggcggctggcggtagagcggccatcgctcggtggcgggggcgccgggcgcgaggtcctcgagcatggtgcggtggtagccgtagatgtacctggacatccaggtgatgccggcggcggtggtggaggcgcgcgggaactcgcggacgcggttccagatgttgcgcagcggcaggaagtagttcatggtgggcacggtctggcccgtgaggcgcgcgcagtcgtggatgctctatacgggcaaaaacgaaagcggtcagcggctcgactccgtggcctggaggctaagcgaacgggttgggctgcgcgtgtaccccggttcgaatctcgaatcaggctggagccgcagctaacgtggtattggcactcccgtctcgacccaagcctgcaccaaccctccaggatacggaggcgggtcgttttgcaacttttttttggaggccggatgagactagtaagcgcggaaagcggccgaccgcgatggctcgctgccgtagtctggagaagaatcgccagggttgcgttgcggtgtgccccggttcgaggccggccggattccgcggctaacgagggcgtggctgccccgtcgtttccaagaccccatagccagccgacttctccagttacggagcgagcccctcttttgttttgtttgtttttgccagatgcatcccgtactgcggcagatgcgcccccaccaccctccaccgcaacaacagccccctccacagccggcgcttctgcccccgccccagcagcaacttccagccacgaccgccgcggccgccgtgagcggggctggacagagttatgatcaccagctggccttggaagagggcgaggggctggcgcgcctgggggcgtcgtcgccggagcggcacccgcgcgtgcagatgaaaagggacgctcgcgaggcctacgtgcccaagcagaacctgttcagagacaggagcggcgaggagcccgaggagatgcgcgcggcccggttccacgcggggcgggagctgcggcgcggcctggaccgaaagagggtgctgagggacgaggatttcgaggcggacgagctgacggggatcagccccgcgcgcgcgcacgtggccgcggccaacctggtcacggcgtacgagcagaccgtgaaggaggagagcaacttccaaaaatccttcaacaaccacgtgcgcaccctgatcgcgcgcgaggaggtgaccctgggcctgatgcacctgtgggacctgctggaggccatcgtgcagaaccccaccagcaagccgctgacggcgcagctgttcctggtggtgcagcatagtcgggacaacgaagcgttcagggaggcgctgctgaatatcaccgagcccgagggccgctggctcctggacctggtgaacattctgcagagcatcgtggtgcaggagcgcgggctgccgctgtccgagaagctggcggccatcaacttctcggtgctgagtttgggcaagtactacgctaggaagatctacaagaccccgtacgtgcccatagacaaggaggtgaagatcgacgggttttacatgcgcatgaccctgaaagtgctgaccctgagcgacgatctgggggtgtaccgcaacgacaggatgcaccgtgcggtgagcgccagcaggcggcgcgagctgagcgaccaggagctgatgcatagtctgcagcgggccctgaccggggccgggaccgagggggagagctactttgacatgggcgcggacctgcactggcagcccagccgccgggccttggaggcggcggcaggaccctacgtagaagaggtggacgatgaggtggacgaggagggcgagtacctggaagactgatggcgcgaccgtatttttgctagatgcaacaacaacagccacctcctgatcccgcgatgcgggcggcgctgcagagccagccgtccggcattaactcctcggacgattggacccaggccatgcaacgcatcatggcgctgacgacccgcaaccccgaagcctttagacagcagccccaggccaaccggctctcggccatcctggaggccgtggtgccctcgcgctccaaccccacgcacgagaaggtcctggccatcgtgaacgcgctggtggagaacaaggccatccgcggcgacgaggccggcctggtgtacaacgcgctgctggagcgcgtggcccgctacaacagcaccaacgtgcagaccaacctggaccgcatggtgaccgacgtgcgcgaggccgtggcccagcgcgagcggttccaccgcgagtccaacctgggatccatggtggcgctgaacgccttcctcagcacccagcccgccaacgtgccccggggccaggaggactacaccaacttcatcagcgccctgcgcctgatggtgaccgaggtgccccagagcgaggtgtaccagtccgggccggactacttcttccagaccagtcgccagggcttgcagaccgtgaacctgagccaggctttcaagaacttgcagggcctgtggggcgtgcaggccccggtcggggaccgcgcgacggtgtcgagcctgctgacgccgaactcgcgcctgctgctgctgctggtggcccccttcacggacagcggcagcatcaaccgcaactcgtacctgggctacctgattaacctgtaccgcgaggccatcggccaggcgcacgtggacgagcagacctaccaggagatcacccacgtgagccgcgccctgggccaggacgacccgggcaacctggaagccaccctgaactttttgctgaccaaccggtcgcagaagatcccgccccagtacgcgctcagcaccgaggaggagcgcatcctgcgttacgtgcagcagagcgtgggcctgttcctgatgcaggagggggccacccccagcgccgcgctcgacatgaccgcgcgcaacatggagcccagcatgtacgccagcaaccgcccgttcatcaataaactgatggactacttgcatcgggcggccgccatgaactctgactatttcaccaacgccatcctgaatccccactggctcccgccgccggggttctacacgggcgagtacgacatgcccgaccccaatgacgggttcctgtgggacgatgtggacagcagcgtgttctccccccgaccgggtgctaacgagcgccccttgtggaagaaggaaggcagcgaccgacgcccgtcctcggcgctgtccggccgcgagggtgctgccgcggcggtgcccgaggccgccagtcctttcccgagcttgcccttctcgctgaacagtatccgcagcagcgagctgggcaggatcacgcgcccgcgcttgctgggcgaagaggagtacttgaatgactcgctgttgagacccgagcgggagaagaacttccccaataacgggatagaaagcctggtggacaagatgagccgctggaagacgtatgcgcaggagcacagggacgatccccgggcgtcgcagggggccacgagccggggcagcgccgcccgtaaacgccggtggcacgacaggcagcggggacagatgtgggacgatgaggactccgccgacgacagcagcgtgttggacttgggtgggagtggtaacccgttcgctcacctgcgcccccgtatcgggcgcatgatgtaagagaaaccgaaaataaatgatactcaccaaggccatggcgaccagcgtgcgttcgtttcttctctgttgttgttgtatctagtatgatgaggcgtgcgtacccggagggtcctcctccctcgtacgagagcgtgatgcagcaggcgatggcggcggcggcgatgcagcccccgctggaggctccttacgtgcccccgcggtacctggcgcctacggaggggcggaacagcattcgttactcggagctggcacccttgtacgataccacccggttgtacctggtggacaacaagtcggcggacatcgcctcgctgaactaccagaacgaccacagcaacttcctgaccaccgtggtgcagaacaatgacttcacccccacggaggccagcacccagaccatcaactttgacgagcgctcgcggtggggcggccagctgaaaaccatcatgcacaccaacatgcccaacgtgaacgagttcatgtacagcaacaagttcaaggcgcgggtgatggtctcccgcaagacccccaatggggtgacagtgacagaggattatgatggtagtcaggatgagctgaagtatgaatgggtggaatttgagctgcccgaaggcaacttctcggtgaccatgaccatcgacctgatgaacaacgccatcatcgacaattacttggcggtggggcggcagaacggggtgctggagagcgacatcggcgtgaagttcgacactaggaacttcaggctgggctgggaccccgtgaccgagctggtcatgcccggggtgtacaccaacgaggctttccatcccgatattgtcttgctgcccggctgcggggtggacttcaccgagagccgcctcagcaacctgctgggcattcgcaagaggcagcccttccaggaaggcttccagatcatgtacgaggatctggaggggggcaacatccccgcgctcctggatgtcgacgcctatgagaaaagcaaggaggatgcagcagctgaagcaactgcagccgtagctaccgcctctaccgaggtcaggggcgataattttgcaagcgccgcagcagtggcagcggccgaggcggctgaaaccgaaagtaagatagtcattcagccggtggagaaggatagcaagaacaggagctacaacgtactaccggacaagataaacaccgcctaccgcagctggtacctagcctacaactatggcgaccccgagaagggcgtgcgctcctggacgctgctcaccacctcggacgtcacctgcggcgtggagcaagtctactggtcgctgcccgacatgatgcaagacccggtcaccttccgctccacgcgtcaagttagcaactacccggtggtgggcgccgagctcctgcccgtctactccaagagcttcttcaacgagcaggccgtctactcgcagcagctgcgcgccttcacctcgcttacgcacgtcttcaaccgcttccccgagaaccagatcctcgtccgcccgcccgcgcccaccattaccaccgtcagtgaaaacgttcctgctctcacagatcacgggaccctgccgctgcgcagcagtatccggggagtccagcgcgtgaccgttactgacgccagacgccgcacctgcccctacgtctacaaggccctgggcatagtcgcgccgcgcgtcctctcgagccgcaccttctaaatgtccattctcatctcgcccagtaataacaccggttggggcctgcgcgcgcccagcaagatgtacggaggcgctcgccaacgctccacgcaacaccccgtgcgcgtgcgcgggcacttccgcgctccctggggcgccctcaagggccgcgtgcggtcgcgcaccaccgtcgacgacgtgatcgaccaggtggtggccgacgcgcgcaactacacccccgccgccgcgcccgtctccaccgtggacgccgtcatcgacagcgtggtggcggacgcgcgccggtacgcccgcgccaagagccggcggcggcgcatcgcccggcggcaccggagcacccccgccatgcgcgcggcgcgagccttgctgcgcagggccaggcgcacgggacgcagggccatgctcagggcggccagacgcgcggcttcaggcgccagcgccggcaggacccggagacgcgcggccacggcggcggcagcggccatcgccagcatgtcccgcccgcggcgagggaacgtgtactgggtgcgcgacgccgccaccggtgtgcgcgtgcccgtgcgcacccgcccccctcgcacttgaagatgttcacttcgcgatgttgatgtgtcccagcggcgaggaggatgtccaagcgcaaattcaaggaagagatgctccaggtcatcgcgcctgagatctacggccctgcggtggtgaaggaggaaagaaagccccgcaaaatcaagcgggtcaaaaaggacaaaaaggaagaagaaagtgatgtggacggattggtggagtttgtgcgcgagttcgccccccggcggcgcgtgcagtggcgcgggcggaaggtgcaaccggtgctgagacccggcaccaccgtggtcttcacgcccggcgagcgctccggcaccgcttccaagcgctcctacgacgaggtgtacggggatgatgatattctggagcaggcggccgagcgcctgggcgagtttgcttacggcaagcgcagccgttccgcaccgaaggaagaggcggtgtccatcccgctggaccacggcaaccccacgccgagcctcaagcccgtgaccttgcagcaggtgctgccgaccgcggcgccgcgccgggggttcaagcgcgagggcgaggatctgtaccccaccatgcagctgatggtgcccaagcgccagaagctggaagacgtgctggagaccatgaaggtggacccggacgtgcagcccgaggtcaaggtgcggcccatcaagcaggtggccccgggcctgggcgtgcagaccgtggacatcaagattcccacggagcccatggaaacgcagaccgagcccatgatcaagcccagcaccagcaccatggaggtgcagacggatccctggatgccatcggctcctagtcgaagaccccggcgcaagtacggcgcggccagcctgctgatgcccaactacgcgctgcatccttccatcatccccacgccgggctaccgcggcacgcgcttctaccgcggtcataccagcagccgccgccgcaagaccaccactcgccgccgccgtcgccgcaccgccgctgcaaccacccctgccgccctggtgcggagagtgtaccgccgcggccgcgcacctctgaccctgccgcgcgcgcgctaccacccgagcatcgccatttaaactttcgccagctttgcagatcaatggccctcacatgccgccttcgcgttcccattacgggctaccgaggaagaaaaccgcgccgtagaaggctggcggggaacgggatgcgtcgccaccaccaccggcggcggcgcgccatcagcaagcggttggggggaggcttcctgcccgcgctgatccccatcatcgccgcggcgatcggggcgatccccggcattgcttccgtggcggtgcaggcctctcagcgccactgagacacacttggaaacatcttgtaataaacccatggactctgacgctcctggtcctgtgatgtgttttcgtagacagatggaagacatcaatttttcgtccctggctccgcgacacggcacgcggccgttcatgggcacctggagcgacatcggcaccagccaactgaacgggggcgccttcaattggagcagtctctggagcgggcttaagaatttcgggtccacgcttaaaacctatggcagcaaggcgtggaacagcaccacagggcaggcgctgagggataagctgaaagagcagaacttccagcagaaggtggtcgatgggctcgcctcgggcatcaacggggtggtggacctggccaaccaggccgtgcagcggcagatcaacagccgcctggacccggtgccgcccgccggctccgtggagatgccgcaggtggaggaggagctgcctcccctggacaagcggggcgagaagcgaccccgccccgatgcggaggagacgctgctgacgcacacggacgagccgcccccgtacgaggaggcggtgaaactgggtctgcccaccacgcggcccatcgcgcccctggccaccggggtgctgaaacccgaaaagcccgcgaccctggacttgcctcctccccagccttcccgcccctctacagtggctaagcccctgccgccggtggccgtggcccgcgcgcgacccgggggcaccgcccgccctcatgcgaactggcagagcactctgaacagcatcgtgggtctgggagtgcagagtgtgaagcgccgccgctgctattaaacctaccgtagcgcttaacttgcttgtctgtgtgtgtatgtattatgtcgccgccgccgctgtccaccagaaggaggagtgaagaggcgcgtcgccgagttgcaagatggccaccccatcgatgctgccccagtgggcgtacatgcacatcgccggacaggacgcttcggagtacctgagtccgggtctggtgcagtttgcccgcgccacagacacctacttcagtctggggaacaagtttaggaaccccacggtggcgcccacgcacgatgtgaccaccgaccgcagccagcggctgacgctgcgcttcgtgcccgtggaccgcgaggacaacacctactcgtacaaagtgcgctacacgctggccgtgggcgacaaccgcgtgctggacatggccagcacctactttgacatccgcggcgtgctggatcggggccctagcttcaaaccctactccggcaccgcctacaacagtctggcccccaagggagcacccaacacttgtcagtggacatataaagccgatggtgaaactgccacagaaaaaacctatacatatggaaatgcacccgtgcagggcattaacatcacaaaagatggtattcaacttggaactgacaccgatgatcagccaatctacgcagataaaacctatcagcctgaacctcaagtgggtgatgctgaatggcatgacatcactggtactgatgaaaagtatggaggcagagctcttaagcctgataccaaaatgaagccttgttatggttcttttgccaagcctactaataaagaaggaggtcaggcaaatgtgaaaacaggaacaggcactactaaagaatatgacatagacatggctttctttgacaacagaagtgcggctgctgctggcctagctccagaaattgttttgtatactgaaaatgtggatttggaaactccagatacccatattgtatacaaagcaggcacagatgacagcagctcttctattaatttgggtcagcaagccatgcccaacagacctaactacattggtttcagagacaactttatcgggctcatgtactacaacagcactggcaatatgggggtgctggccggtcaggcttctcagctgaatgctgtggttgacttgcaagacagaaacaccgagctgtcctaccagctcttgcttgactctctgggtgacagaacccggtatttcagtatgtggaatcaggcggtggacagctatgatcctgatgtgcgcattattgaaaatcatggtgtggaggatgaacttcccaactattgtttccctctggatgctgttggcagaacagatacttatcagggaattaaggctaatggaactgatcaaaccacatggaccaaagatgacagtgtcaatgatgctaatgagataggcaagggtaatccattcgccatggaaatcaacatccaagccaacctgtggaggaacttcctctacgccaacgtggccctgtacctgcccgactcttacaagtacacgccggccaatgttaccctgcccaccaacaccaacacctacgattacatgaacggccgggtggtggcgccctcgctggtggactcctacatcaacatcggggcgcgctggtcgctggatcccatggacaacgtgaaccccttcaaccaccaccgcaatgcggggctgcgctaccgctccatgctcctgggcaacgggcgctacgtgcccttccacatccaggtgccccagaaatttttcgccatcaagagcctcctgctcctgcccgggtcctacacctacgagtggaacttccgcaaggacgtcaacatgatcctgcagagctccctcggcaacgacctgcgcacggacggggcctccatctccttcaccagcatcaacctctacgccaccttcttccccatggcgcacaacacggcctccacgctcgaggccatgctgcgcaacgacaccaacgaccagtccttcaacgactacctctcggcggccaacatgctctaccccatcccggccaacgccaccaacgtgcccatctccatcccctcgcgcaactgggccgccttccgcggctggtccttcacgcgtctcaagaccaaggagacgccctcgctgggctccgggttcgacccctacttcgtctactcgggctccatcccctacctcgacggcaccttctacctcaaccacaccttcaagaaggtctccatcaccttcgactcctccgtcagctggcccggcaacgaccggctcctgacgcccaacgagttcgaaatcaagcgcaccgtcgacggcgagggctacaacgtggcccagtgcaacatgaccaaggactggttcctggtccagatgctggcccactacaacatcggctaccagggcttctacgtgcccgagggctacaaggaccgcatgtactccttcttccgcaacttccagcccatgagccgccaggtggtggacgaggtcaactacaaggactaccaggccgtcaccctggcctaccagcacaacaactcgggcttcgtcggctacctcgcgcccaccatgcgccagggccagccctaccccgccaactacccctacccgctcatcggcaagagcgccgtcaccagcgtcacccagaaaaagttcctctgcgacagggtcatgtggcgcatccccttctccagcaacttcatgtccatgggcgcgctcaccgacctcggccagaacatgctctatgccaactccgcccacgcgctagacatgaatttcgaagtcgaccccatggatgagtccacccttctctatgttgtcttcgaagtcttcgacgtcgtccgagtgcaccagccccaccgcggcgtcatcgaggccgtctacctgcgcacccccttctcggccggtaacgccaccacctaagctcttgcttcttgcaagccatggccgcgggctccggcgagcaggagctcagggccatcatccgcgacctgggctgcgggccctacttcctgggcaccttcgataagcgcttcccgggattcatggccccgcacaagctggcctgcgccatcgtcaacacggccggccgcgagaccgggggcgagcactggctggccttcgcctggaacccgcgctcgaacacctgctacctcttcgaccccttcgggttctcggacgagcgcctcaagcagatctaccagttcgagtacgagggcctgctgcgccgcagcgccctggccaccgaggaccgctgcgtcaccctggaaaagtccacccagaccgtgcagggtccgcgctcggccgcctgcgggctcttctgctgcatgttcctgcacgccttcgtgcactggcccgaccgccccatggacaagaaccccaccatgaacttgctgacgggggtgcccaacggcatgctccagtcgccccaggtggaacccaccctgcgccgcaaccaggaggcgctctaccgcttcctcaactcccactccgcctactttcgctcccaccgcgcgcgcatcgagaaggccaccgccttcgaccgcatgaatcaagacatgtaaaccgtgtgtgtatgttaaatgtctttaataaacagcactttcatgttacacatgcatctgagatgatttatttagaaatcgaaagggttctgccgggtctcggcatggcccgcgggcagggacacgttgcggaactggtacttggccagccacttgaactcggggatcagcagtttgggcagcggggtgtcggggaaggagtcggtccacagcttccgcgtcagttgcagggcgcccagcaggtcgggcgcggagatcttgaaatcgcagttgggacccgcgttctgcgcgcgggagttgcggtacacggggttgcagcactggaacaccatcagggccgggtgcttcacgctcgccagcaccgtcgcgtcggtgatgctctccacgtcgaggtcctcggcgttggccatcccgaagggggtcatcttgcaggtctgccttcccatggtgggcacgcacccgggcttgtggttgcaatcgcagtgcagggggatcagcatcatctgggcctggtcggcgttcatccccgggtacatggccttcatgaaagcctccaattgcctgaacgcctgctgggccttggctccctcggtgaagaagaccccgcaggacttgctagagaactggttggtggcgcacccggcgtcgtgcacgcagcagcgcgcgtcgttgttggccagctgcaccacgctgcgcccccagcggttctgggtgatcttggcccggtcggggttctccttcagcgcgcgctgcccgttctcgctcgccacatccatctcgatcatgtgctccttctggatcatggtggtcccgtgcaggcaccgcagcttgccctcggcctcggtgcacccgtgcagccacagcgcgcacccggtgcactcccagttcttgtgggcgatctgggaatgcgcgtgcacgaagccctgcaggaagcggcccatcatggtggtcagggtcttgttgctagtgaaggtcagcggaatgccgcggtgctcctcgttgatgtacaggtggcagatgcggcggtacacctcgccctgctcgggcatcagctggaagttggctttcaggtcggtctccacgcggtagcggtccatcagcatagtcatgatttccatacccttctcccaggccgagacgatgggcaggctcatagggttcttcaccatcatcttagcgctagcagccgcggccagggggtcgctctcgtccagggtctcaaagctccgcttgccgtccttctcggtgatccgcaccggggggtagctgaagcccacggccgccagctcctcctcggcctgtctttcgtcctcgctgtcctggctgacgtcctgcaggaccacatgcttggtcttgcggggtttcttcttgggcggcagcggcggcggagatgttggagatggcgagggggagcgcgagttctcgctcaccactactatctcttcctcttcttggtccgaggccacgcggcggtaggtatgtctcttcgggggcagaggcggaggcgacgggctctcgccgccgcgacttggcggatggctggcagagccccttccgcgttcgggggtgcgctcccggcggcgctctgactgacttcctccgcggccggccattgtgttctcctagggaggaacaacaagcatggagactcagccatcgccaacctcgccatctgcccccaccgccgacgagaagcagcagcagcagaatgaaagcttaaccgccccgccgcccagccccgccacctccgacgcggccgtcccagacatgcaagagatggaggaatccatcgagattgacctgggctatgtgacgcccgcggagcacgaggaggagctggcagtgcgcttttcacaagaagagatacaccaagaacagccagagcaggaagcagagaatgagcagagtcaggctgggctcgagcatgacggcgactacctccacctgagcgggggggaggacgcgctcatcaagcatctggcccggcaggccaccatcgtcaaggatgcgctgctcgaccgcaccgaggtgcccctcagcgtggaggagctcagccgcgcctacgagttgaacctcttctcgccgcgcgtgccccccaagcgccagcccaatggcacctgcgagcccaacccgcgcctcaacttctacccggtcttcgcggtgcccgaggccctggccacctaccacatctttttcaagaaccaaaagatccccgtctcctgccgcgccaaccgcacccgcgccgacgcccttttcaacctgggtcccggcgcccgcctacctgatatcgcctccttggaagaggttcccaagatcttcgagggtctgggcagcgacgagactcgggccgcgaacgctctgcaaggagaaggaggagagcatgagcaccacagcgccctggtcgagttggaaggcgacaacgcgcggctggcggtgctcaaacgcacggtcgagctgacccatttcgcctacccggctctgaacctgccccccaaagtcatgagcgcggtcatggaccaggtgctcatcaagcgcgcgtcgcccatctccgaggacgagggcatgcaagactccgaggagggcaagcccgtggtcagcgacgagcagctggcccggtggctgggtcctaatgctagtccccagagtttggaagagcggcgcaaactcatgatggccgtggtcctggtgaccgtggagctggagtgcctgcgccgcttcttcgccgacgcggagaccctgcgcaaggtcgaggagaacctgcactacctcttcaggcacgggttcgtgcgccaggcctgcaagatctccaacgtggagctgaccaacctggtctcctacatgggcatcttgcacgagaaccgcctggggcagaacgtgctgcacaccaccctgcgcggggaggcccggcgcgactacatccgcgactgcgtctacctctacctctgccacacctggcagacgggcatgggcgtgtggcagcagtgtctggaggagcagaacctgaaagagctctgcaagctcctgcagaagaacctcaagggtctgtggaccgggttcgacgagcgcaccaccgcctcggacctggccgacctcattttccccgagcgcctcaggctgacgctgcgcaacggcctgcccgactttatgagccaaagcatgttgcaaaactttcgctctttcatcctcgaacgctccggaatcctgcccgccacctgctccgcgctgccctcggacttcgtgccgctgaccttccgcgagtgccccccgccgctgtggagccactgctacctgctgcgcctggccaactacctggcctaccactcggacgtgatcgaggacgtcagcggcgagggcctgctcgagtgccactgccgctgcaacctctgcacgccgcaccgctccctggcctgcaacccccagctgctgagcgagacccagatcatcggcaccttcgagttgcaagggcccagcgaaggcgagggttcagccgccaaggggggtctgaaactcaccccggggctgtggacctcggcctacttgcgcaagttcgtgcccgaggactaccatcccttcgagatcaggttctacgaggaccaatcccatccgcccaaggccgagctgtcggcctgcgtcatcacccagggggcgatcctggcccaattgcaagccatccagaaatcccgccaagaattcttgctgaaaaagggccgcggggtctacctcgacccccagaccggtgaggagctcaaccccggcttcccccaggatgccccgaggaaacaagaagctgaaagtggagctgccgcccgtggaggatttggaggaagactgggagaacagcagtcaggcagaggaggaggagatggaggaagactgggacagcactcaggcagaggaggacagcctgcaagacagtctggaggaagacgaggaggaggcagaggaggaggtggaagaagcagccgccgccagaccgtcgtcctcggcgggggagaaagcaagcagcacggataccatctccgctccgggtcggggtcccgctcgaccacacagtagatgggacgagaccggacgattcccgaaccccaccacccagaccggtaagaaggagcggcagggatacaagtcctggcgggggcacaaaaacgccatcgtctcctgcttgcaggcctgcgggggcaacatctccttcacccggcgctacctgctcttccaccgcggggtgaactttccccgcaacatcttgcattactaccgtcacctccacagcccctactacttccaagaagaggcagcagcagcagaaaaagaccagcagaaaaccagcagctagaaaatccacagcggcggcagcaggtggactgaggatcgcggcgaacgagccggcgcaaacccgggagctgaggaaccggatctttcccaccctctatgccatcttccagcagagtcgggggcaggagcaggaactgaaagtcaagaaccgttctctgcgctcgctcacccgcagttgtctgtatcacaagagcgaagaccaacttcagcgcactctcgaggacgccgaggctctcttcaacaagtactgcgcgctcactcttaaagagtagcccgcgcccgcccagtcgcagaaaaaggcgggaattacgtcacctgtgcccttcgccctagccgcctccacccatcatcatgagcaaagagattcccacgccttacatgtggagctaccagccccagatgggcctggccgccggtgccgcccaggactactccacccgcatgaattggctcagcgccgggcccgcgatgatctcacgggtgaatgacatccgcgcccaccgaaaccagatactcctagaacagtcagcgctcaccgccacgccccgcaatcacctcaatccgcgtaattggcccgccgccctggtgtaccaggaaattccccagcccacgaccgtactacttccgcgagacgcccaggccgaagtccagctgactaactcaggtgtccagctggcgggcggcgccaccctgtgtcgtcaccgccccgctcagggtataaagcggctggtgatccggggcagaggcacacagctcaacgacgaggtggtgagctcttcgctgggtctgcgacctgacggagtcttccaactcgccggatcggggagatcttccttcacgcctcgtcaggccgtcctgactttggagagttcgtcctcgcagccccgctcgggtggcatcggcactctccagttcgtggaggagttcactccctcggtctacttcaaccccttctccggctcccccggccactacccggacgagttcatcccgaacttcgacgccatcagcgagtcggtggacggctacgattgagtttaaactcacccccttatccagtgaaataaagatcatattgatgatgattttacagaaataaaaaataatcatttgatttgaaataaagatacaatcatattgatgatttgagtttaacaaaaaaataaagaatcacttacttgaaatctgataccaggtctctgtccatgttttctgccaacaccacttcactcccctcttcccagctctggtactgcaggccccggcgggctgcaaacttcctccacacgctgaaggggatgtcaaattcctcctgtccctcaatcttcattttatcttctatcagatgtccaaaaagcgcgtccgggtggatgatgacttcgaccccgtctacccctacgatgcagacaacgcaccgaccgtgcccttcatcaacccccccttcgtctcttcagatggattccaagagaagcccctgggggtgttgtccctgcgactggccgaccccgtcaccaccaagaacggggaaatcaccctcaagctgggagagggggtggacctcgattcctcgggaaaactcatctccaacacggccaccaaggccgccgcccctctcagtttttccaacaacaccatttcccttaacatggatcaccccttttacactaaagatggaaaattatccttacaagtttctccaccattaaatatactgagaacaagcattctaaacacactagctttaggttttggatcaggtttaggactccgtggctctgccttggcagtacagttagtctctccacttacatttgatactgatggaaacataaagcttaccttagacagaggtttgcatgttacaacaggagatgcaattgaaagcaacataagctgggctaaaggtttaaaatttgaagatggagccatagcaaccaacattggaaatgggttagagtttggaagcagtagtacagaaacaggtgttgatgatgcttacccaatccaagttaaacttggatctggccttagctttgacagtacaggagccataatggctggtaacaaagaagacgataaactcactttgtggacaacacctgatccatcaccaaactgtcaaatactcgcagaaaatgatgcaaaactaacactttgcttgactaaatgtggtagtcaaatactggccactgtgtcagtcttagttgtaggaagtggaaacctaaaccccattactggcaccgtaagcagtgctcaggtgtttctacgttttgatgcaaacggtgttcttttaacagaacattctacactaaaaaaatactgggggtataggcagggagatagcatagatggcactccatataccaatgctgtaggattcatgcccaatttaaaagcttatccaaagtcacaaagttctactactaaaaataatatagtagggcaagtatacatgaatggagatgtttcaaaacctatgcttctcactataaccctcaatggtactgatgacagcaacagtacatattcaatgtcattttcatacacctggactaatggaagctatgttggagcaacatttggggctaactcttataccttctcatacatcgcccaagaatgaacactgtatcccaccctgcatgccaacccttcccaccccactctgtggaacaaactctgaaacacaaaataaaataaagttcaagtgttttattgattcaacagtttcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgattaactttattccaaacggtctcggagcacttcaaaatgcaggtcccggaggtggcacctctcgcccccactgtgttggtggaaaataacagccaggtcaaaggtgacacggttctcgagatgttccacggtggcttccagcaaagcctccacgcgcacatccagaaacaagaggacagcgaaagcgggagcgttttctaattcctcaatcatcatattacactcctgcaccatccccagataattttcatttttccagccttgaatgattcgtattagttcctgaggtaaatccaagccagccatgataaaaagctcgcgcagagcgccctccaccggcattcttaagcacaccctcataattccaagagattctgctcctggttcacctgcagcagattaacaatgggaatatcaaaatctctgccgcgatccctaagctcctccctcaacaataactgtatgtaatctttcatatcatctccgaaatttttagccatagggccgccaggaataagagcagggcaagccacattacagataaagcgaagtcctccccagtgwgcattgccaaatgtaagattgaaataagcatgctggctagaccctgtgatatcttccagataactggacagaaaatcaggcaagcaatttttaagaaaatcaacaaaagaaaagtcgtccaggtgcaggtttagagcctcaggaacaacgatggaataagtgcaaggagtgcgttccagcatggttagtgtttttttggtgatctgtagaacaaaaaataaacatgcaatattaaaccatgctagcctggcgaacaggtgggtaaatcactctttccagcaccaggcaggctacggggtctccggcgcgaccctcgtagaagctgtcgccatgattgaaaagcatcaccgagagaccttcccggtggccggcatggatgattcgagaagaagcatacactccgggaacattggcatccgtgagtgaaaaaaagcgacctataaagcctcggggcactacaatgctcaatctcaattccagcaaagccaccccatgcggatggagcacaaaattggcaggtgcgtaaaaaatgtaattactcccctcctgcacaggcagcaaagcccccgctccctccagaaacacatacaaagcctcagcgtccatagcttaccgagcacggcaggcgcaagagtcagagaaaaggctgagctctaacctgactgcccgctcctgtgctcaatatatagccctaacctacactgacgtaaaggccaaagtctaaaaatacccgccaaataatcacacacgcccagcacacgcccagaaaccggtgacacactcaaaaaaatacgcgcacttcctcaaacgcccaaaactgccgtcatttccgggttcccacgctacgtcatcaaaacacgactttcaaattccgtcgaccgttaaaaacgtcacccgccccgcccctaacggtcgcccgtctctcagccaatcagcgccccgcatccccaaattcaaacacctcatttgcatattaacgcgcacaaaaagtttgaggtatattattgatgatgg

SEQ ID NO: 3 - protein, 1301 aa, Unknown
  amino acid sequence of tPA - 2816 Spike Protein fusion (tPA underlined) (AZD2816)
MDAMKRGLCCVLLLCGAVFVSASQEIHARFRRFVFLVLLPLVSSQCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 4 - DNA, 3915 bp, Unknown
  AZD1222 codon optimised with tPA leader
atggacgccatgaagcgaggcctgtgctgtgttctgcttctgtgtggcgccgtgtttgtgtccgccagccaagagatccacgccagatttcggagattcgtgttcctggtgctgctgccactggtgtctagccagtgtgtgaacctgaccaccaggacacagctgcctccagcctacaccaacagcttcaccagaggcgtgtactacccagacaaggtgttcagatccagcgtgctgcactctacccaggacctgttcctgcctttcttcagcaacgtgacctggttccacgccatccacgtgtccggcaccaatggcaccaagagattcgacaacccagtgctgcccttcaacgacggagtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgacccattcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacacctatcaacctcgtgcgggatctgcctcagggcttctctgctctggaaccactggtggatctgcccatcggcatcaacatcacccggtttcagacactgctggccctgcacagaagctacctgacacctggcgatagcagctctggatggacagctggcgccgctgcctactatgtgggatacctgcagcctcggaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttcccgaatatcaccaatctgtgcccattcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcacctaccaagctgaacgacctgtgcttcacaaacgtgtacgccgacagcttcgtgatccgtggagatgaagtgcggcagattgcacctggacagacaggcaagatcgccgactacaactacaagctgcccgacgacttcaccggctgtgtgattgcctggaacagcaacaacctggatagcaaagtcggcggcaactacaattacctgtaccggctgttccggaagtccaatctgaagcccttcgagcgggacatctccaccgagatctatcaggccggcagcacaccttgtaacggcgtggaaggcttcaactgctacttcccactgcagtcctacggctttcagcccacaaatggcgtgggctaccagccttacagagtggtggtgctgagcttcgagctgctgcatgctcctgccacagtgtgcggccctaagaaaagcaccaatctcgtgaagaacaaatgcgtgaacttcaacttcaacggcctgaccggcaccggcgtgctgacagagagcaacaagaagttcctgccattccagcagttcggccgggatatcgccgataccacagatgccgtcagagatcctcagacactggaaatcctggacatcacaccttgcagcttcggcggagtgtctgtgatcacgcctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccctatcggcgctggcatctgcgcctcttaccagacacagacaaacagccctagacgggccagatctgtggccagccagagcatcattgcctacacaatgtctctgggcgccgagaacagcgtggcctactccaacaactctatcgctatcccgaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacgcctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggcgctgccctgcagatcccatttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtctagcaacttcggcgccatcagctctgtgctgaacgatatcctgagcagactggacaaggtggaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcgcctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagcctcagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccctgacgtggacctgggcgatatcagcggaatcaatgccagcgtcgtgaacatccagaaagagatcgaccggctgaacgaggtggccaagaatctgaacgagagcctgatcgacctgcaagaactgggcaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggctcctgctgcaagttcgacgaggacgattctgagcccgtgctgaagggcgtgaaactgcactacacctga

SEQ ID NO: 5 - protein, 32 aa, Unknown
  tPA amino acid sequence (P->A mutant)
MDAMKRGLCCVLLLCGAVFVSASQEIHARFRR

SEQ ID NO: 6 - protein, 32 aa, Unknown
  tPA amino acid sequence (naturally occurring P)
MDAMKRGLCCVLLLCGAVFVSPSQEIHARFRR

SEQ ID NO: 7 - protein, 30 aa, Unknown
  tPA amino acid sequence SEQ ID NO: 5 without 'RR'
MDAMKRGLCCVLLLCGAVFVSASQEIHARF

SEQ ID NO: 8 - protein, 30 aa, Unknown
  tPA amino acid sequence SEQ ID NO: 6 without 'RR'
MDAMKRGLCCVLLLCGAVFVSPSQEIHARF

SEQ ID NO: 9 - DNA, 96 bp, Unknown
  nucleotide sequence encoding tPA SEQ ID NO: 5, which has been codon optimised for human codon usage
atggacgccatgaagaggggcctgtgctgcgtgctgctgctgtgtggcgccgtgtttgtgtccgccagccaggaaatccacgcccggttcagacgg

SEQ ID NO: 10 - protein, 1304 aa, Unknown
  Amino acid sequence of tPA-Spike fusion (tPA underlined) (i.e. amino acid sequence encoded by SEQ ID NO: 4) (reference sequence) (AZD1222)
MDAMKRGLCCVLLLCGAVFVSASQEIHARFRRFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 11 - DNA, 3822 bp, Unknown
  Nucleotide sequence from SARS-CoV2 (nCoV 19) genome for spike protein (From Accession number MG772933.1)
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 12 - protein, 1301 aa, Unknown
  amino acid sequence of tPA - 3990 Spike Protein fusion (tPA underlined) (Proline substitutions relative to SEQ ID NO: 1/SEQ ID NO: 3 in bold) (AZD3990)
MDAMKRGLCCVLLLCGAVFVSASQEIHARFRRFVFLVLLPLVSSQCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 13 - DNA, 44095 bp, Unknown
  AZD2816 plasmid : p5841 pDEST ChAdOx1 nCoV-19E DNA Sequence
gcggccgccaggcctacccactagtcaattcgggaggatcgaaacggcagatcgcaaaaaacagtacatacagaaggagacatgaacatgaacatcaaaaaaattgtaaaacaagccacagttctgacttttacgactgcacttctggcaggaggagcgactcaagccttcgcgaaagaaaataaccaaaaagcatacaaagaaacgtacggcgtctctcatattacacgccatgatatgctgcagatccctaaacagcagcaaaacgaaaaataccaagtgcctcaattcgatcaatcaacgattaaaaatattgagtctgcaaaaggacttgatgtgtgggacagctggccgctgcaaaacgctgacggaacagtagctgaatacaacggctatcacgttgtgtttgctcttgcgggaagcccgaaagacgctgatgacacatcaatctacatgttttatcaaaaggtcggcgacaactcaatcgacagctggaaaaacgcgggccgtgtctttaaagacagcgataagttcgacgccaacgatccgatcctgaaagatcagacgcaagaatggtccggttctgcaacctttacatctgacggaaaaatccgtttattctacactgactattccggtaaacattacggcaaacaaagcctgacaacagcgcaggtaaatgtgtcaaaatctgatgacacactcaaaatcaacggagtggaagatcacaaaacgatttttgacggagacggaaaaacatatcagaacgttcagcagtttatcgatgaaggcaattatacatccggcgacaaccatacgctgagagaccctcactacgttgaagacaaaggccataaataccttgtattcgaagccaacacgggaacagaaaacggataccaaggcgaagaatctttatttaacaaagcgtactacggcggcggcacgaacttcttccgtaaagaaagccagaagcttcagcagagcgctaaaaaacgcgatgctgagttagcgaacggcgccctcggtatcatagagttaaataatgattacacattgaaaaaagtaatgaagccgctgatcacttcaaacacggtaactgatgaaatcgagcgcgcgaatgttttcaaaatgaacggcaaatggtacttgttcactgattcacgcggttcaaaaatgacgatcgatggtattaactcaaacgatatttacatgcttggttatgtatcaaactctttaaccggcccttacaagccgctgaacaaaacagggcttgtgctgcaaatgggtcttgatccaaacgatgtgacattcacttactctcacttcgcagtgccgcaagccaaaggcaacaatgtggttatcacaagctacatgacaaacagaggcttcttcgaggataaaaaggcaacatttgcgccaagcttcttaatgaacatcaaaggcaataaaacatccgttgtcaaaaacagcatcctggagcaaggacagctgacagtcaactaataacagcaaaaagaaaatgccgatacttcattggcattttcttttatttctcaacaagatggtgaattgactagtgggtagatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattgatccggaacccttaatataacttcgtataatgtatgctatacgaagttattaggtccctcgactatagggtcaccgtcgacagcgacacacttgcatcggatgcagcccggttaacgtgccggcacggcctgggtaaccaggtattttgtccacataaccgtgcgcaaaatgttgtggataagcaggacacagcagcaatccacagcaggcatacaaccgcacaccgaggttactccgttctacaggttacgacgacatgtcaatacttgcccttgacaggcattgatggaatcgtagtctcacgctgatagtctgatcgacaatacaagtgggaccgtggtcccagaccgataatcagaccgacaacacgagtgggatcgtggtcccagactaataatcagaccgacgatacgagtgggaccgtggtcccagactaataatcagaccgacgatacgagtgggaccgtggttccagactaataatcagaccgacgatacgagtgggaccgtggtcccagacataataatcagaccgaaccgtggtcccagtctgattatcagaccgacgatacgagtgggaccgtggtcccagactaataatcagaccgacgatacgagtgggaccgtggtcccagactaataatcagaccgacgatacgagtgggaccgtggtcccagtctgattatcagaccgacgatacaagtggaacagtgggcccagagagaatattcaggccagttatgctttctggcctgtaacaaaggacattaagtaaagacagataaacgtagactaaaacgtggtcgcatcagggtgctggcttttcaagttccttaagaatggcctcaattttctctatacactcagttggaacacgagacctgtccaggttaagcaccattttatcgcccttatacaatactgtcgctccaggagcaaactgatgtcgtgagcttaaactagttcttgatgcagatgacgttttaagcacagaagttaaaagagtgataacttcttcagcttcaaatatcaccccagcttttttctgctcatgaaggttagatgcctgctgcttaagtaattcctctttatctgtaaaggctttttgaagtgcatcacctgaccgggcagatagttcaccggggtgagaaaaaagagcaacaactgatttaggcaatttggcggtgttgatacagcgggtaataatcttacgtgaaatattttccgcatcagccagcgcagaaatatttccagcaaattcattctgcaatcggcttgcataacgctgaccacgttcataagcacttgttgggcgataatcgttacccaatctggataatgcagccatctgctcatcatccagctcgccaaccagaacacgataatcactttcggtaagtgcagcagctttacgacggcgactcccatcggcaatttctatgacaccagatactcttcgaccgaacgccggtgtctgttgaccagtcagtagaaaagaagggatgagatcatccagtgcgtcctcagtaagcagctcctggtcacgttcattacctgaccatacccgagaggtcttctcaacactatcaccccggagcacttcaagagtaaacttcacatcccgaccacatacaggcaaagtaatggcattaccgcgagccattactcctacgcgcgcaattaacgaatccaccatcggggcagctggtgtcgataacgaagtatcttcaaccggttgagtattgagcgtatgttttggaataacaggcgcacgcttcattatctaatctcccagcgtggtttaatcagacgatcgaaaatttcattgcagacaggttcccaaatagaaagagcatttctccaggcaccagttgaagagcgttgatcaatggcctgttcaaaaacagttctcatccggatctgacctttaccaacttcatccgtttcacgtacaacattttttagaaccatgcttccccaggcatcccgaatttgctcctccatccacggggactgagagccattactattgctgtatttggtaagcaaaatacgtacatcaggctcgaaccctttaagatcaacgttcttgagcagatcacgaagcatatcgaaaaactgcagtgcggaggtgtagtcaaacaactcagcaggcgtgggaacaatcagcacatcagcagcacatacgacattaatcgtgccgatacccaggttaggcgcgctgtcaataactatgacatcatagtcatgagcaacagtttcaatggccagtcggagcatcaggtgtggatcggtgggcagtttaccttcatcaaatttgcccattaactcagtttcaatacggtgcagagccagacaggaaggaataatgtcaagccccggccagcaagtgggctttattgcataagtgacatcgtccttttccccaagatagaaaggcaggagagtgtcttctgcatgaatatgaagatctggtacccatccgtgatacattgaggctgttccctgggggtcgttaccttccacgagcaaaacacgtagccccttcagagccagatcctgagcaagatgaacagaaactgaggttttgtaaacgccacctttatgggcagcaaccccgatcaccggtggaaatacgtcttcagcacgtcgcaatcgcgtaccaaacacatcacgcatatgattaatttgttcaattgtataaccaacacgttgctcaacccgtcctcgaatttccatatccgggtgcggtagtcgccctgctttctcggcatctctgatagcctgagaagaaaccccaactaaatccgctgcttcacctattctccagcgccgggttattttcctcgcttccgggctgtcatcattaaactgtgcaatggcgatagccttcgtcatttcatgaccagcgtttatgcactggttaagtgtttccatgagtttcattctgaacatcctttaatcattgctttgcgtttttttattaaatcttgcaatttactgcaaagcaacaacaaaatcgcaaagtcatcaaaaaaccgcaaagttgtttaaaataagagcaacactacaaaaggagataagaagagcacatacctcagtcacttattatcactagcgctcgccgcagccgtgtaaccgagcatagcgagcgaactggcgaggaagcaaagaagaactgttctgtcagatagctcttacgctcagcgcaagaagaaatatccaccgtgggaaaaactccaggtagaggtacacacgcggatagccaattcagagtaataaactgtgataatcaaccctcatcaatgatgacgaactaacccccgatatcaggtcacatgacgaagggaaagagaaggaaatcaactgtgacaaactgccctcaaatttggcttccttaaaaattacagttcaaaaagtatgagaaaatccatgcaggctgaaggaaacagcaaaactgtgacaaattaccctcagtaggtcagaacaaatgtgacgaaccaccctcaaatctgtgacagataaccctcagactatcctgtcgtcatggaagtgatatcgcggaaggaaaatacgatatgagtcgtctggcggcctttctttttctcaatgtatgagaggcgcattggagttctgctgttgatctcattaacacagacctgcaggaagcggcggcggaagtcaggcatacgctggtaactttgaggcagctggtaacgctctatgatccagtcgattttcagagagacgatgcctgagccatccggcttacgatactgacacagggattcgtataaacgcatggcatacggattggtgatttcttttgtttcactaagccgaaactgcgtaaaccggttctgtaacccgataaagaagggaatgagatatgggttgatatgtacactgtaaagccctctggatggactgtgcgcacgtttgataaaccaaggaaaagattcatagcctttttcatcgccggcatcctcttcagggcgataaaaaaccacttccttccccgcgaaactcttcaatgcctgccgtatatccttactggcttccgcagaggtcaatccgaatatttcagcatatttagcaacatggatctcgcagataccgtcatgttcctgtagggtgccatcagattttctgatctggtcaacgaacagatacagcatacgtttttgatcccgggagagactatatgccgcctcagtgaggtcgtttgactggacgattcgcgggctatttttacgtttcttgtgattgataaccgctgtttccgccatgacagatccatgtgaagtgtgacaagtttttagattgtcacactaaataaaaaagagtcaataagcagggataactttgtgaaaaaacagcttcttctgagggcaatttgtcacagggttaagggcaatttgtcacagacaggactgtcatttgagggtgatttgtcacactgaaagggcaatttgtcacaacaccttctctagaaccagcatggataaaggcctacaaggcgctctaaaaaagaagatctaaaaactataaaaaaaataattataaaaatatccccgtggataagtggataaccccaagggaagttttttcaggcatcgtgtgtaagcagaatatataagtgctgttccctggtgcttcctcgctcactcgagggcttcgccctgtcgctcaactgcggcgagcactactggctgtaaaaggacagaccacatcatggttctgtgttcattaggttgttctgtccattgctgacataatccgctccacttcaacgtaacaccgcacgaagatttctattgttcctgaaggcatattcaaatcgttttcgttaccgcttgcaggcatcatgacagaacactacttcctataaacgctacacaggctcctgagattaataatgcggatctctacgataatgggagattttcccgactgtttcgttcgcttctcagtggataacagccagcttctctgtttaacagacaaaaacagcatatccactcagttccacatttccatataaaggccaaggcatttattctcaggataattgtttcagcatcgcaaccgcatcagactccggcatcgcaaactgcacccggtgccgggcagccacatccagcgcaaaaaccttcgtgtagacttccgttgaactgatggacttatgtcccatcaggctttgcagaactttcagcggtataccggcatacagcatgtgcatcgcataggaatggcggaacgtatgtggtgtgaccggaacagagaacgtcacaccgtcagcagcagcggcggcaaccgcctccccaatccaggtcctgaccgttctgtccgtcacttcccagatccgcgctttctctgtccttcctgtgcgacggttacgccgctccatgagcttatcgcgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggagttccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgatgataagctgtcaaacatgagaattggtcgacggcgcgccaaagcttgcatgcctgcagccgcgtaacctggcaaaatcggttacggttgagtaataaatggatgccctgcgtaagcggggcacatttcattacctctttctccgcacccgacatagataataacttcgtatagtatacattatacgaagttatctagtagacttaatcgcgtttaaacccatcatcaataatatacctcaaactttttgtgcgcgttaatatgcaaatgaggcgtttgaatttgggaagggaggaaggtgattggccgagagaagggcgaccgttaggggcggggcgagtgacgttttgatgacgtgaccgcgaggaggagccagtttgcaagttctcgtgggaaaagtgacgtcaaacgaggtgtggtttgaacacggaaatactcaattttcccgcgctctctgacaggaaatgaggtgtttctaggcggatgcaagtgaaaacgggccattttcgcgcgaaaactgaatgaggaagtgaaaatctgagtaatttcgcgtttatgacagggaggagtatttgccgagggccgagtagactttgaccgattacgtgggggtttcgattaccgtgtttttcacctaaatttccgcgtacggtgtcaaagtccggtgtttttacgtaggtgtcagctgatcgccagggtatttaaacctgcgctctccagtcaagaggccactcttgagtgccagcgagaagagttttctcctccgcgcgcgagtcagatctacactttgaaaggcgatcgctagcgacatcgatcacaagtttgtacaaaaaagcaggctccaccatgggaacccgcgttttgagatttctgtcgccgactaaattcatgtcgcgcgatagtggtgtttatcgccgatagagatggcgatattggaaaaatcgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacgaagcttggtaccttaatggacgccatgaagcgaggcctgtgctgtgttctgcttctgtgtggcgccgtgtttgtgtccgccagccaagagatccacgccagatttcggagattcgtgttcctggtgctgctgccactggtgtctagccagtgtgtgaactttaccaccaggacacagctgcctccagcctacaccaacagcttcaccagaggcgtgtactacccagacaaggtgttcagatccagcgtgctgcactctacccaggacctgttcctgcctttcttcagcaacgtgacctggttccacgccatccacgtgtccggcaccaatggcaccaagagattcgccaacccagtgctgcccttcaacgacggagtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgacccattcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacacctatcaacctcgtgcggggcctgcctcagggcttctctgctctggaaccactggtggatctgcccatcggcatcaacatcacccggtttcagacactgcacagaagctacctgacacctggcgatagcagctctggatggacagctggcgccgctgcctactatgtgggatacctgcagcctcggaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttcccgaatatcaccaatctgtgcccattcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcacctaccaagctgaacgacctgtgcttcacaaacgtgtacgccgacagcttcgtgatccgtggagatgaagtgcggcagattgcacctggacagacaggcaatatcgccgactacaactacaagctgcccgacgacttcaccggctgtgtgattgcctggaacagcaacaacctggatagcaaagtcggcggcaactacaattacctgtaccggctgttccggaagtccaatctgaagcccttcgagcgggacatctccaccgagatctatcaggccggcagcacaccttgtaacggcgtgaaaggcttcaactgctacttcccactgcagtcctacggctttcagcccacatatggcgtgggctaccagccttacagagtggtggtgctgagcttcgagctgctgcatgctcctgccacagtgtgcggccctaagaaaagcaccaatctcgtgaagaacaaatgcgtgaacttcaacttcaacggcctgaccggcaccggcgtgctgacagagagcaacaagaagttcctgccattccagcagttcggccgggatatcgccgataccacagatgccgtcagagatcctcagacactggaaatcctggacatcacaccttgcagcttcggcggagtgtctgtgatcacgcctggcaccaacaccagcaatcaggtggcagtgctgtaccagggtgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccctatcggcgctggcatctgcgcctcttaccagacacagacaaacagccctagacgggccagatctgtggccagccagagcatcattgcctacacaatgtctctgggcgtggagaacagcgtggcctactccaacaactctatcgctatcccgaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacgcctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggcgctgccctgcagatcccatttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtctagcaacttcggcgccatcagctctgtgctgaacgatatcctgagcagactggacaaggtggaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcgcctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagcctcagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccctgacgtggacctgggcgatatcagcggaatcaatgccagcgtcgtgaacatccagaaagagatcgaccggctgaacgaggtggccaagaatctgaacgagagcctgatcgacctgcaagaactgggcaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggctcctgctgcaagttcgacgaggacgattctgagcccgtgctgaagggcgtgaaactgcactacacctgataagcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgaggggggatcgatccgtcgagatatctagacccagctttcttgtacaaagtggtgatcgattcgacagatcgcgatcgcagtgagtagtgttctggggcgggggaggacctgcatgagggccagaatgactgaaatctgtgcttttctgtgtgttgcagcatcatgagcggaagcggctcctttgagggaggggtattcagcccttatctgacggggcgtctcccctcctgggcgggagtgcgtcagaatgtgatgggatccacggtggacggccggcccgtgcagcccgcgaactcttcaaccctgacctatgcaaccctgagctcttcgtcggtggacgcagctgccgccgcagctgctgcatccgccgccagcgccgtgcgcggaatggccatgggcgccggctactacggcactctggtggccaactcgagttccaccaataatcccgccagcctgaacgaggagaagctgctgctgctgatggcccagcttgaggccttgacccagcgcctgggcgagctgacccagcaggtggctcagctgcaggagcagacgcgggccgcggttgccacggtgaaatccaaataaaaaatgaatcaataaataaacggagacggttgttgattttaacacagagtctgaatctttatttgatttttcgcgcgcggtaggccctggaccaccggtctcgatcattgagcacccggtggatcttttccaggacccggtagaggtgggcttggatgttgaggtacatgggcatgagcccgtcccgggggtggaggtagctccattgcagggcctcgtgctcgggggtggtgttgtaaatcacccagtcatagcaggggcgcagggcgtggtgttgcacaatatctttgaggaggagactgatggccacgggcagccctttggtgtaggtgtttacaaatctgttgagctgggagggatgcatgcggggggagatgaggtgcatcttggcctggatcttgagattggcgatgttaccgcccagatcccgcctggggttcatgttgtgcaggaccaccagcacggtgtatccggtgcacttggggaatttatcatgcaacttggaagggaaggcgtgaaagaatttggcgacgcccttgtgtccgcccaggttttccatgcactcatccatgatgatggcaatgggcccgtgggcggcggcctgggcaaagacgtttcgggggtcggacacatcatagttgtggtcctgggtgaggtcatcataggccattttaatgaatttggggcggagggtgccggactgggggacaaaggtaccctcgatcccgggggcgtagttcccctcacagatctgcatctcccaggctttgagctcagagggggggatcatgtccacctgcggggcgataaagaacacggtttccggggcgggggagatgagctgggccgaaagcaagttccggagcagctgggacttgccgcagccggtggggccgtaaatgaccccgatgaccggctgcaggtggtagttgagggagagacagctgccgtcctcccggaggaggggggccacctcgttcatcatctcgcgcacgtgcatgttctcgcgcaccagttccgccaggaggcgctctccccccagagataggagctcctggagcgaggcgaagtttttcagcggcttgagtccgtcggccatgggcattttggagagggtctgttgcaagagttccaagcggtcccagagctcggtgatgtgctctacggcatctcgatccagcagacctcctcgtttcgcgggttgggacgactgcgggagtagggcaccagacgatgggcgtccagcgcagccagggtccggtccttccagggccgcagcgtccgcgtcagggtggtctccgtcacggtgaaggggtgcgcgccgggctgggcgcttgcgagggtgcgcttcaggctcatccggctggtcgaaaaccgctcccgatcggcgccctgcgcgtcggccaggtagcaattgaccatgagttcgtagttgagcgcctcggccgcgtggcctttggcgcggagcttacctttggaagtctgcccgcaggcgggacagaggagggacttgagggcgtagagcttgggggcgaggaagacggaatcgggggcgtaggcgtccgcgccgcagtgggcgcagacggtctcgcactccacgagccaggtgaggtcgggctggtcggggtcaaaaaccagtttcccgccgttctttttgatgcgtttcttacctttggtctccatgagctcgtgtccccgctgggtgacaaagaggctgtccgtgtccccgtagaccgactttatgggccggtcctcgagcggtgtgccgcggtcctcctcgtagaggaaccccgcccactccgagacgaaagcccgggtccaggccagcacgaaggaggccacgtgggacgggtagcggtcgttgtccaccagcgggtccactttttccagggtatgcaaacacatgtccccctcgtccacatccaggaaggtgattggcttgtaagtgtaggccacgtgaccgggggtcccggccgggggggtataaaagggggcgggcccctgctcgtcctcactgtcttccggatcgctgtccaggagcgccagctgttggggtaggtattccctctcgaaggcgggcatgacctcggcactcaggttgtcagtttctagaaacgaggaggatttgatattgacggtgccagcggagatgcctttcaagagcccctcgtccatctggtcagaaaagacgatttttttgttgtcgagcttggtggcgaaggagccgtagagggcgttggaaaggagcttggcgatggagcgcatggtctggtttttttccttgtcggcgcgctccttggccgcgatgttgagctgcacgtactcgcgcgccacgcacttccattcggggaagacggtggtcatctcgtcgggcacgattctgacctgccaacctcgattatgcagggtgatgaggtccacactggtggccacctcgccgcgcaggggctcgttggtccagcagaggcggccgcccttgcgcgagcagaaggggggcagagggtccagcatgacctcgtcgggggggtcggcatcgatggtgaagatgccgggcaggagatcggggtcgaagtagctgatggaagtggccagatcgtccagggaagcttgccattcgcgcacggccagcgcgcgctcgtagggactgaggggcgtgccccagggcatggggtgggtgagcgcggaggcgtacatgccgcagatgtcgtagacgtagaggggctcctcgaggatgccgatgtaggtggggtagcagcgccccccgcggatgctggcgcgcacgtagtcatacagctcgtgcgagggcgcgaggagccccgggcccaggttggtgcgactgggcttttcggcgcggtagacgatctggcgaaagatggcatgcgagttggaggagatggtgggcctttggaagatgttgaagtgggcgtgggggaggccgaccgagtcgcggatgaagtgggcgtaggagtcttgcagtttggcgacgagctcggcggtgacgaggacgtccagagcgcagtagtcgagggtctcctggatgatgtcatacttgagctggcccttttgtttccacagctcgcggttgagaaggaactcttcgcggtccttccagtactcttcgagggggaacccgtcctgatctgcacggtaagagcctagcatgtagaactggttgacggccttgtaggcgcagcagcccttctccacggggagggcgtaggcctgggcggccttgcgcagggaggtgtgcgtgagggcgaaggtgtccctgaccatgaccttgaggaactggtgcttgaaatcgatatcgtcgcagcccccctgctcccagagctggaagtccgtgcgcttcttgtaggcggggttgggcaaagcgaaagtaacatcgttgaaaaggatcttgcccgcgcggggcataaagttgcgagtgatgcggaaaggctggggcacctcggcccggttgttgatgacctgggcggcgagcacgatctcgtcgaaaccgttgatgttgtggcccacgatgtagagttccacgaatcgcgggcggcccttgacgtggggcagcttcttgagctcctcgtaggtgagctcgtcggggtcgctgagaccgtgctgctcgagcgcccagtcggcgagatgggggttggcgcggaggaaggaagtccagagatccacggccagggcggtttgcagacggtcccggtactgacggaactgctgcccgacggccattttttcgggggtgacgcagtagaaggtgcgggggtccccgtgccagcggtcccatttgagctggagggcgagatcgagggcgagctcgacgaggcggtcgtcccctgagagtttcatgaccagcatgaaggggacgagctgcttgccgaaggaccccatccaggtgtaggtttccacatcgtaggtgaggaagagcctttcggtgcgaggatgcgagccgatggggaagaactggatctcctgccaccaattggaggaatggctgttgatgtgatggaagtagaaatgccgacggcgcgccgaacactcgtgcttgtgtttatacaagcggccacagtgctcgcaacgctgcacgggatgcacgtgctgcacgagctgtacctgagttcctttgacgaggaatttcagtgggaagtggagtcgtggcgcctgcatctcgtgctgtactacgtcgtggtggtcggcctggccctcttctgcctcgatggtggtcatgctgacgagcccgcgcgggaggcaggtccagacctcggcgcgagcgggtcggagagcgaggacgagggcgcgcaggccggagctgtccagggtcctgagacgctgcggagtcaggtcagtgggcagcggcggcgcgcggttgacttgcaggagtttttccagggcgcgcgggaggtccagatggtacttgatctccaccgcgccgttggtggcgacgtcgatggcttgcagggtcccgtgcccctggggtgtgaccaccgtcccccgtttcttcttgggcggctggggcgacgggggcggtgcctcttccatggttagaagcggcggcgaggacgcgcgccgggcggcagaggcggctcggggcccggaggcaggggcggcaggggcacgtcggcgccgcgcgcgggtaggttctggtactgcgcccggagaagactggcgtgagcgacgacgcgacggttgacgtcctggatctgacgcctctgggtgaaggccacgggacccgtgagtttgaacctgaaagagagttcgacagaatcaatctcggtatcgttgacggcggcctgccgcaggatctcttgcacgtcgcccgagttgtcctggtaggcgatctcggtcatgaactgctcgatctcctcctcctgaaggtctccgcgaccggcgcgctccacggtggccgcgaggtcgttggagatgcggcccatgagctgcgagaaggcgttcatgcccgcctcgttccagacgcggctgtagaccacgacgccctcgggatcgcgggcgcgcatgaccacctgggcgaggttgagctccacgtggcgcgtgaagaccgcgtagttgcagaggcgctggtagaggtagttgagcgtggtggcgatgtgctcggtgacgaagaaatacatgatccagcggcggagcggcatctcgctgacgtcgcccagcgcctccaagcgttccatggcctcgtaaaagtccacggcgaagttgaaaaactgggagttgcgcgccgagacggtcaactcctcctccagaagacggatgagctcggcgatggtggcgcgcacctcgcgctcgaaggcccccgggagttcctccacttcctcctcttcttcctcctccactaacatctcttctacttcctcctcaggcggtggtggtggcgggggagggggcctgcgtcgccggcggcgcacgggcagacggtcgatgaagcgctcgatggtctcgccgcgccggcgtcgcatggtctcggtgacggcgcgcccgtcctcgcggggccgcagcgtgaagacgccgccgcgcatctccaggtggccgggggggtccccgttgggcagggagagggcgctgacgatgcatcttatcaattgccccgtagggactccgcgcaaggacctgagcgtctcgagatccacgggatctgaaaaccgttgaacgaaggcttcgagccagtcgcagtcgcaaggtaggctgagcacggtttcttctgccgggtcatgttggggagcggggcgggcgatgctgctggtgatgaagttgaaataggcggttctgagacggcggatggtggcgaggagcaccaggtctttgggcccggcttgctggatgcgcagacggtcggccatgccccaggcgtggtcctgacacctggccaggtccttgtagtagtcctgcatgagccgctccacgggcacctcctcctcgcccgcgcggccgtgcatgcgcgtgagcccgaagccgcgctggggctggacgagcgccaggtcggcgacgacgcgctcggcgaggatggcctgctggatctgggtgagggtggtctggaagtcgtcaaagtcgacgaagcggtggtaggctccggtgttgatggtgtaggagcagttggccatgacggaccagttgacggtctggtggcccggacgcacgagctcgtggtacttgaggcgcgagtaggcgcgcgtgtcgaagatgtagtcgttgcaggtgcgcaccaggtactggtagccgatgaggaagtgcggcggcggctggcggtagagcggccatcgctcggtggcgggggcgccgggcgcgaggtcctcgagcatggtgcggtggtagccgtagatgtacctggacatccaggtgatgccggcggcggtggtggaggcgcgcgggaactcgcggacgcggttccagatgttgcgcagcggcaggaagtagttcatggtgggcacggtctggcccgtgaggcgcgcgcagtcgtggatgctctatacgggcaaaaacgaaagcggtcagcggctcgactccgtggcctggaggctaagcgaacgggttgggctgcgcgtgtaccccggttcgaatctcgaatcaggctggagccgcagctaacgtggtactggcactcccgtctcgacccaagcctgcaccaaccctccaggatacggaggcgggtcgttttgcaactttttttggaggccggaaatgaaactagtaagcgcggaaagcggccgaccgcgatggctcgctgccgtagtctggagaagaatcgccagggttgcgttgcggtgtgccccggttcgaggccggccggattccgcggctaacgagggcgtggctgccccgtcgtttccaagaccccatagccagccgacttctccagttacggagcgagcccctcttttgttttgtttgtttttgccagatgcatcccgtactgcggcagatgcgcccccaccaccctccaccgcaacaacagccccctcctccacagccggcgcttctgcccccgccccagcagcagcagcaacttccagccacgaccgccgcggccgccgtgagcggggctggacagacttctcagtatgatcacctggccttggaagagggcgaggggctggcgcgcctgggggcgtcgtcgccggagcggcacccgcgcgtgcagatgaaaagggacgctcgcgaggcctacgtgcccaagcagaacctgttcagagacaggagcggcgaggagcccgaggagatgcgcgcggcccggttccacgcggggcgggagctgcggcgcggcctggaccgaaagagggtgctgagggacgaggatttcgaggcggacgagctgacggggatcagccccgcgcgcgcgcacgtggccgcggccaacctggtcacggcgtacgagcagaccgtgaaggaggagagcaacttccaaaaatccttcaacaaccacgtgcgcaccctgatcgcgcgcgaggaggtgaccctgggcctgatgcacctgtgggacctgctggaggccatcgtgcagaaccccaccagcaagccgctgacggcgcagctgttcctggtggtgcagcatagtcgggacaacgaggcgttcagggaggcgctgctgaatatcaccgagcccgagggccgctggctcctggacctggtgaacattctgcagagcatcgtggtgcaggagcgcgggctgccgctgtccgagaagctggcggccatcaacttctcggtgctgagtctgggcaagtactacgctaggaagatctacaagaccccgtacgtgcccatagacaaggaggtgaagatcgacgggttttacatgcgcatgaccctgaaagtgctgaccctgagcgacgatctgggggtgtaccgcaacgacaggatgcaccgcgcggtgagcgccagcaggcggcgcgagctgagcgaccaggagctgatgcacagcctgcagcgggccctgaccggggccgggaccgagggggagagctactttgacatgggcgcggacctgcactggcagcccagccgccgggccttggaggcggcaggcggtcccccctacatagaagaggtggacgatgaggtggacgaggagggcgagtacctggaagactgatggcgcgaccgtatttttgctagatgcaacaacagccacctcctgatcccgcgatgcgggcggcgctgcagagccagccgtccggcattaactcctcggacgattggacccaggccatgcaacgcatcatggcgctgacgacccgcaaccccgaagcctttagacagcagccccaggccaaccggctctcggccatcctggaggccgtggtgccctcgcgctccaaccccacgcacgagaaggtcctggccatcgtgaacgcgctggtggagaacaaggccatccgcggcgacgaggccggcctggtgtacaacgcgctgctggagcgcgtggcccgctacaacagcaccaacgtgcagaccaacctggaccgcatggtgaccgacgtgcgcgaggccgtggcccagcgcgagcggttccaccgcgagtccaacctgggatccatggtggcgctgaacgccttcctcagcacccagcccgccaacgtgccccggggccaggaggactacaccaacttcatcagcgccctgcgcctgatggtgaccgaggtgccccagagcgaggtgtaccagtccgggccggactacttcttccagaccagtcgccagggcttgcagaccgtgaacctgagccaggcgttcaagaacttgcagggcctgtggggcgtgcaggccccggtcggggaccgcgcgacggtgtcgagcctgctgacgccgaactcgcgcctgctgctgctgctggtggcccccttcacggacagcggcagcatcaaccgcaactcgtacctgggctacctgattaacctgtaccgcgaggccatcggccaggcgcacgtggacgagcagacctaccaggagatcacccacgtgagccgcgccctgggccaggacgacccgggcaatctggaagccaccctgaactttttgctgaccaaccggtcgcagaagatcccgccccagtacacgctcagcgccgaggaggagcgcatcctgcgatacgtgcagcagagcgtgggcctgttcctgatgcaggagggggccacccccagcgccgcgctcgacatgaccgcgcgcaacatggagcccagcatgtacgccagcaaccgcccgttcatcaataaactgatggactacttgcatcgggcggccgccatgaactctgactatttcaccaacgccatcctgaatccccactggctcccgccgccggggttctacacgggcgagtacgacatgcccgaccccaatgacgggttcctgtgggacgatgtggacagcagcgtgttctccccccgaccgggtgctaacgagcgccccttgtggaagaaggaaggcagcgaccgacgcccgtcctcggcgctgtccggccgcgagggtgctgccgcggcggtgcccgaggccgccagtcctttcccgagcttgcccttctcgctgaacagtattcgcagcagcgagctgggcaggatcacgcgcccgcgcttgctgggcgaggaggagtacttgaatgactcgctgttgagacccgagcgggagaagaacttccccaataacgggatagagagcctggtggacaagatgagccgctggaagacgtatgcgcaggagcacagggacgatccgtcgcagggggccacgagccggggcagcgccgcccgtaaacgccggtggcacgacaggcagcggggactgatgtgggacgatgaggattccgccgacgacagcagcgtgttggacttgggtgggagtggtaacccgttcgctcacctgcgcccccgcatcgggcgcatgatgtaagagaaaccgaaaataaatgatactcaccaaggccatggcgaccagcgtgcgttcgtttcttctctgttgttgtatctagtatgatgaggcgtgcgtacccggagggtcctcctccctcgtacgagagcgtgatgcagcaggcgatggcggcggcggcggcgatgcagcccccgctggaggctccttacgtgcccccgcggtacctggcgcctacggaggggcggaacagcattcgttactcggagctggcacccttgtacgataccacccggttgtacctggtggacaacaagtcggcggacatcgcctcgctgaactaccagaacgaccacagcaacttcctgaccaccgtggtgcagaacaatgacttcacccccacggaggccagcacccagaccatcaactttgacgagcgctcgcggtggggcggtcagctgaaaaccatcatgcacaccaacatgcccaacgtgaacgagttcatgtacagcaacaagttcaaggcgcgggtgatggtctcccgcaagacccccaacggggtgacagtgacagatggtagtcaggatatcttggagtatgaatgggtggagtttgagctgcccgaaggcaacttctcggtgaccatgaccatcgacctgatgaacaacgccatcatcgacaattacttggcggtggggcggcagaacggggtcctggagagcgatatcggcgtgaagttcgacactaggaacttcaggctgggctgggaccccgtgaccgagctggtcatgcccggggtgtacaccaacgaggccttccaccccgatattgtcttgctgcccggctgcggggtggacttcaccgagagccgcctcagcaacctgctgggcattcgcaagaggcagcccttccaggagggcttccagatcatgtacgaggatctggaggggggcaacatccccgcgctcctggatgtcgacgcctatgagaaaagcaaggaggagagcgccgccgcggcgactgcagctgtagccaccgcctctaccgaggtcaggggcgataattttgccagccctgcagcagtggcagcggccgaggcggctgaaaccgaaagtaagatagtcattcagccggtggagaaggatagcaaggacaggagctacaacgtgctgccggacaagataaacaccgcctaccgcagctggtacctggcctacaactatggcgaccccgagaagggcgtgcgctcctggacgctgctcaccacctcggacgtcacctgcggcgtggagcaagtctactggtcgctgcccgacatgatgcaagacccggtcaccttccgctccacgcgtcaagttagcaactacccggtggtgggcgccgagctcctgcccgtctactccaagagcttcttcaacgagcaggccgtctactcgcagcagctgcgcgccttcacctcgctcacgcacgtcttcaaccgcttccccgagaaccagatcctcgtccgcccgcccgcgcccaccattaccaccgtcagtgaaaacgttcctgctctcacagatcacgggaccctgccgctgcgcagcagtatccggggagtccagcgcgtgaccgttactgacgccagacgccgcacctgcccctacgtctacaaggccctgggcatagtcgcgccgcgcgtcctctcgagccgcaccttctaaaaaatgtccattctcatctcgcccagtaataacaccggttggggcctgcgcgcgcccagcaagatgtacggaggcgctcgccaacgctccacgcaacaccccgtgcgcgtgcgcgggcacttccgcgctccctggggcgccctcaagggccgcgtgcggtcgcgcaccaccgtcgacgacgtgatcgaccaggtggtggccgacgcgcgcaactacacccccgccgccgcgcccgtctccaccgtggacgccgtcatcgacagcgtggtggccgacgcgcgccggtacgcccgcgccaagagccggcggcggcgcatcgcccggcggcaccggagcacccccgccatgcgcgcggcgcgagccttgctgcgcagggccaggcgcacgggacgcagggccatgctcagggcggccagacgcgcggcttcaggcgccagcgccggcaggacccggagacgcgcggccacggcggcggcagcggccatcgccagcatgtcccgcccgcggcgagggaacgtgtactgggtgcgcgacgccgccaccggtgtgcgcgtgcccgtgcgcacccgcccccctcgcacttgaagatgttcacttcgcgatgttgatgtgtcccagcggcgaggaggatgtccaagcgcaaattcaaggaagagatgctccaggtcatcgcgcctgagatctacggccccgcggtggtgaaggaggaaagaaagccccgcaaaatcaagcgggtcaaaaaggacaaaaaggaagaagatgacgatctggtggagtttgtgcgcgagttcgccccccggcggcgcgtgcagtggcgcgggcggaaagtgcacccggtgctgagacccggcaccaccgtggtcttcacgcccggcgagcgctccggcagcgcttccaagcgctcctacgacgaggtgtacggggacgaggacatcctcgagcaggcggccgagcgcctgggcgagtttgcttacggcaagcgcagccgccccgccctgaaggaagaggcggtgtccatcccgctggaccacggcaaccccacgccgagcctcaagcccgtgaccctgcagcaggtgctgccgagcgcagcgccgcgccgggggttcaagcgcgagggcgaggatctgtaccccaccatgcagctgatggtgcccaagcgccagaagctggaagacgtgctggagaccatgaaggtggacccggacgtgcagcccgaggtcaaggtgcggcccatcaagcaggtggccccgggcctgggcgtgcagaccgtggacatcaagatccccacggagcccatggaaacgcagaccgagcccatgatcaagcccagcaccagcaccatggaggtgcagacggatccctggatgccatcggctcctagccgaagaccccggcgcaagtacggcgcggccagcctgctgatgcccaactacgcgctgcatccttccatcatccccacgccgggctaccgcggcacgcgcttctaccgcggtcatacaaccagccgccgccgcaagaccaccacccgccgccgccgtcgccgcacagccgctgcatctacccctgccgccctggtgcggagagtgtaccgccgcggccgcgcgcctctgaccctaccgcgcgcgcgctaccacccgagcatcgccatttaaactttcgcctgctttgcagatggccctcacatgccgcctccgcgttcccattacgggctaccgaggaagaaaaccgcgccgtagaaggctggcggggaacgggatgcgtcgccaccaccatcggcggcggcgcgccatcagcaagcggttggggggaggcttcctgcccgcgctgatccccatcatcgccgcggcgatcggggcgatccccggcattgcttccgtggcggtgcaggcctctcagcgccactgagacacttggaaaacatcttgtaataaaccaatggactctgacgctcctggtcctgtgatgtgttttcgtagacagatggaagacatcaatttttcgtccctggctccgcgacacggcacgcggccgttcatgggcacctggagcgacatcggcaccagccaactgaacgggggcgccttcaattggagcagtctctggagcgggcttaagaatttcgggtccacgcttaaaacctatggcagcaaggcgtggaacagcaccacagggcaggcgctgagggataagctgaaagagcagaacttccagcagaaggtggtcgatgggctcgcctcgggcatcaacggggtggtggacctggccaaccaggccgtgcagcggcagatcaacagccgcctggacccggtgccgcccgccggctccgtggagatgccgcaggtggaggaggagctgcctcccctggacaagcggggcgagaagcgaccccgccccgacgcggaggagacgctgctgacgcacacggacgagccgcccccgtacgaggaggcggtgaaactgggtctgcccaccacgcggcccatcgcgcccctggccaccggggtgctgaaacccgaaagtaataagcccgcgaccctggacttgcctcctcccgcttcccgcccctctacagtggctaagcccctgccgccggtggccgtggcccgcgcgcgacccgggggctccgcccgccctcatgcgaactggcagagcactctgaacagcatcgtgggtctgggagtgcagagtgtgaagcgccgccgctgctattaaacctaccgtagcgcttaacttgcttgtctgtgtgtgtatgtattatgtcgccgctgtccgccagaaggaggagtgaagaggcgcgtcgccgagttgcaagatggccaccccatcgatgctgccccagtgggcgtacatgcacatcgccggacaggacgcttcggagtacctgagtccgggtctggtgcagttcgcccgcgccacagacacctacttcagtctggggaacaagtttaggaaccccacggtggcgcccacgcacgatgtgaccaccgaccgcagccagcggctgacgctgcgcttcgtgcccgtggaccgcgaggacaacacctactcgtacaaagtgcgctacacgctggccgtgggcgacaaccgcgtgctggacatggccagcacctactttgacatccgcggcgtgctggatcggggccctagcttcaaaccctactccggcaccgcctacaacagcctggctcccaagggagcgcccaattccagccagtgggagcaaaaaaaggcaggcaatggtgacactatggaaacacacacatttggtgtggccccaatgggcggtgagaatattacaatcgacggattacaaattggaactgacgctacagctgatcaggataaaccaatttatgctgacaaaacattccagcctgaacctcaagtaggagaagaaaattggcaagaaactgaaagcttttatggcggtagggctcttaaaaaagacacaagcatgaaaccttgctatggctcctatgctagacccaccaatgtaaagggaggtcaagctaaacttaaagttggagctgatggagttcctaccaaagaatttgacatagacctggctttctttgatactcccggtggcacagtgaatggacaagatgagtataaagcagacattgtcatgtataccgaaaacacgtatctggaaactccagacacgcatgtggtatacaaaccaggcaaggatgatgcaagttctgaaattaacctggttcagcagtccatgcccaatagacccaactatattgggttcagagacaactttattgggctcatgtattacaacagtactggcaatatgggggtgctggctggtcaggcctcacagctgaatgctgtggtcgacttgcaagacagaaacaccgagctgtcataccagctcttgcttgactctttgggtgacagaacccggtatttcagtatgtggaatcaggcggtggacagttatgatcctgatgtgcgcattattgaaaaccatggtgtggaagacgaacttcccaactattgcttccccctggatgggtctggcactaatgccgcttaccaaggtgtgaaagtaaaaaatggtaacgatggtgatgttgagagcgaatgggaaaatgatgatactgtcgcagctcgaaatcaattatgcaagggcaacatttttgccatggaaattaacctccaagccaacctgtggagaagtttcctctactcgaacgtggccctgtacctgcccgactcttacaagtacacgccagccaacatcaccctgcccaccaacaccaacacttatgattacatgaacgggagagtggtgcctccctcgctggtggacgcctacatcaacatcggggcgcgctggtcgctggaccccatggacaacgtcaatcccttcaaccaccaccgcaacgcgggcctgcgctaccgctccatgctcctgggcaacgggcgctacgtgcccttccacatccaggtgccccagaaatttttcgccatcaagagcctcctgctcctgcccgggtcctacacctacgagtggaacttccgcaaggacgtcaacatgatcctgcagagctccctcggcaacgacctgcgcacggacggggcctccatctccttcaccagcatcaacctctacgccaccttcttccccatggcgcacaacacggcctccacgctcgaggccatgctgcgcaacgacaccaacgaccagtccttcaacgactacctctcggcggccaacatgctctaccccatcccggccaacgccaccaacgtgcccatctccatcccctcgcgcaactgggccgccttccgcggctggtccttcacgcgcctcaagaccaaggagacgccctcgctgggctccgggttcgacccctacttcgtctactcgggctccatcccctacctcgacggcaccttctacctcaaccacaccttcaagaaggtctccatcaccttcgactcctccgtcagctggcccggcaacgaccggctcctgacgcccaacgagttcgaaatcaagcgcaccgtcgacggcgagggatacaacgtggcccagtgcaacatgaccaaggactggttcctggtccagatgctggcccactacaacatcggctaccagggcttctacgtgcccgagggctacaaggaccgcatgtactccttcttccgcaacttccagcccatgagccgccaggtggtggacgaggtcaactacaaggactaccaggccgtcaccctggcctaccagcacaacaactcgggcttcgtcggctacctcgcgcccaccatgcgccagggccagccctaccccgccaactacccgtacccgctcatcggcaagagcgccgtcaccagcgtcacccagaaaaagttcctctgcgacagggtcatgtggcgcatccccttctccagcaacttcatgtccatgggcgcgctcaccgacctcggccagaacatgctctatgccaactccgcccacgcgctagacatgaatttcgaagtcgaccccatggatgagtccacccttctctatgttgtcttcgaagtcttcgacgtcgtccgagtgcaccagccccaccgcggcgtcatcgaggccgtctacctgcgcacccccttctcggccggtaacgccaccacctaaattgctacttgcatgatggctgagcccacaggctccggcgagcaggagctcagggccatcatccgcgacctgggctgcgggccctacttcctgggcaccttcgataagcgcttcccgggattcatggccccgcacaagctggcctgcgccatcgtcaacacggccggccgcgagaccgggggcgagcactggctggccttcgcctggaacccgcgctcgaacacctgctacctcttcgaccccttcgggttctcggacgagcgcctcaagcagatctaccagttcgagtacgagggcctgctgcgccgtagcgccctggccaccgaggaccgctgcgtcaccctggaaaagtccacccagaccgtgcagggtccgcgctcggccgcctgcgggctcttctgctgcatgttcctgcacgccttcgtgcactggcccgaccgccccatggacaagaaccccaccatgaacttgctgacgggggtgcccaacggcatgctccagtcgccccaggtggaacccaccctgcgccgcaaccaggaggcgctctaccgcttcctcaactcccactccgcctactttcgctcccaccgcgcgcgcatcgagaaggccaccgccttcgaccgcatgaacaatcaagacatgtaaaccgtgtgtgtatgtttaaaatatcttttaataaacagcactttaatgttacacatgcatctgagatgattttattttagaaatcgaaagggttctgccgggtctcggcatggcccgcgggcagggacacgttgcggaactggtacttggccagccacttgaactcggggatcagcagtttgggcagcggggtgtcggggaaggagtcggtccacagcttccgcgtcagctgcagggcgcccagcaggtcgggcgcggagatcttgaaatcgcagttgggacccgcgttctgcgcgcgagagttgcggtacacggggttgcagcactggaacaccatcagggccgggtgcttcacgctcgccagcaccgccgcgtcggtgatgctctccacgtcgaggtcctcggcgttggccatcccgaagggggtcatcttgcaggtctgccttcccatggtgggcacgcacccgggcttgtggttgcaatcgcagtgcagggggatcagcatcatctgggcctggtcggcgttcatccccgggtacatggccttcatgaaagcctccaattgcctgaacgcctgctgggccttggctccctcggtgaagaagaccccgcaggacttgctagagaactggttggtggcacagccggcatcgtgcacgcagcagcgcgcgtcgttgttggccagctgcaccacgctgcgcccccagcggttctgggtgatcttggcccggtcggggttctccttcagcgcgcgctgcccgttctcgctcgccacatccatctcgatcatgtgctccttctggatcatggtggtcccgtgcaggcaccgcagtttgccctcggcctcggtgcacccgtgcagccacagcgcgcacccggtgcactcccagttcttgtgggcgatctgggaatgcgcgtgcacgaacccttgcaggaagcggcccatcatggtcgtcagggtcttgttgctagtgaaggtcaacgggatgccgcggtgctcctcgttgatgtacaggtggcagatgcggcggtacacctcgccctgctcgggcatcagttggaagttggctttcaggtcggtctccacgcggtagcggtccatcagcatagtcatgatttccatgcccttctcccaggccgagacgatgggcaggctcatagggttcttcaccatcatcttagcactagcagccgcggccagggggtcgctctcatccagggtctcaaagctccgcttgccgtccttctcggtgatccgcaccggggggtagctgaagcccacggccgccagctcctcctcggcctgtctttcgtcctcgctgtcctggctgacgtcctgcatgaccacatgcttggtcttgcggggtttcttcttgggcggcagtggcggcggagatgcttgtggcgagggggagcgcgagttctcgctcaccactactatctcttcctcttcttggtccgaggccacgcggcggtaggtatgtctcttcgggggcagaggcggaggcgacgggctctcgccgccgcgacttggcggatggctggcagagccccttccgcgttcgggggtgcgctcccggcggcgctctgactgacttcctccgcggccggccattgtgttctcctagggaggaacaacaagcatggagactcagccatcgccaacctcgccatctgcccccaccgccggcgacgagaagcagcagcagcagaatgaaagcttaaccgccccgccgcccagccccgcctccgacgcagccgcggtcccagacatgcaagagatggaggaatccatcgagattgacctgggctatgtgacgcccgcggagcatgaggaggagctggcagtgcgctttcaatcgtcaagccaggaagataaagaacagccagagcaggaagcagagaacgagcagagtcaggctgggctcgagcatggcgactacctccacctgagcggggaggaggacgcgctcatcaagcatctggcccggcaggccaccatcgtcaaggacgcgctgctcgaccgcaccgaggtgcccctcagcgtggaggagctcagccgcgcctacgagctcaacctcttctcgccgcgcgtgccccccaagcgccagcccaacggcacctgcgagcccaacccccgcctcaacttctacccggtcttcgcggtgcccgaggccctggccacctaccacatctttttcaagaaccaaaagatccccgtctcctgccgcgccaaccgcacccgcgccgacgccctcttcaacctgggtcccggcgcccgcctacctgatatcgcctccttggaagaggttcccaagatcttcgagggtctgggcagcgacgagactcgggccgcgaacgctctgcaaggagaaggaggaggagagcatgagcaccacagcgccctggtcgagttggaaggcgacaacgcgcggctggcggtgctcaaacgcacggtcgagctgacccatttcgcctacccggctctgaacctgcccccgaaagtcatgagcgcggtcatggaccaggtgctcatcaagcgcgcgtcgcccatctccgaggacgagggcatgcaagactccgaggagggcaagcccgtggtcagcgacgagcagctggcccggtggctgggtcctaatgctacccctcaaagtttggaagagcggcgcaagctcatgatggccgtggtcctggtgaccgtggagctggagtgcctgcgccgcttcttcgccgacgcggagaccctgcgcaaggtcgaggagaacctgcactacctcttcaggcacgggttcgtgcgccaggcctgcaagatctccaacgtggagctgaccaacctggtctcctacatgggcatcttgcacgagaaccgcctggggcagaacgtgctgcacaccaccctgcgcggggaggcccgccgcgactacatccgcgactgcgtctacctctacctctgccacacctggcagacgggcatgggcgtgtggcagcagtgtctggaggagcagaacctgaaagagctctgcaagctcctgcaaaagaacctcaagggtctgtggaccgggttcgacgagcggaccaccgcctcggacctggccgacctcatcttccccgagcgcctcaggctgacgctgcgcaacggcctgcccgactttatgagccaaagcatgttgcaaaactttcgctctttcatcctcgaacgctccggaatcctgcccgccacctgctccgcgctgccctcggacttcgtgccgctgaccttccgcgagtgccccccgccgctgtggagccactgctacctgctgcgcctggccaactacctggcctaccactcggacgtgatcgaggacgtcagcggcgagggcctgctcgagtgccactgccgctgcaacctctgcacgccgcaccgctccctggcctgcaacccccagctgctgagcgagacccagatcatcggcaccttcgagttgcaagggcccagcgagggcgagggagccaaggggggtctgaaactcaccccggggctgtggacctcggcctacttgcgcaagttcgtgcccgaggattaccatcccttcgagatcaggttctacgaggaccaatcccagccgcccaaggccgagctgtcggcctgcgtcatcacccagggggcgatcctggcccaattgcaagccatccagaaatcccgccaagaattcttgctgaaaaagggccgcggggtctacctcgacccccagaccggtgaggagctcaaccccggcttcccccaggatgccccgaggaaacaagaagctgaaagtggagctgccgcccgtggaggatttggaggaagactgggagaacagcagtcaggcagaggagatggaggaagactgggacagcactcaggcagaggaggacagcctgcaagacagtctggaggaagacgaggaggaggcagaggaggaggtggaagaagcagccgccgccagaccgtcgtcctcggcgggggagaaagcaagcagcacggataccatctccgctccgggtcggggtcccgctcggccccacagtagatgggacgagaccgggcgattcccgaaccccaccacccagaccggtaagaaggagcggcagggatacaagtcctggcgggggcacaaaaacgccatcgtctcctgcttgcaggcctgcgggggcaacatctccttcacccggcgctacctgctcttccaccgcggggtgaacttcccccgcaacatcttgcattactaccgtcacctccacagcccctactacttccaagaagaggcagcagcagcagaaaaagaccagaaaaccagctagaaaatccacagcggcggcagcggcaggtggactgaggatcgcggcgaacgagccggcgcagacccgggagctgaggaaccggatctttcccaccctctatgccatcttccagcagagtcgggggcaggagcaggaactgaaagtcaagaaccgttctctgcgctcgctcacccgcagttgtctgtatcacaagagcgaagaccaacttcagcgcactctcgaggacgccgaggctctcttcaacaagtactgcgcgctcactcttaaagagtagcccgcgcccgcccagtcgcagaaaaaggcgggaattacgtcacctgtgcccttcgccctagccgcctccacccagcaccgccatgagcaaagagattcccacgccttacatgtggagctaccagccccagatgggcctggccgccggcgccgcccaggactactccacccgcatgaattggctcagcgccgggcccgcgatgatctcacgggtgaatgacatccgcgcccaccgaaaccagatactcctagaacagtcagcgctcaccgccacgccccgcaatcacctcaatccgcgtaattggcccgccgccctggtgtaccaggaaattccccagcccacgaccgtactacttccgcgagacgcccaggccgaagtccagctgactaactcaggtgtccagctggcgggcggcgccaccctgtgtcgtcaccgccccgctcagggtataaagcggctggtgatccggggcagaggcacacagctcaacgacgaggtggtgagctcttcgctgggtctgcgacctgacggagtcttccaactcgccggatcggggagatcttccttcacgcctcgtcaggcggtcctgactttggagagttcgtcctcgcagccccgctcgggcggcatcggcactctccagttcgtggaggagttcactccctcggtctacttcaaccccttctccggctcccccggccactacccggacgagttcatcccgaactttgacgccatcagcgagtcggtggacggctacgattgattaattaatcaactaaccccttacccctttaccctccagtaaaaataaagattaaaaatgattgaattgatcaataaagaatcacttacttgaaatctgaaaccaggtctctgtccatgttttctgtcagcagcacttcactcccctcttcccaactctggtactgcaggccccggcgggctgcaaacttcctccacactctgaaggggatgtcaaattcctcctgtccctcaatcttcatttttatcttctatcagatgtccaaaaagcgcgcgcgggtggatgatggcttcgaccccgtgtacccctacgatgcagacaacgcaccgactgtgcccttcatcaaccctcccttcgtctcttcagatggattccaagaaaagcccctgggggtgttgtccctgcgactggccgaccccgtcaccaccaagaatggggctgtcaccctcaagctgggggagggggtggacctcgacgactcgggaaaactcatctccaaaaatgccaccaaggccactgcccctctcagtatttccaacggcaccatttcccttaacatggctgcccctttttacaacaacaatggaacgttaagtctcaatgtttctacaccattagcagtatttcccacttttaacactttaggtatcagtcttggaaacggtcttcaaacttctaataagttgctgactgtacagttaactcatcctcttacattcagctcaaatagcatcacagtaaaaacagacaaaggactctatattaattctagtggaaacagagggcttgaggctaacataagcctaaaaagaggactgatttttgatggtaatgctattgcaacataccttggaagtggtttagactatggatcctatgatagcgatgggaaaacaagacccatcatcaccaaaattggagcaggtttgaattttgatgctaataatgccatggctgtgaagctaggcacaggtttaagttttgactctgccggtgccttaacagctggaaacaaagaggatgacaagctaacactttggactacacctgacccaagccctaattgtcaattactttcagacagagatgccaaatttaccctatgtcttacaaaatgcggtagtcaaatactaggcactgttgcagtagctgctgttactgtaggttcagcactaaatccaattaatgacacagtaaaaagcgccatagtattccttagatttgactctgacggtgtgctcatgtcaaactcatcaatggtaggtgattactggaactttagggaaggacagaccacccaaagtgtggcctatacaaatgctgtgggattcatgcccaatctaggtgcatatcctaaaacccaaagcaaaacaccaaaaaatagtatagtaagtcaggtatatttaaatggagaaactactatgccaatgacactgacaataactttcaatggcactgatgaaaaagacacaacacctgtgagcacttactccatgacttttacatggcagtggactggagactataaggacaagaatattacctttgctaccaactcctttactttctcctacatggcccaagaataaaccctgcatgccaaccccattgttcccaccactatggaaaactctgaagcagaaaaaaataaagttcaagtgttttattgattcaacagttttctcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgattaactttattccaaacggtctcggagcacttcaaaatgcaggtcccggaggtggcacctctcgcccccactgtgttggtggaaaataacagccaggtcaaaggtgacacggttctcgagatgttccacggtggcttccagcaaagcctccacgcgcacatccagaaacaagaggacagcgaaagcgggagcgttttctaattcctcaatcatcatattacactcctgcaccatccccagataattttcatttttccagccttgaatgattcgtattagttcctgaggtaaatccaagccagccatgataaaaagctcgcgcagagcgccctccaccggcattcttaagcacaccctcataattccaagagattctgctcctggttcacctgcagcagattaacaatgggaatatcaaaatctctgccgcgatccctaagctcctccctcaacaataactgtatgtaatctttcatatcatctccgaaatttttagccatagggccgccaggaataagagcagggcaagccacattacagataaagcgaagtcctccccagtgagcattgccaaatgtaagattgaaataagcatgctggctagaccctgtgatatcttccagataactggacagaaaatcaggcaagcaatttttaagaaaatcaacaaaagaaaagtcgtccaggtgcaggtttagagcctcaggaacaacgatggaataagtgcaaggagtgcgttccagcatggttagtgtttttttggtgatctgtagaacaaaaaataaacatgcaatattaaaccatgctagcctggcgaacaggtgggtaaatcactctttccagcaccaggcaggctacggggtctccggcgcgaccctcgtagaagctgtcgccatgattgaaaagcatcaccgagagaccttcccggtggccggcatggatgattcgagaagaagcatacactccgggaacattggcatccgtgagtgaaaaaaagcgacctataaagcctcggggcactacaatgctcaatctcaattccagcaaagccaccccatgcggatggagcacaaaattggcaggtgcgtaaaaaatgtaattactcccctcctgcacaggcagcaaagcccccgctccctccagaaacacatacaaagcctcagcgtccatagcttaccgagcacggcaggcgcaagagtcagagaaaaggctgagctctaacctgactgcccgctcctgtgctcaatatatagccctaacctacactgacgtaaaggccaaagtctaaaaatacccgccaaaatgacacacacgcccagcacacgcccagaaaccggtgacacactcaaaaaaatacgtgcgcttcctcaaacgcccaaaccggcgtcatttccgggttcccacgctacgtcaccgctcagcgactttcaaattccgtcgaccgttaaaaacgtcactcgccccgcccctaacggtcgcccttctctcggccaatcaccttcctcccttcccaaattcaaacgcctcatttgcatattaacgcgcacaaaaagtttgaggtatatatttgaatgatggtttaaac

SEQ ID NO: 14 - DNA, 30960 bp, Unknown
  ChAdOx1: Viral vector based on Chimpanzee adenovirus C68
ccatcatcaataatatacctcaaactttttgtgcgcgttaatatgcaaatgaggcgtttgaatttgggaagggaggaaggtgattggccgagagaagggcgaccgttaggggcggggcgagtgacgttttgatgacgtgaccgcgaggaggagccagtttgcaagttctcgtgggaaaagtgacgtcaaacgaggtgtggtttgaacacggaaatactcaattttcccgcgctctctgacaggaaatgaggtgtttctaggcggatgcaagtgaaaacgggccattttcgcgcgaaaactgaatgaggaagtgaaaatctgagtaatttcgcgtttatgacagggaggagtatttgccgagggccgagtagactttgaccgattacgtgggggtttcgattaccgtgtttttcacctaaatttccgcgtacggtgtcaaagtccggtgtttttacgtaggtgtcagctgatcgccagggtatttaaacctgcgctctccagtcaagaggccactcttgagtgccagcgagaagagttttctcctccgcgcgcgagtcagatctacactttgaaaggcgatcgctagcgacatcgatcacaagtttgtacaaaaaagctgaacgagaaacgtaaaatgatataaatatcaatatattaaattagattttgcataaaaaacagactacataatactgtaaaacacaacatatccagtcactatggcggccgccgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgtgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcacgcgtggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggtgatcgattcgacagatcgcgatcgcagtgagtagtgttctggggcgggggaggacctgcatgagggccagaatgactgaaatctgtgcttttctgtgtgttgcagcatcatgagcggaagcggctcctttgagggaggggtattcagcccttatctgacggggcgtctcccctcctgggcgggagtgcgtcagaatgtgatgggatccacggtggacggccggcccgtgcagcccgcgaactcttcaaccctgacctatgcaaccctgagctcttcgtcggtggacgcagctgccgccgcagctgctgcatccgccgccagcgccgtgcgcggaatggccatgggcgccggctactacggcactctggtggccaactcgagttccaccaataatcccgccagcctgaacgaggagaagctgctgctgctgatggcccagcttgaggccttgacccagcgcctgggcgagctgacccagcaggtggctcagctgcaggagcagacgcgggccgcggttgccacggtgaaatccaaataaaaaatgaatcaataaataaacggagacggttgttgattttaacacagagtctgaatctttatttgatttttcgcgcgcggtaggccctggaccaccggtctcgatcattgagcacccggtggatcttttccaggacccggtagaggtgggcttggatgttgaggtacatgggcatgagcccgtcccgggggtggaggtagctccattgcagggcctcgtgctcgggggtggtgttgtaaatcacccagtcatagcaggggcgcagggcgtggtgttgcacaatatctttgaggaggagactgatggccacgggcagccctttggtgtaggtgtttacaaatctgttgagctgggagggatgcatgcggggggagatgaggtgcatcttggcctggatcttgagattggcgatgttaccgcccagatcccgcctggggttcatgttgtgcaggaccaccagcacggtgtatccggtgcacttggggaatttatcatgcaacttggaagggaaggcgtgaaagaatttggcgacgcccttgtgtccgcccaggttttccatgcactcatccatgatgatggcaatgggcccgtgggcggcggcctgggcaaagacgtttcgggggtcggacacatcatagttgtggtcctgggtgaggtcatcataggccattttaatgaatttggggcggagggtgccggactgggggacaaaggtaccctcgatcccgggggcgtagttcccctcacagatctgcatctcccaggctttgagctcagagggggggatcatgtccacctgcggggcgataaagaacacggtttccggggcgggggagatgagctgggccgaaagcaagttccggagcagctgggacttgccgcagccggtggggccgtaaatgaccccgatgaccggctgcaggtggtagttgagggagagacagctgccgtcctcccggaggaggggggccacctcgttcatcatctcgcgcacgtgcatgttctcgcgcaccagttccgccaggaggcgctctccccccagagataggagctcctggagcgaggcgaagtttttcagcggcttgagtccgtcggccatgggcattttggagagggtctgttgcaagagttccaagcggtcccagagctcggtgatgtgctctacggcatctcgatccagcagacctcctcgtttcgcgggttgggacgactgcgggagtagggcaccagacgatgggcgtccagcgcagccagggtccggtccttccagggccgcagcgtccgcgtcagggtggtctccgtcacggtgaaggggtgcgcgccgggctgggcgcttgcgagggtgcgcttcaggctcatccggctggtcgaaaaccgctcccgatcggcgccctgcgcgtcggccaggtagcaattgaccatgagttcgtagttgagcgcctcggccgcgtggcctttggcgcggagcttacctttggaagtctgcccgcaggcgggacagaggagggacttgagggcgtagagcttgggggcgaggaagacggaatcgggggcgtaggcgtccgcgccgcagtgggcgcagacggtctcgcactccacgagccaggtgaggtcgggctggtcggggtcaaaaaccagtttcccgccgttctttttgatgcgtttcttacctttggtctccatgagctcgtgtccccgctgggtgacaaagaggctgtccgtgtccccgtagaccgactttatgggccggtcctcgagcggtgtgccgcggtcctcctcgtagaggaaccccgcccactccgagacgaaagcccgggtccaggccagcacgaaggaggccacgtgggacgggtagcggtcgttgtccaccagcgggtccactttttccagggtatgcaaacacatgtccccctcgtccacatccaggaaggtgattggcttgtaagtgtaggccacgtgaccgggggtcccggccgggggggtataaaagggggcgggcccctgctcgtcctcactgtcttccggatcgctgtccaggagcgccagctgttggggtaggtattccctctcgaaggcgggcatgacctcggcactcaggttgtcagtttctagaaacgaggaggatttgatattgacggtgccagcggagatgcctttcaagagcccctcgtccatctggtcagaaaagacgatttttttgttgtcgagcttggtggcgaaggagccgtagagggcgttggaaaggagcttggcgatggagcgcatggtctggtttttttccttgtcggcgcgctccttggccgcgatgttgagctgcacgtactcgcgcgccacgcacttccattcggggaagacggtggtcatctcgtcgggcacgattctgacctgccaacctcgattatgcagggtgatgaggtccacactggtggccacctcgccgcgcaggggctcgttggtccagcagaggcggccgcccttgcgcgagcagaaggggggcagagggtccagcatgacctcgtcgggggggtcggcatcgatggtgaagatgccgggcaggagatcggggtcgaagtagctgatggaagtggccagatcgtccagggaagcttgccattcgcgcacggccagcgcgcgctcgtagggactgaggggcgtgccccagggcatggggtgggtgagcgcggaggcgtacatgccgcagatgtcgtagacgtagaggggctcctcgaggatgccgatgtaggtggggtagcagcgccccccgcggatgctggcgcgcacgtagtcatacagctcgtgcgagggcgcgaggagccccgggcccaggttggtgcgactgggcttttcggcgcggtagacgatctggcgaaagatggcatgcgagttggaggagatggtgggcctttggaagatgttgaagtgggcgtgggggaggccgaccgagtcgcggatgaagtgggcgtaggagtcttgcagtttggcgacgagctcggcggtgacgaggacgtccagagcgcagtagtcgagggtctcctggatgatgtcatacttgagctggcccttttgtttccacagctcgcggttgagaaggaactcttcgcggtccttccagtactcttcgagggggaacccgtcctgatctgcacggtaagagcctagcatgtagaactggttgacggccttgtaggcgcagcagcccttctccacggggagggcgtaggcctgggcggccttgcgcagggaggtgtgcgtgagggcgaaggtgtccctgaccatgaccttgaggaactggtgcttgaaatcgatatcgtcgcagcccccctgctcccagagctggaagtccgtgcgcttcttgtaggcggggttgggcaaagcgaaagtaacatcgttgaaaaggatcttgcccgcgcggggcataaagttgcgagtgatgcggaaaggctggggcacctcggcccggttgttgatgacctgggcggcgagcacgatctcgtcgaaaccgttgatgttgtggcccacgatgtagagttccacgaatcgcgggcggcccttgacgtggggcagcttcttgagctcctcgtaggtgagctcgtcggggtcgctgagaccgtgctgctcgagcgcccagtcggcgagatgggggttggcgcggaggaaggaagtccagagatccacggccagggcggtttgcagacggtcccggtactgacggaactgctgcccgacggccattttttcgggggtgacgcagtagaaggtgcgggggtccccgtgccagcggtcccatttgagctggagggcgagatcgagggcgagctcgacgaggcggtcgtcccctgagagtttcatgaccagcatgaaggggacgagctgcttgccgaaggaccccatccaggtgtaggtttccacatcgtaggtgaggaagagcctttcggtgcgaggatgcgagccgatggggaagaactggatctcctgccaccaattggaggaatggctgttgatgtgatggaagtagaaatgccgacggcgcgccgaacactcgtgcttgtgtttatacaagcggccacagtgctcgcaacgctgcacgggatgcacgtgctgcacgagctgtacctgagttcctttgacgaggaatttcagtgggaagtggagtcgtggcgcctgcatctcgtgctgtactacgtcgtggtggtcggcctggccctcttctgcctcgatggtggtcatgctgacgagcccgcgcgggaggcaggtccagacctcggcgcgagcgggtcggagagcgaggacgagggcgcgcaggccggagctgtccagggtcctgagacgctgcggagtcaggtcagtgggcagcggcggcgcgcggttgacttgcaggagtttttccagggcgcgcgggaggtccagatggtacttgatctccaccgcgccgttggtggcgacgtcgatggcttgcagggtcccgtgcccctggggtgtgaccaccgtcccccgtttcttcttgggcggctggggcgacgggggcggtgcctcttccatggttagaagcggcggcgaggacgcgcgccgggcggcagaggcggctcggggcccggaggcaggggcggcaggggcacgtcggcgccgcgcgcgggtaggttctggtactgcgcccggagaagactggcgtgagcgacgacgcgacggttgacgtcctggatctgacgcctctgggtgaaggccacgggacccgtgagtttgaacctgaaagagagttcgacagaatcaatctcggtatcgttgacggcggcctgccgcaggatctcttgcacgtcgcccgagttgtcctggtaggcgatctcggtcatgaactgctcgatctcctcctcctgaaggtctccgcgaccggcgcgctccacggtggccgcgaggtcgttggagatgcggcccatgagctgcgagaaggcgttcatgcccgcctcgttccagacgcggctgtagaccacgacgccctcgggatcgcgggcgcgcatgaccacctgggcgaggttgagctccacgtggcgcgtgaagaccgcgtagttgcagaggcgctggtagaggtagttgagcgtggtggcgatgtgctcggtgacgaagaaatacatgatccagcggcggagcggcatctcgctgacgtcgcccagcgcctccaagcgttccatggcctcgtaaaagtccacggcgaagttgaaaaactgggagttgcgcgccgagacggtcaactcctcctccagaagacggatgagctcggcgatggtggcgcgcacctcgcgctcgaaggcccccgggagttcctccacttcctcctcttcttcctcctccactaacatctcttctacttcctcctcaggcggtggtggtggcgggggagggggcctgcgtcgccggcggcgcacgggcagacggtcgatgaagcgctcgatggtctcgccgcgccggcgtcgcatggtctcggtgacggcgcgcccgtcctcgcggggccgcagcgtgaagacgccgccgcgcatctccaggtggccgggggggtccccgttgggcagggagagggcgctgacgatgcatcttatcaattgccccgtagggactccgcgcaaggacctgagcgtctcgagatccacgggatctgaaaaccgttgaacgaaggcttcgagccagtcgcagtcgcaaggtaggctgagcacggtttcttctgccgggtcatgttggggagcggggcgggcgatgctgctggtgatgaagttgaaataggcggttctgagacggcggatggtggcgaggagcaccaggtctttgggcccggcttgctggatgcgcagacggtcggccatgccccaggcgtggtcctgacacctggccaggtccttgtagtagtcctgcatgagccgctccacgggcacctcctcctcgcccgcgcggccgtgcatgcgcgtgagcccgaagccgcgctggggctggacgagcgccaggtcggcgacgacgcgctcggcgaggatggcctgctggatctgggtgagggtggtctggaagtcgtcaaagtcgacgaagcggtggtaggctccggtgttgatggtgtaggagcagttggccatgacggaccagttgacggtctggtggcccggacgcacgagctcgtggtacttgaggcgcgagtaggcgcgcgtgtcgaagatgtagtcgttgcaggtgcgcaccaggtactggtagccgatgaggaagtgcggcggcggctggcggtagagcggccatcgctcggtggcgggggcgccgggcgcgaggtcctcgagcatggtgcggtggtagccgtagatgtacctggacatccaggtgatgccggcggcggtggtggaggcgcgcgggaactcgcggacgcggttccagatgttgcgcagcggcaggaagtagttcatggtgggcacggtctggcccgtgaggcgcgcgcagtcgtggatgctctatacgggcaaaaacgaaagcggtcagcggctcgactccgtggcctggaggctaagcgaacgggttgggctgcgcgtgtaccccggttcgaatctcgaatcaggctggagccgcagctaacgtggtactggcactcccgtctcgacccaagcctgcaccaaccctccaggatacggaggcgggtcgttttgcaactttttttggaggccggaaatgaaactagtaagcgcggaaagcggccgaccgcgatggctcgctgccgtagtctggagaagaatcgccagggttgcgttgcggtgtgccccggttcgaggccggccggattccgcggctaacgagggcgtggctgccccgtcgtttccaagaccccatagccagccgacttctccagttacggagcgagcccctcttttgttttgtttgtttttgccagatgcatcccgtactgcggcagatgcgcccccaccaccctccaccgcaacaacagccccctcctccacagccggcgcttctgcccccgccccagcagcagcagcaacttccagccacgaccgccgcggccgccgtgagcggggctggacagacttctcagtatgatcacctggccttggaagagggcgaggggctggcgcgcctgggggcgtcgtcgccggagcggcacccgcgcgtgcagatgaaaagggacgctcgcgaggcctacgtgcccaagcagaacctgttcagagacaggagcggcgaggagcccgaggagatgcgcgcggcccggttccacgcggggcgggagctgcggcgcggcctggaccgaaagagggtgctgagggacgaggatttcgaggcggacgagctgacggggatcagccccgcgcgcgcgcacgtggccgcggccaacctggtcacggcgtacgagcagaccgtgaaggaggagagcaacttccaaaaatccttcaacaaccacgtgcgcaccctgatcgcgcgcgaggaggtgaccctgggcctgatgcacctgtgggacctgctggaggccatcgtgcagaaccccaccagcaagccgctgacggcgcagctgttcctggtggtgcagcatagtcgggacaacgaggcgttcagggaggcgctgctgaatatcaccgagcccgagggccgctggctcctggacctggtgaacattctgcagagcatcgtggtgcaggagcgcgggctgccgctgtccgagaagctggcggccatcaacttctcggtgctgagtctgggcaagtactacgctaggaagatctacaagaccccgtacgtgcccatagacaaggaggtgaagatcgacgggttttacatgcgcatgaccctgaaagtgctgaccctgagcgacgatctgggggtgtaccgcaacgacaggatgcaccgcgcggtgagcgccagcaggcggcgcgagctgagcgaccaggagctgatgcacagcctgcagcgggccctgaccggggccgggaccgagggggagagctactttgacatgggcgcggacctgcactggcagcccagccgccgggccttggaggcggcaggcggtcccccctacatagaagaggtggacgatgaggtggacgaggagggcgagtacctggaagactgatggcgcgaccgtatttttgctagatgcaacaacagccacctcctgatcccgcgatgcgggcggcgctgcagagccagccgtccggcattaactcctcggacgattggacccaggccatgcaacgcatcatggcgctgacgacccgcaaccccgaagcctttagacagcagccccaggccaaccggctctcggccatcctggaggccgtggtgccctcgcgctccaaccccacgcacgagaaggtcctggccatcgtgaacgcgctggtggagaacaaggccatccgcggcgacgaggccggcctggtgtacaacgcgctgctggagcgcgtggcccgctacaacagcaccaacgtgcagaccaacctggaccgcatggtgaccgacgtgcgcgaggccgtggcccagcgcgagcggttccaccgcgagtccaacctgggatccatggtggcgctgaacgccttcctcagcacccagcccgccaacgtgccccggggccaggaggactacaccaacttcatcagcgccctgcgcctgatggtgaccgaggtgccccagagcgaggtgtaccagtccgggccggactacttcttccagaccagtcgccagggcttgcagaccgtgaacctgagccaggcgttcaagaacttgcagggcctgtggggcgtgcaggccccggtcggggaccgcgcgacggtgtcgagcctgctgacgccgaactcgcgcctgctgctgctgctggtggcccccttcacggacagcggcagcatcaaccgcaactcgtacctgggctacctgattaacctgtaccgcgaggccatcggccaggcgcacgtggacgagcagacctaccaggagatcacccacgtgagccgcgccctgggccaggacgacccgggcaatctggaagccaccctgaactttttgctgaccaaccggtcgcagaagatcccgccccagtacacgctcagcgccgaggaggagcgcatcctgcgatacgtgcagcagagcgtgggcctgttcctgatgcaggagggggccacccccagcgccgcgctcgacatgaccgcgcgcaacatggagcccagcatgtacgccagcaaccgcccgttcatcaataaactgatggactacttgcatcgggcggccgccatgaactctgactatttcaccaacgccatcctgaatccccactggctcccgccgccggggttctacacgggcgagtacgacatgcccgaccccaatgacgggttcctgtgggacgatgtggacagcagcgtgttctccccccgaccgggtgctaacgagcgccccttgtggaagaaggaaggcagcgaccgacgcccgtcctcggcgctgtccggccgcgagggtgctgccgcggcggtgcccgaggccgccagtcctttcccgagcttgcccttctcgctgaacagtattcgcagcagcgagctgggcaggatcacgcgcccgcgcttgctgggcgaggaggagtacttgaatgactcgctgttgagacccgagcgggagaagaacttccccaataacgggatagagagcctggtggacaagatgagccgctggaagacgtatgcgcaggagcacagggacgatccgtcgcagggggccacgagccggggcagcgccgcccgtaaacgccggtggcacgacaggcagcggggactgatgtgggacgatgaggattccgccgacgacagcagcgtgttggacttgggtgggagtggtaacccgttcgctcacctgcgcccccgcatcgggcgcatgatgtaagagaaaccgaaaataaatgatactcaccaaggccatggcgaccagcgtgcgttcgtttcttctctgttgttgtatctagtatgatgaggcgtgcgtacccggagggtcctcctccctcgtacgagagcgtgatgcagcaggcgatggcggcggcggcggcgatgcagcccccgctggaggctccttacgtgcccccgcggtacctggcgcctacggaggggcggaacagcattcgttactcggagctggcacccttgtacgataccacccggttgtacctggtggacaacaagtcggcggacatcgcctcgctgaactaccagaacgaccacagcaacttcctgaccaccgtggtgcagaacaatgacttcacccccacggaggccagcacccagaccatcaactttgacgagcgctcgcggtggggcggtcagctgaaaaccatcatgcacaccaacatgcccaacgtgaacgagttcatgtacagcaacaagttcaaggcgcgggtgatggtctcccgcaagacccccaacggggtgacagtgacagatggtagtcaggatatcttggagtatgaatgggtggagtttgagctgcccgaaggcaacttctcggtgaccatgaccatcgacctgatgaacaacgccatcatcgacaattacttggcggtggggcggcagaacggggtcctggagagcgatatcggcgtgaagttcgacactaggaacttcaggctgggctgggaccccgtgaccgagctggtcatgcccggggtgtacaccaacgaggccttccaccccgatattgtcttgctgcccggctgcggggtggacttcaccgagagccgcctcagcaacctgctgggcattcgcaagaggcagcccttccaggagggcttccagatcatgtacgaggatctggaggggggcaacatccccgcgctcctggatgtcgacgcctatgagaaaagcaaggaggagagcgccgccgcggcgactgcagctgtagccaccgcctctaccgaggtcaggggcgataattttgccagccctgcagcagtggcagcggccgaggcggctgaaaccgaaagtaagatagtcattcagccggtggagaaggatagcaaggacaggagctacaacgtgctgccggacaagataaacaccgcctaccgcagctggtacctggcctacaactatggcgaccccgagaagggcgtgcgctcctggacgctgctcaccacctcggacgtcacctgcggcgtggagcaagtctactggtcgctgcccgacatgatgcaagacccggtcaccttccgctccacgcgtcaagttagcaactacccggtggtgggcgccgagctcctgcccgtctactccaagagcttcttcaacgagcaggccgtctactcgcagcagctgcgcgccttcacctcgctcacgcacgtcttcaaccgcttccccgagaaccagatcctcgtccgcccgcccgcgcccaccattaccaccgtcagtgaaaacgttcctgctctcacagatcacgggaccctgccgctgcgcagcagtatccggggagtccagcgcgtgaccgttactgacgccagacgccgcacctgcccctacgtctacaaggccctgggcatagtcgcgccgcgcgtcctctcgagccgcaccttctaaaaaatgtccattctcatctcgcccagtaataacaccggttggggcctgcgcgcgcccagcaagatgtacggaggcgctcgccaacgctccacgcaacaccccgtgcgcgtgcgcgggcacttccgcgctccctggggcgccctcaagggccgcgtgcggtcgcgcaccaccgtcgacgacgtgatcgaccaggtggtggccgacgcgcgcaactacacccccgccgccgcgcccgtctccaccgtggacgccgtcatcgacagcgtggtggccgacgcgcgccggtacgcccgcgccaagagccggcggcggcgcatcgcccggcggcaccggagcacccccgccatgcgcgcggcgcgagccttgctgcgcagggccaggcgcacgggacgcagggccatgctcagggcggccagacgcgcggcttcaggcgccagcgccggcaggacccggagacgcgcggccacggcggcggcagcggccatcgccagcatgtcccgcccgcggcgagggaacgtgtactgggtgcgcgacgccgccaccggtgtgcgcgtgcccgtgcgcacccgcccccctcgcacttgaagatgttcacttcgcgatgttgatgtgtcccagcggcgaggaggatgtccaagcgcaaattcaaggaagagatgctccaggtcatcgcgcctgagatctacggccccgcggtggtgaaggaggaaagaaagccccgcaaaatcaagcgggtcaaaaaggacaaaaaggaagaagatgacgatctggtggagtttgtgcgcgagttcgccccccggcggcgcgtgcagtggcgcgggcggaaagtgcacccggtgctgagacccggcaccaccgtggtcttcacgcccggcgagcgctccggcagcgcttccaagcgctcctacgacgaggtgtacggggacgaggacatcctcgagcaggcggccgagcgcctgggcgagtttgcttacggcaagcgcagccgccccgccctgaaggaagaggcggtgtccatcccgctggaccacggcaaccccacgccgagcctcaagcccgtgaccctgcagcaggtgctgccgagcgcagcgccgcgccgggggttcaagcgcgagggcgaggatctgtaccccaccatgcagctgatggtgcccaagcgccagaagctggaagacgtgctggagaccatgaaggtggacccggacgtgcagcccgaggtcaaggtgcggcccatcaagcaggtggccccgggcctgggcgtgcagaccgtggacatcaagatccccacggagcccatggaaacgcagaccgagcccatgatcaagcccagcaccagcaccatggaggtgcagacggatccctggatgccatcggctcctagccgaagaccccggcgcaagtacggcgcggccagcctgctgatgcccaactacgcgctgcatccttccatcatccccacgccgggctaccgcggcacgcgcttctaccgcggtcatacaaccagccgccgccgcaagaccaccacccgccgccgccgtcgccgcacagccgctgcatctacccctgccgccctggtgcggagagtgtaccgccgcggccgcgcgcctctgaccctaccgcgcgcgcgctaccacccgagcatcgccatttaaactttcgcctgctttgcagatggccctcacatgccgcctccgcgttcccattacgggctaccgaggaagaaaaccgcgccgtagaaggctggcggggaacgggatgcgtcgccaccaccatcggcggcggcgcgccatcagcaagcggttggggggaggcttcctgcccgcgctgatccccatcatcgccgcggcgatcggggcgatccccggcattgcttccgtggcggtgcaggcctctcagcgccactgagacacttggaaaacatcttgtaataaaccaatggactctgacgctcctggtcctgtgatgtgttttcgtagacagatggaagacatcaatttttcgtccctggctccgcgacacggcacgcggccgttcatgggcacctggagcgacatcggcaccagccaactgaacgggggcgccttcaattggagcagtctctggagcgggcttaagaatttcgggtccacgcttaaaacctatggcagcaaggcgtggaacagcaccacagggcaggcgctgagggataagctgaaagagcagaacttccagcagaaggtggtcgatgggctcgcctcgggcatcaacggggtggtggacctggccaaccaggccgtgcagcggcagatcaacagccgcctggacccggtgccgcccgccggctccgtggagatgccgcaggtggaggaggagctgcctcccctggacaagcggggcgagaagcgaccccgccccgacgcggaggagacgctgctgacgcacacggacgagccgcccccgtacgaggaggcggtgaaactgggtctgcccaccacgcggcccatcgcgcccctggccaccggggtgctgaaacccgaaagtaataagcccgcgaccctggacttgcctcctcccgcttcccgcccctctacagtggctaagcccctgccgccggtggccgtggcccgcgcgcgacccgggggctccgcccgccctcatgcgaactggcagagcactctgaacagcatcgtgggtctgggagtgcagagtgtgaagcgccgccgctgctattaaacctaccgtagcgcttaacttgcttgtctgtgtgtgtatgtattatgtcgccgctgtccgccagaaggaggagtgaagaggcgcgtcgccgagttgcaagatggccaccccatcgatgctgccccagtgggcgtacatgcacatcgccggacaggacgcttcggagtacctgagtccgggtctggtgcagttcgcccgcgccacagacacctacttcagtctggggaacaagtttaggaaccccacggtggcgcccacgcacgatgtgaccaccgaccgcagccagcggctgacgctgcgcttcgtgcccgtggaccgcgaggacaacacctactcgtacaaagtgcgctacacgctggccgtgggcgacaaccgcgtgctggacatggccagcacctactttgacatccgcggcgtgctggatcggggccctagcttcaaaccctactccggcaccgcctacaacagcctggctcccaagggagcgcccaattccagccagtgggagcaaaaaaaggcaggcaatggtgacactatggaaacacacacatttggtgtggccccaatgggcggtgagaatattacaatcgacggattacaaattggaactgacgctacagctgatcaggataaaccaatttatgctgacaaaacattccagcctgaacctcaagtaggagaagaaaattggcaagaaactgaaagcttttatggcggtagggctcttaaaaaagacacaagcatgaaaccttgctatggctcctatgctagacccaccaatgtaaagggaggtcaagctaaacttaaagttggagctgatggagttcctaccaaagaatttgacatagacctggctttctttgatactcccggtggcacagtgaatggacaagatgagtataaagcagacattgtcatgtataccgaaaacacgtatctggaaactccagacacgcatgtggtatacaaaccaggcaaggatgatgcaagttctgaaattaacctggttcagcagtccatgcccaatagacccaactatattgggttcagagacaactttattgggctcatgtattacaacagtactggcaatatgggggtgctggctggtcaggcctcacagctgaatgctgtggtcgacttgcaagacagaaacaccgagctgtcataccagctcttgcttgactctttgggtgacagaacccggtatttcagtatgtggaatcaggcggtggacagttatgatcctgatgtgcgcattattgaaaaccatggtgtggaagacgaacttcccaactattgcttccccctggatgggtctggcactaatgccgcttaccaaggtgtgaaagtaaaaaatggtaacgatggtgatgttgagagcgaatgggaaaatgatgatactgtcgcagctcgaaatcaattatgcaagggcaacatttttgccatggaaattaacctccaagccaacctgtggagaagtttcctctactcgaacgtggccctgtacctgcccgactcttacaagtacacgccagccaacatcaccctgcccaccaacaccaacacttatgattacatgaacgggagagtggtgcctccctcgctggtggacgcctacatcaacatcggggcgcgctggtcgctggaccccatggacaacgtcaatcccttcaaccaccaccgcaacgcgggcctgcgctaccgctccatgctcctgggcaacgggcgctacgtgcccttccacatccaggtgccccagaaatttttcgccatcaagagcctcctgctcctgcccgggtcctacacctacgagtggaacttccgcaaggacgtcaacatgatcctgcagagctccctcggcaacgacctgcgcacggacggggcctccatctccttcaccagcatcaacctctacgccaccttcttccccatggcgcacaacacggcctccacgctcgaggccatgctgcgcaacgacaccaacgaccagtccttcaacgactacctctcggcggccaacatgctctaccccatcccggccaacgccaccaacgtgcccatctccatcccctcgcgcaactgggccgccttccgcggctggtccttcacgcgcctcaagaccaaggagacgccctcgctgggctccgggttcgacccctacttcgtctactcgggctccatcccctacctcgacggcaccttctacctcaaccacaccttcaagaaggtctccatcaccttcgactcctccgtcagctggcccggcaacgaccggctcctgacgcccaacgagttcgaaatcaagcgcaccgtcgacggcgagggatacaacgtggcccagtgcaacatgaccaaggactggttcctggtccagatgctggcccactacaacatcggctaccagggcttctacgtgcccgagggctacaaggaccgcatgtactccttcttccgcaacttccagcccatgagccgccaggtggtggacgaggtcaactacaaggactaccaggccgtcaccctggcctaccagcacaacaactcgggcttcgtcggctacctcgcgcccaccatgcgccagggccagccctaccccgccaactacccgtacccgctcatcggcaagagcgccgtcaccagcgtcacccagaaaaagttcctctgcgacagggtcatgtggcgcatccccttctccagcaacttcatgtccatgggcgcgctcaccgacctcggccagaacatgctctatgccaactccgcccacgcgctagacatgaatttcgaagtcgaccccatggatgagtccacccttctctatgttgtcttcgaagtcttcgacgtcgtccgagtgcaccagccccaccgcggcgtcatcgaggccgtctacctgcgcacccccttctcggccggtaacgccaccacctaaattgctacttgcatgatggctgagcccacaggctccggcgagcaggagctcagggccatcatccgcgacctgggctgcgggccctacttcctgggcaccttcgataagcgcttcccgggattcatggccccgcacaagctggcctgcgccatcgtcaacacggccggccgcgagaccgggggcgagcactggctggccttcgcctggaacccgcgctcgaacacctgctacctcttcgaccccttcgggttctcggacgagcgcctcaagcagatctaccagttcgagtacgagggcctgctgcgccgtagcgccctggccaccgaggaccgctgcgtcaccctggaaaagtccacccagaccgtgcagggtccgcgctcggccgcctgcgggctcttctgctgcatgttcctgcacgccttcgtgcactggcccgaccgccccatggacaagaaccccaccatgaacttgctgacgggggtgcccaacggcatgctccagtcgccccaggtggaacccaccctgcgccgcaaccaggaggcgctctaccgcttcctcaactcccactccgcctactttcgctcccaccgcgcgcgcatcgagaaggccaccgccttcgaccgcatgaacaatcaagacatgtaaaccgtgtgtgtatgtttaaaatatcttttaataaacagcactttaatgttacacatgcatctgagatgattttattttagaaatcgaaagggttctgccgggtctcggcatggcccgcgggcagggacacgttgcggaactggtacttggccagccacttgaactcggggatcagcagtttgggcagcggggtgtcggggaaggagtcggtccacagcttccgcgtcagctgcagggcgcccagcaggtcgggcgcggagatcttgaaatcgcagttgggacccgcgttctgcgcgcgagagttgcggtacacggggttgcagcactggaacaccatcagggccgggtgcttcacgctcgccagcaccgccgcgtcggtgatgctctccacgtcgaggtcctcggcgttggccatcccgaagggggtcatcttgcaggtctgccttcccatggtgggcacgcacccgggcttgtggttgcaatcgcagtgcagggggatcagcatcatctgggcctggtcggcgttcatccccgggtacatggccttcatgaaagcctccaattgcctgaacgcctgctgggccttggctccctcggtgaagaagaccccgcaggacttgctagagaactggttggtggcacagccggcatcgtgcacgcagcagcgcgcgtcgttgttggccagctgcaccacgctgcgcccccagcggttctgggtgatcttggcccggtcggggttctccttcagcgcgcgctgcccgttctcgctcgccacatccatctcgatcatgtgctccttctggatcatggtggtcccgtgcaggcaccgcagtttgccctcggcctcggtgcacccgtgcagccacagcgcgcacccggtgcactcccagttcttgtgggcgatctgggaatgcgcgtgcacgaacccttgcaggaagcggcccatcatggtcgtcagggtcttgttgctagtgaaggtcaacgggatgccgcggtgctcctcgttgatgtacaggtggcagatgcggcggtacacctcgccctgctcgggcatcagttggaagttggctttcaggtcggtctccacgcggtagcggtccatcagcatagtcatgatttccatgcccttctcccaggccgagacgatgggcaggctcatagggttcttcaccatcatcttagcactagcagccgcggccagggggtcgctctcatccagggtctcaaagctccgcttgccgtccttctcggtgatccgcaccggggggtagctgaagcccacggccgccagctcctcctcggcctgtctttcgtcctcgctgtcctggctgacgtcctgcatgaccacatgcttggtcttgcggggtttcttcttgggcggcagtggcggcggagatgcttgtggcgagggggagcgcgagttctcgctcaccactactatctcttcctcttcttggtccgaggccacgcggcggtaggtatgtctcttcgggggcagaggcggaggcgacgggctctcgccgccgcgacttggcggatggctggcagagccccttccgcgttcgggggtgcgctcccggcggcgctctgactgacttcctccgcggccggccattgtgttctcctagggaggaacaacaagcatggagactcagccatcgccaacctcgccatctgcccccaccgccggcgacgagaagcagcagcagcagaatgaaagcttaaccgccccgccgcccagccccgcctccgacgcagccgcggtcccagacatgcaagagatggaggaatccatcgagattgacctgggctatgtgacgcccgcggagcatgaggaggagctggcagtgcgctttcaatcgtcaagccaggaagataaagaacagccagagcaggaagcagagaacgagcagagtcaggctgggctcgagcatggcgactacctccacctgagcggggaggaggacgcgctcatcaagcatctggcccggcaggccaccatcgtcaaggacgcgctgctcgaccgcaccgaggtgcccctcagcgtggaggagctcagccgcgcctacgagctcaacctcttctcgccgcgcgtgccccccaagcgccagcccaacggcacctgcgagcccaacccccgcctcaacttctacccggtcttcgcggtgcccgaggccctggccacctaccacatctttttcaagaaccaaaagatccccgtctcctgccgcgccaaccgcacccgcgccgacgccctcttcaacctgggtcccggcgcccgcctacctgatatcgcctccttggaagaggttcccaagatcttcgagggtctgggcagcgacgagactcgggccgcgaacgctctgcaaggagaaggaggaggagagcatgagcaccacagcgccctggtcgagttggaaggcgacaacgcgcggctggcggtgctcaaacgcacggtcgagctgacccatttcgcctacccggctctgaacctgcccccgaaagtcatgagcgcggtcatggaccaggtgctcatcaagcgcgcgtcgcccatctccgaggacgagggcatgcaagactccgaggagggcaagcccgtggtcagcgacgagcagctggcccggtggctgggtcctaatgctacccctcaaagtttggaagagcggcgcaagctcatgatggccgtggtcctggtgaccgtggagctggagtgcctgcgccgcttcttcgccgacgcggagaccctgcgcaaggtcgaggagaacctgcactacctcttcaggcacgggttcgtgcgccaggcctgcaagatctccaacgtggagctgaccaacctggtctcctacatgggcatcttgcacgagaaccgcctggggcagaacgtgctgcacaccaccctgcgcggggaggcccgccgcgactacatccgcgactgcgtctacctctacctctgccacacctggcagacgggcatgggcgtgtggcagcagtgtctggaggagcagaacctgaaagagctctgcaagctcctgcaaaagaacctcaagggtctgtggaccgggttcgacgagcggaccaccgcctcggacctggccgacctcatcttccccgagcgcctcaggctgacgctgcgcaacggcctgcccgactttatgagccaaagcatgttgcaaaactttcgctctttcatcctcgaacgctccggaatcctgcccgccacctgctccgcgctgccctcggacttcgtgccgctgaccttccgcgagtgccccccgccgctgtggagccactgctacctgctgcgcctggccaactacctggcctaccactcggacgtgatcgaggacgtcagcggcgagggcctgctcgagtgccactgccgctgcaacctctgcacgccgcaccgctccctggcctgcaacccccagctgctgagcgagacccagatcatcggcaccttcgagttgcaagggcccagcgagggcgagggagccaaggggggtctgaaactcaccccggggctgtggacctcggcctacttgcgcaagttcgtgcccgaggattaccatcccttcgagatcaggttctacgaggaccaatcccagccgcccaaggccgagctgtcggcctgcgtcatcacccagggggcgatcctggcccaattgcaagccatccagaaatcccgccaagaattcttgctgaaaaagggccgcggggtctacctcgacccccagaccggtgaggagctcaaccccggcttcccccaggatgccccgaggaaacaagaagctgaaagtggagctgccgcccgtggaggatttggaggaagactgggagaacagcagtcaggcagaggagatggaggaagactgggacagcactcaggcagaggaggacagcctgcaagacagtctggaggaagacgaggaggaggcagaggaggaggtggaagaagcagccgccgccagaccgtcgtcctcggcgggggagaaagcaagcagcacggataccatctccgctccgggtcggggtcccgctcggccccacagtagatgggacgagaccgggcgattcccgaaccccaccacccagaccggtaagaaggagcggcagggatacaagtcctggcgggggcacaaaaacgccatcgtctcctgcttgcaggcctgcgggggcaacatctccttcacccggcgctacctgctcttccaccgcggggtgaacttcccccgcaacatcttgcattactaccgtcacctccacagcccctactacttccaagaagaggcagcagcagcagaaaaagaccagaaaaccagctagaaaatccacagcggcggcagcggcaggtggactgaggatcgcggcgaacgagccggcgcagacccgggagctgaggaaccggatctttcccaccctctatgccatcttccagcagagtcgggggcaggagcaggaactgaaagtcaagaaccgttctctgcgctcgctcacccgcagttgtctgtatcacaagagcgaagaccaacttcagcgcactctcgaggacgccgaggctctcttcaacaagtactgcgcgctcactcttaaagagtagcccgcgcccgcccagtcgcagaaaaaggcgggaattacgtcacctgtgcccttcgccctagccgcctccacccagcaccgccatgagcaaagagattcccacgccttacatgtggagctaccagccccagatgggcctggccgccggcgccgcccaggactactccacccgcatgaattggctcagcgccgggcccgcgatgatctcacgggtgaatgacatccgcgcccaccgaaaccagatactcctagaacagtcagcgctcaccgccacgccccgcaatcacctcaatccgcgtaattggcccgccgccctggtgtaccaggaaattccccagcccacgaccgtactacttccgcgagacgcccaggccgaagtccagctgactaactcaggtgtccagctggcgggcggcgccaccctgtgtcgtcaccgccccgctcagggtataaagcggctggtgatccggggcagaggcacacagctcaacgacgaggtggtgagctcttcgctgggtctgcgacctgacggagtcttccaactcgccggatcggggagatcttccttcacgcctcgtcaggcggtcctgactttggagagttcgtcctcgcagccccgctcgggcggcatcggcactctccagttcgtggaggagttcactccctcggtctacttcaaccccttctccggctcccccggccactacccggacgagttcatcccgaactttgacgccatcagcgagtcggtggacggctacgattgattaattaatcaactaaccccttacccctttaccctccagtaaaaataaagattaaaaatgattgaattgatcaataaagaatcacttacttgaaatctgaaaccaggtctctgtccatgttttctgtcagcagcacttcactcccctcttcccaactctggtactgcaggccccggcgggctgcaaacttcctccacactctgaaggggatgtcaaattcctcctgtccctcaatcttcatttttatcttctatcagatgtccaaaaagcgcgcgcgggtggatgatggcttcgaccccgtgtacccctacgatgcagacaacgcaccgactgtgcccttcatcaaccctcccttcgtctcttcagatggattccaagaaaagcccctgggggtgttgtccctgcgactggccgaccccgtcaccaccaagaatggggctgtcaccctcaagctgggggagggggtggacctcgacgactcgggaaaactcatctccaaaaatgccaccaaggccactgcccctctcagtatttccaacggcaccatttcccttaacatggctgcccctttttacaacaacaatggaacgttaagtctcaatgtttctacaccattagcagtatttcccacttttaacactttaggtatcagtcttggaaacggtcttcaaacttctaataagttgctgactgtacagttaactcatcctcttacattcagctcaaatagcatcacagtaaaaacagacaaaggactctatattaattctagtggaaacagagggcttgaggctaacataagcctaaaaagaggactgatttttgatggtaatgctattgcaacataccttggaagtggtttagactatggatcctatgatagcgatgggaaaacaagacccatcatcaccaaaattggagcaggtttgaattttgatgctaataatgccatggctgtgaagctaggcacaggtttaagttttgactctgccggtgccttaacagctggaaacaaagaggatgacaagctaacactttggactacacctgacccaagccctaattgtcaattactttcagacagagatgccaaatttaccctatgtcttacaaaatgcggtagtcaaatactaggcactgttgcagtagctgctgttactgtaggttcagcactaaatccaattaatgacacagtaaaaagcgccatagtattccttagatttgactctgacggtgtgctcatgtcaaactcatcaatggtaggtgattactggaactttagggaaggacagaccacccaaagtgtggcctatacaaatgctgtgggattcatgcccaatctaggtgcatatcctaaaacccaaagcaaaacaccaaaaaatagtatagtaagtcaggtatatttaaatggagaaactactatgccaatgacactgacaataactttcaatggcactgatgaaaaagacacaacacctgtgagcacttactccatgacttttacatggcagtggactggagactataaggacaagaatattacctttgctaccaactcctttactttctcctacatggcccaagaataaaccctgcatgccaaccccattgttcccaccactatggaaaactctgaagcagaaaaaaataaagttcaagtgttttattgattcaacagttttctcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgattaactttattccaaacggtctcggagcacttcaaaatgcaggtcccggaggtggcacctctcgcccccactgtgttggtggaaaataacagccaggtcaaaggtgacacggttctcgagatgttccacggtggcttccagcaaagcctccacgcgcacatccagaaacaagaggacagcgaaagcgggagcgttttctaattcctcaatcatcatattacactcctgcaccatccccagataattttcatttttccagccttgaatgattcgtattagttcctgaggtaaatccaagccagccatgataaaaagctcgcgcagagcgccctccaccggcattcttaagcacaccctcataattccaagagattctgctcctggttcacctgcagcagattaacaatgggaatatcaaaatctctgccgcgatccctaagctcctccctcaacaataactgtatgtaatctttcatatcatctccgaaatttttagccatagggccgccaggaataagagcagggcaagccacattacagataaagcgaagtcctccccagtgagcattgccaaatgtaagattgaaataagcatgctggctagaccctgtgatatcttccagataactggacagaaaatcaggcaagcaatttttaagaaaatcaacaaaagaaaagtcgtccaggtgcaggtttagagcctcaggaacaacgatggaataagtgcaaggagtgcgttccagcatggttagtgtttttttggtgatctgtagaacaaaaaataaacatgcaatattaaaccatgctagcctggcgaacaggtgggtaaatcactctttccagcaccaggcaggctacggggtctccggcgcgaccctcgtagaagctgtcgccatgattgaaaagcatcaccgagagaccttcccggtggccggcatggatgattcgagaagaagcatacactccgggaacattggcatccgtgagtgaaaaaaagcgacctataaagcctcggggcactacaatgctcaatctcaattccagcaaagccaccccatgcggatggagcacaaaattggcaggtgcgtaaaaaatgtaattactcccctcctgcacaggcagcaaagcccccgctccctccagaaacacatacaaagcctcagcgtccatagcttaccgagcacggcaggcgcaagagtcagagaaaaggctgagctctaacctgactgcccgctcctgtgctcaatatatagccctaacctacactgacgtaaaggccaaagtctaaaaatacccgccaaaatgacacacacgcccagcacacgcccagaaaccggtgacacactcaaaaaaatacgtgcgcttcctcaaacgcccaaaccggcgtcatttccgggttcccacgctacgtcaccgctcagcgactttcaaattccgtcgaccgttaaaaacgtcactcgccccgcccctaacggtcgcccttctctcggccaatcaccttcctcccttcccaaattcaaacgcctcatttgcatattaacgcgcacaaaaagtttgaggtatatatttgaatgatg

SEQ ID NO: 15 - DNA, 44104 bp, Unknown
  AZD1222 plasmid : p5713 pDEST-ChAdOx1-nCOV-19 DNA sequence
gcggccgccaggcctacccactagtcaattcgggaggatcgaaacggcagatcgcaaaaaacagtacatacagaaggagacatgaacatgaacatcaaaaaaattgtaaaacaagccacagttctgacttttacgactgcacttctggcaggaggagcgactcaagccttcgcgaaagaaaataaccaaaaagcatacaaagaaacgtacggcgtctctcatattacacgccatgatatgctgcagatccctaaacagcagcaaaacgaaaaataccaagtgcctcaattcgatcaatcaacgattaaaaatattgagtctgcaaaaggacttgatgtgtgggacagctggccgctgcaaaacgctgacggaacagtagctgaatacaacggctatcacgttgtgtttgctcttgcgggaagcccgaaagacgctgatgacacatcaatctacatgttttatcaaaaggtcggcgacaactcaatcgacagctggaaaaacgcgggccgtgtctttaaagacagcgataagttcgacgccaacgatccgatcctgaaagatcagacgcaagaatggtccggttctgcaacctttacatctgacggaaaaatccgtttattctacactgactattccggtaaacattacggcaaacaaagcctgacaacagcgcaggtaaatgtgtcaaaatctgatgacacactcaaaatcaacggagtggaagatcacaaaacgatttttgacggagacggaaaaacatatcagaacgttcagcagtttatcgatgaaggcaattatacatccggcgacaaccatacgctgagagaccctcactacgttgaagacaaaggccataaataccttgtattcgaagccaacacgggaacagaaaacggataccaaggcgaagaatctttatttaacaaagcgtactacggcggcggcacgaacttcttccgtaaagaaagccagaagcttcagcagagcgctaaaaaacgcgatgctgagttagcgaacggcgccctcggtatcatagagttaaataatgattacacattgaaaaaagtaatgaagccgctgatcacttcaaacacggtaactgatgaaatcgagcgcgcgaatgttttcaaaatgaacggcaaatggtacttgttcactgattcacgcggttcaaaaatgacgatcgatggtattaactcaaacgatatttacatgcttggttatgtatcaaactctttaaccggcccttacaagccgctgaacaaaacagggcttgtgctgcaaatgggtcttgatccaaacgatgtgacattcacttactctcacttcgcagtgccgcaagccaaaggcaacaatgtggttatcacaagctacatgacaaacagaggcttcttcgaggataaaaaggcaacatttgcgccaagcttcttaatgaacatcaaaggcaataaaacatccgttgtcaaaaacagcatcctggagcaaggacagctgacagtcaactaataacagcaaaaagaaaatgccgatacttcattggcattttcttttatttctcaacaagatggtgaattgactagtgggtagatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattgatccggaacccttaatataacttcgtataatgtatgctatacgaagttattaggtccctcgactatagggtcaccgtcgacagcgacacacttgcatcggatgcagcccggttaacgtgccggcacggcctgggtaaccaggtattttgtccacataaccgtgcgcaaaatgttgtggataagcaggacacagcagcaatccacagcaggcatacaaccgcacaccgaggttactccgttctacaggttacgacgacatgtcaatacttgcccttgacaggcattgatggaatcgtagtctcacgctgatagtctgatcgacaatacaagtgggaccgtggtcccagaccgataatcagaccgacaacacgagtgggatcgtggtcccagactaataatcagaccgacgatacgagtgggaccgtggtcccagactaataatcagaccgacgatacgagtgggaccgtggttccagactaataatcagaccgacgatacgagtgggaccgtggtcccagacataataatcagaccgaaccgtggtcccagtctgattatcagaccgacgatacgagtgggaccgtggtcccagactaataatcagaccgacgatacgagtgggaccgtggtcccagactaataatcagaccgacgatacgagtgggaccgtggtcccagtctgattatcagaccgacgatacaagtggaacagtgggcccagagagaatattcaggccagttatgctttctggcctgtaacaaaggacattaagtaaagacagataaacgtagactaaaacgtggtcgcatcagggtgctggcttttcaagttccttaagaatggcctcaattttctctatacactcagttggaacacgagacctgtccaggttaagcaccattttatcgcccttatacaatactgtcgctccaggagcaaactgatgtcgtgagcttaaactagttcttgatgcagatgacgttttaagcacagaagttaaaagagtgataacttcttcagcttcaaatatcaccccagcttttttctgctcatgaaggttagatgcctgctgcttaagtaattcctctttatctgtaaaggctttttgaagtgcatcacctgaccgggcagatagttcaccggggtgagaaaaaagagcaacaactgatttaggcaatttggcggtgttgatacagcgggtaataatcttacgtgaaatattttccgcatcagccagcgcagaaatatttccagcaaattcattctgcaatcggcttgcataacgctgaccacgttcataagcacttgttgggcgataatcgttacccaatctggataatgcagccatctgctcatcatccagctcgccaaccagaacacgataatcactttcggtaagtgcagcagctttacgacggcgactcccatcggcaatttctatgacaccagatactcttcgaccgaacgccggtgtctgttgaccagtcagtagaaaagaagggatgagatcatccagtgcgtcctcagtaagcagctcctggtcacgttcattacctgaccatacccgagaggtcttctcaacactatcaccccggagcacttcaagagtaaacttcacatcccgaccacatacaggcaaagtaatggcattaccgcgagccattactcctacgcgcgcaattaacgaatccaccatcggggcagctggtgtcgataacgaagtatcttcaaccggttgagtattgagcgtatgttttggaataacaggcgcacgcttcattatctaatctcccagcgtggtttaatcagacgatcgaaaatttcattgcagacaggttcccaaatagaaagagcatttctccaggcaccagttgaagagcgttgatcaatggcctgttcaaaaacagttctcatccggatctgacctttaccaacttcatccgtttcacgtacaacattttttagaaccatgcttccccaggcatcccgaatttgctcctccatccacggggactgagagccattactattgctgtatttggtaagcaaaatacgtacatcaggctcgaaccctttaagatcaacgttcttgagcagatcacgaagcatatcgaaaaactgcagtgcggaggtgtagtcaaacaactcagcaggcgtgggaacaatcagcacatcagcagcacatacgacattaatcgtgccgatacccaggttaggcgcgctgtcaataactatgacatcatagtcatgagcaacagtttcaatggccagtcggagcatcaggtgtggatcggtgggcagtttaccttcatcaaatttgcccattaactcagtttcaatacggtgcagagccagacaggaaggaataatgtcaagccccggccagcaagtgggctttattgcataagtgacatcgtccttttccccaagatagaaaggcaggagagtgtcttctgcatgaatatgaagatctggtacccatccgtgatacattgaggctgttccctgggggtcgttaccttccacgagcaaaacacgtagccccttcagagccagatcctgagcaagatgaacagaaactgaggttttgtaaacgccacctttatgggcagcaaccccgatcaccggtggaaatacgtcttcagcacgtcgcaatcgcgtaccaaacacatcacgcatatgattaatttgttcaattgtataaccaacacgttgctcaacccgtcctcgaatttccatatccgggtgcggtagtcgccctgctttctcggcatctctgatagcctgagaagaaaccccaactaaatccgctgcttcacctattctccagcgccgggttattttcctcgcttccgggctgtcatcattaaactgtgcaatggcgatagccttcgtcatttcatgaccagcgtttatgcactggttaagtgtttccatgagtttcattctgaacatcctttaatcattgctttgcgtttttttattaaatcttgcaatttactgcaaagcaacaacaaaatcgcaaagtcatcaaaaaaccgcaaagttgtttaaaataagagcaacactacaaaaggagataagaagagcacatacctcagtcacttattatcactagcgctcgccgcagccgtgtaaccgagcatagcgagcgaactggcgaggaagcaaagaagaactgttctgtcagatagctcttacgctcagcgcaagaagaaatatccaccgtgggaaaaactccaggtagaggtacacacgcggatagccaattcagagtaataaactgtgataatcaaccctcatcaatgatgacgaactaacccccgatatcaggtcacatgacgaagggaaagagaaggaaatcaactgtgacaaactgccctcaaatttggcttccttaaaaattacagttcaaaaagtatgagaaaatccatgcaggctgaaggaaacagcaaaactgtgacaaattaccctcagtaggtcagaacaaatgtgacgaaccaccctcaaatctgtgacagataaccctcagactatcctgtcgtcatggaagtgatatcgcggaaggaaaatacgatatgagtcgtctggcggcctttctttttctcaatgtatgagaggcgcattggagttctgctgttgatctcattaacacagacctgcaggaagcggcggcggaagtcaggcatacgctggtaactttgaggcagctggtaacgctctatgatccagtcgattttcagagagacgatgcctgagccatccggcttacgatactgacacagggattcgtataaacgcatggcatacggattggtgatttcttttgtttcactaagccgaaactgcgtaaaccggttctgtaacccgataaagaagggaatgagatatgggttgatatgtacactgtaaagccctctggatggactgtgcgcacgtttgataaaccaaggaaaagattcatagcctttttcatcgccggcatcctcttcagggcgataaaaaaccacttccttccccgcgaaactcttcaatgcctgccgtatatccttactggcttccgcagaggtcaatccgaatatttcagcatatttagcaacatggatctcgcagataccgtcatgttcctgtagggtgccatcagattttctgatctggtcaacgaacagatacagcatacgtttttgatcccgggagagactatatgccgcctcagtgaggtcgtttgactggacgattcgcgggctatttttacgtttcttgtgattgataaccgctgtttccgccatgacagatccatgtgaagtgtgacaagtttttagattgtcacactaaataaaaaagagtcaataagcagggataactttgtgaaaaaacagcttcttctgagggcaatttgtcacagggttaagggcaatttgtcacagacaggactgtcatttgagggtgatttgtcacactgaaagggcaatttgtcacaacaccttctctagaaccagcatggataaaggcctacaaggcgctctaaaaaagaagatctaaaaactataaaaaaaataattataaaaatatccccgtggataagtggataaccccaagggaagttttttcaggcatcgtgtgtaagcagaatatataagtgctgttccctggtgcttcctcgctcactcgagggcttcgccctgtcgctcaactgcggcgagcactactggctgtaaaaggacagaccacatcatggttctgtgttcattaggttgttctgtccattgctgacataatccgctccacttcaacgtaacaccgcacgaagatttctattgttcctgaaggcatattcaaatcgttttcgttaccgcttgcaggcatcatgacagaacactacttcctataaacgctacacaggctcctgagattaataatgcggatctctacgataatgggagattttcccgactgtttcgttcgcttctcagtggataacagccagcttctctgtttaacagacaaaaacagcatatccactcagttccacatttccatataaaggccaaggcatttattctcaggataattgtttcagcatcgcaaccgcatcagactccggcatcgcaaactgcacccggtgccgggcagccacatccagcgcaaaaaccttcgtgtagacttccgttgaactgatggacttatgtcccatcaggctttgcagaactttcagcggtataccggcatacagcatgtgcatcgcataggaatggcggaacgtatgtggtgtgaccggaacagagaacgtcacaccgtcagcagcagcggcggcaaccgcctccccaatccaggtcctgaccgttctgtccgtcacttcccagatccgcgctttctctgtccttcctgtgcgacggttacgccgctccatgagcttatcgcgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggagttccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgatgataagctgtcaaacatgagaattggtcgacggcgcgccaaagcttgcatgcctgcagccgcgtaacctggcaaaatcggttacggttgagtaataaatggatgccctgcgtaagcggggcacatttcattacctctttctccgcacccgacatagataataacttcgtatagtatacattatacgaagttatctagtagacttaatcgcgtttaaacccatcatcaataatatacctcaaactttttgtgcgcgttaatatgcaaatgaggcgtttgaatttgggaagggaggaaggtgattggccgagagaagggcgaccgttaggggcggggcgagtgacgttttgatgacgtgaccgcgaggaggagccagtttgcaagttctcgtgggaaaagtgacgtcaaacgaggtgtggtttgaacacggaaatactcaattttcccgcgctctctgacaggaaatgaggtgtttctaggcggatgcaagtgaaaacgggccattttcgcgcgaaaactgaatgaggaagtgaaaatctgagtaatttcgcgtttatgacagggaggagtatttgccgagggccgagtagactttgaccgattacgtgggggtttcgattaccgtgtttttcacctaaatttccgcgtacggtgtcaaagtccggtgtttttacgtaggtgtcagctgatcgccagggtatttaaacctgcgctctccagtcaagaggccactcttgagtgccagcgagaagagttttctcctccgcgcgcgagtcagatctacactttgaaaggcgatcgctagcgacatcgatcacaagtttgtacaaaaaagcaggctccaccatgggaacccgcgttttgagatttctgtcgccgactaaattcatgtcgcgcgatagtggtgtttatcgccgatagagatggcgatattggaaaaatcgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacgaagcttggtaccttaatggacgccatgaagcgaggcctgtgctgtgttctgcttctgtgtggcgccgtgtttgtgtccgccagccaagagatccacgccagatttcggagattcgtgttcctggtgctgctgccactggtgtctagccagtgtgtgaacctgaccaccaggacacagctgcctccagcctacaccaacagcttcaccagaggcgtgtactacccagacaaggtgttcagatccagcgtgctgcactctacccaggacctgttcctgcctttcttcagcaacgtgacctggttccacgccatccacgtgtccggcaccaatggcaccaagagattcgacaacccagtgctgcccttcaacgacggagtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgacccattcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacacctatcaacctcgtgcgggatctgcctcagggcttctctgctctggaaccactggtggatctgcccatcggcatcaacatcacccggtttcagacactgctggccctgcacagaagctacctgacacctggcgatagcagctctggatggacagctggcgccgctgcctactatgtgggatacctgcagcctcggaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttcccgaatatcaccaatctgtgcccattcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcacctaccaagctgaacgacctgtgcttcacaaacgtgtacgccgacagcttcgtgatccgtggagatgaagtgcggcagattgcacctggacagacaggcaagatcgccgactacaactacaagctgcccgacgacttcaccggctgtgtgattgcctggaacagcaacaacctggatagcaaagtcggcggcaactacaattacctgtaccggctgttccggaagtccaatctgaagcccttcgagcgggacatctccaccgagatctatcaggccggcagcacaccttgtaacggcgtggaaggcttcaactgctacttcccactgcagtcctacggctttcagcccacaaatggcgtgggctaccagccttacagagtggtggtgctgagcttcgagctgctgcatgctcctgccacagtgtgcggccctaagaaaagcaccaatctcgtgaagaacaaatgcgtgaacttcaacttcaacggcctgaccggcaccggcgtgctgacagagagcaacaagaagttcctgccattccagcagttcggccgggatatcgccgataccacagatgccgtcagagatcctcagacactggaaatcctggacatcacaccttgcagcttcggcggagtgtctgtgatcacgcctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccctatcggcgctggcatctgcgcctcttaccagacacagacaaacagccctagacgggccagatctgtggccagccagagcatcattgcctacacaatgtctctgggcgccgagaacagcgtggcctactccaacaactctatcgctatcccgaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacgcctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggcgctgccctgcagatcccatttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtctagcaacttcggcgccatcagctctgtgctgaacgatatcctgagcagactggacaaggtggaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcgcctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagcctcagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccctgacgtggacctgggcgatatcagcggaatcaatgccagcgtcgtgaacatccagaaagagatcgaccggctgaacgaggtggccaagaatctgaacgagagcctgatcgacctgcaagaactgggcaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggctcctgctgcaagttcgacgaggacgattctgagcccgtgctgaagggcgtgaaactgcactacacctgataagcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgaggggggatcgatccgtcgagatatctagacccagctttcttgtacaaagtggtgatcgattcgacagatcgcgatcgcagtgagtagtgttctggggcgggggaggacctgcatgagggccagaatgactgaaatctgtgcttttctgtgtgttgcagcatcatgagcggaagcggctcctttgagggaggggtattcagcccttatctgacggggcgtctcccctcctgggcgggagtgcgtcagaatgtgatgggatccacggtggacggccggcccgtgcagcccgcgaactcttcaaccctgacctatgcaaccctgagctcttcgtcggtggacgcagctgccgccgcagctgctgcatccgccgccagcgccgtgcgcggaatggccatgggcgccggctactacggcactctggtggccaactcgagttccaccaataatcccgccagcctgaacgaggagaagctgctgctgctgatggcccagcttgaggccttgacccagcgcctgggcgagctgacccagcaggtggctcagctgcaggagcagacgcgggccgcggttgccacggtgaaatccaaataaaaaatgaatcaataaataaacggagacggttgttgattttaacacagagtctgaatctttatttgatttttcgcgcgcggtaggccctggaccaccggtctcgatcattgagcacccggtggatcttttccaggacccggtagaggtgggcttggatgttgaggtacatgggcatgagcccgtcccgggggtggaggtagctccattgcagggcctcgtgctcgggggtggtgttgtaaatcacccagtcatagcaggggcgcagggcgtggtgttgcacaatatctttgaggaggagactgatggccacgggcagccctttggtgtaggtgtttacaaatctgttgagctgggagggatgcatgcggggggagatgaggtgcatcttggcctggatcttgagattggcgatgttaccgcccagatcccgcctggggttcatgttgtgcaggaccaccagcacggtgtatccggtgcacttggggaatttatcatgcaacttggaagggaaggcgtgaaagaatttggcgacgcccttgtgtccgcccaggttttccatgcactcatccatgatgatggcaatgggcccgtgggcggcggcctgggcaaagacgtttcgggggtcggacacatcatagttgtggtcctgggtgaggtcatcataggccattttaatgaatttggggcggagggtgccggactgggggacaaaggtaccctcgatcccgggggcgtagttcccctcacagatctgcatctcccaggctttgagctcagagggggggatcatgtccacctgcggggcgataaagaacacggtttccggggcgggggagatgagctgggccgaaagcaagttccggagcagctgggacttgccgcagccggtggggccgtaaatgaccccgatgaccggctgcaggtggtagttgagggagagacagctgccgtcctcccggaggaggggggccacctcgttcatcatctcgcgcacgtgcatgttctcgcgcaccagttccgccaggaggcgctctccccccagagataggagctcctggagcgaggcgaagtttttcagcggcttgagtccgtcggccatgggcattttggagagggtctgttgcaagagttccaagcggtcccagagctcggtgatgtgctctacggcatctcgatccagcagacctcctcgtttcgcgggttgggacgactgcgggagtagggcaccagacgatgggcgtccagcgcagccagggtccggtccttccagggccgcagcgtccgcgtcagggtggtctccgtcacggtgaaggggtgcgcgccgggctgggcgcttgcgagggtgcgcttcaggctcatccggctggtcgaaaaccgctcccgatcggcgccctgcgcgtcggccaggtagcaattgaccatgagttcgtagttgagcgcctcggccgcgtggcctttggcgcggagcttacctttggaagtctgcccgcaggcgggacagaggagggacttgagggcgtagagcttgggggcgaggaagacggaatcgggggcgtaggcgtccgcgccgcagtgggcgcagacggtctcgcactccacgagccaggtgaggtcgggctggtcggggtcaaaaaccagtttcccgccgttctttttgatgcgtttcttacctttggtctccatgagctcgtgtccccgctgggtgacaaagaggctgtccgtgtccccgtagaccgactttatgggccggtcctcgagcggtgtgccgcggtcctcctcgtagaggaaccccgcccactccgagacgaaagcccgggtccaggccagcacgaaggaggccacgtgggacgggtagcggtcgttgtccaccagcgggtccactttttccagggtatgcaaacacatgtccccctcgtccacatccaggaaggtgattggcttgtaagtgtaggccacgtgaccgggggtcccggccgggggggtataaaagggggcgggcccctgctcgtcctcactgtcttccggatcgctgtccaggagcgccagctgttggggtaggtattccctctcgaaggcgggcatgacctcggcactcaggttgtcagtttctagaaacgaggaggatttgatattgacggtgccagcggagatgcctttcaagagcccctcgtccatctggtcagaaaagacgatttttttgttgtcgagcttggtggcgaaggagccgtagagggcgttggaaaggagcttggcgatggagcgcatggtctggtttttttccttgtcggcgcgctccttggccgcgatgttgagctgcacgtactcgcgcgccacgcacttccattcggggaagacggtggtcatctcgtcgggcacgattctgacctgccaacctcgattatgcagggtgatgaggtccacactggtggccacctcgccgcgcaggggctcgttggtccagcagaggcggccgcccttgcgcgagcagaaggggggcagagggtccagcatgacctcgtcgggggggtcggcatcgatggtgaagatgccgggcaggagatcggggtcgaagtagctgatggaagtggccagatcgtccagggaagcttgccattcgcgcacggccagcgcgcgctcgtagggactgaggggcgtgccccagggcatggggtgggtgagcgcggaggcgtacatgccgcagatgtcgtagacgtagaggggctcctcgaggatgccgatgtaggtggggtagcagcgccccccgcggatgctggcgcgcacgtagtcatacagctcgtgcgagggcgcgaggagccccgggcccaggttggtgcgactgggcttttcggcgcggtagacgatctggcgaaagatggcatgcgagttggaggagatggtgggcctttggaagatgttgaagtgggcgtgggggaggccgaccgagtcgcggatgaagtgggcgtaggagtcttgcagtttggcgacgagctcggcggtgacgaggacgtccagagcgcagtagtcgagggtctcctggatgatgtcatacttgagctggcccttttgtttccacagctcgcggttgagaaggaactcttcgcggtccttccagtactcttcgagggggaacccgtcctgatctgcacggtaagagcctagcatgtagaactggttgacggccttgtaggcgcagcagcccttctccacggggagggcgtaggcctgggcggccttgcgcagggaggtgtgcgtgagggcgaaggtgtccctgaccatgaccttgaggaactggtgcttgaaatcgatatcgtcgcagcccccctgctcccagagctggaagtccgtgcgcttcttgtaggcggggttgggcaaagcgaaagtaacatcgttgaaaaggatcttgcccgcgcggggcataaagttgcgagtgatgcggaaaggctggggcacctcggcccggttgttgatgacctgggcggcgagcacgatctcgtcgaaaccgttgatgttgtggcccacgatgtagagttccacgaatcgcgggcggcccttgacgtggggcagcttcttgagctcctcgtaggtgagctcgtcggggtcgctgagaccgtgctgctcgagcgcccagtcggcgagatgggggttggcgcggaggaaggaagtccagagatccacggccagggcggtttgcagacggtcccggtactgacggaactgctgcccgacggccattttttcgggggtgacgcagtagaaggtgcgggggtccccgtgccagcggtcccatttgagctggagggcgagatcgagggcgagctcgacgaggcggtcgtcccctgagagtttcatgaccagcatgaaggggacgagctgcttgccgaaggaccccatccaggtgtaggtttccacatcgtaggtgaggaagagcctttcggtgcgaggatgcgagccgatggggaagaactggatctcctgccaccaattggaggaatggctgttgatgtgatggaagtagaaatgccgacggcgcgccgaacactcgtgcttgtgtttatacaagcggccacagtgctcgcaacgctgcacgggatgcacgtgctgcacgagctgtacctgagttcctttgacgaggaatttcagtgggaagtggagtcgtggcgcctgcatctcgtgctgtactacgtcgtggtggtcggcctggccctcttctgcctcgatggtggtcatgctgacgagcccgcgcgggaggcaggtccagacctcggcgcgagcgggtcggagagcgaggacgagggcgcgcaggccggagctgtccagggtcctgagacgctgcggagtcaggtcagtgggcagcggcggcgcgcggttgacttgcaggagtttttccagggcgcgcgggaggtccagatggtacttgatctccaccgcgccgttggtggcgacgtcgatggcttgcagggtcccgtgcccctggggtgtgaccaccgtcccccgtttcttcttgggcggctggggcgacgggggcggtgcctcttccatggttagaagcggcggcgaggacgcgcgccgggcggcagaggcggctcggggcccggaggcaggggcggcaggggcacgtcggcgccgcgcgcgggtaggttctggtactgcgcccggagaagactggcgtgagcgacgacgcgacggttgacgtcctggatctgacgcctctgggtgaaggccacgggacccgtgagtttgaacctgaaagagagttcgacagaatcaatctcggtatcgttgacggcggcctgccgcaggatctcttgcacgtcgcccgagttgtcctggtaggcgatctcggtcatgaactgctcgatctcctcctcctgaaggtctccgcgaccggcgcgctccacggtggccgcgaggtcgttggagatgcggcccatgagctgcgagaaggcgttcatgcccgcctcgttccagacgcggctgtagaccacgacgccctcgggatcgcgggcgcgcatgaccacctgggcgaggttgagctccacgtggcgcgtgaagaccgcgtagttgcagaggcgctggtagaggtagttgagcgtggtggcgatgtgctcggtgacgaagaaatacatgatccagcggcggagcggcatctcgctgacgtcgcccagcgcctccaagcgttccatggcctcgtaaaagtccacggcgaagttgaaaaactgggagttgcgcgccgagacggtcaactcctcctccagaagacggatgagctcggcgatggtggcgcgcacctcgcgctcgaaggcccccgggagttcctccacttcctcctcttcttcctcctccactaacatctcttctacttcctcctcaggcggtggtggtggcgggggagggggcctgcgtcgccggcggcgcacgggcagacggtcgatgaagcgctcgatggtctcgccgcgccggcgtcgcatggtctcggtgacggcgcgcccgtcctcgcggggccgcagcgtgaagacgccgccgcgcatctccaggtggccgggggggtccccgttgggcagggagagggcgctgacgatgcatcttatcaattgccccgtagggactccgcgcaaggacctgagcgtctcgagatccacgggatctgaaaaccgttgaacgaaggcttcgagccagtcgcagtcgcaaggtaggctgagcacggtttcttctgccgggtcatgttggggagcggggcgggcgatgctgctggtgatgaagttgaaataggcggttctgagacggcggatggtggcgaggagcaccaggtctttgggcccggcttgctggatgcgcagacggtcggccatgccccaggcgtggtcctgacacctggccaggtccttgtagtagtcctgcatgagccgctccacgggcacctcctcctcgcccgcgcggccgtgcatgcgcgtgagcccgaagccgcgctggggctggacgagcgccaggtcggcgacgacgcgctcggcgaggatggcctgctggatctgggtgagggtggtctggaagtcgtcaaagtcgacgaagcggtggtaggctccggtgttgatggtgtaggagcagttggccatgacggaccagttgacggtctggtggcccggacgcacgagctcgtggtacttgaggcgcgagtaggcgcgcgtgtcgaagatgtagtcgttgcaggtgcgcaccaggtactggtagccgatgaggaagtgcggcggcggctggcggtagagcggccatcgctcggtggcgggggcgccgggcgcgaggtcctcgagcatggtgcggtggtagccgtagatgtacctggacatccaggtgatgccggcggcggtggtggaggcgcgcgggaactcgcggacgcggttccagatgttgcgcagcggcaggaagtagttcatggtgggcacggtctggcccgtgaggcgcgcgcagtcgtggatgctctatacgggcaaaaacgaaagcggtcagcggctcgactccgtggcctggaggctaagcgaacgggttgggctgcgcgtgtaccccggttcgaatctcgaatcaggctggagccgcagctaacgtggtactggcactcccgtctcgacccaagcctgcaccaaccctccaggatacggaggcgggtcgttttgcaactttttttggaggccggaaatgaaactagtaagcgcggaaagcggccgaccgcgatggctcgctgccgtagtctggagaagaatcgccagggttgcgttgcggtgtgccccggttcgaggccggccggattccgcggctaacgagggcgtggctgccccgtcgtttccaagaccccatagccagccgacttctccagttacggagcgagcccctcttttgttttgtttgtttttgccagatgcatcccgtactgcggcagatgcgcccccaccaccctccaccgcaacaacagccccctcctccacagccggcgcttctgcccccgccccagcagcagcagcaacttccagccacgaccgccgcggccgccgtgagcggggctggacagacttctcagtatgatcacctggccttggaagagggcgaggggctggcgcgcctgggggcgtcgtcgccggagcggcacccgcgcgtgcagatgaaaagggacgctcgcgaggcctacgtgcccaagcagaacctgttcagagacaggagcggcgaggagcccgaggagatgcgcgcggcccggttccacgcggggcgggagctgcggcgcggcctggaccgaaagagggtgctgagggacgaggatttcgaggcggacgagctgacggggatcagccccgcgcgcgcgcacgtggccgcggccaacctggtcacggcgtacgagcagaccgtgaaggaggagagcaacttccaaaaatccttcaacaaccacgtgcgcaccctgatcgcgcgcgaggaggtgaccctgggcctgatgcacctgtgggacctgctggaggccatcgtgcagaaccccaccagcaagccgctgacggcgcagctgttcctggtggtgcagcatagtcgggacaacgaggcgttcagggaggcgctgctgaatatcaccgagcccgagggccgctggctcctggacctggtgaacattctgcagagcatcgtggtgcaggagcgcgggctgccgctgtccgagaagctggcggccatcaacttctcggtgctgagtctgggcaagtactacgctaggaagatctacaagaccccgtacgtgcccatagacaaggaggtgaagatcgacgggttttacatgcgcatgaccctgaaagtgctgaccctgagcgacgatctgggggtgtaccgcaacgacaggatgcaccgcgcggtgagcgccagcaggcggcgcgagctgagcgaccaggagctgatgcacagcctgcagcgggccctgaccggggccgggaccgagggggagagctactttgacatgggcgcggacctgcactggcagcccagccgccgggccttggaggcggcaggcggtcccccctacatagaagaggtggacgatgaggtggacgaggagggcgagtacctggaagactgatggcgcgaccgtatttttgctagatgcaacaacagccacctcctgatcccgcgatgcgggcggcgctgcagagccagccgtccggcattaactcctcggacgattggacccaggccatgcaacgcatcatggcgctgacgacccgcaaccccgaagcctttagacagcagccccaggccaaccggctctcggccatcctggaggccgtggtgccctcgcgctccaaccccacgcacgagaaggtcctggccatcgtgaacgcgctggtggagaacaaggccatccgcggcgacgaggccggcctggtgtacaacgcgctgctggagcgcgtggcccgctacaacagcaccaacgtgcagaccaacctggaccgcatggtgaccgacgtgcgcgaggccgtggcccagcgcgagcggttccaccgcgagtccaacctgggatccatggtggcgctgaacgccttcctcagcacccagcccgccaacgtgccccggggccaggaggactacaccaacttcatcagcgccctgcgcctgatggtgaccgaggtgccccagagcgaggtgtaccagtccgggccggactacttcttccagaccagtcgccagggcttgcagaccgtgaacctgagccaggcgttcaagaacttgcagggcctgtggggcgtgcaggccccggtcggggaccgcgcgacggtgtcgagcctgctgacgccgaactcgcgcctgctgctgctgctggtggcccccttcacggacagcggcagcatcaaccgcaactcgtacctgggctacctgattaacctgtaccgcgaggccatcggccaggcgcacgtggacgagcagacctaccaggagatcacccacgtgagccgcgccctgggccaggacgacccgggcaatctggaagccaccctgaactttttgctgaccaaccggtcgcagaagatcccgccccagtacacgctcagcgccgaggaggagcgcatcctgcgatacgtgcagcagagcgtgggcctgttcctgatgcaggagggggccacccccagcgccgcgctcgacatgaccgcgcgcaacatggagcccagcatgtacgccagcaaccgcccgttcatcaataaactgatggactacttgcatcgggcggccgccatgaactctgactatttcaccaacgccatcctgaatccccactggctcccgccgccggggttctacacgggcgagtacgacatgcccgaccccaatgacgggttcctgtgggacgatgtggacagcagcgtgttctccccccgaccgggtgctaacgagcgccccttgtggaagaaggaaggcagcgaccgacgcccgtcctcggcgctgtccggccgcgagggtgctgccgcggcggtgcccgaggccgccagtcctttcccgagcttgcccttctcgctgaacagtattcgcagcagcgagctgggcaggatcacgcgcccgcgcttgctgggcgaggaggagtacttgaatgactcgctgttgagacccgagcgggagaagaacttccccaataacgggatagagagcctggtggacaagatgagccgctggaagacgtatgcgcaggagcacagggacgatccgtcgcagggggccacgagccggggcagcgccgcccgtaaacgccggtggcacgacaggcagcggggactgatgtgggacgatgaggattccgccgacgacagcagcgtgttggacttgggtgggagtggtaacccgttcgctcacctgcgcccccgcatcgggcgcatgatgtaagagaaaccgaaaataaatgatactcaccaaggccatggcgaccagcgtgcgttcgtttcttctctgttgttgtatctagtatgatgaggcgtgcgtacccggagggtcctcctccctcgtacgagagcgtgatgcagcaggcgatggcggcggcggcggcgatgcagcccccgctggaggctccttacgtgcccccgcggtacctggcgcctacggaggggcggaacagcattcgttactcggagctggcacccttgtacgataccacccggttgtacctggtggacaacaagtcggcggacatcgcctcgctgaactaccagaacgaccacagcaacttcctgaccaccgtggtgcagaacaatgacttcacccccacggaggccagcacccagaccatcaactttgacgagcgctcgcggtggggcggtcagctgaaaaccatcatgcacaccaacatgcccaacgtgaacgagttcatgtacagcaacaagttcaaggcgcgggtgatggtctcccgcaagacccccaacggggtgacagtgacagatggtagtcaggatatcttggagtatgaatgggtggagtttgagctgcccgaaggcaacttctcggtgaccatgaccatcgacctgatgaacaacgccatcatcgacaattacttggcggtggggcggcagaacggggtcctggagagcgatatcggcgtgaagttcgacactaggaacttcaggctgggctgggaccccgtgaccgagctggtcatgcccggggtgtacaccaacgaggccttccaccccgatattgtcttgctgcccggctgcggggtggacttcaccgagagccgcctcagcaacctgctgggcattcgcaagaggcagcccttccaggagggcttccagatcatgtacgaggatctggaggggggcaacatccccgcgctcctggatgtcgacgcctatgagaaaagcaaggaggagagcgccgccgcggcgactgcagctgtagccaccgcctctaccgaggtcaggggcgataattttgccagccctgcagcagtggcagcggccgaggcggctgaaaccgaaagtaagatagtcattcagccggtggagaaggatagcaaggacaggagctacaacgtgctgccggacaagataaacaccgcctaccgcagctggtacctggcctacaactatggcgaccccgagaagggcgtgcgctcctggacgctgctcaccacctcggacgtcacctgcggcgtggagcaagtctactggtcgctgcccgacatgatgcaagacccggtcaccttccgctccacgcgtcaagttagcaactacccggtggtgggcgccgagctcctgcccgtctactccaagagcttcttcaacgagcaggccgtctactcgcagcagctgcgcgccttcacctcgctcacgcacgtcttcaaccgcttccccgagaaccagatcctcgtccgcccgcccgcgcccaccattaccaccgtcagtgaaaacgttcctgctctcacagatcacgggaccctgccgctgcgcagcagtatccggggagtccagcgcgtgaccgttactgacgccagacgccgcacctgcccctacgtctacaaggccctgggcatagtcgcgccgcgcgtcctctcgagccgcaccttctaaaaaatgtccattctcatctcgcccagtaataacaccggttggggcctgcgcgcgcccagcaagatgtacggaggcgctcgccaacgctccacgcaacaccccgtgcgcgtgcgcgggcacttccgcgctccctggggcgccctcaagggccgcgtgcggtcgcgcaccaccgtcgacgacgtgatcgaccaggtggtggccgacgcgcgcaactacacccccgccgccgcgcccgtctccaccgtggacgccgtcatcgacagcgtggtggccgacgcgcgccggtacgcccgcgccaagagccggcggcggcgcatcgcccggcggcaccggagcacccccgccatgcgcgcggcgcgagccttgctgcgcagggccaggcgcacgggacgcagggccatgctcagggcggccagacgcgcggcttcaggcgccagcgccggcaggacccggagacgcgcggccacggcggcggcagcggccatcgccagcatgtcccgcccgcggcgagggaacgtgtactgggtgcgcgacgccgccaccggtgtgcgcgtgcccgtgcgcacccgcccccctcgcacttgaagatgttcacttcgcgatgttgatgtgtcccagcggcgaggaggatgtccaagcgcaaattcaaggaagagatgctccaggtcatcgcgcctgagatctacggccccgcggtggtgaaggaggaaagaaagccccgcaaaatcaagcgggtcaaaaaggacaaaaaggaagaagatgacgatctggtggagtttgtgcgcgagttcgccccccggcggcgcgtgcagtggcgcgggcggaaagtgcacccggtgctgagacccggcaccaccgtggtcttcacgcccggcgagcgctccggcagcgcttccaagcgctcctacgacgaggtgtacggggacgaggacatcctcgagcaggcggccgagcgcctgggcgagtttgcttacggcaagcgcagccgccccgccctgaaggaagaggcggtgtccatcccgctggaccacggcaaccccacgccgagcctcaagcccgtgaccctgcagcaggtgctgccgagcgcagcgccgcgccgggggttcaagcgcgagggcgaggatctgtaccccaccatgcagctgatggtgcccaagcgccagaagctggaagacgtgctggagaccatgaaggtggacccggacgtgcagcccgaggtcaaggtgcggcccatcaagcaggtggccccgggcctgggcgtgcagaccgtggacatcaagatccccacggagcccatggaaacgcagaccgagcccatgatcaagcccagcaccagcaccatggaggtgcagacggatccctggatgccatcggctcctagccgaagaccccggcgcaagtacggcgcggccagcctgctgatgcccaactacgcgctgcatccttccatcatccccacgccgggctaccgcggcacgcgcttctaccgcggtcatacaaccagccgccgccgcaagaccaccacccgccgccgccgtcgccgcacagccgctgcatctacccctgccgccctggtgcggagagtgtaccgccgcggccgcgcgcctctgaccctaccgcgcgcgcgctaccacccgagcatcgccatttaaactttcgcctgctttgcagatggccctcacatgccgcctccgcgttcccattacgggctaccgaggaagaaaaccgcgccgtagaaggctggcggggaacgggatgcgtcgccaccaccatcggcggcggcgcgccatcagcaagcggttggggggaggcttcctgcccgcgctgatccccatcatcgccgcggcgatcggggcgatccccggcattgcttccgtggcggtgcaggcctctcagcgccactgagacacttggaaaacatcttgtaataaaccaatggactctgacgctcctggtcctgtgatgtgttttcgtagacagatggaagacatcaatttttcgtccctggctccgcgacacggcacgcggccgttcatgggcacctggagcgacatcggcaccagccaactgaacgggggcgccttcaattggagcagtctctggagcgggcttaagaatttcgggtccacgcttaaaacctatggcagcaaggcgtggaacagcaccacagggcaggcgctgagggataagctgaaagagcagaacttccagcagaaggtggtcgatgggctcgcctcgggcatcaacggggtggtggacctggccaaccaggccgtgcagcggcagatcaacagccgcctggacccggtgccgcccgccggctccgtggagatgccgcaggtggaggaggagctgcctcccctggacaagcggggcgagaagcgaccccgccccgacgcggaggagacgctgctgacgcacacggacgagccgcccccgtacgaggaggcggtgaaactgggtctgcccaccacgcggcccatcgcgcccctggccaccggggtgctgaaacccgaaagtaataagcccgcgaccctggacttgcctcctcccgcttcccgcccctctacagtggctaagcccctgccgccggtggccgtggcccgcgcgcgacccgggggctccgcccgccctcatgcgaactggcagagcactctgaacagcatcgtgggtctgggagtgcagagtgtgaagcgccgccgctgctattaaacctaccgtagcgcttaacttgcttgtctgtgtgtgtatgtattatgtcgccgctgtccgccagaaggaggagtgaagaggcgcgtcgccgagttgcaagatggccaccccatcgatgctgccccagtgggcgtacatgcacatcgccggacaggacgcttcggagtacctgagtccgggtctggtgcagttcgcccgcgccacagacacctacttcagtctggggaacaagtttaggaaccccacggtggcgcccacgcacgatgtgaccaccgaccgcagccagcggctgacgctgcgcttcgtgcccgtggaccgcgaggacaacacctactcgtacaaagtgcgctacacgctggccgtgggcgacaaccgcgtgctggacatggccagcacctactttgacatccgcggcgtgctggatcggggccctagcttcaaaccctactccggcaccgcctacaacagcctggctcccaagggagcgcccaattccagccagtgggagcaaaaaaaggcaggcaatggtgacactatggaaacacacacatttggtgtggccccaatgggcggtgagaatattacaatcgacggattacaaattggaactgacgctacagctgatcaggataaaccaatttatgctgacaaaacattccagcctgaacctcaagtaggagaagaaaattggcaagaaactgaaagcttttatggcggtagggctcttaaaaaagacacaagcatgaaaccttgctatggctcctatgctagacccaccaatgtaaagggaggtcaagctaaacttaaagttggagctgatggagttcctaccaaagaatttgacatagacctggctttctttgatactcccggtggcacagtgaatggacaagatgagtataaagcagacattgtcatgtataccgaaaacacgtatctggaaactccagacacgcatgtggtatacaaaccaggcaaggatgatgcaagttctgaaattaacctggttcagcagtccatgcccaatagacccaactatattgggttcagagacaactttattgggctcatgtattacaacagtactggcaatatgggggtgctggctggtcaggcctcacagctgaatgctgtggtcgacttgcaagacagaaacaccgagctgtcataccagctcttgcttgactctttgggtgacagaacccggtatttcagtatgtggaatcaggcggtggacagttatgatcctgatgtgcgcattattgaaaaccatggtgtggaagacgaacttcccaactattgcttccccctggatgggtctggcactaatgccgcttaccaaggtgtgaaagtaaaaaatggtaacgatggtgatgttgagagcgaatgggaaaatgatgatactgtcgcagctcgaaatcaattatgcaagggcaacatttttgccatggaaattaacctccaagccaacctgtggagaagtttcctctactcgaacgtggccctgtacctgcccgactcttacaagtacacgccagccaacatcaccctgcccaccaacaccaacacttatgattacatgaacgggagagtggtgcctccctcgctggtggacgcctacatcaacatcggggcgcgctggtcgctggaccccatggacaacgtcaatcccttcaaccaccaccgcaacgcgggcctgcgctaccgctccatgctcctgggcaacgggcgctacgtgcccttccacatccaggtgccccagaaatttttcgccatcaagagcctcctgctcctgcccgggtcctacacctacgagtggaacttccgcaaggacgtcaacatgatcctgcagagctccctcggcaacgacctgcgcacggacggggcctccatctccttcaccagcatcaacctctacgccaccttcttccccatggcgcacaacacggcctccacgctcgaggccatgctgcgcaacgacaccaacgaccagtccttcaacgactacctctcggcggccaacatgctctaccccatcccggccaacgccaccaacgtgcccatctccatcccctcgcgcaactgggccgccttccgcggctggtccttcacgcgcctcaagaccaaggagacgccctcgctgggctccgggttcgacccctacttcgtctactcgggctccatcccctacctcgacggcaccttctacctcaaccacaccttcaagaaggtctccatcaccttcgactcctccgtcagctggcccggcaacgaccggctcctgacgcccaacgagttcgaaatcaagcgcaccgtcgacggcgagggatacaacgtggcccagtgcaacatgaccaaggactggttcctggtccagatgctggcccactacaacatcggctaccagggcttctacgtgcccgagggctacaaggaccgcatgtactccttcttccgcaacttccagcccatgagccgccaggtggtggacgaggtcaactacaaggactaccaggccgtcaccctggcctaccagcacaacaactcgggcttcgtcggctacctcgcgcccaccatgcgccagggccagccctaccccgccaactacccgtacccgctcatcggcaagagcgccgtcaccagcgtcacccagaaaaagttcctctgcgacagggtcatgtggcgcatccccttctccagcaacttcatgtccatgggcgcgctcaccgacctcggccagaacatgctctatgccaactccgcccacgcgctagacatgaatttcgaagtcgaccccatggatgagtccacccttctctatgttgtcttcgaagtcttcgacgtcgtccgagtgcaccagccccaccgcggcgtcatcgaggccgtctacctgcgcacccccttctcggccggtaacgccaccacctaaattgctacttgcatgatggctgagcccacaggctccggcgagcaggagctcagggccatcatccgcgacctgggctgcgggccctacttcctgggcaccttcgataagcgcttcccgggattcatggccccgcacaagctggcctgcgccatcgtcaacacggccggccgcgagaccgggggcgagcactggctggccttcgcctggaacccgcgctcgaacacctgctacctcttcgaccccttcgggttctcggacgagcgcctcaagcagatctaccagttcgagtacgagggcctgctgcgccgtagcgccctggccaccgaggaccgctgcgtcaccctggaaaagtccacccagaccgtgcagggtccgcgctcggccgcctgcgggctcttctgctgcatgttcctgcacgccttcgtgcactggcccgaccgccccatggacaagaaccccaccatgaacttgctgacgggggtgcccaacggcatgctccagtcgccccaggtggaacccaccctgcgccgcaaccaggaggcgctctaccgcttcctcaactcccactccgcctactttcgctcccaccgcgcgcgcatcgagaaggccaccgccttcgaccgcatgaacaatcaagacatgtaaaccgtgtgtgtatgtttaaaatatcttttaataaacagcactttaatgttacacatgcatctgagatgattttattttagaaatcgaaagggttctgccgggtctcggcatggcccgcgggcagggacacgttgcggaactggtacttggccagccacttgaactcggggatcagcagtttgggcagcggggtgtcggggaaggagtcggtccacagcttccgcgtcagctgcagggcgcccagcaggtcgggcgcggagatcttgaaatcgcagttgggacccgcgttctgcgcgcgagagttgcggtacacggggttgcagcactggaacaccatcagggccgggtgcttcacgctcgccagcaccgccgcgtcggtgatgctctccacgtcgaggtcctcggcgttggccatcccgaagggggtcatcttgcaggtctgccttcccatggtgggcacgcacccgggcttgtggttgcaatcgcagtgcagggggatcagcatcatctgggcctggtcggcgttcatccccgggtacatggccttcatgaaagcctccaattgcctgaacgcctgctgggccttggctccctcggtgaagaagaccccgcaggacttgctagagaactggttggtggcacagccggcatcgtgcacgcagcagcgcgcgtcgttgttggccagctgcaccacgctgcgcccccagcggttctgggtgatcttggcccggtcggggttctccttcagcgcgcgctgcccgttctcgctcgccacatccatctcgatcatgtgctccttctggatcatggtggtcccgtgcaggcaccgcagtttgccctcggcctcggtgcacccgtgcagccacagcgcgcacccggtgcactcccagttcttgtgggcgatctgggaatgcgcgtgcacgaacccttgcaggaagcggcccatcatggtcgtcagggtcttgttgctagtgaaggtcaacgggatgccgcggtgctcctcgttgatgtacaggtggcagatgcggcggtacacctcgccctgctcgggcatcagttggaagttggctttcaggtcggtctccacgcggtagcggtccatcagcatagtcatgatttccatgcccttctcccaggccgagacgatgggcaggctcatagggttcttcaccatcatcttagcactagcagccgcggccagggggtcgctctcatccagggtctcaaagctccgcttgccgtccttctcggtgatccgcaccggggggtagctgaagcccacggccgccagctcctcctcggcctgtctttcgtcctcgctgtcctggctgacgtcctgcatgaccacatgcttggtcttgcggggtttcttcttgggcggcagtggcggcggagatgcttgtggcgagggggagcgcgagttctcgctcaccactactatctcttcctcttcttggtccgaggccacgcggcggtaggtatgtctcttcgggggcagaggcggaggcgacgggctctcgccgccgcgacttggcggatggctggcagagccccttccgcgttcgggggtgcgctcccggcggcgctctgactgacttcctccgcggccggccattgtgttctcctagggaggaacaacaagcatggagactcagccatcgccaacctcgccatctgcccccaccgccggcgacgagaagcagcagcagcagaatgaaagcttaaccgccccgccgcccagccccgcctccgacgcagccgcggtcccagacatgcaagagatggaggaatccatcgagattgacctgggctatgtgacgcccgcggagcatgaggaggagctggcagtgcgctttcaatcgtcaagccaggaagataaagaacagccagagcaggaagcagagaacgagcagagtcaggctgggctcgagcatggcgactacctccacctgagcggggaggaggacgcgctcatcaagcatctggcccggcaggccaccatcgtcaaggacgcgctgctcgaccgcaccgaggtgcccctcagcgtggaggagctcagccgcgcctacgagctcaacctcttctcgccgcgcgtgccccccaagcgccagcccaacggcacctgcgagcccaacccccgcctcaacttctacccggtcttcgcggtgcccgaggccctggccacctaccacatctttttcaagaaccaaaagatccccgtctcctgccgcgccaaccgcacccgcgccgacgccctcttcaacctgggtcccggcgcccgcctacctgatatcgcctccttggaagaggttcccaagatcttcgagggtctgggcagcgacgagactcgggccgcgaacgctctgcaaggagaaggaggaggagagcatgagcaccacagcgccctggtcgagttggaaggcgacaacgcgcggctggcggtgctcaaacgcacggtcgagctgacccatttcgcctacccggctctgaacctgcccccgaaagtcatgagcgcggtcatggaccaggtgctcatcaagcgcgcgtcgcccatctccgaggacgagggcatgcaagactccgaggagggcaagcccgtggtcagcgacgagcagctggcccggtggctgggtcctaatgctacccctcaaagtttggaagagcggcgcaagctcatgatggccgtggtcctggtgaccgtggagctggagtgcctgcgccgcttcttcgccgacgcggagaccctgcgcaaggtcgaggagaacctgcactacctcttcaggcacgggttcgtgcgccaggcctgcaagatctccaacgtggagctgaccaacctggtctcctacatgggcatcttgcacgagaaccgcctggggcagaacgtgctgcacaccaccctgcgcggggaggcccgccgcgactacatccgcgactgcgtctacctctacctctgccacacctggcagacgggcatgggcgtgtggcagcagtgtctggaggagcagaacctgaaagagctctgcaagctcctgcaaaagaacctcaagggtctgtggaccgggttcgacgagcggaccaccgcctcggacctggccgacctcatcttccccgagcgcctcaggctgacgctgcgcaacggcctgcccgactttatgagccaaagcatgttgcaaaactttcgctctttcatcctcgaacgctccggaatcctgcccgccacctgctccgcgctgccctcggacttcgtgccgctgaccttccgcgagtgccccccgccgctgtggagccactgctacctgctgcgcctggccaactacctggcctaccactcggacgtgatcgaggacgtcagcggcgagggcctgctcgagtgccactgccgctgcaacctctgcacgccgcaccgctccctggcctgcaacccccagctgctgagcgagacccagatcatcggcaccttcgagttgcaagggcccagcgagggcgagggagccaaggggggtctgaaactcaccccggggctgtggacctcggcctacttgcgcaagttcgtgcccgaggattaccatcccttcgagatcaggttctacgaggaccaatcccagccgcccaaggccgagctgtcggcctgcgtcatcacccagggggcgatcctggcccaattgcaagccatccagaaatcccgccaagaattcttgctgaaaaagggccgcggggtctacctcgacccccagaccggtgaggagctcaaccccggcttcccccaggatgccccgaggaaacaagaagctgaaagtggagctgccgcccgtggaggatttggaggaagactgggagaacagcagtcaggcagaggagatggaggaagactgggacagcactcaggcagaggaggacagcctgcaagacagtctggaggaagacgaggaggaggcagaggaggaggtggaagaagcagccgccgccagaccgtcgtcctcggcgggggagaaagcaagcagcacggataccatctccgctccgggtcggggtcccgctcggccccacagtagatgggacgagaccgggcgattcccgaaccccaccacccagaccggtaagaaggagcggcagggatacaagtcctggcgggggcacaaaaacgccatcgtctcctgcttgcaggcctgcgggggcaacatctccttcacccggcgctacctgctcttccaccgcggggtgaacttcccccgcaacatcttgcattactaccgtcacctccacagcccctactacttccaagaagaggcagcagcagcagaaaaagaccagaaaaccagctagaaaatccacagcggcggcagcggcaggtggactgaggatcgcggcgaacgagccggcgcagacccgggagctgaggaaccggatctttcccaccctctatgccatcttccagcagagtcgggggcaggagcaggaactgaaagtcaagaaccgttctctgcgctcgctcacccgcagttgtctgtatcacaagagcgaagaccaacttcagcgcactctcgaggacgccgaggctctcttcaacaagtactgcgcgctcactcttaaagagtagcccgcgcccgcccagtcgcagaaaaaggcgggaattacgtcacctgtgcccttcgccctagccgcctccacccagcaccgccatgagcaaagagattcccacgccttacatgtggagctaccagccccagatgggcctggccgccggcgccgcccaggactactccacccgcatgaattggctcagcgccgggcccgcgatgatctcacgggtgaatgacatccgcgcccaccgaaaccagatactcctagaacagtcagcgctcaccgccacgccccgcaatcacctcaatccgcgtaattggcccgccgccctggtgtaccaggaaattccccagcccacgaccgtactacttccgcgagacgcccaggccgaagtccagctgactaactcaggtgtccagctggcgggcggcgccaccctgtgtcgtcaccgccccgctcagggtataaagcggctggtgatccggggcagaggcacacagctcaacgacgaggtggtgagctcttcgctgggtctgcgacctgacggagtcttccaactcgccggatcggggagatcttccttcacgcctcgtcaggcggtcctgactttggagagttcgtcctcgcagccccgctcgggcggcatcggcactctccagttcgtggaggagttcactccctcggtctacttcaaccccttctccggctcccccggccactacccggacgagttcatcccgaactttgacgccatcagcgagtcggtggacggctacgattgattaattaatcaactaaccccttacccctttaccctccagtaaaaataaagattaaaaatgattgaattgatcaataaagaatcacttacttgaaatctgaaaccaggtctctgtccatgttttctgtcagcagcacttcactcccctcttcccaactctggtactgcaggccccggcgggctgcaaacttcctccacactctgaaggggatgtcaaattcctcctgtccctcaatcttcatttttatcttctatcagatgtccaaaaagcgcgcgcgggtggatgatggcttcgaccccgtgtacccctacgatgcagacaacgcaccgactgtgcccttcatcaaccctcccttcgtctcttcagatggattccaagaaaagcccctgggggtgttgtccctgcgactggccgaccccgtcaccaccaagaatggggctgtcaccctcaagctgggggagggggtggacctcgacgactcgggaaaactcatctccaaaaatgccaccaaggccactgcccctctcagtatttccaacggcaccatttcccttaacatggctgcccctttttacaacaacaatggaacgttaagtctcaatgtttctacaccattagcagtatttcccacttttaacactttaggtatcagtcttggaaacggtcttcaaacttctaataagttgctgactgtacagttaactcatcctcttacattcagctcaaatagcatcacagtaaaaacagacaaaggactctatattaattctagtggaaacagagggcttgaggctaacataagcctaaaaagaggactgatttttgatggtaatgctattgcaacataccttggaagtggtttagactatggatcctatgatagcgatgggaaaacaagacccatcatcaccaaaattggagcaggtttgaattttgatgctaataatgccatggctgtgaagctaggcacaggtttaagttttgactctgccggtgccttaacagctggaaacaaagaggatgacaagctaacactttggactacacctgacccaagccctaattgtcaattactttcagacagagatgccaaatttaccctatgtcttacaaaatgcggtagtcaaatactaggcactgttgcagtagctgctgttactgtaggttcagcactaaatccaattaatgacacagtaaaaagcgccatagtattccttagatttgactctgacggtgtgctcatgtcaaactcatcaatggtaggtgattactggaactttagggaaggacagaccacccaaagtgtggcctatacaaatgctgtgggattcatgcccaatctaggtgcatatcctaaaacccaaagcaaaacaccaaaaaatagtatagtaagtcaggtatatttaaatggagaaactactatgccaatgacactgacaataactttcaatggcactgatgaaaaagacacaacacctgtgagcacttactccatgacttttacatggcagtggactggagactataaggacaagaatattacctttgctaccaactcctttactttctcctacatggcccaagaataaaccctgcatgccaaccccattgttcccaccactatggaaaactctgaagcagaaaaaaataaagttcaagtgttttattgattcaacagttttctcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgattaactttattccaaacggtctcggagcacttcaaaatgcaggtcccggaggtggcacctctcgcccccactgtgttggtggaaaataacagccaggtcaaaggtgacacggttctcgagatgttccacggtggcttccagcaaagcctccacgcgcacatccagaaacaagaggacagcgaaagcgggagcgttttctaattcctcaatcatcatattacactcctgcaccatccccagataattttcatttttccagccttgaatgattcgtattagttcctgaggtaaatccaagccagccatgataaaaagctcgcgcagagcgccctccaccggcattcttaagcacaccctcataattccaagagattctgctcctggttcacctgcagcagattaacaatgggaatatcaaaatctctgccgcgatccctaagctcctccctcaacaataactgtatgtaatctttcatatcatctccgaaatttttagccatagggccgccaggaataagagcagggcaagccacattacagataaagcgaagtcctccccagtgagcattgccaaatgtaagattgaaataagcatgctggctagaccctgtgatatcttccagataactggacagaaaatcaggcaagcaatttttaagaaaatcaacaaaagaaaagtcgtccaggtgcaggtttagagcctcaggaacaacgatggaataagtgcaaggagtgcgttccagcatggttagtgtttttttggtgatctgtagaacaaaaaataaacatgcaatattaaaccatgctagcctggcgaacaggtgggtaaatcactctttccagcaccaggcaggctacggggtctccggcgcgaccctcgtagaagctgtcgccatgattgaaaagcatcaccgagagaccttcccggtggccggcatggatgattcgagaagaagcatacactccgggaacattggcatccgtgagtgaaaaaaagcgacctataaagcctcggggcactacaatgctcaatctcaattccagcaaagccaccccatgcggatggagcacaaaattggcaggtgcgtaaaaaatgtaattactcccctcctgcacaggcagcaaagcccccgctccctccagaaacacatacaaagcctcagcgtccatagcttaccgagcacggcaggcgcaagagtcagagaaaaggctgagctctaacctgactgcccgctcctgtgctcaatatatagccctaacctacactgacgtaaaggccaaagtctaaaaatacccgccaaaatgacacacacgcccagcacacgcccagaaaccggtgacacactcaaaaaaatacgtgcgcttcctcaaacgcccaaaccggcgtcatttccgggttcccacgctacgtcaccgctcagcgactttcaaattccgtcgaccgttaaaaacgtcactcgccccgcccctaacggtcgcccttctctcggccaatcaccttcctcccttcccaaattcaaacgcctcatttgcatattaacgcgcacaaaaagtttgaggtatatatttgaatgatggtttaaac

SEQ ID NO: 16 - protein, 1273 aa, Unknown
  501Y.V2 - Brazil - Jenner C - wildtype
MFVFLVLLPLVSSQCVNFTNRTQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAAIKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 17 - protein, 1273 aa, Unknown
  501Y.V2 - Brazil - Jenner C - S2P
MFVFLVLLPLVSSQCVNFTNRTQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAAIKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 18 - protein, 1273 aa, Unknown
  501Y.V2 - Brazil - Jenner C - hexapro
MFVFLVLLPLVSSQCVNFTNRTQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAAIKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 19 - protein, 1270 aa, Unknown
  501Y.V3 - South Africa - Jenner D - wildtype
MFVFLVLLPLVSSQCVNLTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 20 - protein, 1270 aa, Unknown
  501Y.V3 - South Africa - Jenner D - S2P
MFVFLVLLPLVSSQCVNLTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 21 - protein, 1270 aa, Unknown
  501Y.V3 - South Africa - Jenner D - hexapro
MFVFLVLLPLVSSQCVNLTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 22 - protein, 1270 aa, Unknown
  501Y.V3 - South Africa - Jenner E.2 - S2P
MFVFLVLLPLVSSQCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 23 - DNA, 3906 bp, Unknown
  Nucleotide sequence coding for tPA-SARS CoV-2 spike protein E variant AZD2816 (SEQ ID NO: 3). tPA coding sequence = nt 1-96.
atggacgccatgaagcgaggcctgtgctgtgttctgcttctgtgtggcgccgtgtttgtgtccgccagccaagagatccacgccagatttcggagattcgtgttcctggtgctgctgccactggtgtctagccagtgtgtgaactttaccaccaggacacagctgcctccagcctacaccaacagcttcaccagaggcgtgtactacccagacaaggtgttcagatccagcgtgctgcactctacccaggacctgttcctgcctttcttcagcaacgtgacctggttccacgccatccacgtgtccggcaccaatggcaccaagagattcgccaacccagtgctgcccttcaacgacggagtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgacccattcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacacctatcaacctcgtgcggggcctgcctcagggcttctctgctctggaaccactggtggatctgcccatcggcatcaacatcacccggtttcagacactgcacagaagctacctgacacctggcgatagcagctctggatggacagctggcgccgctgcctactatgtgggatacctgcagcctcggaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttcccgaatatcaccaatctgtgcccattcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcacctaccaagctgaacgacctgtgcttcacaaacgtgtacgccgacagcttcgtgatccgtggagatgaagtgcggcagattgcacctggacagacaggcaatatcgccgactacaactacaagctgcccgacgacttcaccggctgtgtgattgcctggaacagcaacaacctggatagcaaagtcggcggcaactacaattacctgtaccggctgttccggaagtccaatctgaagcccttcgagcgggacatctccaccgagatctatcaggccggcagcacaccttgtaacggcgtgaaaggcttcaactgctacttcccactgcagtcctacggctttcagcccacatatggcgtgggctaccagccttacagagtggtggtgctgagcttcgagctgctgcatgctcctgccacagtgtgcggccctaagaaaagcaccaatctcgtgaagaacaaatgcgtgaacttcaacttcaacggcctgaccggcaccggcgtgctgacagagagcaacaagaagttcctgccattccagcagttcggccgggatatcgccgataccacagatgccgtcagagatcctcagacactggaaatcctggacatcacaccttgcagcttcggcggagtgtctgtgatcacgcctggcaccaacaccagcaatcaggtggcagtgctgtaccagggtgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccctatcggcgctggcatctgcgcctcttaccagacacagacaaacagccctagacgggccagatctgtggccagccagagcatcattgcctacacaatgtctctgggcgtggagaacagcgtggcctactccaacaactctatcgctatcccgaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacgcctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggcgctgccctgcagatcccatttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtctagcaacttcggcgccatcagctctgtgctgaacgatatcctgagcagactggacaaggtggaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcgcctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagcctcagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccctgacgtggacctgggcgatatcagcggaatcaatgccagcgtcgtgaacatccagaaagagatcgaccggctgaacgaggtggccaagaatctgaacgagagcctgatcgacctgcaagaactgggcaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggctcctgctgcaagttcgacgaggacgattctgagcccgtgctgaagggcgtgaaactgcactacacctga

SEQ ID NO: 24 - DNA, 3906 bp, Unknown
  Nucleotide sequence coding for tPA-SARS CoV-2 spike protein E6 variant in AZD3990 tPA coding sequence = nt 1-96.
atggacgccatgaagcgaggcctgtgctgtgttctgcttctgtgtggcgccgtgtttgtgtccgccagccaagagatccacgccagatttcggagattcgtgttcctggtgctgctgccactggtgtctagccagtgtgtgaactttaccaccaggacacagctgcctccagcctacaccaacagcttcaccagaggcgtgtactacccagacaaggtgttcagatccagcgtgctgcactctacccaggacctgttcctgcctttcttcagcaacgtgacctggttccacgccatccacgtgtccggcaccaatggcaccaagagattcgctaacccagtgctgcccttcaacgacggagtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgacccattcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacacctatcaacctcgtgcggggcctgcctcagggcttctctgctctggaaccactggtggatctgcccatcggcatcaacatcacccggtttcagacactgcacagaagctacctgacacctggcgatagcagctctggatggacagctggcgccgctgcctactatgtgggatacctgcagcctcggaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttcccgaatatcaccaatctgtgcccattcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcacctaccaagctgaacgacctgtgcttcacaaacgtgtacgccgacagcttcgtgatccgtggagatgaagtgcggcagattgcacctggacagacaggcaatatcgccgactacaactacaagctgcccgacgacttcaccggctgtgtgattgcctggaacagcaacaacctggatagcaaagtcggcggcaactacaattacctgtaccggctgttccggaagtccaatctgaagcccttcgagcgggacatctccaccgagatctatcaggccggcagcacaccttgtaacggcgtgaaaggcttcaactgctacttcccactgcagtcctacggctttcagcccacatatggcgtgggctaccagccttacagagtggtggtgctgagcttcgagctgctgcatgctcctgccacagtgtgcggccctaagaaaagcaccaatctcgtgaagaacaaatgcgtgaacttcaacttcaacggcctgaccggcaccggcgtgctgacagagagcaacaagaagttcctgccattccagcagttcggccgggatatcgccgataccacagatgccgtcagagatcctcagacactggaaatcctggacatcacaccttgcagcttcggcggagtgtctgtgatcacgcctggcaccaacaccagcaatcaggtggcagtgctgtaccagggtgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccctatcggcgctggcatctgcgcctcttaccagacacagacaaacagccctagacgggccagatctgtggccagccagagcatcattgcctacacaatgtctctgggcgtggagaacagcgtggcctactccaacaactctatcgctatcccgaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacgcctcctatcaaggacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgccctgcagatcccatttcctatgcagatggcctaccggttcaacggcatcggagtgacccagaatgtgctgtacgagaaccagaagctgatcgccaaccagttcaacagcgccatcggcaagatccaggacagcctgagcagcacacctagcgccctgggaaagctgcaggacgtggtcaaccagaatgcccaggcactgaacaccctggtcaagcagctgtctagcaacttcggcgccatcagctctgtgctgaacgatatcctgagcagactggaccctcctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcgcctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagcctcagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccctgacgtggacctgggcgatatcagcggaatcaatgccagcgtcgtgaacatccagaaagagatcgaccggctgaacgaggtggccaagaatctgaacgagagcctgatcgacctgcaagaactgggcaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggctcctgctgcaagttcgacgaggacgattctgagcccgtgctgaagggcgtgaaactgcactacacctga

SEQ ID NO: 25 - DNA, 35530 bp, Unknown
  AZD3990: Full Genome sequence AZD3990 (E6)
aaacccatcatcaataatatacctcaaactttttgtgcgcgttaatatgcaaatgaggcgtttgaatttgggaagggaggaaggtgattggccgagagaagggcgaccgttaggggcggggcgagtgacgttttgatgacgtgaccgcgaggaggagccagtttgcaagttctcgtgggaaaagtgacgtcaaacgaggtgtggtttgaacacggaaatactcaattttcccgcgctctctgacaggaaatgaggtgtttctaggcggatgcaagtgaaaacgggccattttcgcgcgaaaactgaatgaggaagtgaaaatctgagtaatttcgcgtttatgacagggaggagtatttgccgagggccgagtagactttgaccgattacgtgggggtttcgattaccgtgtttttcacctaaatttccgcgtacggtgtcaaagtccggtgtttttacgtaggtgtcagctgatcgccagggtatttaaacctgcgctctccagtcaagaggccactcttgagtgccagcgagaagagttttctcctccgcgcgcgagtcagatctacactttgaaaggcgatcgctagcgacatcgatcacaagtttgtacaaaaaagcaggctccaccatgggaacccgcgttttgagatttctgtcgccgactaaattcatgtcgcgcgatagtggtgtttatcgccgatagagatggcgatattggaaaaatcgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacgaagcttggtaccttaatggacgccatgaagcgaggcctgtgctgtgttctgcttctgtgtggcgccgtgtttgtgtccgccagccaagagatccacgccagatttcggagattcgtgttcctggtgctgctgccactggtgtctagccagtgtgtgaactttaccaccaggacacagctgcctccagcctacaccaacagcttcaccagaggcgtgtactacccagacaaggtgttcagatccagcgtgctgcactctacccaggacctgttcctgcctttcttcagcaacgtgacctggttccacgccatccacgtgtccggcaccaatggcaccaagagattcgctaacccagtgctgcccttcaacgacggagtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgacccattcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacacctatcaacctcgtgcggggcctgcctcagggcttctctgctctggaaccactggtggatctgcccatcggcatcaacatcacccggtttcagacactgcacagaagctacctgacacctggcgatagcagctctggatggacagctggcgccgctgcctactatgtgggatacctgcagcctcggaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttcccgaatatcaccaatctgtgcccattcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcacctaccaagctgaacgacctgtgcttcacaaacgtgtacgccgacagcttcgtgatccgtggagatgaagtgcggcagattgcacctggacagacaggcaatatcgccgactacaactacaagctgcccgacgacttcaccggctgtgtgattgcctggaacagcaacaacctggatagcaaagtcggcggcaactacaattacctgtaccggctgttccggaagtccaatctgaagcccttcgagcgggacatctccaccgagatctatcaggccggcagcacaccttgtaacggcgtgaaaggcttcaactgctacttcccactgcagtcctacggctttcagcccacatatggcgtgggctaccagccttacagagtggtggtgctgagcttcgagctgctgcatgctcctgccacagtgtgcggccctaagaaaagcaccaatctcgtgaagaacaaatgcgtgaacttcaacttcaacggcctgaccggcaccggcgtgctgacagagagcaacaagaagttcctgccattccagcagttcggccgggatatcgccgataccacagatgccgtcagagatcctcagacactggaaatcctggacatcacaccttgcagcttcggcggagtgtctgtgatcacgcctggcaccaacaccagcaatcaggtggcagtgctgtaccagggtgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccctatcggcgctggcatctgcgcctcttaccagacacagacaaacagccctagacgggccagatctgtggccagccagagcatcattgcctacacaatgtctctgggcgtggagaacagcgtggcctactccaacaactctatcgctatcccgaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacgcctcctatcaaggacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgccctgcagatcccatttcctatgcagatggcctaccggttcaacggcatcggagtgacccagaatgtgctgtacgagaaccagaagctgatcgccaaccagttcaacagcgccatcggcaagatccaggacagcctgagcagcacacctagcgccctgggaaagctgcaggacgtggtcaaccagaatgcccaggcactgaacaccctggtcaagcagctgtctagcaacttcggcgccatcagctctgtgctgaacgatatcctgagcagactggaccctcctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcgcctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagcctcagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccctgacgtggacctgggcgatatcagcggaatcaatgccagcgtcgtgaacatccagaaagagatcgaccggctgaacgaggtggccaagaatctgaacgagagcctgatcgacctgcaagaactgggcaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggctcctgctgcaagttcgacgaggacgattctgagcccgtgctgaagggcgtgaaactgcactacacctgataagcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgaggggggatcgatccgtcgagatatctagacccagctttcttgtacaaagtggtgatcgattcgacagatcgcgatcgcagtgagtagtgttctggggcgggggaggacctgcatgagggccagaatgactgaaatctgtgcttttctgtgtgttgcagcatcatgagcggaagcggctcctttgagggaggggtattcagcccttatctgacggggcgtctcccctcctgggcgggagtgcgtcagaatgtgatgggatccacggtggacggccggcccgtgcagcccgcgaactcttcaaccctgacctatgcaaccctgagctcttcgtcggtggacgcagctgccgccgcagctgctgcatccgccgccagcgccgtgcgcggaatggccatgggcgccggctactacggcactctggtggccaactcgagttccaccaataatcccgccagcctgaacgaggagaagctgctgctgctgatggcccagcttgaggccttgacccagcgcctgggcgagctgacccagcaggtggctcagctgcaggagcagacgcgggccgcggttgccacggtgaaatccaaataaaaaatgaatcaataaataaacggagacggttgttgattttaacacagagtctgaatctttatttgatttttcgcgcgcggtaggccctggaccaccggtctcgatcattgagcacccggtggatcttttccaggacccggtagaggtgggcttggatgttgaggtacatgggcatgagcccgtcccgggggtggaggtagctccattgcagggcctcgtgctcgggggtggtgttgtaaatcacccagtcatagcaggggcgcagggcgtggtgttgcacaatatctttgaggaggagactgatggccacgggcagccctttggtgtaggtgtttacaaatctgttgagctgggagggatgcatgcggggggagatgaggtgcatcttggcctggatcttgagattggcgatgttaccgcccagatcccgcctggggttcatgttgtgcaggaccaccagcacggtgtatccggtgcacttggggaatttatcatgcaacttggaagggaaggcgtgaaagaatttggcgacgcccttgtgtccgcccaggttttccatgcactcatccatgatgatggcaatgggcccgtgggcggcggcctgggcaaagacgtttcgggggtcggacacatcatagttgtggtcctgggtgaggtcatcataggccattttaatgaatttggggcggagggtgccggactgggggacaaaggtaccctcgatcccgggggcgtagttcccctcacagatctgcatctcccaggctttgagctcagagggggggatcatgtccacctgcggggcgataaagaacacggtttccggggcgggggagatgagctgggccgaaagcaagttccggagcagctgggacttgccgcagccggtggggccgtaaatgaccccgatgaccggctgcaggtggtagttgagggagagacagctgccgtcctcccggaggaggggggccacctcgttcatcatctcgcgcacgtgcatgttctcgcgcaccagttccgccaggaggcgctctccccccagagataggagctcctggagcgaggcgaagtttttcagcggcttgagtccgtcggccatgggcattttggagagggtctgttgcaagagttccaagcggtcccagagctcggtgatgtgctctacggcatctcgatccagcagacctcctcgtttcgcgggttgggacgactgcgggagtagggcaccagacgatgggcgtccagcgcagccagggtccggtccttccagggccgcagcgtccgcgtcagggtggtctccgtcacggtgaaggggtgcgcgccgggctgggcgcttgcgagggtgcgcttcaggctcatccggctggtcgaaaaccgctcccgatcggcgccctgcgcgtcggccaggtagcaattgaccatgagttcgtagttgagcgcctcggccgcgtggcctttggcgcggagcttacctttggaagtctgcccgcaggcgggacagaggagggacttgagggcgtagagcttgggggcgaggaagacggaatcgggggcgtaggcgtccgcgccgcagtgggcgcagacggtctcgcactccacgagccaggtgaggtcgggctggtcggggtcaaaaaccagtttcccgccgttctttttgatgcgtttcttacctttggtctccatgagctcgtgtccccgctgggtgacaaagaggctgtccgtgtccccgtagaccgactttatgggccggtcctcgagcggtgtgccgcggtcctcctcgtagaggaaccccgcccactccgagacgaaagcccgggtccaggccagcacgaaggaggccacgtgggacgggtagcggtcgttgtccaccagcgggtccactttttccagggtatgcaaacacatgtccccctcgtccacatccaggaaggtgattggcttgtaagtgtaggccacgtgaccgggggtcccggccgggggggtataaaagggggcgggcccctgctcgtcctcactgtcttccggatcgctgtccaggagcgccagctgttggggtaggtattccctctcgaaggcgggcatgacctcggcactcaggttgtcagtttctagaaacgaggaggatttgatattgacggtgccagcggagatgcctttcaagagcccctcgtccatctggtcagaaaagacgatttttttgttgtcgagcttggtggcgaaggagccgtagagggcgttggaaaggagcttggcgatggagcgcatggtctggtttttttccttgtcggcgcgctccttggccgcgatgttgagctgcacgtactcgcgcgccacgcacttccattcggggaagacggtggtcatctcgtcgggcacgattctgacctgccaacctcgattatgcagggtgatgaggtccacactggtggccacctcgccgcgcaggggctcgttggtccagcagaggcggccgcccttgcgcgagcagaaggggggcagagggtccagcatgacctcgtcgggggggtcggcatcgatggtgaagatgccgggcaggagatcggggtcgaagtagctgatggaagtggccagatcgtccagggaagcttgccattcgcgcacggccagcgcgcgctcgtagggactgaggggcgtgccccagggcatggggtgggtgagcgcggaggcgtacatgccgcagatgtcgtagacgtagaggggctcctcgaggatgccgatgtaggtggggtagcagcgccccccgcggatgctggcgcgcacgtagtcatacagctcgtgcgagggcgcgaggagccccgggcccaggttggtgcgactgggcttttcggcgcggtagacgatctggcgaaagatggcatgcgagttggaggagatggtgggcctttggaagatgttgaagtgggcgtgggggaggccgaccgagtcgcggatgaagtgggcgtaggagtcttgcagtttggcgacgagctcggcggtgacgaggacgtccagagcgcagtagtcgagggtctcctggatgatgtcatacttgagctggcccttttgtttccacagctcgcggttgagaaggaactcttcgcggtccttccagtactcttcgagggggaacccgtcctgatctgcacggtaagagcctagcatgtagaactggttgacggccttgtaggcgcagcagcccttctccacggggagggcgtaggcctgggcggccttgcgcagggaggtgtgcgtgagggcgaaggtgtccctgaccatgaccttgaggaactggtgcttgaaatcgatatcgtcgcagcccccctgctcccagagctggaagtccgtgcgcttcttgtaggcggggttgggcaaagcgaaagtaacatcgttgaaaaggatcttgcccgcgcggggcataaagttgcgagtgatgcggaaaggctggggcacctcggcccggttgttgatgacctgggcggcgagcacgatctcgtcgaaaccgttgatgttgtggcccacgatgtagagttccacgaatcgcgggcggcccttgacgtggggcagcttcttgagctcctcgtaggtgagctcgtcggggtcgctgagaccgtgctgctcgagcgcccagtcggcgagatgggggttggcgcggaggaaggaagtccagagatccacggccagggcggtttgcagacggtcccggtactgacggaactgctgcccgacggccattttttcgggggtgacgcagtagaaggtgcgggggtccccgtgccagcggtcccatttgagctggagggcgagatcgagggcgagctcgacgaggcggtcgtcccctgagagtttcatgaccagcatgaaggggacgagctgcttgccgaaggaccccatccaggtgtaggtttccacatcgtaggtgaggaagagcctttcggtgcgaggatgcgagccgatggggaagaactggatctcctgccaccaattggaggaatggctgttgatgtgatggaagtagaaatgccgacggcgcgccgaacactcgtgcttgtgtttatacaagcggccacagtgctcgcaacgctgcacgggatgcacgtgctgcacgagctgtacctgagttcctttgacgaggaatttcagtgggaagtggagtcgtggcgcctgcatctcgtgctgtactacgtcgtggtggtcggcctggccctcttctgcctcgatggtggtcatgctgacgagcccgcgcgggaggcaggtccagacctcggcgcgagcgggtcggagagcgaggacgagggcgcgcaggccggagctgtccagggtcctgagacgctgcggagtcaggtcagtgggcagcggcggcgcgcggttgacttgcaggagtttttccagggcgcgcgggaggtccagatggtacttgatctccaccgcgccgttggtggcgacgtcgatggcttgcagggtcccgtgcccctggggtgtgaccaccgtcccccgtttcttcttgggcggctggggcgacgggggcggtgcctcttccatggttagaagcggcggcgaggacgcgcgccgggcggcagaggcggctcggggcccggaggcaggggcggcaggggcacgtcggcgccgcgcgcgggtaggttctggtactgcgcccggagaagactggcgtgagcgacgacgcgacggttgacgtcctggatctgacgcctctgggtgaaggccacgggacccgtgagtttgaacctgaaagagagttcgacagaatcaatctcggtatcgttgacggcggcctgccgcaggatctcttgcacgtcgcccgagttgtcctggtaggcgatctcggtcatgaactgctcgatctcctcctcctgaaggtctccgcgaccggcgcgctccacggtggccgcgaggtcgttggagatgcggcccatgagctgcgagaaggcgttcatgcccgcctcgttccagacgcggctgtagaccacgacgccctcgggatcgcgggcgcgcatgaccacctgggcgaggttgagctccacgtggcgcgtgaagaccgcgtagttgcagaggcgctggtagaggtagttgagcgtggtggcgatgtgctcggtgacgaagaaatacatgatccagcggcggagcggcatctcgctgacgtcgcccagcgcctccaagcgttccatggcctcgtaaaagtccacggcgaagttgaaaaactgggagttgcgcgccgagacggtcaactcctcctccagaagacggatgagctcggcgatggtggcgcgcacctcgcgctcgaaggcccccgggagttcctccacttcctcctcttcttcctcctccactaacatctcttctacttcctcctcaggcggtggtggtggcgggggagggggcctgcgtcgccggcggcgcacgggcagacggtcgatgaagcgctcgatggtctcgccgcgccggcgtcgcatggtctcggtgacggcgcgcccgtcctcgcggggccgcagcgtgaagacgccgccgcgcatctccaggtggccgggggggtccccgttgggcagggagagggcgctgacgatgcatcttatcaattgccccgtagggactccgcgcaaggacctgagcgtctcgagatccacgggatctgaaaaccgttgaacgaaggcttcgagccagtcgcagtcgcaaggtaggctgagcacggtttcttctgccgggtcatgttggggagcggggcgggcgatgctgctggtgatgaagttgaaataggcggttctgagacggcggatggtggcgaggagcaccaggtctttgggcccggcttgctggatgcgcagacggtcggccatgccccaggcgtggtcctgacacctggccaggtccttgtagtagtcctgcatgagccgctccacgggcacctcctcctcgcccgcgcggccgtgcatgcgcgtgagcccgaagccgcgctggggctggacgagcgccaggtcggcgacgacgcgctcggcgaggatggcctgctggatctgggtgagggtggtctggaagtcgtcaaagtcgacgaagcggtggtaggctccggtgttgatggtgtaggagcagttggccatgacggaccagttgacggtctggtggcccggacgcacgagctcgtggtacttgaggcgcgagtaggcgcgcgtgtcgaagatgtagtcgttgcaggtgcgcaccaggtactggtagccgatgaggaagtgcggcggcggctggcggtagagcggccatcgctcggtggcgggggcgccgggcgcgaggtcctcgagcatggtgcggtggtagccgtagatgtacctggacatccaggtgatgccggcggcggtggtggaggcgcgcgggaactcgcggacgcggttccagatgttgcgcagcggcaggaagtagttcatggtgggcacggtctggcccgtgaggcgcgcgcagtcgtggatgctctatacgggcaaaaacgaaagcggtcagcggctcgactccgtggcctggaggctaagcgaacgggttgggctgcgcgtgtaccccggttcgaatctcgaatcaggctggagccgcagctaacgtggtactggcactcccgtctcgacccaagcctgcaccaaccctccaggatacggaggcgggtcgttttgcaactttttttggaggccggaaatgaaactagtaagcgcggaaagcggccgaccgcgatggctcgctgccgtagtctggagaagaatcgccagggttgcgttgcggtgtgccccggttcgaggccggccggattccgcggctaacgagggcgtggctgccccgtcgtttccaagaccccatagccagccgacttctccagttacggagcgagcccctcttttgttttgtttgtttttgccagatgcatcccgtactgcggcagatgcgcccccaccaccctccaccgcaacaacagccccctcctccacagccggcgcttctgcccccgccccagcagcagcagcaacttccagccacgaccgccgcggccgccgtgagcggggctggacagacttctcagtatgatcacctggccttggaagagggcgaggggctggcgcgcctgggggcgtcgtcgccggagcggcacccgcgcgtgcagatgaaaagggacgctcgcgaggcctacgtgcccaagcagaacctgttcagagacaggagcggcgaggagcccgaggagatgcgcgcggcccggttccacgcggggcgggagctgcggcgcggcctggaccgaaagagggtgctgagggacgaggatttcgaggcggacgagctgacggggatcagccccgcgcgcgcgcacgtggccgcggccaacctggtcacggcgtacgagcagaccgtgaaggaggagagcaacttccaaaaatccttcaacaaccacgtgcgcaccctgatcgcgcgcgaggaggtgaccctgggcctgatgcacctgtgggacctgctggaggccatcgtgcagaaccccaccagcaagccgctgacggcgcagctgttcctggtggtgcagcatagtcgggacaacgaggcgttcagggaggcgctgctgaatatcaccgagcccgagggccgctggctcctggacctggtgaacattctgcagagcatcgtggtgcaggagcgcgggctgccgctgtccgagaagctggcggccatcaacttctcggtgctgagtctgggcaagtactacgctaggaagatctacaagaccccgtacgtgcccatagacaaggaggtgaagatcgacgggttttacatgcgcatgaccctgaaagtgctgaccctgagcgacgatctgggggtgtaccgcaacgacaggatgcaccgcgcggtgagcgccagcaggcggcgcgagctgagcgaccaggagctgatgcacagcctgcagcgggccctgaccggggccgggaccgagggggagagctactttgacatgggcgcggacctgcactggcagcccagccgccgggccttggaggcggcaggcggtcccccctacatagaagaggtggacgatgaggtggacgaggagggcgagtacctggaagactgatggcgcgaccgtatttttgctagatgcaacaacagccacctcctgatcccgcgatgcgggcggcgctgcagagccagccgtccggcattaactcctcggacgattggacccaggccatgcaacgcatcatggcgctgacgacccgcaaccccgaagcctttagacagcagccccaggccaaccggctctcggccatcctggaggccgtggtgccctcgcgctccaaccccacgcacgagaaggtcctggccatcgtgaacgcgctggtggagaacaaggccatccgcggcgacgaggccggcctggtgtacaacgcgctgctggagcgcgtggcccgctacaacagcaccaacgtgcagaccaacctggaccgcatggtgaccgacgtgcgcgaggccgtggcccagcgcgagcggttccaccgcgagtccaacctgggatccatggtggcgctgaacgccttcctcagcacccagcccgccaacgtgccccggggccaggaggactacaccaacttcatcagcgccctgcgcctgatggtgaccgaggtgccccagagcgaggtgtaccagtccgggccggactacttcttccagaccagtcgccagggcttgcagaccgtgaacctgagccaggcgttcaagaacttgcagggcctgtggggcgtgcaggccccggtcggggaccgcgcgacggtgtcgagcctgctgacgccgaactcgcgcctgctgctgctgctggtggcccccttcacggacagcggcagcatcaaccgcaactcgtacctgggctacctgattaacctgtaccgcgaggccatcggccaggcgcacgtggacgagcagacctaccaggagatcacccacgtgagccgcgccctgggccaggacgacccgggcaatctggaagccaccctgaactttttgctgaccaaccggtcgcagaagatcccgccccagtacacgctcagcgccgaggaggagcgcatcctgcgatacgtgcagcagagcgtgggcctgttcctgatgcaggagggggccacccccagcgccgcgctcgacatgaccgcgcgcaacatggagcccagcatgtacgccagcaaccgcccgttcatcaataaactgatggactacttgcatcgggcggccgccatgaactctgactatttcaccaacgccatcctgaatccccactggctcccgccgccggggttctacacgggcgagtacgacatgcccgaccccaatgacgggttcctgtgggacgatgtggacagcagcgtgttctccccccgaccgggtgctaacgagcgccccttgtggaagaaggaaggcagcgaccgacgcccgtcctcggcgctgtccggccgcgagggtgctgccgcggcggtgcccgaggccgccagtcctttcccgagcttgcccttctcgctgaacagtattcgcagcagcgagctgggcaggatcacgcgcccgcgcttgctgggcgaggaggagtacttgaatgactcgctgttgagacccgagcgggagaagaacttccccaataacgggatagagagcctggtggacaagatgagccgctggaagacgtatgcgcaggagcacagggacgatccgtcgcagggggccacgagccggggcagcgccgcccgtaaacgccggtggcacgacaggcagcggggactgatgtgggacgatgaggattccgccgacgacagcagcgtgttggacttgggtgggagtggtaacccgttcgctcacctgcgcccccgcatcgggcgcatgatgtaagagaaaccgaaaataaatgatactcaccaaggccatggcgaccagcgtgcgttcgtttcttctctgttgttgtatctagtatgatgaggcgtgcgtacccggagggtcctcctccctcgtacgagagcgtgatgcagcaggcgatggcggcggcggcggcgatgcagcccccgctggaggctccttacgtgcccccgcggtacctggcgcctacggaggggcggaacagcattcgttactcggagctggcacccttgtacgataccacccggttgtacctggtggacaacaagtcggcggacatcgcctcgctgaactaccagaacgaccacagcaacttcctgaccaccgtggtgcagaacaatgacttcacccccacggaggccagcacccagaccatcaactttgacgagcgctcgcggtggggcggtcagctgaaaaccatcatgcacaccaacatgcccaacgtgaacgagttcatgtacagcaacaagttcaaggcgcgggtgatggtctcccgcaagacccccaacggggtgacagtgacagatggtagtcaggatatcttggagtatgaatgggtggagtttgagctgcccgaaggcaacttctcggtgaccatgaccatcgacctgatgaacaacgccatcatcgacaattacttggcggtggggcggcagaacggggtcctggagagcgatatcggcgtgaagttcgacactaggaacttcaggctgggctgggaccccgtgaccgagctggtcatgcccggggtgtacaccaacgaggccttccaccccgatattgtcttgctgcccggctgcggggtggacttcaccgagagccgcctcagcaacctgctgggcattcgcaagaggcagcccttccaggagggcttccagatcatgtacgaggatctggaggggggcaacatccccgcgctcctggatgtcgacgcctatgagaaaagcaaggaggagagcgccgccgcggcgactgcagctgtagccaccgcctctaccgaggtcaggggcgataattttgccagccctgcagcagtggcagcggccgaggcggctgaaaccgaaagtaagatagtcattcagccggtggagaaggatagcaaggacaggagctacaacgtgctgccggacaagataaacaccgcctaccgcagctggtacctggcctacaactatggcgaccccgagaagggcgtgcgctcctggacgctgctcaccacctcggacgtcacctgcggcgtggagcaagtctactggtcgctgcccgacatgatgcaagacccggtcaccttccgctccacgcgtcaagttagcaactacccggtggtgggcgccgagctcctgcccgtctactccaagagcttcttcaacgagcaggccgtctactcgcagcagctgcgcgccttcacctcgctcacgcacgtcttcaaccgcttccccgagaaccagatcctcgtccgcccgcccgcgcccaccattaccaccgtcagtgaaaacgttcctgctctcacagatcacgggaccctgccgctgcgcagcagtatccggggagtccagcgcgtgaccgttactgacgccagacgccgcacctgcccctacgtctacaaggccctgggcatagtcgcgccgcgcgtcctctcgagccgcaccttctaaaaaatgtccattctcatctcgcccagtaataacaccggttggggcctgcgcgcgcccagcaagatgtacggaggcgctcgccaacgctccacgcaacaccccgtgcgcgtgcgcgggcacttccgcgctccctggggcgccctcaagggccgcgtgcggtcgcgcaccaccgtcgacgacgtgatcgaccaggtggtggccgacgcgcgcaactacacccccgccgccgcgcccgtctccaccgtggacgccgtcatcgacagcgtggtggccgacgcgcgccggtacgcccgcgccaagagccggcggcggcgcatcgcccggcggcaccggagcacccccgccatgcgcgcggcgcgagccttgctgcgcagggccaggcgcacgggacgcagggccatgctcagggcggccagacgcgcggcttcaggcgccagcgccggcaggacccggagacgcgcggccacggcggcggcagcggccatcgccagcatgtcccgcccgcggcgagggaacgtgtactgggtgcgcgacgccgccaccggtgtgcgcgtgcccgtgcgcacccgcccccctcgcacttgaagatgttcacttcgcgatgttgatgtgtcccagcggcgaggaggatgtccaagcgcaaattcaaggaagagatgctccaggtcatcgcgcctgagatctacggccccgcggtggtgaaggaggaaagaaagccccgcaaaatcaagcgggtcaaaaaggacaaaaaggaagaagatgacgatctggtggagtttgtgcgcgagttcgccccccggcggcgcgtgcagtggcgcgggcggaaagtgcacccggtgctgagacccggcaccaccgtggtcttcacgcccggcgagcgctccggcagcgcttccaagcgctcctacgacgaggtgtacggggacgaggacatcctcgagcaggcggccgagcgcctgggcgagtttgcttacggcaagcgcagccgccccgccctgaaggaagaggcggtgtccatcccgctggaccacggcaaccccacgccgagcctcaagcccgtgaccctgcagcaggtgctgccgagcgcagcgccgcgccgggggttcaagcgcgagggcgaggatctgtaccccaccatgcagctgatggtgcccaagcgccagaagctggaagacgtgctggagaccatgaaggtggacccggacgtgcagcccgaggtcaaggtgcggcccatcaagcaggtggccccgggcctgggcgtgcagaccgtggacatcaagatccccacggagcccatggaaacgcagaccgagcccatgatcaagcccagcaccagcaccatggaggtgcagacggatccctggatgccatcggctcctagccgaagaccccggcgcaagtacggcgcggccagcctgctgatgcccaactacgcgctgcatccttccatcatccccacgccgggctaccgcggcacgcgcttctaccgcggtcatacaaccagccgccgccgcaagaccaccacccgccgccgccgtcgccgcacagccgctgcatctacccctgccgccctggtgcggagagtgtaccgccgcggccgcgcgcctctgaccctaccgcgcgcgcgctaccacccgagcatcgccatttaaactttcgcctgctttgcagatggccctcacatgccgcctccgcgttcccattacgggctaccgaggaagaaaaccgcgccgtagaaggctggcggggaacgggatgcgtcgccaccaccatcggcggcggcgcgccatcagcaagcggttggggggaggcttcctgcccgcgctgatccccatcatcgccgcggcgatcggggcgatccccggcattgcttccgtggcggtgcaggcctctcagcgccactgagacacttggaaaacatcttgtaataaaccaatggactctgacgctcctggtcctgtgatgtgttttcgtagacagatggaagacatcaatttttcgtccctggctccgcgacacggcacgcggccgttcatgggcacctggagcgacatcggcaccagccaactgaacgggggcgccttcaattggagcagtctctggagcgggcttaagaatttcgggtccacgcttaaaacctatggcagcaaggcgtggaacagcaccacagggcaggcgctgagggataagctgaaagagcagaacttccagcagaaggtggtcgatgggctcgcctcgggcatcaacggggtggtggacctggccaaccaggccgtgcagcggcagatcaacagccgcctggacccggtgccgcccgccggctccgtggagatgccgcaggtggaggaggagctgcctcccctggacaagcggggcgagaagcgaccccgccccgacgcggaggagacgctgctgacgcacacggacgagccgcccccgtacgaggaggcggtgaaactgggtctgcccaccacgcggcccatcgcgcccctggccaccggggtgctgaaacccgaaagtaataagcccgcgaccctggacttgcctcctcccgcttcccgcccctctacagtggctaagcccctgccgccggtggccgtggcccgcgcgcgacccgggggctccgcccgccctcatgcgaactggcagagcactctgaacagcatcgtgggtctgggagtgcagagtgtgaagcgccgccgctgctattaaacctaccgtagcgcttaacttgcttgtctgtgtgtgtatgtattatgtcgccgctgtccgccagaaggaggagtgaagaggcgcgtcgccgagttgcaagatggccaccccatcgatgctgccccagtgggcgtacatgcacatcgccggacaggacgcttcggagtacctgagtccgggtctggtgcagttcgcccgcgccacagacacctacttcagtctggggaacaagtttaggaaccccacggtggcgcccacgcacgatgtgaccaccgaccgcagccagcggctgacgctgcgcttcgtgcccgtggaccgcgaggacaacacctactcgtacaaagtgcgctacacgctggccgtgggcgacaaccgcgtgctggacatggccagcacctactttgacatccgcggcgtgctggatcggggccctagcttcaaaccctactccggcaccgcctacaacagcctggctcccaagggagcgcccaattccagccagtgggagcaaaaaaaggcaggcaatggtgacactatggaaacacacacatttggtgtggccccaatgggcggtgagaatattacaatcgacggattacaaattggaactgacgctacagctgatcaggataaaccaatttatgctgacaaaacattccagcctgaacctcaagtaggagaagaaaattggcaagaaactgaaagcttttatggcggtagggctcttaaaaaagacacaagcatgaaaccttgctatggctcctatgctagacccaccaatgtaaagggaggtcaagctaaacttaaagttggagctgatggagttcctaccaaagaatttgacatagacctggctttctttgatactcccggtggcacagtgaatggacaagatgagtataaagcagacattgtcatgtataccgaaaacacgtatctggaaactccagacacgcatgtggtatacaaaccaggcaaggatgatgcaagttctgaaattaacctggttcagcagtccatgcccaatagacccaactatattgggttcagagacaactttattgggctcatgtattacaacagtactggcaatatgggggtgctggctggtcaggcctcacagctgaatgctgtggtcgacttgcaagacagaaacaccgagctgtcataccagctcttgcttgactctttgggtgacagaacccggtatttcagtatgtggaatcaggcggtggacagttatgatcctgatgtgcgcattattgaaaaccatggtgtggaagacgaacttcccaactattgcttccccctggatgggtctggcactaatgccgcttaccaaggtgtgaaagtaaaaaatggtaacgatggtgatgttgagagcgaatgggaaaatgatgatactgtcgcagctcgaaatcaattatgcaagggcaacatttttgccatggaaattaacctccaagccaacctgtggagaagtttcctctactcgaacgtggccctgtacctgcccgactcttacaagtacacgccagccaacatcaccctgcccaccaacaccaacacttatgattacatgaacgggagagtggtgcctccctcgctggtggacgcctacatcaacatcggggcgcgctggtcgctggaccccatggacaacgtcaatcccttcaaccaccaccgcaacgcgggcctgcgctaccgctccatgctcctgggcaacgggcgctacgtgcccttccacatccaggtgccccagaaatttttcgccatcaagagcctcctgctcctgcccgggtcctacacctacgagtggaacttccgcaaggacgtcaacatgatcctgcagagctccctcggcaacgacctgcgcacggacggggcctccatctccttcaccagcatcaacctctacgccaccttcttccccatggcgcacaacacggcctccacgctcgaggccatgctgcgcaacgacaccaacgaccagtccttcaacgactacctctcggcggccaacatgctctaccccatcccggccaacgccaccaacgtgcccatctccatcccctcgcgcaactgggccgccttccgcggctggtccttcacgcgcctcaagaccaaggagacgccctcgctgggctccgggttcgacccctacttcgtctactcgggctccatcccctacctcgacggcaccttctacctcaaccacaccttcaagaaggtctccatcaccttcgactcctccgtcagctggcccggcaacgaccggctcctgacgcccaacgagttcgaaatcaagcgcaccgtcgacggcgagggatacaacgtggcccagtgcaacatgaccaaggactggttcctggtccagatgctggcccactacaacatcggctaccagggcttctacgtgcccgagggctacaaggaccgcatgtactccttcttccgcaacttccagcccatgagccgccaggtggtggacgaggtcaactacaaggactaccaggccgtcaccctggcctaccagcacaacaactcgggcttcgtcggctacctcgcgcccaccatgcgccagggccagccctaccccgccaactacccgtacccgctcatcggcaagagcgccgtcaccagcgtcacccagaaaaagttcctctgcgacagggtcatgtggcgcatccccttctccagcaacttcatgtccatgggcgcgctcaccgacctcggccagaacatgctctatgccaactccgcccacgcgctagacatgaatttcgaagtcgaccccatggatgagtccacccttctctatgttgtcttcgaagtcttcgacgtcgtccgagtgcaccagccccaccgcggcgtcatcgaggccgtctacctgcgcacccccttctcggccggtaacgccaccacctaaattgctacttgcatgatggctgagcccacaggctccggcgagcaggagctcagggccatcatccgcgacctgggctgcgggccctacttcctgggcaccttcgataagcgcttcccgggattcatggccccgcacaagctggcctgcgccatcgtcaacacggccggccgcgagaccgggggcgagcactggctggccttcgcctggaacccgcgctcgaacacctgctacctcttcgaccccttcgggttctcggacgagcgcctcaagcagatctaccagttcgagtacgagggcctgctgcgccgtagcgccctggccaccgaggaccgctgcgtcaccctggaaaagtccacccagaccgtgcagggtccgcgctcggccgcctgcgggctcttctgctgcatgttcctgcacgccttcgtgcactggcccgaccgccccatggacaagaaccccaccatgaacttgctgacgggggtgcccaacggcatgctccagtcgccccaggtggaacccaccctgcgccgcaaccaggaggcgctctaccgcttcctcaactcccactccgcctactttcgctcccaccgcgcgcgcatcgagaaggccaccgccttcgaccgcatgaacaatcaagacatgtaaaccgtgtgtgtatgtttaaaatatcttttaataaacagcactttaatgttacacatgcatctgagatgattttattttagaaatcgaaagggttctgccgggtctcggcatggcccgcgggcagggacacgttgcggaactggtacttggccagccacttgaactcggggatcagcagtttgggcagcggggtgtcggggaaggagtcggtccacagcttccgcgtcagctgcagggcgcccagcaggtcgggcgcggagatcttgaaatcgcagttgggacccgcgttctgcgcgcgagagttgcggtacacggggttgcagcactggaacaccatcagggccgggtgcttcacgctcgccagcaccgccgcgtcggtgatgctctccacgtcgaggtcctcggcgttggccatcccgaagggggtcatcttgcaggtctgccttcccatggtgggcacgcacccgggcttgtggttgcaatcgcagtgcagggggatcagcatcatctgggcctggtcggcgttcatccccgggtacatggccttcatgaaagcctccaattgcctgaacgcctgctgggccttggctccctcggtgaagaagaccccgcaggacttgctagagaactggttggtggcacagccggcatcgtgcacgcagcagcgcgcgtcgttgttggccagctgcaccacgctgcgcccccagcggttctgggtgatcttggcccggtcggggttctccttcagcgcgcgctgcccgttctcgctcgccacatccatctcgatcatgtgctccttctggatcatggtggtcccgtgcaggcaccgcagtttgccctcggcctcggtgcacccgtgcagccacagcgcgcacccggtgcactcccagttcttgtgggcgatctgggaatgcgcgtgcacgaacccttgcaggaagcggcccatcatggtcgtcagggtcttgttgctagtgaaggtcaacgggatgccgcggtgctcctcgttgatgtacaggtggcagatgcggcggtacacctcgccctgctcgggcatcagttggaagttggctttcaggtcggtctccacgcggtagcggtccatcagcatagtcatgatttccatgcccttctcccaggccgagacgatgggcaggctcatagggttcttcaccatcatcttagcactagcagccgcggccagggggtcgctctcatccagggtctcaaagctccgcttgccgtccttctcggtgatccgcaccggggggtagctgaagcccacggccgccagctcctcctcggcctgtctttcgtcctcgctgtcctggctgacgtcctgcatgaccacatgcttggtcttgcggggtttcttcttgggcggcagtggcggcggagatgcttgtggcgagggggagcgcgagttctcgctcaccactactatctcttcctcttcttggtccgaggccacgcggcggtaggtatgtctcttcgggggcagaggcggaggcgacgggctctcgccgccgcgacttggcggatggctggcagagccccttccgcgttcgggggtgcgctcccggcggcgctctgactgacttcctccgcggccggccattgtgttctcctagggaggaacaacaagcatggagactcagccatcgccaacctcgccatctgcccccaccgccggcgacgagaagcagcagcagcagaatgaaagcttaaccgccccgccgcccagccccgcctccgacgcagccgcggtcccagacatgcaagagatggaggaatccatcgagattgacctgggctatgtgacgcccgcggagcatgaggaggagctggcagtgcgctttcaatcgtcaagccaggaagataaagaacagccagagcaggaagcagagaacgagcagagtcaggctgggctcgagcatggcgactacctccacctgagcggggaggaggacgcgctcatcaagcatctggcccggcaggccaccatcgtcaaggacgcgctgctcgaccgcaccgaggtgcccctcagcgtggaggagctcagccgcgcctacgagctcaacctcttctcgccgcgcgtgccccccaagcgccagcccaacggcacctgcgagcccaacccccgcctcaacttctacccggtcttcgcggtgcccgaggccctggccacctaccacatctttttcaagaaccaaaagatccccgtctcctgccgcgccaaccgcacccgcgccgacgccctcttcaacctgggtcccggcgcccgcctacctgatatcgcctccttggaagaggttcccaagatcttcgagggtctgggcagcgacgagactcgggccgcgaacgctctgcaaggagaaggaggaggagagcatgagcaccacagcgccctggtcgagttggaaggcgacaacgcgcggctggcggtgctcaaacgcacggtcgagctgacccatttcgcctacccggctctgaacctgcccccgaaagtcatgagcgcggtcatggaccaggtgctcatcaagcgcgcgtcgcccatctccgaggacgagggcatgcaagactccgaggagggcaagcccgtggtcagcgacgagcagctggcccggtggctgggtcctaatgctacccctcaaagtttggaagagcggcgcaagctcatgatggccgtggtcctggtgaccgtggagctggagtgcctgcgccgcttcttcgccgacgcggagaccctgcgcaaggtcgaggagaacctgcactacctcttcaggcacgggttcgtgcgccaggcctgcaagatctccaacgtggagctgaccaacctggtctcctacatgggcatcttgcacgagaaccgcctggggcagaacgtgctgcacaccaccctgcgcggggaggcccgccgcgactacatccgcgactgcgtctacctctacctctgccacacctggcagacgggcatgggcgtgtggcagcagtgtctggaggagcagaacctgaaagagctctgcaagctcctgcaaaagaacctcaagggtctgtggaccgggttcgacgagcggaccaccgcctcggacctggccgacctcatcttccccgagcgcctcaggctgacgctgcgcaacggcctgcccgactttatgagccaaagcatgttgcaaaactttcgctctttcatcctcgaacgctccggaatcctgcccgccacctgctccgcgctgccctcggacttcgtgccgctgaccttccgcgagtgccccccgccgctgtggagccactgctacctgctgcgcctggccaactacctggcctaccactcggacgtgatcgaggacgtcagcggcgagggcctgctcgagtgccactgccgctgcaacctctgcacgccgcaccgctccctggcctgcaacccccagctgctgagcgagacccagatcatcggcaccttcgagttgcaagggcccagcgagggcgagggagccaaggggggtctgaaactcaccccggggctgtggacctcggcctacttgcgcaagttcgtgcccgaggattaccatcccttcgagatcaggttctacgaggaccaatcccagccgcccaaggccgagctgtcggcctgcgtcatcacccagggggcgatcctggcccaattgcaagccatccagaaatcccgccaagaattcttgctgaaaaagggccgcggggtctacctcgacccccagaccggtgaggagctcaaccccggcttcccccaggatgccccgaggaaacaagaagctgaaagtggagctgccgcccgtggaggatttggaggaagactgggagaacagcagtcaggcagaggagatggaggaagactgggacagcactcaggcagaggaggacagcctgcaagacagtctggaggaagacgaggaggaggcagaggaggaggtggaagaagcagccgccgccagaccgtcgtcctcggcgggggagaaagcaagcagcacggataccatctccgctccgggtcggggtcccgctcggccccacagtagatgggacgagaccgggcgattcccgaaccccaccacccagaccggtaagaaggagcggcagggatacaagtcctggcgggggcacaaaaacgccatcgtctcctgcttgcaggcctgcgggggcaacatctccttcacccggcgctacctgctcttccaccgcggggtgaacttcccccgcaacatcttgcattactaccgtcacctccacagcccctactacttccaagaagaggcagcagcagcagaaaaagaccagaaaaccagctagaaaatccacagcggcggcagcggcaggtggactgaggatcgcggcgaacgagccggcgcagacccgggagctgaggaaccggatctttcccaccctctatgccatcttccagcagagtcgggggcaggagcaggaactgaaagtcaagaaccgttctctgcgctcgctcacccgcagttgtctgtatcacaagagcgaagaccaacttcagcgcactctcgaggacgccgaggctctcttcaacaagtactgcgcgctcactcttaaagagtagcccgcgcccgcccagtcgcagaaaaaggcgggaattacgtcacctgtgcccttcgccctagccgcctccacccagcaccgccatgagcaaagagattcccacgccttacatgtggagctaccagccccagatgggcctggccgccggcgccgcccaggactactccacccgcatgaattggctcagcgccgggcccgcgatgatctcacgggtgaatgacatccgcgcccaccgaaaccagatactcctagaacagtcagcgctcaccgccacgccccgcaatcacctcaatccgcgtaattggcccgccgccctggtgtaccaggaaattccccagcccacgaccgtactacttccgcgagacgcccaggccgaagtccagctgactaactcaggtgtccagctggcgggcggcgccaccctgtgtcgtcaccgccccgctcagggtataaagcggctggtgatccggggcagaggcacacagctcaacgacgaggtggtgagctcttcgctgggtctgcgacctgacggagtcttccaactcgccggatcggggagatcttccttcacgcctcgtcaggcggtcctgactttggagagttcgtcctcgcagccccgctcgggcggcatcggcactctccagttcgtggaggagttcactccctcggtctacttcaaccccttctccggctcccccggccactacccggacgagttcatcccgaactttgacgccatcagcgagtcggtggacggctacgattgattaattaatcaactaaccccttacccctttaccctccagtaaaaataaagattaaaaatgattgaattgatcaataaagaatcacttacttgaaatctgaaaccaggtctctgtccatgttttctgtcagcagcacttcactcccctcttcccaactctggtactgcaggccccggcgggctgcaaacttcctccacactctgaaggggatgtcaaattcctcctgtccctcaatcttcatttttatcttctatcagatgtccaaaaagcgcgcgcgggtggatgatggcttcgaccccgtgtacccctacgatgcagacaacgcaccgactgtgcccttcatcaaccctcccttcgtctcttcagatggattccaagaaaagcccctgggggtgttgtccctgcgactggccgaccccgtcaccaccaagaatggggctgtcaccctcaagctgggggagggggtggacctcgacgactcgggaaaactcatctccaaaaatgccaccaaggccactgcccctctcagtatttccaacggcaccatttcccttaacatggctgcccctttttacaacaacaatggaacgttaagtctcaatgtttctacaccattagcagtatttcccacttttaacactttaggtatcagtcttggaaacggtcttcaaacttctaataagttgctgactgtacagttaactcatcctcttacattcagctcaaatagcatcacagtaaaaacagacaaaggactctatattaattctagtggaaacagagggcttgaggctaacataagcctaaaaagaggactgatttttgatggtaatgctattgcaacataccttggaagtggtttagactatggatcctatgatagcgatgggaaaacaagacccatcatcaccaaaattggagcaggtttgaattttgatgctaataatgccatggctgtgaagctaggcacaggtttaagttttgactctgccggtgccttaacagctggaaacaaagaggatgacaagctaacactttggactacacctgacccaagccctaattgtcaattactttcagacagagatgccaaatttaccctatgtcttacaaaatgcggtagtcaaatactaggcactgttgcagtagctgctgttactgtaggttcagcactaaatccaattaatgacacagtaaaaagcgccatagtattccttagatttgactctgacggtgtgctcatgtcaaactcatcaatggtaggtgattactggaactttagggaaggacagaccacccaaagtgtggcctatacaaatgctgtgggattcatgcccaatctaggtgcatatcctaaaacccaaagcaaaacaccaaaaaatagtatagtaagtcaggtatatttaaatggagaaactactatgccaatgacactgacaataactttcaatggcactgatgaaaaagacacaacacctgtgagcacttactccatgacttttacatggcagtggactggagactataaggacaagaatattacctttgctaccaactcctttactttctcctacatggcccaagaataaaccctgcatgccaaccccattgttcccaccactatggaaaactctgaagcagaaaaaaataaagttcaagtgttttattgattcaacagttttctcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgattaactttattccaaacggtctcggagcacttcaaaatgcaggtcccggaggtggcacctctcgcccccactgtgttggtggaaaataacagccaggtcaaaggtgacacggttctcgagatgttccacggtggcttccagcaaagcctccacgcgcacatccagaaacaagaggacagcgaaagcgggagcgttttctaattcctcaatcatcatattacactcctgcaccatccccagataattttcatttttccagccttgaatgattcgtattagttcctgaggtaaatccaagccagccatgataaaaagctcgcgcagagcgccctccaccggcattcttaagcacaccctcataattccaagagattctgctcctggttcacctgcagcagattaacaatgggaatatcaaaatctctgccgcgatccctaagctcctccctcaacaataactgtatgtaatctttcatatcatctccgaaatttttagccatagggccgccaggaataagagcagggcaagccacattacagataaagcgaagtcctccccagtgagcattgccaaatgtaagattgaaataagcatgctggctagaccctgtgatatcttccagataactggacagaaaatcaggcaagcaatttttaagaaaatcaacaaaagaaaagtcgtccaggtgcaggtttagagcctcaggaacaacgatggaataagtgcaaggagtgcgttccagcatggttagtgtttttttggtgatctgtagaacaaaaaataaacatgcaatattaaaccatgctagcctggcgaacaggtgggtaaatcactctttccagcaccaggcaggctacggggtctccggcgcgaccctcgtagaagctgtcgccatgattgaaaagcatcaccgagagaccttcccggtggccggcatggatgattcgagaagaagcatacactccgggaacattggcatccgtgagtgaaaaaaagcgacctataaagcctcggggcactacaatgctcaatctcaattccagcaaagccaccccatgcggatggagcacaaaattggcaggtgcgtaaaaaatgtaattactcccctcctgcacaggcagcaaagcccccgctccctccagaaacacatacaaagcctcagcgtccatagcttaccgagcacggcaggcgcaagagtcagagaaaaggctgagctctaacctgactgcccgctcctgtgctcaatatatagccctaacctacactgacgtaaaggccaaagtctaaaaatacccgccaaaatgacacacacgcccagcacacgcccagaaaccggtgacacactcaaaaaaatacgtgcgcttcctcaaacgcccaaaccggcgtcatttccgggttcccacgctacgtcaccgctcagcgactttcaaattccgtcgaccgttaaaaacgtcactcgccccgcccctaacggtcgcccttctctcggccaatcaccttcctcccttcccaaattcaaacgcctcatttgcatattaacgcgcacaaaaagtttgaggtatatatttgaatgatggttt

(19) United States
(12) Patent Application Publication
(10) Pub. No.: US 2023/0285532 A1
(43) Pub. Date: Sep. 14, 2023

(54) Compositions and Methods for Inducing an Immune Response

(71) Applicant: Oxford University Innovation Limited, Oxford (GB)

(72) Inventors: Sarah C. GILBERT, Oxford (GB); Teresa LAMBE, Oxford (GB); Sarah SEBASTIAN, Oxford (GB)

(21) Appl. No.: 17/200,297

(22) Filed: Mar. 12, 2021

(30) Foreign Application Priority Data
Mar. 13, 2020 (GB) 2003670.3
May 5, 2020 (GB) 2006608.0
May 13, 2020 (GB) 2007062.9
Jun. 17, 2020 (GB) 2009239.1
Jul. 9, 2020 (GB) 2010569.8
Oct. 26, 2020 (GB) 2016922.3
Oct. 30, 2020 (GB) 2017284.7
Nov. 9, 2020 (GB) 2017677.2
Nov. 23, 2020 (GB) 2018410.7
Nov. 27, 2020 (GB) 2018718.3
Jan. 4, 2021 (GB) 2100034.4

(51) Int. Cl. A61K 39/12; A61P 31/14; C12N 15/86

(52) U.S. Cl. CPC A61K 39/12; A61P 31/14; C12N 15/86; A61K 2039/54

(57) ABSTRACT

The invention relates to a composition comprising a viral vector, the viral vector comprising nucleic acid having a polynucleotide sequence encoding the spike protein from the coronavirus SARS-CoV2, characterised in that said viral vector is an adenovirus based vector. The invention also relates to uses of such a composition and methods of treatment.

### CROSS-REFERENCE TO RELATED APPLICATIONS

This application claims priority to United Kingdom Patent Application No. GB2003670.3 having an international filing date of Mar. 13, 2020, United Kingdom Patent Application No. GB2006608.0 having an international filing date of May 5, 2020, United Kingdom Patent Application No. GB2007062.9 having an international filing date of May 13, 2020, United Kingdom Patent Application No. GB2009239.1 having an international filing date of Jun. 17, 2020, United Kingdom Patent Application No. GB2010569.8 having an international filing date of Jul. 9, 2020, United Kingdom Patent Application No. GB2016922.3 having an international filing date of Oct. 26, 2020, United Kingdom Patent Application No. GB2017284.7 having an international filing date of Oct. 30, 2020, United Kingdom Patent Application No. GB2017677.2 having an international filing date of Nov. 9, 2020, United Kingdom Patent Application No. GB2018410.7 having an international filing date of Nov. 23, 2020, United Kingdom Patent Application No. GB2018718.3 having an international filing date of Nov. 27, 2020, and United Kingdom Patent Application No. GB2100034.4 having an international filing date of Jan. 4, 2021, which are incorporated herein by reference.

### FIELD OF THE INVENTION

The invention relates to induction of immune responses, suitably protective immune responses, against SARS-CoV2 (nC0V-19).

### INCORPORATION-BY-REFERENCE OF SEQUENCE LISTING OR TABLE

The material in the accompanying sequence listing is hereby incorporated by reference in its entirety into this application. The accompanying file, named Sequence Listing 23049_0039_ST25.txt was created on Oct. 25, 2021 and is 377 KB. The file can be accessed using Microsoft Word on a computer that uses Windows OS.

### BACKGROUND TO THE INVENTION

Coronavirus 19 (SARS-CoV2; sometimes referred to as nCoV-19 or as COVID-19) is the virus responsible for an outbreak of coronavirus disease that was first reported from Wuhan, China, on 31 Dec. 2019.

Symptoms of the disease include fever, dry cough, muscle pain, and respiratory problems such as breathing difficulties / shortness of breath. In more severe cases, infection can cause pneumonia, severe acute respiratory syndrome, kidney failure and even death. Mortality rates have been estimated by the World Health Organisation (WHO) at up to 3.4% of infected individuals, with many commentators agreeing on a mortality rate of approx. 1-2% of infected individuals once figures are adjusted taking into account the mildest cases which are not always reported (e.g. if individuals did not seek treatment or diagnosis).

According to the World Health Organisation report as of 7 Mar. 2020, the global number of confirmed cases of COVID-19 had surpassed 100,000.

No licensed vaccines or treatments are currently available for SARS-CoV2 infections. Ongoing disease control strategies have so far relied on minimising contact with infected individuals, observing standard infection control measures to limit nosocomial transmission, contact tracing and quarantine. Quarantine measures affecting millions of people have been implemented by numerous countries including at the origin of the outbreak in China. European countries such as Italy have quarantined up to 16 m people - more than a quarter of the population.

The health and economic impacts of the numerous epidemics has been serious and continue to escalate. The World Health Organisation report of 11 Mar. 2020 characterised the outbreak as a pandemic. This is the first ever pandemic caused by a coronavirus. The absence of any specific treatment is exacerbating the economic impact and more importantly is a problem leading to loss of human lives. The lack of any vaccine against this virus is a serious problem in the art.

The logistical effects of large scale changes in behaviour as a response to the SARS-CoV2 outbreak are significant and are causing secondary risks to health. For example, on 9 Mar. 2020 the UK government’s Department for Environment, Food & Rural Affairs (DEFRA) was forced to relax their enforcement of restrictions on delivery vehicles to allow additional deliveries of items in scarce supply including non-prescription medications such as anti-inflammatories, hygiene products such as toilet tissue, and basic food items. These are further problems resulting from lack of control of SARS-CoV2 infection/transmission.

WO2018/215766 describes a vaccine for MERS (Middle Eastern Respiratory Syndrome) coronavirus (MERS-CoV). One vector mentioned in this document is ChAdOx1. The vaccine comprises the full length MERS CoV spike protein with a human tPA leader added at the 5′ end. In one embodiment the relevant part of the nucleotide sequence is codon optimised for human use. However, when the inventors proceeded to manufacture in preparation for GMP (Good Manufacturing Practice) production, they found they could only do so using a tet repressed cell line, which is a problem. The inventors had numerous difficulties in manufacturing this vaccine at the desired scale and sought to overcome problem(s) associated with the prior art.

### SUMMARY OF THE INVENTION

In some embodiments, the present disclosure relates to a combination which comprises a simian adenoviral vector (such as ChAdOx1) delivering a SARS-CoV2 antigen (the spike protein). This combination has been produced with special attention to the nucleotide sequences encoding the antigen and in particular addressing technical problems of genetic stability and sequence rearrangements/mutations. This approach has delivered surprising technical benefits including efficient high yield production without the need for Tet repression, as well as intact virus being successfully rescued with correct cargo sequences preserved. A key benefit delivered by this new combination is the induction of strong immune responses after only a single vaccine administration. In some embodiments, the present disclosure relates to a composition comprising a viral vector, the viral vector comprising nucleic acid having a polynucleotide sequence encoding the spike protein from the coronavirus SARS-CoV2, wherein the viral vector is an adenovirus based vector.

In some embodiments, the adenovirus based vector is ChAdOx 1. In some embodiments, the spike protein comprises receptor binding domains (RBDs). In some embodiments, the spike protein is full length spike protein. In some embodiments, the spike protein is present as a fusion with the tissue plasminogen activator (tPA) sequence in the order N-terminus - tPA - spike protein - C-terminus. In some embodiments, the tPA comprises an amino acid sequence selected from the group consisting of SEQ ID NO: 5, SEQ ID NO: 6, SEQ ID NO: 7, and SEQ ID NO: 8. In some embodiments, the spike protein comprises the amino acid sequence SEQ ID NO: 1. In some embodiments, the polynucleotide sequence comprises an amino acid sequence selected from the group consisting of SEQ ID NO: 3 and SEQ ID NO: 4. In some embodiments, the viral vector sequence comprises the sequence of ECACC accession number 12052403. In some embodiments, the composition is configured such that administration of a single dose of the composition to a mammalian subject induces protective immunity in the subject. In some embodiments, the composition is configured such that administration of a first dose of the composition to a mammalian subject followed by administration of a second dose of the composition to the mammalian subject induces protective immunity in the subject. In some embodiments, the composition is configured for use in induction of an immune response against SARS-CoV2 in a mammalian subject. In some embodiments, the composition is configured for use in preventing SARS-CoV2 infection in a mammalian subject. In some embodiments, the present disclosure relates to a medicament for prevention of SARS-CoV2 infection in a mammalian subject.

In some embodiments, the present disclosure relates to a method of inducing an immune response against SARS-CoV2 in a mammalian subject, the method comprising administering a dose of the composition disclosed herein. In some embodiments, the method further comprises the steps of: administering a first dose of the composition to a subject; and administering a second dose of the composition to the subject, wherein the first dose and the second dose each comprise about the same number of viral particles. In some embodiments, the first and second dose each comprise about 5 x 1010 viral particles. In some embodiments, the second dose is administered at an interval of less than 6 weeks, 6 to 8 weeks, 9 to 11 weeks, or at least 12 weeks after administration of the first dose. In some embodiments, the composition is administered by a route of administration selected from a group consisting of intranasal, aerosol, intradermal and intramuscular.

In addition, the inventors describe the optional incorporation of a leader sequence/secretory sequence such as the tissue plasminogen activator (tPA) amino acid sequence fused to the N-terminus of the SARS-CoV2 spike protein antigen. This triple combination (ChAdOx1 + tPA + SARS-CoV2 spike protein) delivers enhanced immunogenicity. The inventors provide data demonstrating that a single dose of this combined construct delivers significant increases in the relevant immune responses - data demonstrating these advantages are provided in the Examples section below.

Thus, in one aspect the disclosure relates to a composition comprising a viral vector, the viral vector comprising nucleic acid having a polynucleotide sequence encoding the spike protein from the coronavirus SARS-CoV2, characterised in that said viral vector is an adenovirus based vector.

Suitably said adenovirus based vector is a simian adenovirus based vector. Suitably said adenovirus based vector is ChAdOx 1. Suitably said spike protein comprises the receptor binding domains (RBDs). Suitably said spike protein is full length spike protein.

Suitably said spike protein is present as a fusion with the tissue plasminogen activator (tPA) sequence in the order N-terminus - tPA - spike protein - C-terminus. Suitably said tPA has the amino acid sequence SEQ ID NO: 5, SEQ ID NO: 6, SEQ ID NO: 7, or SEQ ID NO: 8. Suitably said spike protein has the amino acid sequence SEQ ID NO: 1. Suitably said polynucleotide sequence comprises the sequence of SEQ ID NO: 3 or SEQ ID NO: 4, preferably SEQ ID NO: 4. Suitably said viral vector sequence is as in ECACC accession number 12052403.

Suitably administration of a single dose of a composition as described above to a mammalian subject induces protective immunity in said subject. Suitably administration of two doses of a composition as described above to a mammalian subject induces protective immunity in said subject. Suitably administration of a first dose of a composition as described above to a mammalian subject, followed by subsequent administration of a second dose of said composition to said subject, induces protective immunity in said subject.

In another embodiment the invention relates to use of a composition as described above for induction of, or for use in induction of, an immune response against SARS-CoV2. Suitably said immune response is an immune response in a mammalian subject.

In another embodiment the invention relates to a composition as described above for induction of, or for use in induction of, an immune response against SARS-CoV2 in a mammalian subject, wherein a single dose of said composition is administered to said subject.

In another embodiment the invention relates to a composition as described above for induction of, or for use in induction of, an immune response against SARS-CoV2 in a mammalian subject, wherein two doses of said composition are administered to said subject.

In another embodiment the invention relates to a composition as described above for induction of, or for use in induction of, an immune response against SARS-CoV2 in a mammalian subject, wherein a first dose of said composition is administered to said subject, and subsequently a second dose of said composition is administered to said subject.

In another embodiment the invention relates to a composition as described above for induction of, or for use in induction of, an immune response against SARS-CoV2 in a mammalian subject, wherein said composition is administered once. In another embodiment the invention relates to a composition as described above for induction of, or for use in induction of, an immune response against SARS-CoV2 in a mammalian subject, wherein said composition is administered twice.

In another embodiment the invention relates to a composition as described above for induction of, or for use in induction of, an immune response against SARS-CoV2 in a mammalian subject, wherein a first dose of said composition is administered to said subject, and subsequently a second dose of said composition is administered to said subject. Suitably said composition is administered once per 12 months. Suitably said composition is administered once per 60 months.

In another embodiment the invention relates to a composition as described above for preventing, or for use in preventing, SARS-CoV2 infection. Suitably preventing SARS-CoV2 infection is preventing SARS-CoV2 infection in a mammalian subject. In another embodiment the invention relates to a composition as described above for preventing, or for use in preventing, SARS-CoV2 infection in a mammalian subject, wherein a single dose of said composition is administered.

In another embodiment the invention relates to a composition as described above for preventing, or for use in preventing, SARS-CoV2 infection in a mammalian subject, wherein two doses of said composition are administered to said subject.

In another embodiment the invention relates to a composition as described above for preventing, or for use in preventing, SARS-CoV2 infection in a mammalian subject, wherein a first dose of said composition is administered to said subject, and subsequently a second dose of said composition is administered to said subject.

In another embodiment the invention relates to a composition as described above for preventing, or for use in preventing, SARS-CoV2 infection in a mammalian subject, wherein said composition is administered once. In another embodiment the invention relates to a composition as described above for preventing, or for use in preventing, SARS-CoV2 infection in a mammalian subject, wherein said composition is administered twice.

In another embodiment the invention relates to a composition as described above for preventing, or for use in preventing, SARS-CoV2 infection in a mammalian subject, wherein a first dose of said composition is administered to said subject, and subsequently a second dose of said composition is administered to said subject. Suitably said composition is administered once per 12 months. Suitably said composition is administered once per 60 months.

In another embodiment the invention relates to use of a composition as described above in medicine. In another embodiment the invention relates to a composition as described above for use in medicine. In another embodiment the invention relates to a composition as described above for use as a medicament. In another embodiment the invention relates to use of a composition as described above in the preparation of a medicament for prevention of, or for use in prevention of, SARS-CoV2 infection. Suitably prevention of SARS-CoV2 infection is prevention of SARS-CoV2 infection in a mammalian subject.

In another embodiment the invention relates to a method of inducing an immune response against SARS-CoV2 in a mammalian subject, the method comprising administering a composition as described above to said subject. In another embodiment the invention relates to a method of inducing an immune response against SARS-CoV2 in a mammalian subject, the method comprising administering a dose of a composition as described above to said subject.

In another embodiment the invention relates to a method as described above wherein a single dose of said composition is administered to said subject. Suitably said composition is administered once.

In another embodiment the invention relates to a method as described above wherein two doses of said composition are administered to said subject.

In another embodiment the invention relates to a method as described above wherein a first dose of said composition is administered to said subject, and subsequently a second dose of said composition is administered to said subject.

Suitably said composition is administered twice. Suitably said composition is administered once per 12 months. Suitably said composition is administered once per 60 months.

Suitably said composition is administered by a route of administration selected from a group consisting of intranasal, aerosol, intradermal and intramuscular. Suitably said administration is intranasal or intramuscular. Suitably said administration is intramuscular.

Suitably said spike protein is full length spike protein.

One example of a CoV spike protein sequence useful in the invention is vCoV-19 spike protein from Severe acute respiratory syndrome coronavirus 2 isolate Wuhan-Hu-1 i.e. the spike protein encoded by the viral genome with GenBank accession number MN908947.

More suitably said spike protein has the amino acid sequence as in (or as encoded in) the SARS-CoV2 genome of GenBank accession number MG772933.1 (Bat SARS-like coronavirus isolate bat-SL-CoVZC45). More suitably the SARS-CoV2 may be isolate bat-SL-CoVZC45. Most suitably said spike protein has the amino acid sequence of SEQ ID NO: 1. SEQ ID NO: 1- Amino acid sequence of SARS-CoV2 Spike protein only (no tPA fusion)

FVFLVLLPLVSSQCVNLTTRTQLPPAYTNSFTRGVYYPDKVFRSSVLHST
QDLFLPFFSNVTWFHAIHVSGTNGTKRFDNPVLPFNDGVYFASTEKSNI
I RGWIFGTTLDSKTQSLLIVNNATNVVIKVCEFQFCNDPFLGVYYHKNN
KS WMESEFRVYSSANNCTFEYVSQPFLMDLEGKQGNFKNLREFVFKNID
GYF KIYSKHTPINLVRDLPQGFSALEPLVDLPIGINITRFQTLLALHRS
YLTP GDSSSGWTAGAAAYYVGYLQPRTFLLKYNENGTITDAVDCALDPL
SETKC TLKSFTVEKGIYQTSNFRVQPTESIVRFPNITNLCPFGEVFNAT
RFASVY AWNRKRISNCVADYSVLYNSASFSTFKCYGVSPTKLNDLCFTN
VYADSFV IRGDEVRQIAPGQTGKIADYNYKLPDDFTGCVIAWNSNNLDS
KVGGNYNY LYRLFRKSNLKPFERDISTEIYQAGSTPCNGVEGFNCYFPL
QSYGFQPTN GVGYQPYRVVVLSFELLHAPATVCGPKKSTNLVKNKCVNF
NFNGLTGTGV LTESNKKFLPFQQFGRDIADTTDAVRDPQTLEILDITPC
SFGGVSVITPG TNTSNQVAVLYQDVNCTEVPVAIHADQLTPTWRVYSTG
SNVFQTRAGCLI GAEHVNNSYECDIPIGAGICASYQTQTNSPRRARSVA
SQSIIAYTMSLGAENSVAYSNNSIAIPTNFTISVTTEILPV SMTKTSVD
CTMYICGDSTECSNLLLQYGSFCTQLNRALTGIAVEQDKNTQ EVFAQVK
QIYKTPPIKDFGGFNFSQILPDPSKPSKRSFIEDLLFNKVTLA DAGFIK
QYGDCLGDIAARDLICAQKFNGLTVLPPLLTDEMIAQYTSALLA GTITS
GWTFGAGAALQIPFAMQMAYRFNGIGVTQNVLYENQKLIANQFNS AIGK
IQDSLSSTASALGKLQDVVNQNAQALNTLVKQLSSNFGAISSVLND ILS
RLDKVEAEVQIDRLITGRLQSLQTYVTQQLIRAAEIRASANLAATKM SE
CVLGQSKRVDFCGKGYHLMSFPQSAPHGVVFLHVTYVPAQEKNFTTAP A
ICHDGKAHFPREGVFVSNGTHWFVTQRNFYEPQIITTDNTFVSGNCDVV
IGIVNNTVYDPLQPELDSFKEELDKYFKNHTSPDVDLGDISGINASVVNI
QKEIDRLNEVAKNLNESLIDLQELGKYEQYIKWPWYIWLGFIAGLIAIV
M VTIMLCCMTSCCSCLKGCCSCGSCCKFDEDDSEPVLKGVKLHYT

SEQ ID NO: 11: Nucleotide sequence for spike protein from nCoV 19 genome (From GenBank Accession number MG772933.1)

ATGTTTGTTTTTCTTGTTTTATTGCCACTAGTCTCTAGTCAGTGTGTTAA
TCTTACAACCAGAACTCAATTACCCCCTGCATACACTAATTCTTTCACA
C GTGGTGTTTATTACCCTGACAAAGTTTTCAGATCCTCAGTTTTACATT
CA ACTCAGGACTTGTTCTTACCTTTCTTTTCCAATGTTACTTGGTTCCA
TGC TATACATGTCTCTGGGACCAATGGTACTAAGAGGTTTGATAACCCT
GTCC TACCATTTAATGATGGTGTTTATTTTGCTTCCACTGAGAAGTCTA
ACATA ATAAGAGGCTGGATTTTTGGTACTACTTTAGATTCGAAGACCCA
GTCCCT ACTTATTGTTAATAACGCTACTAATGTTGTTATTAAAGTCTGT
GAATTTC AATTTTGTAATGATCCATTTTTGGGTGTTTATTACCACAAAA
ACAACAAA AGTTGGATGGAAAGTGAGTTCAGAGTTTATTCTAGTGCGAA
TAATTGCAC TTTTGAATATGTCTCTCAGCCTTTTCTTATGGACCTTGAA
GGAAAACAGGGTAATTTCAAAAATCTTAGGGAATTTGTGTT TAAGAATA
TTGATGGTTATTTTAAAATATATTCTAAGCACACGCCTATTA ATTTAGT
GCGTGATCTCCCTCAGGGTTTTTCGGCTTTAGAACCATTGGTA GATTTG
CCAATAGGTATTAACATCACTAGGTTTCAAACTTTACTTGCTTT ACATA
GAAGTTATTTGACTCCTGGTGATTCTTCTTCAGGTTGGACAGCTG GTGC
TGCAGCTTATTATGTGGGTTATCTTCAACCTAGGACTTTTCTATTA AAA
TATAATGAAAATGGAACCATTACAGATGCTGTAGACTGTGCACTTGA CC
CTCTCTCAGAAACAAAGTGTACGTTGAAATCCTTCACTGTAGAAAAAG G
AATCTATCAAACTTCTAACTTTAGAGTCCAACCAACAGAATCTATTGTT
AGATTTCCTAATATTACAAACTTGTGCCCTTTTGGTGAAGTTTTTAACGC
CACCAGATTTGCATCTGTTTATGCTTGGAACAGGAAGAGAATCAGCAAC
T GTGTTGCTGATTATTCTGTCCTATATAATTCCGCATCATTTTCCACTT
TT AAGTGTTATGGAGTGTCTCCTACTAAATTAAATGATCTCTGCTTTAC
TAA TGTCTATGCAGATTCATTTGTAATTAGAGGTGATGAAGTCAGACAA
ATCG CTCCAGGGCAAACTGGAAAGATTGCTGATTATAATTATAAATTAC
CAGAT GATTTTACAGGCTGCGTTATAGCTTGGAATTCTAACAATCTTGA
TTCTAA GGTTGGTGGTAATTATAATTACCTGTATAGATTGTTTAGGAAG
TCTAATC TCAAACCTTTTGAGAGAGATATTTCAACTGAAATCTATCAGG
CCGGTAGC ACACCTTGTAATGGTGTTGAAGGTTTTAATTGTTACTTTCC
TTTACAATC ATATGGTTTCCAACCCACTAATGGTGTTGGTTACCAACCA
TACAGAGTAG TAGTACTTTCTTTTGAACTTCTACATGCACCAGCAACTG
TTTGTGGACCT AAAAAGTCTACTAATTTGGTTAAAAACAAATGTGTCAA
TTTCAACTTCAA TGGTTTAACAGGCACAGGTGTTCTTACTGAGTCTAAC
AAAAAGTTTCTGC CTTTCCAACAATTTGGCAGAGACATTGCTGACACTA
CTGATGCTGTCCGT GATCCACAGACACTTGAGATTCTTGACATTACACC
ATGTTCTTTTGGTGG TGTCAGTGTTATAACACCAGGAACAAATACTTCT
AACCAGGTTGCTGTTC TTTATCAGGATGTTAACTGCACAGAAGTCCCTG
TTGCTATTCATGCAGAT CAACTTACTCCTACTTGGCGTGTTTATTCTAC
AGGTTCTAATGTTTTTCA AACACGTGCAGGCTGTTTAATAGGGGCTGAA
CATGTCAACAACTCATATG AGTGTGACATACCCATTGGTGCAGGTATAT
GCGCTAGTTATCAGACTCAG ACTAATTCTCCTCGGCGGGCACGTAGTGT
AGCTAGTCAATCCATCATTGC CTACACTATGTCACTTGGTGCAGAAAAT
TCAGTTGCTTACTCTAATAACT CTATTGCCATACCCACAAATTTTACTA
TTAGTGTTACCACAGAAATTCTA CCAGTGTCTATGACCAAGACATCAGT
AGATTGTACAATGTACATTTGTGG TGATTCAACTGAATGCAGCAATCTT
TTGTTGCAATATGGCAGTTTTTGTA CACAATTAAACCGTGCTTTAACTG
GAATAGCTGTTGAACAAGACAAAAAC ACCCAAGAAGTTTTTGCACAAGT
CAAACAAATTTACAAAACACCACCAAT TAAAGATTTTGGTGGTTTTAAT
TTTTCACAAATATTACCAGATCCATCAA AACCAAGCAAGAGGTCATTTA
TTGAAGATCTACTTTTCAACAAAGTGACA CTTGCAGATGCTGGCTTCAT
CAAACAATATGGTGATTGCCTTGGTGATAT TGCTGCTAGAGACCTCATT
TGTGCACAAAAGTTTAACGGCCTTACTGTTTTGCCACCTTTGCTCACAGA
TGAAATGATTGCTCAATACAC TTCTGCACTGTTAGCGGGTACAATCACT
TCTGGTTGGACCTTTGGTGCAG GTGCTGCATTACAAATACCATTTGCTA
TGCAAATGGCTTATAGGTTTAAT GGTATTGGAGTTACACAGAATGTTCT
CTATGAGAACCAAAAATTGATTGC CAACCAATTTAATAGTGCTATTGGC
AAAATTCAAGACTCACTTTCTTCCA CAGCAAGTGCACTTGGAAAACTTC
AAGATGTGGTCAACCAAAATGCACAA GCTTTAAACACGCTTGTTAAACA
ACTTAGCTCCAATTTTGGTGCAATTTC AAGTGTTTTAAATGATATCCTT
TCACGTCTTGACAAAGTTGAGGCTGAAG TGCAAATTGATAGGTTGATCA
CAGGCAGACTTCAAAGTTTGCAGACATAT GTGACTCAACAATTAATTAG
AGCTGCAGAAATCAGAGCTTCTGCTAATCT TGCTGCTACTAAAATGTCA
GAGTGTGTACTTGGACAATCAAAAAGAGTTG ATTTTTGTGGAAAGGGCT
ATCATCTTATGTCCTTCCCTCAGTCAGCACCT CATGGTGTAGTCTTCTT
GCATGTGACTTATGTCCCTGCACAAGAAAAGAA CTTCACAACTGCTCCT
GCCATTTGTCATGATGGAAAAGCACACTTTCCTC GTGAAGGTGTCTTTG
TTTCAAATGGCACACACTGGTTTGTAACACAAAGG AATTTTTATGAACC
ACAAATCATTACTACAGACAACACATTTGTGTCTGG TAACTGTGATGTT
GTAATAGGAATTGTCAACAACACAGTTTATGATCCTT TGCAACCTGAAT
TAGACTCATTCAAGGAGGAGTTAGATAAATATTTTAAG AATCATACATC
ACCAGATGTTGATTTAGGTGACATCTCTGGCATTAATGC TTCAGTTGTA
AACATTCAAAAAGAAATTGACCGCCTCAATGAGGTTGCCA AGAATTTAA
ATGAATCTCTCATCGATCTCCAAGAACTTGGAAAGTATGAG CAGTATAT
AAAATGGCCATGGTACATTTGGCTAGGTTTTATAGCTGGCTT GATTGCC
ATAGTAATGGTGACAATTATGCTTTGCTGTATGACCAGTTGCT GTAGTT
GTCTCAAGGGCTGTTGTTCTTGTGGATCCTGCTGCAAATTTGAT GAAGA
CGACTCTGAGCCAGTGCTCAAAGGAGTCAAATTACATTACACATA A

Suitably the nucleic acid encoding the spike protein antigen, and/or encoding the tPA-spike protein antigen fusion, is codon optimised for humans.

Suitably the nucleic acid encoding the spike protein antigen, and/or encoding the tPA-spike protein antigen fusion, is substituted to eliminate runs of repeat nucleotides such as 5 or more consecutive occurrences of the same nucleotide.

Suitably the nucleic acid encoding the spike protein antigen, and/or encoding the tPA-spike protein antigen fusion, is codon optimised for humans and is substituted to eliminate runs of repeat nucleotides such as 5 or more consecutive occurrences of the same nucleotide.

Suitably said polynucleotide sequence comprises the sequence of SEQ ID NO: 3.

This presents a nucleotide sequence as revised by the inventor (i.e. after codon optimisation for humans introduced runs of same bases and after those runs of same bases were revised to retain the same coding sequence but remove the repeats) without tPA encoded.

SEQ ID NO: 3 - Without tPA leader

TTCGTGTTCCTGGTGCTGCTGCCACTGGTGTCTAGCCAGTGTGTGAACCT
GACCACCAGGACACAGCTGCCTCCAGCCTACACCAACAGCTTCACCAGA
G GCGTGTACTACCCAGACAAGGTGTTCAGATCCAGCGTGCTGCACTCTA
CC CAGGACCTGTTCCTGCCTTTCTTCAGCAACGTGACCTGGTTCCACGC
CAT CCACGTGTCCGGCACCAATGGCACCAAGAGATTCGACAACCCAGTG
CTGC CCTTCAACGACGGAGTGTACTTTGCCAGCACCGAGAAGTCCAACA
TCATC AGAGGCTGGATCTTCGGCACCACACTGGACAGCAAGACCCAGAG
CCTGCT GATCGTGAACAACGCCACCAACGTGGTCATCAAAGTGTGCGAG
TTCCAGT TCTGCAACGACCCATTCCTGGGCGTCTACTACCACAAGAACA
ACAAGAGC TGGATGGAAAGCGAGTTCCGGGTGTACAGCAGCGCCAACAA
CTGCACCTT CGAGTACGTGTCCCAGCCTTTCCTGATGGACCTGGAAGGC
AAGCAGGGCA ACTTCAAGAACCTGCGCGAGTTCGTGTTCAAGAACATCG
ACGGCTACTTC AAGATCTACAGCAAGCACACACCTATCAACCTCGTGCG
GGATCTGCCTCA GGGCTTCTCTGCTCTGGAACCACTGGTGGATCTGCCC
ATCGGCATCAACA TCACCCGGTTTCAGACACTGCTGGCCCTGCACAGAA
GCTACCTGACACCT GGCGATAGCAGCTCTGGATGGACAGCTGGCGCCGC
TGCCTACTATGTGGG ATACCTGCAGCCTCGGACCTTCCTGCTGAAGTAC
AACGAGAACGGCACCA TCACCGACGCCGTGGATTGTGCTCTGGATCCTC
TGAGCGAGACAAAGTGC ACCCTGAAGTCCTTCACCGTGGAAAAGGGCAT
CTACCAGACCAGCAACTT CCGGGTGCAGCCCACCGAATCCATCGTGCGG
TTCCCGAATATCACCAATC TGTGCCCATTCGGCGAGGTGTTCAATGCCA
CCAGATTCGCCTCTGTGTAC GCCTGGAACCGGAAGCGGATCAGCAATTG
CGTGGCCGACTACTCCGTGCT GTACAACTCCGCCAGCTTCAGCACCTTC
AAGTGCTACGGCGTGTCACCTA CCAAGCTGAACGACCTGTGCTTCACAA
ACGTGTACGCCGACAGCTTCGTG ATCCGTGGAGATGAAGTGCGGCAGAT
TGCACCTGGACAGACAGGCAAGAT CGCCGACTACAACTACAAGCTGCCC
GACGACTTCACCGGCTGTGTGATTG CCTGGAACAGCAACAACCTGGATA
GCAAAGTCGGCGGCAACTACAATTAC CTGTACCGGCTGTTCCGGAAGTC
CAATCTGAAGCCCTTCGAGCGGGACAT CTCCACCGAGATCTATCAGGCC
GGCAGCACACCTTGTAACGGCGTGGAAG GCTTCAACTGCTACTTCCCAC
TGCAGTCCTACGGCTTTCAGCCCACAAAT GGCGTGGGCTACCAGCCTTA
CAGAGTGGTGGTGCTGAGCTTCGAGCTGCT GCATGCTCCTGCCACAGTG
TGCGGCCCTAAGAAAAGCACCAATCTCGTGA AGAACAAATGCGTGAACT
TCAACTTCAACGGCCTGACCGGCACCGGCGTG CTGACAGAGAGCAACAA
GAAGTTCCTGCCATTCCAGCAGTTCGGCCGGGA TATCGCCGATACCACA
GATGCCGTCAGAGATCCTCAGACACTGGAAATCC TGGACATCACACCTT
GCAGCTTCGGCGGAGTGTCTGTGATCACGCCTGGC ACCAACACCAGCAA
TCAGGTGGCAGTGCTGTACCAGGACGTGAACTGTAC CGAAGTGCCCGTG
GCCATTCACGCCGATCAGCTGACACCTACATGGCGGG TGTACTCCACCG
GCAGCAATGTGTTTCAGACCAGAGCCGGCTGTCTGATC GGAGCCGAGCA
CGTGAACAATAGCTACGAGTGCGACATCCCTATCGGCGC TGGCATCTGC
GCCTCTTACCAGACACAGACAAACAGCCCTAGACGGGCCAGATCTGTGGC
CAGCCAGAGCATCA TTGCCTACACAATGTCTCTGGGCGCCGAGAACAGC
GTGGCCTACTCCAAC AACTCTATCGCTATCCCGACCAACTTCACCATCA
GCGTGACCACAGAGAT CCTGCCTGTGTCCATGACCAAGACCAGCGTGGA
CTGCACCATGTACATCT GCGGCGATTCCACCGAGTGCTCCAACCTGCTG
CTGCAGTACGGCAGCTTC TGCACCCAGCTGAATAGAGCCCTGACAGGGA
TCGCCGTGGAACAGGACAA GAACACCCAAGAGGTGTTCGCCCAAGTGAA
GCAGATCTACAAGACGCCTC CTATCAAGGACTTCGGCGGCTTCAATTTC
AGCCAGATTCTGCCCGATCCT AGCAAGCCCAGCAAGCGGAGCTTCATCG
AGGACCTGCTGTTCAACAAAGT GACACTGGCCGACGCCGGCTTCATCAA
GCAGTATGGCGATTGTCTGGGCG ACATTGCCGCCAGGGATCTGATTTGC
GCCCAGAAGTTTAACGGACTGACA GTGCTGCCTCCTCTGCTGACCGATG
AGATGATCGCCCAGTACACATCTGC CCTGCTGGCCGGCACAATCACAAG
CGGCTGGACATTTGGAGCTGGCGCTG CCCTGCAGATCCCATTTGCTATG
CAGATGGCCTACCGGTTCAACGGCATC GGAGTGACCCAGAATGTGCTGT
ACGAGAACCAGAAGCTGATCGCCAACCA GTTCAACAGCGCCATCGGCAA
GATCCAGGACAGCCTGAGCAGCACAGCAA GCGCCCTGGGAAAGCTGCAG
GACGTGGTCAACCAGAATGCCCAGGCACTG AACACCCTGGTCAAGCAGC
TGTCTAGCAACTTCGGCGCCATCAGCTCTGT GCTGAACGATATCCTGAG
CAGACTGGACAAGGTGGAAGCCGAGGTGCAGA TCGACAGACTGATCACC
GGAAGGCTGCAGTCCCTGCAGACCTACGTTACC CAGCAGCTGATCAGAG
CCGCCGAGATTAGAGCCTCTGCCAATCTGGCCGC CACCAAGATGTCTGA
GTGTGTGCTGGGCCAGAGCAAGAGAGTGGACTTTT GCGGCAAGGGCTAC
CACCTGATGAGCTTCCCTCAGTCTGCGCCTCACGGC GTGGTGTTTCTGC
ACGTGACATACGTGCCCGCTCAAGAGAAGAATTTCAC CACCGCTCCAGC
CATCTGCCACGACGGCAAAGCCCACTTTCCTAGAGAAG GCGTGTTCGTG
TCCAACGGCACCCATTGGTTCGTGACCCAGCGGAACTTC TACGAGCCTC
AGATCATCACCACCGACAACACCTTCGTGTCTGGCAACTG CGACGTCGT
GATCGGCATTGTGAACAATACCGTGTACGACCCTCTGCAGC CCGAGCTG
GACAGCTTCAAAGAGGAACTGGATAAGTACTTTAAGAACCAC ACAAGCC
CTGACGTGGACCTGGGCGATATCAGCGGAATCAATGCCAGCGT CGTGAA
CATCCAGAAAGAGATCGACCGGCTGAACGAGGTGGCCAAGAATC TGAAC
GAGAGCCTGATCGACCTGCAAGAACTGGGCAAGTACGAGCAGTAC ATCA
AGTGGCCCTGGTACATCTGGCTGGGCTTTATCGCCGGACTGATTGC CAT
CGTGATGGTCACAATCATGCTGTGTTGCATGACCAGCTGCTGTAGCT GC
CTGAAGGGCTGTTGTAGCTGTGGCTCCTGCTGCAAGTTCGACGAGGAC G
ATTCTGAGCCCGTGCTGAAGGGCGTGAAACTGCACTACACCTGA

Most suitably said polynucleotide sequence comprises the sequence of SEQ ID NO: 4 This presents the preferred nucleotide sequence as revised by the inventor (i.e. after codon optimisation for humans introduced runs of same bases and after those runs of same bases were revised to retain the same coding sequence but remove the repeats) with tPA encoded. This is a highly preferred embodiment of the invention.

SEQ ID NO: 4 - With section encoding tPA leader underlined

ATGGACGCCATGAAGCGAGGCCTGTGCTGTGTTCTGCTTCTGTGTGGCGC CGTGTTTGTGTCCGCCAGCCAAGAGATCCACGCCAGATTTCGGAGATTCG
TGTTCCTGGTGCTGCTGCCACTGGTGTCTAGCCAGTGTGTGAACCT GAC
CACCAGGACACAGCTGCCTCCAGCCTACACCAACAGCTTCACCAGAG GC
GTGTACTACCCAGACAAGGTGTTCAGATCCAGCGTGCTGCACTCTACC C
AGGACCTGTTCCTGCCTTTCTTCAGCAACGTGACCTGGTTCCACGCCAT
CCACGTGTCCGGCACCAATGGCACCAAGAGATTCGACAACCCAGTGCTGC
CCTTCAACGACGGAGTGTACTTTGCCAGCACCGAGAAGTCCAACATCAT
C AGAGGCTGGATCTTCGGCACCACACTGGACAGCAAGACCCAGAGCCTG
CTGATCGTGAACAAC GCCACCAACGTGGTCATCAAAGTGTGCGAGTTCC
AGTTCTGCAACGACCC ATTCCTGGGCGTCTACTACCACAAGAACAACAA
GAGCTGGATGGAAAGCG AGTTCCGGGTGTACAGCAGCGCCAACAACTGC
ACCTTCGAGTACGTGTCC CAGCCTTTCCTGATGGACCTGGAAGGCAAGC
AGGGCAACTTCAAGAACCT GCGCGAGTTCGTGTTCAAGAACATCGACGG
CTACTTCAAGATCTACAGCA AGCACACACCTATCAACCTCGTGCGGGAT
CTGCCTCAGGGCTTCTCTGCT CTGGAACCACTGGTGGATCTGCCCATCG
GCATCAACATCACCCGGTTTCA GACACTGCTGGCCCTGCACAGAAGCTA
CCTGACACCTGGCGATAGCAGCT CTGGATGGACAGCTGGCGCCGCTGCC
TACTATGTGGGATACCTGCAGCCT CGGACCTTCCTGCTGAAGTACAACG
AGAACGGCACCATCACCGACGCCGT GGATTGTGCTCTGGATCCTCTGAG
CGAGACAAAGTGCACCCTGAAGTCCT TCACCGTGGAAAAGGGCATCTAC
CAGACCAGCAACTTCCGGGTGCAGCCC ACCGAATCCATCGTGCGGTTCC
CGAATATCACCAATCTGTGCCCATTCGG CGAGGTGTTCAATGCCACCAG
ATTCGCCTCTGTGTACGCCTGGAACCGGA AGCGGATCAGCAATTGCGTG
GCCGACTACTCCGTGCTGTACAACTCCGCC AGCTTCAGCACCTTCAAGT
GCTACGGCGTGTCACCTACCAAGCTGAACGACCTGTGCTTCACAAACG T
GTACGCCGACAGCTTCGTGATCCGTGGAGATGAAGTGCGGCAGATTGCA
CCTGGACAGACAGGCAAGATCGCCGACTACAACTACAAGCTGCCCGACGA
CTTCACCGGCTGTGTGATTGCCTGGAACAGCAACAACCTGGATAGCAAA
G TCGGCGGCAACTACAATTACCTGTACCGGCTGTTCCGGAAGTCCAATC
TG AAGCCCTTCGAGCGGGACATCTCCACCGAGATCTATCAGGCCGGCAG
CAC ACCTTGTAACGGCGTGGAAGGCTTCAACTGCTACTTCCCACTGCAG
TCCT ACGGCTTTCAGCCCACAAATGGCGTGGGCTACCAGCCTTACAGAG
TGGTG GTGCTGAGCTTCGAGCTGCTGCATGCTCCTGCCACAGTGTGCGG
CCCTAA GAAAAGCACCAATCTCGTGAAGAACAAATGCGTGAACTTCAAC
TTCAACG GCCTGACCGGCACCGGCGTGCTGACAGAGAGCAACAAGAAGT
TCCTGCCA TTCCAGCAGTTCGGCCGGGATATCGCCGATACCACAGATGC
CGTCAGAGA TCCTCAGACACTGGAAATCCTGGACATCACACCTTGCAGC
TTCGGCGGAG TGTCTGTGATCACGCCTGGCACCAACACCAGCAATCAGG
TGGCAGTGCTGTACCAGGACGTGAACTGTACCGAAGTGCCC GTGGCCAT
TCACGCCGATCAGCTGACACCTACATGGCGGGTGTACTCCAC CGGCAGC
AATGTGTTTCAGACCAGAGCCGGCTGTCTGATCGGAGCCGAGC ACGTGA
ACAATAGCTACGAGTGCGACATCCCTATCGGCGCTGGCATCTGC GCCTC
TTACCAGACACAGACAAACAGCCCTAGACGGGCCAGATCTGTGGC CAGC
CAGAGCATCATTGCCTACACAATGTCTCTGGGCGCCGAGAACAGCG TGG
CCTACTCCAACAACTCTATCGCTATCCCGACCAACTTCACCATCAGC GT
GACCACAGAGATCCTGCCTGTGTCCATGACCAAGACCAGCGTGGACTG C
ACCATGTACATCTGCGGCGATTCCACCGAGTGCTCCAACCTGCTGCTGC
AGTACGGCAGCTTCTGCACCCAGCTGAATAGAGCCCTGACAGGGATCGCC
GTGGAACAGGACAAGAACACCCAAGAGGTGTTCGCCCAAGTGAAGCAGA
T CTACAAGACGCCTCCTATCAAGGACTTCGGCGGCTTCAATTTCAGCCA
GA TTCTGCCCGATCCTAGCAAGCCCAGCAAGCGGAGCTTCATCGAGGAC
CTG CTGTTCAACAAAGTGACACTGGCCGACGCCGGCTTCATCAAGCAGT
ATGG CGATTGTCTGGGCGACATTGCCGCCAGGGATCTGATTTGCGCCCA
GAAGT TTAACGGACTGACAGTGCTGCCTCCTCTGCTGACCGATGAGATG
ATCGCC CAGTACACATCTGCCCTGCTGGCCGGCACAATCACAAGCGGCT
GGACATT TGGAGCTGGCGCTGCCCTGCAGATCCCATTTGCTATGCAGAT
GGCCTACC GGTTCAACGGCATCGGAGTGACCCAGAATGTGCTGTACGAG
AACCAGAAG CTGATCGCCAACCAGTTCAACAGCGCCATCGGCAAGATCC
AGGACAGCCT GAGCAGCACAGCAAGCGCCCTGGGAAAGCTGCAGGACGT
GGTCAACCAGA ATGCCCAGGCACTGAACACCCTGGTCAAGCAGCTGTCT
AGCAACTTCGGC GCCATCAGCTCTGTGCTGAACGATATCCTGAGCAGAC
TGGACAAGGTGGA AGCCGAGGTGCAGATCGACAGACTGATCACCGGAAG
GCTGCAGTCCCTGC AGACCTACGTTACCCAGCAGCTGATCAGAGCCGCC
GAGATTAGAGCCTCT GCCAATCTGGCCGCCACCAAGATGTCTGAGTGTG
TGCTGGGCCAGAGCAA GAGAGTGGACTTTTGCGGCAAGGGCTACCACCT
GATGAGCTTCCCTCAGT CTGCGCCTCACGGCGTGGTGTTTCTGCACGTG
ACATACGTGCCCGCTCAA GAGAAGAATTTCACCACCGCTCCAGCCATCT
GCCACGACGGCAAAGCCCA CTTTCCTAGAGAAGGCGTGTTCGTGTCCAA
CGGCACCCATTGGTTCGTGA CCCAGCGGAACTTCTACGAGCCTCAGATC
ATCACCACCGACAACACCTTC GTGTCTGGCAACTGCGACGTCGTGATCG
GCATTGTGAACAATACCGTGTA CGACCCTCTGCAGCCCGAGCTGGACAG
CTTCAAAGAGGAACTGGATAAGT ACTTTAAGAACCACACAAGCCCTGAC
GTGGACCTGGGCGATATCAGCGGA ATCAATGCCAGCGTCGTGAACATCC
AGAAAGAGATCGACCGGCTGAACGA GGTGGCCAAGAATCTGAACGAGAG
CCTGATCGACCTGCAAGAACTGGGCA AGTACGAGCAGTACATCAAGTGG
CCCTGGTACATCTGGCTGGGCTTTATC GCCGGACTGATTGCCATCGTGA
TGGTCACAATCATGCTGTGTTGCATGAC CAGCTGCTGTAGCTGCCTGAA
GGGCTGTTGTAGCTGTGGCTCCTGCTGCA AGTTCGACGAGGACGATTCT
GAGCCCGTGCTGAAGGGCGTGAAACTGCAC TACACCTGA

Suitably said spike protein has the amino acid sequence of SEQ ID NO: 10 - Amino acid sequence of tPA-Spike fusion (tPA underlined)

MDAMKRGLCCVLLLCGAVFVSASQEIHARFRRFVFLVLLPLVSSQCVNLT
TRTQLPPAYTNSFTRGVYYPDKVFRSSVLHST QDLFLPFFSNVTWFHAI
HVSGTNGTKRFDNPVLPFNDGVYFASTEKSNII RGWIFGTTLDSKTQSL
LIVNNATNVVIKVCEFQFCNDPFLGVYYHKNNKS WMESEFRVYSSANNC
TFEYVSQPFLMDLEGKQGNFKNLREFVFKNIDGYF KIYSKHTPINLVRD
LPQGFSALEPLVDLPIGINITRFQTLLALHRSYLTP GDSSSGWTAGAAA
YYVGYLQPRTFLLKYNENGTITDAVDCALDPLSETKC TLKSFTVEKGIY
QTSNFRVQPTESIVRFPNITNLCPFGEVFNATRFASVY AWNRKRISNCV
ADYSVLYNSASFSTFKCYGVSPTKLNDLCFTNVYADSFV IRGDEVRQIA
PGQTGKIADYNYKLPDDFTGCVIAWNSNNLDSKVGGNYNY LYRLFRKSN
LKPFERDISTEIYQAGSTPCNGVEGFNCYFPLQSYGFQPTN GVGYQPYR
VVVLSFELLHAPATVCGPKKSTNLVKNKCVNFNFNGLTGTGV LTESNKK
FLPFQQFGRDIADTTDAVRDPQTLEILDITPCSFGGVSVITPG TNTSNQ
VAVLYQDVNCTEVPVAIHADQLTPTWRVYSTGSNVFQTRAGCLI GAEHV
NNSYECDIPIGAGICASYQTQTNSPRRARSVASQSIIAYTMSLGA ENSV
AYSNNSIAIPTNFTISVTTEILPVSMTKTSVDCTMYICGDSTECSN LLL
QYGSFCTQLNRALTGIAVEQDKNTQEVFAQVKQIYKTPPIKDFGGFN FS
QILPDPSKPSKRSFIEDLLFNKVTLADAGFIKQYGDCLGDIAARDLIC A
QKFNGLTVLPPLLTDEMIAQYTSALLAGTITSGWTFGAGAALQIPFAMQ
MAYRFNGIGVTQNVLYENQKLIANQFNSAIGKIQDSLSSTASALGKLQDV
VNQNAQALNTLVKQLSSNFGAISSVLNDILSRLDKVEAEVQIDRLITGR
L QSLQTYVTQQLIRAAEIRASANLAATKMSECVLGQSKRVDFCGKGYHL
MS FPQSAPHGVVFLHVTYVPAQEKNFTTAPAICHDGKAHFPREGVFVSN
GTH WFVTQRNFYEPQIITTDNTFVSGNCDVVIGIVNNTVYDPLQPELDS
FKEE LDKYFKNHTSPDVDLGDISGINASVVNIQKEIDRLNEVAKNLNES
LIDLQ ELGKYEQYIKWPWYIWLGFIAGLIAIVMVTIMLCCMTSCCSCLK
GCCSCG SCCKFDEDDSEPVLKGVKLHYT

Suitably the primary vaccination regimen is one dose. In some embodiments it may be desired to re-administer at a later date. Intervals between first and second doses are disclosed in the examples. In some embodiments it may be desired to re-administer at a later date, not less than 6 months after the first immunisation. Suitably it may be desired to re-administer at a later date, such as about 12 months after the first immunisation. Suitably it may be desired to re-administer at a later date, such as about 12 to 60 months after the first immunisation. In one embodiment suitably a second or further administration is given at about 12 months after the first immunisation. In one embodiment suitably a second or further administration is given at about 60 months after the first immunisation.

In one embodiment suitably a second or further administration is given more than 60 months after the first immunisation. In one embodiment suitably an even later second or further administration is even better.

In one aspect, the invention relates to use of a composition as described above in medicine. In one aspect, the invention relates to use of a composition as described above in the preparation of a medicament for prevention of SARS-CoV2 infection.

In another aspect, the invention relates to use of a composition as described above in inducing an immune response against SARS-CoV2. In another aspect, the invention relates to use of a composition as described above in immunising a subject against SARS-CoV2. In another aspect, the invention relates to use of a composition as described above in prevention of SARS-CoV2 infection. A method of inducing an immune response against SARS-CoV2 in a mammalian subject, the method comprising administering a composition as described above to said subject. Suitably a single dose of said composition is administered to said subject.

Suitably said composition is administered once. Suitably said composition may be administered once per 6 months. More suitably said composition is administered once per 12 months. More suitably said composition is administered once per 60 months.

Suitably said composition is administered by a route of administration selected from a group consisting of subcutaneous, intranasal, aerosol, nebuliser, intradermal and intramuscular. Most suitably said administration is intramuscular or intranasal. Most suitably said administration is intramuscular.

In one aspect the invention relates to an adeno-based viral vector comprising nucleic acid having a polynucleotide sequence encoding the spike protein from SARS-CoV2. Suitably the adeno-based viral vector is ChAdOx 1.

In one aspect, the invention relates to a ChAdOx vector comprising a polynucleotide encoding glycoprotein S from the SARS-CoV2 virus. Suitably said adeno-based viral vector has the sequence and/or construction as described in one or more of the examples.

In one aspect, the invention relates to a method of raising an immune response by administering the adeno-based viral vector as described above.

In one aspect, the invention relates to the adeno-based viral vector as described above for use in preventing SARS-CoV2 infection.

In one aspect, the invention relates to the adeno-based viral vector as described above for use in raising an anti- SARS-CoV2 immune response.

### BRIEF DESCRIPTION OF THE DRAWINGS

The present invention will now be described by way of example, with reference to the accompanying drawings, in which:

FIG. 1 is a histogram that shows total IU within an 80 ml culture infected at MOI 3 with and without repression.

FIG. 2 is a histogram that shows a decrease in total IU in a dose dependent manner according to MOI.

FIG. 3 is a histogram that shows genome copy number within flasks depicted as percentage standardising 100% as output from de-repressed culture.

FIGS. 4A and 4B are histograms that show summed splenic IFN-γ ELISpot responses of BALB/c and CD-1 mice, respectively, in response to peptides spanning the spike protein from SARS-CoV-2, nine or ten days post vaccination, with 1.7 × 10¹⁰ vp ChAdOx1 nCoV-19 or 8 × 10⁹ vp ChAdOx1 GFP.

FIGS. 5A and 5B depict box and whisker plot of the optical densities following ELISA analysis of BALB/C mouse sera incubated with purified protein spanning the S1 domain or purified protein spanning the S2 domain, respectively, of the SARS-CoV-2 spike nine or ten days post vaccination, with 1.7 × 10¹⁰ vp ChAdOx1 nCoV-19 or 8 × 10⁹ vp ChAdOx1 GFP. FIGS. 5C and 5D depict box and whisker plots of the optical densities following ELISA analysis of CD-1 mouse sera incubated with purified protein spanning the S1 domain or purified protein spanning the S2 domain, respectively, of the SARS-CoV-2 spike.

FIG. 6 is a chart that shows a DNA map of ChAdOx1 nCoV-19.

FIGS. 7A and 7B are histograms that show end point titer (EPT) of serum IgG detected against S1 or S2 protein, respectively. in BALB/c (circles) or CD1 (squares) mice. FIG. 7C shows summed Spike-specific IFNγ ELISpot responses measured in BALB/c (circles) and outbred CD1 (squares) mice.

FIGS. 7D and 7E are histograms that show the summed frequency of Spike-specific cytokine positive CD4 or CD8 T cells, respectively, as measured by intracellular cytokine staining following stimulation of splenocytes peptides in BALB/c (circles) and CD1(squares) mice.

FIGS. 8A, 8B, 8C and 8D are histograms that show IgG subclass antibodies detected against S1 protein in BALB/c mice, S1 protein in CD1 mice, S2 protein in BALB/c mice, and S2 protein in CD1 mice, respectively. FIGS. 8E and 8F show IFNγ ELISpot responses following stimulation of splenocytes with S1 pool (black) or S2 pool (grey) in BALB/c (circle) and outbred CD1(square) mice, respectively. FIGS. 8G, 8H, 8I and 8J are histograms that show the frequency of cytokine positive CD4 T cells in BALB/c mice, cytokine positive CD4 T cells in CD1 mice, cytokine positive CD8 T cells in BALB/c mice and cytokine positive CD8 T cells in CD1 mice, respectively, as measured by intracellular cytokine staining following stimulation of splenocytes with S1 pool (black) or S2 pool (grey) peptides. FIGS. 8K and 8L are histograms that show fold change in cytokine levels in supernatant from S1 (black) and S2 (grey) stimulated splenocytes when compared to unstimulated splenocytes for BALB/c and CD1 mice, respectively.

FIGS. 9A, 9B, 9C and 9D are plots that show clinical observation of weight following SARS-CoV2 virus challenge in Rhesus macaques vaccinated with ChAdOx1 nCoV-19 (Group 1) or phosphate buffered saline (Group 2) and challenged 4 weeks later with 5.0 × 10⁶ pfu SARS-CoV2 virus with animals weights measured on each day post-challenge (DPC) and plotted as absolute figures (A and B) and percentage weight change (C and D).

FIGS. 10A, 10B, 10C and 10D are plots that show clinical observation of temperature following SARS-CoV2 virus challenge in Rhesus macaques vaccinated with ChAdOx1 nCoV-19 (Group 1) or phosphate buffered saline (Group 2) and challenged 4 weeks later with 5.0 × 10⁶ pfu SARS-CoV2 virus with temperatures measured pre-challenge (A and B) and post-challenge (C and D).

FIGS. 11A and 11B are plots that show COVID-19 total disease score and COVID-19 disease distribution score, respectively, from CT images measured using a quantitative score system developed for human COVID-19 in Rhesus macaques 5 days following immunization i.m. with 2.5 × 10¹⁰ viral particles of ChAdOx1 nCoV-19 or phosphate buffered saline (no vaccine) and challenged 4 weeks later with 5.0 × 10⁶ pfu SARS-CoV2 virus.

FIG. 12 is a histogram that shows nAb levels in 6 macaques at week 4 post vaccination.

FIGS. 13A-13J show SARS-CoV-2 S-specific T cell responses following ChAdOx1 nCoV-19 prime-only and prime-boost vaccination regimens in mice and pigs. FIG. 13A is a histogram that shows SARS-CoV-2 S protein-specific murine splenocyte responses by IFN-γ ELISpot assay in prime-only and prime-boost vaccination regimens, in BALB/c and CD1 mice. FIGS. 13B, 13C, 13D and 13E are histograms that show intracellular cytokine staining (ICS) of CD8+ T cells from BALB/c mice, CD8+ T cells from CD1 mice, CD4+ T cells from BALB/c mice, and CD4+ T cells from CD1 mice, respectively. FIG. 13F shows IFN-γ ELISpot analysis of porcine peripheral blood mononuclear cells (PBMC) in prime-boost and prime-only pigs. FIGS. 13G, 13H, 13I and 13J are histograms that show ICS for CD8+ T cells at day 14 post-vaccination, CD8+ T cells at day 42 post-vaccination, CD4+ T cells at day 14 post-vaccination, and CD4+ T cells at day 42 post-vaccination, respectively.

FIGS. 14A, 14B and 14C are plots that show antibody units or end-point titres (EPT) assessed by ELISA using recombinant SARS-CoV-2 FL-S for mice and pigs, and recombinant S protein RBD for pigs, respectively. FIGS. 14D and 14E show SARS-CoV-2 neutralising antibody titres in pig sera determined by VNT, expressed as the reciprocal of the serum dilution that neutralised virus infectivity in 50% of the wells, and pVNT, expressed as reciprocal serum dilution to inhibit pseudovirus entry by 50%, respectively.

FIGS. 15A, 15B, 15C, and 15D are histograms that show local adverse reactions in first 7 days post-vaccination with prophylactic ChAdOx1 nCoV-19 with paracetamol, ChAdOx1 nCoV-19 without paracetamol, MenACWY with paracetamol, MenACWY without paracetamol, respectively. FIGS. 15E, 15F, 15G, and 15H are histograms that show systemic adverse reactions in first 7 days post-vaccination with prophylactic ChAdOx1 nCoV-19 with paracetamol, ChAdOx1 nCoV-19 without paracetamol, MenACWY with paracetamol, MenACWY without paracetamol, respectively. P= 60 minute post-vaccination observation period in the clinic; Day 0 is the day of vaccination. Fever: Self-reported feeling feverishness, Fevertemp: objective fever measurements, mild: >= 38° C., moderate: >=38.5° C., severe: >=39.0° C.

FIGS. 16A and 16B are histograms that show local adverse reactions in first 7 days post-vaccination with prophylactic ChAdOx1 nCoV-19 first dose and ChAdOx1 nCoV-19 second dose, respectively.

FIGS. 16C and 16D are histograms that show systemic adverse reactions in first 7 days post-vaccination with prophylactic ChAdOx1 nCoV-19 first dose and ChAdOx1 nCoV-19 second dose, respectively, in a non-randomised subset of 10 participants. P= 60 minute post-vaccination observation period in the clinic; Day 0 is the day of vaccination. Fever: Self-reported feeling feverishness, Fevertemp: objective fever measurements, mild: >= 38° C., moderate: >=38.5° C., severe: >=39.0° C.

FIG. 17 shows SARS-CoV-2 IgG response by standardised in-house ELISA to spike protein in MenACWY recipients, ChAdOx1 nCoV-19 recipients, and convalescent sera from PCR+ COVID-19 patients. Error bars show median and IQR. Participants in boost group received second dose at day 28.

FIGS. 18A and 18B are plots showing multiplex SARS-CoV-2 IgG response by ELISA to spike protein and receptor binding domain, respectively, ChAdOx1 nCoV-19 recipients (red), MenACWY recipients (blue), and convalescent sera (green) from PCR+ COVID-19 patients. Error bars show median and IQR. Day 42 samples taken in N=9 participants boosted at day 28.

FIG. 19 shows IFNγ ELISpot response to peptides spanning the SARS-CoV-2 spike protein in ChAdOx1 nCoV-19 recipients and MenACWY recipients. SFC: Spot-forming cells, PBMC: Peripheral blood mononuclear cells, Error bars show medians and inter-quartile ranges. LLD is 48 SFC.

FIG. 20 is a plot showing a pseudotype neutralisation assay in MenACWY recipients (blue), ChAdOx1 nCoV-19 recipients (red), and convalescent sera (green) from COVID-19 cases (CONV). Solid lines connect samples from the same participant. Day 35 and Day 42 samples are from participants who received a booster dose at day 28.

FIGS. 21A, 21B and 21C are plots that show live SARS-CoV-2 virus neutralisation (IC100 - Marburg assay), live SARS-CoV-2 micro-neutralisation (MNA) (IC50 - Public Health England) and plaque reduction neutralisation titre (PRNT) assay (IC50 - Public Health England), respectively, in MenACWY recipients and ChAdOx1 nCoV-19 recipients. Group 1: Prime-only group, Group 3: Prime-boost group (boosted at day 28). Solid lines connect samples from the same participant. Dotted line shows lower/upper limits of detection. CONV: convalescent sera from COVID-19 cases, HCW+: Sera from health care workers who tested positive at baseline by ELISA.

FIG. 22 is a diagram that shows the trial profile.

FIGS. 23A and 23B are diagrams that show the effect of prophylactic paracetamol on solicited local reactions in the first 2 days after vaccination with ChAdOx1 nCoV-19 or MenACWY, respectively. Odds ratios were adjusted for age, sex, occupation (Health care worker or not), smoking, alcohol consumption and BMI.

FIGS. 24A and 24B are diagrams that show the effect of prophylactic paracetamol on solicited systemic reactions in the first 2 days after vaccination with ChAdOx1 nCoV-19 or MenACWY, respectively. Odds ratios were adjusted for age, sex, occupation (Health care worker or not), smoking, alcohol consumption and BMI.

FIGS. 25A and 25B are plots that show pseudotype neutralisation (IC50) in ChAdOx1 nCoV-19 recipients with standardised ELISA and with live virus neutralisation as measured by IC100 (Marburg), respectively.

FIGS. 26A, 26B, and 26C are histograms relating to Experiment 16 that show local adverse reactions in first 7 days after priming with standard dose vaccine by age for groups 5d, 7, and 8. FIGS. 26D, 26E, and 26F are histograms relating to Experiment 16that show local adverse reactions in first 7 days after boosting with standard dose vaccine by age for groups 5d, 7, and 8.

FIGS. 27A, 27B, and 27C are histograms relating to Experiment 16 that show systemic adverse reactions in first 7 days after priming with standard dose vaccine by age for groups 5d, 7, and 8. FIGS. 27D, 27E, and 27F are histograms relating to Experiment 16 that show systemic adverse reactions in first 7 days after boosting with standard dose vaccine by age for groups 5d, 7, and 8. Feverish: Self-reported feeling of feverishness, Fever: objective fever measurements, mild: >= 38oC, moderate: >=38.5oC, severe: >=39.0oC. Participants shown are those randomised to receive 2 doses.

FIGS. 28A, 28B, and 28C are graphs relating to Experiment 16 that show neutralising antibody titres measured in pseudotyped virus neutralisation assay after prime and boost vaccination in low dose ChadOx1 nCoV-19 recipients and MenACWY recipients aged 18-55, 56-69 and 70+, respectively.

FIGS. 28D, 28E and 28F are graphs relating to Experiment 16 that show neutralising antibody titres measured in pseudotyped virus neutralisation assay after prime and boost vaccination in high dose ChadOx1 nCoV-19 recipients and MenACWY recipients aged 18-55, 56-69 and 70+, respectively. Dotted line is lower limit of assay (40). Only participants allocated to receive two doses are shown. Comparison of day 42 titres across age groups receiving same dose, by ANOVA applied to log₂-transformed values: Low-dose, p= 0.2440, High-dose, p=0.6555. Comparison of day 42 titres across dose groups in each age cohort, by independent samples t-test applied to log₂-transformed values: 18-55, p= 0.5115, 56-69, p= 0.4516, 70+, p= 0.7664.

FIG. 29 shows a graph relating to Experiment 16 of Interferon-γ ELISpot response to peptides spanning the SARS-CoV-2 spike insert after prime and booster vaccination by age group and vaccine dose relating to MenACWY recipients and ChAdOx1 nCoV-19 recipients. Solid lines connect samples from the same participant. SFC: Spot-forming cells, PBMC: Peripheral blood mononuclear cells, boxes show medians and inter-quartile ranges. LLD is 48 SFC/M (dotted line). Day 42 samples are from participants who received a booster dose at day 28.

FIGS. 30A and 30B are graphs relating to Experiment 16 that show SARS-CoV-2 IgG response to the receptor binding domain and spike protein, respectively, by age and vaccine dose measured using a multiplex immunoassay (MIA) in high dose vaccine groups. FIGS. 30C and 30D are graphs that show SARS-CoV-2 IgG response to the receptor binding domain and spike protein, respectively, by age and vaccine dose measured using a multiplex immunoassay (MIA) in low dose vaccine groups.RBD: receptor binding domain; Spike: SARS-COV-2 spike protein. Participants in boost group received their second dose at day 28 (dotted line). Plot shows median and interquartile range. Control groups not shown.

FIGS. 31A and 31B are graphs relating to Experiment 16 that show neutralising antibody titres measured using a live virus SARS-CoV-2 microneutralisation assay (PHE - MNA80) after prime and boost vaccination by age and vaccine dose in high dose vaccine groups and low dose vaccine groups, respectively. Participants in boost group received their second dose at day 28. Plot shows median and interquartile range. Control groups not shown. To normalise data across assay runs, a reference sample was included in all assay runs and test samples normalised to this value by generating log10 ratios. Dotted lines show upper and lower limits of assay (values outside this range set to 640 and 5 respectively).

FIGS. 32A and 32B are graphs that show multiplex SARS-CoV-2 IgG response to the receptor binding domain and spike protein, respectively, by multiplex immunoassay after Prime-Boost.

FIG. 33 shows graphs of Live SARS-CoV-2 microneutralisation after Prime-Boost in relation to Example 17.

FIGS. 34A, 34B and 34C are graphs that show SARS-CoV-2 spike-specific immunoglobulin isotype responses induced by prime-boost regimens of ChAdOx1 nCoV-19 at two standard doses (SD) given 56 days apart, one standard dose and one low dose (LD) given 56 days apart, and two standard doses given 28 days apart, respectively, in relation to Example 17.

FIGS. 35A, 35B and 35C are graphs that show SARS-CoV-2 spike-specific IgG1 and IgG3 responses induced by prime-boost regimens of ChAdOx1 nCoV-19 at two standard doses (SD) given 56 days apart, one standard dose and one low dose (LD) given 56 days apart, and two standard doses given 28 days apart, respectively, in relation to Example 17. FIGS. 35D, 35E and 35F are graphs that show SARS-CoV-2 spike-specific IgG2 and IgG4 responses induced by prime-boost regimens of ChAdOx1 nCoV-19 at two standard doses (SD) given 56 days apart, one standard dose and one low dose (LD) given 56 days apart, and two standard doses given 28 days apart, respectively, in relation to Example 17.

FIG. 36 shows graphs of IFNγ ELISpot response to peptides spanning the SARS-CoV-2 spike vaccine insert after vaccination with ChAdOx1 nCoV-19 in relation to Example 17.

FIGS. 37A, 37B and 37C are graphs that show neutralising antibody measured in pseudovirus assay (Monogram IC50) at two standard doses (SD) given 28 days apart, two standard doses given 56 days apart, and one standard dose and one low dose (LD) given 56 days apart, respectively, in relation to Example 17.

FIGS. 38A, 38B, 38C, 38D and 38E are images that show the total lymphocyte population and activation of lymphocyte populations at 0, 7, 14 and 28 days post ChAdOx1 nCoV-19 vaccination, respectively, in relation to Example 18. FIG. 38F is a chart that shows expression levels of Ki67 in IgG+ B cells and NK cells and CD69 expression levels in CD4+ T cells and CD8+ T cells at 0, 7, 14 and 28 days post ChAdOx1 nCoV-19 vaccination. FIG. 38G is a chart that shows expression levels of Ki67 in B cells, CD4+ T cells and CD8+ T cells at 0, 7, 14 and 28 days post ChAdOx1 nCoV-19 vaccination. FIG. 38H is a chart that shows TNF-α and IFN-γ expression levels in CD4+ T cells at 0, 7, 14 and 28 days post ChAdOx1 nCoV-19 vaccination. FIG. 38I is a plot that shows cytokine expression levels at 7 days post-vaccination following antigen specific stimulation of PBMC.

FIGS. 39A, 39B and 39C are plots that show IgG, IgM and IgA responses, respectively, induced by ChAdOx1 nCoV-19 or MenACWY vaccination in relation to Example 18. FIG. 39D is a plot that shows total IgG avidity at various time points post-vaccination. FIG. 39E is a plot that shows change from baseline between IgG avidity values on day 28 and day 56 in the prime-only and prime-boost groups.

FIGS. 40A, 40B, 40C and 40D are plots that show IgG1, IgG3, IgG2, and IgG4 responses, respectively, induced by a single dose or prime-boost regimen of ChAdOx1 nCoV-19 in relation to Example 18. FIGS. 40E and 40F are a chart and a histogram, respectively, that show a correlation between IgG3 levels and neutralisation capacity for the same vaccines, demonstrating a correlation between IgG3 levels and MNA₈₀.

FIG. 41A is a plot that shows IFNγ ELISPOT responses to pools of 15 mer peptides covering the ChAdOx1-nCOV19 vaccine in relation to Example 18. FIG. 41B is a chart that shows responses to the individual pools at D14 from FIG. 41A plotted as fold-change from D0.

FIG. 42 is a plot that shows fold-change in SFC to each peptide pool for every ChAdOx1 vaccinated participant from baseline (D0) to D14 postvaccination in relation to Example 18.

FIG. 43A is a plot that shows antigen-specific cytokine secretion from CD4⁺ T cells and CD8⁺ T cells 14 days after a single dose of ChAdOx1 nCoV-19. FIG. 43B is a plot that shows CD8⁺ T cells expression of the degranulation marker CD107a. FIG. 43C is a bar and whisker plot that shows cytokine expression level in CD4⁺ T cells, and FIG. 43D shows the ratio of Th1 to Th2 expression levels from FIG. 43C. FIG. 43E is a plot that shows the frequency of cytokine positive cells at day 14 from participants with positive pre-vaccination T cell and antibody responses to SARS-CoV-2.

FIGS. 43F and 43G are pie charts that show that few multifunctional T cells were detected in either the CD4⁺ or CD8⁺ populations, respectively.

FIGS. 44A, 44B, 44C, 44D, and 44E show Cryo-ET and subtomogram average of ChAdOx1 nCoV-19 derived spike. FIG. 44A is an image showing a 6.4 Å thick tomographic slice of U2OS cell transduced with ChAdOx1 nCoV-19; PM = plasma membrane, scale bar = 100 nm. FIG. 44B is an image of a detailed view of the boxed area marked in FIG. 44A. White arrowheads indicate spike proteins on the cell surface; scale bar = 50 nm. FIGS. 44C, 44D and 44E are images of a 9.6 Å subtomogram average of ChAdOx1 nCoV-19 derived spike shown from side view, top view, and transversal section, respectively. SARS-CoV-2 S atomic model (PDB ID: 6VXX) was fitted for reference.

FIGS. 45A, 45B and 45C show site-specific glycan processing of SARS-CoV-2 S upon infection with ChAdOx1 nCoV-19. FIG. 45A is an image of a Western blot analysis of SARS-CoV-2 spike proteins, using anti-S1 and anti-S1+S2 antibodies. Lane 1= Protein pellet from 293F cell lysates infected with ChAdOx1 nCoV-19. Lane 2= Reduced protein pellet from 293F infected with ChAdOx1 nCoV-19. Lane 3=2P-stablilsed SARS-CoV-2 S protein. The white boxes correspond to gel bands that were excised for mass spectrometric analysis. FIG. 45B is a histogram that shows site-specific N-linked glycosylation of SARS-CoV-2 S0 and S1/S2 glycoproteins. LC-MS analysis. The bar graphs represent the relative quantities of digested glycopeptides possessing the identifiers of oligomannose/hybrid-type glycans, complex-type glycans, and unoccupied PNGs at each N-linked glycan sequon on the S protein, listed from N to C terminus. FIG. 45C is a figure that shows a glycosylated model of the cleaved (S1/S2) SARS-CoV-2 spike with pie charts summarizing the quantitative mass spectrometric analysis of the oligomannose/hybrid, complex, or unoccupied N-linked glycan populations. Representative glycans are modelled onto the prefusion structure of trimeric SARS-CoV-2 S glycoprotein (PDB ID: 6VSB), with one RBD in the “up” conformation.

FIG. 46A is a drawing that shows a cartoon of an immunization strategy that enhances the CD8 T cell response to ChAdOx1 nCoV-19 in aged mice. FIGS. 46B, 46C, 46D and 46D are histograms that show the percentage of Ki67⁺, CXCR3⁺, effector memory CD44⁺CD62L⁻ and central memory CD44⁺CD62L⁺ CD8⁺ T cells, respectively, in the draining aortic lymph node from 3-month-old (3 mo) or 22-month-old (22 mo) mice nine days after immunization with ChAdOx1 nCoV-19 or PBS. FIG. 46F is a histogram that shows the percentage of proliferating Ki67⁺ splenic CD8⁺ T cells in 3-month-old (3 mo) or 22-month-old (22 mo) mice nine days after immunization with ChAdOx1 nCoV-19 or PBS. FIG. 46G is a graph that shows the number of CD8⁺ cells producing granzyme B (GZMB), IFNγ, IL-2 or TNFα six hours after restimulation with SARS-CoV-2 peptide pools, in FIG. 46H the number of single and double cytokine producing CD8 T cells are represented in stacked bar charts. Spleen cells are taken from 3-month-old (3 mo) or 22-month-old (22 mo) mice nine days after immunization with ChAdOx1 nCoV-19 or PBS. FIG. 46I depicts a cartoon of a prime-boost immunization strategy. FIGS. 46J, 46K, 46L and 46M are histograms that show the percentage of Ki67⁺, CXCR3⁺, effector memory CD44⁺CD62L⁻ and central memory CD44⁺CD62L⁺ CD8⁺ T cells, respectively, in the draining aortic lymph node from 3-month-old or 22-month-old mice nine days after immunization with ChAdOx1 nCoV-19 or PBS. FIGS. 46O and 46P are histograms that show the percentage of proliferating granzyme B-producing and IFNγ-producing splenic CD8+ T cells, respectively, in 3-month-old or 22-month-old mice nine days after immunization with ChAdOx1 nCoV-19 or PBS. FIG. 46P is a histogram that shows the number of single and double cytokine producing CD8 T cells as represented in stacked bar charts. Spleen cells are taken from 3-month-old or 22-month-old mice nine days after immunization with ChAdOx1 nCoV-19 or PBS. Bar height in FIGS. 46B-G and 46J-O corresponds to the median and each circle represents one biological replicate. In FIGS. 46H and P, each bar segment represents the mean and the error bars the standard deviation. The Shapiro-Wilk normality test was used to determine whether the data are consistent with a normal distribution, followed by either an ordinary one-way ANOVA test for data with a normal distribution or a Kruskal Wallis test for non-normally distributed data alongside a multiple comparisons test. Data are representative of two independent experiments (n=4-8 per group/experiment).

FIG. 47A is a cartoon that shows the prime immunization strategy. FIGS. 47B, 47C and 47D are histograms that show the percentage of proliferating Ki67⁺, CXCR3⁺CD44⁺ CD4 T cells, and CXCR3⁺CD44⁺Foxp3⁺ Treg cells, respectively, in the draining aortic lymph node. FIGS. 47E, 47F and 47G are histograms that show the percentage of proliferating Ki67⁺, CXCR3⁺CD44⁺ CD4 T cells and CXCR3⁺CD44⁺Foxp3⁺ Treg cells, respectively, in the spleen of 3-month-old or 22-month-old mice nine days after immunization with ChAdOx1 nCoV-19 or PBS. FIGS. 47H and 47I are charts that show the number of CD4⁺Foxp3⁻ cells producing IFNγ, IL-2, IL-4, IL-5, IL-17 or TNFα six hours after restimulation with SARS-CoV-2 peptide pools and the number of single and multiple cytokine producing CD4 T cells as represented in stacked bar charts, respectively. FIG. 47J is a cartoon that shows the prime-boost immunization strategy. FIGS. 47K, 47L, and 47M are histograms that show the percentage of proliferating Ki67⁺, CXCR3⁺CD44⁺ CD4 T cells, and CXCR3⁺CD44⁺Foxp3⁺ Treg cells, respectively, in the draining aortic lymph node. FIGS. 47N, 47O and 47P are histograms that show the percentage of Ki67⁺ CD44⁺, CXCR3⁺CD44⁺ CD4⁺Foxp3⁻ T cells, and CXCR3⁺CD44⁺Foxp3⁺ Treg cells, respectively, in the spleen of 3-month-old or 22-month-old mice nine days after immunization with ChAdOx1 nCoV-19 or PBS. FIGS. 47Q and 47R are graphs that show the number of CD4⁺Foxp3⁻ cells producing IFNγ, IL-2, IL-4, IL-5, IL-17 or TNFα six hours after restimulation with SARS-CoV-2 peptide pools and the number of single and multiple cytokine producing CD4 T cells as represented in stacked bar charts, respectively. Bar height in corresponds to the median and each circle represents one biological replicate. In FIGS. 47I and 47R, each bar segment represents the mean and the error bars the standard deviation. The Shapiro-Wilk normality test was used to determine whether the data are consistent with a normal distribution, followed by either an ordinary one-way ANOVA test for data with a normal distribution or a Kruskal Wallis test for non-normally distributed data alongside a multiple comparisons test. Data are representative of two independent experiments (n=4-8 per group/experiment).

FIGS. 48A-48U show impaired B cell responses after ChAdOx1 nCoV-19 immunisation of aged mice. B cell response in 3-month-old (3 mo) or 22-month-old (22 mo) mice nine days after immunization with ChAdOx1 nCoV-19 or PBS. FIGS. 48A and 48B are histograms that show the results of flow cytometric evaluation of the percentage and number, respectively, of plasma cells in the aortic lymph node. FIG. 48C shows two pie charts that show the proportion of IgM⁺IgD⁻ and switched IgM⁻IgD⁻ plasma cells from FIGS. 48A and 48B. FIGS. 48D and 48E are histograms that show serum IgM and IgG anti-spike antibodies, respectively, nine days after immunization. FIG. 48F shows two pie charts that show the proportion of anti-spike IgG of the indicated subclasses in the serum nine days after immunisation. FIGS. 48G and 48H are histograms that show the percentage and number, respectively, of germinal centre B cells in the aortic lymph node. FIG. 48I shows pie charts that show the proportion of IgM⁺IgD⁻ and switched IgM⁻IgD⁻ germinal centre cells from FIGS. 48G and 48H. FIGS. 48J and 48K are histograms that show the number of T follicular helper and T follicular regulatory cells, respectively, in the draining lymph node. FIGS. 48L and 48M are confocal images of the spleen of ChAdOx1 nCoV-19 immunised mice of the indicated ages with the scale bars representing 500 µm and 50 µm in FIGS. 48L and 48M, respectively. FIGS. 48N and 48O are plots that show the percentage and number, respectively, of splenic germinal centre B cells. FIG. 48P is a histogram that shows the percentage of Ki67⁺ B cells in the spleen. FIGS. 48Q and 48R are plots that show the number of splenic T follicular helper and T follicular regulatory cells, respectively. FIGS. 48S and 48T are histograms that show serum IgM and IgG anti-spike antibodies, respectively, and FIG. 48U is a chart that shows IgG subclasses 28 days after immunization. Bar height in corresponds to the median and each circle represents one biological replicate. The Shapiro-Wilk normality test was used to determine whether the data are consistent with a normal distribution, followed by either an ordinary one-way ANOVA test for data with a normal distribution or a Kruskal Wallis test for non-normally distributed data alongside a multiple comparisons test. Data are representative of two independent experiments (n=4-8 per group/experiment).

FIGS. 49A-49O show a booster immunization enhances the B cell response to ChAdOx1 nCoV-19 immunisation in aged mice. FIG. 49A is a diagram that shows the scheme of the prime-boost immunization protocol. FIG. 49B is a histogram that shows the percentage of Ki67⁺ B cells in the draining lymph node. FIGS. 49C and 49D are histograms that show the percentage and number, respectively, of plasma cells in the aortic lymph node. FIG. 49E shows pie charts showing the proportion of IgM⁺IgD⁻ and switched IgM⁻IgD⁻ plasma cells from FIGS. 49B and 49C. FIGS. 49F and 49G show the percentage and number, respectively, of germinal centre B cells in the aortic lymph node. FIG. 49H shows pie charts showing the proportion of IgM⁺IgD⁻ and switched IgM⁻IgD⁻ germinal centre cells from FIGS. 49F and 49G. FIGS. 49I and 49J are histograms that show the number of T follicular helper and T follicular regulatory cells, respectively, in the draining lymph node. FIG. 49K is a histogram that shows percentage of splenic germinal centre B cells. FIGS. 49L and 49M are plots that show serum anti-spike IgM and IgG subclasses, respectively. FIGS. 49N and 49O are plots that show serum anti-spike IgG subclasses prior to boost (day 29) and nine days after boost immunization, respectively. FIGS. 49P and 49Q are histograms that show SARS-CoV-2 neutralising antibody titres in sera at 9 days post-prime and 9 day post-boost, respectively, as determined by micro neutralisation test, expressed as reciprocal serum dilution to inhibit pseudovirus entry by 50% (IC₅₀). Samples below the lower limit of detection (LLoD) are shown as half of the LLoD. Bar height in corresponds to the median and each circle represents one biological replicate. The Shapiro-Wilk normality test was used to determine whether the data are consistent with a normal distribution, followed by either an ordinary one-way ANOVA test for data with a normal distribution or a Kruskal Wallis test for non-normally distributed data alongside a multiple comparisons test.

FIGS. 49B-49O are shown from one of two independent experiments (n=4-8 per group/experiment), whereas in FIGS. 49P and 49Q the data are pooled from two experiments.

FIGS. 50A, 50B, and 50C are histograms that show viral load in nasal wash, oral swabs, and rectal swabs, respectively, as measured by qRT-PCR on Day 3 post challenge.

FIGS. 51A, 51B, and 51C are histograms that show viral load in nasal wash, oral swabs, and rectal swabs, respectively, as measured by qRT-PCR on Day 5 post challenge.

FIGS. 52A, 52B, and 52C are histograms that show viral load in nasal wash, oral swabs, and rectal swabs, respectively, as measured by qRT-PCR on Day 7 post challenge.

FIGS. 53A, 53B, and 53C are histograms that show viral load in nasal wash, oral swabs, and rectal swabs, respectively, as measured by qRT-PCR on Day 9 post challenge.

FIGS. 54A and 54B are Kaplan-Meier plots that show cumulative incidence of symptomatic NAAT+ COVID-19 disease after 1 or more standard doses or after 2 doses, respectively.

### DETAILED DESCRIPTION

Coronavirus 19 (SARS-CoV2; nCoV-19; sometimes referred to as COVID-19) means the virus responsible for an outbreak of coronavirus disease in humans that was first reported from Wuhan, China, on 31 Dec. 2019. The virus is now properly known as SARS-CoV2. The disease it causes is COVID-19. More specifically SARS-CoV2 means the virus having a genome comprising the nucleotide sequence of accession number MN908947 or MG772933.1, most suitably MG772933.1. Suitably antibodies induced as described herein are neutralising antibodies i.e. antibodies capable of neutralising SARS-CoV2 viral particles.

The inventors have made a vaccine against SARS-CoV2. Preclinical data show excellent results - we refer to the examples section below.

Production of both research grade vaccine suitable for pre-clinical studies and a pre-GMP vaccine seed stock were initiated as soon as the SARS-CoV2 sequence was released. The vaccine design comprises the complete SARS-CoV2 Spike protein expressed under the control of a strong mammalian promoter, which includes Tet repressor sequences to allow for repression of antigen expression during vaccine manufacture, improving vaccine yields. Preparation of the vaccine for pre-clinical studies went well and mouse immunisation experiments were immediately undertaken (see examples section).

The inventors teach rapid manufacturing and clinical development of ChAdOx1 SARS-CoV2.

Suitably the composition of the invention comprises ChAdOx1 :: SARS-CoV2 spike protein i.e. ChAdOx1 comprising a nucleic acid insert having a nucleotide sequence encoding the SARS-CoV2 spike protein. Suitably the full length spike protein is used. Suitably a human tPA leader sequence is added at the 5′ end. Suitably the nucleotide sequence is codon optimised for human codon use.

In addition, the inventor further studied the sequence and devised the idea to remove runs of repeated bases from the sequence. In more detail, the inventor first codon optimised the coding sequence of the antigen for human codon usage. More suitably, the inventor codon optimised the nucleotide sequence encoding the tPA-SARS-CoV2 spike protein antigen fusion for human codon usage. However, as explained above, based on intellectual insights in reviewing the sequence the inventor noticed that there were several patches in which the human codon optimisation process had resulted in runs of identical nucleotides. For example, runs of 5 consecutive “C” bases (cytosine bases) were noticed. The inventor devised the idea that these repetitive sequences might be causing problems in expression, leading to problems of vaccine performance, and/or polymerase “slippage” events, leading to problems in viral vector vaccine production due to nucleic acid instability (e.g. mutations, rearrangements such as truncations etc). In order to address these technical problems, the inventor came up with the idea to further mutate the already mutated codon optimised sequence. Thus, the inventor proceeded to design and make further substitutions in the nucleotide sequence, carefully preserving the encoded amino acids using the universal genetic code, whilst changing the nucleotide bases and selecting alternate codons to remove the slippage prone repeat sequences whilst ensuring the coding sequence still accurately encoded the desired antigen. As can be seen from the data presented in the examples section, their approach was very successful and delivered the technical benefit of facilitating viral vector vaccine production, obtaining good yields of virus. The virus obtained in this manner also demonstrated excellent immunogenicity and other properties discussed herein.

The inventors were surprised that:
Vaccine yields of the SARS-CoV2 viral vector composition appear to be the same with and without tet repression. The inventors found this to be astonishing. In view of the problems and drawbacks encountered in preparing GMP manufacture of the MERS-CoV vaccine described in WO2018/215766 (discussed above in Background section) the view before this invention was that for all viral glycoproteins tet repression would be needed. The view was that these viral glycoproteins are toxic, hence the requirement for Tet repression during manufacture. The invention demonstrates the surprising benefit that Tet repression is NOT required for manufacture of the SARS-CoV2 viral vector composition.
It has been remarkably quick and easy to rescue virus from plasmid. Preparations of virus have been made in record time. Some of these preparations have even been made in record time despite including the now unnecessary tet repression steps in the procedure. Therefore the speed and ease of rescuing virus from plasmid is a further technical benefit delivered by the invention.
Immunogenicity in mice at 10 days is really strong. We refer to the examples section below.

In more detail, it should be noted that the inventors had been researching a revised version of the viral vector encoding the MERS spike protein (i.e. as described in WO2018/215766) with runs of repeated bases removed. After a huge amount of work by these experienced researchers who are at the forefront of this field of research, it still had not been possible to prepare a seed stock of viral vector encoding the MERS spike protein that was genetically correct. At best when the inventors found the MERS spike antigen was correct, there was then one or more spontaneous deletion(s) in the promoter region. These observations show firstly that removing runs of repeated bases does not fully explain the effect/drawbacks with the MERS composition of the prior art. These observations show secondly that removing runs of repeated bases is not a simple or straightforward approach that can simply be applied with an expectation of success, since if this were the case then the expectation would have been that the MERS composition with runs of repeated bases removed would have worked, which it did not. This is further evidence of unpredictability in the art and evidence of the surprising effects of the present invention.

In more detail, a further problem experienced by the inventors in different areas of their research had lead them to the conclusion that viral glycoproteins were consistently toxic in the viral particle production systems used for manufacture. The inventors had therefore concluded that Tet repression would always be necessary in order to avoid toxicity issues. This hypothesis was reinforced by their observations working with internal viral protein antigens, which did not appear to suffer from the same toxicity problems as viral glycoproteins. The present invention employing the SARS-CoV2 spike protein is a clear exception to this rule and is further evidence towards inventive step.

In more detail, even the choice of viral sequence to use to derive the antigenic sequences of the invention was an intellectual choice which had to be made. For example, the first sequence for SARS-CoV-2 was released on Friday the 10th of January 2020. Further viral sequences were released over the following days, for example:

Viral Sequences that were released:In more detail, even the choice of viral sequence to use to derive the antigenic sequences of the invention was an intellectual choice which had to be made. For example, the first sequence for SARS-CoV-2 was released on Friday the 10th of January 2020. Further viral sequences were released over the following days, for example:

Viral Sequences that were released

| Virus name | Accession ID | Passage details/history | Collection date | Host | Originating lab | Submitting lab |
|---|---|---|---|---|---|---|
| BetaCoV/Wuhan/IVDC-HB-01/2019 | EPI_ISL_402119 | Virus Isolate, Passage 1 | 2019-12-30 | Human | National Institute for Viral Disease Control and Prevention, China CDC | National Institute for Viral Disease Control and Prevention, China CDC |
| BetaCoV/Wuhan/IVDC-HB-04/2020 | EPI_ISL_402120 | Original | 2020-01-01 | Human | National Institute for Viral Disease Control and Prevention, China CDC | National Institute for Viral Disease Control and Prevention, China CDC |
| BetaCoV/Wuhan/IVDC-HB-05/2019 | EPI_ISL_402121 | Original | 2019-12-30 | Human | National Institute for Viral Disease Control and Prevention, China CDC | National Institute for Viral Disease Control and Prevention, China CDC |
| BetaCoV/Wuhan/IPBCAMS-WH-01/2019 | EPI_ISL_402123 | Original | 2019-12-24 | Human | Institute of Pathogen Biology, Chinese Academy of Medical Sciences & Peking Union Medical College | Institute of Pathogen Biology, Chinese Academy of Medical Sciences & Peking Union Medical College |
| BetaCoV/Wuhan/WIV04/2019 | EPI_ISL_402124 | Original | 2019-12-30 | Human | Wuhan Jinyintan Hospital | Wuhan Institute of Virology, Chinese Academy of Sciences |

It was not apparent which of the sequences would be best to include in the compositions/vaccine, for example due to suspected polymorphisms.

Analysis was carried out including a number of sequence alignments and the viral sequence to be included in the compositions of the invention was selected based on this analysis.

Mouse immunogenicity data is provided to demonstrate beneficial properties of the invention (see examples section).

It is a further advantage of the invention that really strong antibody and T-cell responses were obtained in mice after only 10 days.

It is believed that the combination of ChAdOx1 with the SARS-CoV2 antigen used in this work has not been disclosed previously and is therefore novel.

Prior art prime-boost using MVA based vaccine candidates produces a very robust immune response as has been demonstrated repeatedly with a large number of different antigens in various indications. It is an advantage of the invention that one administration of ChAdOx- SARS-CoV2 raises a robust immune response. This response from a single dose of ChAdOx- SARS-CoV2 was unexpectedly strong and has a number of benefits including quicker, simpler treatment and cheaper manufacturing and treatment.

We disclose that ChAdOx1 based vaccine compositions described herein against SARS-CoV2 elicit antibodies and cellular immune responses in mice.

We describe four vaccines against SARS-CoV2 based on ChAdOx1 and MVA viral vectors, two vaccines per vector. All vaccines contain the full-length spike gene of SARS-CoV2; ChAdOx1 SARS-CoV2 vaccines were produced with or without the leader sequence of the human tissue plasminogen activator gene (tPA) where MVA SARS-CoV2 vaccines were produced with tPA, and either the mH5 or F11 promoter driving expression of the spike gene.

We disclose development of vaccine candidates that are based on two different viral vectors: Chimpanzee Adenovirus, Oxford University #1 (ChAdOx1) (26) and Modified Vaccinia virus Ankara (MVA) (27, 28). Each viral vector was developed by generating two alternative versions, resulting in four vaccine candidates that all encode the same complete SARS-CoV2 spike gene (S). The two ChAdOx1 based vaccines were produced with or without the signal peptide of the human tissue plasminogen activator gene (tPA) at the N terminus. Previous studies have shown that encoding tPA upstream of recombinant antigens enhanced immunogencity, although results differed depending on the antigens employed. The tPA encoded upstream of influenza A virus nucleoprotein, in a DNA vector, enhanced both cellular and humoral immune responses in mice (29, 30), whereas the same leader sequence resulted in increased humoral sequences but decreased cellular responses to HIV Gag (30). The two MVA based vaccines were produced with either the mH5 or F11 poxviral promoter driving antigen expression, both including the tPA sequence at the N terminus of SARS-CoV2 Spike protein. Previously, we reported the ability of the strong early F11 promoter to enhance cellular immunogenicity of vaccine antigen candidates for malaria and influenza, as compared to utilising p7.5 or mH5 early/late promoters which resulted in a lower level of gene expression immediately after virus infection of target cells, but higher levels at a later stage (31). Here, we continue to assess the F11 promoter in enhancing cellular immunogenicity, and to investigate its ability to impact on humoral immune responses.

The inventors identified the major surface antigen of SARS-CoV2 as the Spike (S protein) and demonstrated that ChAdOx1 expressing this protein induces the production of anti-S antibodies, after a single intramuscular immunisation.

### Prime-Boost

The invention also finds application in prime-boost immunisation regimes. For example, if after a period of time the immune response declines, as naturally tends to happen for many immune responses, then it may be desired to boost the response in a patient back to useful levels such as protective levels.

Boosting may be homologous boosting i.e. may be attained a second administration of the same composition as used for the original priming immunisation. In another embodiment, the boosting immunisation may be carried out using a different composition to the composition used for the original priming immunisation. This is referred to as heterologous prime boost. Suitably the heterologous boost (i.e. the second for further immunisation) comprises one or more compositions selected from MVA, RNA, DNA, protein, adenovirus based viral vector, simian adenovirus based viral vector, gorilla-based adenovirus based viral vector, or human adenovirus based viral vector. More suitably the boosting (second or further) immunisation may comprise MVA, RNA or protein. Most suitably, the boost (second or further immunisation) may comprise RNA or protein.

Advantages of boosting regimes (i.e. involving a second or further administration/immunisation) include raising the level of immune response in the subject, and/or increasing the duration of the immune response.

If a two dose regimen is required, e.g. for particular applications such as sustained immunity (e.g. in healthcare workers), ChAdOx1/MVA or ChAdOx1/RNA or ChAdOx1/protein as prime/boost regimes are preferred. More suitably if a two dose regimen is required, a homologous prime-boost regime is preferred such as ChAdOx1/ ChAdOx1, most suitably ChAdOx1 nCoV-19/ ChAdOx1 nCoV-19.

### Typical Modified RNA or Self-Amplifying mRNA Vaccination Regimen

Two doses of vaccine administered, typically 4-8 weeks between each dose

### Typical Protein Vaccination Regimen

Two or three doses of vaccine administered, typically 4-8 weeks between each dose and adjuvant must also be administered at immunisation

### Advantageous Viral Vector Vaccination Regimen According to the Invention

One dose of vaccine administered

In boost embodiments suitably the first administration comprises, or consists of, a composition according to the present invention comprising a viral vector capable of expressing the SARS-CoV2 Spike protein. Suitably the second or further (‘boost’) administration comprises exactly the same antigen as for viral vector. Suitably the second or further (‘boost’) administration comprises an RNA vaccine. Suitably the second or further (‘boost’) administration comprises a self amplifying RNA vaccine. Suitably the second or further (‘boost’) administration comprises IM administration. Suitably when the second or further (‘boost’) administration comprises adjuvant, said adjuvant is selected by the operator depending on platform. When the second or further (‘boost’) administration comprises saRNA no adjuvant needed. Suitably when the second or further (‘boost’) administration comprises RNA, the dose is suitably in the range of 0.001 to 1 microgrammes. Suitably when the second or further (‘boost’) administration comprises protein, the dose is suitably in the range of 1 to 15 microgrammes.

### Prime-Boost Doses

Participants included in the analysis were divided into groups which received two different dose levels as first dose (i.e. as first administration (prime)).

The doses of the first administration (prime) were
2.5 × 10¹⁰ vp (‘low dose’ / ‘half dose’ group) and
5.0 × 10¹⁰ vp (‘standard dose’ / ‘full dose’ group).

Thus in one embodiment the invention relates to a dual administration regime where a first administration and a second administration are given to a single subject, wherein the ratio of the dose of the first administration to the dose of the second administration is 0.5:1.

Thus in another embodiment the invention relates to a dual administration regime where a first administration and a second administration are given to a single subject, wherein the ratio of the dose of the first administration to the dose of the second administration is 1:1.

The combined analysis including all participants (n=11,636) resulted in an average efficacy of 70%. Participants who received a half dose as first dose (‘low dose’), followed by a full dose (‘standard dose’) at least one month later (n=2,741) (i.e. a ratio of the dose of the first administration to the dose of the second administration of 0.5:1) showed a vaccine efficacy of 90%.

In participants having received two standard doses (full doses), at least one month apart (n=8,895) (i.e. a ratio of the dose of the first administration to the dose of the second administration of 1:1), vaccine efficacy was 62%. The vaccine can be stored, transported and handled at normal refrigerated conditions (2-8° C./ 36-46° F.) for at least six months and administered within existing healthcare settings.

The invention also provides a composition as described above wherein administration of a first dose of said composition to a mammalian subject followed by administration of a second dose of said composition to said mammalian subject induces protective immunity in said subject.

The invention also provides a method of inducing an immune response against SARS-CoV2 in a mammalian subject, the method comprising
(i) administering a first dose of a composition as described above to said subject; and
(ii) administering a second dose of a composition as described above to said subject,
wherein said second dose comprises about twice the number of viral particles of said first dose.

The invention also provides a method of preventing SARS-CoV2 infection in a mammalian subject, the method comprising
(i) administering a first dose of a composition as described above to said subject; and
(ii) administering a second dose of a composition as described above to said subject,
wherein said second dose comprises about twice the number of viral particles of said first dose.

The invention also provides a composition for use as described above wherein said use comprises:
(i) administering a first dose of said composition to said subject; and
(ii) administering a second dose of said composition to said subject,
wherein said first dose and said second dose each comprise about the same number of viral particles.

The invention also provides a composition for use as described above wherein said use comprises:
(i) administering a first dose of said composition to said subject; and
(ii) administering a second dose of said composition to said subject,
wherein said second dose comprises about twice the number of viral particles of said first dose.

The invention also provides a method of inducing an immune response against SARS-CoV2 in a mammalian subject, or a method of preventing SARS-CoV2 infection in a mammalian subject, or a compoistion for use in such a method, the method comprising
(i) administering a first dose of a composition as described above to said subject; and
(ii) administering a second dose of a composition as described above to said subject,
wherein said first dose comprises about half the number of viral particles of said second dose.

The invention also provides a method of inducing an immune response against SARS-CoV2 in a mammalian subject, or a method of preventing SARS-CoV2 infection in a mammalian subject, or a compoistion for use in such a method, the method comprising
(i) administering a first dose of a composition as described above to said subject; and
(ii) administering a second dose of a composition as described above to said subject,
wherein the ratio of the number of viral particles in said first dose to the number of viral particles in said second dose is 0.5:1.

The invention also provides a method of inducing an immune response against SARS-CoV2 in a mammalian subject, or a method of preventing SARS-CoV2 infection in a mammalian subject, the method comprising
(i) administering a first dose of a composition as described above to said subject; and
(ii) administering a second dose of a composition as described above to said subject,
wherein the ratio of the number of viral particles in said first dose to the number of viral particles in said second dose is 1:2.

The inventors were very surprised by the beneficial technical effects delivered by the prime-boost immunisation regimens, and in particular the low dose - standard dose immunisation regimen (LD-SD) (i.e. low dose prime (0.5 x dose), standard dose boost (1.0 x dose)).

Suitably said second dose is administered at an interval of
a) less than 6 weeks,
b) 6 to 8 weeks,
c) 9 to 11 weeks, or
d) 12 weeks or more, after administration of said first dose.

In one embodiment suitably said first dose comprises about 2.5 × 10¹⁰ viral particles. (LD)

In one embodiment suitably said first dose comprises about 5 × 10¹⁰ viral particles. (SD)

Suitably said second dose comprises about 5 × 10¹⁰ viral particles. (SD)

In one embodiment suitably said first dose comprises about 2.5 × 10¹⁰ viral particles and said second dose comprises about 5 × 10¹⁰ viral particles. (LD-SD)

In one embodiment suitably said first dose comprises about 5 × 10¹⁰ viral particles and said second dose comprises about 5 × 10¹⁰ viral particles. (SD-SD)

Suitably said composition is administered by a route of administration selected from a group consisting of intranasal, aerosol, intradermal and intramuscular. More suitably said administration is intramuscular.

### Applications

The invention finds particular application in prevention or containment of outbreaks of SARS-CoV2. In this scenario, it is extremely advantageous to achieve protective immunity with only a single dose of vaccine. In the special considerations which apply to emerging pathogens such as SARS-CoV2, there is typically not time to give two doses. It is also exceptionally difficult to recall patients for their second dose. For example, patients may have many pressures on their time which can prevent attendance for a second dose. For example, they may have to travel from distance to receive a dose, or they may need to attend to their livelihoods which can prevent them from attending for more than a single dose. Thus, there is a need for a rapid onset of protection, which need is met by the present invention. The present invention also advantageously allows for avoidance of quarantine of patients in between doses which might otherwise be required since acquiring the infection in between doses would be potentially deleterious for the individual.

For similar reasons as outlined above, it is a technical benefit that the invention delivers protective immunity with only a single dose. Suitably the subject is a human. Suitably the method is a method of immunising. Suitably the immune response comprises a humoral response. Suitably the immune response comprises an antibody response. Suitably the immune response comprises a neutralising antibody response.

Suitably the immune response comprises a cell mediated response. Suitably the immune response comprises cell mediated immunity (CMI). Suitably the immune response comprises induction of CD8+ T cells. Suitably the immune response comprises induction of a CD8+ cytotoxic T cell (CTL) response. Suitably the immune response comprises both a humoral response and a cell mediated response. Suitably the immune response comprises protective immunity.

Suitably the composition is an antigenic composition. Suitably the composition is an immunogenic composition. Suitably the composition is a vaccine composition. Suitably the composition is a pharmaceutical composition. Suitably the composition is formulated for administration to mammals, suitably to primates, most suitably to humans.

Suitably the composition is formulated taking into account its route of administration. Suitably the composition is formulated to be suitable for the route of administration specified. Suitably the composition is formulated to be suitable for the route of administration selected by the operator or physician.

COVID19 is the disease caused by the SARS-CoV2 virus in humans. Suitably the invention further relates to a method for preventing COVID19 in a subject, the method comprising administering a composition as described above to said subject.

### Database Release

Sequences deposited in databases can change over time. Suitably the current version of sequence database(s) are relied upon. Alternatively, the release in force at the date of filing is relied upon.

As the skilled person knows, the accession numbers may be version/dated accession numbers. The citeable accession numbers for the current database entry are the same as above, but omitting the decimal point and any subsequent digits.

GenBank is the NIH genetic sequence database, an annotated collection of all publicly available DNA sequences (National Center for Biotechnology Information, U.S. National Library of Medicine 8600 Rockville Pike, Bethesda MD, 20894 USA; Nucleic Acids Research, 2013 Jan;41(D1):D36-42) and accession numbers provided relate to this unless otherwise apparent. Suitably the current release is relied upon. More suitably the release available at the effective filing date is relied upon. Most suitably the GenBank database release referred to is NCBI-GenBank Release 235: 15 Dec. 2019.

UniProt (Universal Protein Resource) is a comprehensive catalogue of information on proteins (‘UniProt: a hub for protein information’ Nucleic Acids Res. 43: D204-D212 (2015).). Suitably the current release is relied upon. More suitably the release available at the effective filing date is relied upon. Most suitably, the UniProt consortium European Bioinformatics Institute (EBI), SIB Swiss Institute of Bioinformatics and Protein Information Resource (PIR)’s UniProt Knowledgebase (UniProtKB) Release 2020_01 of 26-Feb-2020 is relied upon.

### Advantages

The invention possesses the advantage of protective immunity after single dose (single administration). Thus it is an advantage that the invention provides protective immune responses after only a single dose.

The phrase “protective immune response” or “protective immunity” as used herein means that the composition is capable of generating a protective response in a host organism, such as a human or a non-human mammal, to whom it is administered according to the invention. Suitably a protective immune response protects against subsequent infection or disease caused by SARS-CoV2.

In considering known vaccines from different disease areas, as noted above there exists a ChAdOx1-MERS spike protein viral vector vaccine (WO2018/215766). However, the inventors encountered problems when scaling up production of this MERS viral vector vaccine (which is not part of the invention). Firstly, the viral particles of this MERS vaccine could not be produced without Tet repression. Since at the point of scale up GMP quality cell lines are required for manufacture, obtaining a GMP quality cell line featuring Tet repression was not a straightforward matter. Moreover, the cell line was obtained at considerable expense. Thus, in addition to the extra labour and expertise required to operate the Tet repression system, the high cost of the cell line is a further drawback with this method. Even when virus was produced using this costly Tet repression system, the inventors observed further downstream problems of instability. It appeared to the inventors that possible slippage events in the nucleotide sequence might be contributing to the instability, since they observed that only 15% of the resulting virus was of the correct sequence/genetic structure. However, even when the inventors changed nucleotides in the coding sequence in an attempt to eliminate possible slippage-based instability, there were still problems in viral production - for example when the antigen was correct, the inventors observed deletions within the promotor sequence. Therefore, numerous serious technical difficulties were encountered in scaling up production of the ChAdOx1-MERS viral vectored vaccine. For all of these reasons, it was very surprising to the inventors when modifying the SARS-CoV2 spike protein gene expression cassette of the present invention and obtaining excellent yields with or even without Tet repression. In addition, the inventor was further surprised to note that Tet repression was not required for production of the viral vectored vaccine of the present invention. These outcomes were completely different to the inventor’s experience with the ChAdOx1-MERS viral vectored vaccine. The inventor was therefore greatly surprised by the technical properties of the viral vectored vaccine of the present invention, noting that experimental findings showed 6 out of 8 rescued viruses according to the present invention were of the completely correct genetic structure/sequence. This itself is a remarkable finding which astonished the inventor with the high stability of their new construct.

These advantages flow from the particular combination of features as set out in the claims.

For ChAdOx1 nCoV rapid approvals will be enabled.

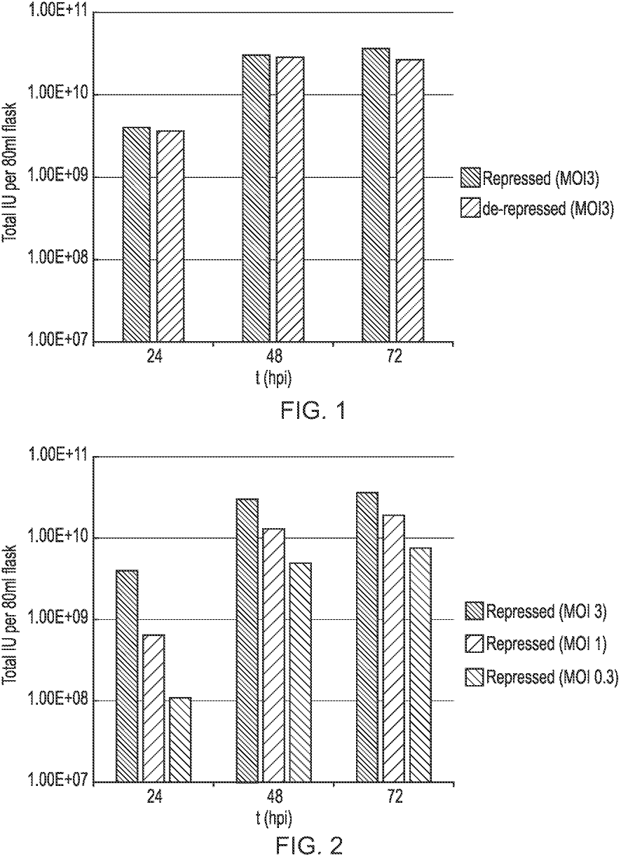

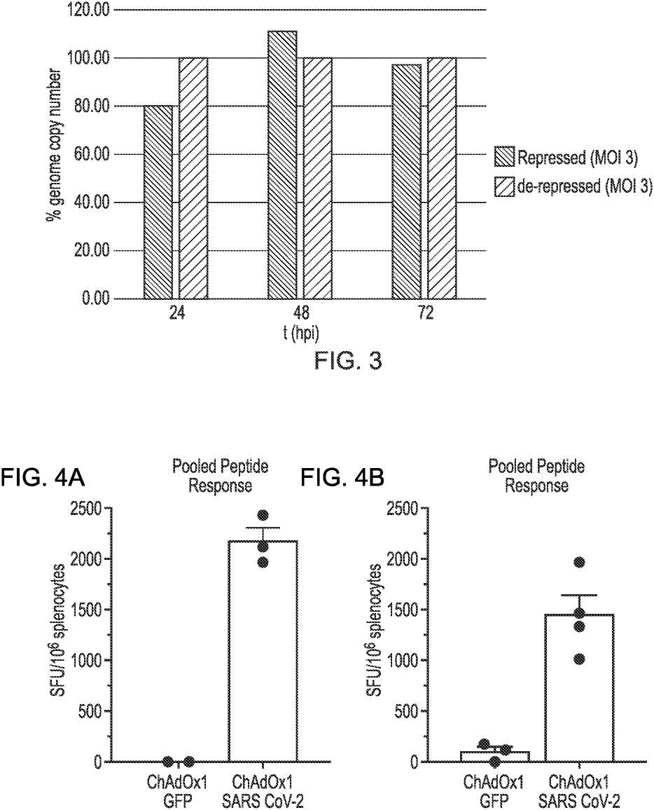

In addition it can be noted that the MERS vaccine (ChAdOx1-MERS spike protein viral vector vaccine (WO2018/215766)) has been tested in non-human primates (NHP). A MERS NHP challenge has been published (N. van Doremalen et al., (2020) Sci. Adv. 10.1126/sciadv.aba8399). This paper shows that the protection was only partial after one dose of the MERS vaccine, and two doses of the MERS vaccine were required for good protection. We refer to FIG. 2A of N. van Doremalen et al. which in particular shows that 2 doseswere required, and also FIG. 3B of N. van Doremalen et al.

### Spike Protein

The spike protein (S protein) is a large type I transmembrane protein. This protein is highly glycosylated, containing numerous N-glycosylation sites. Spike proteins assemble into trimers on the virion surface to form the distinctive “corona”, or crown-like appearance. The ectodomains of all CoV spike proteins share the same organization in two domains: a N-terminal domain named S1 that is responsible for receptor binding and a C-terminal S2 domain responsible for fusion. CoV diversity is reflected in the variable spike proteins (S proteins). Suitably the antigen is the SARS-CoV2 spike protein. Suitably the full length spike protein is used. Suitably full length means each amino acid in the spike protein is included. An exemplary spike protein is as disclosed in SEQ ID NO: 1.

It may be possible to use only the S1 domain of the spike protein, or only the soluble part of the spike protein, or only the receptor binding domain of the spike protein. However, most suitably according to the present invention the full length spike protein is used.

By choosing the full length spike protein, advantageously the correct confirmation of the protein in assured. Truncated proteins can assume unnatural conformations. This drawback is avoided by using the full length protein.

A further advantage of using the full length spike protein is that it allows for better T-cell responses. Without wishing to be bound by theory, it is believed that the more amino acid sequences present, then the more potential targets there are for the T-cell responses. Thus, suitably every amino acid of the wild type spike protein is included in the antigen of the invention.

### tPA

tPA (tissue plasminogen activator) - more specifically the tPA leader sequence - is suitably fused to the SARS-CoV2 spike protein antigen of the invention. Suitably tPA is fused to the N-terminus of the spike protein sequence.

Suitably tPA leader sequence means the tPA amino acid sequence of SEQ ID NO: 5

SEQ ID NO: 5

MDAMKRGLCCVLLLCGAVFVSASQEIHARFRR

In the above SEQ ID NO: 5 the C terminal ‘RR’ is not actually part of the tPA leader sequence. It comes from the fusion of two restriction sites. Suitably the tPA leader sequence may be used with or without the C terminal ‘RR’ e.g. SEQ ID NO: 7 or SEQ ID NO: 8. Most suitably the sequence is used as shown in SEQ ID NO: 5.

The underlined A is P in the naturally occurring tPA leader sequence. The P->A mutation has the advantage of improved antigen secretion.

Suitably the tPA leader sequence may be used with or without the P->A mutation. i.e. suitably the tPA leader sequence may be used as SEQ ID NO: 5 or SEQ ID NO: 6.

SEQ ID NO: 6

MDAMKRGLCCVLLLCGAVFVSPSQEIHARFRR

SEQ ID NO: 7 (=SEQ ID NO: 5 without C-terminal ‘RR’)

MDAMKRGLCCVLLLCGAVFVSASQEIHARF

SEQ ID NO: 8 (=SEQ ID NO: 6 without C-terminal ‘RR’)

MDAMKRGLCCVLLLCGAVFVSPSQEIHARF

More suitably the sequence is used with the P->A mutation (with or without the C terminal ‘RR’). Most suitably the sequence is used as shown in SEQ ID NO: 5.

An exemplary nucleotide sequence encoding tPA, which has been codon optimised for human codon usage, is as shown in SEQ ID NO: 9 (this is the sequence encoding SEQ ID NO: 5):

ATGGACGCCATGAAGAGGGGCCTGTGCTGCGTGCTGCTGCTGTGTGGCGC
CGTGTTTGTGTCCGCCAGCCAGGAAATCCACGCCCGGTTCAGACGG

It is believed that tPA promotes secretion of proteins to which it is fused. It is believed that tPA increases expression of proteins to which it is fused. Notwithstanding the underlying mechanism, the advantage in the invention of fusing tPA to the N-terminus of the spike protein antigen is that improved immunogenicity is achieved. Thus, most suitably the antigen of the invention is provided as a fusion with tPA. Most suitably the tPA is fused to the N-terminus of the spike protein antigen.

Suitably the antigen does not comprise any further sequence tags. Suitably the antigen does not comprise any further linker sequences.

### Adeno-Based Viral Vectors

Adenoviruses are attractive vectors for human vaccination. They possess a stable genome so that inserts of foreign genes are not deleted and they can infect large numbers of cells without any evidence of insertional mutagenesis.

Replication defective adenovirus can be engineered by deletion of genes from the E1 locus, which is required for viral replication, and these viruses can be propagated easily with good yields in cell lines expressing E1 from AdHu5 such as human embryonic kidney cells 293 (HEK 293 cells).

Previous mass vaccination campaigns in over 2 million adult US military personnel using orally administered live human adenovirus serotype 4 and 7 have shown good safety and efficacy data. Human adenoviruses are under development as vectors for malaria, HIV and hepatitis C vaccines, amongst others. They have been used extensively in human trials with excellent safety profile mainly as vectors for HIV vaccines.

A limiting factor to widespread use of human adenovirus as vaccine vectors has been the level of anti-vector immunity present in humans where adenovirus is a ubiquitous infection. The prevalence of immunity to human adenoviruses prompted the consideration of simian adenoviruses as vectors, as they exhibit hexon structures homologous to human adenoviruses. Simian adenoviruses are not known to cause pathological illness in humans and the prevalence of antibodies to chimpanzee origin adenoviruses is less than 5% in humans residing in the US.

Any suitable adeno-based viral vector may be used. In more detail, any replication-deficient viral vector, for human use preferably derived from a non-human adenovirus may be used. For veterinary use Ad5 may be used. ChAdOx2 is an example of a suitable non-human adenovirus vector for human use. Most suitably the adeno-based viral vector is ChAdOx1.

### ChAdOx1

ChAdOx1 is a replication-deficient simian adenoviral vector. Vaccine manufacturing may be achieved at small or large scale. Pre-existing antibodies to the vector in humans are very low, and the vaccines induce strong antibody and T cell responses after a single dose, whilst the lack of replication after immunisation results in an excellent safety profile in subjects of all ages.

ChAdOx1 is described in Dicks MDJ, Spencer AJ, Edwards NJ, Wadell G, Bojang K, et al. (2012) A Novel Chimpanzee Adenovirus Vector with Low Human Seroprevalence: Improved Systems for Vector Derivation and Comparative Immunogenicity. PLoS ONE 7(7): e40385, and in WO2012/172277. Both these documents are hereby incorporated herein by reference, in particular for the specific teachings of the ChAdOx1 vector, including its construction and manufacture.

For insertion of the nucleotide sequence encoding spike protein, suitably the E1 site may be used, suitably with the hCMV IE promoter. Suitably the short or the long version may be used; most suitably the long version as described in WO2008/122811, which is specifically incorporated herein by reference for the teaching of the promoters, particularly the long promoter. It is also possible to insert antigens at the E3 site, or close to the inverted terminal repeat sequences, if desired.

In addition, a clone of ChAdOx1 containing GFP is deposited with the ECACC: a sample of E. coli strain SW1029 (a derivative of DH10B) containing bacterial artificial chromosomes (BACs) containing the cloned genome of AdChOX1 (pBACe3.6 AdChOx1 (E4 modified) TIPeGFP, cell line name “AdChOx1 (E4 modified) TIPeGFP”) was deposited by Isis Innovation Limited on 24 May 2012 with the European Collection of Cell Cultures (ECACC) at the Health Protection Agency Culture Collections, Health Protection Agency, Porton Down, Salisbury SP4 0JG, United Kingdom under the Budapest Treaty and designated by provisional accession no. 12052403. Isis Innovation Limited is the former name of the proprietor/applicant of this patent/application.

### ChAdOx2

The nucleotide sequence of the ChAdOx2 vector (with a Gateway™ cassette in the E1 locus) is shown in SEQ ID NO. 2 This is a viral vector based on Chimpanzee adenovirus C68. (This is the sequence of SEQ ID NO: 10 in gb patent application number 1610967.0).

In addition, a clone of ChAdOx2 containing GFP is deposited with the ECACC: deposit accession number 16061301 was deposited by Isis Innovation Limited on 13 Jun. 2016 with the European Collection of Cell Cultures (ECACC) at the Health Protection Agency Culture Collections, Health Protection Agency, Porton Down, Salisbury SP4 0JG, United Kingdom under the Budapest Treaty. Isis Innovation Limited is the former name of the proprietor/applicant of this patent/application.

### ChAd63

In one embodiment a related vaccine vector, ChAd63, may be used if desired.

### Production of ChAdOx1 nCoV-19

ChAdOx1 nCoV-19 may be produced by any method known in the art. For example ChAdOx1 nCoV-19 may be produced as described in Example 10.

In overview, the spike protein (S) of SARS-Cov-2 (Genbank accession number YP_009724390.1) was codon optimised for expression in human cell lines and synthesised by GeneArt Gene Synthesis (Thermo Fisher Scientific). The sequence encoding amino acids 2-1273 were cloned into a shuttle plasmid following InFusion cloning (Clontech). The shuttle plasmid encodes a modified human cytomegalovirus major immediate early promoter (IE CMV) with tetracycline operator (TetO) sites, poly adenylation signal from bovine growth hormone (BGH) and a tPA signal sequence upstream of the inserted gene.

For the avoidance of doubt, “ChAdOx1 nCoV-19” means the ChAdOx1 adenoviral vector as described in Dicks et al. (2012) PLoS ONE 7(7): e40385, and/or in WO2012/172277, comprising the nucleotide sequence of SEQ ID NO: 4 (32 aa tPA leader fused to SARS-Cov-2 spike protein) inserted at the E1 locus of the ChAdOx1 adenoviral vector under the control of the CMV (cytomegalovirus) ‘long’ promoter. This is a preferred embodiment of the invention.

### Administration Route

In principle any suitable route of administration may be used. The invention may be administered by aerosol delivery to the respiratory tract using a widely available device commonly used for drug delivery. This may be a suitable route of vaccine delivery for respiratory pathogens such as coronaviruses. In one embodiment the composition may comprise a MVA-vectored vaccine, wherein aerosol delivery may result in strong immune responses in the respiratory tract at low doses. A further advantage of aerosol deliver is avoidance of needles. Suitably the route of administration is selected from group consisting of subcutaneous, intranasal, aerosol, nebuliser, intradermal and intramuscular. Suitably the route of administration is selected from a group consisting of intranasal, aerosol, intradermal and intramuscular. Suitably the route of administration is selected from a group consisting of intranasal, aerosol and intramuscular. More suitably the route of administration is selected from a group consisting of intranasal and intramuscular. Most suitably the route of administration is intramuscular. The route of administration may be applied to humans and/or other mammals.

### Dose

It should be noted that there are alternate ways of describing the dose for adenoviral vectors.
Viral particles - vp/mL. This refers to the count of total viral particles administered.
Infectious units - i.u./mL. This refers to the number of infectious units administered, and can be correlated more accurately with immunogenicity.

By convention, clinical trials in the UK tend to provide the dose in terms of viral particles.

Preferred doses according to the present invention are:

For humans, in one embodiment the range is from 10⁹ to 10¹¹ viral particles.

For humans, in one embodiment the range is from 2.5x 10¹⁰ vp to 5x 10¹⁰ vp.

For humans, in one embodiment the dose(s)/range of dose(s) may be derived from the examples below. Suitably no adjuvant is administered with the viral vector of the invention. Suitably the viral vector of the invention is formulated with simple buffer. An exemplary buffer may be as shown below under the heading ‘Formulation’.

### Further Features

Suitably the nucleic acid sequence is codon optimised for mammalian codon usage, most suitably for human codon usage.

Suitably a container containing a composition as described above is provided. Suitably said container may be a vial. Suitably said container may be a syringe.

Suitably a nebuliser containing a composition as described above is provided. Suitably a nasal applicator containing a composition as described above is provided. Suitably an inhaler containing a composition as described above is provided. Suitably a pressurised canister containing a composition as described above is provided.

A method of making a composition as described above is provided, said method comprising preparing a nucleic acid encoding the SARS-CoV2 spike protein, optionally fused to the tPA protein, and incorporating said nucleic acid into an adeno-based viral vector, suitably a ChAdOx1 vector. Suitably the nucleic acid is operably linked to a promoter suitable for inducing expression of said SARS-CoV2 spike protein (or SARS-CoV2 spike protein-tPA fusion protein) when in a mammalian cell such as a human cell.

### Formulation

Vaccine formulation may be liquid, suitably stable for at least 1 year at 2-8° C., or may be lyophilised, suitably stable at ambient temperatures e.g. room temperature 18-22° C.

The ChAdOx1 formulation buffer, as used for the clinical product is:

### Formulation Buffer Components

1. 10 mM Histidine
2. 7.5 % Sucrose
3. 35 mM Sodium chloride
4. 1 mM Magnesium chloride
5. 0.1 % Polysorbate 80
6. 0.1 mM EDTA
7. 0.5% Ethanol
8. Hydrochloric acid (for pH adjustment to ~pH 6.6)

Formulated in Water for Injection Ph Eur. Formulations for other administration routes such as aerosol will be adjusted accordingly by the skilled operator. Suitably the composition and/or formulation does not comprise adjuvant. Suitably adjuvant is omitted from the composition and/or formulation of the invention.

### Further Embodiments

As noted above, it may be possible to use only the S1 domain of the spike protein, or only the soluble part of the spike protein, or only the receptor binding domain of the spike protein.

In one embodiment, the spike protein may be provided as a truncated spike protein comprising the receptor binding domain (RBD) section of the spike protein. More suitably, the spike protein may be provided as a construct consisting essentially of the RBD part of the spike protein. More suitably, the spike protein may be provided as a construct consisting only of the RBD section of the spike protein.

Thus, in one embodiment only the receptor binding domain of the spike protein is used. Suitably this has the tPA fusion.

In this embodiment suitably the spike protein has the sequence of SEQ ID NO: 12, which presents the amino acid sequence of tPA-spike receptor binding domain (tPA sequence underlined): SEQ ID NO: 12

MDAMKRGLCCVLLLCGAVFVSASQEIHARFRRPNITNLCPFGEVFNATRF
ASVYAWNRKRISNCVADYSVLYNSASFSTFKCY GVSPTKLNDLCFTNVY
ADSFVIRGDEVRQIAPGQTGKIADYNYKLPDDFT GCVIAWNSNNLDSKV
GGNYNYLYRLFRKSNLKPFERDISTEIYQAGSTPC NGVEGFNCYFPLQS
YGFQPTNGVGYQPYRVVVLSFELLHAPATVCGPKKS TN.

In this embodiment suitably the nucleotide sequence encoding the spike protein has the sequence of SEQ ID NO: 13, which presents nucleotide sequence as revised by the inventor (i .e. after codon optimisation introduced runs of same bases and after those runs of same bases were revised to retain the same coding sequence but remove the repeats) encoding the SARS-CoV2 spike protein receptor binding domain with tPA (tPA encoding sequence underlined)

SEQ ID NO: 13:

ATGGACGCCATGAAGCGAGGCCTGTGCTGTGTTCTGCTTCTGTGTGGCGC CGTGTTTGTGTCCGCCAGCCAAGAGATCCACGCCAGATTTCGGAGACCG
AATATCACCAATCTGTGCCCATTCGGCGAGGTGTTCAATGCCACCAG AT
TCGCCTCTGTGTACGCCTGGAACCGGAAGCGGATCAGCAATTGCGTGG C
CGACTACTCCGTGCTGTACAACTCCGCCAGCTTCAGCACCTTCAAGTGC
TACGGCGTGTCACCTACCAAGCTGAACGACCTGTGCTTCACAAACGTGTA
CGCCGACAGCTTCGTGATCCGTGGAGATGAAGTGCGGCAGATTGCACCT
G GACAGACAGGCAAGATCGCCGACTACAACTACAAGCTGCCCGACGACT
TC ACCGGCTGTGTGATTGCCTGGAACAGCAACAACCTGGATAGCAAAGT
CGG CGGCAACTACAATTACCTGTACCGGCTGTTCCGGAAGTCCAATCTG
AAGC CCTTCGAGCGGGACATCTCCACTACAATTACCTGTACCGGCTGTT
CCGGAAGTCCAATCTGAAGCCCTTCGAGCGGGACATCTCCACCGAGATCT
ATC AGGCCGGCAGCACACCTTGTAACGGCGTGGAAGGCTTCAACTGCTA
CTTC CCACTGCAGTCCTACGGCTTTCAGCCCACAAATGGCGTGGGCTAC
CAGCC TTACAGAGTGGTGGTGCTGAGCTTCGAGCTGCTGCATGCTCCTG
CCACAG TGTGCGGCCCTAAGAAAAGCACCAATTAA

In one embodiment, the spike protein may be provided as a “pre-fusion form”. Thus, in one embodiment a ‘pre-fusion’ version of the spike protein is used. Suitably this has the tPA fusion. In this embodiment suitably the spike protein has the sequence of SEQ ID NO: 14, which presents amino acid sequence of tPA-spike prefusion protein (tPA sequence underlined)

SEQ ID NO: 14

MDAMKRGLCCVLLLCGAVFVSASQEIHARFRRFVFLVLLPLVSSQCVNLT
TRTQLPPAYTNSFTRGVYYPDKVFRSSVLHST QDLFLPFFSNVTWFHAI
HVSGTNGTKRFDNPVLPFNDGVYFASTEKSNII RGWIFGTTLDSKTQSL
LIVNNATNVVIKVCEFQFCNDPFLGVYYHKNNKS WMESEFRVYSSANNC
TFEYVSQPFLMDLEGKQGNFKNLREFVFKNIDGYF KIYSKHTPINLVRD
LPQGFSALEPLVDLPIGINITRFQTLLALHRSYLTP GDSSSGWTAGAAA
YYVGYLQPRTFLLKYNENGTITDAVDCALDPLSETKC TLKSFTVEKGIY
QTSNFRVQPTESIVRFPNITNLCPFGEVFNATRFASVY AWNRKRISNCV
ADYSVLYNSASFSTFKCYGVSPTKLNDLCFTNVYADSFV IRGDEVRQIA
PGQTGKIADYNYKLPDDFTGCVIAWNSNNLDSKVGGNYNY LYRLFRKSN
LKPFERDISTEIYQAGSTPCNGVEGFNCYFPLQSYGFQPTN GVGYQPYR
VVVLSFELLHAPATVCGPKKSTNLVKNKCVNFNFNGLTGTGV LTESNKK
FLPFQQFGRDIADTTDAVRDPQTLEILDITPCSFGGVSVITPG TNTSNQ
VAVLYQDVNCTEVPVAIHADQLTPTWRVYSTGSNVFQTRAGCLI GAEHV
NNSYECDIPIGAGICASYQTQTNSPGSASSVASQSIIAYTMSLGA ENSV
AYSNNSIAIPTNFTISVTTEILPVSMTKTSVDCTMYICGDSTECSN LLL
QYGSFCTQLNRALTGIAVEQDKNTQEVFAQVKQIYKTPPIKDFGGFN FS
QILPDPSKPSKRSFIEDLLFNKVTLADAGFIKQYGDCLGDIAARDLIC A
QKFNGLTVLPPLLTDEMIAQYTSALLAGTITSGWTFGAGAALQIPFAMQ
MAYRFNGIGVTQNVLYENQKLIANQFNSAIGKIQDSLSSTASALGKLQDV
VNQNAQALNTLVKQLSSNFGAISSVLNDILSRLDPPEAEVQIDRLITGR
L QSLQTYVTQQLIRAAEIRASANLAATKMSECVLGQSKRVDFCGKGYHL
MS FPQSAPHGVVFLHVTYVPAQEKNFTTAPAICHDGKAHFPREGVFVSN
GTH WFVTQRNFYEPQIITTDNTFVSGNCDVVIGIVNNTVYDPLQPELDS
FKEE LDKYFKNHTSPDVDLGDISGINASVVNIQKEIDRLNEVAKNLNES
LIDLQ ELGKYEQYIKWPWYIWLGFIAGLIAIVMVTIMLCCMTSCCSCLK
GCCSCG SCCKFDEDDSEPVLKGVKLHYT

In this embodiment suitably the nucleotide sequence encoding the spike protein has the sequence of SEQ ID NO: 15, which presents nucleotide sequence as revised by the inventor (i.e. after codon optimisation introduced runs of same bases and after those runs of same bases were revised to retain the same coding sequence but remove the repeats) encoding SARS-Cov2 spike prefusion protein with tPA (tPA sequence underlined)

SEQ ID NO: 15

ATGGACGCCATGAAGCGAGGCCTGTGCTGTGTTCTGCTTCTGTGTGGCGC CGTGTTTGTGTCCGCCAGCCAAGAGATCCACGCCAGATTTCGGAGATTC
GTGTTCCTGGTGCTGCTGCCACTGGTGTCTAGCCAGTGTGTGAACCT GA
CCACCAGGACACAGCTGCCTCCAGCCTACACCAACAGCTTCACCAGAG G
CGTGTACTACCCAGACAAGGTGTTCAGATCCAGCGTGCTGCACTCTACC
CAGGACCTGTTCCTGCCTTTCTTCAGCAACGTGACCTGGTTCCACGCCAT
CCACGTGTCC GGCACCAATGGCACCAAGAGATTCGACAACCCAGTGCTG
CCCTTCAACGA CGGAGTGTACTTTGCCAGCACCGAGAAGTCCAACATCA
TCAGAGGCTGGA TCTTCGGCACCACACTGGACAGCAAGACCCAGAGCCT
GCTGATCGTGAAC AACGCCACCAACGTGGTCATCAAAGTGTGCGAGTTC
CAGTTCTGCAACGA CCCATTCCTGGGCGTCTACTACCACAAGAACAACA
AGAGCTGGATGGAAA GCGAGTTCCGGGTGTACAGCAGCGCCAACAACTG
CACCTTCGAGTACGTG TCCCAGCCTTTCCTGATGGACCTGGAAGGCAAG
CAGGGCAACTTCAAGAA CCTGCGCGAGTTCGTGTTCAAGAACATCGACG
GCTACTTCAAGATCTACA GCAAGCACACACCTATCAACCTCGTGCGGGA
TCTGCCTCAGGGCTTCTCT GCTCTGGAACCACTGGTGGATCTGCCCATC
GGCATCAACATCACCCGGTT TCAGACACTGCTGGCCCTGCACAGAAGCT
ACCTGACACCTGGCGATAGCA GCTCTGGATGGACAGCTGGCGCCGCTGC
CTACTATGTGGGATACCTGCAG CCTCGGACCTTCCTGCTGAAGTACAAC
GAGAACGGCACCATCACCGACGC CGTGGATTGTGCTCTGGATCCTCTGA
GCGAGACAAAGTGCACCCTGAAGT CCTTCACCGTGGAAAAGGGCATCTA
CCAGACCAGCAACTTCCGGGTGCAG CCCACCGAATCCATCGTGCGGTTC
CCGAATATCACCAATCTGTGCCCATT CGGCGAGGTGTTCAATGCCACCA
GATTCGCCTCTGTGTACGCCTGGAACC GGAAGCGGATCAGCAATTGCGT
GGCCGACTACTCCGTGCTGTACAACTCC GCCAGCTTCAGCACCTTCAAG
TGCTACGGCGTGTCACCTACCAAGCTGAA CGACCTGTGCTTCACAAACG
TGTACGCCGACAGCTTCGTGATCCGTGGAG ATGAAGTGCGGCAGATTGC
ACCTGGACAGACAGGCAAGATCGCCGACTAC AACTACAAGCTGCCCGAC
GACTTCACCGGCTGTGTGATTGCCTGGAACAG CAACAACCTGGATAGCA
AAGTCGGCGGCAACTACAATTACCTGTACCGGC TGTTCCGGAAGTCCAA
TCTGAAGCCCTTCGAGCGGGACATCTCCACCGAGATCTATCAGGCCGGCA
GCACACCT TGTAACGGCGTGGAAGGCTTCAACTGCTACTTCCCACTGCA
GTCCTACGG CTTTCAGCCCACAAATGGCGTGGGCTACCAGCCTTACAGA
GTGGTGGTGC TGAGCTTCGAGCTGCTGCATGCTCCTGCCACAGTGTGCG
GCCCTAAGAAA AGCACCAATCTCGTGAAGAACAAATGCGTGAACTTCAA
CTTCAACGGCCT GACCGGCACCGGCGTGCTGACAGAGAGCAACAAGAAG
TTCCTGCCATTCC AGCAGTTCGGCCGGGATATCGCCGATACCACAGATG
CCGTCAGAGATCCT CAGACACTGGAAATCCTGGACATCACACCTTGCAG
CTTCGGCGGAGTGTC TGTGATCACGCCTGGCACCAACACCAGCAATCAG
GTGGCAGTGCTGTACC AGGACGTGAACTGTACCGAAGTGCCCGTGGCCA
TTCACGCCGATCAGCTG ACACCTACATGGCGGGTGTACTCCACCGGCAG
CAATGTGTTTCAGACCAG AGCCGGCTGTCTGATCGGAGCCGAGCACGTG
AACAATAGCTACGAGTGCG ACATCCCTATCGGCGCTGGCATCTGCGCCT
CTTACCAGACACAGACAAAC AGCCCTGGCAGCGCCTCTTCTGTGGCCAG
CCAGAGCATCATTGCCTACAC AATGTCTCTGGGCGCCGAGAACAGCGTG
GCCTACTCCAACAACTCTATCG CTATCCCGACCAACTTCACCATCAGCG
TGACCACAGAGATCCTGCCTGTG TCCATGACCAAGACCAGCGTGGACTG
CACCATGTACATCTGCGGCGATTC CACCGAGTGCTCCAACCTGCTGCTG
CAGTACGGCAGCTTCTGCACCCAGC TGAATAGAGCCCTGACAGGGATCG
CCGTGGAACAGGACAAGAACACCCAA GAGGTGTTCGCCCAAGTGAAGCA
GATCTACAAGACGCCTCCTATCAAGGA CTTCGGCGGCTTCAATTTCAGC
CAGATTCTGCCCGATCCTAGCAAGCCCA GCAAGCGGAGCTTCATCGAGG
ACCTGCTGTTCAACAAAGTGACACTGGCC GACGCCGGCTTCATCAAGCA
GTATGGCGATTGTCTGGGCGACATTGCCGC CAGGGATCTGATTTGCGCC
CAGAAGTTTAACGGACTGACAGTGCTGCCTC CTCTGCTGACCGATGAGA
TGATCGCCCAGTACACATCTGCCCTGCTGGCCGGCACAATCACAAGCGGC
TGGACA TTTGGAGCTGGCGCTGCCCTGCAGATCCCATTTGCTATGCAGA
TGGCCTA CCGGTTCAACGGCATCGGAGTGACCCAGAATGTGCTGTACGA
GAACCAGA AGCTGATCGCCAACCAGTTCAACAGCGCCATCGGCAAGATC
CAGGACAGC CTGAGCAGCACAGCAAGCGCCCTGGGAAAGCTGCAGGACG
TGGTCAACCA GAATGCCCAGGCACTGAACACCCTGGTCAAGCAGCTGTC
TAGCAACTTCG GCGCCATCAGCTCTGTGCTGAACGATATCCTGAGCAGA
CTGGACCCTCCT GAAGCCGAGGTGCAGATCGACAGACTGATCACCGGAA
GGCTGCAGTCCCT GCAGACCTACGTTACCCAGCAGCTGATCAGAGCCGC
CGAGATTAGAGCCT CTGCCAATCTGGCCGCCACCAAGATGTCTGAGTGT
GTGCTGGGCCAGAGC AAGAGAGTGGACTTTTGCGGCAAGGGCTACCACC
TGATGAGCTTCCCTCA GTCTGCGCCTCACGGCGTGGTGTTTCTGCACGT
GACATACGTGCCCGCTC AAGAGAAGAATTTCACCACCGCTCCAGCCATC
TGCCACGACGGCAAAGCC CACTTTCCTAGAGAAGGCGTGTTCGTGTCCA
ACGGCACCCATTGGTTCGT GACCCAGCGGAACTTCTACGAGCCTCAGAT
CATCACCACCGACAACACCT TCGTGTCTGGCAACTGCGACGTCGTGATC
GGCATTGTGAACAATACCGTG TACGACCCTCTGCAGCCCGAGCTGGACA
GCTTCAAAGAGGAACTGGATAA GTACTTTAAGAACCACACAAGCCCTGA
CGTGGACCTGGGCGATATCAGCG GAATCAATGCCAGCGTCGTGAACATC
CAGAAAGAGATCGACCGGCTGAAC GAGGTGGCCAAGAATCTGAACGAGA
GCCTGATCGACCTGCAAGAACTGGG CAAGTACGAGCAGTACATCAAGTG
GCCCTGGTACATCTGGCTGGGCTTTA TCGCCGGACTGATTGCCATCGTG
ATGGTCACAATCATGCTGTGTTGCATG ACCAGCTGCTGTAGCTGCCTGA
AGGGCTGTTGTAGCTGTGGCTCCTGCTG CAAGTTCGACGAGGACGATTC
TGAGCCCGTGCTGAAGGGCGTGAAACTGC ACTACACCTGATAA

### Sequence Variation

Suitably the sequence is, or is derived from, amino acid sequence provided herein, such as SEQ ID NO. 1. A degree of sequence variation may be tolerated. Suitably the sequence used in the vector of the invention comprises amino acid sequence having at least 99% sequence identity to the reference amino acid sequence, for example the reference amino acid sequence provided as SEQ ID NO. 1.

The sequence identity level of 99% compared to SEQ ID NO. 1 (having 1272 amino acids) corresponds to approximately 12 to 13 substitutions across the full length of the spike protein sequence provided as SEQ ID NO. 1. Suitably the spike protein construct used has 13 or fewer substitutions relative to SEQ ID NO: 1, suitably 12 or fewer substitutions relative to SEQ ID NO: 1, suitably 10 or fewer substitutions relative to SEQ ID NO: 1, suitably 8 or fewer substitutions relative to SEQ ID NO: 1, suitably 6 or fewer substitutions relative to SEQ ID NO: 1, suitably 4 or fewer substitutions relative to SEQ ID NO: 1, suitably 2 or fewer substitutions relative to SEQ ID NO: 1, suitably one substitution relative to SEQ ID NO: 1.

Suitably any amino acid substitutions are not in the receptor binding domain. Suitably any amino acid substitutions are outside the receptor binding domain. Suitably counting of substitutions does not include addition of the tPA sequence.

### MVA - SARS-CoV2 Spike Protin

We disclose an MVA vector carrying the SARS-CoV2 spike protein. The MVA vector described herein features a mH5/F11 promoter system in one embodiment, or relies on a standard F11 promoter in another embodiment. In any case, these promoter systems are known in the art, for example in published patent US 9, 273, 327B2 (Cottingham - granted 1 Mar. 2016 - ‘Poxvirus Expression System’) - this document is hereby incorporated by reference, in particular for the specific teachings of promoter(s) for use herein.

In the context of the present invention, MVA vector delivering SARS-CoV2 spike protein is taught as a useful optional boost in an immunisation regimen as described. The first dose should preferably be ChAdOx1-SARS-CoV2 spike protein (most preferably comprising the tPA fusion to the N-terminus of the spike protein) and the optional second administration preferably comprises MVA-SARS-CoV2 spike protein.

As will be apparent, the main focus of the invention is in provision of a single dose SARS-CoV2 vaccine. However, in this embodiment featuring a second (boosting) administration, preferably the second (boosting) administration is in a different viral vector i.e. a heterologous “prime-boost” regime. Suitably the second (boosting) administration comprises a MVA vector. This finds particular application for example in inducing immunity in subjects such as healthcare workers. It is a particular problem that healthcare workers can contract a SARS-CoV2 infection. Since they are typically in good health themselves, this has very little effect, if any, on their general health. However, when they are infected they can of course excrete virus, which can go on to infect immune compromised patients in their care with disastrous consequences. Therefore, there is a special and particular problem in the immunisation of healthcare workers. A durable and long lasting immunity is desired for these professionals. Therefore, whilst it is a core tenet of the invention that a single dose of vaccine provides protection against SARS-CoV2 infection, in the special case of healthcare workers the protective immunity is desired to last as far as possible into the future. For most applications, a temporary immunity (‘temporary’ contrasted with a lifelong immunity) is entirely adequate to protect the individual and/or to halt the spread of the infection. However, in the special case of healthcare workers any way of extending their immunity in time is itself additionally advantageous. In this scenario, we teach a “prime-boost” regimen comprising a first administration of an adenoviral vector- SARS-CoV2 composition such as a ChAdOx- SARS-CoV2 composition, followed by a second (boosting) administration of a viral vector comprising the SARS-CoV2 spike protein, such as a MVA vector expressing the SARS-CoV2 spike protein. Thus, in the inventors’ opinion, MVA- SARS-CoV2 spike protein has limited use but may find particular application as a heterologous boost following a ChAdOx- SARS-CoV2 spike protein priming vaccination. In one embodiment the order of immunisations may be reversed so that the MVA- SARS-CoV2 vaccine is administered first followed by the ChAdOx- SARS-CoV2 vaccine after an interval of typically 1 - 8 weeks.

Thus in one aspect the invention provides a method of inducing an immune response against SARS-CoV2 in a mammalian subject, the method comprising
(i) administering a composition comprising a viral vector, the viral vector comprising nucleic acid having a polynucleotide sequence encoding the spike protein from SARS-CoV2, characterised in that said viral vector is an adenovirus based vector to said subject, and
(ii) administering a composition comprising a viral vector, the viral vector comprising nucleic acid having a polynucleotide sequence encoding the spike protein from SARS-CoV2, characterised in that said viral vector is a MVA based vector to said subject.

Suitably step (i) is a priming composition.

Suitably step (ii) is a boosting composition.

Suitably step (ii) is carried out 1-8 weeks after the step (i), most suitably 4 weeks after step (i).

Further particular and preferred aspects are set out in the accompanying independent and dependent claims. Features of the dependent claims may be combined with features of the independent claims as appropriate, and in combinations other than those explicitly set out in the claims.

Where an apparatus feature is described as being operable to provide a function, it will be appreciated that this includes an apparatus feature which provides that function or which is adapted or configured to provide that function.

### EXAMPLES

### Vaccine Development Endpoint / Summary

ChAdOx1 SARS-CoV2 vaccine for preclinical studies is generated
Preclinical testing includes initial immunogenicity studies in mice followed by efficacy studies in ferrets and NHPs
A vaccine seed stock suitable for cGMP manufacture is generated
A phase I/II batch of 1000 doses vaccine is manufactured to cGMP and made available for clinical trials
Ethical and regulatory approval for a UK trial is obtained
A Phase I/II study is conducted, providing safety and immunogenicity data in adults, older adults and children
Large scale manufacture of vaccine (200 L, estimated 20,000 doses per batch) is initiated

### Overview

In these examples the inventors:
1. Demonstrate ChAdOx1 nCoV vaccine efficacy in ferret and non-human primate challenge models
2. Produce 1000 doses of ChAdOx1 SARS-CoV2 ready for use in clinical studies
3. Conduct a clinical trial of ChAdOx1 SARS-CoV2 in adults aged 18-50, then progressing to adults over 50 years and school age children
4. Characterise the immune response to SARS-CoV2 Spike protein in clinical trial volunteers

This vaccine against SARS-CoV2 is used to demonstrate clinical development and pre-clinical efficacy studies. Other vaccine technologies such as recombinant protein, DNA and RNA vaccines are in development, but require multiple doses to achieve measurable immune responses to the vaccine antigen. Based on pre-existing preclinical data, the inventors assert that the vaccines of the invention, most suitably ChAdOx1-nCoV as described herein, are able to induce protective immunity after a single dose, and within 14 days of the (suitably first and only) vaccination.

### Example 1

For ChAdOx1 SARS-CoV2, vaccine seed stock preparation is carried out. Development of a rapid vaccine seed stock generation method is initiated, with the aim of rapid response in an outbreak situation. In emergency situations work is accelerated to allow rapid production of ChAdOx1 SARS-CoV2 vaccine seed stock in parallel with research grade material for pre-clinical testing. An important component of the ‘rapid method’ is the adoption of rapid vaccine release testing protocols to reduce time for vaccine release testing from 5 months to 1 month. Outline plans have already received a positive review from the MHRA.

Further discussions are undertaken with the MHRA on the Product Specification to be employed for ChAdOx1 SARS-CoV2. Suitably rapid ethical and regulatory approval for UK trials can be arranged within days of the Clinical Trial Authorisation (CTA) submission.

Manufacture is then transferred to GMP manufacturers. Suitably one manufacturer (Advent) produces material (initially 1000 doses) for a phase I/II study in the UK, testing the vaccine in adults, then progressing to older adults and children, using a single dose at a level determined by earlier ChAd vaccine studies.

Suitably one manufacturer (CanSino) manufactures in China, at 200 L scale, 20,000 doses per batch. Further studies to determine vaccine efficacy and enable vaccine licensure will be carried out depending on clinical need e.g. the development of epidemic event(s). If required, production can be further optimised and capacity extended, to both increase the yield per batch and the number of batches that can be produced simultaneously. High capacity filling lines are already available, with or without lyophilisation.

Preclinical studies to test vaccine immunogenicity and efficacy in a non-human primate model are conducted. Suitably this is conducted at NIH. Suitably further work on correlates of protection in this model is carried out.

### Example 2

The inventors produce a ChAdOx1-vectored vaccine against nCoV-2019. The inventors teach complete GMP manufacture of a first batch for clinical studies. Animal efficacy studies are conducted by two organisations, NIH and CSIRO, to demonstrate efficacy of the vaccine after a single dose in both non-human primates and ferrets.

Ferrets (or in a separate experiment, non-human primates) are vaccinated with either one dose of ChAdOx1 nCV-19, or two doses given at 4 week intervals. Control animals will be vaccinated with either two dose or two doses of a control vaccine, ChAdOx1 expressing green fluorescent protein (GFP). 4 weeks after the final vaccination the animals are challenged by exposure to live SARS-CoV2 virus via the respiratory tract.

Animals will be monitored for clinical signs, and samples from the respiratory tract will be taken daily to determine the extent of virus important to note that these assay were conducted at either 9 or 10 days post vaccination whereas it would be usual to assay T cell responses at 14 days and antibodies at 28 days. These are exceptionally strong responses for such an early assay timepoint. This is of particular importance for a vaccine to be used against an outbreak pathogen for which rapid onset of immunity will be highly beneficial.

The inventors disclose a phase I/II clinical trial with an adaptive design that allows further groups to be added following demonstration of safety and immunogenicity in the First in Human component of the study. In parallel, vaccine seed stock is provided to a manufacturer with large scale manufacturing capability and a proven track record in manufacturing adenovectors to cGMP, in order to enable supply of large numbers of doses for efficacy testing and deployment.

The phase I/II trial described here may be conducted in the UK. This provides safety and immunogenicity data in adults, older adults (who are at highest risk of morbidity and mortality) and children (who may be responsible for much transmission of any respiratory pathogen). The next stages of clinical development depend on the progress of a particular outbreak, with the data generated allowing for further studies.

### Example 3

The order of events for preclinical studies is first to demonstrate immunogenicity in mice (antibodies and T cell responses), and then to proceed with vaccine efficacy testing in non-human primates (in collaboration with NIH), and vaccine efficacy testing in ferrets (in collaboration with CSIRO). CEPI is already funding CSIRO in establishing the ferret model for 2019-nCoV (PI:Prof.S.S.Vasan).

The inventors teach that the vaccine is protective after a single dose, and the data demonstrating this are generated as above. An efficacy study with 6-8 ferrets in parallel to the natural progression study (planned challenge date 23 March), is in motion.

Clinical dose ranging has been established in other studies. In the First in Human component of the clinical studies we test two different doses initially. Given the clinical experience with ChAdOx1-vectored vaccines a toxicology study may not be required for the UK study. However, a toxicology study is conducted as has been done for other ChAdOx1-vectored vaccines (conducted even if not required), as the data are considered valuable for initiating studies in other countries.

The potency assay is well established and is a measure of vaccine concentration rather than requiring any immunology studies. Below is a protocol for determining adenovirus infectivity which is useful as the potency assay for adeno vectored vaccines:
1.0 Jenner Laboratory Protocol Number J259
2.0 Version Number 9
3.0 Adenovirus Titre Immunoassay
4.0 Notes:

This method differs from the previous version in that it measures 4 viruses in triplicate on each plate. Each plate requires the single preparation of all four viruses using a 12 channel multipipette.

This assay is very susceptible to cell loss from the monolayers during the immunostaining protocol. It also appears to be sensitive to edge effect both during cell culture and staining. Specifically: HEK293 cells are only loosely adherent. We use coated plates to try and overcome this but the monolayers are still relatively fragile.

Take care with pipetting throughout the protocol (cell culture and staining)

### Edge Effect During Cell Culture

Evaporation from the edge wells and particularly the corner wells can negatively affect cell health in these wells (see note in appendix)

If the volume is too low, the meniscus that forms can both concentrate non-adhered calls to the edges and leave those cells in the centre of the wells with insufficient media covering them.

Use the Nunc square boxes with damp blotting paper to provide a constant high humidity atmosphere. Plates placed near the incubator door and/or removed frequently for observation will be subjected to drops in humidity and increased evaporation.

### Edge Effect During Staining

When plates are transferred between different temperatures the edges will alter temperature much more rapidly than the centre wells. It is important to allow the entire plate to reach the same temperature at each step. Avoid stacking plates as those in the centre will be at very different temperatures to the outer plates. Ensure incubation times are long enough to bring the plate to uniform temperature.
Other Documents
   MSDS refer to MSDS for the relevant safety information on the individual reagents
      \\Imsnw3_jenner_server\jenner\hill group\Safety\COSHH assessments\Manufacturers material safety data sheets
   R002 Adenovirus
      \\Imsnw3_jenner_server\ienner\hill_group\Safety\GMO RA\R002 adenovirus.doc
   R004 GMO RA appendix
   C030 Culture of primary cells and cell lines including freezing and reviving
   C024 Use of penicillin for tissue culture by sensitised individuals
   C066 Use of antibiotics for selection of cells
   J011 Passaging 293 cells

Safety glasses or over-glasses must be worn when washing 96-well plates during the staining process.

There is a risk that eye splashes may occur when flicking the fluid from the plates into the sink.

5.0 Definitions
FCS Foetal calf serum
DMEM Dulbecco’s modified Eagle’s media
PBS Phosphate buffered saline
TBS Tris buffered saline
RT Room temperature

6.0 Objective
To calculate the infectious viral titre by visualizing immunostained infectious cells.

7.0 Reagents
DMEM Sigma D6546
DMEM Lonza/ SLS LZBE12-604F
Pen/Strep Sigma
Glutamine Sigma
FCS Sigma F2442
Trypan blue stain Sigma T8154
TrypLE Express Invitrogen I2605
Plate 96 well black/clear BD purecoat amine VWR 734-1476
Methanol Sigma 32213
PBS tablets Sigma P4410-100TAB
Dulbecco’s Phosphate-Buffered Saline powder (10L) Invitrogen 21600-069
Bloxall Vector labs SP-6000
1000x primary antibody (1 mg/ml) 500 µl AbCam ab7428
(£235/500 plates) - 47 p per plate
10000x Anti-Mouse IgG (whole molecule)-Alkaline Phosphatase antibody produced in goat Sigma A3562
(£63/500 plates) - 13 p per plate
TBS Sigma T5912-1L
BCIP/NBT(Plus) solution Europa Bioproducts Ltd MO711A-1000
1% casein ready made solution ThermoScientific 37528
(£94/litre)
Equipment
Class II BioSafety Cabinet Scanlaf Mars
CO₂ incubator RS Biotech Galaxy R
37° C. water bath Grant SUB6
Microscope Leica DMIL
Neubauer haemocytometer
Plate 96 well black/clear bd purecoat amine VWR 734-1476
(£5.60 per plate)
96-well v-bottomed tissue culture plate Greiner Bio-one 651201
Aspirator
Vortex
2 x p200 8 well multichannel pipette
p1000 pipette
p10 pipette
Nunc square box
Filter paper ThermoScientific 86620

8.0 Method
Preparation of reagents:
   Complete 10% FCS DMEM+Blasticidin
   500 ml DMEM
   50 ml ml FCS (10% final conc)
   5 ml Pen/strep (100 U Penicillin, 0.1 mg strep ml⁻¹ final conc)
   10 ml L-glutamine (4 mM final conc)
   250 µl Blasticidin (5 ug/ml final conc)

Day 0 96 well plates should be seeded from TREX cells taken from a T175 Flask grown up to 50%-60% confluency.

### Day 1

### 9.1 Plating Cells - Viruses are Usually Titred on HEK293-TRex Cells Irrespective of the Cell Line Used For Poduction. Prepare Sufficient Plates for Each Request at the Densities Shown Below

9.1.1. Cells should be counted by a glass haemocytometer.

9.1.2. Pre-warm Nunc box containing blotting paper wetted with water/Sigmaclean and plates.

9.1.3. 100 µl of cells seeded per well - (5 x 10⁶ cells in 12 ml per plate).

9.1.4. Place plates in the Nunc square box with some damp blotting paper underneath to provide constant, high humidity environment.

9.1.5. Incubate plates overnight in 37° C., 5% CO₂ incubator

9.1.6. Remove an aliquot of 10% DMEM (10 mL per virus plus 10 mL for the control) to equilibrate to room temperature overnight.

### Day 2

Before starting any work, check that the cells in the 96 wp are 100% confluent. They must be 100% confluent to capture all of the virus particles in the sample. Plates may be left until the afternoon but must not be left an additional day.

### 9.2 Preparation of Serial Dilution of Virus

9.2.1. Pre-warm Nunc box containing wetted blotting paper in the virus lab.

9.2.2. Remove one 10 µl aliquot of purified virus from the -80° C. freezer and thaw at RT.

9.2.3. Top up to 100 ul with 90 ul D10 + Blasticidin.

9.2.4. Wrap lid of each virus aliquot with parafilm.

9.2.5. Sonicate each aliquot for 30 seconds at 50% power.

9.2.6. Ensure that you use good pipetting technique as described in J212 to make sure data of the highest quality possible is achieved.

9.2.7. Transfer 20 ml media (10% FBS, DMEM + blasticidin) into reagent reservoir.

9.2.8. Flick the aliquots of virus to thoroughly resuspend the virus and then force the material to the base of the tube with a large flick.

9.2.9. For this method, you will prepare the dilutions using two v-bottomed plates, oriented with the 12-wells vertically.

9.2.10. Take 20 µl of sample from the stock tube and add to the first column of a 96 v-bottom plate.

9.2.11. Repeat steps 9.2.7 and 9.2.8 for the remaining viruses to add to the plate.

9.2.12. Using a multichannel pipette, add 180 µl media to the wells.

9.2.13. Using fresh tips on the p200 multichannel; triturate the samples ten times slowly to avoid foaming.

9.2.14. Use a multichannel pipette to transfer 20 µl of the first dilution into the second column of wells.

9.2.15. Continue to prepare the dilution series as shown in the table below. Note that the volumes to add change half way across the table.

| | Virus stock | 10⁻² | 10-³ | 10⁻⁴ | 10⁻⁵ | 10⁻⁶ | 3.3 x10⁻⁷ | 1.1 x10⁻⁷ | 3.7 x10⁻⁸ | 1.2 x10⁻⁸ | 4.1 x10⁻⁹ |
|---|---|---|---|---|---|---|---|---|---|---|---|
| Virus (µl) | Stock | 20 | 20 | 20 | 20 | 20 | 75 | 75 | 75 | 75 | 75 |
| 10% DMEM (µl) | - | 180 | 180 | 180 | 180 | 180 | 150 | 150 | 150 | 150 | 150 |
| | | | Plate 1 | | | | Plate 2 | | | | |

### 9.3 Preparation of Serial Dilution of Control Virus

9.3.1. Remove one 10 µL aliquot of control virus from the -80° C. freezer and thaw at RT.

9.3.2. Ensure that you use good pipetting technique as described in J212 to make sure data of the highest quality possible is achieved.

9.3.3. Flick the aliquots of virus to thoroughly resuspend the virus and then force the material to the base of the tube with a large flick.

9.3.4. Prepare 4 cryovials, labelled -2, -4, -6, -8

9.3.5. Take 5 µl of sample from the stock tube and add to the first 2 mL cryovial.

9.3.6. Add 495 µl media to the cryovial.

9.3.7. Using a fresh tip, triturate the samples ten times slowly to avoid foaming.

9.3.8. Transfer 5 µl of the first dilution into a second cryovial.

9.3.9. Continue to prepare the dilution series as shown in the table below.

| | 10⁻² | 10⁻⁴ | 10⁻⁶ | 10⁻⁸ |
|---|---|---|---|---|
| Virus (µL) | 5 | 5 | 5 | 5 |
| 10% DMEM (µL) | 495 | 495 | 495 | 495 |

### 9.4 Infection of Cells with Serial Dilution of Virus

9.4.1 Using the multichannel aspirating pipette, aspirate media from one half of a 96 well plate prepared the previous day- take care not to damage the cell monolayer with the pipette tips. You will need to touch the well bases but just take as much care as possible.

9.4.2 Carefully, so as not to damage the cell layer, add 50 µl of each of the above dilutions per well to the side wall of the well.

9.4.3 Add 50 µl of media to the negative control wells.

9.4.4 Place plates in the Nunc square tray with damp blotting paper in the base to provide a constant, high humidity environment.

9.4.5 Incubate plates for 24 hours at 37° C., 5% CO₂.

### Day 3

### 9.5 Addition of Media to Wells to Ensure Cell Health- Work Should Commence Exactly 24 Hours After Infection wth Virus

9.5.1 Pre-warm media to 37° C.

9.5.2 Using a multichannel pipette, add 50 µl of 10% DMEM + blasticidin to each well of the plates

9.5.3 Incubate plates overnight in 37° C., 5% CO₂ incubator

9.5.4 Place methanol in freezer at -20° C. for use on day 4

### Day 4

### 9.4 Fixing Cells - Work Should Commence Exactly 48 Hours After Infection with Virus

9.4.1. Check the positive control wells for GFP expression and count the wells using the fluorescence microscope.

9.4.2. Using multichannel aspirator, aspirate media from 96 well plate, one plate at a time.

9.4.3. Take care not to damage the cell monolayer with the pipette.

9.4.4. Very gently, using a multichannel pipette, add 100 µl of pre-chilled methanol to all wells.

9.4.5. Incubate plates at -20° C. in virus lab for a minimum of 20 minutes.

NOTE: Protocol can be stopped and plates stored at -20° C. until required.

### 9.5 Immunostaining - Wear Safety Glasses/Over-Glasses for Wash Steps

9.5.1. Remove the plates from -20° C. and incubate at room temperature for 10 minutes (do not stack).

9.5.2. Prepare 1 x Tris buffered saline solution in a 1 litre Duran bottle by diluting 100 ml of the 10 x solution to 1 litre with 15 MΩ water.

9.5.3. Prepare 3% Marvel in TBS: Weigh 1.2 g of Marvel into a 50 ml falcon tube, make up to 40 ml with TBS and leave at RT.

9.5.4. Wash plates 4 times with RT D-PBS (Immerse plates in D-PBS in a sandwich box and flick into the sink, blot on blue roll. Wash and flick a further 3 times. Finally blot on blue roll to ensure all PBS is removed). 9.5.5. Pipette 12 ml of bloxall from the bottle into a reagent reservoir. Add 100 µl of reagent to each well using the repeater-multichannel. Block for 30 minutes at RT* - do not stack plates.

*Bloxall is supplied in a light-tight dropper bottle. Remove the dropper cap before first use to allow pipetting and transfer into a reagent reservoir.

9.5.6. Remove Bloxall by flicking in sink.

9.5.7. Wash plates 4 times with RT D-PBS (Immerse plates in D-PBS in a sandwich box and flick into the sink, blot on blue roll. Wash and flick a further 3 times. Finally blot on blue roll to ensure all PBS is removed). 9.5.8. Pipette 25 ml of casein solution in PBS to a reagent reservoir. Add 200 µl or reagent to each well and block for 15 min. at RT.

9.5.9. Dilute primary antibody 1:1000 in 1% casein solution (12 µl in 12 ml per plate).

9.5.10. Remove casein by flicking in the sink (there is no need to wash at this step) blot on blue roll to ensure all casein solution is removed from wells (any residual casein will dilute the primary antibody added in the next step).

9.5.11. Add 100 µl of 1 x anti-Hexon antibody solution to each well using the BIOHIT e1200 multi-dispenser pipette and BIOHIT filter tips.

9.5.12. Incubate for 30 min-1 hour at room temperature - do not stack plates.

9.5.13. Remove BCIP/NBT (12 ml per plate) into a 50 ml tube. Filter into a fresh 50 ml tube using a 50 ml syringe and 0.45 um filter and allow to warm to RT (protect from light using foil)

9.5.14. Dilute secondary antibody 1:1000 in 3% Marvel in TBS (12 µl in 12 ml per plate).

9.5.15. Wash plates 4 times with RT D-PBS (Immerse plates in TBS in a sandwich box and flick into the sink, blot on blue roll. Wash and flick a further 3 times. Finally blot on blue roll to ensure all PBS is removed).

9.5.16. Add 100 µl of 1 x Secondary antibody solution (anti-mouse AP- conjugated) to each well using the BIOHIT e1200 multidispenser pipette and BIOHIT filter tips.

9.5.17. Incubate for 30 min-1 hour at room temperature - do not stack plates.

9.5.18. Wash plates 4 times with RT D-PBS (Immerse plates in TBS in a sandwich box and flick into the sink, blot on blue roll. Wash and flick a further 3 times. Finally blot on blue roll to ensure all PBS is removed).

9.5.19. Add 100 µl of BCIP/NBT solution to each well using the BIOHIT e1200 multidispenser pipette and BIOHIT filter tips.

9.5.20. Incubate for at least 10 minutes or until indigo dots start to appear.

9.5.21. Remove BCIP/NBT by flicking in the sink and wash plate five times in tap water, after the final wash, tap plate repeatedly on blue roll to remove as much water as possible.

9.5.22. Remove the lids from the plates and place them upside down on a piece of blue roll protected from light and leave to dry overnight before counting.

### Counting

9.6.1 Using the AID Elispot reader:

a. Cut a piece of white paper or card to fit underneath the plate. Place this in the Elispot reader and put the plate on top.

b. Turn on the Elispot reader and log into the computer using standard Novell login credentials.

c. Place plate in the holder. We traditionally place dilution series 1 to the left.

d. Locate the program ‘Eli’ in the C:/Elispot4.osr folder. Create a desktop shortcut for future use. Login and open the program:
The computer brings up an error message : layout file “does not exist”. Press OK
Username = vector
Password = vector

e. New file, click OK. Tools > Count Settings > VVCF amine > OK.

f. Click the icon to open a new document

g. Tools > Stage > Load calibration.

h. Select adeno black plate

i. Tools > Stage > Calibrate Stage.

When a window opens, click the ‘A1’ button under the ‘Calibrate these wells’ heading. Using the arrows at the right of the screen, adjust the position and size of the circle so that it fits just inside well A1. Repeat for wells A12 and H12, and press OK.

j. Press the ‘Go’ icon to read and count the plate. Click on one of the well images to view it. If the pictures is not clear, adjust the camera settings using Tools > Camera settings. If it is difficult to focus with the camera, check that the plates are dry.

If the reader is marking parts of the well that do not contain a brown dot:

Tools > Count Settings > Ad Immuno > edit. Increase the intensity threshold and recount the plate.

NOTE: It is better to miss some dots than to count extra spots, as it is easy to add spots later.

k. Open each image sequentially and add spots that the reader has missed by clicking the ‘Add spots’ (target) icon and then clicking on the brown cells. Only edit wells with 50 - 200 spots. Note which dilution series is counted in each case.

NOTE: Wells should be 100% intact to be counted. A minimum of 3 wells per virus per titration should be counted. If less than this is available the titration needs to be repeated.

1. When finished, click Save As:

S:/Vector Core Facility/Image Files/Titration Images. This will create a folder containing the well images, along with a notepad document showing the final counts.

m. When finished, click the ‘Eject’ icon to remove the plate.

9.6.2 Enter counts into the ‘Immuno Counts’ tab of the Adenovirus Production Chart on S:/Virus Production Records. The spreadsheet should calculate the titre automatically, using the formula:

“average number of spots” x “serial dilution” x “dilution factor” = “x” iu/ml

e.g. if counts of 36 and 40 are obtained at the 10⁻⁷ dilution:

(36+40) = 38 x (1x10⁷) x 20 = 7.6 x 10⁹ iu/ml

NOTE: Check that the ‘Average per Well’ cell is selecting the appropriate values in its formula.

9.6.3 Input the titre into the appropriate tab of the spreadsheet (Either ‘Stock Maintenance’ or ‘New Adenoviruses’). Calculate the P to I ratio by dividing the viral particles per ml (from spec reading) by the IU/ml from titration.

P:I values above 100 are usually considered too high for AdCh63 viruses, whilst values above 50 are usually too high for AdHu5 viruses. In this case, consider repeating the hyperflask infection and purification process.

### Appendix

### Method of Cell Fixation (February 2013).

We have demonstrated that fixation with methanol (-20 C) dehydrates the TREX cells very aggressively (we looked at dropping methanol on cells in a non-amine-coated 6 wp and they shrivelled and came away from the plastic surface). Methanol fixation must be performed at -20 C as this slows down lipid removal and reduces cell destruction, for this reason, the methanol must also be removed prior to warming the plate before staining.

We have looked at fixing with 4% formaldehyde, which is much gentler on the cells, but this resulted in the staining being very diffuse around each stained cell.

During culture, water and media commonly evaporate from the wells that are closest to the perimeter of the plate, with the outer 36 and corner wells being the most affected. The result is a variation in cell growth across the plate, while any media components, such as salt, can become concentrated to the point where they are harmful to the cells. A volume loss as small as 10% can concentrate media components and metabolites enough to alter cell physiology, consequently impacting on the viability of downstream data, causing heterogeneous or biased results to occur. If the cells are unhealthy in the outer wells, they are more likely to be lost from the plate during washing.

### Points That Have Been Considered During Method Development

### Feeding Cells After 24 Hr

We tried omitting this step - when plates are placed in the Nunc square plates, no evaporation occurs and therefore the cells seem fine in 50 µl for 48 hr

### Edge Effect

We have shown that edge effect does not occur when using the Nunc Square trays to incubate the plates in at all tc stages. We have not proven that it is a problem without the plates.

To test, we titrated control virus in all wells at the same concentration. The spots were counted using the EliSpot reader. The mean and 95% CI were calculated and no wells lay outside this range.

### Number of Spots Counted

For the counts to be meaningful when using the Poisson distribution, a certain number of events need to be counted. In the case of a titration such as this, we have to be confident that we have sufficient counts but also that we are not witnessing double infection events (a single cell infected with more than one virus particle). To this end: if we assume an upper limit of 200 spots in a well containing approx 20000 cells MOI 0.01) POISSON function (from Excel)

Returns the Poisson distribution. A common application of the Poisson distribution is predicting the number of events in a specific volume eg number of virus particles per ml.

### No of Dilution Series

### Maximum Number of Spots that can be Counted Before we see Cells Infected with More than One Virus Particle

### TCID50 and Pfu/Ml

Assuming that the same cell system is used, that the virus forms plaques on those cells, and that no procedures are added which would inhibit plaque formation, 1 ml of virus stock would be expected to have about half of the number of plaque forming units (PFUs) as TCID50. This is only an estimate but is based on the rationale that the limiting dilution which would infect 50% of the cell layers challenged would often be expected to initially produce a single plaque in the cell layers which become infected. In some instances, two or more plaques might by chance form, and thus the actual number of PFUs should be determined experimentally. Mathematically, the expected PFUs would be somewhat greater than one-half the TCID50, since the negative tubes in the TCID50 represent zero plaque forming units and the positive tubes each represent one or more plaque forming units. A more precise estimate is obtained by applying the Poisson distribution. Where P(o) is the proportion of negative tubes and m is the mean number of infectious units per volume (PFU/ml), P(o) = e(-m). For any titer expressed as a TCID50, P(o) = 0.5. Thus e(-m) = 0.5 and m = -ln 0.5 which is ~ 0.7. Therefore, one could multiply the TCID50 titer (per ml) by 0.7 to predict the mean number of PFU/ml. When actually applying such calculations, remember the calculated mean will only be valid if the changes in protocol required to visualize plaques do not alter the expression of infectious virus as compared with expression under conditions employed for TCID50.

Thus as a working estimate, one can assume material with a TCID50 of 1x 105 TCID50/ml will produce 0.7 x 105 PFUs/ml.

### Example 4

Firstly a phase I/II study Clinical Trial is carried out incorporating a First in Human study in healthy adults aged 18-50 which is conducted first. Secondly a phase II component testing the vaccine in older adults and children is carried out following review of the safety data in the first groups by a local safety committee.

The protocol is summarised below, and the CTA amendment process allows the addition of further groups, increasing the size of groups, or adding further clinical trial sites in the UK as may be required depending on the spread of nCoV infections across the world.

### Intramuscular Administration Groups

Groups 1 and 2 constitute the ‘First in Human’ component. At least 5 subjects per group are vaccinated before proceeding to other groups.

| Group | Age Group | Dose | Schedule |
|---|---|---|---|
| Group 1 (n=10) | Healthy Adults aged 18-50 | 2.5x10¹⁰ vp | Single Dose |
| Group 2 (n=10) | Healthy Adults aged 18-50 | 5x10¹⁰ vp | Single Dose |
| Group 3 (n=10) | Healthy Adults aged 18-50 | 2.5x10¹⁰ vp | Two doses 4 weeks apart |
| Group 4 (n=10) | Healthy Adults aged 18-50 | 5x10¹⁰ vp | Two doses 4 weeks apart |
| Group 5 (n=10) | Older adults aged 51-65 | 5x10¹⁰ vp | Single Dose |
| Group 6 (n=10) | Older adults aged >65 | 5x10¹⁰ vp | Single Dose |
| Group 7 (n=10) | Children aged 12-15 | 2.5x10¹⁰ vp | Single Dose |
| Group 8 (n=10) | Children aged 12-15 | 5x10¹⁰ vp | Single Dose |
| Group 9 (n=10) | Children aged 5-11 | 2.5x10¹⁰ vp | Single Dose |
| Group 10 (n=10) | Children aged 5-11 | 5x10¹⁰ vp | Single Dose |
| Group 11 (n=10) | Children aged 6 months to 4 years | 1x10¹⁰ vp | Single Dose |

Aerosol Arms - with bronchoscopy follow up

| | | | |
|---|---|---|---|
| Group | Age Group | Aersol Dose | IM Dose |
| Group 12 (n=3) | Healthy Adults aged 18-50 | 1x10⁹ vp | NA |
| Group 13 (n=3) | Healthy Adults aged 18-50 | 1x10¹⁰ vp | NA |
| Group 14 (n=10) | Healthy Adults aged 18-50 | 1x10¹⁰ vp | Saline Placebo |
| Group 15 (n=10) | Healthy Adults aged 18-50 | Saline Placebo | 5x10¹⁰ |

Suitably immunology may be assessed at baseline, 7, 28, and optionally 182 days. The protocol is disclosed in detail in the following example(s).

### Progression to Licensure

The ability to conduct vaccine licensure studies depends on the spread of nCoV infections in the coming months. In Africa there is currently extremely limited ability to conduct the diagnostic test for nCoV-2019, and in some countries, limited ability to respond if the virus was to begin to spread in densely populated areas.

If the outbreak is contained, vaccine licensure will rely on efficacy testing in animal models combined with safety and immunogenicity data from phase II trials. Efficacy testing is in hand in both ferrets and NHPs. These studies can be extended to determine correlates of protection. If, like SARS, the outbreak ends within a few months, it would still be desirable to continue vaccine development to a point where these studies had been completed and a vaccine stockpile was available.

If rapid containment is not possible, phase III vaccine efficacy studies should be carried out. The phase I/II study described here supports continued planning for further phase II and III trials in many different countries. The most likely trial design would be a randomised placebo controlled trial. Currently the case fatality rate is estimated at 1-2% with the majority of deaths occurring in older adults with pre-existing health conditions, and mild disease in the majority of the population. The trial design would therefore be based on efficacy studies of influenza vaccines. There may be a seasonal effect on SARS-CoV2 circulation, as there is for other respiratory pathogens, which should be considered when planning the studies. However after repeated human to human transmission viral mutations affecting transmissibility and disease severity may be selected. Disease severity could either increase or decrease. Increased severity would require reconsideration of the ethics of a randomised placebo-controlled trial, whereas reduced severity would put the novel coronavirus into the same category as others currently circulating, for which no vaccines have ever been deemed desirable.

Plans for eventual vaccine deployment should be considered in planning further trials. This could be vaccination of some front line health care workers, or it may need to consider efficacy in the most vulnerable population (older adults with co-morbidities), or the likely super-spreaders (young children), or the whole population. Our phase I/II trial design disclosed herein already incorporates all age groups. Should there be a need to vaccinate the whole population including special populations (pregnant women, HIV +ve) the vaccine technology described herein is appropriate.

### Example 5

### Mechanism of Action

In the clinical studies, blood samples are taken to test for IgG antibodies using a validated ELISA and T cell responses using a validated ELISpot protocol at baseline and following vaccination.

Regarding the validated ELISPOT protocol, it should be noted that the actual ELISPOT protocol is a standard technique which is typically always carried out in the same manner. The specificity for the validated ELISPOT protocol comes from the peptides used. In this invention, the peptides used are derived from the SARS-CoV2 spike protein. In one embodiment, a series of overlapping peptides are synthesised beginning with the first amino acid of the spike protein. In this embodiment, 20 mer peptides are synthesised. Therefore, the first peptide comprises the amino acid sequence of amino acids 1 to 20 of the SARS-CoV2 spike protein; the second peptide synthesised comprises amino acids 11 to 30 of the SARS-CoV2 spike protein; the third peptide synthesised comprises the amino acid sequence of amino acids 21 to 40 of the SARS-CoV2 spike protein and so on. This collection of peptides may be grouped together in pools to facilitate carrying out of the ELISPOT protocol. Any suitable approach to the pooling of the peptides may be adopted by the skilled operator.

The particular ELISPOT method and results are provided below, together with a complete list of peptides used in the analysis and details of the pooling strategy used. It is to be noted that none of the other vaccine technologies in development are expected to be used as a single dose primary series. DNA, RNA and protein vacicnes are all planned as a two dose vaccination regimen with most likely 4 weeks between doses. Thus the inventor’s vaccine is effective in generating immune responses exceptionally fast appears to offer the best vaccine option to combat SARS-CoV2.

At a period of 7 days or longer following the challenge animals will be euthanised and the lungs will be examined for evidence of immunopathology. As well as intramuscular delivery for the initial groups we include aerosol vaccine delivery followed by bronchoscopy to assess immune responses in the respiratory tract, with an intramuscular vaccination/bronchoscopy for comparison.

All of these assessments follow well established protocols already in use at either Oxford or Imperial. These studies also provide opportunities to compare vaccine immunogenicity with that of other nCoV vaccines in clinical or preclinical development.

### Chemistry, Manufacturing & Control (CMC) Development

Replication-deficient adenoviral vectored vaccines are known. The adenovirus E1 gene must be supplied in trans by the cell line used for vaccine manufacture. In HEK293 cells, this gene is flanked by other sequences from adenovirus 5 which are present in the Ad5 vaccine vector, such that in rare cases a double crossover event result in the generation of replication-competent adenovirus. This is undesirable and has been solved by either the use of a different adenoviral vector such as ChAdOx1, in which the homology between the vector and the cell line is too low to allow for recombination, or the use of a cell line which expresses Ad5 E1 with no flanking sequences such as PerC6, or others developed by different companies.

A further refinement of the cell line is to include the ability to repress expression of the vaccine antigen during manufacture. The vaccine antigen is under the control of a strong mammalian promoter in order to provide high level antigen expression after vaccination. Expression of the antigen during manufacture may have a deleterious effect on vaccine yield. By preventing vaccine expression during manufacture, the yield is no longer affected by the choice of antigen and the process may be standardised. We have access to such a cGMP cell bank for this project, and it has been used previously by both Advent (phase I/II material) and CanSino (scale-up).

The upstream process consists of expanding the cell bank, infecting with the seed virus and allowing the adenovirus to replicate within the cells. After harvest, detergent lysis, clarification and further downstream purification is achieved by standard methods which are already in place at both Advent and CanSino. The purified Drug Substance is then diluted into formulation buffer, filter sterilised and filled into vials which may be stored as liquid or lyophilised.

Quality control tests include concentration (which is the potency assay), sterility, DNA sequence of vaccine antigen and absence of adventitious agents. The use of deep sequencing has recently greatly accelerated characterisation of vaccine seed stocks, to confirm clonality without lengthy rounds of virus cloning, and also in detection of adventitious agents. Thus the time taken for release testing may be greatly shortened.

In order to initiate cGMP manufacture, the Clinical Biomanufacturing Facility at Oxford is producing a vaccine seed stock which will be suitable to transfer into a clean room for manufacturing. This will be tested for concentration, antigen DNA sequence, sterility and mycoplasma prior to being used by two vaccine manufacturers. Advent, in Italy, will produce a batch of 1000 vials for the first clinical studies. CanSino, in China, will also manufacture the vaccine. CanSino will commence manufacturing upon provision of the seed stock, starting with a 200 L batch to produce 20,000 doses.

All of the clinical trials that have been conducted with ChAdOx1 vectored vaccines have used GMP Drug Product. For ChAdOx1 SARS-CoV2, the initial GMP batch of 1000 doses provides sufficient to conduct the phase I/II clinical trial that is disclosed above. At the same time as transferring vaccine seed stock to Advent in order to manufacture the first 1000 doses, vaccine seed stock is transferred to CanSino to allow scale up manufacture.

### Summary/Timeline

ChAdOx1 SARS-CoV2 vaccine for preclinical studies generated
   Finished Feb 18th 2020
Preclinical testing including initial immunogenicity studies in mice followed by efficacy studies in ferrets and NHPs
   Implemented under agreement at NIH and CSIRO
Vaccine seed stock suitable for cGMP manufacture generated
   Finished March 6th 2020
Phase I/II batch of 1000 doses vaccine manufacture to cGMP and made available for clinical trials
   In progress
Obtain ethical and regulatory approval for a UK trial
   In progress
Conduct Phase I/II study, providing safety and immunogenicity data in adults, older adults and children
   In progress
Large scale manufacture of vaccine (200 L, estimated 20,000 doses per batch at first)
   In progress (Seed stock transferring)

### Example 6

These initial studies test immunogenicity of a single vaccine dose. Both B and T cell responses are assessed after two weeks. IgG responses are assessed with an ELISA assay using protein produced by Keith Chapell (UQ Australia). Neutralising antibody is measured using a pseudotyped virus carrying the nCoV Spike protein on the surface. This assay has been used to verify verified the 2019-nCoV entry receptor (https://www.biorxiv.org/content/10.1101/2020.01.22.915660v1). T cell responses are measured in an ELISpot assay using peptides covering the entire Spike protein sequence.

Vaccine is provided to Rocky Mountain labs, NIH, for non-human primate vaccination and efficacy studies. One group receives a control vaccination with ChAdOx1 green fluorescent protein (GFP), one with a single dose of ChAdOx1 SARS-CoV2 and one with two doses administered four weeks apart, with virus challenge after a further four weeks. A thorough histopathology study takes place following the challenge study to assess any possible immunopathology. The inventors assert that NHPs are protected by vaccination with no viral replication after challenge, and no evidence of immunopathology.

A further vaccine efficacy study takes place in ferrets, conducted by PHE, or by CSIRO if capacity is limited at PHE. This consists of the same groups as in the NHP study but with a fourth group vaccinated with ChAdOx1 nCoV at two timepoints from which animals are removed at different times after challenge to assess immunopathology in the lungs.

The pre-GMP vaccine seed stock is produced at the Clinical Biomanufacturing Facility, Oxford. This is transferred to Advent for preparation of a Master Virus Bank and Drug Substance. The first vaccine fill and finish results in 1000 vials being produced, with potential for more in a second fill. Vaccine quality testing is in hand with the MHRA with employing deep sequencing methods to reduce the time taken for certification to GMP.

The clinical study commences with a dose escaltion in healthy adult volunteers between the ages of 18 and 50. The standard approach for First in Human studies is to intially vaccinate with the lowest dose in a single volunteer. Following successful safety review, the same dose is administered to two other volunteers, with the remainder of the group then vaccinated forty-eight hours later after a further safety review. The first dose will be 2.5x 10¹⁰ vp, which the inventors assert is immunogenic with no SAE. If a higher dose of 5x 10¹⁰ vp induces limited and short-lived fevers in some subjects then the lower dose can be selected, or adjusted accordingly. Thus these two doses are tested and one dose selected for further clinical assessment.

Following safety review of the first two groups after one week post vaccination, the study will continue into adults over 50, and then into school age children. For the adult groups, we recruit, vaccinate and perform follow up immunogenicity and safety assessment of vaccinated volunteers.

Safety review will follow standard procedures with visits for assessment at 2,7,14, 28, 56, and 182 days after vaccination. Immunogenicity is assessed on day of vaccination plus days 7, 14, 28, 56 and 182 in adults with a maximum of three timepoints for children. Immunogenicity assessments include ELISA and ELISpot assays as the primary immunology endpoints. In addition neutralisation assays on live coronavirus and T cell phenotyping are conducted. PBMCs are frozen and may be used for further immunology studies investigating the breadth of response, or for preparation of monoclonal antibodies.

The studies described here represent the best practice for vaccine development against novel coronavirus, and are conducted to GCP as fast as possible. Clinical studies are followed by age escalation and de-escalation studies. The age groups to be included allow assessment of potential vaccine performance in healthcare workers, older adults at risk of more severe disease, and children who may experience mild disease but transmit the infection very effectively to others. Following these initial studies, more detailed immunology assessments continue, as well as clinical vaccine efficacy studies.

### Example 7 Growth Curve of ChAdOx1-2019nCoV

HEK293 TREx suspension cells were cultured in the following media:

| | |
|---|---|
| Constituent | Supplier |
| 1 L CD293 Media | Fisher 11913019 |
| 5 ml FBS | Sigma F2442 |
| 1 ml 100x Pen / Strep | Sigma P0781 |
| 20 ml 200 mM Glutamine | Sigma G7513 |
| 100 µl 10 mg/ml Blasticidin | Melford Labs B1105 |
| 10 ml anti-clumping agent | Fisher 0010057DG |
| 20 ml 1 M HEPES | Sigma H0887 |

HEK 293 TREx cells express the tetracycline repressor protein which binds to sites in the CMV promoter of the recombinant adenovirus and prevent expression of the nCoV-19 spike protein during production of the ChAOx1 nCoV-19 in these cells. Expresssion of the tet repressor protein is switched off when tetracycline is added to the culture medium, allowing the nCoV-19 spike protein to be expressed.

The day prior to infection, HEK293 TREx cells were pelleted and re-suspended in minimal media (CD293, 1% FBS, 5 mM L-Glutamine and pen / strep), counted by trypan blue exclusion and seeded at 1x10e6 /ml. The culture flask was left to grow overnight (37° C., 5% CO₂, within an orbital incubator).

On the day of infection the cells were counted by trypan blue exclusion and adjusted to 1x10e6 /ml with minimal media. Cells were aliquoted into 80 ml volumes in fresh culture flasks and various additions made to each flask:
Flask 1: Repressed MOI 3: 8 µl Blasticidin + virus at a multiplicity of infection (MOI) of 3
Flask 2: de-repressed MOI 3: 80 µl of 1 mg/ml tetracycline + virus MOI 3
Flask 3: Repressed MOI 1: 8 µl Blasticidin + virus MOI 1
Flask 4: Repressed MOI 0.3: 8 µl Blasticidin + virus MOI 0.3
Flask were returned to incubate (37° C., 5% CO₂)
From uninfected cells, a 500 µl volume was taken and pelleted. The pellet and supernatant were stored at -80° C. separately to be used as a negative control in qPCR.

At 24, 48 and 72hpi the following samples were taken:

A- 500µl: pelleted by centrifugation and both supernatant and pellet stored separately at -80° C. to be analysed by qPCR.

B- 2ml: pelleted by centrifugation. Supernatant was recovered and placed into a separate tube. The cell pellet was first re-suspended in 140 µl of ChAdOx1 lysis buffer containing nuclease. The total volume was then made up to 200 µl using 5 M NaCl. Sample was vortexed. Both cell pellet and supernatant samples were placed at -80° C. to be used in immune-titration assays to calculate the IU.

### C- Quantification of Infectious Units (IU)

IU was quantified using a titre immunoassay. Briefly, a black walled / clear flat bottomed 96 well plate (Corning) was seeded with adherent HEK293 TREx cells in standard growth media (below) to obtain a 95% confluent monolayer on the day required.

| | |
|---|---|
| Constituent | Supplier |
| 500 ml 1x DMEM | Sigma D6546 or Gibco 21969035 |
| 50 ml FBS | Sigma F2442 |
| 5 ml 100x Pen / Strep | Sigma P0781 |
| 10 ml 200 mM Glutamine | Sigma G7513 |
| 250 µl 10 mg/ml Blasticidin | Melford Labs B1105 |
| 10 ml 1 M HEPES | Sigma H0887 |

Samples to titrate were thawed, vortexed and a 10 µl aliquot taken to test. This was mixed with 90 µl growth media to produce a 10⁻¹ dilution. Further dilutions in standard growth media (10⁻² to 1.1x10⁻⁷) were made in duplicate per sample across an empty V-bottomed 96 well plate. Media from the assay plate was removed and 50 µl of each test sample / dilution was plated. Plates were incubated for 24 h (37° C., 8% CO₂) before a further 50 µl standard growth media was added. Plate was returned to the incubator for a further 24 h. After a total of 48 h, all well contents were aspirated and the cells fixed with 100 µl per well pre-chilled Methanol. Plates were placed at -20° C. overnight.

To immunostain all incubation steps were performed at room temperature: plates were washed (x5) with PBS before blocking first with 100 µl per well Bloxall (Vector Labs) for 30 mins and then 200 µl per well 1% casein solution (Thermo Fisher) for 15 mins after washing (x5) with PBS. Anti-adenoviral hexon antibody (AbCam) diluted in 1% casein solution was added to wells (100 µl / well). After 1 h the primary antibody was removed and plates washed (x8) with 1x TBS (Tris Buffered Saline - Sigma). Secondary antibody (goat anti-mouse IgG whole molecule, Sigma) was diluted in TBS containing 3% skim milk powder. This was added 100 µl / well before a further 1h incubation. Plates were again washed (x8) in 1xTBS before 100 µl per well BCIP / NBT was added per well to visualise infected cells. Once ‘spots’ had stained well, BCIP / NBT was removed, plate washed (x5) in tap water and left to dry overnight. Images were obtained of each well using the AID Elispot reader and distinct spots counted in wells where 20-200 could be seen. The IU titre was assessed by calculating the dilution factor of each given sample and the number of spots counted at that dilution.

### Results

FIG. 1: Total IU within an 80 ml culture infected at MOI 3 with and without repression Repressed and de-repressed cultures gave a similar IU of virus at all time points tested.

FIG. 2: total IU decreases in a dose dependent manner according to MOI

### Quantification of Genome Copy Number within Cultures

Samples were taken from storage at -80° C. and thawed at room temperature. Pellet samples were re-suspended in 500 µl molecular grade water to return them to their previous concentration volume in culture.

All samples were diluted 10 µl in 15 µl DNArealeasy (Anachem) and the following PCR programme used to generate viral DNA template:

65° C. for 15 mins, 96° C. for 2 mins, 65° C. for 4 mins, 96° C. for 1 min, 65° C. for 1 min, 96° C. for 30 secs.

For a standard curve ChAdOx1 plasmid DNA of a known concentration was diluted to generate sample of a given copy number per well. qPCR master mix was prepared using 2x Luna probe mix (NEB), ChAdOx2 specific primers (Thermo Fisher), ChAdOx1 specific universal probe (TAMRA / FAM) (Applied Biosystems) and nuclease free water to a final volume of 15 µl per sample. Mastermix was mixed and 15 µl added to the relevant wells of a 96 well MicroAmp FAST Optical PCR plate. Template / plasmid standard / samples were added (5 µl per well) to relevant test wells. Optical film was used to cover the plate before the relevant qPCR programme was run on a StepOne qPCR machine.

PCR programme: 95° C. for 10 mins, 45 cycle of 95° C. for 15 sec, 60° C. for 1 min. Recovered data was analysed using the standard curve results to generate viral genome copy number per well, which was further calculated to give genome copy per ml culture.

To compare the IU titre between de-repressed and repressed, the genome copy number values of the de-repressed culture were set at 100% and the graph below shows the difference of the repressed culture compared to this.

### Results

FIG. 3: Genome copy number within flasks depicted as percentage standardising 100% as output from de-repressed culture.

The data indicate that genome copy number is similar in both conditions under test.

### Example 8

### A Phase ⅔ Study to Assess the Efficacy and Safety of a Recombinant Adenovirus-Based Vaccine Against Coronavirus Disease (COVID-19)

A single-blind, randomized safety and efficacy study, with immunogenicity sub studies in older and younger age groups

Main efficacy trial: Healthy adults aged ≥18 years.

Sequential age escalation/de-escalation immunogenicity sub studies:
1. Healthy adults aged 56 - 70 years, inclusive
2. Healthy adults aged 71 years or older
3. Healthy children aged 5 to 15 years, inclusive

Total number to enrol: 3,000 - 5,250 participants
Sequential age escalation/de-escalation groups:
Group 1 adults aged 56 - 70 years:
ChAdOx1-nCOV19 5x1010vp, N=30
OR
Placebo, injectable Saline, N=30
Group 2 adults aged 71+ years:
ChAdOx1-nCOV19 5x1010vp, N=50
OR
Placebo, injectable Saline, N=50
Group 3 children aged 5 - 15 years:
ChAdOx1-nCOV19 2.5x1010 vp, N=30
OR
Placebo, injectable Saline, N=30
Main efficacy study: 5280 participants
Group 4 adults aged ≥18 years:
ChAdOx1-nCOV19 5x1010 vp, N= up to 2500
OR
Placebo, injectable Saline, N= up to 2500

Planned Trial Duration 6 - 12 months per participant

| | | |
|---|---|---|
| | Objective | Outcome Measure |
| Primary | To investigate safety and efficacy of ChAdOx1-nCOV19 vaccine in healthy adults aged ≥18 years of age | Safety: 1. local and systemic adverse reactions for 7 days post-vaccination in a subset of 100 participants. |
| | | 2. Serious Adverse Events Efficacy: Virologically confirmed (PCR positive) symptomatic cases of COVID-19 |
| Secondary | To assess cellular and humoral immunogenicity of ChAdOx1 nCoV-19 | a) Interferon-gamma (IFN-y) enzyme-linked immunospot |
| | | (ELISpot) responses to SARS-CoV-19 2 spike protein; |
| | | b) Enzyme-linked immunosorbent assay (ELISA) to quantify antibodies against SARS-CoV-2 spike protein (seroconversion rates); |
| | | c) virus neutralising antibody (NAb) assays against live and/or pseudotype SARS-CoV-2 virus |
| | To investigate safety and immunogenicity of ChAdOx1-nCOV19 vaccine in healthy adults aged 56-70 years, and 71 years or older | Antibodies against SARS-CoV-2 spike protein measured by ELISA at Day 28 post-vaccination. Proportion with 4-fold rise in antibodies against SARS-CoV-2 spike protein measured by ELISA at Day 28 post-vaccination. |
| | To investigate safety and immunogenicity of ChAdOx1-nCOV19 vaccine in healthy children aged 5-15 years | Antibodies against SARS-CoV-2 spike protein measured by ELISA at Day 28 post-vaccination. Proportion with 4-fold rise in antibodies against SARS-CoV-2 spike protein measured by ELISA at Day 28 post-vaccination. |
| | To investigate impact ChAdOx1- nCOV19 vaccine on serious COVID19 infections | 1. Hospitalisation for COVID19 infection |
| | | 2. ICU admission for COVID19 infection |
| | | 3. Death from COVID19 infection |
| Tertiary | Exploratory Immunology | a) Cell analysis by flow cytometry assays |
| | | b) Functional antibody assays |
| | | c) Others |
| | Measure exposure to COVID-19 | Reported by weekly survey to collect information about cases amongst household contacts and friends, contact with the general public, infection control procedures |
| Investigational products | ChAdOx1-nCoV-19, a replication-deficient simian adenoviral vector expressing the spike (S) protein of SARS-CoV-2 Sodium Chloride 0.9% solution for injection | |
| Formulation | Liquid | |
| Route of Administration | Intramuscular (IM) | |
| Dose per Administration | Group 1, 2 and 4: 5 x 10¹⁰ VP | |
| | Group 3: 2.5 x 10¹⁰ VP | |

### Example 9

Have one group of three female BALB/c and one group of five female CD-1 mice aged 6-10 weeks. Have one group of two female BALB/c and one group of three female CD-1 mice aged 6-10 weeks. Each mouse was injected intramuscularly with the requisite volume of vaccine. For intramuscular route vaccinations: injections are performed by administering 50 uL into the thigh. After 9 days the BALB/c mice were culled, after ten days the CD-1 mice were culled. The spleens were harvested of these mice and an ELIspot assay performed as detailed below and described elsewhere (PMID: 23485942).

ELISpot plate were coated with 50µL per well of coating mAb (e.g. AN18 anti-mouse IFN-γ diluted to 5 µg/mL in coating buffer). A single cell suspension from the spleen is prepared by mechanical crushing, lyisis and differential centrifugation as described elsewhere (PMID: 23485942). Splenocytes were incubated with peptides (1-4ug/ml) spanning the whole spike protein encoded in the ChAdOx1 nCoV-19 vaccine. Peptide 1 had the sequence MFVFLVLLPLVSSQC (SEQ ID NO: 16); peptide 2 had the sequence LVLLPLVSSQCVNLT (SEQ ID NO: 17); peptide 3 had the sequence PLVSSQCVNLTTRTQ (SEQ ID NO: 18) and so on up to and including peptide 316. Peptides 317 to 321 were overlapping 15mers in the same manner, but having the sequence from tPA. ELISpot plates were developed and analysed, data is presented below.

Pool 1: peptides 1-77 inclusive; 317-321 inclusive. Pool 2: Peptides 78 to 167 inclusive. Pool 3: Peptides 168 to 241 inclusive. Pool 4: Peptides 242 to 316 inclusive.

FIG. 4. Summed splenic IFN-γ ELISpot responses of BALB/c (left panel) and CD-1 (right panel) mice, in response to peptides spanning the spike protein from SARS-CoV-2, nine or ten days post vaccination, with 1.7 × 10¹⁰ vp ChAdOx1 nCoV-19 or 8 × 10⁹ vp ChAdOx1 GFP. Mean with SEM are depicted.

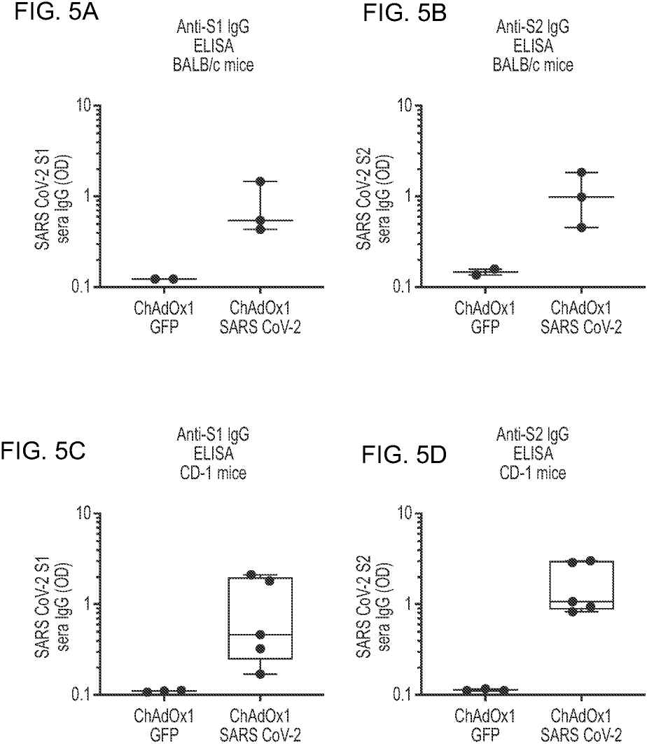

FIG. 5. Box and whisker plot of the optical densities following ELISA analysis of BALB/C mouse sera (Top panel) incubated with purified protein spanning the S1 domain (left) or purified protein spanning the S2 domain (right) of the SARS-CoV-2 spike nine or ten days post vaccination, with 1.7 × 10¹⁰ vp ChAdOx1 nCoV-19 or 8 × 10⁹ vp ChAdOx1 GFP. Box and whisker plots of the optical densities following ELISA analysis of CD-1 mouse sera (Bottom panel) incubated with purified protein spanning the S1 domain (left) or purified protein spanning the S2 domain (right) of the SARS-CoV-2 spike.

### Example 10 : Assembly of Vaccine

### Physical, Chemical and Pharmaceutical Properties and Formulation

### Description of ChAdOxl nCoV-19

ChAdOx1 nCoV-19 vaccine consists of the replication-deficient simian adenovirus vector ChAdOx1, containing the structural surface glycoprotein (Spike protein) antigen of the SARS CoV-2 (nCoV-19) expressed under the control of the CMV promoter, with a leading tissue plasminogen activator (tPA) signal sequence. The tPA leader sequence has been shown to be beneficial in enhancing immunogenicity.

The code name for the Drug Substance is ChAdOx1 nCoV-19. There is no recommended International Non-proprietary Name (INN).

The ChAdOx1 nCoV-19 drug substance has a genome size of 35,542 bp and is a slightly opaque frozen liquid, essentially free from visible particulates. The appearance is dependent upon the concentration of the virus and the buffer that the virus is formulated in.

### ChAdOxl Vector

The ChAdOx1 vector is replication-deficient as the E1 gene region, essential for viral replication, has been deleted. This means the virus will not replicate in cells within the human body. The E3 locus is additionally deleted in the ChAdOx1 vector. ChAdOx1 propagates only in cells expressing E1, such as HEK293 cells and their derivatives or similar cell lines such as Per.C6 (Crucell).

### ChAdOxl nCoV-19 Vaccine Strain Assembly

The vaccine consists of the attenuated chimpanzee adenovirus vector ChAdOx1, expressing the SARS CoV-2 spike protein under the control of the CMV promoter. Pre-adenoviral plasmid pBAC ChAdOx1 nCoV19 was generated and prepared at the Jenner Institute, University of Oxford. The SARS CoV-2 Spike cDNA including a 32 amino acid N-terminal tPA leader sequence, obtained from GeneArt, was inserted into the E1 locus of ChAdOx1 by Gateway recombination. Suitably the “long CMV promoter” is used. This is known in the art, and is described in PCT/GB2008/001262 (WO/2008/122811).

The following DNA constructs were used:
#p5727: SARS CoV-2 Spike cDNA in DNA vector pMK
#p1990: pENTR plasmid vector containing the CMV ‘long’ promoter (with intron A and Tet operator sites; CMVLP TO) and the BGH poly A sequence.
#p5710: pENTR plasmid vector containing the CoV Spike antigen between the ‘long’ CMVLP TO promoter and BGH poly A sequences.
#p2563: pBAC ChAdOx1 vector with E1 and E3 deleted, and E4 modified to improve yield and hexon expression for markerless titration. It was generated at the Jenner Institute, and its complete genome sequence is known

The SARS CoV-2 Spike antigen was excised from #p5727 using NotI and KpnI and ligated into #1990 cut with the same enzymes to obtain #p5710. The insert was verified by restriction mapping and sequencing. Gateway recombination was then performed between #5710 and #2563.

The sequence of the transgene region in ChAdOx1 nCoV-19 has been verified by sequencing directly from phenol purified viral genomic DNA.

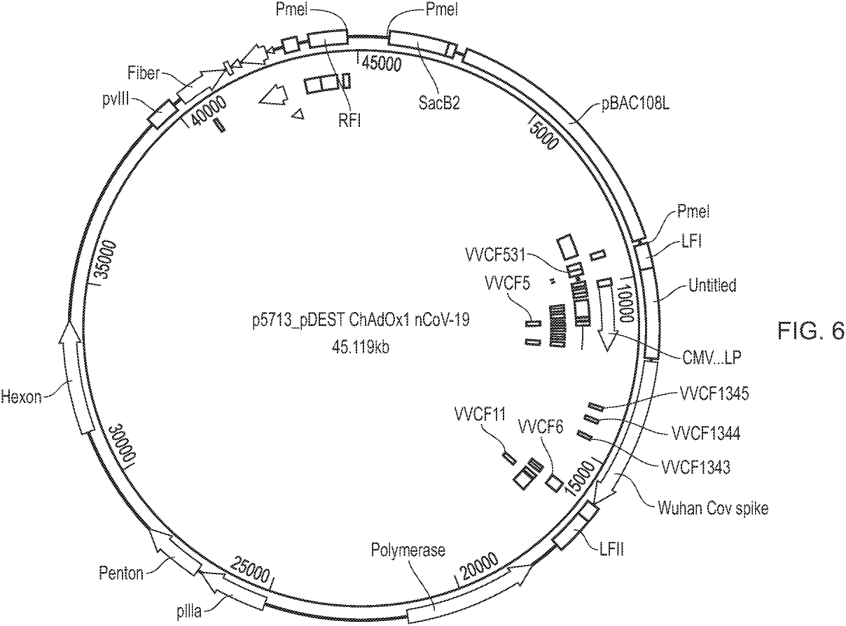

The DNA map of #p5713 pBAC ChAdOx1 nCoV-19 used to generate the recombinant viral vector vaccine is shown in FIG. 6.

In more detail, the p5713 pDEST-ChAdOx1-nCOV-19 plasmid is used in the manufacture of the composition according to the present invention. Specifically, the plasmid encodes a viral vector according to the invention. The viral sequence is excised from p5713 pDEST-ChAdOx1-nCOV-19 and the linear viral DNA is subsequently used to transfect E1 expressing cells, such as HEK293-TRex cells, for viral vaccine production.

SEQ ID NO: 25 - p5713 pDEST-ChAdOx1 nCoV-19 DNA Sequence. Format: DNA (top strand), 44104 nucleotides.

### Notable Features

“Long” CMV promoter (CMVLP) containing intron A, and Tet operator (TO) sites for repression of transgene expression in cells expressing the Tet repressor
Synthetic codon-optimised SARS CoV-2 spike protein open reading frame
BGH polyA signal
Flanking site-specific recombination sequences utilised for transgene insertion.
Chloramphenicol resistance gene in BAC vector backbone
PmeI sites for release of viral genome

### Example 11

Trial Title: A phase I/II study to determine efficacy, safety and immunogenicity of the candidate Coronavirus Disease (COVID-19) vaccine ChAdOx1 nCoV-19 in UK healthy adult volunteers
Study Reference: COV001
Protocol Version: 0.5
Date: 12 MAR 2020
EudraCT number: 2020-001072-15
IRAS Reference: 281259

| Design | Single-blinded, randomised, placebo controlled, multi-centre | | |
|---|---|---|---|
| Population | Healthy adults aged 18-55 years | | |
| Planned Sample Size | Up to 510 | | |
| | Group | W0 | W4 |
| | 1a (n=44) | ChAdOx1 nCoV-19 | - |
| | Intense Follow-up | 5x10¹⁰ vp | |
| | 1b (n=44) Intense Follow-up | Saline Placebo | |
| | 2a (n=206) | ChAdOx1 nCoV-19 5x10¹⁰ vp | - |
| | 2b (n=206) | Saline Placebo | - |
| | 3 (n=10) Prime-boost | ChAdOx1 nCoV-19 5x10¹⁰ vp | ChAdOx1 nCoV-19 5x10¹⁰ vp |
| Planned Duration | Trial | 6 months from enrolment with a 12 months optional visit | |
| | Objective | | Outcome Measure |
| Primary | To assess efficacy of the candidate ChAdOx1 nCoV-19 against COVID-19 | | a) Virologically confirmed (PCR positive) symptomatic cases of COVID-19 |
| Co-Primary | To assess the safety of the candidate vaccine ChAdOx1 nCoV | | a) occurrence of serious adverse events (SAEs) throughout the study duration. |
| Secondary | To assess the safety, tolerability and reactogenicity profile of the candidate vaccine ChAdOx1 nCoV | | a) occurrence of solicited local reactogenicity signs and symptoms for 7 days following vaccination; b) occurrence of solicited systemic reactogenicity signs and symptoms for 7 days following vaccination; c) occurrence of unsolicited adverse events (AEs) for 28 days following vaccination; d) change from baseline for safety laboratory measures and; e) Occurrence of disease enhancement episodes |
| | To assess efficacy of the candidate ChAdOx1 nCoV-19 against severe and non-severe COVID-19 | | a) Hospital admissions associated with COVID-19 b) Intensive care unit (ICU) admissions associated with COVID-19 c) Deaths associated with COVID-19 d) Seroconversion against non-Spike SARS-CoV-2 antigens |
| | To assess cellular and humoral immunogenicity of ChAdOx1 nCoV-19 | | a) Interferon-gamma (IFN-y) enzyme-linked immunospot (ELISpot) responses to SARS- b) Enzyme-linked immunosorbent assay (ELISA) to quantify antibodies against SARS-CoV-2 spike protein (seroconversion rates);CoV-2 spike protein; c) virus neutralising antibody (NAb) assays against live and/or pseudotype SARS-CoV-2 virus |
| Exploratory | Exploratory Immunology | | a) Cell analysis by flow cytometry assays b) Functional antibody assays c) Others |
| Sample analysis for the completion of exploratory endpoints may be performed under the OVC Biobankf resurch tissue bank protocol (REC: 16/SC/0141). | | | |
| Investigational products | a) ChAdOx1 nCoV-19, a replication-deficient simian adenoviral vector expressing the spike (S) protein of SARS-CoV-2 b) Sodium Chloride 0.9% solutionfor injection | | |
| Formulation | Liquid | | |
| Route of Administration | Intramuscularly (IM) into the deltoid region of the arm | | |
| | ChAdOx1 nCoV-19 5x10¹⁰ vp | | |
| Dose per Administration | Saline solution for injection at same volume required in investigational arms | | |

ABBREVIATIONS

| | |
|---|---|
| AdHu | Human adenovirus |
| AdHu5 | Human adenovirus serotype 5 |
| AE | Adverse event |
| AID | Autoimmune Disease |
| CCVTM | Centre for Clinical Vaccinology and Tropical Medicine, Oxford |
| CBF | Clinical Bio manufacturing Facility |
| CEF | Chick embryo fibroblast |
| ChAd63 | Chimpanzee adenovirus 63 |
| CI | Confidence interval |
| COP | Code of Practice |
| CRF | Case Report Form or Clinical Research Facility |
| CS or CSP | Circumsporozoite protein |
| CTRG | Clinical Trials & Research Governance Office, Oxford University |
| CTL | Cytotoxic T Lymphocyte |
| DSUR | Development Safety Update Report |
| ELISPOT | Enzyme-linked immunospot |
| GCP | Good Clinical Practice |
| GMO | Genetically modified organism |
| GMT | Geometric Mean Titre |
| GP | General Practitioner |
| GSK | GlaxoSmithKline |
| HCG | Human Chorionic Gonadotrophin |
| HBV | Hepatitis B virus |
| HEK | Human embryonic kidney |
| HCV | Hepatitis C virus |
| HIV | Human immunodeficiency virus |
| HLA | Human leukocyte antigen |
| HRA | Health Research Authority |
| HTLV | Human T-Lymphotrophic Virus |
| IB | Investigator Brochure |
| ICH | International Conference on Harmonisation |
| ICMJE | International Committee of Medical Journal Editors |
| ICS | Intracellular Cytokine Staining |
| IDT | Impfstoffwerk Dessau-Tornau Biologika GmbH |
| ID | Intradermal |
| IFNγ | Interferon gamma |
| IM | Intramuscular |
| IMP | Investigational Medicinal Product |
| IMP-D | Investigational Medicinal Product Dossier |
| IV | Intravenous |
| LSOC | Local safety oversight clinician |
| ME-TRAP | Multiple epitopes and thrombospondin related adhesion protein |
| MHRA | Medicines and Healthcare Products Regulatory Agency |
| MVA | Modified vaccinia virus Ankara |
| NANP | N-acetylneuraminic acid phosphatase |
| NHS | National Health Service |
| NIH | National Institutes of Health |
| NIHR | National Institute for Health Research |
| PBMC | Peripheral blood mononuclear cell |
| Pb | Plasmodium Berghei |
| PCR | Polymerase chain reaction |
| PI | Principal Investigator |
| pfu | Plaque forming unit |
| QP | Qualified Person |
| qPCR | Quantitative polymerase chain reaction |
| QS21 | Quillaja saponaria saponin molecule |
| REC | Research Ethics Committee |
| SAE | Serious adverse event |
| SC | Subcutaneous |
| SmPc | Summary of Product characteristics |
| SOP | Standard Operating Procedure |
| SUSAR | Suspected unexpected serious adverse reaction |
| µg | microgram |
| vp | viral particle |
| VV | viral vector |
| WHO | World Health Organisation |

### 3.1 Background

A novel coronavirus, known as 2019-nCoV [1] was subsequently renamed to SARS-CoV-2 because it is similar to the coronavirus responsible for severe acute respiratory syndrome (SARS-CoV), a lineage B betacoronavirus. SARS-CoV-2 belongs to the phylogenetic lineage B of the genus Betacoronavirus and it recognises the angiotensin-converting enzyme 2 (ACE2) as the entry receptor [4].

The spike protein is a type I, trimeric, transmembrane glycoprotein located at the surface of the viral envelope of CoVs, which can be divided into two functional subunits: the N-terminal S1 and the C-terminal S2. S1 and S2 are responsible for cellular receptor binding via the receptor binding domain (RBD) and fusion of virus and cell membranes respectively, thereby mediating the entry of SARS-CoV-2 into target cells. [3] The roles of S in receptor binding and membrane fusion make it an ideal target for vaccine and antiviral development, as it is the main target for neutralising antibodies.

ChAdOx1 nCoV-19 vaccine consists of the replication-deficient simian adenovirus vector ChAdOx1, containing the structural surface glycoprotein (Spike protein) antigen of the SARS CoV-2 (nCoV-19), with a leading tissue plasminogen activator (tPA) signal sequence. ChAdOx1 nCoV-19 expresses a codon-optimised coding sequence for the Spike protein from genome sequence accession GenBank: MN908947. The tPA leader sequence has been shown to be beneficial in enhancing immunogenicity of another ChAdOx1 vectored CoV vaccine (ChAdOx1 MERS) [5].

### 3.2.1 Immunogenicity

Mice (balb/c and CD-1) were immunised with ChAdOx1 expressing SARS-CoV-2 Spike protein or green fluorescent protein (GFP). Spleens were harvested for assessment of IFN-γ ELISpot responses and serum samples were taken for assessments of S1 and S2 antibody responses on ELISA at 9 or 10 days post vaccination. The results of this study show that a single dose of ChAdOx1 nCoV was immunogenic in mice.

FIG. 4. Summed splenic IFN-γ ELISpot responses of BALB/c (left panel) and CD-1 (right panel) mice, in response to peptides spanning the spike protein from SARS-CoV-2, nine or ten days post vaccination, with 1.7 × 10¹⁰ vp ChAdOx1 nCoV-19 or 8 × 10⁹ vp ChAdOx1 GFP. Mean with SEM are depicted

FIG. 5. Box and whisker plot of the optical densities following ELISA analysis of BALB/C mouse sera (Top panel) incubated with purified protein spanning the S1 domain (left) or purified protein spanning the S2 domain (right) of the SARS-CoV-2 spike nine or ten days post vaccination, with 1.7 × 10¹⁰ vp ChAdOx1 nCoV-19 or

8 × 10⁹ vp ChAdOx1 GFP. Box and whisker plots of the optical densities following ELISA analysis of CD-1 mouse sera (Bottom panel) incubated with purified protein spanning the S1 domain (left) or purified protein spanning the S2 domain (right) of the SARS-CoV-2 spike.

### 3.2.2 Efficacy

Pre-clinical efficacy studies of ChAdOx1 nCoV-19 in ferrets and non-human primates are underway.

### 1.2 Rationale

The COVID-19 epidemic has caused major disruption to healthcare systems with significant socioeconomic impacts. Containment measures have failed to stop the spread of virus, which is fast approaching pandemic levels. There are currently no specific treatments available against COVID-19 and accelerated vaccine development is urgently needed.

Live attenuated viruses have historically been among the most immunogenic platforms available, as they have the capacity to present multiple antigens across the viral life cycle in their native conformations. However, manufacturing live-attenuated viruses requires complex containment and biosafety measures. Furthermore, live-attenuated viruses carry the risks of inadequate attenuation causing disseminated disease, particularly in immunocompromised hosts. Given that severe disease and fatal COVID-19 disproportionally affect older adults with co-morbidities, making a live-attenuated virus vaccine is a less viable option. Replication competent viral vectors could pose a similar threat for disseminated disease in the immuno-suppressed. Replication deficient vectors, however, avoid that risk while maintaining the advantages of native antigen presentation, elicitation of T cell immunity and the ability to express multiple antigens [9]. Subunit vaccines usually require the use of adjuvants and whilst DNA and RNA vaccines can offer manufacturing advantages, they are often poorly immunogenic requiring multiple doses, which is highly undesirable in the context of a pandemic.

Chimpanzee adenovirus vaccine vectors have been safely administered to thousands of people using a wide range of infectious disease targets. ChAdOx1 vectored vaccines have been given to over 320 volunteers with no safety concerns and have been shown to be highly immunogenic at single dose administration. Of relevance, a single dose of a ChAdOx1 vectored vaccine expressing full-length spike protein from another betacoronavirus (MERS-CoV) has shown to induce neutralising antibodies in recent clinical trials.

Data generated in this study will be used to support further larger phase II/III efficacy studies, which will include target groups at higher risk of severe disease.

4. OBJECTIVES AND ENDPOINTS

| | | |
|---|---|---|
| Objectives | Outcome Measures | Time point(s) of evaluation of this outcome measure |
| Primary Objective To assess efficacy of ChAdOx1 nCoV-19 against COVID-19 | a) Virologically confirmed (PCR positive) symptomatic cases of COVID-19 | Throughout the study |
| Co-primary Objective To assess the safety of the candidate vaccine ChAdOx1 nCoV | a) occurrence of serious adverse events (SAEs) throughout the study duration | Throughout the study |
| Secondary Objectives To assess the safety, tolerability and reactogenicity profile of the | a) occurrence of solicited local reactogenicity signs and symptoms for 7 days following vaccination | Day 0-6 Self-reported symptoms recorded using electronic diaries |
| candidate vaccine ChAdOx1 nCoV | b) occurrence of solicited systemic reactogenicity signs and symptoms for 7 days following vaccination; | Day 0-6 Self-reported symptoms recorded using electronic diaries |
| | c) occurrence of unsolicited adverse events (AEs) for 28 days following vaccination; | Day 0-27 Self-reported symptoms recorded using electronic diaries |
| | d) change from baseline for safety laboratory measures and; | Blood samples drawn at enrolment (before vaccination), day 2, 7 and 28 |
| | f) Occurrence of SAE of special interest: enhancement episodes | Throughout the study |
| To assess efficacy of ChAdOx1 nCoV-19 against COVID-19 | a) Hospital admissions associated with COVID-19 | Throughout the study |
| | b) Intensive care unit (ICU) admissions associated with COVID-19 | Throughout the study |
| | c) Deaths associated with COVID-19 | Throughout the study |
| | d) Seroconversion against non-Spike antigens measured by ELISA | Blood samples drawn at D0, D28 and D182 (D364 optional) |
| To assess cellular and humoral immunogenicity of ChAdOx1 nCoV-19 | a) Interferon-gamma (IFN-y) enzyme-linked immunospot (ELISpot) responses to SARS-CoV-2 spike protein; | See schedule of attendances |
| | b) Enzyme-linked immunosorbent assay (ELISA) to quantify antibodies against SARS-CoV-2 spike protein (seroconversion rates); | |
| | c) virus neutralising antibody (NAb) assays against live and/or pseudotype SARS-CoV-2 virus | |
| Exploratory Objectives Exploratory Immunology | a) Cell analysis by flow cytometry assays b) Functional antibody assays c) Others | See schedule of attendances |

Sample analysis for the completion of exploratory endpoints may be performed under the ethically approved OVC Biobank protocol.

### 5. Trial Design

This is a Phase I/II, single-blinded, placebo-controlled, individually randomised study in healthy adults aged 18-55 years recruited in the UK. ChAdOx1 nCoV-19 or saline placebo will be administered via an intramuscular injection into the deltoid. The study will assess efficacy, safety and immunogenicity of ChAdOx1 nCoV-19.

There will be 3 study groups with 250 volunteers in each vaccine arm (ChAdOx1 nCoV-19 or saline) in groups 1 & 2 combined and 10 participants in group 3 with an overall sample size of 510 (Table 1). Randomisation will take place at an intervention to placebo ratio of 1:1. Only participants enrolled in groups 1 and 2 will be randomised. Participants in group 3 will not be blinded.

Staggered enrolment will apply to the first volunteers receiving the IMP as described in section 7.4.2.2. Participants will be first recruited in groups 1 and 3. Once groups 1 and 3 are fully recruited, subsequent volunteers will be enrolled in group 2.

Safety will be assessed in real time and interim reviews are scheduled after 1, 4, 54 and 100 participants received the IMP. The DSMB will periodically assess safety and efficacy data every 4-8 weeks and/or as required. Participants will be followed over the duration of the study to record adverse events and episodes of virologically confirmed symptomatic COVID-19 cases. A protocol definition of COVID-19 cases will be adopted for the purposes of analysis. COVID-19 cases and related events will be defined as:
a) Fever and/or Upper respiratory tract infection symptoms associated with a positive PCR for SARS-CoV-2
b) Hospital admission associated with a positive PCR for SARS-CoV-2
c) Intensive Care Unit (ICU) admissions associated with a positive PCR for SARS-CoV-2
d) Death associated with a positive PCR for SARS-CoV-2
e) Seroconversion on non-Spike SARS-CoV-2 antigens

Moderate and Severe COVID-19 disease will be defined using clinical criteria. Detailed clinical parameters will be collected from medical records and aligned with agreed definitions as they emerge. These are likely to include, but are not limited to, oxygen saturation, need for oxygen therapy, respiratory rate and other vital signs, need for ventilatory support, Xray and CT scan imaging and blood test results, amongst other clinically relevant parameters.

5.1 Study groups

TABLE 3

Study Groups

| Group | Number of Volunteers | Dose | Route |
|---|---|---|---|
| 1a | 44 | ChAdOx1 nCoV-19 5x10¹⁰ vp | IM |
| 1b | 44 | Saline Placebo | IM |
| 2a | 206 | ChAdOx1 nCoV-19 5x10¹⁰ vp | IM |
| 2b | 206 | Saline Placebo | IM |
| 3 | 10 | Homologous Prime-Boost 5x10¹⁰ vp | IM |

### 5.2 Trial Volunteers

Healthy adult volunteers aged 18-55 will be recruited into the study. Volunteers will be considered enrolled immediately following administration of first vaccination.

### 5.3 Definition of End of Trial

The end of the trial is the date of the last assay conducted on the last sample collected.

### 5.4 Duration of Study

The total duration of the study will be 6 months from the day of enrolment for all volunteers with an optional 12 months follow-up.

### 6.1 Identification of Trial Volunteers

Healthy adults in the UK will be recruited by use of an advertisement +/- registration form formally approved by the ethics committee(s). All volunteers will sign and date the informed consent form before any study specific procedures are performed.

### Inclusion Criteria

The volunteer must satisfy all the following criteria to be eligible for the study:
Healthy adults aged 18-55 years.
Able and willing (in the Investigator’s opinion) to comply with all study requirements.
Willing to allow the investigators to discuss the volunteer’s medical history with their General Practitioner and access all medical records when relevant to study procedures.
For females only, willingness to practice continuous effective contraception (see below) during the study and a negative pregnancy test on the day(s) of screening and vaccination.
Agreement to refrain from blood donation during the course of the study.
Provide written informed consent.

### Exclusion Criteria

The volunteer may not enter the study if any of the following apply:
Prior receipt of any vaccines (licensed or investigational) ≤30 days before enrolment
Planned receipt of any vaccine other than the study intervention within 30 days before and after each study vaccination .
Prior receipt of an investigational or licensed vaccine likely to impact on interpretation of the trial data (e.g. Adenovirus vectored vaccines, any coronavirus vaccines).
Administration of immunoglobulins and/or any blood products within the three months preceding the planned administration of the vaccine candidate.
Any confirmed or suspected immunosuppressive or immunodeficient state, including HIV infection; asplenia; recurrent severe infections and chronic use (more than 14 days) immunosuppressant medication within the past 6 months (inhaled and topical steroids are allowed).
History of allergic disease or reactions likely to be exacerbated by any component of the vaccine.
Any history of hereditary angioedema or idiopathic angioedema.
Any history of anaphylaxis in relation to vaccination.
Pregnancy, lactation or willingness/intention to become pregnant during the study.
History of cancer (except basal cell carcinoma of the skin and cervical carcinoma in situ).
History of serious psychiatric condition likely to affect participation in the study.
Bleeding disorder (e.g. factor deficiency, coagulopathy or platelet disorder), or prior history of significant bleeding or bruising following IM injections or venepuncture.
Any other serious chronic illness requiring hospital specialist supervision.
Suspected or known current alcohol abuse as defined by an alcohol intake of greater than 42 units every week.
Suspected or known injecting drug abuse in the 5 years preceding enrolment.
Any clinically significant abnormal finding on screening biochemistry, haematology blood tests or urinalysis.
Any other significant disease, disorder or finding which may significantly increase the risk to the volunteer because of participation in the study, affect the ability of the volunteer to participate in the study or impair interpretation of the study data.
History of laboratory confirmed COVID-19.

### 6.3.3 Re-Vaccination Exclusion Criteria

The following AEs associated with any vaccine, or identified on or before the day of vaccination constitute absolute contraindications to further administration of an IMP to the volunteer in question. If any of these events occur during the study, the subject will be withdrawn from the study and followed up by the clinical team or their GP until resolution or stabilisation of the event:
Anaphylactic reaction following administration of vaccine
Pregnancy

### 6.1.1 Effective Contraception for Female Volunteers

Female volunteers of childbearing potential are required to use an effective form of contraception during the course of the study (i.e until their last follow-up visit).

### 6.1.2 Prevention of ‘Over Volunteering’

Volunteers will be excluded from the study if they are concurrently involved in another trial where an IMP has been administered within 30 days prior to enrolment, or will be administered during the trial period.

### 6.1.3 Withdrawal of Volunteers

In accordance with the principles of the current revision of the Declaration of Helsinki and any other applicable regulations, a volunteer has the right to withdraw from the study at any time and for any reason, and is not obliged to give his or her reasons for doing so.

If a volunteer withdraws from the study, data and blood samples collected before their withdrawal will still be used on the analysis. Storage of blood samples will continue unless the participant specifically requests otherwise.

In all cases of subject withdrawal, long-term safety data collection, including some procedures such as safety bloods, will continue as appropriate if subjects have received one or more vaccine doses, unless they decline any further follow-up.

### 6.2 Pregnancy

Should a volunteer become pregnant during the trial, no further study IMP will be administered. She will be followed up for clinical safety assessment with her ongoing consent and in addition will be followed until pregnancy outcome is determined.

### 7 Trial Procedures

This section describes the trial procedures for evaluating study participants and follow-up after administration of study vaccine.

### 7.1 Schedule of Attendance

All volunteers in groups 1 will have the same schedule of clinic attendances and procedures as indicated in the schedules of attendance (Table 6). Group 2 will have clinic attendances and procedures as indicated in the schedules of attendances below (tables 7). Group 3 will have clinic attendances and procedures as indicated in the schedules of attendances below (tables 8). Subjects will receive either the ChAdOx1 nCoV-19 vaccine or saline placebo, and undergo follow-up for a total of 6 months with an optional visit at 1 year post enrolment. The total volume of blood donated during the study will be 225 - 420 mL depending on which group they are allocated to. Additional visits or procedures may be performed at the discretion of the investigators, e.g., further medical history and physical examination, urine microscopy in the event of positive urinalysis or additional blood tests if clinically relevant.

### 7.2 Observations

Pulse, blood pressure and temperature will be measured at the time-points indicated in the schedule of procedures and may also be measured as part of a physical examination if indicated at other time-points.

### 7.3 Blood Tests and Urinalysis

Blood will be drawn for the following laboratory tests and processed at agreed NHS Trust laboratories using NHS standard procedures:
Haematology; Full Blood Count
Biochemistry; Sodium, Potassium, Urea, Creatinine, Albumin, Liver Function Tests (ALT, ALP, Bilirubin)
Diagnostic serology; HBsAg, HCV antibodies, HIV antibodies (specific consent will be gained prior to testing blood for these blood-borne viruses)
Immunology; Human Leukocyte Antigen (HLA) typing

Additional safety blood tests may be performed if clinically relevant at the discretion of the medically qualified investigators.

At University of Oxford research laboratories:
Immunology; Immunogenicity will be assessed by a variety of immunological assays. This may include antibodies to SARS-CoV-Spike and non-Spike antigens by ELISA, ex vivo ELISpot assays for interferon gamma and flow cytometry assays, neutralising and other functional antibody assays and B cell analyses. Other exploratory immunological assays including cytokine analysis and other antibody assays, DNA analysis of genetic polymorphisms potentially relevant to vaccine immunogenicity and gene expression studies amongst others may be performed at the discretion of the Investigators.
Urinalysis; Urine will be tested for protein, blood and glucose at screening. For female volunteers only, urine will be tested for beta-human chorionic gonadotrophin (β-HCG) at screening and immediately prior to vaccination.

Samples that are to be stored for future research will be transferred to the OVC Biobank (REC 16/SC/0141).

### 7.4 Study Visits

The procedures to be included in each visit are documented in the schedule of attendances (Tables6-8).

### 7.4.1 Screening Visit

All potential volunteers will have a screening visit, which may take place up to 90 days prior to vaccination. Informed consent will be taken before screening.

If eligible, a day 0 visit will be scheduled for the volunteer to receive the vaccine and subsequent follow-up.

### 7.4.2 Day 0: Enrolment and Vaccination Visit

Volunteers will be considered enrolled in to the trial at the point of vaccination. Before vaccination/trial intervention, the eligibility of the volunteer will be reviewed. Pulse, blood pressure and temperature will be observed and if necessary, a medical history and physical examination may be undertaken to determine need to postpone vaccination. Vaccinations will be administered as described below.

### 7.4.2.1 Vaccination

All vaccines and saline placebo injections will be administered intramuscularly according to specific SOPs. The injection site will be covered with a sterile dressing and the volunteer will stay in the trial site for observation, in case of immediate adverse events. Observations will be taken 60 minutes after vaccination (+/- 30 minutes) and the sterile dressing removed and injection site inspected.

In all groups, volunteers will be given an oral thermometer, tape measure and diary card (paper or electronic), with instructions on use, along with the emergency 24 hour telephone number to contact the on-call study physician if needed. Volunteers will be instructed on how to self-assess the severity of these AEs. There will also be space on the diary card to self-document unsolicited AEs, and whether medication was taken to relieve the symptoms. Diary cards will collect information on the timing and severity of the following solicited AEs:

TABLE 4

Solicited AEs as collected on post vaccination diary cards

| | |
|---|---|
| Local solicited AEs | Systemic solicited AEs |
| Pain | Fever |
| Tenderness | Feverishness |
| | Chills |
| Redness | Joint pains |
| Warmth | Muscle pains |
| Itch | Fatigue |
| Swelling | Headache |
| Induration | Malaise |
| | Nausea |

### 7.4.2.2 Sequence of Enrolment and Vaccination of Volunteers

Prior to initiation of the study, any newly available safety data will be reviewed from animal studies or clinical trials of coronavirus vaccines being tested elsewhere, and discussed with the DSMB and/or MHRA as necessary. For safety reasons, the first volunteer to receive the IMP will be vaccinated ahead of any other participants and the profile of adverse events will be reviewed after 48 hours (±24 h) post vaccination. Provided there are no safety concerns, as assessed by the investigators and/or chair of DSMB, another 3 volunteers will be vaccinated with the IMP after at least 48 hours (±24 h) has elapsed following first vaccination and at least 1 hour apart from each other. The profile of AEs will be assessed by medically qualified investigators in real time and after 48 hours (±24 h) of the first 4 participants receiving the IMP, further vaccinations will proceed provided there are no safety concerns. Relevant investigators and chair of DSMB will be asked to provide a decision on whether further vaccinations can go ahead after the first 4 participants received the IMP. A full DSMB may also be consulted should safety concerns arise at this point.

A review will be conducted based on accumulated safety data of the first 54 participants receiving the IMP. Enrolment of up to 100 participants will only proceed if the CI, and/or other designated relevant investigators and the chair of DSMB assess the data as indicating that it is safe to do so. At this point, any new immunopathology data from pre-clinical challenge studies in ferrets and non-human primates will be assessed by the CI and/or other designated relevant investigators and the DSMB prior to enrolment of up to 100 participants.

A second review will be conducted based on accumulated safety data on 100 participants receiving the IMP before enrolling the remainder of participants in the study. Enrolment of the remaining 160 participants receiving the IMP will only proceed if the CI, and/or other designated relevant investigators and the DSMB assess the data as indicating that it is safe to do so. The table below provides an estimate of the sequence of recruitment

Should other batches of IMP become available, the same staggered enrolment procedures will apply to these new batches.

### 7.4.3 Subsequent Visits

Follow-up visits will take place as per the schedule of attendances described in tables 6-8 with their respective windows. Volunteers will be assessed for local and systemic adverse events, interim history, physical examination, review of diary cards (paper or electronic) and blood tests at these time points as detailed in the schedule of attendances. Blood will also be taken for immunology purposes.

If volunteers experience adverse events (laboratory or clinical), which the investigator (physician), CI and/or DSMB chair determine necessary for further close observation, the volunteer may be admitted to an NHS hospital for observation and further medical management under the care of the Consultant on call.

### 7.4.4 Participants Under Quarantine

Given the evolving epidemiological situation both globally and in the UK, should a participant be under quarantine and unable to attend any of the scheduled visits, a telephone consultation will be arranged in order to obtain core study data where possible.

### 7.4.5 Changes to Group Numbers

Should other batches of IMP be required in order to complete dosing in the trial, the same staggered enrolment procedures described in section 7.4.2.2 will apply. In this case, the sample size in group 1 will be increased by 88 participants per new batch, without increasing the overall sample size (i.e number of participants in group 2 will be reduced by the same number). This is to ensure safety and immunogenicity data are comparable across different batches.

TABLE 6

Schedule of attendances for participants in group 1

| | | | | | | | | | |
|---|---|---|---|---|---|---|---|---|---|
| Attendance Number | 1^{S} | 2 | 3 | 4 | 5 | 6 | 7 | 8 | 9 |
| Timeline^{∗∗} (days) | ≤90 | 0 | 2 | 7 | 14 | 28 | 56 | 182 | 364 (Optiona 1) |
| Time window (days) | | | ±1 | ±2 | ±3 | ±7 | ±7 | ±14 | ±30 |
| Informed Consent | X | | | | | | | | |
| Review contraindications, inclusion and exclusion criteria | X | X | | | | | | | |
| Vaccination | | X | | | | | | | |
| Vital signs^ | X | X | X | X | X | X | X | X | X |
| Ascertainment of adverse events | | X | X | X | X | X | X | X | X |
| Diary cards provided | | X | | | | | | | |
| Diary cards collected | | | | | | X | | | |
| Medical History, Physical Examination | X | (X ) | (X ) | (X ) | (X) | (X) | (X) | (X) | (X) |
| Biochemistry, Haematology (mL) | 5 | 5 | 5 | 5 | | 5 | | | |
| Exploratory immunology (mL) | | 50 | | 10 | 50 | 50 | 50 | 50 | 50 |
| Urinalysis | X | | | | | | | | |
| Urinary bHCG (women only) | X | X | | | | | | | |
| HLA typing (mL) | | 4 | | | | | | | |
| HBsAg, HCV Ab, HIV serology (mL) | 5 | | | | | | | | |
| Blood volume per visit | 10 | 59 | 5 | 15 | 50 | 55 | 50 | 50 | 50 |
| Cumulative blood volume^{%} | 10 | 69 | 74 | 89 | 139 | 194 | 244 | 294 | 344 |

S = screening visit; (X) = if considered necessary ^ = Vital signs includes pulse, blood pressure and temperature;
** Timeline is approximate only. Exact timings of visits relate to the day on enrolment, ie, each visit must occur at indicated number of days after enrolment ± time window.
% Cumulative blood volume for Oxford volunteers if blood taken as per schedule, and excluding any repeat safety blood test that may be necessary.

TABLE 7

Schedule of attendances for participants in group 2

| | | | | | |
|---|---|---|---|---|---|
| Attendance Number | 1^{S} | 2 | 3 | 4 | 5 |
| Timeline^{∗∗} (days) | ≤ 90 | 0 | 28 | 182 | 364 (Optional) |
| Time window (days) | | | ±7 | ±14 | ±30 |
| Informed Consent | X | | | | |
| Review contraindications, inclusion and exclusion criteria | X | X | | | |
| Vaccination | | X | | | |
| Vital signs^ | X | X | X | X | X |
| Ascertainment of adverse events | | X | X | X | X |
| Diary cards provided | | X | | | |
| Diary cards collected | | | X | | |
| Medical History, Physical Examination | X | (X) | (X) | (X) | (X) |
| Biochemistry, Haematology (mL) | 5 | 5 | 5 | | |
| Exploratory immunology (mL) | | 50 | 50 | 50 | 50 |
| Urinalysis | X | | | | |
| Urinary bHCG (women only) | X | X | | | |
| HLA typing (mL) | | 4 | | | |
| HBsAg, HCV Ab, HIV serology (mL) | 5 | | | | |
| Blood volume per visit | 10 | 59 | 55 | 50 | 50 |
| Cumulative blood volume^{%} | 10 | 69 | 124 | 174 | 224 |

S = screening visit; (X) = if considered necessary ^ = Vital signs includes pulse, blood pressure and temperature;
** Timeline is approximate only. Exact timings of visits relate to the day on enrolment, ie, each visit must occur at indicated number of days after enrolment ± time window.
% Cumulative blood volume for Oxford volunteers if blood taken as per schedule, and excluding any repeat safety blood test that may be necessary.

TABLE 8

Schedule of attendances for participants in group 3

| | | | | | | | | | | | | |
|---|---|---|---|---|---|---|---|---|---|---|---|---|
| Attendance Number | 1^{S} | 2 (V1) | 3 | 4 | 5 | 6 (V2) | 7 | 8 | 9 | 10 | 11 | 12 |
| Timeline^{∗∗} (days) | ≤90 | 0 | 2 | 7 | 14 | 28 | 30 | 35 | 42 | 56 | 18 2 | 364 (optional) |
| Time window (days) | | | ±1 | ±3 | ±3 | ±7 | ±1 | ±2 | ±3 | ±3 | ±1 4 | ±30 |
| Informed Consent | X | | | | | | | | | | | |
| Review contraindications, inclusion and exclusion criteria | X | X | | | | X | | | | | | |
| Vaccination | | X | | | | X | | | | | | |
| Vital signs^ | X | X | X | X | X | X | X | X | X | X | X | X |
| Ascertainment of adverse events | | X | X | X | X | X | X | X | X | X | X | X |
| Diary cards provided | | X | | | | X | | | | | | |
| Diary cards collected | | | | | | X | | | | X | | |
| Medical History, Physical Examination | X | (X) | (X) | (X) | (X) | (X) | (X) | (X) | (X) | (X) | (X) | (X) |
| Biochemistry⁵⁵, Haematology (mL) | 5 | 5 | 5 | 5 | | 5 | 5 | 5 | | 5 | | |
| Exploratory immunology^{£} (mL) | | 50 | | 10 | 50 | 50 | | 10 | 50 | 50 | 50 | 50 |
| Urinalysis | X | | | | | | | | | | | |
| Urinary bHCG (women only) | X | X | | | | X | | | | | | |
| HLA typing (mL) | | 4 | | | | | | | | | | |
| HBsAg, HCV Ab, HIV serology (mL) | 5 | | | | | | | | | | | |
| Blood volume per visit | 10 | 59 | 5 | 15 | 50 | 55 | 5 | 15 | 50 | 55 | 50 | 50 |
| Cumulative blood volume^{%} | 10 | 69 | 74 | 89 | 13 9 | 194 | 19 9 | 21 4 | 26 4 | 31 9 | 36 9 | 419 |

S = screening visit; (X) = if considered necessary ^ = Vital signs includes pulse, blood pressure and temperature;
** Timeline is approximate only. Exact timings of visits relate to the day on enrolment, ie, each visit must occur at indicated number of days after enrolment ± time window.
% Cumulative blood volume for Oxford volunteers if blood taken as per schedule, and excluding any repeat safety blood test that may be necessary.

### 7.4.3 Symptomatic Volunteers

Participants who become symptomatic during follow-up will be instructed to call the study team who will then advise on how to proceed with clinical testing for COVID-19, as per the trial working instructions. Participants will get weekly reminders (email or text messages) to get in touch with the study team if they present with a fever or upper respiratory tract symptoms and if they are admitted to hospital for any reason.

### 7.4.4 Medical Notes Review

With the participants consent, the study team will request access to medical notes or submit a data collection form for completion by attending clinical staff on any medically attended COVID-19 episodes. Any data which are relevant to ascertainment of efficacy endpoints and disease enhancement (AESI) will be collected. These are likely to include, but not limited to, information on ICU admissions, clinical parameters such as oxygen saturation, respiratory rates and vital signs, need for oxygen therapy, need for ventilatory support, imaging and blood tests results, amongst others.

### 7.4.5 Randomisation, Blinding and Code-Breaking

Participants will be randomised to investigational vaccine or saline placebo in a 1:1 allocation, using block randomisation. Block sizes will reflect the numbers to be recruited at each stage of the study. The first block will be a block of 2 participants, followed by a block of 6, then further combination of blocks of 2, 6, or 10 as required to meet the totals for randomisation for each day.

Participants enrolled in groups 1 and 2 will be blinded to the arm they have been allocated to, whether investigational vaccine or placebo. The trial staff administering the vaccine will not be blinded. Vaccines will be prepared out of sight of the participant and syringes will be covered with an opaque object/material until ready for administration to ensure blinding.

If the clinical condition of a participant necessitates breaking the code, this will be undertaken according to a trial specific working instruction and group allocation sent to the attending physician, if unblinding is thought to be relevant and likely to change clinical management.

Participants enrolled in group 3 will not be blinded

### 7.1 Manufacturing and Presentation

### 7.1.1 Description of ChAdOx1 nCoV-19

ChAdOx1 nCoV-19 vaccine consists of the replication-deficient simian adenovirus vector ChAdOx1, containing the structural surface glycoprotein (Spike protein) antigens of SARS-CoV-2.

### 7.2 Supply

ChAdOx1 nCoV-19 has been formulated and vialed at the Clinical Biomanufacturing Facility (CBF), University of Oxford. At the CBF the vaccine will be certified and labelled for the trial by a Qualified Person (QP) before transfer to the clinical site.

### 7.3 Storage

The vaccine is stored at nominal -80° C. in a locked freezer, at the clinical site. All movements of the study vaccines will be documented in accordance with existing standard operating procedure (SOP). Vaccine accountability, storage, shipment and handling will be in accordance with relevant SOPs and forms.

### 7.4 Administration

On vaccination day, ChAdOx1 nCoV-19 will be allowed to thaw to room temperature and will be administered within 1 hour of removal from the freezer. The vaccine will be administered intramuscularly into the deltoid of the non-dominant arm (preferably). All volunteers will be observed in the unit for 1 hour (±30 minutes) after vaccination. During administration of the investigational products, Advanced Life Support drugs and resuscitation equipment will be immediately available for the management of anaphylaxis. Vaccination will be performed and the IMPs handled according to the relevant SOPs.

### 8.5 Rationale for Selected Dose

The dose to be administered in this trial have been selected on the basis of clinical experience with the ChAdOx1 adenovirus vector expressing different inserts and other similar adenovirus vectored vaccines (eg. ChAd63).

A first-in-man dose escalation study using the ChAdOx1 vector encoding an influenza antigen (FLU004), safely administered ChAdOx1 NP+M1 at doses ranging from 5 x 10⁸ to 5 x 10¹⁰ vp. Subsequent review of the data identified an optimal dose of 2.5 x 10¹⁰ vp balancing immunogenicity and reactogenicity. This dose has subsequently been given to over hundreds of volunteers in numerous larger phase 1 studies at the Jenner Institute. ChAdOx1 vectored vaccines have thus far demonstrated to be very well tolerated. The vast majority of AEs have been mild-moderate and there have been no SARs until this date.

Another simian adenovirus vector (ChAd63) has been safely administered at doses up to 2 x 10¹¹ vp with an optimal dose of 5 x 10¹⁰ vp, balancing immunogenicity and reactogenicity.

MERS001 was the first clinical trial of a ChAdOx1 vectored expressing the full-length Spike protein from a separate, but related betacoronavirus. ChAdOx1 MERS has been given to 31 participants to date at doses ranging from 5x10⁹ vp to 5x10¹⁰ vp. Despite higher reactogeniticy observed at the 5x10¹⁰ vp, this dose was safe, with self-limiting AEs and no SARs recorded. The 5x10¹⁰ vp was the most immunogenic, in terms of inducing neutralising antibodies against MERS-CoV using a live virus assay (Folegatti et al. Lancet Infect Dis, 2020, in press). Given the immunology findings and safety profile observed with a ChAdOx1 vectored vaccine against MERS-CoV, the 5x10¹⁰ vp dose was chosen for ChAdOx1 nCoV-19.

As this is a first-in-human assessment of the SARS-CoV-2 S antigenic insert, a staggered enrolment will apply for the first volunteers enrolled in the study. The same procedure will apply, should other batches of ChAdOx1 nCoV-19 become available. Safety of ChAdOx1 nCoV-19 will be monitored in real time and should unacceptable adverse events or safety concerns arise, doses will be decreased.

### 8.7 Placebo

Participants who are allocated to the control group will receive a placebo injection of 0.9% saline instead of ChAdOx1 nCoV-19. The volume and site of injection will be the same as for the intervention arm and participants will be blinded as to which injection they are receiving. Each saline pod will only be used for a single participant. A vaccine accountability log of the saline will be maintained at each trial site.

### 8.10 Concomitant Medication

As set out by the exclusion criteria, volunteers may not enter the study if they have received: any vaccine in the 30 days prior to enrolment or there is planned receipt of any other vaccine within 30 days of each vaccination, any investigational product within 30 days prior to enrolment or if receipt is planned during the study period, or if there is any chronic use (>14 days) of any immunosuppressant medication within 6 months prior to enrolment or if receipt is planned at any time during the study period (inhaled and topical steroids are permitted).

### 9 Assessment of Safety

Safety will be assessed by the frequency, incidence and nature of AEs and SAEs arising during the study.

### 9.1 Definitions

### 9.1.1 Adverse Event (AE)

An AE is any untoward medical occurrence in a volunteer, which may occur during or after administration of an IMP and does not necessarily have a causal relationship with the intervention. An AE can therefore be any unfavourable and unintended sign (including any clinically significant abnormal laboratory finding or change from baseline), symptom or disease temporally associated with the study intervention, whether or not considered related to the study intervention.

### 9.1.2 Adverse Reaction (AR)

An AR is any untoward or unintended response to an IMP. This means that a causal relationship between the IMP and an AE is at least a reasonable possibility, i.e., the relationship cannot be ruled out. All cases judged by the reporting medical Investigator as having a reasonable suspected causal relationship to an IMP (i.e. possibly, probably or definitely related to an IMP) will qualify as AR.

Adverse events that may be related to the IMP are listed in the Investigator’s Brochure for each product.

### 9.1.4 Serious Adverse Event (SAE)

An SAE is an AE that results in any of the following outcomes, whether or not considered related to the study intervention.
Death
Life-threatening event (i.e., the volunteer was, in the view of the Investigator, at immediate risk of death from the event that occurred). This does not include an AE that, if it occurred in a more severe form, might have caused death.
Persistent or significant disability or incapacity (i.e., substantial disruption of one’s ability to carry out normal life functions).
Hospitalisation or prolongation of existing hospitalisation, regardless of length of stay, even if it is a precautionary measure for continued observation. Hospitalisation (including inpatient or outpatient hospitalisation for an elective procedure) for a pre-existing condition that has not worsened unexpectedly does not constitute a serious AE.
An important medical event (that may not cause death, be life threatening, or require hospitalisation) that may, based upon appropriate medical judgment, jeopardise the volunteer and/or require medical or surgical intervention to prevent one of the outcomes listed above. Examples of such medical events include allergic reaction requiring intensive treatment in an emergency room or clinic, blood dyscrasias, or convulsions that do not result in inpatient hospitalisation.
Congenital anomaly or birth defect.

### 9.1.5 Serious Adverse Reaction (SAR)

An AE that is both serious and, in the opinion of the reporting Investigator or Sponsors, believed to be possibly, probably or definitely due to an IMP or any other study treatments, based on the information provided.

### 9.1.6 Suspected Unexpected Serious Adverse Reaction (SUSAR)

A SAR, the nature and severity of which is not consistent with the information about the medicinal product in question set out in the IB.

### 9.2 Expectedness

No IMP related SAEs are expected in this study. All SARs will therefore be reported as SUSARs.

### 9.3 Foreseeable Adverse Reactions

The foreseeable ARs following vaccination with ChAdOx1 nCoV-19 include injection site pain, tenderness, erythema, warmth, swelling, induration, pruritus, myalgia, arthralgia, headache, fatigue, fever, feverishness, chills, malaise and nausea.

### 9.4 Adverse Events of Special Interest

Disease enhancement following vaccination with ChAdOx1 nCoV-19 will be monitored. Severe COVID-19 disease will be defined using clinical criteria. Detailed clinical parameters will be collected from medical records and aligned with agreed definitions as they emerge. These are likely to include, but are not limited to, oxygen saturation, need for oxygen therapy, respiratory rate, need for ventilatory support, imaging and blood test results, amongst other clinically relevant parameters.

### 9.5 Causality

For every AE, an assessment of the relationship of the event to the administration of the vaccine will be undertaken by the CI-delegated clinician. An interpretation of the causal relationship of the intervention to the AE in question will be made, based on the type of event; the relationship of the event to the time of vaccine administration; and the known biology of the vaccine therapy (Table 9). Alternative causes of the AE, such as the natural history of pre-existing medical conditions, concomitant therapy, other risk factors and the temporal relationship of the event to vaccination will be considered and investigated. Causality assessment will take place during planned safety reviews, interim analyses (e.g. if a holding or stopping rule is activated) and at the final safety analysis, except for SAEs, which should be assigned by the reporting investigator, immediately, as described in SOP OVC005 Safety Reporting for CTIMPs.

TABLE 9

Guidelines for assessing the relationship of vaccine administration to an AE.

| | | |
|---|---|---|
| 0 | No Relationship | No temporal relationship to study product and Alternate aetiology (clinical state, environmental or other interventions); and Does not follow known pattern of response to study product |
| 1 | Unlikely | Unlikely temporal relationship to study product and Alternate aetiology likely (clinical state, environmental or other interventions) and Does not follow known typical or plausible pattern of response to study product. |
| 2 | Possible | Reasonable temporal relationship to study product; or Event not readily produced by clinical state, environmental or other interventions; or Similar pattern of response to that seen with other vaccines |
| 3 | Probable | Reasonable temporal relationship to study product; and Event not readily produced by clinical state, environment, or other interventions or Known pattern of response seen with other vaccines |
| 4 | Definite | Reasonable temporal relationship to study product; and Event not readily produced by clinical state, environment, or other interventions; and Known pattern of response seen with other vaccines |

### 9.6 Reporting Procedures for All Adverse Events

All local and systemic AEs occurring in the 28 days following each vaccination observed by the Investigator or reported by the volunteer, whether or not attributed to study medication, will be recorded in electronic diaries or study database. All AEs that result in a volunteer’s withdrawal from the study will be followed up until a satisfactory resolution occurs, or until a non-study related causality is assigned (if the volunteer consents to this). SAEs and Adverse Events of Special Interest will be collected throughout the entire trial period.

### 9.7 Assessment of Severity

The severity of clinical and laboratory adverse events will be assessed according to scales in Table 12, based on FDA toxicity grading scales for healthy and adolescent volunteers enrolled in preventive vaccine clinical trials.

TABLE 10

Severity grading criteria for local adverse events *erythema ≤2.5 cm is an expected consequence of skin puncture and will therefore not be considered an adverse event

| Adverse Event | Grade | Intensity |
|---|---|---|
| Pain at injection site | 1 | Pain that is easily tolerated |
| | 2 | Pain that interferes with daily activity |
| | 3 | Pain that prevents daily activity |
| | 4 | A&E visit or hospitalisation |
| Tenderness | 1 | Mild discomfort to touch |
| | 2 | Discomfort with movement |
| | 3 | Significant discomfort at rest |
| | 4 | A&E visit or hospitalisation |
| Erythema at injection site* | 1 | 2.5 - 5 cm |
| | 2 | 5.1 - 10 cm |
| | 3 | >10 cm |
| | 4 | Necrosis or exfoliative dermatitis |
| Induration/Swelling at injection site | 1 | 2.5 - 5 cm and does not interfere with activity |
| | 2 | 5.1 - 10 cm or interferes with activity |
| | 3 | >10 cm or prevents daily activity |
| | 4 | Necrosis |

TABLE 11

Severity grading criteria for physical observations. *Taken after ≥10 minutes at rest **When resting heart rate is between 60 — 100 beats per minute. Use clinical judgement when characterising bradycardia among some healthy subject populations, for example, conditioned athletes. ***Only if symptomatic (e.g. dizzy/light-headed)

| Vital Signs | Grade 1 (mild) | Grade 2 (moderate) | Grade 3 (severe) | Grade 4 Potentially Life threatening |
|---|---|---|---|---|
| Fever (oral) | 38.0° C. - 38.4° C. | 38.5° C. ― 38.9° C. | 39.0° C. - 40° C. | > 40° C. |
| Tachycardia (bpm)* | 100-115 | 116-130 | >130 | A&E visit or hospitalisation for arrhythmia |
| Bradycardia (bpm)** | 50-54 | 45-49 | <45 | A&E visit or hospitalisation for arrhythmia |
| Systolic hypertension (mmHg) | 141-150 | 151-155 | ≥155 | A&E visit or hospitalization for malignant hypertension |
| Diastolic hypertension (mmHg) | 91-95 | 96-100 | >100 | A&E visit or hospitalization for malignant hypertension |
| Systolic hypotension (mmHg)*** | 85-89 | 80-84 | <80 | A&E visit or hospitalization for hypotensive shock |
| Respiratory Rate – breaths per minute | 17-20 | 21-25 | >25 | Intubation |

TABLE 12

Severity grading criteria for local and systemic AEs.

| | |
|---|---|
| GRADE 0 | None |
| GRADE 1 | Mild: Transient or mild discomfort (< 48 hours); No interference with activity; No medical intervention/therapy required |
| GRADE 2 | Moderate: Mild to moderate limitation in activity ― some assistance may be needed; no or minimal medical intervention/therapy required |
| GRADE 3 | Severe: Marked limitation in activity, some assistance usually required; medical intervention/therapy required. |
| GRADE 4 | Potentially Life-threatening: requires assessment in A&E or hospitalisation |

Solicited local adverse events:
   If more than 25% of doses of the vaccine at a given time point (e.g. Day 0, Day 28) in a study group are followed by the same Grade 3 solicited local adverse event beginning within 2 days after vaccination (day of vaccination and one subsequent day) and persisting at Grade 3 for >72 hrs
Solicited systemic adverse events:
   If more than 25% of doses of the vaccine at a given time point (e.g. Day 0, Day 28) in a study group are followed by the same Grade 3 solicited systemic adverse event beginning within 2 days after vaccination (day of vaccination and one subsequent day) and persisting at Grade 3 for >72 hrs
Unsolicited adverse events:
   If more than 25% of doses of the vaccine at a given time point (e.g. Day 0, Day 28) in a study group are followed by the same Grade 3 unsolicited adverse event beginning within 2 days after vaccination (day of vaccination and one subsequent day) and persisting at Grade 3 for >72 hrs
Laboratory adverse event:
   If more than 25% of doses of the vaccine at a given time point (e.g. Day 0, Day 28) in a study group are followed by the same Grade 3 laboratory adverse event beginning within 2 days after vaccination (day of vaccination and one subsequent day) and persisting at Grade 3 for >72 hrs

### 9.14.2 Individual Stopping Rules (Will Apply to Prime-Boost Group Only)

In addition to the above stated group holding rules, stopping rules for individual volunteers will apply (i.e., indications to withdraw individuals from further vaccinations). Study participants who present with at least one of the following stopping rules will be withdrawn from further vaccination in the study:
Local reactions: Injection site ulceration, abscess or necrosis
Laboratory AEs: the volunteer develops a Grade 3 laboratory AE considered possibly, probably or definitely related within 7 days after vaccination and persisting continuously at Grade 3 for > 72 hrs.
Systemic solicited adverse events:
   the volunteer develops a Grade 3 systemic solicited AE considered possibly, probably or definitely related within 2 days after vaccination (day of vaccination and one subsequent day) and persisting continuously at Grade 3 for > 72 hrs.
Unsolicited adverse events:
   the volunteer has a Grade 3 adverse event, considered possibly, probably or definitely related to vaccination, persisting continuously at Grade 3 for >72 hrs.
   the volunteer has a SAE considered possibly, probably or definitely related to vaccination.
   the volunteer has an acute allergic reaction or anaphylactic shock following the administration of vaccine investigational product.

If a volunteer has an acute respiratory illness (moderate or severe illness with or without fever) or a fever (oral temperature greater than 37.8° C.) at the scheduled time of administration of investigational product/placebo, the volunteer will not be enrolled and will be withdrawn from the study.

### 10 Statistics

### 10.1 Description of Statistical Methods

A fully detailed statistical analysis plan will be developed and signed by the chief investigator prior to any data analysis being conducted. In brief, the analysis will incorporate the following;

### 10.1.1 Primary Efficacy

The primary efficacy analysis endpoints include: PCR positive COVID-19 symptomatic cases captured by the National Health Service.

Only events that occur more than 14 days after vaccination will be included in efficacy evaluations to allow time for the vaccine recipients to mount a protective immune response and to provide a more accurate estimation of VE. Vaccine efficacy (VE) will be calculated as (1 - RR) x 100%, where RR is the relative risk of symptomatic infection (ChADOx1 nCOV-19: Control) and 95% confidence intervals will be presented.

Cumulative incidence of symptomatic infections will be presented using the Kaplan-Meier method

### 10.1.2 Primary Safety

All SAEs will be presented for each group using descriptive analyses.

### 10.1.3 Secondary Efficacy

The secondary efficacy analysis endpoints include;
1. Hospital admissions with PCR positive COVID-19
2. Intensive Care Unit admissions with PCR positive COVID-19
3. Death due to PCR positive COVID-19 infection
4. Seroconversion to non-Spike SARS-CoV-2 antigens

Secondary efficacy endpoints will be analysed in the same way as the primary endpoints.

### 10.1.4 Safety & Reactogenicity

Counts and percentages of each local and systemic solicited adverse reaction from diary cards, and all unsolicited AEs, and SAEs of special interest will be presented for each group.

### 10.1.5 Immunogenicity

Highly skewed ELISA data will be log-transformed prior to analysis. The geometric mean concentration and associated 95% confidence interval will be summarised for each group at each timepoint, by computing the anti-log of the mean difference of the log-transformed data.

Comparisons between groups will be made using a Mann Whitney U test.

### 10.2 The Number of Participants

The study is powered to detect a difference in proportions with symptomatic infection with COVID-19 between those receiving investigational vaccine and control.

If the attack rate for symptomatic COVID-19 infections during the trial is 10% in the control group during the efficacy evaluation period (after the first 14 days of the study), then the study will have 90% power (5% alpha) to detect a minimum vaccine efficacy of 74%. A higher attack rate of 20% will enable detection of vaccine efficacy as low as 53%. These calculations assume no loss to follow up as it is likely that the participants will be motivated to remain engaged in the study, and the time period for follow up is relatively short.

Group 3 participants receiving prime-boost vaccination will not be included in the efficacy evaluation as there will be too few participants in this group to enable accurate estimation of the event rate after a prime-boost vaccine schedule.

### 10.3 Combined Analyses

The Phase I/II study (COV001) and the phase ⅔ study (COV002) are expected to be running concurrently during a period of high disease incidence in the UK. Efficacy data from both studies will be combined in a prospective meta-analysis to enable more precise estimation of efficacy and safety parameters.

| Total sample size* | N1 Control | N2 nCOV -19 | Ratio Vac: Control | Attack rate control | Attack rate nCOV -19 | Minimu m VE that could be detected | Expected number cases in control group | Expected number cases in nCOV-19 group |
|---|---|---|---|---|---|---|---|---|
| 500 | 250 | 250 | 1 | 0.1 | 0.026 | 0.74 | 25 | 6 |
| 500 | 250 | 250 | 1 | 0.2 | 0.094 | 0.53 | 50 | 23 |

### 10.3 Procedure for Accounting for Missing, Unused, and Spurious Data

All available data will be included in the analysis

### 10.4 Inclusion in Analysis

All vaccinated participants will be included in the analysis and will be analysed according to vaccine received.

### 11.5 Data Quality

Data collection tools will undergo appropriate validation to ensure that data are collected accurately and completely. Datasets provided for analysis will be subject to quality control processes to ensure analysed data is a true reflection of the source data.

Trial data will be managed in compliance with local data management SOPs. If additional, study specific processes are required, an approved Data Management Plan will be implemented

### Quality Control and Quality Assurance Procedures

### 11.1 Investigator Procedures

Approved site-specific standard operating procedures (SOPs) will be used at all clinical and laboratory sites.

### 11.2 Monitoring

Regular monitoring will be performed according to GCP by the monitor.

### 14.1 Declaration of Helsinki

The Investigators will ensure that this study is conducted according to the principles of the current revision of the Declaration of Helsinki.

### 15. References to Example 11

1. Zhu, N., et al., A Novel Coronavirus from Patients with Pneumonia in China, 2019. New England Journal of Medicine, 2020. 382(8): p. 727-733.

2. Lu, R., et al., Genomic characterisation and epidemiology of 2019 novel coronavirus: implications for virus origins and receptor binding. Lancet (London, England), 2020. 395(10224): p. 565-574.

3. Li, F., Structure, Function, and Evolution of Coronavirus Spike Proteins. Annual review of virology, 2016. 3(1): p. 237-261.

4. Zhou, P., et al., A pneumonia outbreak associated with a new coronavirus of probable bat origin. Nature, 2020.

5. Alharbi, N.K., et al., ChAdOx1 and MVA based vaccine candidates against MERS-CoV elicit neutralising antibodies and cellular immune responses in mice. Vaccine, 2017. 35(30): p. 3780-3788.

6. Antrobus, R.D., et al., Clinical assessment of a novel recombinant simian adenovirus ChAdOx1 as a vectored vaccine expressing conserved Influenza A antigens. Mol Ther, 2014. 22(3): p. 668-74.

7. Coughlan, L., et al., Heterologous Two-Dose Vaccination with Simian Adenovirus and Poxvirus Vectors Elicits Long-Lasting Cellular Immunity to Influenza Virus A in Healthy Adults. EBioMedicine, 2018.

8. Wilkie, M., et al., A phase I trial evaluating the safety and immunogenicity of a candidate tuberculosis vaccination regimen, ChAdOx1 85A prime - MVA85A boost in healthy UK adults. Vaccine, 2020. 38(4): p. 779-789.

9. Modjarrad, K., MERS-CoV vaccine candidates in development: The current landscape. Vaccine, 2016. 34(26): p. 2982-7.

10. Tseng, C.T., et al., Immunization with SARS coronavirus vaccines leads to pulmonary immunopathology on challenge with the SARS virus. PLoS One, 2012. 7(4): p. e35421.

11. Weingartl, H., et al., Immunization with modified vaccinia virus Ankara-based recombinant vaccine against severe acute respiratory syndrome is associated with enhanced hepatitis in ferrets. J Virol, 2004. 78(22): p. 12672-6.

12. Liu, L., et al., Anti-spike IgG causes severe acute lung injury by skewing macrophage responses during acute SARS-CoV infection. JCI insight, 2019. 4(4): p. e123158.

13. Agrawal, A.S., et al., Immunization with inactivated Middle East Respiratory Syndrome coronavirus vaccine leads to lung immunopathology on challenge with live virus. Hum Vaccin Immunother, 2016. 12(9): p. 2351-6.

14. Munster, V.J., et al., Protective efficacy of a novel simian adenovirus vaccine against lethal MERS-CoV challenge in a transgenic human DPP4 mouse model. NPJ Vaccines, 2017. 2: p. 28.

15. Alharbi, N.K., et al., Humoral Immunogenicity and Efficacy of a Single Dose of ChAdOx1 MERS Vaccine Candidate in Dromedary Camels. Sci Rep, 2019. 9(1): p. 16292.

ChAdOx1 SARS-CoV2 is thus demonstrated to be a plausible and credible vaccine for humans.

### Example 12: Demonstration in Mammals

In this example the advantageous effects of the invention are demonstrated in mammals. In this example the mammals are mice.

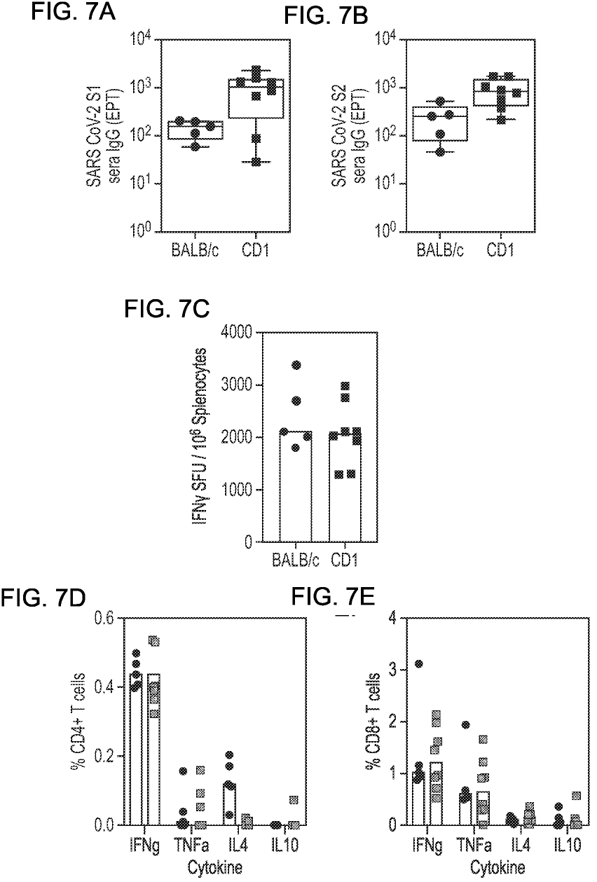

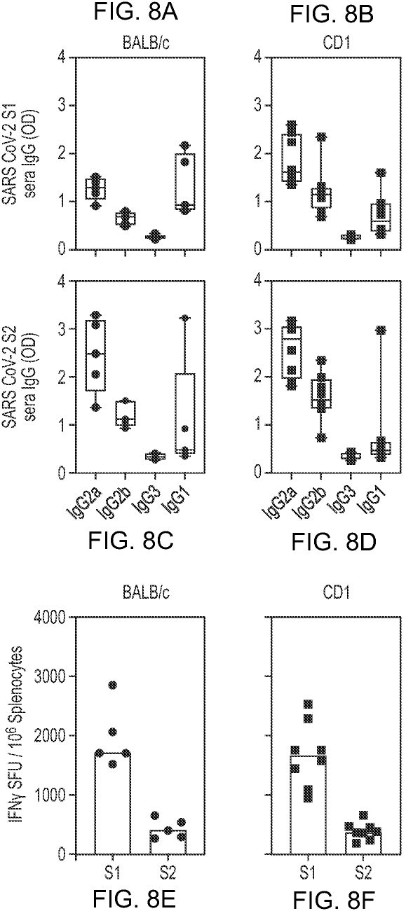

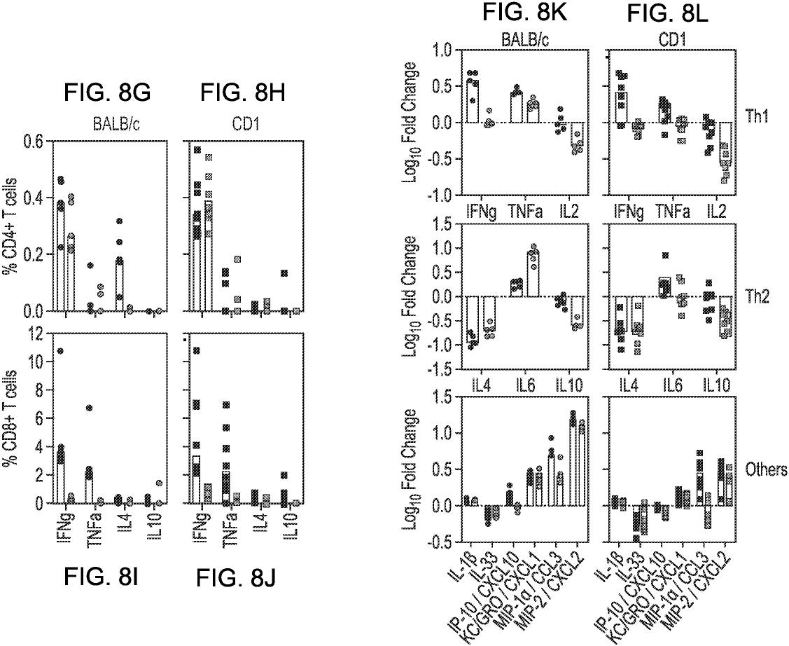

Two mouse strains (BALB/c, N=5 and outbred CD1, N=8) were vaccinated intramuscularly (IM) with ChAdOx1 nCoV-19 (for construction/assembly of ChAdOx1 nCoV-19 see above, especially the examples). Detailed serology and in-depth cellular immunity was profiled 14 days later. Data are presented in FIG. 7 and FIG. 8.

FIG. 7 shows antigen specific responses following ChAdOx1 nCov19 vaccination in mice (i.e. administration of the composition of the invention to mice). BALB/c and outbred (CD1) mice were intramuscularly administered with 10⁸ iu ChAdOx nCoV-19 unless otherwise stated. Typically, 14 days later serum was collected and spleens harvested and cells stimulated peptides spanning the length of the S1 and S2 domains of the nCov19 spike protein.

A. End point titer (EPT) of serum IgG detected against S1 or S2 protein in BALB/c (circles) or CD1 (squares) mice.

B. Graph shows summed Spike-specific IFNγ ELISpot responses measured in BALB/c (circles) and outbred CD1 (squares) mice.

C. Graphs show the summed frequency of Spike-specific cytokine positive CD4 (left) or CD8 (right) T cells as measured by intracellular cytokine staining following stimulation of splenocytes peptides in BALB/c (circles) and CD1(squares) mice.

FIG. 8 shows antigen specific responses following ChAdOx1 nCov19 vaccination (i.e. administration of the composition of the invention to mice).

BALB/c and outbred (CD1) mice were intramuscularly administered with 10⁸ iu ChAdOx nCoV-19. 14 days later serum collected and spleens harvested and cells stimulated peptides spanning the length of S1 and S2 domains of the nCov19 spike protein.

A: IgG subclass antibodies detected against S1 (top) or S2 (bottom) protein in BALB/c (left) or CD1 (right) mice.

B. Graphs show IFNγ ELISpot responses following stimulation of splenocytes with S1 pool (black) or S2 pool (grey) in BALB/c (circle) and outbred CD1(square) mice.

C. Graphs show the frequency of cytokine positive CD4 (top) or CD8 (bottom) T cells as measured by intracellular cytokine staining following stimulation of splenocytes with S1 pool (black) or S2 pool (grey) peptides in BALB/c (circle) and CD1 (square) mice.

D. Graphs shows fold change in cytokine levels in supernatant from S1 (black) and S2 (grey) stimulated splenocytes when compared to unstimulated splenocytes for BALB/c and CD1 mice. Total IgG titres were detected against the S1 and S2 domains of the nCoV-19 spike protein in all vaccinated mice. A predominantly Th1 response was measured as assessed by subclass profiling of the IgG response (FIG. 7A & FIG. 8A). T-cell immune responses as measured by ELISpot and ICS were detected across the full length of the spike protein construct (FIG. 7B & FIG. 8B).

A predominantly Th1-type response was detected post vaccination as supported by high levels of IFN-y, TNF-α and IL-2 and low levels of IL-4 and IL-10 measured (FIG. 7C & FIGS. 8C,D). It was not expected that all mice would develop antibodies after a single shot vaccination (i.e. a single administration/single dose immunisation according to the present invention). This is evidence that the effect of the composition of the invention is surprisingly effective.

It was not expected that such a strong cellular or humoral response that was predominantly TH1 would be induced after a single-shot vaccination (i.e. a single administration/single dose immunisation according to the present invention). This is evidence that the immune responses induced by the composition of the invention / by immunisation according to the invention are surprisingly strong.

### Example 13: Demonstration in Non-Human Primates (NHP)

Acknowledgement Notice: The experimental work outlined in this example was done in collaboration with Public Health England Laboratories, Porton Down, England.

The disease induced by SARS-CoV-2 infection in Rhesus macaques (Macaca mulatto) appears similar to that in humans. Rhesus macaques were vaccinated with 2.5 x 10^10 (2.5 x 10¹⁰) viral particles of ChAdOx1 nCoV-19 (N=3 male + 3 female) by administering 100 µl via the intramuscular route to the hind leg. The control group (N=3 male + 3 female) received 100 µl of the control item (phosphate buffered saline) via the intramuscular route. All animals were challenged 4 weeks post-vaccination with SARS-CoV-2 virus. Following administration of a suitable anaesthetic, a dose of 5.0 x 10^6 (5.0 x 10⁶) pfu of SARS-CoV-2 virus in a total volume of 3 ml PBS was administered to the upper and lower respiratory tract of each animal in order to maximise the likelihood of infection. The first 2ml were delivered intratracheally followed by a 2ml flush of sterile saline, the final 1 ml was delivered intranasally divided between the nostrils. The challenge inoculum used was SARS-CoV-2 virus, VERO/hSLAM cell passage 3 (Victoria/1/2020).

Clinical observations including weight, temperature and behaviour were taken daily. Pulmonary disease burden was assessed by computed tomography (CT) scans performed 5 days after challenge and measured using a quantitative score system developed for the assessment of human COVID-19 disease. Each side of the lung was divided into a total of 12 zones as follows: Each side of the lung was divided (from top to bottom) into three zones: the upper zone (above the carina), the middle zone (from the carina to the inferior pulmonary vein), and the lower zone (below the inferior pulmonary vein). Each zone was then divided into two areas: the anterior area (the area before the vertical line of the midpoint of the diaphragm in the sagittal position) and the posterior area (the area after the vertical line of the mid-point of the diaphragm in the sagittal position). The measures used were total disease score (nodule score + ground glass opacity score + Consolidation Score) and disease distribution score (number of zones with disease). The following score system parameters were used:
nodules observed by CT scan were scored 1 4 or more
ground glass opacity (GGO) was scored according to size: <1 cm=1, 1-2 cm=2, 2-3 cm = 3, >3 cm=4
Consolidation Score: <1 cm = 1, 1-2 cm=2, 2-3 cm = 3, >3 cm=4

Plaque reducing neutralising antibody titres (PRNT) were measured in the macaque sera using the following method:

### PRNT Method

Two-fold serial dilutions starting at 1:10 of macaque sera were prepared in 96-well plates. Each serum dilution was mixed with an equal volume of virus (approximately 40-70 pfu/well) and incubated for 1 h at 37° C. Following this incubation the virus-serum mixture was transferred to Vero/E6 cell monolayers in 24-well plates. After 1-1.5 h of incubation at 37° C. the cell monolayers were overlaid with 0.5 ml of media containing 1.5% carboxymethyl cellulose. After 5 days, plates were fixed with formaldehyde. The following day, plates were washed and stained with 0.2% crystal violet solution for 5-15 minutes. Plates were washed and plaques counted. PRNT midpoint titres and 95% confidence intervals were determined by Probit analysis.

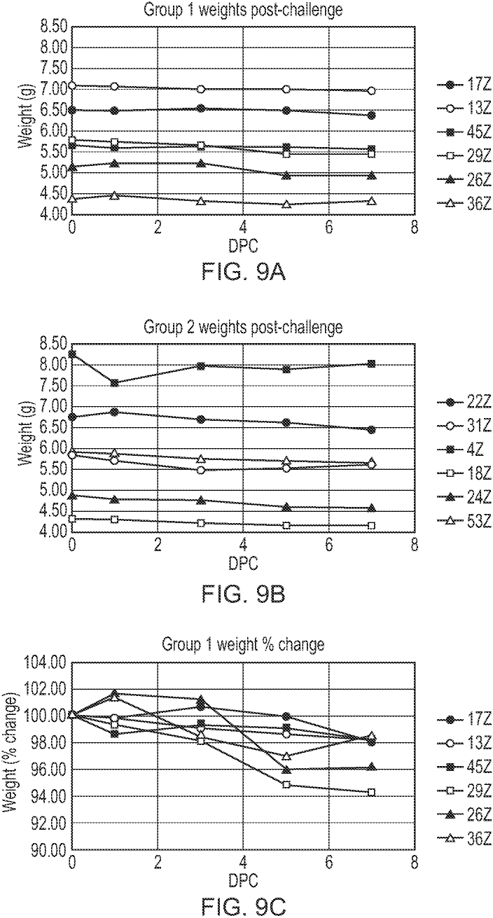

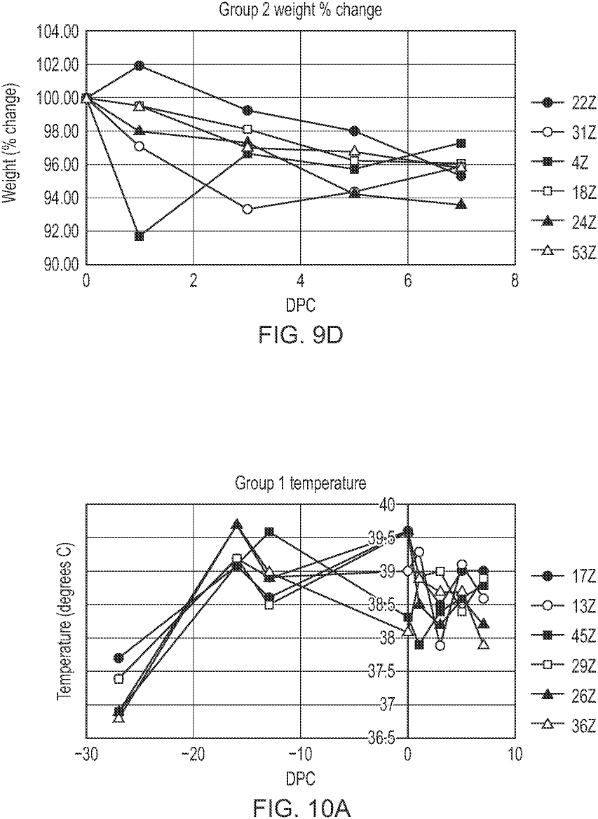

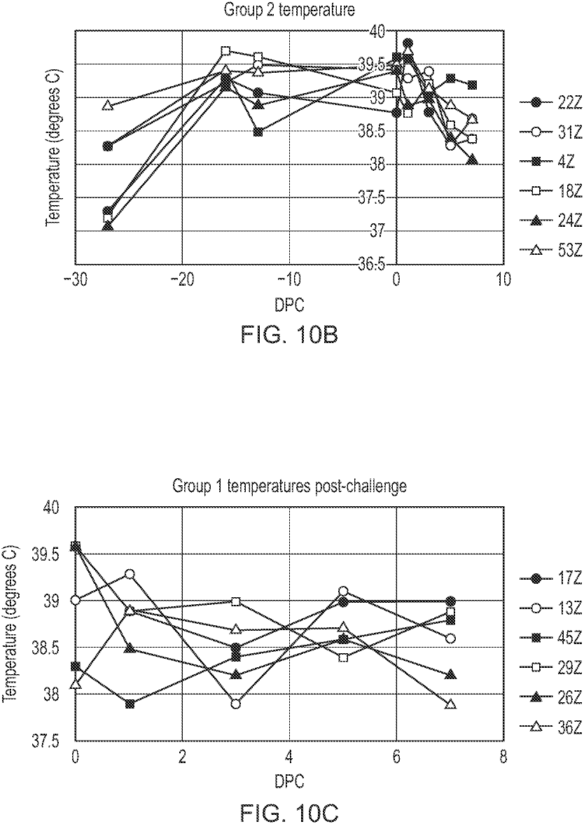

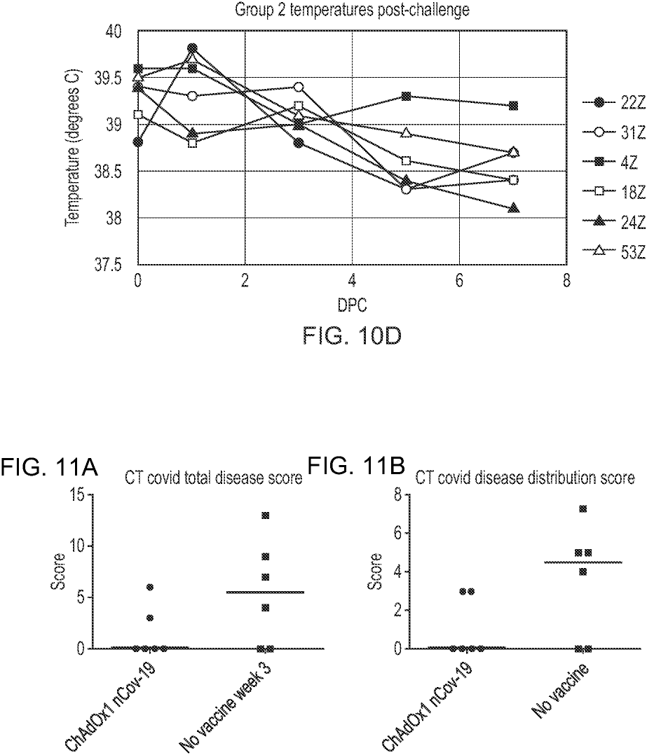

No obvious differences in weight (FIG. 9) or temperature (FIG. 10) were observed between the vaccinated and unvaccinated groups up to 7 days post challenge with the virus. Weight changes were variable between animals, some having lost weight steadily throughout the study likely as a result of the change in environment and social interactions. However, there were no clear differences between groups.

COVID-19 disease features identified from CT scans collected 5 days after challenge (Table 13 & FIG. 11) showed that fewer pulmonary abnormalities typical of the human disease were present in the animals receiving the ChAdOx1 nCoV 19 vaccine than in the unvaccinated controls. 4 out of 6 animals in the vaccinated group demonstrated no visible disease features compared with 2 out of 6 controls. Furthermore, distribution of COVID-19-induced abnormalities in those vaccinated animals that developed disease was more restricted than in the control animals, the extent of the abnormality affecting less than 25% of the lung.

We refer to FIG. 9, which shows clinical observation of weight following SARS-CoV2 virus challenge in Rhesus macaques vaccinated with ChAdOx1 nCoV-19. Animals were immunised i.m. with 2.5 x 10¹⁰ viral particles of ChAdOx1 nCoV-19 (Group 1) or phosphate buffered saline (Group 2) and challenged 4 weeks later with 5.0 x 10⁶ pfu SARS-CoV2 virus. Animals weights were measured on each day post-challenge (DPC) and plotted as absolute figures (A. & B.) and percentage weight change (C. & D.). Each line represents a single individual.

We refer to FIG. 10, which shows clinical observation of temperature following SARS-CoV2 virus challenge in Rhesus macaques vaccinated with ChAdOx1 nCoV-19. Animals were immunised i.m. with 2.5 x 10¹⁰ viral particles of ChAdOx1 nCoV-19 (Group 1) or phosphate buffered saline (Group 2) and challenged 4 weeks later with 5.0 x 10⁶ pfu SARS-CoV2 virus. Temperatures were measured pre-challenge (A. & B.) and post-challenge (C. & D.). Each line represents a single individual. DPC = days post-challenge.

Table 13 shows pulmonary disease burden measured using a quantitative score system developed for human COVID-19 in Rhesus macaques 5 days following SARS-CoV2 virus challenge. Animals were immunised i.m. with 2.5 x 10¹⁰ viral particles of ChAdOx1 nCoV-19 or phosphate buffered saline (no vaccine) and challenged 4 weeks later with 5.0 x 10⁶ pfu SARS-CoV2 virus.

TABLE 13

| Gender | Status | GGO score | Consolidation Score | Nodule Score | Total Score | Zone Score |
|---|---|---|---|---|---|---|
| male | ChAdOx1 nCov-19 | 5 | | 1 | 6 | 3 |
| male | ChAdOx1 nCov-19 | 3 | | | 3 | 3 |
| male | ChAdOx1 nCov-19 | | | | | |
| female | ChAdOx1 nCov-19 | | | | | |
| female | ChAdOx1 nCov-19 | | | | | |
| female | ChAdOx1 nCov-19 | | | | | |
| male | No vaccine | 9 | 4 | | 13 | 7 |
| male | No vaccine | | | | | |
| male | No vaccine | 4 | 2 | 1 | 7 | 5 |
| female | No vaccine | 1 | 3 | | 4 | 4 |
| female | No vaccine | | | | | |
| female | No vaccine | 1 | 8 | | 9 | 5 |

We refer to FIG. 11, which shows COVID-19 disease burden from CT images measured using a quantitative score system developed for human COVID-19 in Rhesus macaques 5 days following SARS-CoV2 virus challenge. Animals were immunised i.m. with 2.5 x 1010 viral particles of ChAdOx1 nCoV-19 or phosphate buffered saline (no vaccine) and challenged 4 weeks later with 5.0 x 10⁶ pfu SARS-CoV2 virus. Non-significant trends for differences between groups seen (Total score p= 0.1450; distribution score p = 0.0907) (Mann Whitney test)

### Significance

Given that there was no difference in total weight or temperature findings between groups it was surprising that the ChAdOx1 nCov-19 vaccinated animals had on average lower CT scores. Also surprising was that the dose of the vaccine used in this NHP study was half of the full human dose as used in the clinical trial. Typically NHP studies use the full human dose and it is surprising that protection from disease was observed following a single immunisation at the lower dose. These observations convey the effectiveness of the formulations of the invention in inducing immune responses, in particular inducing protective immune responses.

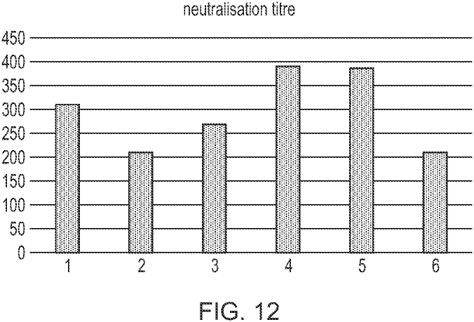

We refer to FIG. 12, showing nAb levels in 6 macaques at week 4 post vaccination. In more detail, FIG. 12 shows plaque reduction neutralising antibody titres were determined four weeks after immunising six rhesus macaques with 2.5 x 10¹⁰ viral particles of ChAdOx1 nCoV-19. All animals were strongly positive.

When convalescent human sera were tested using the same assay the PRNT results appear to come out above 320 (the top dilution). The macaque data shows levels between 200 - 380 four weeks after immunisation with ChAdOx1 nCoV19. This suggests that these are strong neutralising antibody responses in the range we expect to see in a human following natural infection with the virus. Thus these data convey that it is indeed plausible and credible that the formulations of the invention are effective in inducing the required immune responses in subjects to which the formulations are administered.

### Example 14: Evaluation of the Immunogenicity of Prime-Boost Vaccination

In this Example, the immunogenicity of one or two doses of ChAdOx1 nCoV-19 in both mice and pigs is compared. Whilst a single dose induced antigen-specific antibody and T cells responses, a booster immunisation enhanced antibody responses, particularly in pigs, with a significant increase in SARS-CoV-2 neutralising titres. Testing the immunogenicity of either one or two doses of ChAdOx1 nCoV-19 in mice and pigs, will further inform clinical development.

### Results

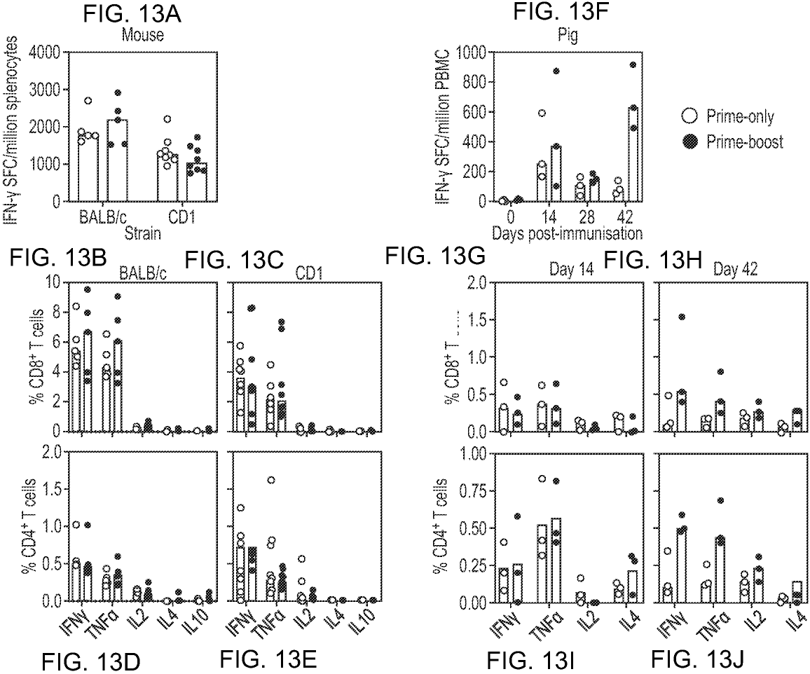

‘Prime-boost’ vaccinated inbred (BALB/c) and outbred (CD1) mice were immunised on 0 and 28 days post-vaccination (dpv), whereas, ‘prime-only’ mice received a single dose of ChAdOx1 nCoV-19 on day 28. Spleens and serum were harvested from all mice on day 49 (3 weeks after boost or prime vaccination). Analysis of SARS-CoV-2 S protein-specific murine splenocyte responses by IFN-y ELISpot assay showed no statistically significant difference between the prime-only and prime-boost vaccination regimens, in either strain of mouse (FIG. 13A). Intracellular cytokine staining (ICS) of splenocytes (FIG. 13B) showed, in both mouse strains, that the response was principally driven by CD8⁺ T cells. The predominant cytokine response of both CD8⁺ and CD4⁺ T cells was expression of IFN-γ and TNF-α, with negligible frequencies of IL-4⁺ and IL-10⁺ cells, consistent with previous data suggesting adenoviral vaccination does not induce a dominant Th2 response. There were no signficant differences in CD4⁺ and CD8⁺ T cell cytokine responses between prime-only and prime-boost mice.

Prime-only and prime-boost pigs were immunised on 0 dpv and prime-boost pigs received a second immunisation on 28 dpv. Blood samples were collected weekly until 42 dpv to analyse immune responses. IFN-γ ELISpot analysis of porcine peripheral blood mononuclear cells (PBMC) showed responses on 42 dpv (2 weeks after boost) that were significantly greater in the prime-boost pigs compared to prime-only animals (p < 0.05; FIG. 13C). The prime-boost 42 dpv responses were greater than responses observed in either group on 14 dpv, but inter-animal variation meant this did not achieve statistical significance. ICS analysis of porcine T cell reponses showed a dominance of Th1-type cytokines (similar to the murine response) but with a higher frequency of S-specific CD4⁺ T cells compared to CD8⁺ T cells (FIG. 13D). However, CD4⁺ and CD8⁺ T cell cytokine responses did not differ significantly between vaccine groups or timepoints (14 vs. 42 dpv).

In FIG. 13, SARS-CoV-2 S-specific T cell responses following ChAdOx1 nCoV-19 prime-only and prime-boost vaccination regimens in mice and pigs are shown. Inbred BALB/c (n=5) and outbred CD1 (n=8) were immunised on day 0 and 28 with ChAdOx1 nCoV19 (Prime-boost) or ChAdOx1 nCoV19 on day 28 (Prime-only); pigs (n=3) were immunised with ChAdOx1 nCoV-19 on days 0 and 28 (Prime-boost), or only on day 0 (Prime-only). To analyse SARS-CoV-2 S-specific T cell responses, all mice were sacrificed on day 49 for isolation of splenocytes and pigs were blood sampled longitudinally to isolate PBMC. Following stimulation with SARS-CoV-2 S-peptides, responses of murine splenocytes (A) and porcine PBMC (C) were assessed by IFN-γ ELISpot assays. Using flow cytometry, CD4⁺ and CD8⁺ T cell responses were characterised by assessing expression of IFN-y, TNF-α, IL-2, IL-4 and IL-10 (mice; B) and IFN-y, TNF-α, IL-2 and IL-4 (pigs; D). Each data point represents an individual mouse/pig with bars denoting the median response per group/timepoint.

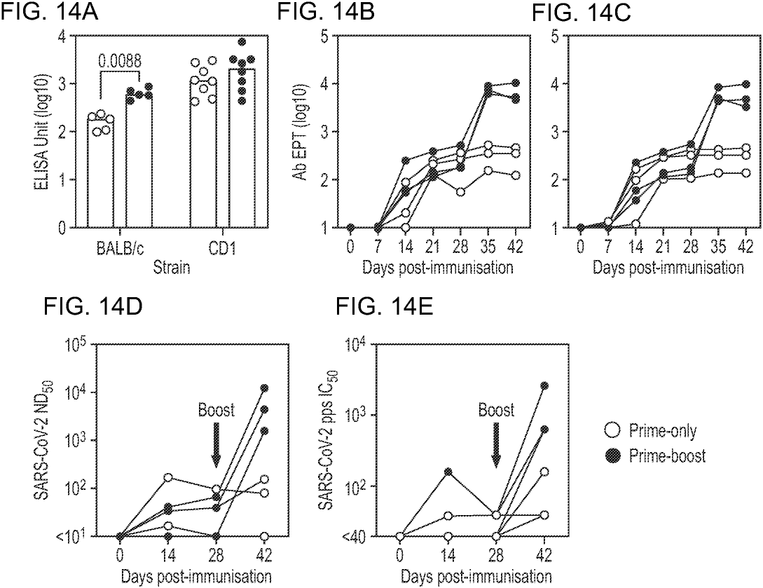

In FIG. 14 SARS-CoV-2 S protein-specific antibody responses following ChAdOx1 nCoV-19 prime-only and prime-boost vaccination regimens in mice and pigs are shown. Inbred BALB/c (n=5) and outbred CD1 (n=8) were immunised on day 0 and 28 with ChAdOx1 nCoV19 (Prime-boost) or ChAdOx1 nCoV19 on day 28 (Prime-only), whereas, pigs were immunised with ChAdOx1 nCoV-19 on days 0 and 28 (Prime-boost), or only on day 0 (Prime-only). To analyse SARS-CoV-2 S protein-specific antibodies in serum, all mice were sacrificed on day 49 and pigs were blood sampled weekly until day 42. Antibody units or end-point titres (EPT) were assessed by ELISA using recombinant SARS-CoV-2 FL-S for both mice (A) and pigs (B), and recombinant S protein RBD for pigs (C). SARS-CoV-2 neutralising antibody titres in pig sera were determined by VNT, expressed as the reciprocal of the serum dilution that neutralised virus infectivity in 50% of the wells (ND₅₀; D), and pVNT, expressed as reciprocal serum dilution to inhibit pseudovirus entry by 50% (IC₅₀; E). Each data point represents an individual mouse/pig sera with bars denoting the median titre per group.

SARS-CoV-2 S protein-specific antibody titres in serum were determined by ELISA using recombinant soluble trimeric S (FL-S) and receptor binding domain (RBD) proteins. A significant increase in FL-S binding antibody titres was observed in prime-boost BALB/c mice compared to their prime-only counterparts (p < 0.01), however, the difference between vaccine groups for CD1 mice was not significant (FIG. 14A). Antibody responses were evaluated longitudinally in pig sera by FL-S and RBD ELISA. Compared to pre-vaccination sera, significant FL-S specific antibody titres were detected in both prime-only and prime-boost groups from 21 and 14 dpv, respectively (p < 0.01; FIG. 14B). FL-S antibody titres did not differ significantly between groups until after the boost, when titres in the prime-boost pigs became significantly greater with an average increase in titres of > 1 log₁₀ (p < 0.0001). RBD-specific antibody titres showed a similar profile with significant titres in both groups from 14 dpv (p < 0.05) and a further significant increase in the prime-boost pigs from 35 dpv onwards which was greater than the prime-only pigs (p < 0.0001; FIG. 14C). SARS-CoV-2 neutralising antibody responses were assessed using a virus neutralisation test (VNT; FIG. 14D) and pseudovirus-based neutralisation test (pVNT; FIG. 14E). After the prime immunisation, SARS-CoV-2 neutralising antibody titres were detected by VNT in 14 and 28 dpv sera from ⅔ prime-boost and ⅔ prime-only pigs. Two weeks after the boost (42 dpv), neutralising antibody titres were detected and had increased in all prime-boost pigs, which were significantly greater than the earlier timepoints and the titres measured in the prime-only group (p < 0.01). In agreement with this analysis, serum assayed for neutralising antibodies using the pVNT revealed that antibody titres in 42 dpv prime-boost pig sera were significantly greater than earlier timepoints and the prime-only group (p < 0.001). Statistical analysis showed a highly significant correlation between pVNT and VNT titres (Spearman’s rank correlation r = 0.86; p < 0.0001).

### Discussion

In this Example, we utilised both a small and a large animal model to evaluate the immunogenicity of either one or two doses of a COVID-19 vaccine candidate, ChAdOx1 nCoV-19 (now known as AZD1222). Small animal models have variable success in predicting vaccine efficacy in larger animals but are an important stepping stone to facilitate prioritisation of vaccine targets. In contrast, larger animal models, such as the pig and non-human primates, have been shown to more accurately predict vaccine outcome in humans. The mouse data generated in this study suggested that the immunogenicity profile was at the upper end of a dose response curve, which may have saturated the immune response and largely obscured our ability to determine differences between prime-only or prime-boost regimens. We have developed the pig as a model for generating and understanding immune responses to vaccination against human influenza and Nipah virus. The inherent heterogeneity of an outbred large animal model is more representative of immune responses in humans. Extensive development of reagents to study immune responses in pigs in recent years has extended the usefulness and applicability of the pig as a model to study infectious disease. These data demonstrate the utility of the pig as a model for further evaluation of the immunogenicity of ChAdOx1 nCoV-19 and other COVID-19 vaccines.

We show here that T cell responses are higher in pigs that received a prime-boost vaccination when compared to prime only at day 42, whilst comparing responses 14 days after last immunisation demonstrates the prime-boost regimen trended toward a higher response. In addition, ChAdOx1 nCoV-19 immunisation induced robust Th1-like CD4⁺ and CD8⁺ T cell responses in both pigs and mice. This has important implications for COVID-19 vaccine development as virus-specific T cells are thought to play an important role in SARS-CoV-2 infection. While no correlate of protection has been defined for COVID-19, recent publications suggest that neutralising antibody titres may be correlated with protection in animal challenge models. A single dose of ChAdOx1 nCoV-19 induces antibody responses, but we demonstrate here that antibody responses are significantly enhanced after homologous boost in one mouse strain and to a greater extent in pigs. Importantly, a significant increase in mean neutralising antibody titres were measured in pigs after the booster vaccination, which may translate to enhanced protection in clinical studies.

### Methods

### Ethics Statement

Mouse and pig studies were performed in accordance with the UK Animals (Scientific Procedures) Act 1986 and with approval from the relevant local Animal Welfare and Ethical Review Body (Mice - Project License P9808B4F1, and pigs - Project License PP1804248).

### Cells and Viruses

Vero E6 cells were grown in DMEM containing sodium pyruvate and L-glutamine (Sigma-Aldrich, Poole, UK), 10% FBS (Gibco, Thermo Fisher, Loughborough, UK), 0.2% penicillin/streptomycin (10,000 U/mL; Gibco) (maintenance media) at 37° C. and 5% CO₂. SARS-CoV-2 isolate England-2 stocks were grown in Vero E6 cells using a multiplicity of infection (MOI) of 0.0001 for 3 days at 37° C. in propagation media (maintenance media containing 2% FBS). SARS-CoV-2 stocks were titrated on Vero E6 cells using MEM (Gibco), 2% FCS (Labtech, Heathfield, UK), 0.8% Avicel (FMC BioPolymer, Girvan, UK) as overlay. Plaque assays were fixed using formaldehyde (VWR, Leighton Buzzard, UK) and stained using 0.1% Toluidine Blue (Sigma-Aldrich). All work with live SARS-CoV-2 virus was performed in ACDP HG3 laboratories by trained personnel. The propagation, purification and assessment of ChAdOx1 nCoV-19 titres were as described previously.

### Recombinant SARS Co V-2 Proteins and Synthetic Peptides

A synthetic DNA, encoding the spike (S) protein receptor binding domain (RBD; amino acids 330-532) of SARS-CoV-2 (GenBank MN908947), codon optimised for expression in mammalian cells (IDT Technology) was inserted into the vector pOPINTTGneo incorporating a C-terminal His6 tag. Recombinant RBD was transiently expressed in Expi293™ (Thermo Fisher Scientific, UK) and protein purified from culture supernatants by immobilised metal affinity followed by a gel filtration in phosphate-buffered saline (PBS) pH 7.4 buffer. A soluble trimeric S (FL-S) protein construct encoding residues 1-1213 with two sets of mutations that stabilise the protein in a pre-fusion conformation (removal of a furin cleavage site and the introduction of two proline residues; K983P, V984P) was expressed as described. The endogenous viral signal peptide was retained at the N-terminus (residues 1-14), a C-terminal T4-foldon domain incorporated to promote association of monomers into trimers to reflect the native transmembrane viral protein, and a C-terminal His6 tag included for nickel-based affinity purification. Similar to recombinant RBD, FL-S was transiently expressed in Expi293™ (Thermo Fisher Scientific) and protein purified from culture supernatants by immobilised metal affinity followed by gel filtration in Tris-buffered saline (TBS) pH 7.4 buffer.

For analysis of T cell responses in pigs, overlapping 16 mer peptides offset by 4 residues based on the predicted amino acid sequence of the entire S protein from SARS-CoV-2 Wuhan-Hu-1 isolate (NCBI Reference Sequence: NC_045512.2) were designed and synthesised (Mimotopes, Melbourne, Australia) and reconstituted in sterile 40% acetonitrile (Sigma-Aldrich) at a concentration of 3 mg/mL. Three pools of synthetic peptides representing residues 1-331 (Pool 1), 332-748 (Pool 2) and 749-1273 (Pool 3) were prepared for use to stimulate T cells in IFN-γ ELISpot and intracellular cytokine staining (ICS) assays. For analysis of T cell responses in mice, overlapping 15 mer peptides offset by 11 residues were designed and synthesised (Mimotopes) and reconstituted in sterile 100% DMSO (Sigma-Aldrich) at a concentration of 100 mg/mL. Two peptide pools spanning S1 region (Pool 1: 1 to 77 and 317-321, Pool 2:78-167) and 2 peptide pools spanning S2 region (Pool 3:166 to 241, Pool 4:242 to 316) were used for stimulating splenocytes for IFN-y ELISpot analysis, and single pools of S1 (Pool 1 and Pool 2) and S2 (Pool 3 and Pool 4) were used to stimulate splenocytes for ICS.

### Immunogenicity Trials

Mice: Inbred female BALB/cOlaHsd (BALB/c) (Envigo) and outbred Crl:CD1 (CD1) (Charles River) of at least 6 weeks of age were randomly allocated into ‘prime-only’ or ‘prime-boost’ vaccination groups (BALB/c n=5 and CD1 n=8). Prime-boost mice were immunised intramuscularly with 10⁸ infectious units (IU) (6.02x10⁹ virus particles; vp) ChAdOx1 nCoV-19 and boosted intramuscularly four weeks later with 1 × 10⁸ IU ChAdOx1 nCoV-19. Prime-only mice received a single dose of 10⁸ IU ChAdOx1 nCoV-19 at the same time prime-boost mice were boosted. Spleens and serum were harvested from all animals a further 3 weeks later.

Pigs: Six 8-10-week-old, weaned, female, Large White-Landrace-Hampshire cross-bred pigs from a commercial rearing unit were randomly allocated to two treatment groups (n = 3): ‘Prime-only’ and ‘Prime-boost’. Both groups were immunised on day 0 with 1 × 10⁹ IU (5.12 × 10¹⁰ vp) ChAdOx1 nCoV-19 in 1 mL PBS by intramuscular injection (brachiocephalic muscle). ‘Prime-boost’ pigs received an identical booster immunisation on day 28. Blood samples were taken from all pigs on a weekly basis at 0, 7, 14, 21, 28, 35 and 42 dpv by venepuncture of the external jugular vein: 8 mL/pig in BD SST vacutainer tubes (Fisher Scientific) for serum collection and 40 mL/pig in BD heparin vacutainer tubes (Fisher Scientific) for peripheral blood mononuclear cell (PBMC) isolation.

### Detection of SARS-CoV-S-Specific Antibodies by ELISA

Mice: Antibodies to SARS-CoV-2 FL-S protein were determined by performing a standardised ELISA on serum collected 3-weeks after prime or prime-boost vaccination. MaxiSorp plates (Nunc) were coated with 100 ng/well FL-S protein overnight at 4° C., prior to washing in PBS/Tween (0.05% v/v) and blocking with Blocker Casein in PBS (Thermo Fisher Scientific) for 1 hour at room temperature (RT). Standard positive serum (pool of mouse serum with high endpoint titre against FL-S protein), individual mouse serum samples, negative and an internal control (diluted in casein) were incubated for 2 hours at RT. Following washing, bound antibodies were detected by addition of alkaline phosphatase-conjugated goat anti-mouse IgG (Sigma-Aldrich), diluted 1/5000 in casein, for 1 hour at RT and detection of anti-mouse IgG by the addition of pNPP substrate (Sigma-Aldrich). An arbitrary number of ELISA units were assigned to the reference pool and OD values of each dilution were fitted to a 4-parameter logistic curve using SOFTmax PRO software. ELISA units were calculated for each sample using the OD values of the sample and the parameters of the standard curve.

Pigs: Serum was isolated by centrifugation of SST tubes at 1300 × g for 10 minutes at RT and stored at -80° C. SARS-CoV-2 RBD and FL-S specific antibodies in serum were assessed as detailed previously with the exception of the following two steps. The conjugated secondary antibody was replaced with goat anti-porcine IgG HRP (Abcam, Cambridge, UK) at 1/10,000 dilution in PBS with 0.1% Tween₂₀ and 1% non-fat milk. In addition, after the last wash, a 100 µL of TMB (One Component Horse Radish Peroxidase Microwell Substrate, BioFX, Cambridge Bioscience, Cambridge, UK) was added to each well and the plates were incubated for 7 minutes at RT. A 100 µL of BioFX 450 nm Stop Reagent (Cambridge Bioscience) was then added to stop the reaction and microplates were read at 450 nm. End-point antibody titres (mean of duplicates) were calculated as follows: the log₁₀ OD was plotted against the log₁₀ sample dilution and a regression analysis of the linear part of this curve allowed calculation of the endpoint titre with an OD of twice the average OD values of 0 dpv sera.

### Assessment of SARS-CoV-2 Neutralising Antibody Responses

The ability of pig sera to neutralise SARS-CoV-2 was evaluated using virus and pseudovirus neutralisation assays. For both assays, sera were first heat-inactivated (HI) by incubation at 56° C. for 2 hours.

Virus neutralization test (VNT): Starting at a 1 in 5 dilution, two-fold serial dilutions of sera were prepared in 96 well round-bottom plates using DMEM containing 1% FBS and 1% Antibiotic-Antimycotic (Gibco) (dilution media). 75 µL of diluted pig serum was mixed with 75 µL dilution media containing approximately 64 plaque-forming units (pfu) SARS-CoV-2 for 1 hour at 37° C. Vero E6 cells were seeded in 96-well flat-bottom plates at a density of 1 × 10⁵ cells/mL in maintenance media one day prior to experimentation. Culture supernatants were replaced by 100 µL of DMEM containing 10% FCS and 1% Antibiotic-Antimycotic, before 100 µl of the virus-sera mixture was added to the Vero E6 cells and incubated for six days at 37° C. Cytopathic effect (CPE) was investigated by bright-field microscopy. Cells were further fixed and stained as described above, and CPE scored. Each individual pig serum dilution was tested in quadruplet on the same plate and no sera/SARS-CoV-2 virus and no sera/no virus controls were run concurrently on each plate in quadruplet. Wells were scored for cytopathic effect and neutralisation titres expressed as the reciprocal of the serum dilution that completely blocked CPE in 50% of the wells (ND₅₀). Researchers performing the VNTs were blinded to the identity of the samples.

Pseudovirus neutralisation test (pVNT): Lentiviral-based SARS-CoV-2 pseudoviruses were generated in HEK293T cells incubated at 37° C., 5% CO₂. Cells were seeded at a density of 7.5 x 10⁵ in 6 well dishes, before being transfected with plasmids as follows: 500 ng of SARS-CoV-2 spike, 600 ng p8.91 (encoding for HIV-1 gag-pol), 600 ng CSFLW (lentivirus backbone expressing a firefly luciferase reporter gene), in Opti-MEM (Gibco) along with 10 µL PEI (1 µg/mL) transfection reagent. A ‘no glycoprotein’ control was also set up using carrier DNA (pcDNA3.1) instead of the SARS-CoV-2 S expression plasmid. The following day, the transfection mix was replaced with 3 mL DMEM with 10% FBS (DMEM-10%) and incubated at 37° C. At both 48 and 72 hours post transfection, supernatants containing pseudotyped SARS-CoV-2 (SARS-CoV-2 pps) were harvested, pooled and centrifuged at 1,300 x g for 10 minutes at 4° C. to remove cellular debris. Target HEK293T cells, previously transfected with 500 ng of a human ACE2 expression plasmid (Addgene, Cambridge, MA, USA) were seeded at a density of 2 × 10⁴ in 100 µL DMEM-10% in a white flat-bottomed 96-well plate one day prior to harvesting of SARS-CoV-2 pps. The following day, SARS-CoV-2 pps were titrated 10-fold on target cells, with the remainder stored at -80° C. For pVNTs, pig sera were diluted 1:20 in serum-free media and 50 µL was added to a 96-well plate in quadruplicate and titrated 4-fold. A fixed titred volume of SARS-CoV-2 pps was added at a dilution equivalent to 10⁶ signal luciferase units in 50 µL DMEM-10% and incubated with sera for 1 hour at 37° C., 5% CO₂. Target cells expressing human ACE2 were then added at a density of 2 x 10⁴ in 100 µL and incubated at 37° C., 5% CO₂ for 72 hours. Firefly luciferase activity was then measured with BrightGlo luciferase reagent and a Glomax-Multi⁺ Detection System (Promega, Southampton, UK). Pseudovirus neutralization titres were expressed as the reciprocal of the serum dilution that inhibited luciferase expression by 50% (IC₅₀).

### Assessment of SARS-CoV-2 Specific T Cell Responses

Mice: Single cell suspension of mouse spleens were prepared by passing cells through 70 µm cell strainers and ACK lysis (Thermo Fisher) prior to resuspension in complete media (αMEM supplemented with 10% FCS, Pen-Step, L-Glut and 2-mercaptoethanol). For analysis of IFN-y production by ELISpot assay, splenocytes were stimulated with S peptide pools at a final concentration of 2 µg/ml on IPVH-membrane plates (Millipore) coated with 5 µg/ml anti-mouse IFN-γ (clone AN18; Mabtech). After 18-20 hours of stimulation, IFN-γ spot forming cells (SFC) were detected by staining membranes with anti-mouse IFN-y biotin mAb (1 µg/mL; clone R46A2, Mabtech) followed by streptavidin-alkaline phosphatase (1 µg/mL) and development with AP conjugate substrate kit (Bio-Rad). For analysis of intracellular cytokine production, cells were stimulated with 2 µg/mL S peptide pools, media or cell stimulation cocktail (containing PMA-Ionomycin, BioLegend), together with 1 µg/mL GolgiPlug (BD Biosciences) and 2 µL/mL CD107a-Alexa647 for 6 hours in a 96-well U-bottom plate, prior to placing at 4° C. overnight. Following surface staining with CD4-BUV496, CD8-PerCP-Cy5.5, CD62L-BV711, CD127-BV650, CD44-APC-Cy7 and LIVE/DEAD Aqua (Thermo Fisher), cells were fixed with 10% neutral buffered formalin (containing 4% paraformaldehyde) and stained intracellularly with TNF-α-AF488, IL-2-PE-Cy7, IL-4-BV605, IL-10-PE and IFN-γ-e450 diluted in Perm-Wash buffer (BD Biosciences). Sample acquisition was performed on a Fortessa (BD) and data analysed in FlowJo v9 (TreeStar). An acquisition threshold was set at a minimum of 5000 events in the live CD3⁺ gate. Antigen-specific T cells were identified by gating on LIVE/DEAD negative, doublet negative (FSC-H vs FSC-A), size (FSC-H vs SSC), CD3⁺, CD4⁺ or CD8⁺ cells and cytokine positive. Total SARS-CoV-2 S specific cytokine responses are presented after subtraction of the background response detected in the media stimulated control spleen sample of each mouse, prior to summing together the frequency of S1 and S2 specific cells.

Pigs: PBMCs were isolated from heparinised blood by density gradient centrifugation and cryopreserved in cold 10% DMSO (Sigma-Aldrich) in HI FBS. Resuscitated PBMC were suspended in RPMI 1640 medium, GlutaMAX supplement, HEPES (Gibco) supplemented with 10 % HI FBS (New Zealand origin, Life Science Production, Bedford, UK), 1% Penicillin-Streptomycin and 0.1% 2-mercaptoethanol (50 mM; Gibco) (cRPMI). To determine the frequency of SARS-CoV-2 S specific IFN-y producing cells, an ELISpot assay was performed on PBMC from 0, 14, 28 and 42 dpv. Multiscreen 96-well plates (MAHAS4510; Millipore, Fisher Scientific) were pre-coated with 1 µg/mL anti-porcine IFN-y mAb (clone P2G10, BD Biosciences) and incubated overnight at 4° C. After washing and blocking with cRPMI, PBMCs were plated at 5 × 10⁵ cells/well in cRPMI in a volume of 50 uL/well. PBMCs were stimulated in triplicate wells with the SARS-CoV-2 S peptide pools at a final concentration of 1 µg/mL/peptide. cRPMI alone was used in triplicate wells as a negative control. After 18 hours incubation at 37° C. with 5% CO₂, plates were developed as described previously. The numbers of specific IFN-y secreting cells were determined using an ImmunoSpot® S6 Analyzer (Cellular Technology, Cleveland, USA). For each animal, the mean ‘cRPMI only’ data was subtracted from the S peptide pool 1, 2 and 3 data which were then summed and expressed as the medium-corrected number of antigen-specific IFN-y secreting cells per 1 x 10⁶ PBMC. To assess intracellular cytokine expression PBMC from 14 and 42 dpv were suspended in cRPMI at a density of 2 × 10⁷ cells/mL and added to 50 µL/well to 96-wellround bottom plates. PBMCs were stimulated in triplicate wells with the SARS-CoV-2 S peptide pools (1 µg/mL/peptide). Unstimulated cells in triplicate wells were used as a negative control. After 14 hours incubation at 37° C., 5% CO₂, cytokine secretion was blocked by addition 1:1,000 BD GolgiPlug (BD Biosciences) and cells were further incubated for 6 hours. PBMC were washed in PBS and surface labelled with Zombie NIR fixable viability stain (BioLegend), CD4-PerCP-Cy5.5 mAb (clone 74-12-4, BD Bioscience) and CD8β-FITC mAb (clone PPT23, Bio-Rad Antibodies). Following fixation (Fixation Buffer, BioLegend) and permeabilization (Permeabilization Wash Buffer, BioLegend), cells were stained with: IFN-γ-AF647 mAb (clone CC302, Bio-Rad Antibodies, Kidlington, UK), TNF-α-BV421 mAb (clone Mab11, BioLegend), IL-2 mAb (clone A150D 3F1 2H2, Invitrogen, Thermo Fisher Scientific) and IL-4 BV605 mAb (clone MP4-25D2, BioLegend) followed by staining with anti-mouse IgG2a-PE-Cy7 (clone RMG2a-62, BioLegend). Cells were analysed using a BD LSRFortessa flow cytometer and FlowJo X software. Total SARS-CoV-2 S specific cytokine positive responses are presented after subtraction of the background response detected in the media stimulated control PBMC sample of each pig, prior to summing together the frequency of S-peptide pools 1-3 specific cells.

### References to Example 14

1 Zhu, F.-C. et al. Safety, tolerability, and immunogenicity of a recombinant adenovirus type-5 vectored COVID-19 vaccine: a dose-escalation, open-label, non-randomised, first-in-human trial. The Lancet, doi:https://doi.org/10.1016/S0140-673 6(20)3 1208-3 (2020).

2 van Doremalen, N. et al. A single-dose ChAdOx1-vectored vaccine provides complete protection against Nipah Bangladesh and Malaysia in Syrian golden hamsters. PLoS Negl Trop Dis 13, e0007462, doi:10.1371/journal.pntd.0007462 (2019).

3 Folegatti, P. M. et al. Safety and immunogenicity of a candidate Middle East respiratory syndrome coronavirus viral-vectored vaccine: a dose-escalation, open-label, non-randomised, uncontrolled, phase 1 trial. The Lancet. Infectious diseases, doi:10.1016/s1473-3099(20)30160-2 (2020).

4 Munster, V. J. et al. Protective efficacy of a novel simian adenovirus vaccine against lethal MERS-CoV challenge in a transgenic human DPP4 mouse model. NPJ Vaccines 2, 28-28, doi:10.1038/s41541-017-0029-1 (2017).

5 van Doremalen, N. et al. A single dose of ChAdOx1 MERS provides broad protective immunity against a variety of MERS-CoV strains. bioRxiv, 2020.2004.2013.036293, doi:10.1101/2020.04.13.036293 (2020).

6 van Doremalen, N. et al. ChAdOx1 nCoV-19 vaccination prevents SARS-CoV-2 pneumonia in rhesus macaques. bioRxiv, 2020.2005.2013.093195, doi:10.1101/2020.05.13.093195 (2020).

7 van Doremalen, N. et al. A single dose of ChAdOx1 MERS provides protective immunity in rhesus macaques. Science Advances, eaba8399, doi:10.1126/sciadv.aba8399 (2020).

8 Suleman, M. et al. Antigen encoded by vaccine vectors derived from human adenovirus serotype 5 is preferentially presented to CD8+ T lymphocytes by the CD8α+ dendritic cell subset. Vaccine 29, 5892-5903, doi:10.1016/j.vaccine.2011.06.071 (2011).

9 Zhang, Y. et al. Immunization with an adenovirus-vectored TB vaccine containing Ag85A-Mtb32 effectively alleviates allergic asthma. J Mol Med (Berl) 96, 249-263, doi:10.1007/s00109-017-1614-5 (2018).

10 Meurens, F., Summerfield, A., Nauwynck, H., Saif, L. & Gerdts, V. The pig: a model for human infectious diseases. Trends in microbiology 20, 50-57, doi:10.1016/j.tim.2011.11.002 (2012).

11 Gerdts, V. et al. Large Animal Models for Vaccine Development and Testing. ILAR Journal 56, 53-62, doi:10.1093/ilar/ilv009 (2015).

12 Rivera-Hernandez, T. et al. The contribution of non-human primate models to the development of human vaccines. Discov Med 18, 313-322 (2014).

13 Holzer, B. et al. Comparison of Heterosubtypic Protection in Ferrets and Pigs Induced by a Single-Cycle Influenza Vaccine. Journal of immunology (Baltimore, Md. : 1950) 200, 4068-4077, doi:10.4049/jimmunol.1800142 (2018).

14 Holzer, B. et al. Immunogenicity and Protective Efficacy of Seasonal Human Live Attenuated Cold-Adapted Influenza Virus Vaccine in Pigs. Front Immunol 10, 2625, doi:10.3389/fimmu.2019.02625 (2019).

15 Morgan, S. B. et al. Aerosol Delivery of a Candidate Universal Influenza Vaccine Reduces Viral Load in Pigs Challenged with Pandemic H1N1 Virus. Journal of immunology (Baltimore, Md. : 1950) 196, 5014-5023, doi:10.4049/jimmunol.1502632 (2016).

16 McLean, R. K. & Graham, S. P. Vaccine Development for Nipah Virus Infection in Pigs. Front Vet Sci 6, 16, doi:10.3389/fvets.2019.00016 (2019).

17 Pedrera, M. et al. Bovine Herpesvirus-4-Vectored Delivery of Nipah Virus Glycoproteins Enhances T Cell Immunogenicity in Pigs. Vaccines (Basel) 8, doi:10.3390/vaccines8010115 (2020).

18 Grifoni, A. et al. Targets of T Cell Responses to SARS-CoV-2 Coronavirus in Humans with COVID-19 Disease and Unexposed Individuals. Cell, doi:10.1016/j.cell.2020.05.015 (2020).

19 Zheng, H. Y. et al. Elevated exhaustion levels and reduced functional diversity of T cells in peripheral blood may predict severe progression in COVID-19 patients. Cell Mol Immunol 17, 541-543, doi:10.1038/s41423-020-0401-3 (2020).

20 Chen, G. et al. Clinical and immunological features of severe and moderate coronavirus disease 2019. J Clin Invest 130, 2620-2629, doi:10.1172/jci137244 (2020).

21 Xiong, Y. et al. Transcriptomic characteristics of bronchoalveolar lavage fluid and peripheral blood mononuclear cells in COVID-19 patients. Emerg Microbes Infect 9, 761-770, doi:10.1080/22221751.2020.1747363 (2020).

22 Braun, J. et al. Presence of SARS-CoV-2 reactive T cells in COVID-19 patients and healthy donors. medRxiv, 2020.2004.2017.20061440, doi:10.1101/2020.04.17.20061440 (2020).

23 Chandrashekar, A. et al. SARS-CoV-2 infection protects against rechallenge in rhesus macaques. Science, doi:10.1126/science.abc4776 (2020).

24 Yu, J. et al. DNA vaccine protection against SARS-CoV-2 in rhesus macaques. Science, eabc6284, doi:10.1126/science.abc6284 (2020).

25 Amanat, F. et al. A serological assay to detect SARS-CoV-2 seroconversion in humans. Nature Medicine, doi:10.1038/s41591-020-0913-5 (2020).

### Data Analysis

GraphPad Prism 8.1.2 (GraphPad Software, San Diego, USA) was used for graphical and statistical analysis of data sets. ANOVA or a mixed-effects model were conducted to compare responses over time and between vaccine groups at different time points post-vaccination as detailed in the Results. Neutralising antibody titre data were log transformed before analysis. Neutralising antibody titre data generated by the VNT and pVNT assays were compared using Spearman nonparametric correlation. p-values < 0.05 were considered statistically significant.

### Example 15: Demonstration in Humans

In this example we describe Safety and immunogenicity of the ChAdOx1 nCoV-19 vaccine against SARS-CoV-2: a Phase I/II randomized control trial.

We refer to Example 1 and Example 2 where such trials are disclosed; we refer to Example 4 where such trials are disclosed in more detail; we refer to Example 8 where trial outlines are disclosed in even more detail.

More importantly we specifically refer to Example 11 above which discloses in comprehensive detail the human clinical trial which is discussed below. It may aid understanding to read this example in conjunction with Example 11. The skilled reader will note that there is a difference in the numbers of volunteers in the discussion below compared to the discussion in Example 11. For all other substantive details, Example 11 may be consulted as necessary.

### Background

We conducted a phase ½ single-blind randomised controlled trial of ChAdOx1 nCoV-19 compared with MenACWY, as control vaccine, in healthy adults in the United Kingdom.

In this example, we describe the immunogenicity, reactogenicity, and safety of vaccination with 5×10¹⁰ vp of ChAdOx1 nCoV-19 after 1 month.

### Methods

### Vaccine

The ChAdOx1 nCoV-19 vaccine consists of the replication-deficient simian adenovirus vector ChAdOx1, containing the full-length structural surface glycoprotein (spike protein) of SARS-CoV-2 (nCoV-19), with a tissue plasminogen activator (tPA) leader sequence. ChAdOx1 nCoV-19 expresses a codon-optimised coding sequence for the spike protein from genome sequence accession GenBank: MN908947. The recombinant adenovirus was produced as previously described.⁹ The vaccine was manufactured according to current Good Manufacturing Practice by the Clinical BioManufacturing Facility (University of Oxford, Oxford, UK).

A licensed meningococcal group A, C, W-135, and Y conjugate vaccine (MenACWY, Nimenrix, Pfizer, UK) was used as the active comparator in order to maintain blinding of participants who experienced local or systemic reactions.

### Study Design and Participants

This is an ongoing phase ½, participant-blinded, multi-centre, randomised controlled trial. The study is being conducted at 5 centres in the UK (Centre for Clinical Vaccinology and Tropical Medicine, University of Oxford; University Hospital Southampton NHS Foundation Trust, Southampton; Clinical Research Facility at Imperial College London; St Georges University of London and University Hospital NHS Foundation Trust; and University Hospitals Bristol and Weston NHS Foundation Trust.). Healthy adult participants aged 18-55 years were recruited through local advertisements. All participants underwent a screening visit where a full medical history and examination was taken in addition to blood and urine tests (HIV, hepatitis B and C serology, full blood count, kidney and liver function tests, and urinary screen for blood, protein and glucose and a pregnancy test done in women of childbearing potential). Volunteers with a history of laboratory confirmed COVID-19, those at higher risk for COVID-19 exposure pre-enrolment and those with a new onset of fever, cough, shortness of breath and anosmia/ageusia since February 2020 were excluded from the study. Full details of the eligibility criteria are described in the trial protocol provided in the supplementary material.

Written informed consent was obtained from all participants, and the trial is being conducted in accordance with the principles of the Declaration of Helsinki and Good Clinical Practice. This study was approved in the UK by the Medicines and Healthcare Products Regulatory Agency (reference 21584/0424/001-0001) and the South Central Berkshire Research Ethics Committee (reference 20/SC/0145). Vaccine use was authorised by the Genetically Modified Organisms Safety Committee of the Oxford University Hospitals National Health Service Trust (reference number GM462.20.129).

### Randomisation and Masking

Participants were randomised 1:1 to receive the ChAdOx1 nCoV-19 at 5 × 10¹⁰ viral particles or MenACWY vaccines. Randomisation lists, using block randomisation stratified by study group and study site were generated by the study statistician. Block sizes of 2, 4 and 6 were chosen to align with the study group sizes and the number of doses available per vial, and varied across study groups. Computer randomisation was done with full allocation concealment within the secure web platform used for the study eCRF (REDCap 9.5.22 - © 2020 Vanderbilt University). The trial staff administering the vaccine prepared vaccines out of sight of the participant and syringes were covered with an opaque material until ready for administration to ensure blinding of participants.

### Procedures

Both vaccines were administered as a single intramuscular injection into the deltoid. A staggered-enrolment approach was used and interim safety reviews with the independent Data and Safety Monitoring Board (DSMB) were conducted before proceeding with vaccinations in larger numbers of volunteers. Volunteers were considered enrolled into the trial at the point of vaccination. Ten participants were enrolled in a non-randomised prime-boost group.

Participants had blood samples drawn and clinical assessments for safety as well as immunology at day 0, 28 and will also be followed at day 184 and 364. In addition, participants enrolled in the phase 1 component of the study and in the prime-boost group, had visits 3, 7, and 14 days after each vaccination. A later amendment to the protocol provided for additional testing of booster vaccinations in a subset of participants, the results of which are not yet available and are not included in this report.

A non-randomised subgroup of participants received 1 g prophylactic paracetamol prior to vaccination and advised to continue with 1 g every 6 hours for 24 hours to reduce vaccine-associated reactions.

Participants were observed in the clinic for 1 h after the vaccination procedure and were asked to record any adverse events (AEs) using electronic diaries during the 28-day follow-up period. Expected and protocol defined local site reactions (injection site pain, tenderness, warmth, redness, swelling, induration and pruritus) and systemic symptoms (malaise, myalgia, arthralgia, fatigue, nausea, headache, feverishness, and objective fever defined as an oral temperature of 38° C. or higher) were recorded for 7 days. All other events were recorded for 28 days, and serious adverse events are recorded throughout the follow-up period.

Severity of AEs are graded with the following criteria: mild (transient or mild discomfort <48 hours, no interference with activity, no medical intervention/therapy required), moderate (mild to moderate limitation in activity, some assistance may be needed; no or minimal medical intervention/therapy required), severe (marked limitation in activity, some assistance usually required; medical intervention/therapy required), and potentially life-threatening (requires assessment in A&E or hospitalisation). Unsolicited AEs are reviewed for causality by two clinicians blinded to group allocation, and events considered to be possibly, probably, or definitely related to the study vaccines were reported. Laboratory AEs were graded by use of site-specific toxicity tables, which were adapted from the US Food and Drug Administration toxicity grading scale.

### Immunogenicity Measures

Humoral responses to vaccination were assessed using a total IgG ELISA against trimeric SARS CoV-2 spike protein, two live SARS Cov2 neutralisation assays and a pseudovirus neutralisation assay. Details are as follows:

### ELISA for Detection of Sera SARS-CoV-2 Antigen Specific Total IgG

Total anti-SARS CoV-2 antibodies were determined using an in-house indirect ELISA that uses a standard curve derived from a pool of SARS-COV-2 convalescent plasma samples on every plate. Briefly, plates were coated with 2 µg/mL of full-length trimerised SARS-CoV-2 spike glycoprotein (produced in-house) and stored at 4° C. overnight for at least 16 h. After coating, plates were washed with PBS/0.05%Tween and blocked with Casein. Thawed samples diluted in casein were plated in triplicate alongside two internal positive controls (Controls 1 and 2) to measure plate to plate variation. Control 1 was a dilution of convalescent plasma sample and Control 2 was a research reagent for anti-SARS-CoV-2 Ab (code 20/130 supplied by National

Institute for Biological Standards and Control (NIBSC)). The standard pool was used in a two-fold serial dilution to produce ten standard points that were assigned arbitrary ELISA units (EUs). Goat anti-human IgG (y-chain specific) conjugated to alkaline phosphatase was used as secondary antibody and plates were developed by adding 4-nitrophenyl phosphate in diethanolamine substrate buffer. Standardised EUs were determined from a single dilution of each sample against the standard curve which was plotted using the 4-Parameter logistic model (Gen5 v3.09, Biotek). Each assay plate consisted of samples and controls plated in triplicate, with ten standard points in duplicate and four blank wells.

### Marburg SARS-CoV-2 Virus Neutralization

SARS-CoV-2 neutralizing activity of human sera was investigated based on previously published protocols for MERS-CoV (PMID: 32325038, 32325037). Briefly, samples were heat-inactivated for 30 min at 56° C. and serially diluted in 96-well plates starting from a dilution of 1:8. Samples were incubated for 1 h at 37° C. together with 100 50% tissue culture infective doses (TCID50) SARS-CoV-2 (BavPat1/2020 isolate, European Virus Archive Global # 026V-03883). Cytopathic effect (CPE) on VeroE6 cells (Vero C1008, ATCC, Cat#CRL-1586, RRID: CVCL_0574) was analyzed 4 days post-infection. Neutralization was defined as absence of CPE compared to virus controls. For each test, a positive control (neutralizing COVID-19 patient plasma) was used in duplicates as an inter-assay neutralization standard. (Kreer et al 2020; biorxiv “Longitudinal isolation of potent near-germline SARS-CoV-2-neutralizing antibodies from COVID-19 patients” d.o.i: 10.1101/2020.06.12.146290v1)

### Monogram Pseudotype Neutralisation Assay

A lentivirus-based SARSCoV-2 pseudovirus particle was generated expressing spike protein on the surface. The PS CoV nAb assay is based on previously described methodologies using HIV-1 pseudovirions (Petropoulos et al., AAC 2000, Richman et al, PNAS 2003, Whitcomb et al., 2007). Briefly, serum samples were heat inactivated at 56° C. for one hour and diluted 1:40 with SARS CoV-2 negative human serum. Neutralizing antibody (Nab) titres were determined by endpoint three-fold serial dilutions of pre-mixed test samples mixed with 10⁵ relative light units (RLU) of SARS-CoV2 pseudotyped virus incubated at 37° C. for one hour and then mixed with 10⁷ HEK 293 ACE2-transfected cells per well. Separately, irrelevant pseudotyped control virus, were also mixed with test samples. Plates were incubated for 60-80 hours at 37° C. and then assayed for luciferase expression. Neutralization titres are reported as the reciprocal of the serum dilution conferring 50% inhibition (ID50) of pseudovirus infection. %Inhibition = 100% -(((RLU(Vector+Sample+Diluent) - RLU(Background))/(RLU(Vector+Diluent) -RLU(Background))) x 100%). SARS CoV-2 nAb Assay Positive and Negative Control Sera are included on each 96-well assay plate (1 positive control, 1 negative control, 6 patient specimens).

### Public Health England Plaque Reduction Neutralisation Test

Neutralising virus titres were measured in heat-inactivated (56° C. for 30 min) serum samples. SARS-CoV-2 was diluted to a concentration of 933 pfu/ml (70 pfu/75 µl) and mixed 50:50 in 1% FCS/MEM with doubling serum dilutions from 1:20 to 1:640 in a 96-well V-bottomed plate. The plate was incubated at 37° C. in a humidified box for 1 hour to allow the antibody in the serum samples to bind the virus. The virus and serum dilutions were transferred into the wells of a washed plaque assay 24-well plate, allowed to adsorb at 37° C. for an hour, and overlaid with plaque assay overlay media. After 5 days incubation at 37° C. in a humified box, the plates were fixed, stained and plaques counted. Median neutralising titres (ND₅₀) were determined using the Spearman- Karber formula relative to virus only control wells.

### Public Health England Microneutralisation Assay

The principle of the MNA is similar to the PRNT. Virus susceptible monolayers (Vero/E6 Cells) in 96 well plates were exposed to the serum/virus mixture prepared as for PRNT. Plates were incubated in a sealed humified box for 1 hour before removal of the virus inoculum and replacement with overlay (1% w/v CMC in complete media). The box was resealed and incubated for 24 hours prior to fixing for formaldehyde. Microplaques were visualised using a SARS-CoV-2 antibody specific for the SARS-CoV-2 RBD Spike protein and a rabbit HRP conjugate, infected foci were visualised using TrueBlue™ substrate. Stained microplaques were counted using ImmunoSpot® S6 Ultra-V Analyzer and resulting counts analysed in SoftMax Pro v7.0 software.

### Multiplexed Immunoassay

A multiplexed immunoassay was developed to measure the antigen-specific response to ChAdOx1 nCoV-19 vaccination and/or natural SARS-CoV-2 infection (MesoScaleDiscovery, Rockville, MD). A 10-Spot Custom SARS-CoV2 Serology SECTOR® plate was coated with SARS-CoV2 Antigens Spike, N, and RBD, produced by MesoScaleDiscovery. Pooled human serum were developed for internal quality controls and as reference standard reagents. To assess anti- Spike and anti-RBD, IgG, antigens were coated onto plates at 200 to 400 µg/mL in PBS. Equilibrated plates were blocked with Blocker A for 1 hr and washed 3 times prior to the addition of reference standard, controls and samples. After incubation for 2 hours, the plates were washed 3 times and IgG (SULFO-TAG Conjugated Anti-Hu/IGG, clone 2A11) detection antibodies were added at a concentration of 200 µg/mL. Plates were incubated for 1 hour, washed 3 times, and read in MSD Read Buffer T.

### Ex Vivo IFNγ ELISPOT to Enumerate Antigen-Specific T Cells

ELISpot assays were performed using freshly isolated peripheral blood mononuclear cells (PBMCs) to determine responses to the SARS-CoV-2 spike vaccine antigen at days 0 (before vaccination), 7 14, 28 and 56, and also at D35 and 42 in participants that received two doses. Assays were performed using Multiscreen IP ELISpot plates (Merck Millipore, Watford, UK) coated with 10 µg/mL human anti-IFN-y antibody and developed using SA-ALP antibody conjugate kits (Mabtech, Stockholm, Sweden) and BCIP NBT-plus chromogenic substrate (Moss

Inc., Pasadena, MA, USA). PBMC were separated from whole blood with lithium heparin by density centrifugation within four hours of venepuncture. Cells were incubated for 18-20 hours in RPMI (Sigma) containing 1000 units/mL penicillin, 1 mg/mL streptomycin and 10% heat-inactivated, sterile-filtered foetal calf serum, previously screened for low reactivity (Labtech International, East Sussex, UK) with a final concentration of 10 µg/ml of pooled peptide. A total of 253 synthetic peptides (15 mers overlapping by 10 amino acids) spanning the entire vaccine insert, including the tPA leader sequence were used to stimulate PBMC (Pro-Immune, Oxford UK). Peptides were pooled into 12 pools for the SARS-CoV-2 spike protein containing 18 to 24 peptides, plus a single pool of 5 peptides for the tPA leader. Peptide sequences and pooling are summarised in Supplementary Table S4. Peptides were tested in triplicate, with 2.5 × 10⁵ PBMC added to each well of the ELISpot plate in a final volume of 100 µL. Results are expressed as spot forming cells (SFC) per million PBMCs, calculated by subtracting the mean negative control response from the mean of each peptide pool response and then summing the response for the 13 peptide pools. Staphylococcal enterotoxin B (0.02 µg/mL) and phytohaemagglutinin-L (10 µg/ mL) were pooled and used as a positive control. Plates were counted using an AID automated ELISpot counter (AID Diagnostika GmbH, algorithm C, Strassberg, Germany) using identical settings for all plates, and counts were adjusted only to remove artefacts. A quality control process was applied where plates were excluded if responses were >80 SFC/million PBMC in the negative control (PBMC without antigen) or <800 SFC/million PBMC in the positive control wells. Responses to the negative control were low, with a median of XX SFC (interquartile range (IQR) XX-XX).

### Outcomes

The co-primary objectives are to assess efficacy against symptomatic virologically confirmed COVID-19 disease and occurrence of serious adverse events. Secondary outcomes include safety, reactogenicity, and immunogenicity profiles of ChAdOx1 nCoV-19, and efficacy against hospital attended COVID-19 disease, death and seroconversion against non-spike proteins. Preliminary results for secondary endpoints are reported here: occurrence of local and systemic reactogenicity signs and symptoms for 7 days after vaccination; occurrence of unsolicited adverse events for 28 days after vaccination; change from day 0 (baseline) to day 28 for safety laboratory measures; occurrence of serious adverse events; cellular and humoral immunogenicity of ChAdOx1 nCoV-19.

### Study of Convalescent Sera From COVID-19 Patients

Samples from individuals ≥18 years of age with PCR positive SARS-CoV-2 infection were obtained from patient cohorts admitted to hospital or surveillance on healthcare workers. (Gastrointestinal illness in Oxford: COVID sub study [Sheffield REC, reference: 16/YH/0247], ISARIC/WHO Clinical Characterisation Protocol for Severe Emerging Infections [Oxford REC C, reference 13/SC/0149], Sepsis Immunomics project [Oxford REC C, reference:19/SC/0296])

### Statistical Analysis

Safety endpoints are described as frequencies and percentages with 95% binomial exact confidence intervals (CI). Medians and interquartile ranges are presented for immunological endpoints and analyses are considered descriptive only as the full set of samples have not yet been analysed on all platforms and therefore results reported here are preliminary.

Statistical analyses were conducted using SAS version 9.4 and R version 3.6.1 or later. The sample size for the study was determined by the number of doses of vaccine that were available for use after the initial clinical manufacturing process. Sample sizes for efficacy are based on the number of primary outcome events that accrue and are presented in the protocol attached as a supplementary file. Efficacy analyses have not been conducted and are not included in this report. An independent Data and Safety Monitoring Board provided safety oversight (see Supplementary File). This study is registered with ClinicalTrials.gov, NCT04324606 and with ISRCTN, number 15281137

### Study

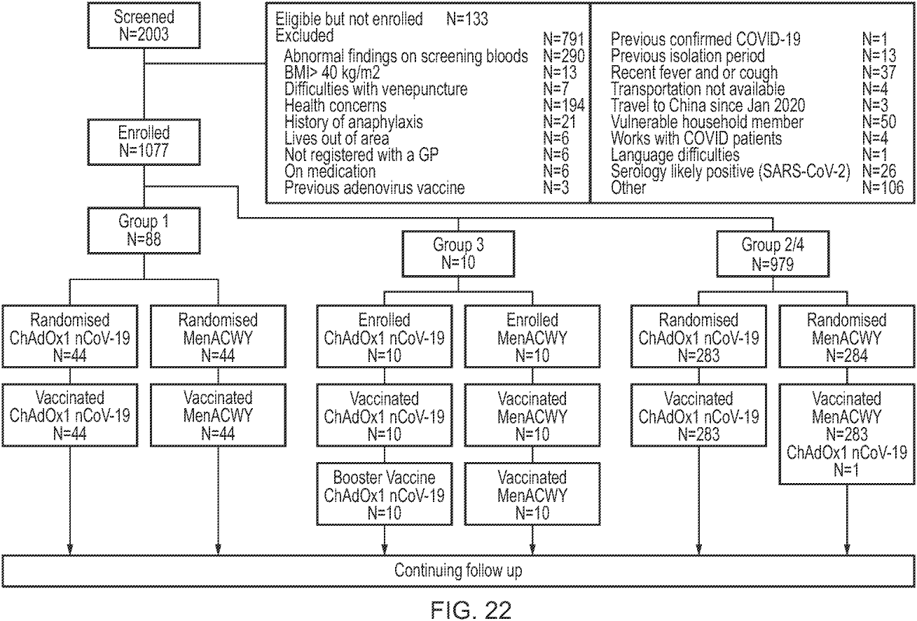

Between April 23^{rd} and May 21st 2020, 1077 participants were enrolled into the study and vaccinated with either ChAdOx1 nCoV-19 or MenACWY control vaccine. (FIG. 22).

The median age of participants was 35 years (IQR 28, 44 years), 50% of participants were female and 91% of participants were white (see Table below). Baseline characteristics were similar between randomised groups (see Table below).

Table: Baseline characteristics of randomised participants

| | ChAdOx1 N=543 | MenACWY N=534 |
|---|---|---|
| Female sex (n %) | 265 (49%) | 271 (51%) |
| Age, years, median [IQR] | 34 [28, 43] | 36 [28, 45] |
| Non-smoker | 495 (91%) | 485 (91%) |
| Non-drinker | 89 (16%) | 60 (11%) |
| BMI, median [IQR] | 24 [22, 27] | 24 [22, 27] |
| Ethnicity | | |
| Arab | 2 (<1%) | 2 (<1%) |
| Black | 4 (1%) | 2 (<1%) |
| E.Asian | 5 (1%) | 4 (1%) |
| S. Asian | 15 (3%) | 20 (4%) |
| Mixed | 12 (2%) | 9 (2%) |
| Other | 11 (2%) | 10 (2%) |
| Not given | 1 (<1%) | 1 (<1%) |
| White | 493 (91%) | 486 (91%) |

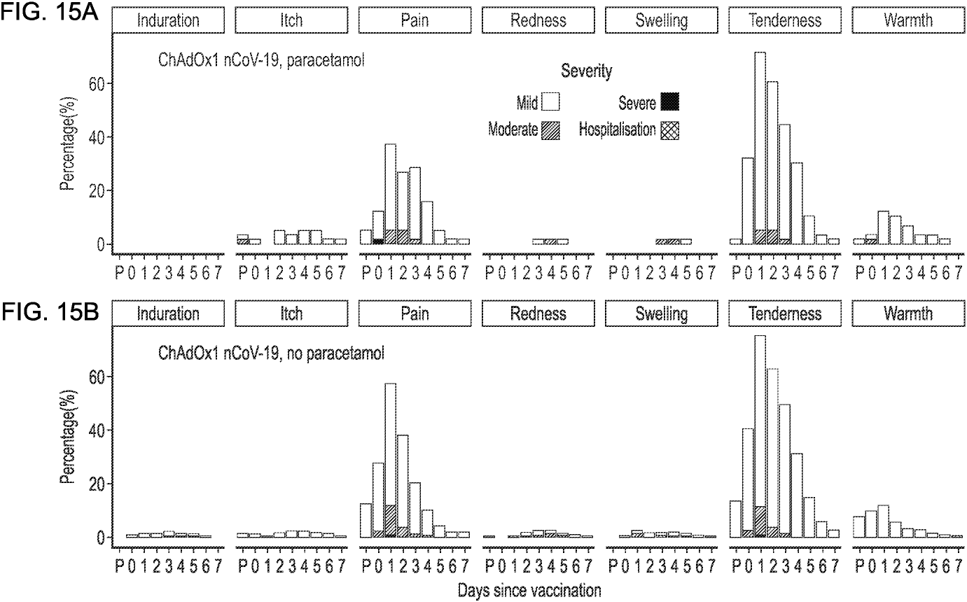

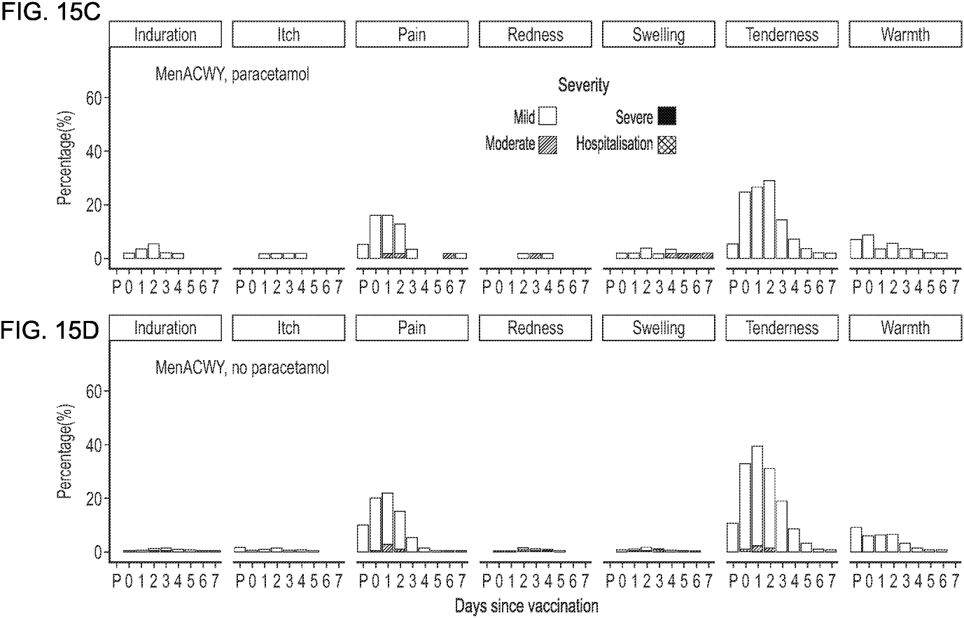

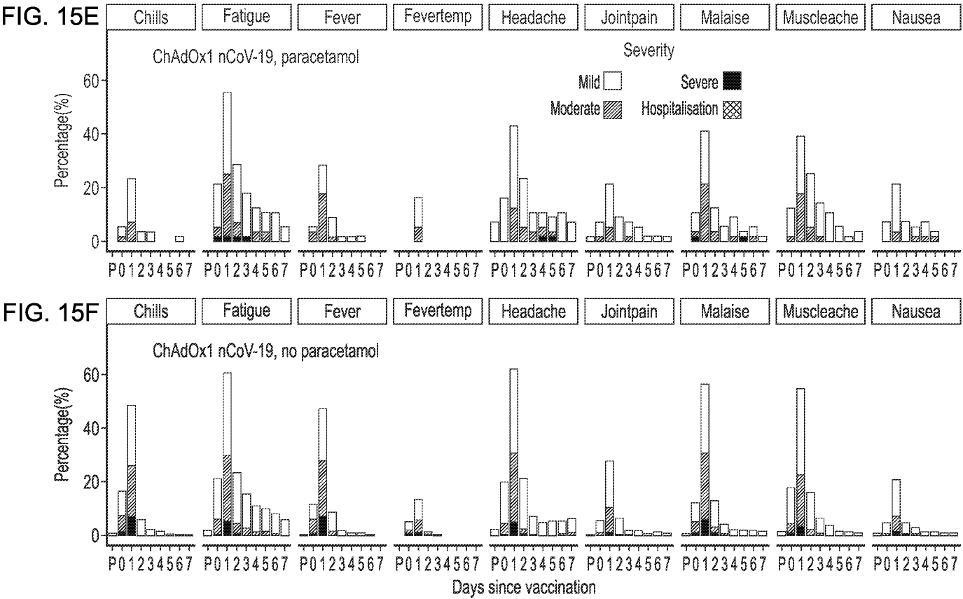

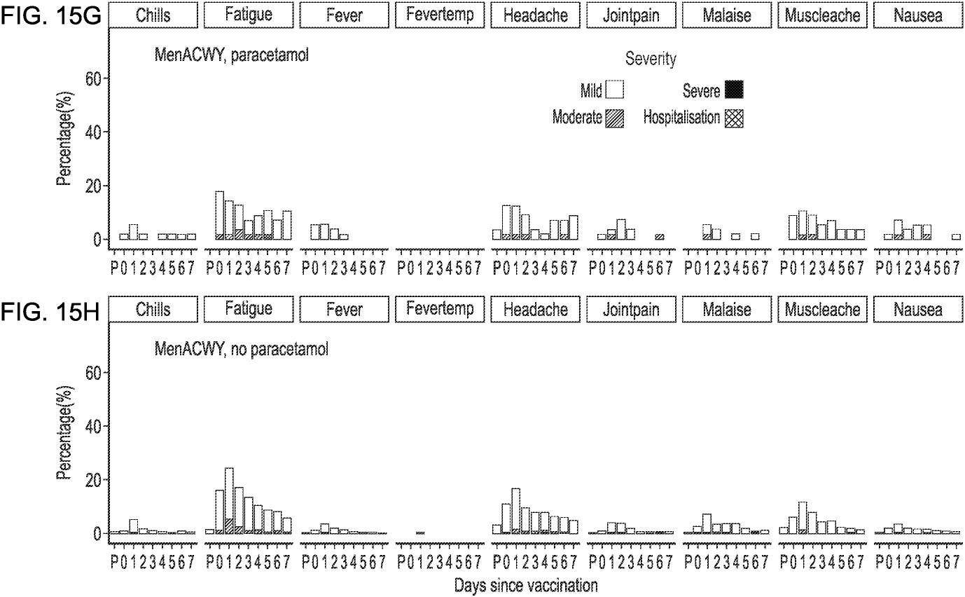

In those who did not receive prophylactic paracetamol, 67% of ChAdOx1 nCoV-19 participants and 38% of MenACWY participants reported pain after vaccination which was mostly mild to moderate in intensity. With prophylactic paracetamol pain was reduced to 50% in ChAdOx1 nCoV-19 participants and 32% of MenACWY participants. Tenderness of mostly mild intensity was reported by 83% ChAdOx1 nCoV-19 participants without paracetamol and 77% with paracetamol, and by 58% without paracetamol and 46% with paracetamol of MenACWY recipients. (FIG. 15, Table S2 below)

TABLE S2

Solicited adverse local and systemic reactions, with and without prophylactic paracetamol by e-diary

| Symptom | Vaccine Group | Paracetamol | None | Mild | Moderate | Severe | Hospitalisation |
|---|---|---|---|---|---|---|---|
| Pain | ChAdOx1 nCoV-19 | Paracetamol | 28 (50%, 36%-64%) | 22 (39%, 26%-53%) | 5 (9%, 3%-20%) | 1 (2%, 0%-10%) | 0 (0%, 0%-6%) |
| | | No Paracetamol | 159 (33%, 28%-37%) | 258 (53%, 48%-57%) | 66 (14%, 11%-17%) | 4 (1%, 0%-2%) | 0 (0%, 0%-1%) |
| | MenACWY | Paracetamol | 39 (68%, 55%-80%) | 15 (26%, 16%-40%) | 3 (5%, 1%-15%) | 0 (0%, 0%-6%) | 0 (0%, 0%-6%) |
| | | No Paracetamol | 296 (62%, 58%-67%) | 160 (34%, 29%-38%) | 18 (4%, 2%-6%) | 1 (0%, 0%-1%) | 0 (0%, 0%-1%) |
| Redness | ChAdOx1 nCoV-19 | Paracetamol | 55 (98%, 90%- | 0 (0%, 0%-6%) | 1 (2%, 0%-10%) | 0 (0%, 0%-6%) | 0 (0%, 0%-6%) |
| | | No Paracetamol | 100%) 472 (97%, 95%-98%) | 8 (2%, 1%-3%) | 6 (1%, 0%-3%) | 1 (0%, 0%-1%) | 0 (0%, 0%-1%) |
| | MenACWY | Paracetamol | 56 (98%, 91%-100%) | 0 (0%, 0%-6%) | 1 (2%, 0%-9%) | 0 (0%, 0%-6%) | 0 (0%, 0%-6%) |
| | | No Paracetamol | 465 (98%, 96%-99%) | 4 (1%, 0%-2%) | 6 (1%, 0%-3%) | 0 (0%, 0%-1%) | 0 (0%, 0%-1%) |
| Warmth | ChAdOx1 nCoV-19 | Paracetamol | 45 (80%, 68%-90%) | 10 (18%, 9%-30%) | 1 (2%, 0%-10%) | 0 (0%, 0%-6%) | 0 (0%, 0%-6%) |
| | | No Paracetamol | 365 (75%, 71%-79%) | 121 (25%, 21%-29%) | 1 (0%, 0%-1%) | 0 (0%, 0%-1%) | 0 (0%, 0%-1%) |
| | MenACWY | Paracetamol | 47 (82%, 70%-91%) | 10 (18%, 9%-30%) | 0 (0%, 0%-6%) | 0 (0%, 0%-6%) | 0 (0%, 0%-6%) |
| Itch | ChAdOx1 nCoV-19 | Paracetamol | 49 (88%, 76%-95%) | 6 (11%, 4%-22%) | 1 (2%, 0%-10%) | 0 (0%, 0%-6%) | 0 (0%, 0%-6%) |
| | | No Paracetamol | 452 (93%, 90%-95%) | 35 (7%, 5%-10%) | 0 (0%, 0%-1%) | 0 (0%, 0%-1%) | 0 (0%, 0%-1%) |
| | MenACWY | Paracetamol | 55 (96%, 88%-100%) | 2 (4%, 0%-12%) | 0 (0%, 0%-6%) | 0 (0%, 0%-6%) | 0 (0%, 0%-6%) |
| | | No Paracetamol | 450 (95%, 92%-97%) | 23 (5%, 3%-7%) | 2 (0%, 0%-2%) | 0 (0%, 0%-1%) | 0 (0%, 0%-1%) |
| Swelling | ChAdOx1 nCoV-19 | Paracetamol | 55 (98%, 90%-100%) | 0 (0%, 0%-6%) | 1 (2%, 0%-10%) | 0 (0%, 0%-6%) | 0 (0%, 0%-6%) |
| | | No Paracetamol | 466 (96%, 93%-97%) | 10 (2%, 1%-4%) | 10 (2%, 1%-4%) | 1 (0%, 0%-1%) | 0 (0%, 0%-1%) |
| | MenACWY | Paracetamol | 53 (93%, 83%-98%) | 3 (5%, 1%-15%) | 1 (2%, 0%-9%) | 0 (0%, 0%-6%) | 0 (0%, 0%-6%) |
| | | No Paracetamol | 461 (97%, 95%-98%) | 8 (2%, 1%-3%) | 6 (1%, 0%-3%) | 0 (0%, 0%-1%) | 0 (0%, 0%-1%) |
| Induration | ChAdOx1 nCoV-19 | Paracetamol | 56 (100%, 94%-100%) | 0 (0%, 0%-6%) | 0 (0%, 0%-6%) | 0 (0%, 0%-6%) | 0 (0%, 0%-6%) |
| | | No Paracetamol | 470 (97%, 94%-98%) | 13 (3%, 1%-5%) | 3 (1%, 0%-2%) | 1 (0%, 0%-1%) | 0 (0%, 0%-1%) |
| | MenACWY | Paracetamol | 54 (95%, 85%-99%) | 3 (5%, 1%-15%) | 0 (0%, 0%-6%) | 0 (0%, 0%-6%) | 0 (0%, 0%-6%) |
| Tenderness | ChAdOx1 nCoV-19 | Paracetamol | 13 (23%, 13%-36%) | 38 (68%, 54%-80%) | 5 (9%, 3%-20%) | 0 (0%, 0%-6%) | 0 (0%, 0%-6%) |
| | | No Paracetamol | 84 (17%, 14%-21%) | 333 (68%, 64%-72%) | 66 (14%, 11%-17%) | 4 (1%, 0%-2%) | 0 (0%, 0%-1%) |
| | MenACWY | Paracetamol | 31 (54%, 41%-68%) | 26 (46%, 32%-59%) | 0 (0%, 0%-6%) | 0 (0%, 0%-6%) | 0 (0%, 0%-6%) |
| | | No Paracetamol | 201 (42%, 38%-47%) | 256 (54%, 49%-58%) | 17 (4%, 2%-6%) | 1 (0%, 0%-1%) | 0 (0%, 0%-1%) |
| Feverishness | ChAdOx1 nCoV-19 | Paracetamol | 36 (64%, 50%-77%) | 8 (14%, 6%-26%) | 12 (21%, 12%-34%) | 0 (0%, 0%-6%) | 0 (0%, 0%-6%) |
| | | No Paracetamol | 237 (49%, 44%-53%) | 99 (20%, 17%-24%) | 111 (23%, 19%-27%) | 40 (8%, 6%-11%) | 0 (0%, 0%-1%) |
| | MenACWY | Paracetamol | 52 (91%, 81%-97%) | 5 (9%, 3%-19%) | 0 (0%, 0%-6%) | 0 (0%, 0%-6%) | 0 (0%, 0%-6%) |
| | | No Paracetamol | 437 (92%, 89%-94%) | 36 (8%, 5%-10%) | 2 (0%, 0%-2%) | 0 (0%, 0%-1%) | 0 (0%, 0%-1%) |
| Fever ≥ 38° C. | ChAdOx1 nCoV-19 | Paracetamol | 47 (84%, 72%-92%) | 6 (11%, 4%-22%) | 3 (5%, 1%-15%) | 0 (0%, 0%-6%) | 0 (0%, 0%-6%) |
| | | No Paracetamol | 400 (82%, 78%-85%) | 46 (9%, 7%-12%) | 33 (7%, 5%-9%) | 8 (2%, 1%-3%) | 0 (0%, 0%-1%) |
| | MenACWY | Paracetamol | 57 (100%, 94%-100%) | 0 (0%, 0%-6%) | 0 (0%, 0%-6%) | 0 (0%, 0%-6%) | 0 (0%, 0%-6%) |
| | | No Paracetamol | 473 (100%, 98%-100%) | 2 (0%, 0%-2%) | 0 (0%, 0%-1%) | 0 (0%, 0%-1%) | 0 (0%, 0%-1%) |
| | | No Paracetamol | 215 (44%, 40%-49%) | 121 (25%, 21%-29%) | 112 (23%, 19%-27%) | 39 (8%, 6%-11%) | 0 (0%, 0%-1%) |
| Chills | ChAdOx1 nCoV-19 | Paracetamol | 41 (73%, 60%-84%) | 10 (18%, 9%-302%) | 5 (9%, 3%-20%) | 0 (0%, 0%-6%) | 0 (0%, 0%-6%) |
| | | No Paracetamol | 215 (44%, 40%-49%) | 121 (25%, 21%-29%) | 112 (23%, 19%-27%) | 39 (8%, 6%-11%) | 0 (0%, 0%-1%) |
| | MenACWY | Paracetamol | 52 (91%, 81%-97%) | 5 (9%, 3%-19%) | 0 (0%, 0%-6%) | 0 (0%, 0%-6%) | 0 (0%, 0%-6%) |
| | | No Paracetamol | 429 (90%, 87%-93%) | 42 (9%, 6%-12%) | 4 (1%, 0%-2%) | 0 (0%, 0%-1%) | 0 (0%, 0%-1%) |
| Joint pain | ChAdOx1 nCoV-19 | Paracetamol | 40 (71%, 58%-83%) | 12 (21%, 12%-34%) | 4 (7%, 2%-17%) | 0 (0%, 0%-6%) | 0 (0%, 0%-6%) |
| | | No Paracetamol | 337 (69%, 65%-73%) | 97 (20%, 16%-24%) | 47 (10%, 7%-13%) | 6 (1%, 0%-3%) | 0 (0%, 0%-1%) |
| | MenACWY | Paracetamol | 51 (89%, 78%-96%) | 4 (7%, 2%-17%) | 2 (4%, 0%-12%) | 0 (0%, 0%-6%) | 0 (0%, 0%-6%) |
| | | No Paracetamol | 429 (90%, 87%- | 37 (8%, 6%-11%) | 8 (2%, 1%-3%) | 1 (0%, 0%-1%) | 0 (0%, 0%-1%) |
| Muscle ache | ChAdOx1 nCoV-19 | Paracetamol | 93%) 29 (52%, 38%-65%) | 14 (25%, 14%-38%) | 13 (23%, 13%-36%) | 0 (0%, 0%-6%) | 0 (0%, 0%-6%) |
| | | No Paracetamol | 193 (40%, 35%-44%) | 168 (34%, 30%-39%) | 107 (22%, 18%-26%) | 19 (4%, 2%-6%) | 0 (0%, 0%-1%) |
| | MenACWY | Paracetamol | 42 (74%, 60%-84%) | 14 (25%, 14%-38%) | 1 (2%, 0%-9%) | 0 (0%, 0%-6%) | 0 (0%, 0%-6%) |
| | | No Paracetamol | 358 (75%, 71%-79%) | 104 (22%, 18%-26%) | 13 (3%, 1%-5%) | 0 (0%, 0%-1%) | 0 (0%, 0%-1%) |
| Fatigue | ChAdOx1 nCoV-19 | Paracetamol | 16 (29%, 17%-42%) | 23 (41%, 28%-55%) | 14 (25%, 14%-38%) | 3 (5%, 1%-15%) | 0 (0%, 0%-6%) |
| | MenACWY | Paracetamol | 31 (54%, 41%-68%) | 19 (33%, 21%-47%) | 7 (12%, 5%-24%) | 0 (0%, 0%-6%) | 0 (0%, 0%-6%) |
| | | No Paracetamol | 249 (52%, 48%-57%) | 169 (36%, 31%-40%) | 56 (12%, 9%-15%) ) | 1 (0%, 0%-1%) | 0 (0%, 0%-1%) |
| Headache | ChAdOx1 nCoV-19 | Paracetamol | 22 (39%, 26%-53%) | 25 (45%, 31%-59%) | 8 (14%, 6%-26%) | 1 (2%, 0%-10%) | 0 (0%, 0%-6%) |
| | | No Paracetamol | 156 (32%, 28%-36%) | 168 (34%, 30%-39%) | 136 (28%, 24%-32%) | 27 (6%, 4%-8%) | 0 (0%, 0%-1%) |
| | MenACWY | Paracetamol | 36 (63%, 49%-76%) | 18 (32%, 20%-45%) | 3 (5%, 1%-15%) | 0 (0%, 0%-6%) | 0 (0%, 0%-6%) |
| | | No Paracetamol | 280 (59%, 54%-63%) | 166 (35%, 31%-39%) | 26 (5%, 4%-8%) | 3 (1%, 0%-2%) | 0 (0%, 0%-1%) |
| Malaise | ChAdOx1 nCoV-19 | Paracetamol | 29 (52%, 38%-65%) | 11 (20%, 10%-32%) | 14 (25%, 14%-38%) | 2 (4%, 0%-12%) | 0 (0%, 0%-6%) |
| | | No Paracetamol | 191 (39%, 35%-44%) | 136 (28%, 24%-32%) | 124 (25%, 22%-30%) | 36 (7%, 5%-10%) | 0 (0%, 0%-1%) |
| | MenACWY | Paracetamol | 51 (89%, 78%-96%) | 5 (9%, 3%-19%) | 1 (2%, 0%-9%) | 0 (0%, 0%-6%) | 0 (0%, 0%-6%) |
| | | No Paracetamol | 393 (83%, 79%-86%) | 70 (15%, 12%-18%) | 11 (2%, 1%-4%) | 1 (0%, 0%-1%) | 0 (0%, 0%-1%) |
| Nausea | ChAdOx1 nCoV-19 | Paracetamol | 37 (66%, 52%-78%) | 14 (25%, 14%-38%) | 5 (9%, 3%-20%) | 0 (0%, 0%-6%) | 0 (0%, 0%-6%) |
| | | No Paracetamol | 363 (75%, 70%-78%) | 81 (17%, 13%-20%) | 33 (7%, 5%-9%) | 10 (2%, 1%-4%) | 0 (0%, 0%-1%) |
| | MenACWY | Paracetamol | 46 (81%, 68%-90%) | 10 (18%, 9%-30%) | 1 (2%, 0%-9%) | 0 (0%, 0%-6%) | 0 (0%, 0%-6%) |
| No Paracetamol | 427 (90%, 87%- 92%) | | 41 (9%, 6%-12%) | | 7 (1%, 1%-3%) | 0 (0%, 0%-1%) | 0 (0%, 0%-1%) |

TABLE S3

Solicited Local and System Reactions after Prime and Boost vaccinations in Group 3 participants

| Symptom | 1^{st} dose (N=10) | | | | | 2^{nd} dose (N=10) | | | | |
|---|---|---|---|---|---|---|---|---|---|---|
| | None | Mile | Moderate | Servere | Hospitalisation | None | Mild | Moderate | Severe | Hosonpitalisati om |
| Pain | 5 (50%, 19%-81%) | 4 (40%, 12%-74%) | 1 (10%, 0%- 45% | 0 (0%, 0%- 31%) | 0 (0%, 0%- 31%) | 8 (80%, 44%-97%) | 2 (20%, 3%-56%) | 0 (0%, 0%-31%) | 0 (0%, 0%- 31%) | 0 (0%, 0%-31%) |
| Redness | 10 (100%, 69%-100%) | 0 (0%, 0%-31%) | 0 (0%, 0%– 31%) | 0 (0%, 0%-31%) | 0 (0%, 0%-31%) | 10 (100%, 69%-100% | 0 (0%, 0%-31%) | 0 (0%, 0%-31%) | 0 (0%, 0%-31%) | 0 (0%, 0%-31%) |
| Warmth | 8 (80%, 44%-97% | 20 (20%, 3%-56%) | 0 (0%, 0%-31%) | 0 (0%, 0%-31%) | 0 (0%, 0%-31%) | 8 (80%, 44%-97%) | 2 (20%, 3%-56%) | 0 (0%, 0%-31%) | 0 (0%, 0%-31%) | 0 (0%, 0%-31%) |
| Itch | 9 (90%, 55%- 100% | 1(10%, 0%-45%) | 0 (0%, 0%-31%) | 0 (0%, 0%-31%) | 0 (0%, 0%- 31%) | 9 (90%, 55%-100% | 1(10%, 0%-45%) | 0 (0%, 0%-31%) | 0 (0%, 0%-31%) | 0 (0%, 0%-31%) |
| Swelling | 10 (100%, 69%-100%) | 0 (0%, 0%-31%) | 0 (0%, 0%-31%) | 0 (0%, 0%-31%) | 0 (0%, 0%-31%) | 10 (100%, 69%-100% | 0 (0%, 0%-31%) | 0 (0%, 0%-31%) | 0 (0%, 0%-31%) | 0 (0%, 0%-31%) |
| Induration | 10 (100%, 69%-100%) | 0 (0%, 0%-31%) | 0 (0%, 0%-31%) | 0 (0%, 0%-31%) | 0 (0%, 0%-31%) | 10 (100%, 69%-100%) | 0 (0%, 0%-31%) | 0 (0%, 0%-31%) | 0 (0%, 0%-31%) | 00 (0%, 0%-31%) |
| Tenderness | 5 (50%, 19%-81% | 4 (40%, 12%-74% | 1 (10%, 0%-45%) | 0 (0%, 0%-31%) | 0 (0%, 0%-31%) | 5 (50%, 19%-81% | 5 (50%, 19%-81% | 0 (0%, 0%-31%) | 0 (0%, 0%-31%) | 0 (0%, 0%-31%) |
| Feverishness | 3 (30%, 7%-65%) | 2 (20%, 3%-56% | 5 (50%, 19%-81% | 0 (0%, 0%-31%) | 0 (0%, 0%-31%) | 8 (80%, 44%-97%) | 1 (10%, 0%-45%) | 1 (10%, 0%-45%) | 0 (0%, 0%-31%) | 0 (0%, 0%-31%) |
| Fever >= 38 | 5 (50%, 19%-81% | 5 (50%, 19%-81% | 0 (0%, 0%-31%) | 0 (0%, 0%-31%) | 0 (0%, 0%-31%) | 10 (100%, 69%-100% | 0 (0%, 0%-31%) | 0 (0%, 0%-31%) | 0 (0%, 0%-31%) | 31%) 0 (0%, 0%- |
| Chills | 2 (20%, 3%-56% | 5 (50%, 19%-81% | 3 (30%, 7%-65%) | 0 (0%, 0%-31%) | 0 (0%, 0%-31%) | 8 (80%, 44%-97%) | 2 (20%, 3%-56% | 0 (0%, 0%-31%) | 0 (0%, 0%-31%) | 31%) 0 (0%, 0%- |
| Joint pain | 7 (70%, 35%-93% | 2 (20%, 3%-56% | 1 (10%, 0%- 45% | 0 (0%, 0%-31%) | 0 (0%, 0%-31%) | 10 (100%, 69%-100% | 0 (0%, 0%-31%) | 0 (0%, 0%-31%) | 0 (0%, 0%-31%) | 31%) 0 (0%, 0%- |
| Muscle ache | 4 (40%, 12%-74% | 4 (40%, 12%-74% | 2 (20%, 3%-56% | 0 (0%, 0%-31%) | 0 (0%, 0%-31%) | 8 (80%, 44%-97%) | 2 (20%, 3%-56% | 0 (0%, 0%-31%) | 0 (0%, 0%-31%) | 31%) 0 (0%, 0%- |
| Fatigue | 5 (50%, 19%-81% | 4 (40%, 12%-74% | 1 (10%, 0%-45%) | 0 (0%, 0%-31%) | 0 (0%, 0%-31%) | 6 (60%, 26%-88% | 3 (30%, 7%-65%) | 1 (10%, 0%- 45% | 0 (0%, 0%-31%) | 31%) 0 (0%, 0%- |
| Headache | 3 (30%, 7%-65%) | 3 (30%, 7%-65%) | 4 (40%, 12%-74% | 0 (0%, 0%-31%) | 0 (0%, 0%-31%) | 6 (60%, 26%-88% | 4 (40%, 12%-74% | 0 (0%, 0%-31%) | 0 (0%, 0%-31%) | 31%) 0 (0%, 0%- |
| Malaise | 6 (60%, 26%-88% | 0 (0%, 0%-31%) | 4 (40%, 12%-74% | 0 (0%, 0%-31%) | 31%) | 8 (80%, 44%-97%) | 2 (20%, 3%-56% | 0 (0%, 0%-31%) | 0 (0%, 0%-31%) | 31%) |
| Nausea | 6 (60%, 26%-88% | 1 (10%, 0%- 45% | 3 (30%, 7%-65%) | 0 (0%, 0%-31%) | 0 (0%, 0%-31%) | 8 (80%, 44%-97% | 2 (20%, 3%-56% | 6 (60%, 26%-88% | 6 (60%, 26%-88% | 0 (0%, 0%-31%) |

Fatigue and headache were the most commonly reported systemic reactions. Fatigue was reported in 70% and 71% (no paracetamol, paracetamol) ChAdOx1 nCoV-19 participants and 48% and 46% (no paracetamol, paracetamol) MenACWY recipients, whilst headaches occurred in 68% and 61% (no paracetamol, paracetamol) ChAdOx1 nCoV-19 participants and 41% 37% (no paracetamol, paracetamol) MenACWY recipients.

Other systemic adverse reactions were common in the ChAdOx1 nCoV-19 group: muscle ache 60%, 48% (no paracetamol, paracetamol) malaise 61% 48% (no paracetamol, paracetamol); chills 56%, 37% (no paracetamol, paracetamol); and feeling feverish 51%, 36% (no paracetamol, paracetamol). 18% and 16% of ChAdOx1 nCoV-19 participants (no paracetamol, paracetamol) reported a temperature ≥ 38° C., and 2% had a temperature ≥ 39° C. without paracetamol. In comparison < 0.5% of those receiving MenACWY reported a fever ≥ 38° C., and none of those who received prophylactic paracetamol. (FIG. 15, Table S2).

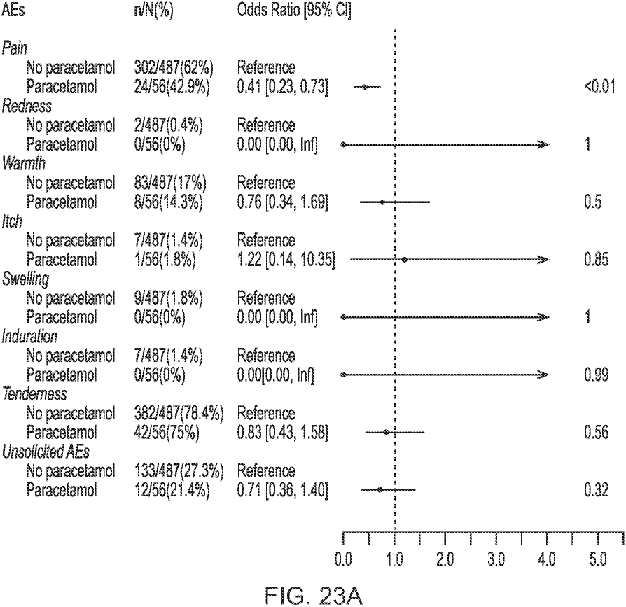

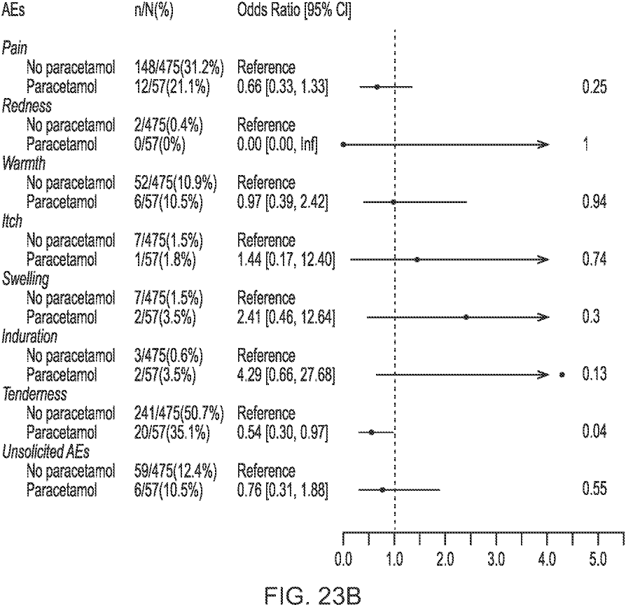

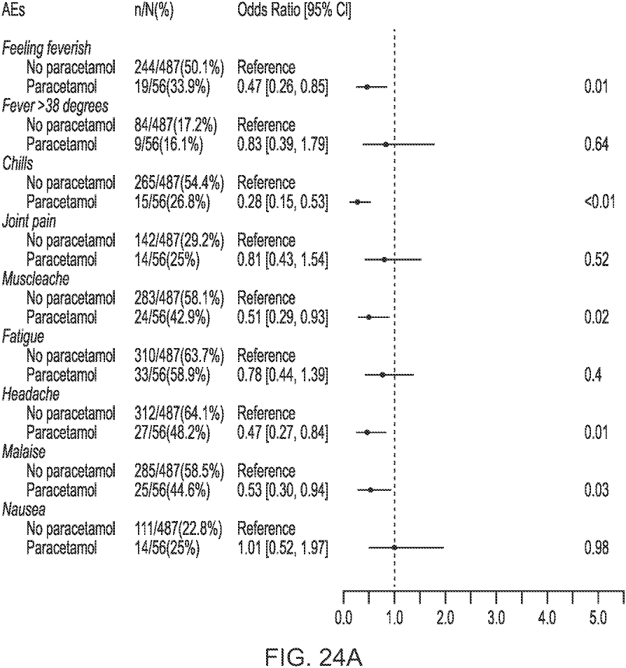

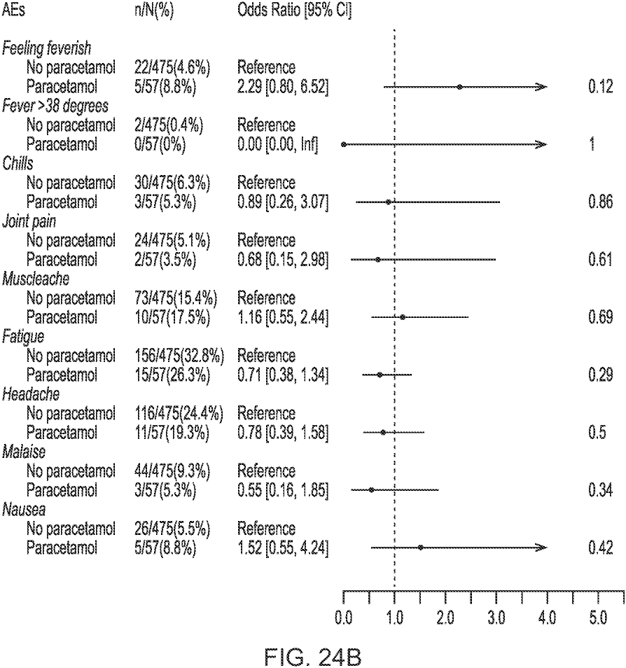

The severity and intensity of local and systemic reactions was highest on day 1 of the study (FIG. 15). Adjusted analysis of the effect of prophylactic paracetamol on adverse reactions of any severity in the first 2 days after vaccination after vaccination with ChAdOx1 nCoV-19 showed significant reductions in pain, feeling feverish, chills, muscle ache, headache, and malaise (FIGS. 23 and 24).

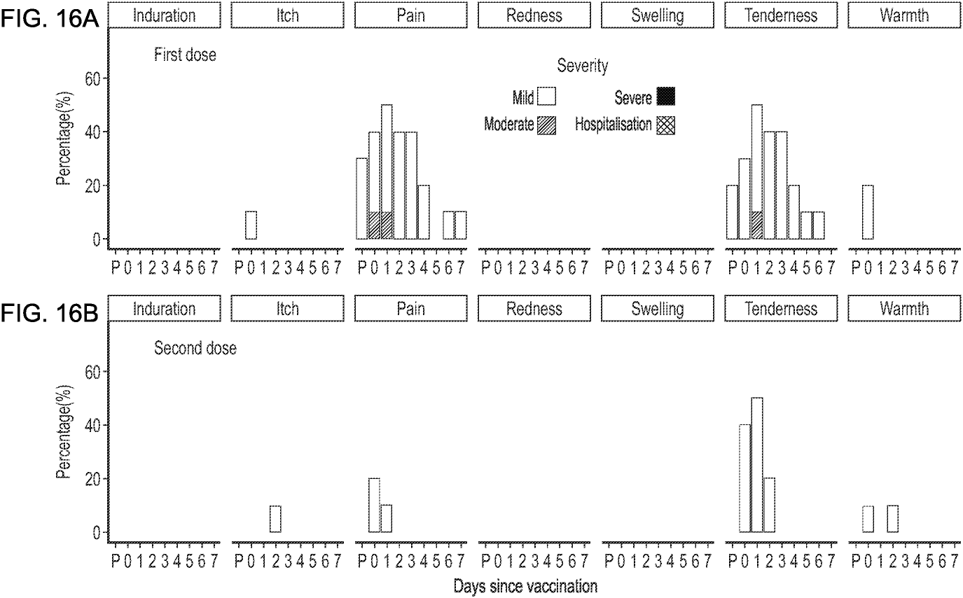

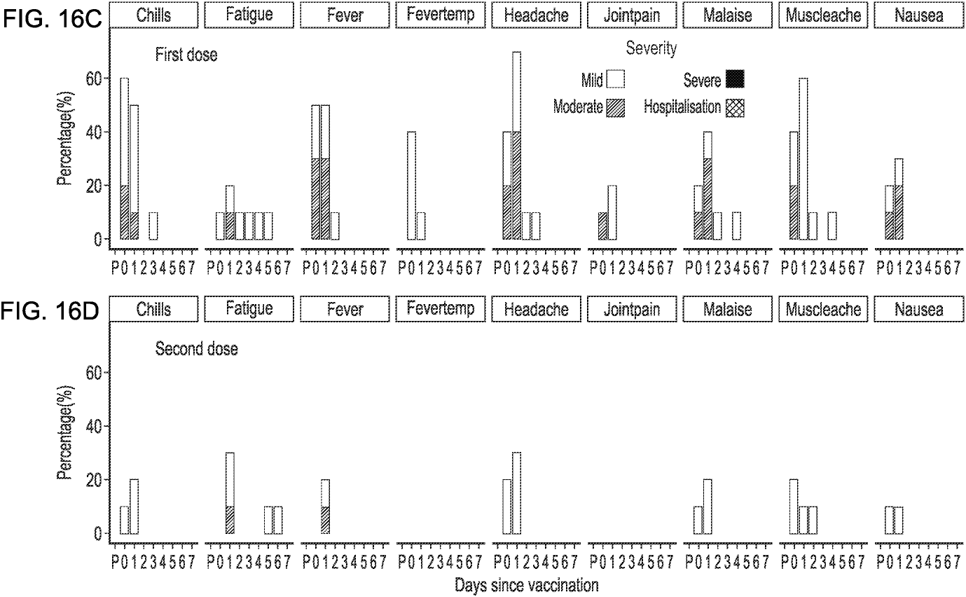

Ten people received a booster dose of ChAdOx1 nCoV-19 and solicited local and systemic reactions were measured for 7 days after both the prime and booster doses. The reactogenicity profile after the second dose appeared less severe in this subset, although the number of participants was small in this group, leading to wide confidence intervals (FIG. 16, Table S3). Unsolicited adverse events in the 28 days following vaccination considered to be possibly, probably, or definitely related to ChAdOx1 nCoV-19 were predominantly mild and moderate in nature and resolved within the follow-up period. Laboratory adverse events considered to be at least possibly related to the study intervention were self-limiting and predominantly mild or moderate in severity (see table). Transient haematological changes from baseline (leukopenia, lymphopenia, neutropenia and thrombocytopenia) were observed in a % of participants in the ChAdOx1 nCoV-19 arm, compared with a % receiving MenACWY.

There was one serious adverse event consisting of a new diagnosis of haemolytic anaemia, occurring 9 days after vaccination. The participant was clinically well throughout. The event was reported as a SUSAR relating to the MenACWY vaccine.

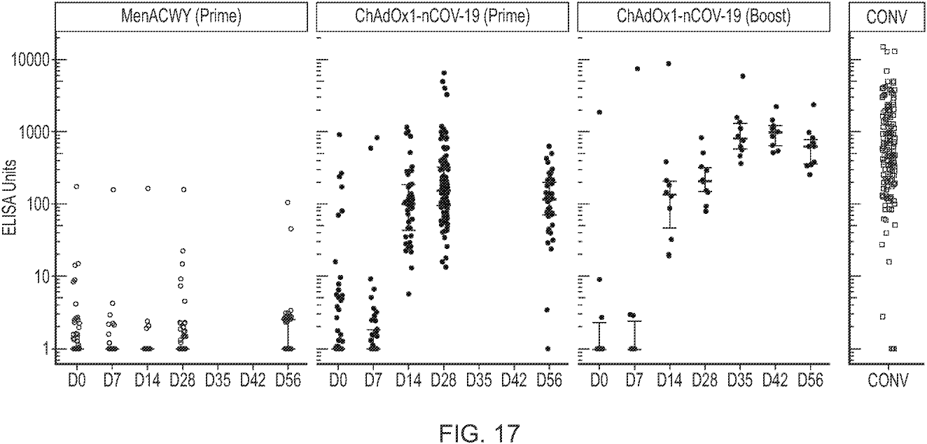

In the ChAdOx1 nCoV-19 group, antibodies against SARS-CoV-2 spike protein peaked by day 28 (median 157, IQR 96, 317) and remained elevated to day 56 (median 119, IQR 70, 203) in 43 participants who received only one dose, and increased to a median of 639 (IQR 360, 792) in 10 participants who received a booster dose (FIG. 17).

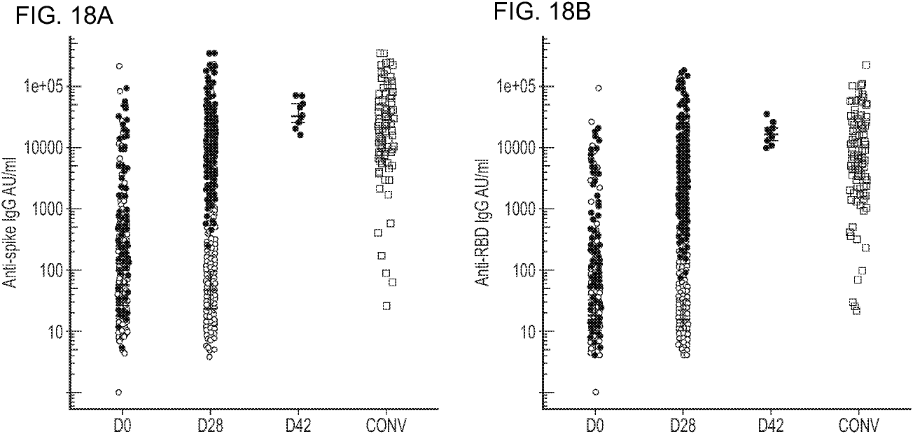

Similar increases in antibody responses to both the spike protein and the receptor binding domain by day 28 and after a booster dose were observed when measured on a multiplex assay (FIG. 18).

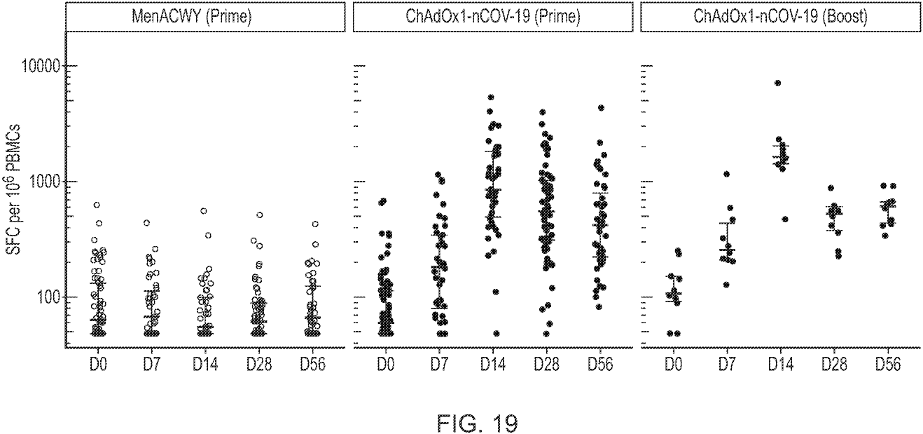

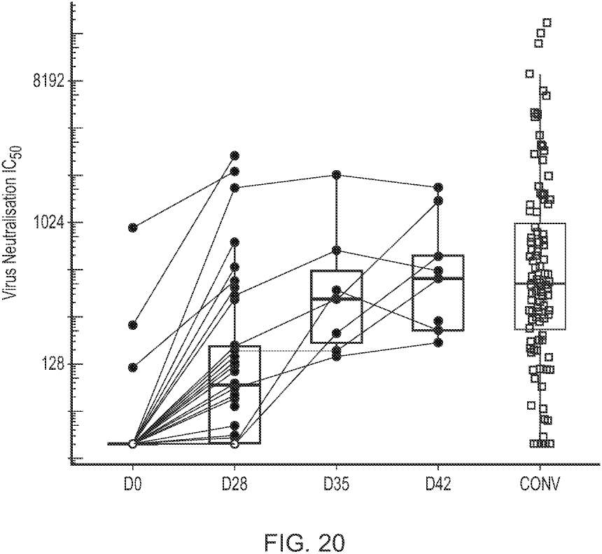

IFN-gamma ELISpot responses against SARS-CoV-2 spike protein peaked at 856 spot-forming cells per million PBMC (SFC) at day 14 (IQR 493.3-1802 SFC], declining to 424 (IQR 221, 799) by day 56 post-vaccination (FIG. 19).

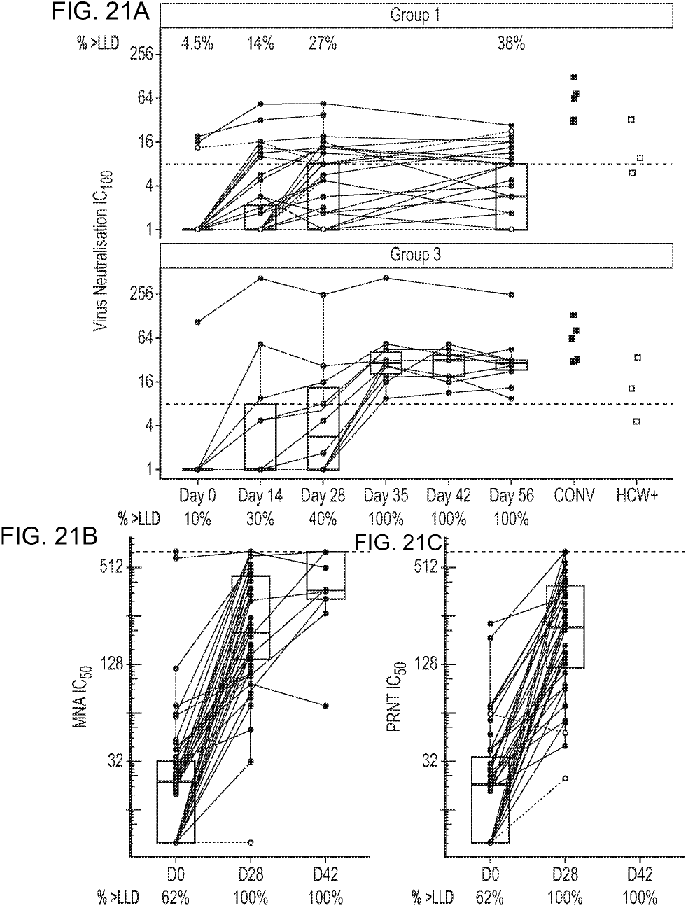

After a single dose of ChAdOx1 nCoV-19, 27% of participants had antibody levels sufficient to induce complete inhibition of viral cell entry by day 28, rising to 38% of participants by day 56, in a SARS-CoV-2 virus neutralization assay measuring IC100 (Marburg). After a booster dose, viral titres induced complete inhibition of SARS-CoV-2 virus in all participants (FIG. 21 top panels).

In two separate live virus neutralization assay performed at Public Health England, 100% of participants achieved PRNT50 titres at day 28, and similar results were obtained by micro-neutralization (FIG. 21 lower panels). The pseudo-virus neutralization assay titres correlated with live virus neutralization assay titres and ELISA.

### Discussion of Example 15

Our findings show that the candidate ChAdOx1 nCoV-19 vaccine given as a single dose was safe and tolerated, despite a greater reactogenicity profile than the control vaccine, MenACWY. No serious adverse reactions to ChAdOx1 nCoV-19 occurred. The majority of AEs reported were mild or moderate in severity, and all were self-limiting. The profile of adverse events reported here is similar to that for other ChAdOx1 vectored vaccines and other closely related simian adenoviruses, such as ChAdOx2, ChAd3, and ChAd63 vectored vaccines expressing multiple different antigens (ChAdOx1, Folegatti 2020 ChAdOx2, Vaccines 2019, 7, 40; doi:10.3390/vaccines7020040 ChAd63, doi: 10.1038/s41598-018-21630-4 ChAd3, doi: 10.1056/NEJMoa1411627) at this dose level. A dose of 5×10¹⁰ vp was chosen based on our previous experience with ChAdOx1 MERS, where despite increased reactogenicity, a dose response relationship with neutralising antibodies was observed.⁷ The protocol was written when the pandemic was accelerating in the UK and a single higher dose was chosen to provide the highest chance of rapid induction of neutralising antibody. In the context of a pandemic wave where a single higher, but more reactogenic, dose may be more likely to rapidly induce protective immunity, the use of prophylactic paracetamol appears to increase tolerability and would reduce confusion with COVID19 symptoms that might be caused by short-lived vaccine-related symptoms.

We demonstrate that a single dose of ChAdOx1 nCoV-19 elicits spike-specific antibodies by day 14 in 64% of vaccinees, which were evident in 95% of vaccinees by day 28. A small number (3%) of participants had detectable antibodies against SARS-CoV-2 spike protein before vaccination. These pre-existing responses are likely due to asymptomatic infection as potential participants with recent COVID-19-like symptoms or a positive PCR test for SARS-CoV-2 were excluded from the study. Of the three individuals seropositive on the day of vaccination in our in -house ELISA, two received ChAdOx1 nCoV-19 and mounted a booster response.

Neutralizing antibodies targeting different epitopes of the spike glycoprotein and have been associated with protection from COVID disease in early preclinical rhesus macque studies (Barouch). Whilst a correlate of protection has not been defined for COVID-19, high levels of neutralising antibodies have been demonstrated in convalescent individuals, with a wide range, as confirmed in our study. Neutralising antibodies against live SARS-CoV-2 virus were detected in 27% and 100% of participants by day 28 (IC100 and IC50 respectively), using different assays. Neutralising antibody titres and seroconversion rates were increased by a two-dose regimen, and further investigation of this approach is underway. The correlation of neutralisation assays with IgG quantitation, indicates that, if confirmed, simple ELISA may be sufficient to predict protection, should neutralising antibody be shown to be protective in humans. We have presented data from 3 different live neutralising antibody assays and a pseudo-neutralisation assay, which show tight correlation with each other but give very different neutralising antibody titres. This issue highlights the urgent need for centralised laboratory infrastructure to allow bridging between vaccine candidates and accelerate the availability of multiple products to provide the global capacity to end the pandemic. If any one candidate demonstrates efficacy, bridging this result to other candidate vaccines through rigorously conducted laboratory assays will become a critical issue for global health.

Importantly, in COVID patients, there is accumulating data to suggest T-cell responses play an important role in COVID-19 disease mitigation; individuals who were exposed but asymptomatic developed a robust memory T cell response which prevented disease in the absence of a measurable humoral response.¹⁰⁻¹² ChAdOx1 nCoV-19 vaccination resulted in significant increases in SARS CoV-2 spike-specific T-cell responses as early as day 7, peaking at day 14 and maintained out to day 28 and was ? by booster vaccination.

Severe and fatal cases of SARS CoV-2 disproportionally affect older individuals. Therefore, it is paramount that vaccines developed against SARS CoV-2 are suitable for administration in older age groups. Immunogenicity of a ChAdOx1 vectored vaccine against influenza has been demonstrated in older adults (50-78 years of age). In the next stage of clinical development, older adults will be recruited and assessed for safety and immunogenicity of ChAdOx1 nCoV-19 to be given as a single or two-dose administration regimen. In addition, phase III trials, which are underway in the UK, Brazil and South Africa, will evaluate vaccine efficacy.

Limitations of this study include the short follow-up and single-blinded design. The participants recruited in this study will be followed-up for at least 1 year and further safety, tolerability and immunogenicity (in addition to efficacy) results will be reported when data are available (Weingartl, H. et al. Immunization with modified vaccinia virus Ankara-based recombinant vaccine against severe acute respiratory syndrome is associated with enhanced hepatitis in ferrets. J Virol 78, 12672-12676, doi:10.1128/JVI.78.22.12672-12676.2004 (2004). Bolles, M. et al. A double-inactivated severe acute respiratory syndrome coronavirus vaccine provides incomplete protection in mice and induces increased eosinophilic proinflammatory pulmonary response upon challenge. J Virol 85, 12201-12215, doi:10.1128/JVI.06048-11 (2011). Liu, L. et al. Anti-spike IgG causes severe acute lung injury by skewing macrophage responses during acute SARS-CoV infection. JCI Insight 4, doi:10.1172/jci.insight.123158 (2019).).

In conclusion, ChAdOx1 nCoV-19 was safe, tolerated and immunogenic, reactogenicity was reduced with paracetamol. A single dose elicited both humoral and cellular responses against SARS-CoV-2, with a booster immunisation augmenting neutralising antibody titres. The preliminary results of this first-in-human clinical trial support clinical development progression into phase 2 and 3 trials. Older age groups with comorbidities, health care workers and those with higher risk for SARS-CoV-2 exposure will be recruited and assessed for efficacy, safety and immunogenicity of ChAdOx1 nCoV-19 to be given as a single or two-dose administration regimen in further trials conducted in the UK and overseas.

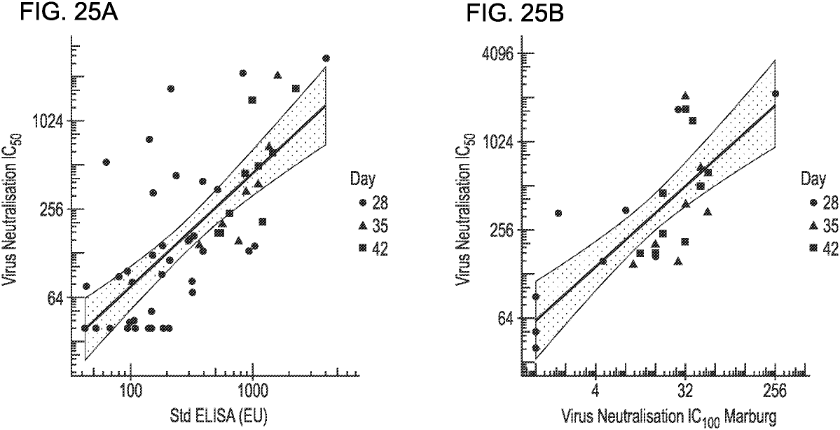

We also refer to FIG. 25 which shows pseudotype neutralisation (IC50) in ChAdOx1 nCoV-19 recipients correlates with standardised ELISA and with live virus neutralisation as measured by IC100 (Marburg).

We also refer to the table below - Summary statistics for antibody assays and t cell responses.

| Assay | Study Day | | MenACWY | | ChAdOx1 Prime | | ChAdOx1 Prime-Boost |
|---|---|---|---|---|---|---|---|
| | | N | Median [IQR] | N | Median [IQR] | N | Median [IQR] |
| Multiplex MSD -RBD IgG (AU/mL) | 0 | 487 | 15.6 [10.1, 32.7] | 488 | 18.5 [10.9, 38.8] | | |
| | 28 | 485 | 15.8 [10.2, 32.4] | 482 | 3182.5 [1426.3, 6800.4] | | |
| | 42 | | | | | 9 | 16825.4 [13118.9, 20937.9] |
| Multiplex MSD - Anti spike IgG (AU/mL) | 0 | 487 | 45.5 [26.1, 98.2] | 488 | 57.6 [29.1, 125.8] | | |
| | 28 | 485 | 43.9 [23.9, 94.5] | 482 | 10471.8 [5378.6, 20517.3] | | |
| | 42 | | | | | 9 | 33830.8 [25674.3, 52251.8] |
| Anti-spike IgG using Standardised ELISA (EU/mL) | 0 | 131 | 1 [1, 1] | 129 | 1 [1, 1] | 10 | 1 [1, 2.3] |
| | 7 | 44 | 1 [1, 1] | 44 | 1 [1, 1.8] | 10 | 1 [1, 2.4] |
| | 14 | 44 | 1 [1, 1] | | 102.7. [43.47, 186] | 10 | 137 [46.4, 206.8] |
| | 28 | 130 | 1 [1, 1] | 127 | 157.1 [96.2, 316.9] | 10 | 210.7 [149.4, 321.6] |
| | 35 | | | | | 10 | 821.1 [578.1, 1298.4] |
| | 42 | | | | | 9 | 997.5 [648.5, 1214] |
| | 56 | 44 | 1[1. 2.6] | 43 | 119 [70.3, 203.4] | 10 | 639.2 [648.5, 1214] |
| IFN-γ ELISpot response against SARS-CoV-2 spike protein (spot-forming | 0 | 73 | 64 [48, 132] | 71 | 60 [48, 114] | 10 | 108 [90.8, 150.2] |
| | 7 | 43 | 67.3 [48, 113] | 40 | 83.2 [79.5, 345] | 10 | 258 [209.8, 432.2] |
| | 14 | 44 | 55.3 [48, 99.3] | 43 | 856 [493.3, 1802] | 10 | 1642.3 [1423.7, 2009.5] |
| | 28 | 69 | 61.3 [48, 88] | 68 | 554.3 [311.3, 1017.7] | 10 | 528.7 [376.3, 603] |
| cells per million PBMC) | 56 | 42 | 66.7 [48, 123.3] | 43 | 424 [221.3, 798.7] | 10 | 614 [437.3, 666] |

### Summary

We conducted a phase I/II single blind randomised controlled trial to evaluate the safety, immunogenicity and efficacy of a chimpanzee adenovirus viral vector (ChAdOx1) vaccine expressing the SARS-CoV-2 spike protein, at a dose of 5x10¹⁰ viral particles, in healthy adults aged 18-55 years compared with a meningococcal conjugate vaccine (MenACWY) as control. A non-randomised subset of volunteers received a two-dose schedule. We assessed safety, and cellular and humoral immune responses. The study was registered at ISRCTN number 15281137 and ClinicalTrials.gov, NCT04324606.

### Findings

Between April 23^{rd} 2020 and May 21^{st} 1077 volunteers were randomised 1:1 to receive ChAdOx1 nCoV-19 or MenACWY vaccines. Local reactions were more common in the ChAdOx1nCoV-19 group including pain (67%) and tenderness (83%) at the injection site. Systemic reactions including headache (68%), fatigue (70%), chills (56%), feverishness (51%), malaise (61%) and muscle-ache (60%) were commonly reported, but reduced by use of prophylactic paracetamol, and lower after a second dose. There were no serious adverse events related to ChAdOx1nCoV-19. Spike protein T cell responses peaked at day 14. Anti-spike protein IgG responses were detectable by day 14 after immunisation, and continued to rise to day 28. IC50 live coronavirus neutralising antibody responses were detected in 100% of vaccinees one month after one dose (38% in IC100 assay) and in 100% after 2 doses(100% in IC100 assay), and were strongly correlated with ELISA antibody responses and responses in a pseudovirus neutralisation assay.

### Interpretation

ChAdOx1nCoV-19 was tolerable after vaccination with reactogenicity mitigated by use of prophylactic paracetamol. Spike protein IgG correlated with neutralising antibody responses and immunogenicity improved after a second dose.

Added value of this study: This study is the first clinical study of ChAdOx1nCoV-19 (AZD1222). The vaccine was safe and tolerated, with reduced reactogenicity when paracetamol was used prophylactically for the first 24 hours after vaccination. In the small group who received a second dose, reactogenicity was reduced after the second dose. Four-fold increases in humoral responses to SARS-CoV-2 spike protein were induced in 95% of participants by day 28 and cellular responses were induced in all participants by day 14. Neutralising antibodies were induced in a larger proportion of participants after a second vaccine dose. The study was done in the UK.

Implications of all the available evidence: A vaccine against SARS-CoV-2 could be used to prevent infection, disease and/or death in the whole population, with high risk populations such as hospital workers and older adults prioritised to receive vaccination. The immune correlates of protection against SARS-CoV-2 have not yet been determined. Immunisation with ChAdOx1-nCoV-19 results in rapid induction of humoral and cellular immune responses against SARS-CoV-2, with increased responses after a second dose. Further clinical studies, including in older adults, should be done with this vaccine.

### References to Example 15

1. World Health Organisation. Draft Landscape of COVID-19 candidate vaccines. 2020. https://www.who.int/publications/m/item/draft-landscape-of-covid-19-candidate-vaccines.

2. Fidler S, Stohr W, Pace M, et al. Antiretroviral therapy alone versus antiretroviral therapy with a kick and kill approach, on measures of the HIV reservoir in participants with recent HIV infection (the RIVER trial): a phase 2, randomised trial. Lancet 2020; 395(10227): 888-98.

3. Hanke T. Aiming for protective T-cell responses: a focus on the first generation conserved-region HIVconsv vaccines in preventive and therapeutic clinical trials. Expert Rev Vaccines 2019; 18(10): 1029-41.

4. Mothe B, Manzardo C, Sanchez-Bernabeu A, et al. Therapeutic Vaccination Refocuses T-cell Responses Towards Conserved Regions of HIV-1 in Early Treated Individuals (BCN 01 study). EClinicalMedicine 2019; 11: 65-80.

5. Coughlan L, Sridhar S, Payne R, et al. Heterologous Two-Dose Vaccination with Simian Adenovirus and Poxvirus Vectors Elicits Long-Lasting Cellular Immunity to Influenza Virus A in Healthy Adults. EBioMedicine 2018; 29: 146-54.

6. van Doremalen N, Haddock E, Feldmann F, et al. A single dose of ChAdOx1MERS provides protective immunity in rhesus macaques. Science Advances 2020; 6(24): eaba8399.

7. Folegatti PM, Bittaye M, Flaxman A, et al. Safety and immunogenicity of a candidate Middle East respiratory syndrome coronavirus viral-vectored vaccine: a dose-escalation, open-label, non-randomised, uncontrolled, phase 1 trial. Lancet Infect Dis 2020; 20(7): 816-26.

8. van Doremalen N, et al. ChAdOx1nCoV-19 vaccination prevents SARS-CoV-2 pneumonia in rhesus macaques BioRxiv (under review) 2020.

9. Dicks MD, Spencer AJ, Edwards NJ, et al. A novel chimpanzee adenovirus vector with low human seroprevalence: improved systems for vector derivation and comparative immunogenicity. PLoS One 2012; 7(7): e40385.

10. Grifoni A, Weiskopf D, Ramirez SI, et al. Targets of T Cell Responses to SARS-CoV-2 Coronavirus in Humans with COVID-19 Disease and Unexposed Individuals. Cell 2020; 181(7): 1489-501.e15.

11. Sekine T, Perez-Potti A, Rivera-Ballesteros O, et al. Robust T cell immunity in convalescent individuals with asymptomatic or mild COVID-19. bioRxiv 2020: 2020.06.29.174888.

12. Weiskopf D, Schmitz KS, Raadsen MP, et al. Phenotype and kinetics of SARS-CoV-2-specific T cells in COVID-19 patients with acute respiratory distress syndrome. Science Immunology 2020; 5(48): eabd2071.

### Example 16: Safety and Immunogenicity of ChAdOxl nCoV-19 Vaccine Administered in A Prime-Boost Regimen in Older Adult Humans (COV002)

Example 16 relates to a Phase ⅔ single blind, randomised controlled trial, it should be read in conjunction with the earlier examples, in particular Examples 11 and 15.

### Background

Older adults are at higher risk of severe disease and have a higher mortality if they develop COVID-19, and are therefore a priority for immunisation should an efficacious vaccine be developed. Immunogenicity of vaccines is often poorer in older adults as a result of immunosenescence. We have recently reported the immunogenicity of a novel viral vectored vaccine, ChAdOx1nCoV-19, in young adults, and now describe the safety and immunogenicity of this vaccine in older adults.

Two recombinant viral vectored vaccines have been tested in clinical trials. A single dose adenovirus-5 (Ad5) vector-based vaccine (CanSino Biological/Beijing Institute of Biotechnology, China) elicited neutralising antibodies and T cell responses in a dose dependent manner, but was less immunogenic in individuals >55 years of age. A heterologous prime-boost Ad5/Ad26 vectored vaccine schedule (Gamaleya Research Institute, Russia) generated neutralising antibody and cellular responses in adults <60 years of age.

### Summary

Healthy adults aged 18-55, 56-69 or >70 years were randomised to receive either intramuscular ChAdOx1nCoV-19 (at either a low or standard dose) or a control vaccine, MenACWY, in a phase II component of a Phase II/III randomised controlled trial. Analyses were by group allocation in participants who received vaccine. Here, we report the preliminary findings on safety, reactogenicity, and cellular and humoral immune responses. The study is ongoing and is registered at Clinicaltrials.gov, NCT 04400838, and ISRCTN, 15281137.

Local and systemic reactions were more common in the ChAdOx1nCoV-19 groups than the control groups, and similar in nature to previously reported (pain at injection site, feeling feverish, muscle ache, headache), but were less commonly experienced by older adults than younger adults. Eleven serious adverse events (SAEs) occurred during the study period, all of which were considered unrelated to the study vaccines. Total IgG and neutralising antibody responses following the first dose were lower in those who received the low dose vaccine and in older adults, but responses were similar in all groups after the booster dose was administered. Neutralising antibody responses were higher in the boosted groups when compared with the non-boosted groups. T cell responses peaked at day 14 and were similar across all age groups.

ChAdOx1 CoV-19 is better tolerated in older adults than younger adults and has similar immunogenicity across all age groups after a booster dose.

This study is the fourth clinical trial of a vaccine against SARS-CoV-2 tested in an older adult population. The vaccine was safe and well tolerated, with reduced reactogenicity in older adults.

Antibody responses against the SARS-Cov-2 spike protein were induced in all age groups and were boosted and maintained at 28 days post booster vaccination, including those in the over 70-year group. Cellular immune responses were also induced in all age and dose groups, peaking at day 14 post vaccination.

Immunisation with ChAdOx1 nCoV-19 results in development of neutralizing antibodies against SARS-CoV-2 in 100% of participants including older adults, with higher levels in boosted compared with non-boosted groups.

### Introduction

Immunosenescence refers to the gradual deterioration and decline of the immune system brought on by aging. Age-dependent differences in the functionality and availability of T and B cell populations are thought to play a key role in the decline of immune response. Immunosenescence is associated with an increased susceptibility to infection and reduced vaccine responses in older adults and may contribute to the poor outcomes in this age group. There has been a drive to develop vaccines and adjuvant formulations tailored for older adults to overcome this diminished immune response post-vaccination. Assessment of immune responses in older adults is therefore essential in development of COVID-19 vaccines that could protect this vulnerable population.

Preliminary results of a Phase ½ clinical trial of ChAdOx1nCov-19 in adults aged 18-55 years show the vaccine is well tolerated and generates robust neutralising antibody and cellular immune responses against spike glycoprotein(8). Here we present the results of a Phase ⅔ multi-centre study using ChAdOx1nCOV-19 at 2 different doses, in adults including those over 70 years, and in a 1 or 2 dose regimen.

### Methods and Procedures

In the previous Phase I/II study, a single standard dose of 5 × 10¹⁰ vp ChAdOx nCov-19 was used, based on previous experience with a ChAdOx1MERS construct(9). In this study we assessed a lower dose of 2.2 × 10¹⁰ vp with a standard dose of 3.5-6.5 × 10¹⁰ vp in adults of different age cohorts. Due to the need to rapidly produce large numbers of doses of GMP-manufactured vaccine to allow timely enrolment into the Phase II/III clinical trial, two different batches of vaccine: manufactured and vialed by Advent .r.I. (Pomezia, Italy) and manufactured by COBRA Biologics Ltd (Keele, UK) and vialed by Symbiosis, were used in this study. Both were manufactured according to Good Manufacturing Practice, as described in the Investigational Medicinal Product Dossier and approved by the regulatory agency in the UK, the MHRA.

Analytical comparability assessment of the batches indicates that the batches are comparable.

ChAdOx1nCoV-19 was administered as a single or two-dose regimen (4-6 weeks apart) at a either a low dose (LD) of 2.2x10¹⁰ vp or a standard dose (SD) of between 3.5 and 6.5 × 10¹⁰ viral particles, measured by either UV spectroscopy (Symbiosis) or qPCR (Advent). It was administered as a single intramuscular injection into the deltoid, according to specific study SOPs. The MenACWY vaccine was provided by the UK Department of Health and Social Care and administered as per summary of product characteristics at the standard dose of 0.5 mL: https://www.medicines.org.uk/emc/medicine/26514#gref.

Participants from each group were instructed to complete a diary card to record and assess severity of adverse events. All groups were required to report both solicited and unsolicited AEs for 7 days, with subgroups also reporting unsolicited AEs for a full 28 days. Protocol defined solicited local adverse events included injection site pain, tenderness, warmth, redness, swelling, induration, and itch and solicited systemic adverse events included malaise, muscle ache, joint pain, fatigue, nausea, headache, chills, feverishness (ie, a self-reported feeling of having a fever), and objective fever defined as an oral temperature of 38° C. or higher.

Severity of adverse events was graded with the following criteria: mild (transient or mild discomfort for <48 h, no interference with activity, and no medical intervention or therapy required), moderate (mild to moderate limitation in activity, and no or minimal medical intervention or therapy required), severe (marked limitation in activity and medical intervention or therapy required), and potentially life-threatening (requires assessment in emergency department or hospitalisation). Unsolicited adverse events were reviewed for causality by two clinicians blinded to group allocation, and events considered to be possibly, probably, or definitely related to the study vaccines were reported. Laboratory adverse events were graded by use of site-specific toxicity tables, which were adapted from the US Food and Drug Administration toxicity grading scale.

All participants in in the 56-69 and 70+ age groups and participants in the 18-55 SD/SD groups had clinical and immunogenicity assessments at 0, 7, 14 and 28 days after their prime and booster vaccinations. Participants in the 18-55 LD/LD group had clinical and immunogenicity assessments at 0 and 28 days after their prime and booster vaccinations.

Cellular responses were assessed using an ex-vivo interferon-y enzyme-linked immunospot (ELISpot) assay to enumerate antigen-specific T cells. Humoral responses at baseline and following vaccination were assessed using a standardised total IgG ELISA against trimeric SARS CoV-2 spike protein, a multiplexed immunoassay (Meso Scale Discovery multiplexed immunoassay [MIA] against spike and receptor binding domain), live SARS-CoV-2 neutralisation assays (Public Health England [PHE] microneutralisation assay [MNA IC₈ₒ]), and a pseudovirus neutralisation assay (Monogram PseudoNA IC₅₀). Neutralising antibody to the ChAdOx1vector was measured using a secreted embryonic alkaline phosphatase-reporter (SEAP) assay which measures the reciprocal of the serum dilution required to reduce in vitro expression of vector-expressed SEAP by 50%, 24 hours post transduction.

### Statistical Analysis

Safety endpoints are described as frequencies (%) with 95% binomial exact CIs. Medians and IQRs are presented for immunological endpoints. Participants were analysed according to the group to which they were randomised. To assess the relationship between responses on different assays, linear regression was used to analyse log-transformed post-baseline values. Statistical analyses were performed using SAS version 9.4 and R version 3.6.1 or later.

### Results

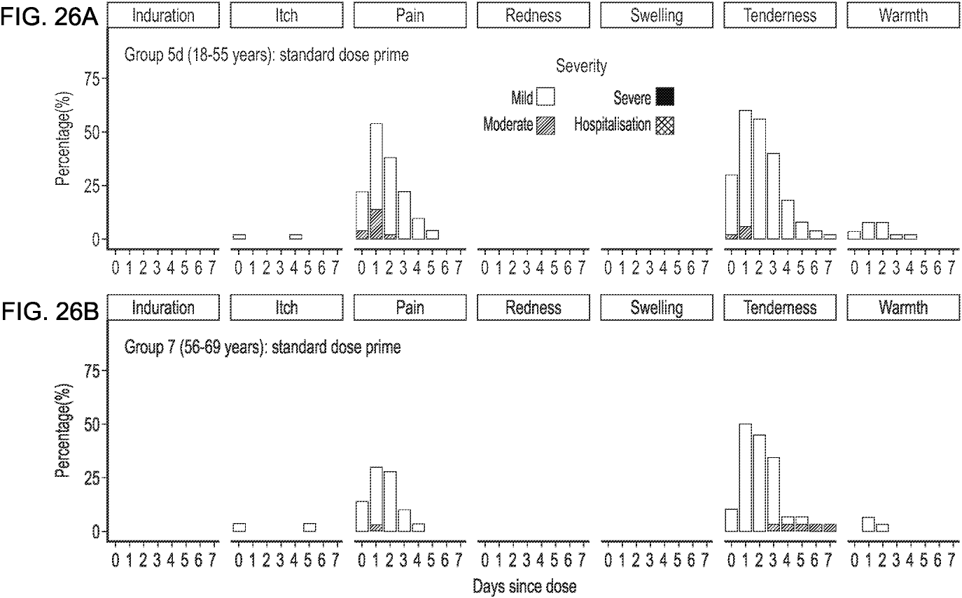

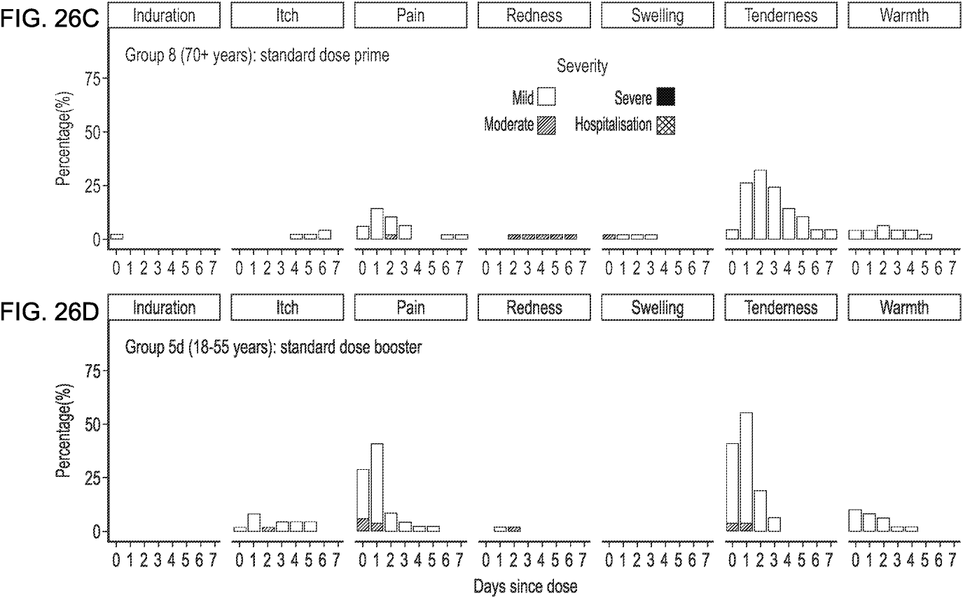

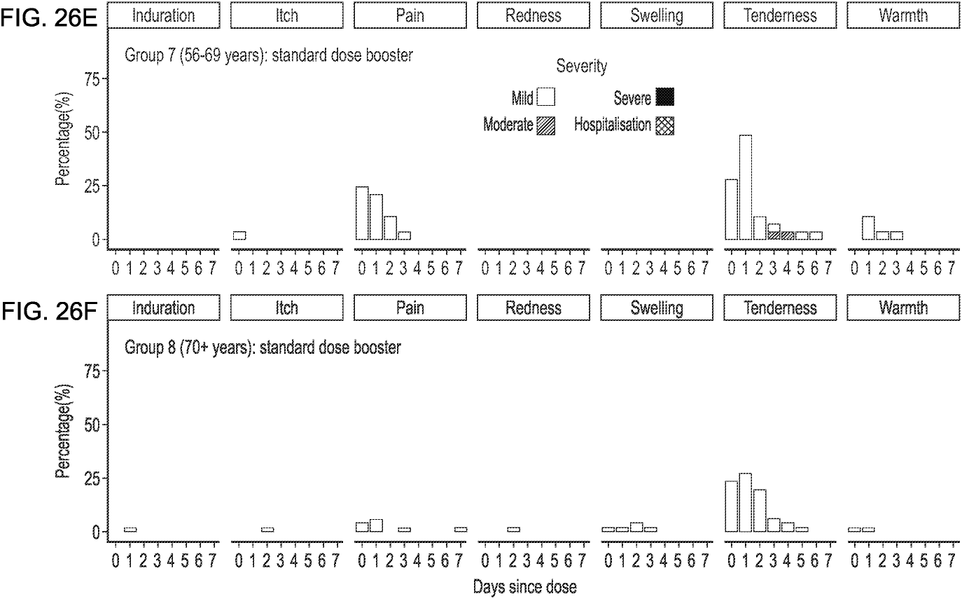

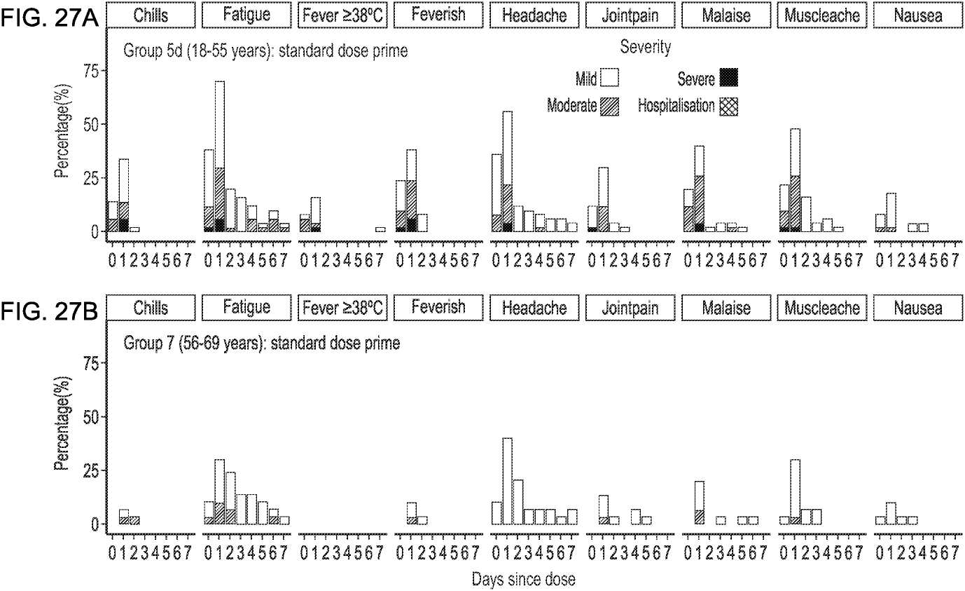

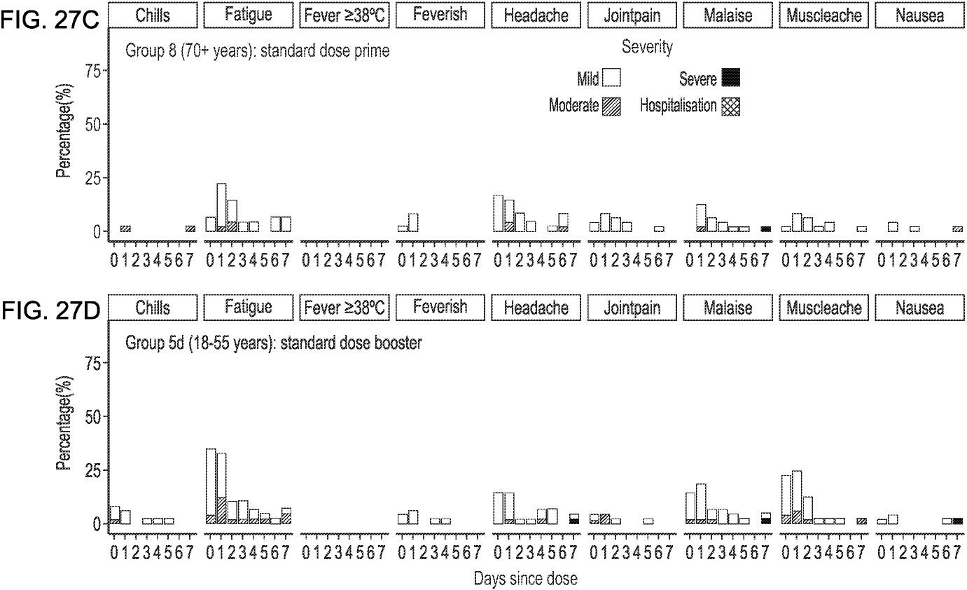

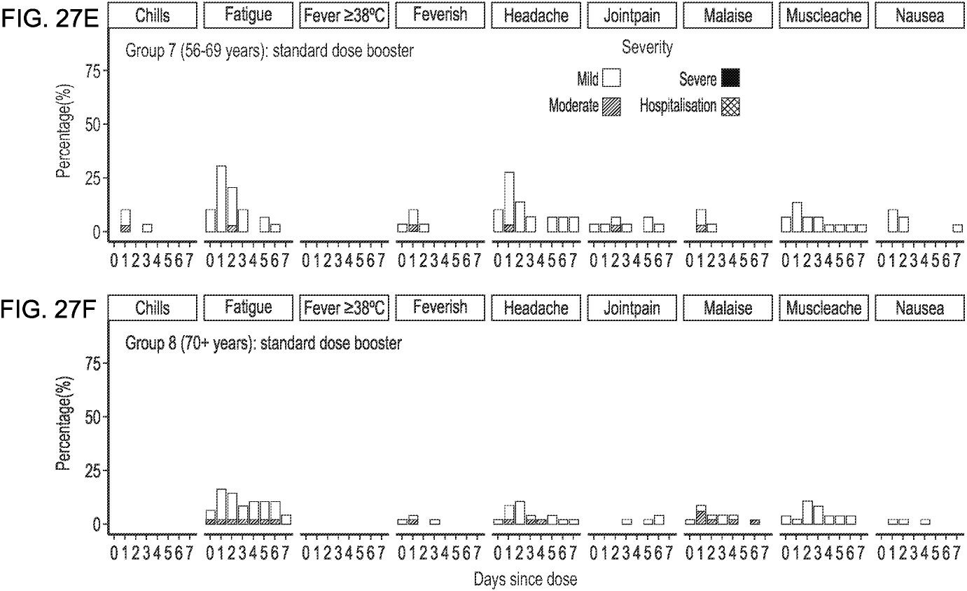

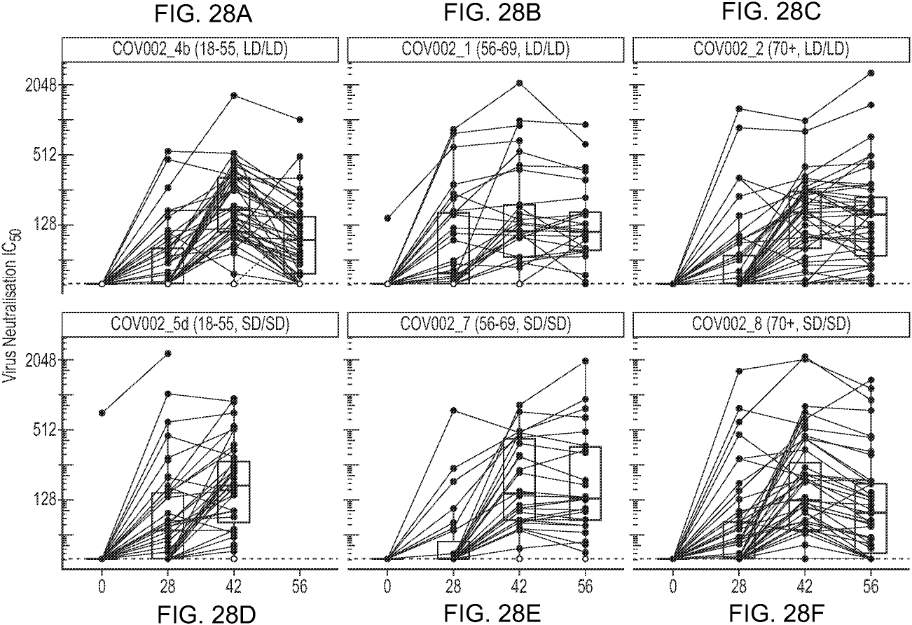

FIGS. 26 to 31 are referred to and show data relating to Example 16.

9869 participants have been recruited to the COV002 trial up to September 2020. 5079 participants have been vaccinated with ChAdOx1nCov-19 and 4790 have received MenACWY. Of these, and reported here, 102 have been enrolled in the 18-55 LD (low dose)/LD group, 60 in 18-55 SD (standard dose)/SD group, 80 in the 56-69 LD/LD group, 80 in 56-69 SD/SD group, 120 in the >70 LD/LD group and 120 in the >70 SD/SD group. All randomised participants were vaccinated. The baseline characteristics of the participants in each group seemed similar between the randomisation allocations.

Injection site pain and tenderness were the most common solicited local adverse reactions and occurred most frequently in the first 48 hours after vaccination. At least one mild to moderate local symptom was reported after prime vaccination with ChAdOx1nCOV-19 by 88.0%, 73.3% and 60.0% of participants in the 18-55 SD/SD group, 56-69 SD/SD group and >70 SD/SD group respectively. Similar proportions of local symptoms were reported after ChAdOx1nCOV-19 booster vaccination with 75.5%, 72.4% and 55.1% of participants in the 18-55 SD/SD group, 56-69 SD/SD group and >70 SD/SD group respectively reporting at least one mild to moderate local symptom. A similar pattern was seen across the age groups for the LD/LD age groups, but with fewer total adverse reactions. Mild to severe local symptoms were experienced by 56.7%, 40.0% and 28.2% of individuals aged 18-55, 56-69 and >70 after prime vaccination with MenACWY and by 86.4%, 36.8% and 20.0% after booster vaccinations with MenACWY respectively.

Fatigue, headache, feverishness and myalgia were the most commonly solicited systemic adverse reactions. At least one moderate to severe systemic symptom was reported after prime vaccination with ChAdOx1nCOV-19 by 86.0%, 76.7% and 64.0% of participants in the 18-55 SD/SD group, 56-69 SD/SD group and >70 SD/SD group respectively. The severity of symptoms was reduced after booster vaccination with ChAdOx1nCOV-19 with only one participant reporting a severe reaction. 65.3%, 72.4% and 42.9% of participants in the 18-55 SD/SD group, 56-69 SD/SD group and >70 SD/SD group respectively reported at least one mild to severe systemic adverse reaction after a ChAdOx1-nCOV19 booster. The incidence of objectively measured fever was low at 26.0% in the 18-55 SD/SD group, and no cases occurring in both the 56-69 SD/SD and >70 SD/SD groups after prime vaccination with ChAdOx1nCOV-19. No participants of any age experienced objective fever after booster vaccination. A similar pattern was seen across the age groups for the LD/LD age groups but with fewer total adverse symptoms. Mild to severe systemic symptoms were experienced by 61.7%, 47.5% and 30.8% of individuals aged 18-55, 56-69 and >70 after prime vaccination with MenACWY and by 67.8%, 31.6% and 25.0% after booster vaccinations with MenACWY respectively.

Unsolicited adverse events in the 28 days following vaccination considered to be at least possibly related to ChAdOx1nCoV-19 were predominantly mild to moderate in nature and resolved within the follow-up period. Laboratory adverse events considered to be at least possibly related to the study intervention were self-limiting and predominantly mild or moderate in severity.

There have been 11 serious adverse events to date, none of which are considered related to the study vaccine.

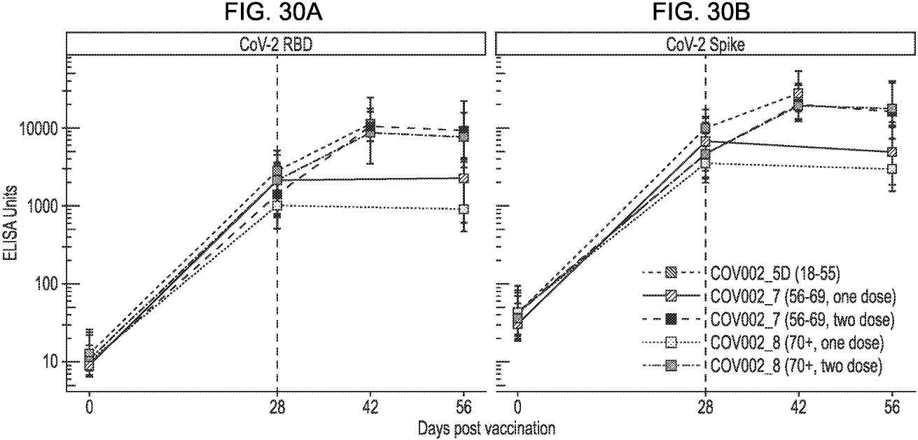

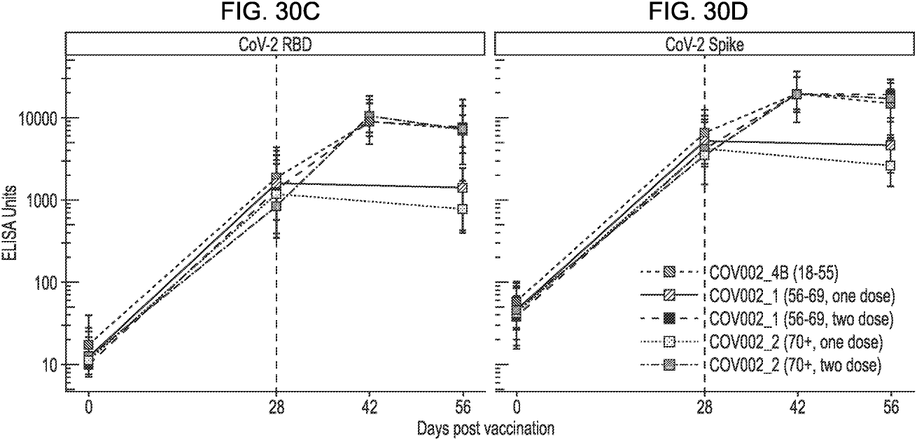

Using a multiplex immunoassay (MIA) which detected total IgG against receptor binding domain and trimeric spike protein, we observed that participants who received a standard prime with ChAdOx1nCOV-19 generally developed higher antibodies titres by day 28 post vaccination than those who received a lower dose (18-55 LD median 6349 Absorbance units [AU], IQR 4438-10640, vs 18-55 SD median 9807 AU, IQR 5847-17220; 56-69 LD median 5032 AU, IQR 2753-9578, vs 56-69 SD median 6693 AU, IQR2814-1370; >70 LD median 4168 AU, IQR 1542-8846, >70 SD median 3454 AU, IQR 1974-11702. By 28 days after booster vaccination, similar antibody titres were seen across all groups regardless of age or vaccine dose (18-55 LD/LD median 15114 AU, IQR 9996-22084, vs 18-55 SD/SD median 27294 AU, IQR 16256-37055; 56-69 LD/LD median 19156 AU, IQR 8918-26348, vs 56-69 SD/SD median 16170 AU, IQR 10233-40353; >70 LD/LD median 16972 AU, IQR 5735-29176, vs >70 SD/SD median 17561, IQR 10717-338177 (see FIG. 30). No antibodies to the spike protein were observed.

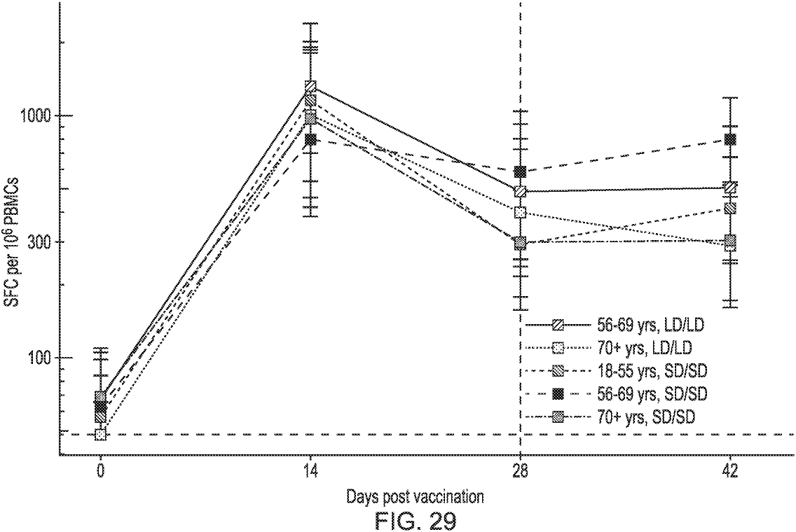

Pseudo-virus neutralization assay titres (Monogram PseudoNA IC₅₀) are shown for low dose and standard dose groups across all ages (FIG. 29). Responses in the low dose groups peaked at day 42, 14 days after the booster vaccination, following the same pattern of response as the ELISA. There was no significant difference in neutralization titres across the age groups receiving the same dose (low dose: 18-55 years median 172, IQR 111-335 vs 56-69 years median 113, IQR 68-192 vs 70+ years median 164, IQR 81-248, p=0.2440). Responses in the standard dose groups followed the same trend, with very similar titres peaking at day 42 (18-55 years median 170, IQR 72-283, 56-69 years median 146, IQR 86-443, vs 70+ SD/SD median 128, IQR 70-296, p=0.6555).

Standard dose recipients had similar titres at day 42 as those who received only low dose vaccines (18-55 y, p= 0.5115, 56-69 y, p= 0.4516, 70+ y, p= 0.7664): FIG. 29, see also table 29S below

TABLE 29S

Summary statistics for Interferon-y ELISpot responses

| Study Group | Visit | N | Single ChAdOx1 | N | Two Dose ChAdOx1 |
|---|---|---|---|---|---|
| 56-69y,LD/LD | 0 | 29 | 57 [48,103] | 30 | 67 [48,109] |
| | 14 | 30 | 1001 [662,1965] | 30 | 1341 [536,2029] |
| | 28 | 28 | 511 [264,790] | 29 | 488 [255,1043] |
| | 42 | | | 29 | 501 [253,905] |
| 70+y LD/LD | 56 | 30 | 207 [111,422] | 29 | 333 [179,505] |
| | 0 | 49 | 48 [48,68] | 41 | 48 [48,71] |
| | 14 | 50 | 1027 [488,2247] | 45 | 1009 [401,1873] |
| | 28 | 48 | 405 [234,716] | 44 | 395 [207, 709] |
| | 42 | | | 44 | 291 [172, 533] |
| | 56 | 35 | 166 [118,379] | 44 | 266 [120, 463] |
| 18-55y SD/SD | 0 | | | 25 | 58 [48,108] |
| | 14 | | | 24 | 1187 [841,2428] |
| | 28 | | | 10 | 292 [178,803] |
| | 42 | | | 23 | 413 [245,675] |
| 56–69 SD/SD | 0 | 25 | 48 [48,65] | 30 | 63 [48,84] |
| | 14 | 21 | 677 [411,1503] | 29 | 797 [383,1817] |
| | 28 | 29 | 335 [162,523] | 30 | 591 [238,922] |
| | 42 | | | 28 | 798 [462,1186] |
| 70+y SD/SD | 0 | 48 | 63 [48,95] | 47 | 73 [48,100] |
| | 14 | 49 | 971 [447,1527] | 48 | 977 [458,1914] |
| | 28 | 48 | 566 [359,979] | 49 | 300 [157,492] |
| | 42 | | | 47 | 307 [161,516] |

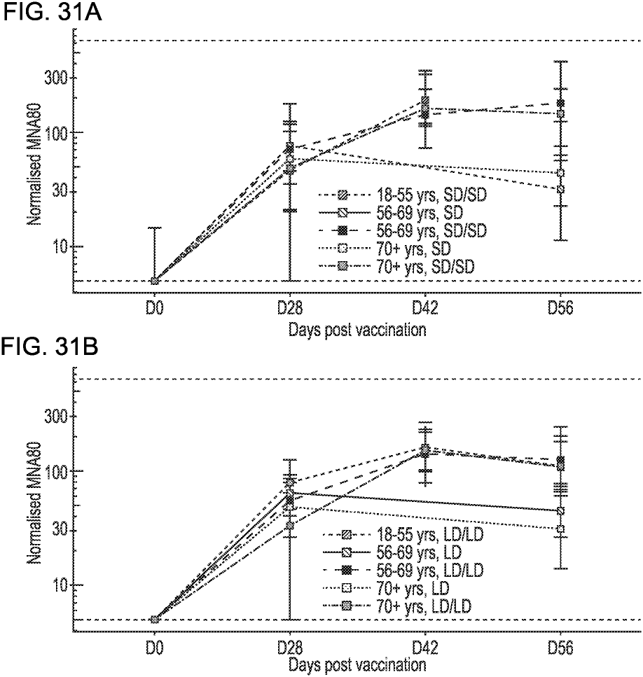

In a live virus microneutralization assay (MNA80) performed at Public Health England, median titres peaked in most boosted groups by day 42. There were no significant difference in neutralization titres between age groups at day 42 (low-dose p=0.946, standard dose p=0.300), and within each age group there was no significant difference in neutralization titres between low and standard dose vaccine recipients at the same timepoint (18-55 y: p=0.3160, 56-69 y: p=0.1468, 70+ y: p=0.745). FIG. 31.

IFN-γ ELISpot responses against SARS-CoV-2 spike protein peaked 14 days after the first dose and did not rise significantly after the second dose (p=0.4622 from paired t-test of day 28 v day 42). ELISpot data were unavailable for the 18-55 LD/LD cohort. In those who received two standard doses, there was a significant difference across age groups with those aged 56-69 years having higher responses at day 42 than other age groups receiving the same vaccine, (median IQR, 413 [245,675] in those 18-55 years, compared with 798 [462, 1186] in those 56-69 years (p=0.0154), and 307 [161,516] in those 70+ years or older (p=5943).

Anti-ChAdOx1 neutralising antibody titres across different age and dose groups increased with the ChAdOx1priming vaccination in all groups to comparable levels but were not increased further after a second vaccine dose at day 28. This was in contrast to the anti-SARS-CoV-2 spike protein antibodies, which were boosted 28 days after the second vaccine dose. Anti-ChAdOx1 neutralising titres at the time of the booster vaccine were negatively correlated with standardised ELISA values 28 days after boost (P=0.0374) but there was no statistically significant correlation between pre-boost Anti-ChAdOx1 neutralising titres and ELISpot responses 14 days after the booster dose (p=0.221).

### Discussion

The robust immune responses obtained in our older adult population were surprising given that a number of studies have demonstrated that declining immune function with age leads to poorer immune responses to vaccines. This holds true for vaccines such as influenza where pre-existing immune memory exists and vaccines that induce primary immune responses such as hepatitis B. Other adenoviral vector platforms against SARS-CoV-2 have either shown reduced immunogenicity in an older age group (although this was a single-dose regimen and so not directly comparable to a prime-boost regimen) or have not yet been tested in an older population.

It is noteworthy that the anti-spike antibody responses in our study increased after booster vaccination at an interval of 1 month but the neutralising anti-vector antibody responses did not. There was also no difference in anti-vector immunity by age.

In the absence of a clear serological correlate of protection from SARS-CoV-2, clinical studies have focussed on neutralising antibodies which confer protection from challenge in animal models. Live neutralisation assays are labour intensive and can only be performed in specialist laboratories under category 3 biological safety conditions. We show here that receptor-binding domain and spike protein MIA titres correlate with neutralising antibody titres across different age groups. This suggests that should neutralising antibody be shown to be protective in humans, MIA could be used for the standardised evaluation of protection afforded by putative vaccine candidates in clinical trials.

### Example 17: Booster Dose of the Viral Vector ChAdOx1nCoV-19 Induces Multifunctional Antibody Responses and is well Tolerated.

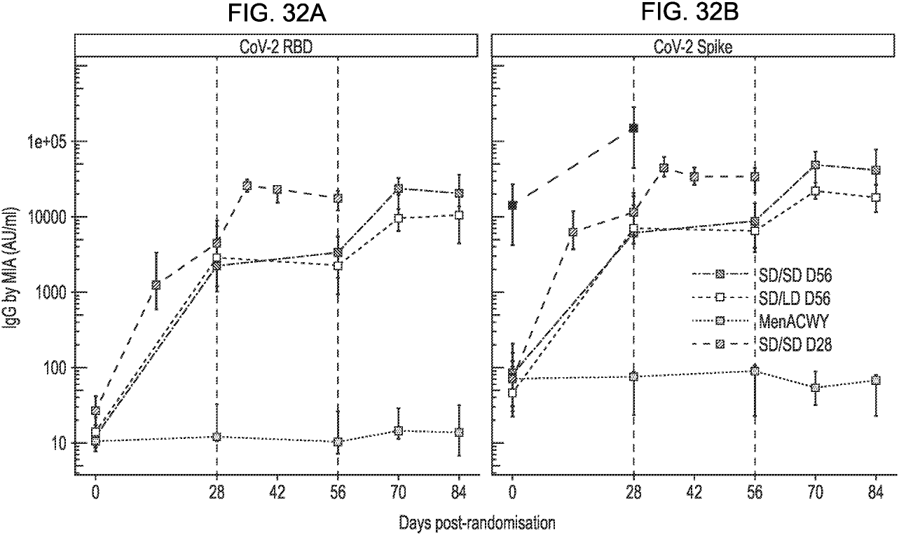

Example 17 relates to a phase I/II randomised controlled trial, it should be read in conjunction with the earlier examples, in particular Examples 11, 15 and 16. FIGS. 32 to 37 show data relating to Example 17.

### Summary

Animal studies suggest the level of neutralising antibody (NAb) against spike protein may correlate with protection, but other antibody functions may be important in preventing infection and control of early cellular invasion by the virus. We have previously reported early immunogenicity and safety of a viral vector coronavirus vaccine, ChAdOx1nCoV-19. Here we demonstrate, in a phase I/II randomised controlled trial, that two doses of ChAdOx1nCoV-19 induce stronger total and neutralising antibody responses than one dose, and that similar responses are seen with a booster at either 1 or 2 months after the first dose, in healthy adults under 55 years of age. Higher doses of vaccine used for boosting induce stronger antibody responses but similar T cell responses, when compared with a dose-sparing half-dose boost. Furthermore, we demonstrate Fc-mediated functional antibody responses (antibody dependent neutrophil/monocyte phagocytosis, complement activation and NK cell activation) that are substantially enhanced by a booster dose of vaccine. We also found that a second dose of vaccine was better tolerated than the first dose, after which reactions were similar to those previously reported. These data supported the two dose vaccine regime that is now being evaluated in phase III clinical trials.

### Introduction

While pre-clinical data suggest that immune responses induced by SARS-CoV-2 infection are important in protection against re-infection in an animal model, the relative contributions of different immune functions were not assessed.

Neutralisation has been the most commonly measured antibody function, as neutralising antibodies have the ability to bind to the surface of the SARS-CoV-2 virion and prevent host cell entry, with the potential to convey sterilising immunity. However other antibody functions may play a role in recovery from, or protection against overt disease.

Here we describe further evaluation of the post-vaccination immune responses of individuals recruited to the phase I/II clinical trial of the ChAdOx1nCoV-19 vaccine. Different boosting schedules and dose sparing of the booster dose were compared, including the quantity and quality of the induced immune response. Individuals received either a single dose or two dose regimen of ChAdOx-1 nCoV-19 at either a 28- or 56-day interval. Cellular immunity is described, and spike-specific antibodies were quantified and functionally characterised using a wide range of assays.

### Results

### MIA and Neutralising Antibody Titres

Anti-spike IgG antibodies to SARS-CoV-2 spike and receptor binding domain (RBD) were measured in a multiplex serology immunoassay. In both cases antibody titres rose after the first vaccination with a further increase after the second. At 14 days after the second dose IgG titres were not significantly different between those who received the booster at 28 days (GMT 35990, 95% CI 24408, 53068) or at 56 days (SD/SD D56: GMT 44485, 95% CI 31714, 62400, p=0.426 and SD/LD D56: GMT 25667, 95% CI 18814, 35015, p=0.250). However, those who received a half-dose boost had lower titres 14 days post-boost than those who received the standard dose (p=0.020). Similar findings were seen for anti-spike and anti-RBD IgG using MIA (FIG. 32, Table 17S2 below). An additional 30 participants were seropositive at the time of receiving a single .

TABLE S2

Anti-spike and anti-RBD IgG by multiplex immunoassay

| Visit | N | MenACWY | | N | SD/SD D28 | | N | SD/SD D56 | | N | SD/LD D56 | |
|---|---|---|---|---|---|---|---|---|---|---|---|---|
| | | GMC (95% CI) | Median [IQR] | | GMC (95% CI) | Median [IQR] | | GMC (95% CI) | Median [IQR] | | GMC (95% CI) | Median [IQR] |
| Anti-Spike IgG | | | | | | | | | | | | |
| 0 | 10 | 58 (27, 124) | 72 [24, 131] | 10 | 141 (24, 809) | 71 [46, 249] | 20 | 89 (39, 204) | 84 [22, 178] | 32 | 52 (36, 76) | 45 [31, 87] |
| 14 | | | | 10 | 7040 (2114, 23451) | 6358 [3588, 12840] | | | | | | |
| 28 | 10 | 49 (24, 101) | 74 [18, 88] | 10 | 13843 (5809, 32988) | 11453 [5775, 20988] | 19 | 8611 (4647, 15956) | 6185 [3985, 18973] | 32 | 7410 (5280, 10400) | 6866 [4203, 14222] |
| 35 | | | | 10 | 51762 (30279, 88489) | 44205 [33772, 66548] | | | | | | |
| 42 | | | | 9 | 35990 (24408, 53068) | 34081 [26583, 45472] | | | | | | |
| 56 | 10 | 56 (26, 122) | 93 [20, 110] | 10 | 34175 (20915, 55842) | 33307 [20281, 43775] | 20 | 7722 (4759, 12532) | 8790 [3565, 15437] | 32 | 5191 (3625, 7435) | 6038 [3304, 8119] |
| 70 | 10 | 53 (29, 96) | 55 [27, 94] | | | | 19 | 44485 (31714, 62400) | 48979 [28038, 83258] | 29 | 25667 (18814, 35015) | 21660 [14953, 45349] |
| | | | | | | | | P=0.4263 (t test vs SD/SD D28 group @ day 42) | | | P=0.2504 (t test vs SD/SD D28 group @ day 42) | |
| | | | | | | | | | | | P=0.0201 (t test vs SD/SD D56 group @ day 70) | |
| 84 | 10 | 48 (23, 101) | 67 [20, 80] | | | 19 | 40176 58027) | (27816, | 41927 [21197, 85118] | 31 | 15761 (7688, 32310) | 17965 [11238, 47038] |

Anti-RBD IgG

| | | | | | | | | | | | | |
|---|---|---|---|---|---|---|---|---|---|---|---|---|
| 0 | 10 | 16 (9, 28) | 10 [9, 33] | 10 | 47 (9, 255) | 27 [16, 42] 20 | 18 (9, | 36) | 12 [8, 24] | 32 | 15 (11,20) | 13 [9, 21] |
| 14 | | | | 10 | 1584 (331, 7573) | 1274 [396, 3612] | | | | | | |
| 28 | 10 | 17 (10,29) | 12 [11,33] | 10 | 5356 (1959, 14641) | 4494 [2245, 9729] 19 | 2729 5599) | (1330, | 2252 [925, 8231] | 32 | 1823 (1154, 2879) | 2521 [865, 4099] |
| 35 | | | | 10 | 29478 (18735, 46381) | 25572 [20912, 31747] | | | | | | |
| 42 | | | | 9 | 21992 (15400, 31405) | 23576 [15516, 24870] | | | | | | |
| 56 | 10 | 15 (8, 28) | 10 [6, 27] | 10 | 19764 (12476, 31311) | 17670 [12041, 22502] 20 | 3125 5302) | (1842, | 3352 [1548, 5583] | 32 | 1459 (931, 2287) | 1536 [708, 2969] |
| 70 | 10 | 18 (10,32) | 14 [10, 30] | | | 19 | 21045 29925) | (14800, | 24181 [11779, 34420] | 29 | 10565 (7343, 15200) | 9030 [6467, 19581] |
| 84 | 10 | 15 (7, 29) | 14 [6, 35] | | | | 19 | 20627 (14327, 29696) | 20373 [13502, 36658] | 31 | 7423 (3663, 15044) | 9260 [4187, 21572] |

vaccination, and anti-spike IgG titres increased 10-fold after vaccination. FIG. 32 shows time course of IgG responses are shown for three ChAdOx1nCoV-19 prime-boost groups; SD/SD: two standard doses administered either 28 or 56 days apart, SD/LD: standard dose prime followed by low dose boost 56 days apart and for two doses of MenACWY comparator vaccine. Dotted lines show timepoints at which boosting occurred. Plot shows median and interquartile range. AU/ml = Arbitrary units/ml. Left panel anti-RBD (Receptor Binding Domain) responses. Right panel anti-Spike (SARS-COV-2 spike protein) responses. Dashed line indicates responses in 30 participants who received only one dose of ChAdOx1nCoV-19 and were seropositive at baseline (seropositivity threshold defined as anti-spike IgG > 1000 AU/ml).

Neutralising antibodies were assayed using a microneutralisation assay reporting the reciprocal of the serum dilution required to reduce live SARS-CoV-2 infection of single cells by 80% (MNA₈₀). NAb were induced following prime vaccinations and significantly increased after a booster dose in all 3 ChAdOx1nCoV-19 groups. Median NAb titres at 14 days post boost were 136 (IQR 115, 241) for SD/SD D28, 169 (IQR 134, 372) for SD/LD D56, and 315 (IQR 224, 531) for SD/SD D56.

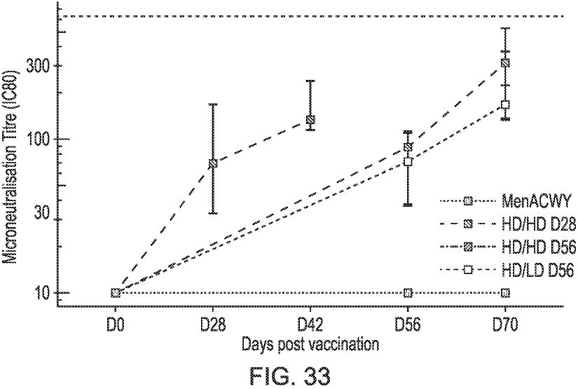

FIG. 33 shows Timecourse of Microneutralisation titre at IC₈₀ is shown for three ChAdOx1 nCoV-19 prime-boost groups; SD/SD: two standard doses administered either 28 or 56 days apart, SD/LD: standard dose prime followed by low dose boost 56 days apart and for two doses of MenACWY comparator. Error bars show medians and inter-quartile ranges.

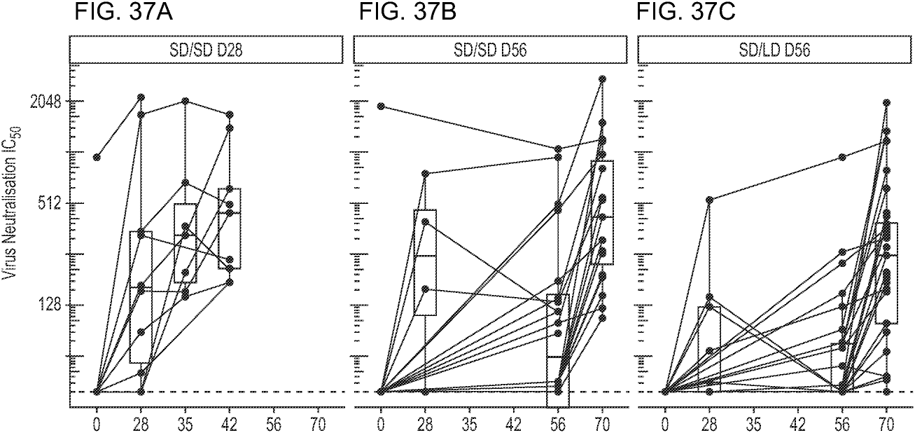

No NAb activity was observed in the MenACWY group. Neutralising antibodies were also determined in a pseudovirus neutralisation assay reporting IC₅₀. Median NAb titres on the pseudovirus assay at 14 days post boost were 451 (IQR 212, 627) for SD/SD D28, 253 (IQR 100, 391) for SD/LD D56 and 424 (IQR 229, 915) for SD/SD D56 (see FIG. 37, and Table 17S3, below).

TABLE 17S3

Neutralising antibody measured in pseudovirus assay (Monogram IC50)

| Visit | N | SD/SD D28 Median [IQR] | N | SD/SD D56 Median [IQR] | N | SD/LD D56 Median [IQR] |
|---|---|---|---|---|---|---|
| 0 | 10 | 40 [40, 40] | 20 | 40 [40, 40] | 32 | 40 [40, 40] |
| 28 | 10 | 163 [61, 346] | | | | |
| 35 | 7 | 334 [179, 529] | | | | |
| 42 | 9 | 451 [212, 627] | | | | |
| 56 | | | 20 | 68 [40, 151] | 32 | 40 [40, 76] |
| 70 | | | 19 | 424 [229, 915] | 29 | 253 [100, 391] |

### Antibody Class and Subclass

Antibody classes and subclasses within the anti-spike response were determined. Based on previous isotype analysis after ChAdOx1 nCoV-19 vaccination (D. Barouch et al. 2018), we focused on measuring IgA and IgM titres of anti-SARS-CoV-2 spike antibodies. Vaccination with ChAdOx1 nCoV-19 increased the IgM and IgA titres with a peak response measured 28 days after prime for IgM. There was no difference in the response measured 14 days after SD or LD boost, and the post-boost response was of a similar magnitude to SD/SD with a 28 day interval.

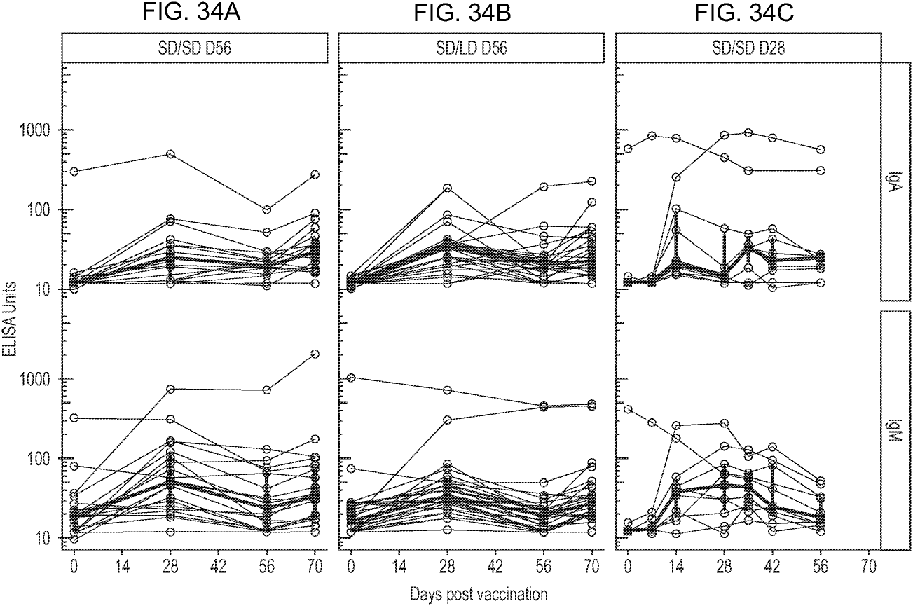

FIG. 34 shows SARS-CoV-2 spike-specific immunoglobulin isotype responses induced by prime-boost regimens of ChAdOx1 nCoV-19. Volunteers received a standard dose (SD) of ChAdOx1 nCoV-19 at day 0 followed by a second vaccination with SD at day 56 (left panel) or low dose (LD) at day 56 (middle panel) or SD at day 28 (right panel) of ChAdOx1 nCoV-19. SARS-CoV-2 spike trimer-specific IgA and IgM responses were quantified by ELISA and expressed as ELISA units. Solid lines connect samples from the same participant. Bold solid lines show median with IQR.

Serum samples that were IgG positive 28 days after vaccination were assayed for anti-spike IgG subclasses. IgG1 and IgG3 responses were readily detectable at day 28 and were at a similar level on day 56 in regimens with a 56 day interval. Following booster vaccination, the median IgG1 response did not increase in those who received the standard dose regimen with a 28 day interval, although this may be limited by the small group size. IgG1 responses did increase 14 days after a SD or LD boost in regimens with a 56 day interval, with no measured difference due to dose. IgG3 responses were increased following booster vaccination across all three regimens regardless of interval or dose. The response was predominantly IgG1 and IgG3, with low levels of IgG2 and IgG4.

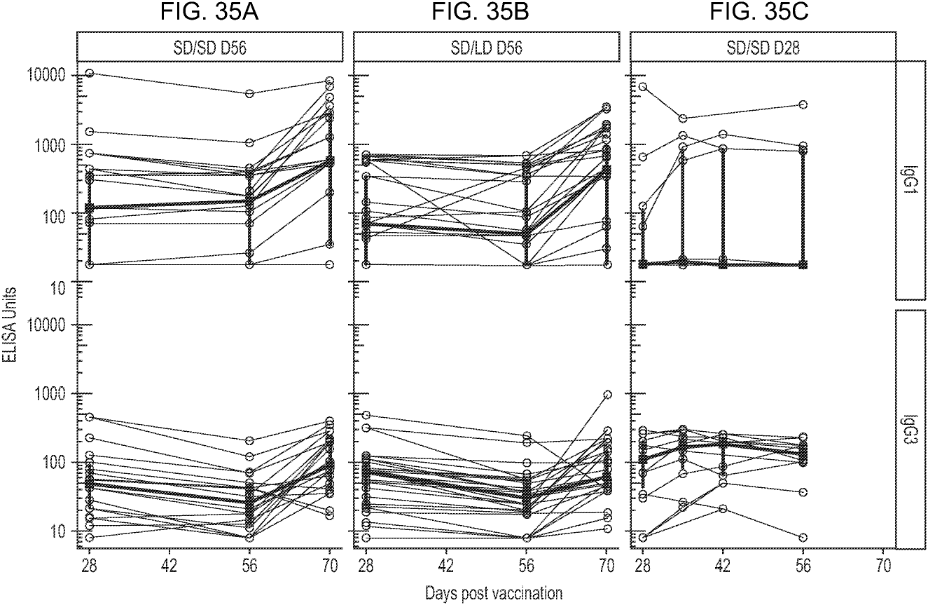

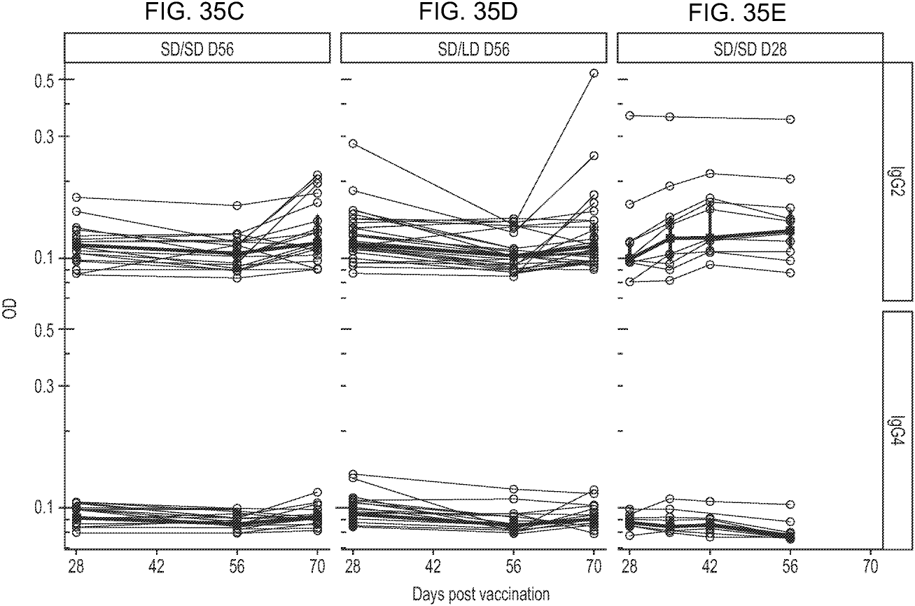

FIG. 35 shows SARS-CoV-2 spike-specific IgG subclass responses induced by prime-boost regimens of ChAdOx1nCoV-19. Volunteers received a standard dose (SD) of ChAdOx1 nCoV-19 at day 0 followed by a second vaccination with SD at day 56 (left panel) or low dose (LD) at day 56 (middle panel) or SD at day 28 (right panel) of ChAdOx1 nCoV-19. Volunteers with measurable SARS-CoV-2 spike-specific IgG at day 28 were assayed for IgG subclasses. SARS-CoV-2 spike-specific antibody responses were quantified by ELISA . IgG1 and IgG3 responses were expressed as ELISA units and IgG2 and IgG4 responses expressed as OD at 405 nm. Solid lines connect samples from the same participant. Bold solid lines show median with IQR.

This predominant Th1-type IgG response is in agreement with other studies investigating adenoviral vectored vaccine priming in humans. These analyses highlight the similarity in antibody response induced after ChAdOx1 nCoV-19 vaccination regardless of interval or booster dose.

### Antibody Functionality

Antibody function was explored further to determine the ability of antibodies induced by vaccination to support antibody-dependent monocyte phagocytosis (ADMP), and neutrophil phagocytosis (ADNP). Both functions were induced by the first vaccination and substantially increased by the second, with a trend towards a larger increase when the interval between the doses was 56 rather than 28 days, and when the booster dose was SD rather than LD (see Table 17S4, below).

TABLE 17S4

Antibody dependent monocyte phagocytosis (ADMP), neutrophil phagocytosis (ADNP), complement deposition (ADCD) and natural killer cell activation (ADNKA) in trial participants, convalescent plasma, and pre-pandemic plasma

| Group | Timepoint | | ADNP | | ADMP | | ADCD (log10 units) | | ADNKA |
|---|---|---|---|---|---|---|---|---|---|
| | | N | Median [IQR] | N | Median [IQR] | N | Median [IQR] | N | Median [IQR] |
| MenACWY | 0 | 2 | 0.02 [0.01, 0.02] | 2 | 0.17 [0.14, 0.2] | 2 | 2.27 [2.14, 2.40] | 2 | 0.58 [0.53, 0.63] |
| MenACWY | 56 | 3 | 0.02 [0.02, 0.03] | 3 | 0.11 [0.1, 0.19] | 3 | 1.99 [1.98, 2.31] | 2 | 0.87 [0.80, 0.93] |
| MenACWY | 70 | 3 | 0.02 [0.01, 0.02] | 3 | 0.19 [0.17, 0.24] | 3 | 2.68 [2.61, 2.81] | 2 | 1.63 [1.45, 1.82] |
| SD/LD D56 | 0 | 20 | 0.01 [0.01, 0.02] | 24 | 0.13 [0.1, 0.18] | 11 | 2.53 [2.41, 2.73] | 22 | 0.4 [0.22, 0.94] |
| SD/LD D56 | 56 | 24 | 0.07 [0.04, 0.18] | 24 | 0.29 [0.21, 0.38] | 12 | 2.84 [2.53, 2.91] | 22 | 2.03 [1.43, 3.13] |
| SD/LD D56 | 70 | 23 | 0.68 [0.32, 0.77] | 22 | 0.73 [0.49, 0.96] | 12 | 3.2 [3.01, 3.56] | 22 | 5.29 [3.61, 6.13] |
| SD/SD D28 | 0 | | | | | | | 4 | 1.17 [0.21, 2.95] |
| SD/SD D28 | 14 | 8 | 0.09 [0.02, 0.14] | 9 | 0.23 [0.19, 0.41] | | | 8 | 2.35 [2.15, 4.73] |
| SD/SD D28 | 28 | 9 | 0.11 [0.03, 0.4] | 9 | 0.3 [0.13, 0.61] | | | 9 | 3 [2.06, 5.66] |
| SD/SD D28 | 35 | 10 | 0.67 [0.55, 0.73] | 10 | 0.89 [0.64, 1.01] | | | 10 | 5.69 [5.12, 7.42] |
| SD/SD D28 | 42 | 7 | 0.76 [0.71, 0.88] | 9 | 1.02 [0.82, 1.05] | | | 8 | 3.96 [3.44, 5.36] |
| SD/SD D28 | 56 | 9 | 0.67 [0.41, 0.8] | 10 | 0.73 [0.54, 0.97] 1 | | | 9 | 3.91 [3.05, 5.25] |
| SD/SD D56 | 0 | 16 | 0.02 [0.01, 0.03] | 18 | 0.18 [0.12, 0.31] | 10 | 2.6 [2.37, 2.76] | 19 | 0.96 [0.12, 1.32] |
| SD/SD D56 | 56 | 18 | 0.08 [0.04, 0.26] | 19 | 0.32 [0.25, 0.38] | 10 | 3.01 [2.74, 3.57] | 19 | 2.27 [1.31, 3.21] |
| SD/SD D56 | 70 | 18 | 1.02 [0.79, 1.17] | 19 | 1.14 [0.75, 1.42] | 10 | 4.83 [4.15, 5.4] | 19 | 5.78 [4.33, 7.7] |
| Pre-2020 | | 14 | 0 [0, 0.01] | 19 | 0.18 [0.12, 0.19] | | | 16 | 1.4 [0.78, 2.69] |
| Conv - Mild | | 33 | 0.31 [0.1, 0.62] | 36 | 0.39 [0.23, 0.65] | 24 | 3.88 [3.33, 4.49] | 21 | 5.31 [2.97, 8.77] |
| Conv - Mod/Sev | | 6 | 0.96 [0.92, 1.03] | 6 | 1.28 [1.23, 1.33] | 6 | 5.53 [5.41, 5.57] | 4 | 6.57 [4.93, 9.63] |
| Conv - Asymp | | 6 | 0.34 [0.2, 0.58] | 6 | 0.4 [0.23, 0.56] | 7 | 3.75 [3.01, 3.8] | 3 | 1.21 [0.86, 8.94] |

In comparison with serum and plasma samples taken from convalescent COVID patients between 28 and 91 days after a positive PCR test, both ADMP and ADNP were higher in the vaccinated group after the second dose. Serum samples taken prior to 2020 were negative in both assays and there was no change in these functions in participants who received the MenACWY vaccine.

Antibody-dependent complement deposition (ADCD) was also induced by prime vaccination and significantly increased following booster doses at D56. Higher median fluorescence intensity (MFI) were observed in recipients of a standard booster dose compared to those receiving half dose.

The capacity of the ChAdOxl nCoV-19 vaccine to induce antibody-dependent NK cell activation (ADNKA) in humans was also explored, and reported as the capacity to trigger CD107a expression. Results demonstrate that single dose ChAdOx1 nCoV-19 induced low ADNKA responses which were boosted by the second dose given either at day 28 or 56. The dose used for boosting at day 56 had no impact on the resulting ADNKA measured at day 70 (SD/SD 56 median 5.78 IQR 4.33, 7.7, SD/LD 56 median 5.29 IQR 3.61, 6.13), whereas ADNKA measured at day 42 after boosting at D28 was lower (median 3.96 IQR 3.44, 5.36). The responses observed after two doses of vaccines were within similar range to those detected in a cohort of 21 convalescent COVID patients (median 5.31 IQR 2.97, 8.77) whereas no change was detected after MenACWY vaccination.

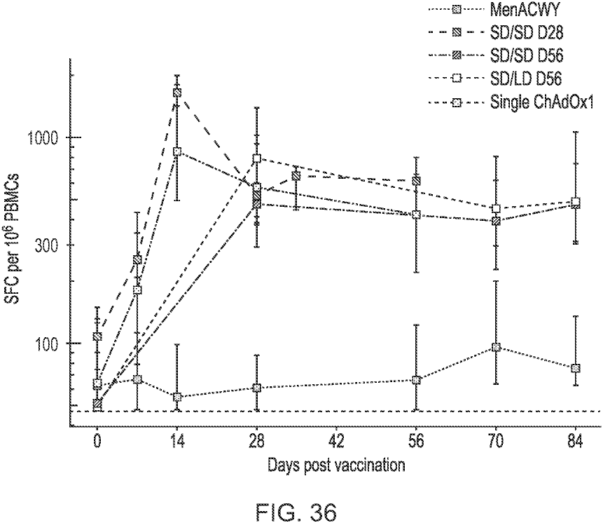

FIG. 36 shows IFNγ ELISpot response to peptides spanning the SARS-CoV-2 spike vaccine insert after vaccination with ChAdOx1 nCoV-19. The total ex vivo T cell response to the SARS-CoV-2 spike vaccine insert encoded within the vaccine is shown over time (IFNγ ELISpot; spot forming cells per 10⁶ PBMC; calculated by summing the responses to peptide pools corrected for background; materials and methods). Response is shown as median with interquartile range per vaccination regimen; Single ChAdOxl: one standard dose of ChAdOx1 nCoV-19, SD/SD: two standard doses of ChAdOx1 nCoV-19, SD/LD: standard dose prime and low-dose boost. Participants received a booster dose of ChAdOx1 nCoV-19 at day 28 (SD/SD D28) or at day 56 (SD/SD D56, SD/LD D56. The LLD is 48 SFC and is denoted by a dotted line.

### Discussion

We present strong evidence that boosting enhances both the titre and the functionality of the antibody response, which strengthens the preliminary findings on immunogenicity of ChAdOx1 nCoV-19. Additionally, we show clear evidence that a booster dose is less reactogenic than the first dose. The data presented here were key to supporting the decision to change from a one dose to a two dose regimen for the phase III trials of ChAdOx1-nCoV19 which are now underway.

Tolerability of vaccines is important for public acceptance, and the expected reactogenicity profile of the different products which may be used to control SARS-CoV-2 has to be fully characterised and communicated to future vaccine recipients before deployment. In earlier examples there was a trend towards lower reactogenicity following the second dose of ChAdOx1 nCoV-19 in a small number of vaccinees. That finding is now confirmed here in a larger cohort in whom second doses were consistently less reactogenic than the first, regardless of dose interval. The observation of reduced second dose reactogenicity is in contrast to reported profiles of two mRNA vaccines for COVID19 and a protein-adjuvant vaccine technology, in which, though generally well tolerated, reactogenicity increased with the second dose. This phenomenon is noteworthy since it is conceivable that additional doses may be required in the future to sustain immunity. Schedules that mix the different vaccine technologies in heterologous prime boost regimens may maximise immunogenicity, whilst limiting reactogenicity, and could result in innovative strategies that harness the strengths of the different technologies.

There was no association between reactogenicity and presence or absence of antibodies to either SARS-CoV-2 or ChAdOx1 at the time of vaccination. This is an important finding when considering extended use of the vaccine post licensure, when antibody screening will not be performed prior to vaccination and a variable proportion of the population will already have been exposed to SARS-CoV-2. Antibodies against the ChAdOx1 vector are induced by the first vaccination but do not prevent boosting and are not further increased by the second vaccination with either a 4 week or 8 week interval.

Here we see induction of IgA and IgM in addition to antibody-dependent functional activities ADMP, ADNP, ADCD and ADNKA, and are stronger after a second dose.

Whilst the titre of neutralising antibodies capable of preventing cellular invasion has emerged as the strongest correlate of protection in pre-clinical SARS-CoV-2 vaccine studies, non-neutralising functional activities are increasingly recognised as important mediators of viral control, working in tandem with CD8+ T cells to kill virally infected cells in the host (Excler et al. 2014; DiLillo et al. 2014). In preclinical studies of SARS-CoV-2 vaccination, Fc-mediated antibody functions including ADCD and ADNKA correlated with protection against infection following viral challenge, and, in combination with neutralising antibodies, enhanced the ability to distinguish fully protected rhesus macaques from those which become infected (Atyeo et al. 2020; Yu et al. 2020; Mercado et al. 2020). In this study, ADNP, ADMP and ADNKA responses induced by ChAdOx1 nCoV-19 were in the same range or higher than that observed a set of samples from convalescent individuals collected more than one month after disease. A study comparing SARS-CoV-2 humoral responses in patients who survived with those who died, found S-specific humoral responses, including Fc-mediated functional activities, were enriched among individuals who survived, while nucleocapsid-specific responses were elevated in deceased individuals (nucleocapsid antibodies are not induced by ChAdOx1 nCOV-19) (Atyeo et al. 2020). These data suggest that functional antibodies against the S protein may be important in resolving disease.

### Methods

### Antibody-Dependent Natural Killer Cell Activation Assay (ADNK)

To assess the antigen-specific antibody-dependent NK cell activation (ADNKA), 96-well Nunc Maxisorp ELISA plates (Thermo Fisher) were coated with recombinant SARS-CoV-2 spike protein at 2.5 ug/ml in carbonate/bicarbonate solution (Sigma Aldrich) for 16 hours at 4° C. Plates were washed with phosphate buffer saline (PBS) and blocked with 5% BSA in PBS. Serum and plasma samples were plated undiluted in duplicate. Following incubation for two hours at 37C, the plates were washed and 10⁵ natural killer cells (cell line NK92.05-CD16, American Type Culture Collection, and described in Binyamin et al. (Binyamin et al. 2008) were added per well in the presence of Brefeldin A (10 ug/ml, Sigma Aldrich), Golgi Stop (BD Biosciences) and CD107a (PE, clone H4A3, BD Biosciences). A sample of cells were separately stained with CD56 (BV786, clone NCAM16, BD Biosciences) and CD16 (AF594, clone GRM-1, Santa Cruz Biotechnology) in order to verify consistent expression of CD16. After 5 hours incubation, cells were transferred to V-bottom plates and stained for FACS analysis. Live NK cells were identified by fixable LIVE/DEAD staining (R780, BD Biosciences). Cells were fixed and data acquired using a BD Fortessa. Percentages of CD107a+NK cells relative to control wells with spike protein and blocking buffer only were determined in FlowJo software (version 10.7.1). A pre-pandemic pool of three donors and a pool of six hospitalised SARS-CoV-2 infected individuals were plated in triplicate on each plate, for quality control of each assay.

### Bead Coupling for ADMP and ADNP Assays

Red fluorescent (580/605) NeutrAvidin-labelled microspheres (Thermo Fisher, F-8875) were freshly coupled to biotinylated SARS-CoV2 spike protein for each assay. Spike protein (at a concentration of 0.388 µl/mL) was coupled to the beads at a 3:1 ratio and incubated for 2 hours at 37° C. Beads were washed twice with 0.1% BSA and diluted 100-fold in 0.1% BSA 10 µl was added to each well in the ADNP and ADMP assays.

### Antibody Dependent Neutrophil Phagocytosis (ADNP)

The ADNP assay is based on a previously described protocol (Karsten et al. 2019) with some modifications. Whole donor blood, collected in sodium heparin tubes, was treated with Ammonium-Chloride-Potassium (ACK) lysing buffer (Thermo Fisher, A1049201) for 5 minutes followed by centrifugation to collect white blood cells. Cells were washed with DPBS (Sigma, D8537), counted and adjusted to 2.5×10⁵ cells/mL in media consisting of Roswell Park Memorial Institute (RPMI) 1640 medium (Sigma, R5886) supplemented with 100 U/mL penicillin/streptomycin (Sigma, P4458) and 20 mmol/L L-glutamine (Sigma, G7513).

Serum diluted 100x in RPMI was added to antigen-coupled beads in a 96-well plate and incubated for 2 hours at 37° C. All samples were assayed in duplicate and each plate contained 2 quality control (QC) samples in addition to appropriate negative controls. Wells were washed with DPBS and a total of 500,000 white blood cells were added per well followed by a further 1 hour incubation at 37° C. Cells were then stained using a cocktail of CD3 Alexa700 (BD, 557943), CD14 APC-Cy7 (BD, 557831) and CD66b Pacific Blue (Biolegend, 305112) and incubated for 15 minutes at room temperature in the dark. Following washing and fixation using 4% paraformaldehyde (PFA, Santa Cruz Biotechnology, SC-281692), the proportion of cells containing fluorescently-labelled beads was ascertained using flow cytometry (BD, Fortessa X20).

Data were analysed with Flowjo (BD, Version 10), using a gating strategy to select neutrophils. Normalised phagocytic scores were calculated by multiplying the percentage of bead-positive cells by the mean fluorescence intensity (MFI) of the beads within these cells and normalising against a QC sample set to 1. As multiple plates were run during an experiment, plates failed if any of the QC sample averages fell outside of the mean +/- 2 SD range of that particular QC across plates. In addition, samples were excluded from further analysis if the replicates showed a coefficient of variation (CV) over 25%. The data in the current paper are all derived from one experiment.

### Antibody Dependent Monocyte Phagocytosis (ADMP)

The ADMP assay is based on a previously described protocol (Ackerman et al. 2011) with some modifications. Briefly, a human monocytic THP-1 cell line (ATCC) was grown and maintained using supplier instructions. Serum was diluted 4000x in RPMI and was added to antigen-coupled beads in a 96-well plate and incubated for 2 hours at 37° C. All samples were assayed in duplicate and each plate contained 2 quality control (QC) samples in addition to appropriate negative controls. At the end of the 2 hours incubation period, wells were washed with RPMI and 250,000 THP-1 cells diluted in media consisting of Roswell Park Memorial Institute (RPMI) 1640 medium (Sigma, R5886) supplemented with 100 U/mL penicillin/streptomycin (Sigma, P4458) and 20 mmol/L L-glutamine (Sigma, G7513) were added to each well. Cells were incubated with the antibody-coated beads for 18 hours at 37° C. After the 18 hour incubation period, cells were washed with PBS and fixed using 4% PFA. The proportion of cells containing fluorescently-labelled beads was measured using flow cytometry (BD, Fortessa X20).

Data were analysed with Flowjo (BD, Version 10), using a gating strategy to select the THP-1 cells. Normalised phagocytic scores were calculated by multiplying the percentage of bead-positive cells with the mean fluorescence intensity (MFI) of the beads within these cells and normalising against a QC sample set to 1. As multiple plates were run during an experiment, plates failed if any of the QC sample averages fell outside of the mean +/- 2 SD range of that particular QC across plates. In addition, samples were excluded from further analysis if the replicates showed a coefficient of variation (CV) over 25%. The data in the current paper are all derived from one experiment.

### Antibody Dependent Complement Deposition (ADCD)

SPHERO™ Carboxyl magnetic blue fluorescent beads (Spherotech, USA) were coupled with SARS-CoV-2 whole spike protein (Lake Pharma, USA, ref 46328) using a two-step Sulpho-NHS/EDC process detailed in Brown et al. Spike protein was included at saturation levels and coupling confirmed by the binding of IgG from a Covid-19 convalescent donor known to have high levels of anti-spike protein IgG.

Heat-inactivated test serum (2.5 µl, in duplicate) was added to 22.5 µl blocking buffer (PBS, 2% BSA, BB) and 5 µl taken for serial 5-fold dilutions to give final dilutions of 1:20, 1:100, 1:500, 1:2500. 20 µl of SARS-CoV-2 spike protein-coated magnetic beads (50 beads per µl) was added, and the mixture incubated at 25° C. for 30 min with shaking at 900 rpm. The beads were washed twice in 200 µl wash buffer (BB + 0.05% Tween-20) then resuspended in 50 µl BB containing 10% IgG- and IgM-depleted human plasma (prepared as per (Lesne et al. 2020)) and incubated at 37° C. for 15 min with shaking at 900 rpm. Beads were next washed twice with 200 µl wash buffer and resuspended in 100 µl FITC-conjugated rabbit anti-human C3c polyclonal antibody (Abcam, UK) and incubated at room temperature in the dark. After two more washes with 200 µl wash buffer, the samples were resuspended in 60 µl Hank’s Balanced Salt Solution and analysed using a CytoFLEX S flow cytometer (Beckman Coulter, USA) and CytExpert software. For each sample, a minimum of 100 beads were collected. The median FITC fluorescence of a complement-only, no antibody control was subtracted from each test sample to give the antibody-dependent median FITC fluorescence. Interpolated end-point titres were calculated in Microsoft Excel to determine the dilution where the antibody-dependent median FITC crossed the negative test threshold (the fluorescence obtained using pre-2020 samples confirmed as negative by ELISA + 3 times the standard deviation).

### Mesoscale Discovery Multiplexed Immunoassay (MIA)

Antigen-specific responses to ChAdOx1 nCoV19 vaccination and/or natural SARS-CoV-2 infection were measured using a multiplexed immunoassay (MIA). The MIA was developed and performed by Meso Scale Discovery (MSD), Rockville, MD, and is described in Folegatti et al (Folegatti et al. 2020). Briefly, dried plates coated with SARS-CoV-2 Spike and RBD were blocked, washed and incubated with samples, reference standards, and controls. Internal quality controls and reference standard reagents were developed from pooled human serum. Following incubation and washing steps, detection antibody was added (MSD SULFO-TAG™ Anti-Human IgG Antibody), incubated and plates washed again. MSD GOLD™ Read Buffer B was added and plates read using a MESO® SECTOR S 600 Reader. Samples at the lower limit of quantitation were set to 2.58 for Spike and 2.60 for RBD, while samples at the upper limit were set to 320000 for Spike and 317073 for RBD.

### Public Health England Microneutralisation Assay (PHE MNA80)

Using a similar method to that described above for PHE MNA80 (and described in (Folegatti et al. 2020), the microneutralisation assay (MNA) measures microplaques using the ImmunoSpot® S6 Ultra-V Analyzer. Briefly, serum/virus mixtures were added to monolayers of virus-susceptible Vero/E6 cells for one hour before replacement of inoculum with overlay (1% w/v CMC in complete media). Following a 24-hour incubation, cells were fixed with formaldehye. Microplaques were detected using a SARS-CoV-2 antibody specific for the SARSCoV-2 RBD Spike protein and a rabbit HRP conjugate. Infected microplaques were detected using TrueBlueTM substrate. Resulting counts analysed in SoftMax Pro v7.0 software.

### Monogram Biosciences Pseudotype Neutralisation Assay (PseudoNA)

Neutralizing antibody (Nab) titres were determined using a lentivirus-based SARSCoV-2 pseudovirus particle expressing spike protein (Accession number: MN908947.3). The pseudotype neutralisation CoV nAb assay is described in (Folegatti et al. 2020) and based on previously described methodologies using HIV-1 pseudovirions (Petropoulos et al. 2000; Richman et al. 2003; Whitcomb et al. 2007).

Briefly, heat inactivated, diluted serum samples were incubated with SARS-CoV2 pseudotyped virus. Nab titres were determined by creating 9 serial three-fold dilutions of test samples. Irrelevant pseudotyped virus was used as a control. Following incubation of diluted sera and pseudovirus particle, HEK 293 ACE2-transfected cells were added, plates were incubated and luciferase expression measured. Nab titres are reported as the reciprocal of the serum dilution conferring 50% inhibition (ID50) of pseudovirus infection. %Inhibition = 100% -(((RLU(Vector+Sample+Diluent) - RLU(Background))/(RLU(Vector+Diluent) -RLU(Background))) x 100%). SARS CoV-2 nAb Assay Positive and Negative Control Sera are included on each 96-well assay plate.

### Example 18: Detailed Phenotyping of the Immune Response Induced By ChAdO×1 nCoV-19 Vaccine in a Phase ½ Clinical Trial.

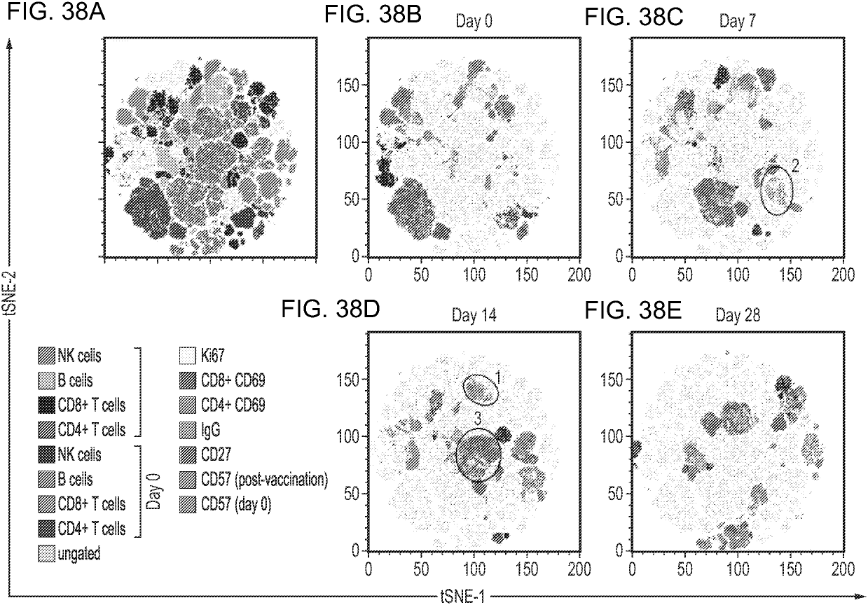

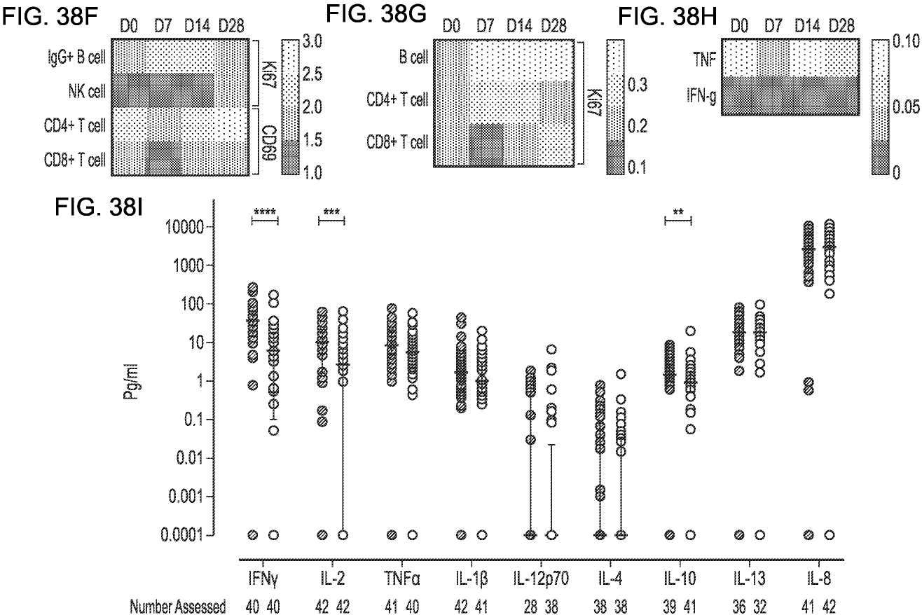

Example 18 relates to a phase I/II randomised controlled trial, it should be read in conjunction with the earlier examples, in particular Examples 11, 15, 16 and 17. FIGS. 38 to 43 show data relating to Example 18.

### Summary

Strong Th1- skewed T cell responses are expected to be optimal for driving protective humoral immune responses, infection-controlling cell-mediated immune responses and reducing the potential for disease enhancement. Here we describe in detail the immune responses in adults aged 18-55 years after ChAdOx1 nCOV-19 vaccination, demonstrating an induction of a Th1-biased response characterised by IFN-γ and TNFα cytokine secretion by CD4⁺ T cells and antibody production predominantly of the IgG1 and IgG3 subclasses, the latter of which correlated with neutralising activity. CD8⁺ T cells, of monofunctional, polyfunctional and cytotoxic phenotypes, were also induced. Taken together, these results suggest a favourable immune profile induced by ChAdOx1 nCoV-19 vaccine.

Here we provide a detailed description of the immune response following administration of one or two doses of ChAdOx1 nCoV-19. Importantly, we demonstrate with several methodologies (multiplex cytokine profiling, ICS analysis and antibody isotype profiling) that vaccination with ChAdOx1 nCoV-19 induces a predominantly Th1 response, assuaging concern around Th2 driven vaccine enhanced disease and indicating a potentially protective level of immune response induced by vaccination.

98 healthy adults aged 18-55 were vaccinated with 5 x 10¹⁰ viral particles of the candidate vaccine ChAdOx1 nCov-19 as part of a Phase I/II clinical trial. Blood samples were collected on the day of vaccination and 7, 14, 28 and 56 days post vaccination. Ten vaccinees received a second dose of the same vaccine four weeks later (day 28) and additional blood samples were collected at day 35 and 42 in these participants.

### Adaptive and Innate Immune Cells Are Activated and Proliferate After ChAdOx1 nCoV-19 Vaccination with Th1-Biased Cytokine Secretion

An unbiased approach was applied to measure gross phenotypic and cellular changes induced post-vaccination on days 7, 14, 28 post vaccination with ChAdOx1 nCoV-19 FIGS. 38A-38E).

Flow cytometric analysis of discrete immune populations was performed on unstimulated frozen PBMCs. Combined tSNE analysis of 26 ChAdOx1 nCoV-19 vaccinated volunteers formed discrete populations of T cells, NK cells, and B cells on day 0 and days 7, 14 and 28, indicating phenotypic changes in these cell populations post-vaccination. Within these clusters, distinct populations of proliferating (Ki-67⁺) or activated (CD69⁺) cells were identified FIGS. 38B-38E). Activated cell populations noticeably clustered within IgG⁺ B cell populations, CD4⁺ T cell populations, and NK cell populations.

B cells, especially the IgG+ B cell population, up-regulated the cellular proliferation marker Ki-67 at all post-vaccination time points FIGS. 38F and 38G). Within the total B cell population, the distinct shift towards an activated phenotype peaked on day 7 to day 28, and for the IgG⁺ B cell population on days 7-14 FIGS. 38F and 38G).

CD4⁺ T cells had increased expression of the activation marker CD69 on days 7 to 28 post-vaccination and a trend towards increased Ki-67 expression at days 7 and 14 post-vaccination FIGS. 38F and 38G). CD8⁺ T cells expressed a similar pattern of Ki-67 and CD69 expression between days 7 and 28 post-vaccination FIGS. 38F and 38G). There was no increase in the expression of the terminal differentiation markers CD57 and KLRG1 in post-vaccination CD8⁺ T cells, which would indicate a reduction in post-vaccination cytotoxic capacity³². After peptide stimulation, an increase in TNFα and IFNγ production by CD4⁺ T cells was also observed at day 14 FIG. 38H). These activation markers are typical of a Th1 driven response. A similar pattern of Ki-67 and CD69 expression between days 7 and 28 post-vaccination FIGS. 38F and 38G). There was no increase in the expression of the terminal differentiation markers CD57 and KLRG1 in post-vaccination CD8⁺ T cells which, if found, would indicate a reduction in post-vaccination cytotoxic capacity. After peptide stimulation, an increase in TNFα and IFNγ production by CD4⁺ T cells was also observed at day 14 FIG. 38H). These activation markers are typical of a Th1 driven response.

NK cells can elicit a cytotoxic response to viral infections and in response to vaccination. We demonstrate here that following vaccination with ChAdOx1 nCoV-19 the percentage of combined CD56⁺CD16⁺ and CD56⁺⁺CD16⁻NK cells increased slightly at day 7 and 14 post-vaccination and that the expression of Ki-67 by NK cells increased steadily to a peak at day 28 FIG. 38F). There was no significant change in the expression of CD57, indicating terminal differentiation, or the activating receptor NKG2C.

A multiplex cytokine analysis was performed on day 7 post-vaccination following antigen specific stimulation of PBMC. Of the nine cytokines analysed, five (IL1β, IL12, IL4, IL13 and IL8) showed no difference in expression levels following stimulation. IFNγ and IL2 secretion were significantly increased in ChAdOx1 vaccinees compared with controls (p<0.0001 and p=0.0003 respectively, Mann-Whitney test) with a modest increase in TNFα (p=0.09, Mann-Whitney test. IL4 and IL13 levels were not increased in ChAdOx1 vaccinees following stimulation of PBMC (p>0.05 for both), but a modest increase in IL10 was measured (p=0.005). The magnitude of cytokine secretion measured in ChAdOx1 vaccinees was greater for IFNγ (median 36.4 pg/ml, interquartile range [IQR] 15-67) and IL2 (median 10.7 pg/ml, IQR 1.7-22), than for IL10 (median 1.4 pg/ml, IQR 0.9-2.6) indicating a strong bias towards secretion of Th1 cytokines FIG. 38I).

### Immune Responses to ChAdOx1 nCoV-19 Do Not Differ by Sex

Female COVID-19 patients show more robust T cell activation than male patients and poor T cell response negatively correlated with patients’ age, which was associated with worse disease outcome in male patients. It has also been previously demonstrated that females mount stronger antiviral immune responses. Robust immunity induced by ChAdOx1 nCOV-19 against the SARS-CoV-2 spike antigen, measured by ex vivo IFNγ ELISpot and total IgG ELISA was previously reported. We analysed these two main immunological outcome measures by sex and age. We found no sex difference in vaccine response after a single dose of vaccine at any of the time points measured (p>0.05, Mann-Whitney test). We detected no association between age and magnitude of immune response for either outcome measure (p=0.7, Spearman’s correlation) in this population aged between 18 and 55 years.

### ChAdOx1 nCoV-19 Vaccination Induces SARS-CoV-2-specific IgM and IgA as Well as IgG After Prime or Prime-Boost Vaccination Regimens

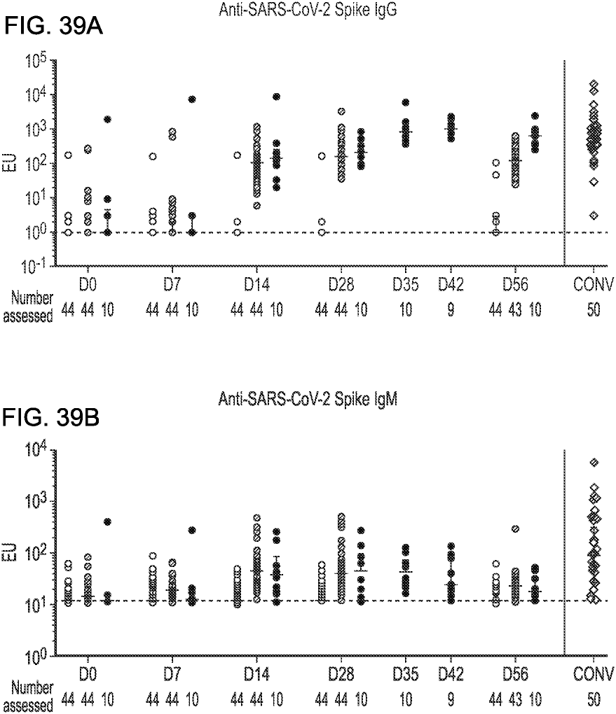

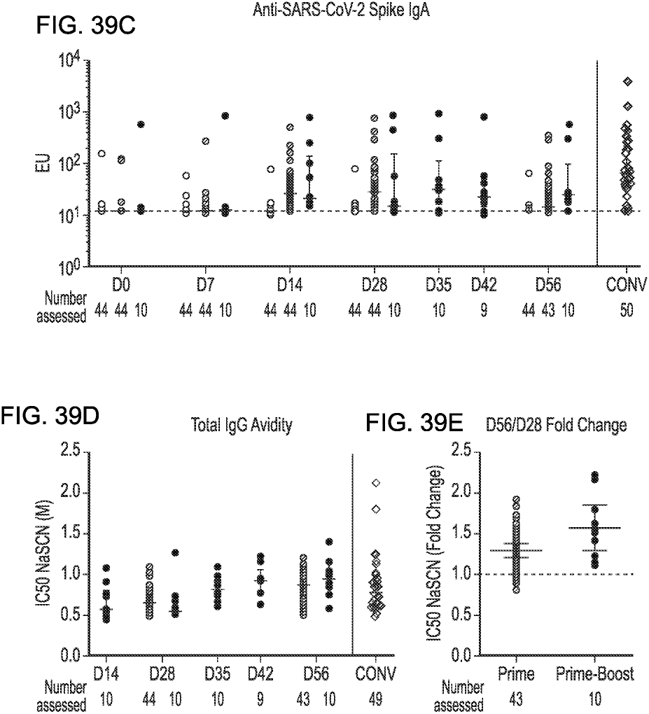

A comprehensive isotype (IgM, IgA and IgE) analysis of anti-SARS-CoV-2 spike antibodies was performed. We included IgG analysis as previously reported with the inclusion of additional data points. SARS-CoV-2 spike-specific antibody responses were quantified by standardised ELISA for trial participants receiving either MenACWY or ChAdOx1 nCoV-19 vaccination and are shown in FIG. 39.

Total IgG responses against spike protein were detectable at day 14, peaked at day 28 and were maintained at day 56 (FIG. 39A; table 1). Vaccination with ChAdOx1 nCoV-19 also generated increased levels of SARS-CoV-2 spike-specific IgM and IgA with peak responses at day 14 or day 28, respectively (FIGS. 39B and 39C; table 1).

As previously described, total IgG responses increased following a second dose of ChAdOx1 nCoV-19 administered 4 weeks after the first dose (FIG. 39A; table 1). In this subset of vaccinees, a peak response was detected at day 42 following initial vaccination. IgM and IgA responses were maintained at similar levels after prime and booster vaccination (FIGS. 39B and 39C; table 1), without significant difference at day 56 in the prime-boost group when compared with the prime group only (p=0.767 and p=0.092, respectively, Mann-Whitney test). Detailed profiling of immunoglobulin isotypes was performed on plasma samples from convalescent COVID-19 patients. While SARS-CoV-2 spike-specific IgG responses in these individuals were at similar levels to ChAdOx-1 nCoV-19 vaccinees after the prime-boost regimen, IgM and IgA responses induced by vaccination were in general lower than those induced after natural infection

(FIGS. 39A, 39B and 39C). Low SARS-CoV-2 spike-specific IgE responses were detected following vaccination with ChAdOx1 nCoV-19 which was similar to that measured after natural exposure to SARS-CoV-2.

The avidity of SARS-CoV-2 spike-specific IgG from volunteers who had a quantifiable IgG response at day 28, was assessed with a sodium thiocyanate displacement ELISA. IgG avidity increased between day 28 (median 0.66, IQR 0.60-0.76; n=45) and day 56 in the prime only group (median 0.88, IQR 0.74-0.94; n=44) and in the prime-boost group (day 28 median 0.54, IQR 0.52-0.68; n=10; day 56 median 0.94, IQR 0.83-1.15; n=10) (FIG. 39D). Although the values for avidity were comparable on day 56 between both vaccination regimens, there was a higher fold change from baseline between values on day 28 and day 56 in the prime-boost group (mean 1.58, SD 0.39; n=10) when compared with the prime only group (mean 1.30, SD 0.27; n=44) (p=0.0093, unpaired t test) (FIG. 39E). The IgG avidity generated by one dose of ChAdOx1 nCoV-19 by day 56, or a booster dose of ChAdOx1 nCoV-19 by day 35, exceeded IgG avidity measured in samples from convalescent COVID-19 patients (median 0.77, IQR 0.62-0.92; n=49).

### Subclass Analysis After Prime or Prime-boost Vaccination with ChAdOx1 nCoV-19

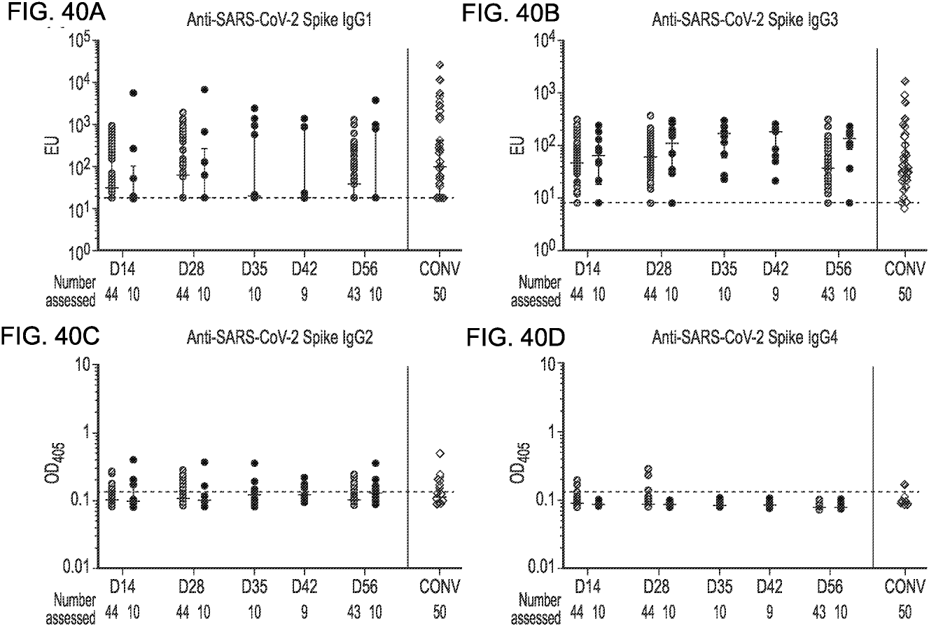

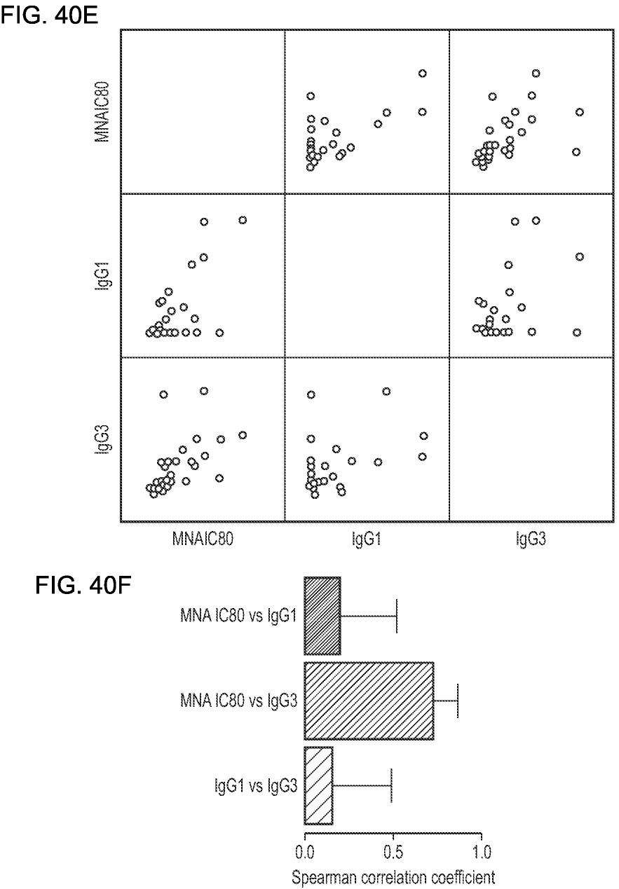

Samples from participants vaccinated with ChAdOx1 nCoV-19 with detectable levels of total IgG at day 14 post vaccination, were assayed for anti-spike IgG subclasses (FIG. 40). SARS-CoV-2 spike-specific IgG1 and IgG3 responses were readily detectable at day 14, increased by day 28 and returned to a similar level to that measured on day 14 by day 56 (FIGS. 40A and 40B; table 1). While IgG3 responses were quantifiable in the vast majority of vaccinees (day 14 39/44; day 28 42/44 and day 56 39/44), IgG1 responses were quantifiable in approximately half of the vaccinees (day 14 24/44; day 28 23/44 and day 56 22/44). Following booster vaccination, the median IgG1 response of volunteers did not increase above baseline, with similar levels at day 56 compared with the prime-only group (p=0.8; Mann-Whitney test). IgG3 responses increased following booster vaccination and were significantly higher at D56 compared with the prime-only group (p=0.003, Mann-Whitney test). Median levels of IgG2 and IgG4 were low across all groups and time points (FIGS. 40C and 40D). Similar mixed IgG3/IgG1 profile with low levels of IgG2 and IgG4 were measured in convalescent plasma samples. In agreement with previously reported data, SARS-CoV-2 spike-specific IgG1 responses were below the limit of quantitation in some convalescent plasma samples (FIGS. 40A, 40B, 40C and 40D). Finally, we analysed the correlation between IgG3 levels and neutralisation capacity for the same vaccinees, as reported previously (microneutralisation assay (MNA₈₀)) demonstrating a correlation between IgG3 levels and MNA₈₀ (Spearman r = 0.73; 95% CI 0.51-0.86; p < 0.001) (FIG. 40E).

### ChAdOx1 nCoV-19 Induces a Broad T Cell Response to the S1 and S2 Subunits of the SARS-CoV-2 Spike Antigen

T cell responses were measured by IFNγ ELISpot before and after vaccination with ChAdOx1 nCoV-19, peaking at day 14. Responses were assayed against 13 pools of overlapping peptides (table S1) spanning the length of the vaccine antigen insert, which includes the S1 and S2 subunits, and an exogenous human tissue plasminogen activator (tPA) leader signal sequence peptide previously shown to enhance immunogenicity of a MERS-CoV vaccine candidates in mice. All peptide pools except the tPA signal peptide elicited a positive response (defined by the mean of the negative control plus 4 SDs) in at least 25% of the participants, indicating recognition of multiple epitopes in both spike subunits. The most frequently recognized pools were 4 and 2 which span amino acid 311 to 430 and 101 to 200 of the S1 domain and generated a positive response by IFNγ ELISpot in 88% and 86% of participants, respectively.

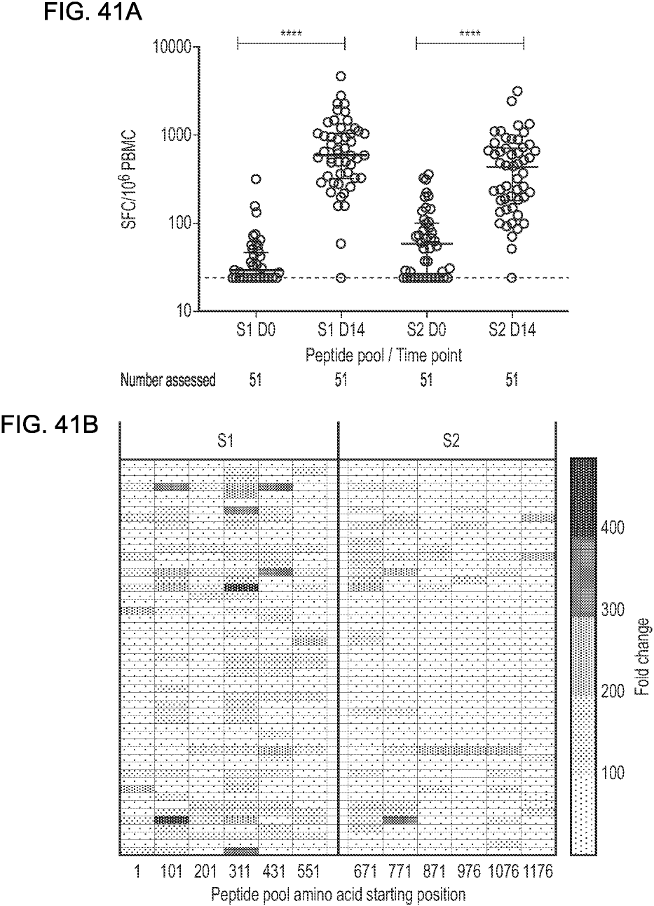

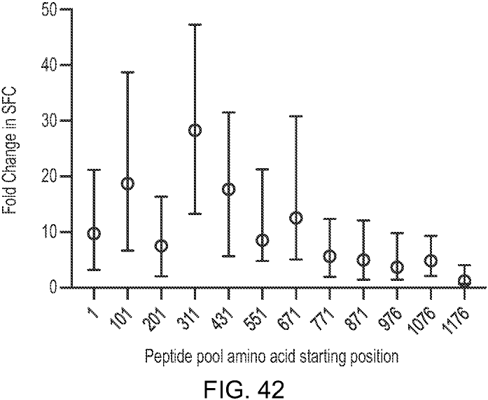

As positive responses to some peptide pools were detectable in a small proportion of participants prior to vaccination (FIG. 41A), responses to the individual pools at D14 were plotted as fold-change from D0 (FIG. 41B and FIG. 42). The greatest increases were detected against pools 4 and 2, both corresponding to the S1 subunit. These pools elicited a median response of 165 SFC/10⁶ PBMCs and 124 SFC/10⁶ PBMCS respectively at day 14, equating to a median of a 28 and 19-fold change from baseline. T cell responses were detected across both the S1 and S2 subunits, but T cell responses to the tPA signal sequence in the vaccine construct were not detected.

### Vaccination Induces a Th1-biased CD4⁺ T Cell Response and a Cytotoxic CD8⁺ T Cell Response Against SARS-CoV-2 Apike Ppeptides

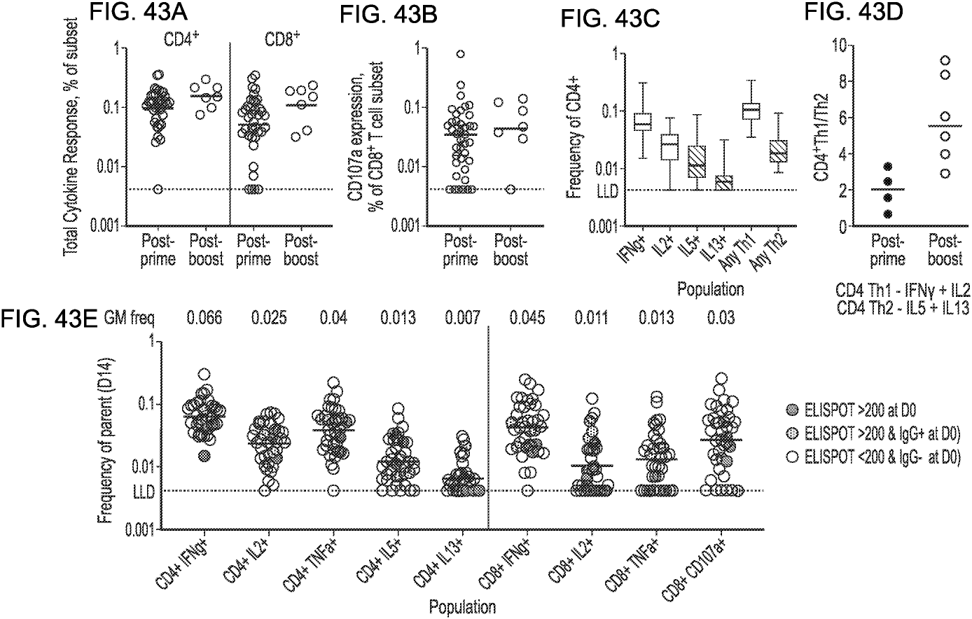

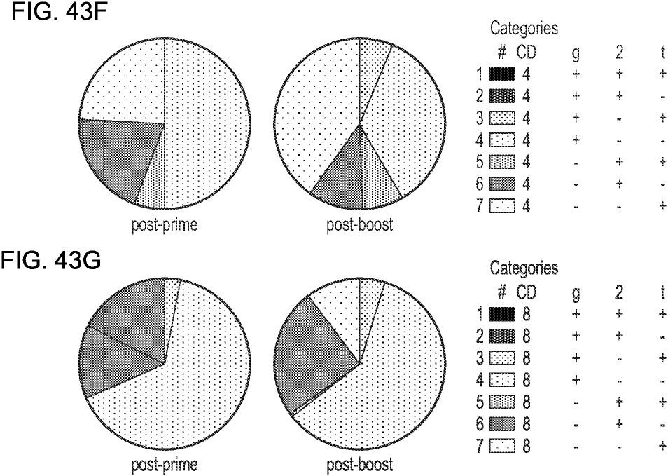

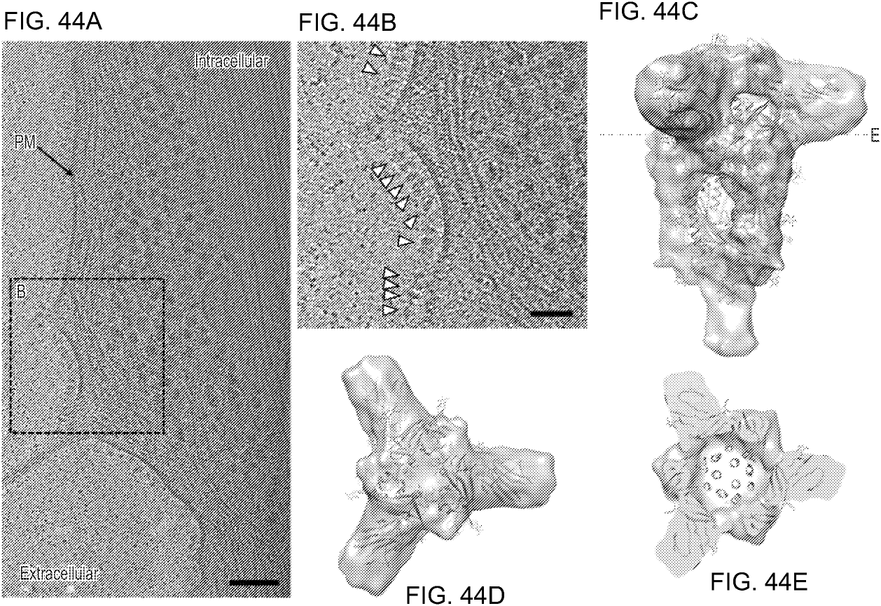

Flow cytometry with intracellular cytokine staining of PBMC stimulated with peptides spanning the S1 and S2 subunits of SARS-CoV-2 spike protein demonstrated antigen-specific cytokine secretion from both CD4⁺ (GM 0.1%, 95% CI 0.08-0.13) and CD8⁺ (0.05%, 95% CI 0.03-0.08) T cells 14 days after a single dose of ChAdOx1 nCoV-19. After a second dose, frequencies increased for both CD4⁺ (GM 0.16%, 95% CI 0.1-0.24) and CD8⁺ (0.11%, 95% CI 0.05-0.23) T cells, although increases were not statistically significant (FIG. 43A). CD8⁺ T cells expressed the degranulation marker CD107a indicating cytotoxic function (GM 0.03%, 95% CI 0.02-0.05), which again increased after boosting (GM 0.05%, 95% CI 0.015-0.14) (FIG. 44b). CD4⁺ responses were heavily biased towards secretion of Th1 cytokines (IFNγ and IL2) rather than Th2 (IL5 and IL13, FIG. 43C) and the ratio of cytokine secretion increased further towards a Th1 biased phenotype after boosting (FIG. 43D). The frequency of cytokine positive cells was generally higher in the CD4⁺ T cell population than the CD8⁺population and cytokine responses were detected at day 14 from participants with positive pre-vaccination T cell and antibody responses to SARS-CoV-2 (FIG. 43E). When combinations of cytokines were assessed, few multifunctional T cells were detected in either the CD4⁺ or CD8⁺ populations (FIGS. 43F and 43G). Responses were dominated by T cells expressing single cytokines, particularly monofunctional IFNγ⁺ CD8⁺ T cells and the multifunctionality of the response was not increased by homologous boosting with a second dose. Some dual positive CD4⁺ and CD8⁺ T cells were detectable, expressing a combination of IFNγ and TNFα.

The Figures for Example 18 are as follows:

### FIG. 38. Activation of Lymphocyte Populations Post ChAdO×1 nCoV-19 Vaccination

(A-E) tSNE analysis of PBMC lymphocyte populations from 26 ChAdOx1 nCoV-19 vaccine trial participants A) global clustering of immune cells across all samples B-E) tSNE population analysis at day 0 and days 7, 14, and 28 post-vaccination. Areas of Ki-67+ activity (yellow) cluster in IgG⁺ B cells (1), NK cells (2), and CD4⁺ T cells (3) post-vaccination. Analysis conducted on unstimulated cells.

(F-H) Heatmap analysis of activation markers expressed by immune cells at days 0, 7, 14 and 28 post- ChAdOx1 nCoV-19 vaccination. F) Expression of Ki-67 by IgG⁺B cells and NK cells (top two rows), and NK cells (top three rows). Expression of CD69 by CD4⁺ T cells and CD8⁺ T cells (bottom two rows). G) Expression of Ki-67 by B cells, CD4⁺ T cells and CD8⁺ T cells. H) Expression of TNFα and IFNγ by CD4⁺ T cells. Analysis of TNFα and IFNγ expression conducted on cells stimulated with spike glycoprotein peptide pools with unstimulated values subtracted. All other analysis conducted on unstimulated cells.

I. A multiplex cytokine analysis was performed on day 7 post-vaccination using supernatants following antigen specific stimulation of PBMC. ChAdOx1 nCov-19 group shown in blue and MenACWY group shown in red. Significant difference observed between IFNγ (P<0.0001), IL-2 (P=0.0003) and IL-10 (P=0.0051) Mann-Whitney. Lines are shown at the median with error bars showing IQR.

### FIG. 39. Immunoglobulin Isotype Responses Induced by ChAdO×1 nCoV-19 or MenACWY Vaccination

Volunteers in the prime group received a single ChAdOx1 nCoV-19 or MenACWY vaccination at day 0. Volunteers in the boost group received a ChAdOx1 nCoV-19 at day 0 and day 28. Data for convalescent plasma samples (CONV) from recovered SARS-CoV-2 patients are shown. CONV symbols are shaded by disease severity, indicating asymptomatic, mildly symptomatic, moderately symptomatic, severely symptomatic or symptomatic. The MenACWY group, the ChAdOx1 prime group, and the ChAdOx1 prime-boost group are shown.

(A-C) SARS-CoV-2 spike trimer-specific antibody responses were quantified by standardised ELISA and expressed as ELISA units (EU). Dotted lines are shown at the limit of quantification of each assay, details of which are given in the materials and methods. Lines are shown at the median with error bars showing the IQR.

(D) Avidity of SARS-CoV-2 spike trimer-specific IgG antibody responses was measured using a NaSCN chemical displacement ELISA and expressed as an IC50. Lines are shown at the median with error bars showing the IQR.

(E) Fold change of avidity between day 56 and day 28. Lines are shown at the mean, with error bars showing the 95% CI. The dotted line represents no change in avidity. A value greater than one indicates an increase in avidity from day 28 to day 56.

### Table 18_1. Summary Antibody Data (See Below)

Anti-SARS-CoV-2 Spike Immunoglobulins levels in serum of volunteers (n) after prime or prime-boost vaccination with ChAdOx1-nCoV-19, presented as median with interquartile range (IQR).

TABLE 18_1

Anti-SARS-COV-2Spike Immunoglobins levels in serum
ChAdx1 nCoV-19 Prime

| | IgG | | | IgM | | | IgA | | | IgG1 | | | IgG3 | | |
|---|---|---|---|---|---|---|---|---|---|---|---|---|---|---|---|
| | n | Median | IQR | n | Median | IQR^{∗} | n | Median | IQR | n | Median | IQR | n | Median | IQR |
| (EU) | | | (25%-75%) | | | (25%-75%) | | | (25%-75%) | | | (25%-27%) | | | (25%-75%) |
| D0 | 44 | 1.00 | 1.00-1.00 | 44 | 14.37 | 12.00-22.35 | 44 | 12.00 | 12.00-12.00 | | | | | | |
| D7 | 44 | 1.00 | 1.00-2.00 | 44 | 14.17 | 12.00-22.35 | 44 | 12.00 | 12.00-12.00 | | | | | | |
| D14 | 44 | 103.00 | 38.50-188.80 | 44 | 44.80 | 25.71-66.05 | 44 | 26.09 | 17.21-57.64 | 44 | 30.90 | 18.00-224.60 | 44 | 46.62 | 20.16-89.94 |
| D28 | 44 | 165.00 | 105.00-336.50 | 44 | 39.90 | 26.85-73.64 | 44 | 28.26 | 14.08-47.28 | 44 | 62.59 | 18.00-504.70 | 44 | 59.78 | 33.71-123.20 |
| D56 | 43 | 121.00 | 73.00-217.00 | 43 | 22.85 | 13.26-32.38 | 43 | 14.23 | 12.00-26.57 | 43 | 37.98 | 18.00-246.30 | 43 | 36.62 | 19.13-64.50 |

ChAdOx1 nCov-19 Prime-Boost

| | IgG | | | IgM | | | IgA | | | IgG1 | | | IgG3 | | |
|---|---|---|---|---|---|---|---|---|---|---|---|---|---|---|---|
| | n | Median | IQR | n | Median | IQR | n | Median | IQR | n | Median | IQR | n | Median | IQR |
| (EU) | | | (25%-75%) | | | (25%-75%) | | | (25%-75%) | | | (25%-75%) | | | (25%-75%) |
| D0 | 10 | 1.00 | 1.00-4.50 | 10 | 12.00 | 12.00-15.08 | 10 | 12.00 | 12.00-12.52 | | | | | | |
| D7 | 10 | 1.00 | 1.00-3.00 | 10 | 12.48 | 12.00-18.09 | 10 | 12.00 | 12.00-13.32 | | | | | | |
| D14 | 10 | 137.00 | 29.75-256.30 | 10 | 37.97 | 17.64-86.71 | 10 | 20.88 | 16.06-138.1 | 10 | 18.00 | 18.00-104.50 | 10 | 64.09 | 17.93-143.30 |
| D28 | 10 | 211.00 | 135.30-376.00 | 10 | 45.57 | 18.52-97.15 | 10 | 14.59 | 12.57-152.8 | 10 | 18.00 | 18.00-262.00 | 10 | 110.80 | 32.87-219.80 |
| D35 | 10 | 821.00 | 538.30-1419.00 | 10 | 43.50 | 24.13-74.48 | 10 | 31.70 | 16.86-112.30 | 10 | 19.66 | 18.00-1040.00 | 10 | 167.50 | 58.04-246.60 |
| D42 | 9 | 997.00 | 598.00-1338.00 | 9 | 24.24 | 16.98-86.69 | 9 | 22.40 | 14.64-49.12 | 9 | 18.00 | 18.00-876.90 | 9 | 186.00 | 58.04-241.00 |
| D56 | 10 | 639.50 | 349.30-860.80 | 10 | 17.86 | 14.47-36.16 | 10 | 24.52 | 16.35-95.69 | 10 | 18.00 | 18.00-860.00 | 10 | 133.50 | 83.21-190.60 |

*IQR-interquartile range

### FIG. 40. IgG Subclass Responses Induced by a Single Dose or Prime-Boost Regimen of ChAdOx1 nCoV-19

Volunteers in the single dose (prime) group (red) received a ChAdOx1 nCoV-19 vaccination at day 0. Volunteers in the prime-boost group (purple) received a ChAdOx1 nCoV-19 vaccination at day 0 and day 28.

(A-B) SARS-CoV-2 spike trimer-specific antibody responses were quantified by standardised ELISA and expressed as ELISA units (EU). Dotted lines are shown at the limit of quantification of each assay, details of which are given in the materials and methods.

(C-D) SARS-CoV-2 spike trimer-specific antibody responses were measured by indirect ELISA and expressed as OD₄₀₅. Volunteers that were seropositive for total IgG at day 0 or seronegative at day 28 were excluded from the analysis regardless of vaccination status. Data shows only ChAdOx1 nCoV-19 vaccinated volunteers. Dotted lines are shown at the mean cut-off for each assay. Cut-offs were calculated for each plate according to materials and methods.

(A-D) Lines are shown at the median with error bars showing the IQR. Responses for convalescent plasma samples (CONV) from recovered SARS-CoV-2 patients are shown. CONV symbols are shaded by disease severity, indicating asymptomatic, mildly symptomatic, moderately symptomatic, severely symptomatic or symptomatic.

(E) Relationship between Microneutralisation Assay (MNA IC80) data and trimer-specific IgG3 and IgG1 levels. Correlation matrix scatterplot (E) and Spearman correlation coefficient (mean with 95% confidence interval) analysed at day 28.

### FIG. 41: IFNγ ELISPOT Responses to Pools of 15mer Peptides Covering the ChAdOx1-nCOV19 Vaccine

(A) Total response to S1 and S2 (sum of 6 peptide pools each) at D0 and D14 post-vaccination. Bars show median SFC per million PBMC with error bars representing the IQR. The dashed line represents the lower limit of detection of the assay (48 SFC). (B) Heat map of fold-change in SFC to each peptide pool for every participant from baseline (D0) to D14 post- vaccination. Only data from ChAdOx1 vaccinees is shown.

FIG. 42: Fold-change in SFC to each peptide pool for every ChAdOx1 vaccinated participant from baseline (D0) to D14 postvaccination. Circles show median fold change with bars representing IQR.

### FIG. 43. T Cell Responses to SARS-CoV-2 Spike Peptides Measured by Flow Cytometry with Intracellular Sytokine Staining

(A) Frequency of CD4+ or CD8+ T cells expressing IFNγ, IL2 or TNFα at 2 weeks after prime or boost dose (n=7). Circles represent individual participants and lines are geometric means. (B) Frequency of CD8+ T cells expressing CD107a+ at 2 weeks after prime or boost dose. (C) Frequency of Th1 and Th2 cytokine secretion by CD4+ T cells at 2 weeks after one dose (n=36). (D) Ratio of Th1 to Th2 cytokine secretion at 2 weeks after first or second dose. (E) Frequency of CD4+ or CD8+ T cells expressing relevant individual cytokines. Dotted lines show lower limit of detection (LLD). (F) and (G) Expression of cytokine combinations from CD4+ (F) and CD8+ (G) T cells. In all panels, n=39 participants post-prime and n=7 post-boost. Only data from ChAdOx1 vaccinees is shown.

### Discussion

We have described here, in detail, the profile of cytokine expression from both CD4⁺ and CD8⁺ T cells and the IgG subclass composition of the antibody response to the ChAdOx1 nCoV-19 vaccine. Robust B cell activation and proliferation is observed after vaccination with ChAdOx1 nCoV-19 and anti-IgA and IgG antibodies to the SARS-CoV-2 spike protein are readily detected in the sera from ChAdOx1 nCoV-19 vaccinated volunteers. The temporal production of antibody subclasses was similar to that reported from COVID-19 patients and the latter resembles a typical viral infection with early responses from short-lived extrafollicular B cells followed by germinal centre reactions and long-lived memory B cell responses. Anti-spike IgG responses at the peak of the response post-vaccination shows a polarized IgG1 response which is consistent with naturally acquired antibodies against SARS CoV-2. The increase in IgG3 after boost suggests this subclass may underpin the functional increase in neutralising antibody titres. Produced early after viral infections, IgG3 coordinates multiple antibody effector functions, which are crucial for rapid clearance and may contribute to recovery after SARS-CoV-2 infection. A mixed IgG1 and IgG3 response, with low levels of IgG2 in a subset of volunteers and little detectable IgG4 is in agreement with previously published reports describing the induction of Th1-type human IgG subclasses (IgG1 and IgG3) following adenoviral priming.

Pathology consistent with vaccine enhanced disease and in one case antibody dependent enhancement of infection (ADE) was demonstrated in animal models for some SARS-CoV-1 vaccine candidates, and there is concern that a similar pathological response may be induced in humans after vaccination against SARS-CoV-2. Vaccine enhanced disease results in an increase in disease severity when vaccinated subjects are subsequently exposed to or challenged with natural virus. A number of preclinical studies of candidate SARS-CoV-1 vaccines in mouse, ferret and non-human primate (NHP) model reported disease enhancement when vaccinated animals were challenged with live virus. The phenomenon is reminiscent of the disease observed with early vaccine development against respiratory syncytial virus (RSV), wherein pathology was associated with a high ratio of non-neutralising antibodies to neutralising antibodies, a role for neutrophils, eosinophils and predominantly a Th2 biased response. Vaccine-enhanced disease differs from ADE and original antigenic sin (OAS) or imprinting. The former phenomenon can occur following flavivirus infection or vaccination and the latter is readily observed following recurrent influenza infection. Vaccination with an MVA-vectored vaccine expressing the SARS-CoV-1 spike protein was associated with ADE in the ferret model of infection. The neutralizing antibody responses induced by vaccination were low and it is has been demonstrated that antibodies capable of neutralisation at high titre can result in ADE at low titre. In contrast neutralising antibody titres induced after the first dose of ChAdOx1 nCoV-19 are high, are further increased following a second dose and resulted in reduced disease in vaccinated and challenged NHPs. The relative abilities of adenoviral and MVA-vectored vaccines to prime neutralising antibodies against the encoded antigen has been demonstrated clearly by Anywaine et al, suggesting that MVA-vectored vaccines against SARS-CoV-2 may be best deployed as the second part of a heterologous prime-boost regimen. It will also be important to investigate alternate prime boost regimens using technologies what are rapidly and sustainably scalable including heterologous adenoviral prime-boost regimens.

ChAdOx1 nCoV-19 induces a broad and robust T cell response to both subunits of the S antigen and the functionality of T cell response observed here is similar in phenotype of those observed with other replication deficient adenoviral vectors with responses dominated by individual T cells secreting single, rather than multiple, cytokines. Whether vaccine-induced monofunctional or polyfunctional T cells are of greater protective value appears to vary by disease and is unclear for SARS-CoV-2 infection and COVID-19. Analysis of cytokine secretion following peptide stimulation of PBMC demonstrated that IFNγ and IL2 secretion were increased in ChAdOx1 vaccinees compared with controls and, importantly from a safety perspective, IL4 and IL13 levels were not increased. Phenotyping by flow cytometric analysis demonstrated CD4⁺ responses were biased towards secretion of Th1 cytokines (IFNγ, IL2 and TNFα) rather than Th2 (IL5 and IL13). The bias we observed towards Th1-type cytokine secretion by vaccine-induced T cells has also been reported with other candidate vaccines in clinical trials utilising a variety of vaccine technologies. Importantly we demonstrate with several methodologies (multiplex cytokine profiling, ICS analysis and antibody isotype profiling) that vaccination with ChAdOx1 nCoV-19 induces a predominantly Th1 response and the application of these subtly different approaches allows nuances within T cell responses to be highlighted.

An important aspect in the epidemiology of COVID-19 disease is the marked difference in the mortality rates from disease between males and females, despite similar case rates. We therefore disaggregated the data for the key immunological outcome measures in these studies and have demonstrated no difference in the magnitude of either cellular or total IgG antibody responses between male and female participants or with increasing age. It will be important to continue to analyse disaggregated data across different ethnicities, different age groups, and also for the assessment of immune response durability, with consideration given to comorbidities which may further influence vaccine-induced immunity.

Although there are no defined immune correlates of protection against COVID-19, it is generally accepted, extrapolating from animal studies, that high-titre neutralising antibodies, with a robust cytotoxic CD8⁺ T cell response and Th1 biased CD4⁺ effector response will be optimal for protective immunity following SARS-CoV-2 exposure. The addition of a cell-mediated component to an antibody-inducing vaccine is attractive when the immune correlates for a disease are unknown and the induction of a cytotoxic CD8⁺ T cell response as described here is a useful adjunct to the induction of neutralising antibodies. Determining the precise threshold and phenotype of immune responses associated with protection will be important for bridging between populations and vaccines should any of these vaccines demonstrate useful efficacy against infection or disease. The detailed immunophenotyping of vaccine-induced immunity described here demonstrates both strong humoral and cellular immune responses after a single dose, characterised by a Th1-dominated response.

### Study Procedures and Sample Processing

Full details on the conduct of the Phase I/II randomised controlled trial of ChAdOx1 nCoV-19 (AZD1222), including the trial protocol, were discussed herein. This study was registered at ISRCTN [15281137] and ClinicalTrials.gov [NCT04324606].

At timepoints for immunological analyses, blood samples were taken in both plain and heparinised collection tubes. Samples were processed within 4 hours of the blood draw. Plain tubes were processed for the collection of blood serum. Tubes were centrifuged at 1800 rpm for 5 minutes and the serum harvested for storage at -80° C. until required. Heparinised tubes were processed for the collection of peripheral blood mononuclear cells (PBMCs) and blood plasma by density gradient centrifugation. Blood was decanted into Leucosep tubes (Greiner Bio-One) containing Lymphoprep (STEMCELL Technologies) and centrifuged at 1000 x g for 13 minutes with the brake off. A fraction of blood plasma was collected and stored at -80° C., whilst the remaining sample was decanted into a fresh falcon tube and topped up with R0 media (RPMI-1640 cell culture media containing 1% penicillin/streptomycin and 2 mM L-glutamine (all Sigma-Aldrich). Samples were centrifuged again at 1800 rpm for 5 mins, the supernatant poured off and the cell pellet resuspended once more in R0 for washing. After centrifugation, the cell pellet was resuspended in 10 ml of R10 media (RPMI-1640 containing 1% penicillin/streptomycin, 2 mM L-glutamine and 10% foetal calf serum (FCS, Labtech Intl.) for counting.

Cells were counted using a CasyCounter (OMNI Life Science) for use in fresh assays or for cryopreservation. The assays performed on fresh cells were ELISPOT and intracellular cytokine staining only (described below). All remaining cells were frozen at a concentration of 8-12 x 10⁶ PBMCs per ml. After centrifugation (1800 rpm, 5 mins) cells were resuspended in cold FCS at half the total freeze-down volume. Cells were placed in a refrigerator (2-8° C.) for 20 mins before an equal volume of cold FCS containing 20% dimethylsulphoxide was added. 1 ml aliquots were prepared and quickly transferred to CoolCells (Corning) for freezing at -80° C. overnight. Tubes were then transferred to a -150° C. ultra-low temperature freezer until required.

Convalescent plasma samples from adults (≥18 years) with PCR-positive SARS-CoV-2 infection were obtained from patients admitted to hospital or from surveillance on health-care workers, all of whom were enrolled in clinical studies (Gastrointestinal Illness in Oxford: COVID substudy [Sheffield Research Ethics Committee reference: 16/YH/0247], ISARIC/WHO Clinical Characterisation Protocol for Severe Emerging Infections [Oxford Research Ethics Committee C reference 13/SC/0149], and Sepsis Immunomics project [Oxford Research Ethics Committee C, reference 19/SC/0296]) Both asymptomatic and symptomatic participants were tested for each assay. Different samples were analysed across the assays, dependent on sample availability, laboratory capacity, and assay-specific requirements. Where multiple longitudinal samples were available for the same participant, only one timepoint is included in the analyses in this article and the earliest timepoint (at least 20 days post initial symptoms) was selected.

### Peptides and Stimulations

Peptides spanning the full length of the SARS-CoV-2 spike protein sequence were synthesised for use in antigen-specific T cell assays (Proimmune Ltd.). A total of 253 peptides were synthesised as 15-mers overlapping by 10 amino acids.

Peptides were also synthesised for the N-terminal tissue plasminogen activator (tPA) leader sequence which is included to increase expression of the vaccine antigen from the adenoviral vector. Peptide 1 started at amino acid position 1 and had the sequence MFVFLVLLPLVSSQC (SEQ ID NO: 19); Peptide 2 started at amino acid position 6 and had the sequence VLLPLVSSQCVNLTT (SEQ ID NO: 20); Peptide 3 started at amino acid position 11 and had the sequence VSSQCVNLTTRTQLP (SEQ ID NO: 21) and so on. Peptides 254 to 258 were overlapping 15mers in the same manner, but having the sequence from tPA. Briefly, for the Cytek™ Aurora flow cytometry assay, MSD Th1/Th2 cytokine profiling assay and intracellular cytokine staining, two separate peptide pools were made spanning the S1 (134 peptides) and S2 (119 peptides) subunits of the SARS-CoV-2 spike protein. For the ELISPOT assay, 12 pools of between 18-24 peptides were made consisting of 6 pools each for the S1 and S2 subunits. A separate tPA leader sequence pool (5 peptides) was included in this assay. Pool1: S1 - peptides 1 to 20; pool 2: (S1) - peptides 21-40; pool 3 - peptides 41 to 62; pool 4 - peptides 63 to 86; pool 5 - peptides 87 to 110; pool 6 - peptides 111 to 134; pool 7: S2 - peptides 135 to 154; Pool 8: (S2) - peptides 155 to 174; Pool 9 - peptides 175 to 195; Pool 10 - peptides 196 to 215; Pool 11 - peptides 216 to 235; pool 12 - peptides 236 to 253; tpa pool (5 peptides - peptides 254-258).

### Flow Cytometry Conducted on Cytek Aurora Spectral Analyser

Flow cytometry was performed from frozen aliquots of peripheral blood mononuclear cells (PBMCs) of 30 donors from days 0, 7, 14 and 28 after vaccination with ChAdOx1 nCoV19 (n=26). Cells were defrosted in media containing >5 U/mL benzonase and re-suspended in complete RPMI media supplemented with 10% FCS, L-glutamine and Penicillin/streptomycin at a concentration of 2x10⁷ cells/mL. 2x10⁶ PBMCs per well were plated in a 96-well plate and stimulated with synthetic peptides spanning the SARS-CoV-2 spike protein split into two separate pools for the S1 and S2 subunits (table S2) at a final concentration of 2 µg/mL, or media as a control. One well per donor was stimulated with Phorbol 12-myristate 13-acetate and ionomycin (Cell Activation Cocktail, BioLegend) as a positive control. PBMCs were co-stimulated in the presence of anti-human CD28, CD49d (1 µg/mL, Life Technologies Ltd), and CD107a-BV785 (BioLegend) for two hours at 37° C. with 5% CO₂, and then incubated for a further 16 hours after the addition of 1 µg/mL Brefeldin A and Monensin to each well (BioLegend).

PBMCs were washed in FACS buffer (Phosphate Buffered Saline with 0.5% bovine serum albumin and 1% EDTA) and stained with a cocktail of surface antibodies including anti-human Live/Dead-Zombie UV, CD4-AF700, CD19-Spark NIR 685, CD56-APC, CCR7-PerCP/Cy5.5, PD1-PE/Dazzle 594, CD57-PE/Cy7(BioLegend) CD8-AF405, CD45RA-SuperBright 702, CD27-PerCP eF710, CD20-AF532 (ThermoFisher Scientific) CD16-BUV495, CD3-BUV661, CD138-BUV805, NKG2A-BV480, IgM-BB515 (BD Biosciences), NKG2C-PE, KLRG1-VioBlue (Miltenyi) in FACS buffer with 10% Brilliant Stain buffer Plus (BD Biosciences). PBMCs were incubated at 4° C. in the dark for 30 minutes, then washed twice in FACS buffer. PBMCs were then incubated in CytoFix/CytoPerm solution (BD Biosciences) at 4° C. in the dark for 30 minutes, then washed twice in Perm/Wash buffer, and then stained with a cocktail of intracellular antibodies including: anti-human IFNγ-BV650, IL2-BV605 (BioLegend), IgG-BV421, TNFα-BUV395, CD69-BV750, CD71-BUV563, CD25-BV737 (BD Biosciences) Ki67-APC eF780 (ThermoFisher Scientific) in Perm/Wash. PBMCs were incubated at 4° C. in the dark for 30 minutes, washed twice in Perm/Wash buffer, once in FACS buffer, then re-suspended in 200 µL FACS buffer for acquisition on a custom four-laser Cytek Aurora spectral analyser using SpectroFlo v2.2 (Cytek biosciences).

Single-fluorochrome compensation was calculated on beads (BD Biosciences, Miltenyi) or human PBMCs. Analysis of data was conducted on FlowJo (v10.6.2) by a hierarchical gating strategy (figure S6) and Prism 8 (GraphPad). Peptide-specific responses were calculated by subtraction of the unstimulated controls from the peptide stimulated samples.

### MSD -Th1/Th2cytokine Profiling

Th1/Th2 cytokine responses were measured in tissue culture supernatants from the stimulation of PBMCs with synthetic peptides covering the spike protein. 5x10⁵ freshly isolated PBMCs were resuspended in 250µl of R10 media in 96 well U-bottom plates and supplemented with 1 µg/ml anti-human CD28 and CD49d. Peptides spanning the S1 and S2 subunits of the SARS-CoV-2 spike protein (table S1) were added to separate wells at a concentration of 2 µg/ml. Each sample also include an unstimulated (media only) control. Following a 16 - 18 hour incubation at 37° C. with 5% CO₂, cells were pelleted by centrifugation (1800 rpm, 5 min) and 200µl of supernatant was harvested. Supernatants from the S1 and S2 stimulations were combined and stored at -80° C. until required.

Cytokine responses were analysed using MSD (Meso Scale discovery) V-plex proinflammatory cytokine (human) Panel 1 kit, validated by MSD. Each plate is coated with 9 different capture mAbs against 9 different cytokines arranged in independent spots on the base of each well. Cytokines IFN-y, IL1b, IL-2, IL4, IL8, IL10, IL-12p70, IL13 and TNFα are associated with either a Th1 or Th2 type T-cell response.

Supernatants were diluted 1:2 for unstimulated sample and 1:10 for S1/S2 stimulated sample in using MSD diluent 2. The kit provides a multi-analyte lyophilised calibrator that when reconstituted will be used the standard curve using a 4-fold serial dilution to form an 8-point standard curve plated out in duplicate. Cytokine measurements were carried out according to manufacturer’s instructions. Plates are read on MSD reader within 15 mins of adding Read buffer. Data was analysed using MSD discovery workbench 4.0. Samples were repeated if any sample a replicate with a coefficient of variations (CV) greater than 20%. Replicates were read off the standard curve, multiplied by dilution factor, and concentration was reported as an average of the replicates in pg/ml. Concentration from unstimulated sample was subtracted from concentration from stimulated (background subtract). Negative values of background subtract have been replaced by zeros. An arbitrary value of 0.0001 has been added to the background subtracts across all the samples to overcome the presence of null values raised from samples too low to be read off the standard curve.

### Isotype & Subclass Standardised ELISA

Antigen specific total IgG was detected using an in-house indirect ELISA using trimeric SARS-CoV-2 spike protein, as described previously.

Standardised ELISA was used to quantify circulating SARS-CoV-2 spike-specific IgG1, IgG3, IgA and IgM responses. Nunc MaxiSorp™ ELISA plates (ThermoFisher Scientific) were coated overnight (≥16 hours) at 4° C. with 50 µL per well of 5 µg/mL SARS-CoV-2 full - length trimeric spike protein (FL-S) (The Jenner Institute, University of Oxford) diluted in PBS. A soluble SARS-CoV-2 FL-S protein (GenBank MN908947 Wuhan-Hu-1) construct encoding residues 1-1213 with two sets of mutations that stabilise the protein in a pre-fusion conformation (removal of a furin cleavage site and the introduction of two proline residues; K983P, V984P) was expressed as described⁶². The endogenous viral signal peptide was retained at the N terminus (residues 1-14), a C-terminal T4-foldon domain incorporated to promote association of monomers into trimers to reflect the native transmembrane viral protein, and a C-terminal His6 tag included for nickel-based affinity purification. FL-S was transiently expressed in Expi293™ (Thermo Fisher Scientific) and protein purified from culture supernatants by immobilised metal affinity followed by gel filtration in Tris-buffered saline (TBS) pH 7.4 buffer.

Plates were washed 3x with PBS/Tween (0.05%) (PBS/T) and tapped dry. Plates were blocked for 1 hour with 100 µL per well of Blocker™ Casein in PBS (ThermoFisher Scientific) at 20° C. Test samples were diluted in blocking buffer (minimum dilution of 1:50) and 50 µL per well was added to the plate in triplicate. For each immunoglobulin isotype or subclass being tested, the respective reference serum (made from a pool of high titre donor serum) was diluted in blocking buffer in a 2-fold dilution series to form a 10-point standard curve. 3 independent dilutions of the reference serum were made (with a dilution factor corresponding to the 4^{th} point in the standard curve) to serve as internal controls. The standard curve and internal controls were added to the plate at 50 µL per well in duplicate. Plates were incubated for 2 hours at 37° C. with 300 rpm shaking and then washed 3x with PBS/T and tapped dry. Secondary antibody was diluted in blocking buffer and 50 µL per well was added. The secondary antibody used was dependent on the immunoglobulin subclass or isotype being detected. These were Mouse Anti-Human IgG1 Hinge-AP, Mouse Anti-Human IgG3 Hinge-AP, Goat Anti-Human IgA-AP and Goat Anti-Human IgM-AP (Southern Biotech). Plates were incubated for 1 hour at 37° C. with 300 rpm shaking. Plates were washed 3x with PBS/T and tapped dry. 100 µL per well of PNPP alkaline phosphatase substrate (ThermoFisher Scientific) was added and plates were incubated for 1-4 hours at 37° C. with 300 rpm shaking. Optical density at 405 nm (OD₄₀₅) was measured using an ELx808 absorbance reader (BioTek) until the internal control reached an OD₄₀₅ of 1. The reciprocal of the internal control dilution giving an OD₄₀₅ of 1 was used to assign an ELISA unit (EU) value of the standard. Gen5 ELISA software v3.04 (BioTek) was used to convert the OD₄₀₅ of test samples into EUs by interpolating from the linear range of standard curve fitted to a 4-parameter logistics model. Any samples with an OD₄₀₅ below the linear range of the standard curve at the minimum dilution tested were assigned a minimum EU according to the lower limit of quantification of the assay.

### Isotype and Subclass OD ELISA

Antigen-specific IgG2, IgG4 and IgE responses were detected in the absence of an antigen-specific serum control. Nunc MaxiSorp™ ELISA plates (ThermoFisher Scientific) were coated with 50 µL per well of 5 µg/ mL SARS-CoV-2 trimeric spike protein (The Jenner Institute, University of Oxford). Plates were also coated with a specified concentration of a commercial human immunoglobulin control: recombinant Human IgG2 Lambda, recombinant Human IgG4 Lambda and recombinant Human IgE Lambda (Bio-Rad Laboratories Ltd). Plates were left overnight (≥16 hours) at 4° C. Plates were washed 3x with PBS/Tween (0.05%) (PBS/T) and tapped dry. Plates were blocked for 1 hour with 100 µL per well of Blocker™ Casein in PBS (ThermoFisher Scientific) at 20° C. Test samples and 5 pre-pandemic negative control samples were diluted 1:50 in blocking buffer and 50µL was added to antigen-coated wells in duplicate. 50 µL of blocking buffer was added to immunoglobulin-coated wells and blank wells. Plates were incubated for 2 hours at 37° C. with 300 rpm shaking and then washed 3x with PBS/T and tapped dry. Secondary antibody was diluted in blocking buffer and 50 µL per well was added. The secondary antibody used was dependent on the immunoglobulin subclass or isotype being detected: Mouse Anti-Human IgG2 Fd-AP, Mouse Anti-Human IgG4 Fc-AP and Mouse Anti-Human IgE Fc-AP (Southern Biotech). Plates were incubated for 1 hour at 37° C. with 300 rpm shaking. Plates were washed 3x with PBS/T and tapped dry. 100 µL per well of PNPP alkaline phosphatase substrate (ThermoFisher Scientific) was added and plates were incubated for 1-4 hours at 37° C. with 300 rpm shaking. Optical density at 405 nm (OD₄₀₅) was measured using an ELx808 absorbance reader (BioTek) until the immunoglobulin control reached a specified OD₄₀₅. Negative cut-offs were calculated using the formula Mean + 7.858*SD of the OD₄₀₅ readings of the pre-pandemic negative control serum samples, where 7.858 is the SD multiplier with a 99.9% confidence level for n=5 controls.

### Avidity ELISA

Anti-SARS-CoV-2 spike-specific total IgG antibody avidity of donor serum was assessed by sodium thiocyanate (NaSCN)-displacement ELISA. Nunc MaxiSorp™ ELISA plates (ThermoFisher Scientific) were coated overnight (≥16 hours) at 4° C. with 50 µL per well of 2 µg/ mL SARS-CoV-2 trimeric spike protein (The Jenner Institute, University of Oxford) diluted in PBS. Plates were washed 3x with PBS/Tween (0.05%) (PBS/T) and tapped dry. Plates were blocked for 1 hour with 100 µL per well of Blocker™ Casein in PBS (ThermoFisher Scientific) at 20° C. Test samples, and a positive control serum pool, we diluted in blocking buffer to normalise them to an OD₄₀₅ of 1 and 50 µL per well was added in duplicate to each row of the plate (except the last row where only blocking buffer was added). Plates were incubated for 2 hours at 20° C. and then washed 3x with PBS/T and tapped dry. Increasing concentrations of NaSCN (Sigma-Aldrich) diluted in PBS were added at 50 µL per well to each row down the plate (1 M, 2 M, 3 M, 4 M, 5 M, 6 M) except for the first and last row where only PBS was added. Plates were incubated for 15 minutes at 20° C. and then washed 6x with PBS/T and tapped dry. Anti-Human IgG (y-chain specific) -Alkaline Phosphatase antibody produced in goat (Sigma-Aldrich) was diluted 1:1000 in blocking buffer and 50 µL per well was added to the plate. Plates were incubated for 1 hour at 20° C. and then washed 3x with PBS/T and tapped dry. 100 µL per well of PNPP alkaline phosphatase substrate (ThermoFisher Scientific) was added and plates were incubated for 20° C. Optical density at 405 nm (OD₄₀₅) was measured using an ELx808 absorbance reader (BioTek) until the untreated sample wells reached an OD₄₀₅ of 1 (0.8-2.0). Gen5 ELISA software v3.09 (BioTek) was used to plot the test sample OD₄₀₅ against concentration of NaSCN and a spline function with smoothing factor 0.001 was fitted to the data. For each sample, concentration of NaSCN required to reduce the OD₄₀₅ to 50% of that without NaSCN (IC₅₀) was interpolated from this function and reported as a measure of avidity.

### Ex Vivo IFNγ ELISpot Assays

ELISpot assays were performed on freshly isolated PBMCs prior to, and 14 days after vaccination with ChadOx1 nCoV19. Assays were performed using Multiscreen IP ELISpot plates (Millipore) were coated overnight at 4° C. with 10 µg/ml of human anti-IFNγ coating antibody (clone 1-D1K, Mabtech) in carbonate buffer, before washing 3 times with PBS and blocking with R10 media for 2-8 hours. 2.5 x 10⁵ PBMCs were added to each well of the plate along with 13 pools of peptides covering the SARS-CoV-2 spike protein and the N-terminal tissue plasminogen activator leader sequence at a final concentration of 10 µg/ml (table S1). Each assay was performed in triplicate and incubated for 16 - 18 hours at 37° C. with 5% CO₂.

Plates were then developed by washing 6 times with PBS/T, followed by addition of 1 µg/ml anti-IFNγ detector antibody (7-B6-1-Biotin) to each well. After a 2 - 4 hour incubation, plates were washed again and 1:1000 SA-ALP added for 1-2 hours. After a final wash step, plates were developed using BCIP NBT-plus chromogenic substrate (Moss Inc.)

ELISpot plates were counted using an AID automated ELISpot counter (AID Diagnostika GmbH, algorithm C), using identical settings for all plates and spot counts were adjusted only to remove artefacts. Responses were averaged across triplicate wells and the mean response of the unstimulated (negative control) wells were subtracted. Results are expressed as spot forming cells (SFC)/10⁶ PBMCs. Responses to a peptide were considered positive if background subtracted responses were >40 SFU/10⁶ PBMCs. If responses were >80 SFC/10⁶ PBMC in the negative control (PBMC without antigen) or <800 SFC/10⁶ PBMC in the positive control wells (pooled Staphylococcal enterotoxin B at 0.02 µg/mL and phytohaemagglutinin-L at 10 µg/mL), results were excluded from further analysis.

### Intracellular Cytokine Staining

Intracellular cytokine staining (ICS) was performed on freshly isolated PBMCs stimulated with pooled S1 and S2 peptides. 3 x 10⁶ PBMCs were resuspended in 5 ml polypropylene FACS tubes to a volume of 1 ml in R10 media supplemented with 1 µg/ml anti-human CD28 and CD49d and 1 µl CD107a PE-Cy5 (eBioscience). S1 and S2 peptide pools (table S1) were added at a concentration of 2 µg/ml. Each sample also included a positive control (Staphylococcal enterotoxin B at 1 µg/ml, Sigma Aldrich) and an unstimulated (media only) control. Cells were incubated at 37° C. with 5% CO₂ for 16-20 hours with Brefeldin A (3 µg/ml) and monensin (2 mM) (eBioscience) added after 2 hours.

At the end of the incubation, cells were washed in FACS buffer (PBS containing 0.1% bovine serum albumin and 0.01% NaN₃) and transferred to a 96 well U-bottom tissue culture plate for staining. A surface staining cocktail was first added containing 2.5 µl of a 1:40 dilution of Aqua Live/Dead stain (ThermoFisher Scientific) and 1 µl of BV711 CCR7 (Biolegend) in 46.5 µl FACS buffer. Cells were incubated in the dark for 20 minutes and washed with FACS buffer. 100 µl CytoFix/CytoPerm solution (BD Biosciences) was added to each well and left to incubate for a further 20 minutes. Cells were then washed with Perm/Wash buffer before intracellular cytokine staining. The ICS cocktail contained 0.025 µl CD45RA BV605, 0.025 µl TNFα PE-Cy7, 0.1 µl IFNγ FITC, 0.025 µl CD14 e450, 0.025 µl CD19 e450, 0.5 µl CD3 AF700, 1 µl IL-2 BV650, 1.25 µl IL-5 PE, 2.5 µl IL-13 APC, 3.5 µl CD4 PerCP Cy5.5 and 5 µl CD8 APC-eF780 to a total volume of 50 µl diluted in FACS buffer. Samples were stained in the dark for 30 minutes. Cells were washed twice with perm/wash buffer and twice with FACS buffer before being resuspended in 100 µl of 1% paraformaldehyde.

Compensation controls were prepared fresh for each batch using OneComp eBeads (eBioscience). Cells were kept on ice and strained through a 35 µm filter before acquisition. Cells were acquired on a 5-laser BD LSRFortessa flow cytometer (BD Biosciences) and data analysed in FlowJo v10.7. A hierarchical gating strategy was applied for sample analysis (figure S7 A QC process was applied to remove samples with fewer than 100,000 events in the live CD3⁺ gate, samples with <1% cytokine response to SEB (CD4⁺ and CD8⁺ IFNγ, CD8⁺ TNFα). A lower limit of detection was applied and only samples with an ELISPOT response greater than 200 SFC/10⁶ PBMC were included in the analysis).

### Statistical Analysis

All statistical tests as well as all graphical representation of the data were performed in GraphPad Prism 8 (except for the correlation matrix scatterplot that was generated in SPSS Statistics 25). To check for the normality of the data d’Agostino-Pearson test was calculated. Descriptive comparison of immunoglobulin isotype/subclass levels applied medians with interquartile ranges (IQR); IgG avidity fold change by mean and standard deviation (SD). Unpaired samples were compared using a 2-tailed Mann-Whitney U or unpaired t test, depending on the distribution of the data. Correlations were analysed using Spearman’s rank test.

### Example 19: Expression of Native-Like SARS-CoV-2 Spike Glycoprotein by ChAdOxl nCoV-19

HeLa S3 cells were infected with ChAdOx1 nCoV-19 and incubated with either recombinant ACE2 or anti-ChAdOxl nCoV-19 (derived from vaccinated mice) and compared to non-infected controls, and analysed by flow cytometry. It was observed using flow cytometry that ChAdOx1 nCoV-19 produces membrane associated SARS-CoV-2 S glycoprotein in native conformations able to bind its host receptor ACE2.

Tomograms revealed that surface of the cells is densely covered with protruding densities consistent with the size and shape to the prefusion conformation of SARS-CoV-2 S protein (FIGS. 44A & 44B). These densities are absent in control uninfected cells. Cryo-immunolabelling using ChAdOx1 nCoV-19 vaccinated mice sera confirms the presentation of abundant S protein on the cell surface. We performed subtomogram averaging of 3274 spikes from cell surfaces using emClarity. The averaged density map is at 9.6 Å resolution (at 0.143 FSC cut-off), clearly resolving the overall spike structure, which overlaps very well with prefusion spike atomic models in the literature (FIGS. 44C & 44D). The subtomogram averaging, combined with the cytometry analysis and cryo-immunolabelling, unequivocally confirms that the majority of spike proteins on the surface are presented in the prefusion state.

High levels of glycan occupancy were observed across the protein. This is especially important since glycans are known to shield more immunogenic protein epitopes, thus the vector of the invention does not present epitopes that are not presented during natural infection which could divert the immune system from accessible neutralizing epitopes.

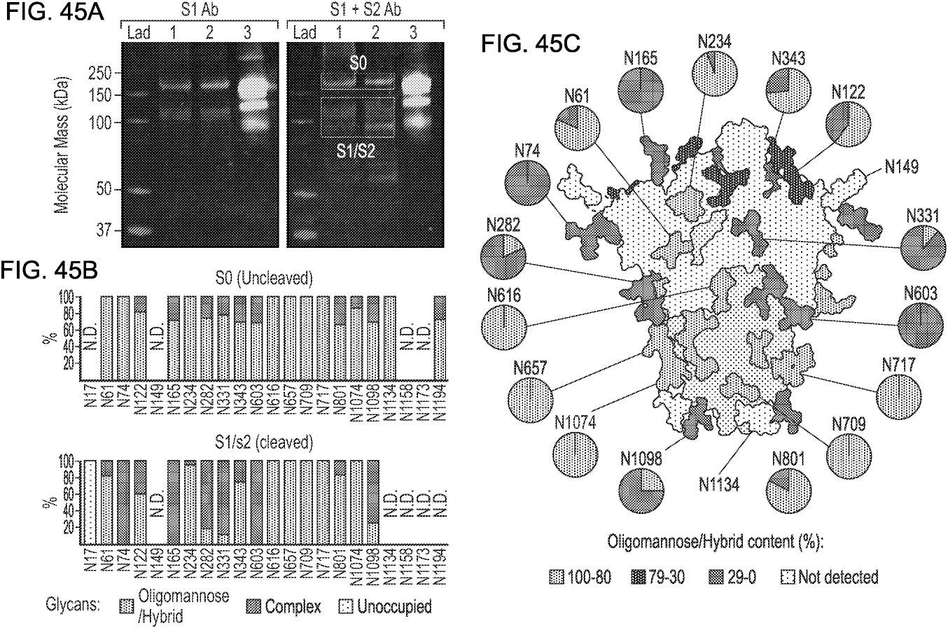

Using the cryo-EM structure of the trimeric SARS-CoV-2 S protein, we mapped the glycosylation status of the S1/S2 protein (FIG. 45C). A mixture of oligomannose/hybrid and complex-type sites were observed, with glycan sites such as N234, which are known to have stabilising effects on the RBD, preserving the predominantly oligomannose state reported in both recombinant proteins and viruses. Similarly, the glycan at N165 which also stabilises the RBD “up” conformation was determined to be complex-type on the S protein arising from infection of cells with ChAdOx1 nCoV19. Since glycans are sensitive reporters of local protein architecture, it is encouraging that such glycans, known to have structural roles, conserve their processing state which provides additional evidence of native-like prefusion protein structure.

Summary of Example 19: Conventionally, many vaccine candidates include stabilising mutations in the S protein, such that the protein maintains the prefusion conformation and avoids shedding of S1. In contrast, the viral vector of the invention does not comprise stabilising mutations in the S protein.

This example validates the structure, glycosylation and antigenicity of the S protein expressed from the viral vector ChAdOx1 nCoV-19/AZD1222. We demonstrate native-like post-translational processing and assembly, and reveal the expression of S proteins on the surface of cells adopting the trimeric prefusion conformation, which is especially advantageous as most neutralising antibodies target epitopes displayed on the prefusion spike. We show the native-like mimicry of SARS-CoV-2 S protein’s receptor binding functionality, prefusion structure, and processing of glycan modifications. It is a surprising advantage that these important native-like properties of the expressed S protein are obtained without the use of stabilising mutations.

### Example 20: Aged Subjects

We demonstrate that a single dose of ChAdOx1 nCoV-19 elicits a B and T cell response in 3-month-old adult mice, with formation of plasma cells, germinal centres and T follicular helper cells contributing to anti-spike antibody production. The development of humoral immunity is complemented by the formation of polyfunctional vaccine-specific Th1 cells and CD8⁺ T cells. In aged 22-month-old mice a single dose of ChAdOx1 nCoV-19 induced the formation of Th1 cells, vaccine-reactive CD8⁺ T cells, a germinal centre response and vaccine-specific antibodies. However, the cellular and humoral response was reduced in magnitude in 22-month-old mice compared to 3-month-old adult mice, with antibody isotypes and subclasses produced being of a similar profile. Administration of a second dose enhanced the germinal centre response and antibody titre in aged mice, and also boosted the numbers of granzyme B producing CD8⁺ T cells. Together, this indicates that the immunogenicity of ChAdOxl nCoV-19 can be enhanced in older individuals through the use of a prime-boost vaccination strategy.

Intramuscular immunisation drains antigen to the aLN and spleen resulting in the activation of antigen presenting cells in both compartments upon immunisation with ChAdOx1 nCoV-19 in mice, shown by examining immunofluorescence confocal images of DAPI expression and FluoSpheres™ (505/515) localisation in the aLN and spleen of mice immunised with yellow-green fluorescent FluoSpheres™ or PBS at 24 hr post intramuscular immunisation.

ChAdOx1 nCoV-19 induces a plasma cell and germinal centre B cell response as shown by tSNE/FlowSOM analyses of CD19⁺ B cells from 3-month-old (3 mo) mice seven days after immunization with ChAdOx1 nCoV-19 or PBS.

ChAdOx1 nCoV-19 induces a Th1 dominated CD4 cell response as shown by tSNE/FlowSOM analyses of CD4⁺ T cells from 3-month-old (3 mo) mice seven days after immunization with ChAdOx1 nCoV-19 or PBS.

ChAdOx1 nCoV-19 induces a CD8 T cell response as shown by tSNE/FlowSOM analyses of CD8⁺ T cells from 3-month-old (3 mo) mice seven days after immunization with ChAdOx1 nCoV-19 or PBS.

### A Prime-Boost Strategy Corrects Dysregulated CD8 T Cell Priming in Aged Mice

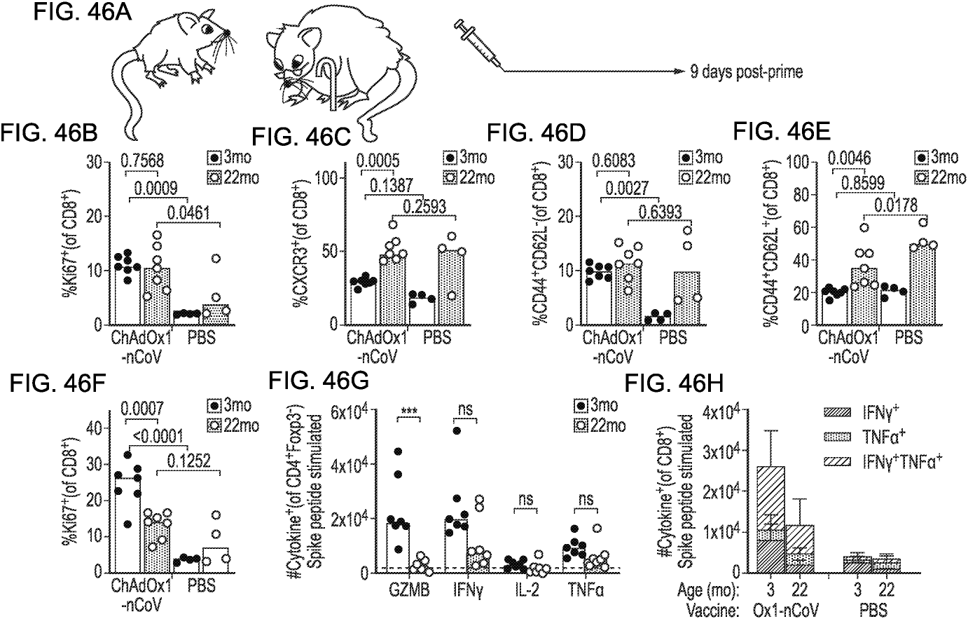

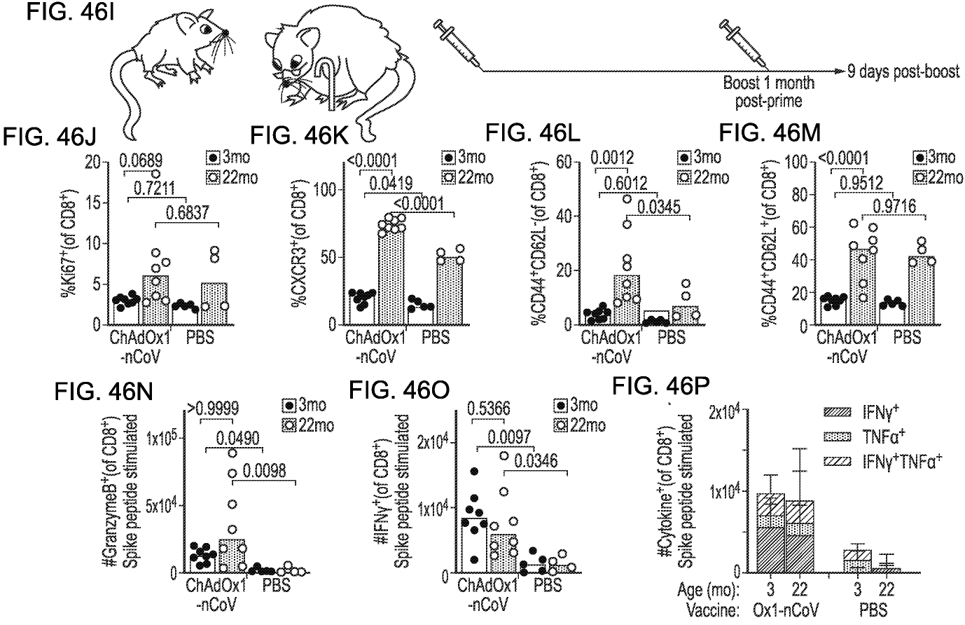

To assess the CD8⁺ T cell response to ChAdOx1 nCoV-19 immunisation in the context of ageing, we immunised 3-month-old and 22-month-old mice and enumerated the CD8⁺ T cell types altered by vaccination (in FIG. 4) nine days after immunisation (FIG. 46A). In the draining aLN, CD8⁺ T cells from aged mice expressed markers of activation and proliferation in response to ChAdOx1 nCoV-19. But the number of CXCR3⁺ cells or T effector memory cells did not increase in aged mice, when compared to the number in the PBS vaccinated group, as observed in young mice (FIGS. 46B-46D). At this early timepoint, the number of central memory T cells was not altered in either young or aged mice by ChAdOx1 nCoV-19 vaccination (FIG. 46E). In the spleen, fewer Ki67⁺ CD8⁺ T cells were observed in aged mice after ChAdOx1 nCoV-19 vaccination, compared to younger adult mice (FIG. 46F). The formation of antigen-specific CD8⁺ T cells was assessed by restimulating splenocytes with SARS-CoV-2 spike protein peptide pools. Aged mice had a stark defect in granzyme B producing CD8⁺ T cells, but production of IFNγ and TNFα was not significantly impaired compared to younger mice (FIG. 46G). IL-2 production was low in both adult and aged mice at this time point (FIG. 46G). Despite a trend to lower cytokine production by CD8⁺ T cells in aged mice, the proportion of polyfunctional CD8⁺ T cells was not significantly diminished in aged mice after ChAdOx1 nCoV-19 vaccination (FIG. 46H). This demonstrates that a single dose of ChAdOx1 nCoV-19 induces an altered CD8⁺ T cell response in aged mice characterised by a failure to form granzyme B-producing effector cells.

To determine whether a second dose could improve this response, we administered a booster dose of ChAdOx1 nCoV-19 one month after prime immunisation (FIG. 461). Nine days after boost, an increase in Ki67⁺ CD4 T cells was not observed in the draining aLN (FIG. 46J), possibly due to the kinetics of the secondary response being faster than the primary. A significant increase in CXCR3⁺ CD8⁺ T cells and effector memory cells was observed in aged mice after boost, with no change in the proportion of central memory cells in either age group (FIGS. 46K-46M). Assessment of antigen-specific splenocytes showed that the booster dose of ChAdOx1 nCoV-19 rescued the production of granzyme B producing CD8⁺ T cells in aged mice (FIG. 46N). IFNγ production and cytokine polyfunctionality are similar to that following prime immunisation (FIGS. 46O and 46P). This demonstrates that ChAdOx1 nCoV-19 is immunogenic in aged mice, and a booster dose can correct the age-dependent defect in the formation of granzyme B-producing, CXCR3⁺ and TEM CD8⁺ T cells.

### Prime-Boost Enhances the CD4⁺ T Cell Response to ChAdOx1 nCoV-19 in Aged Mice

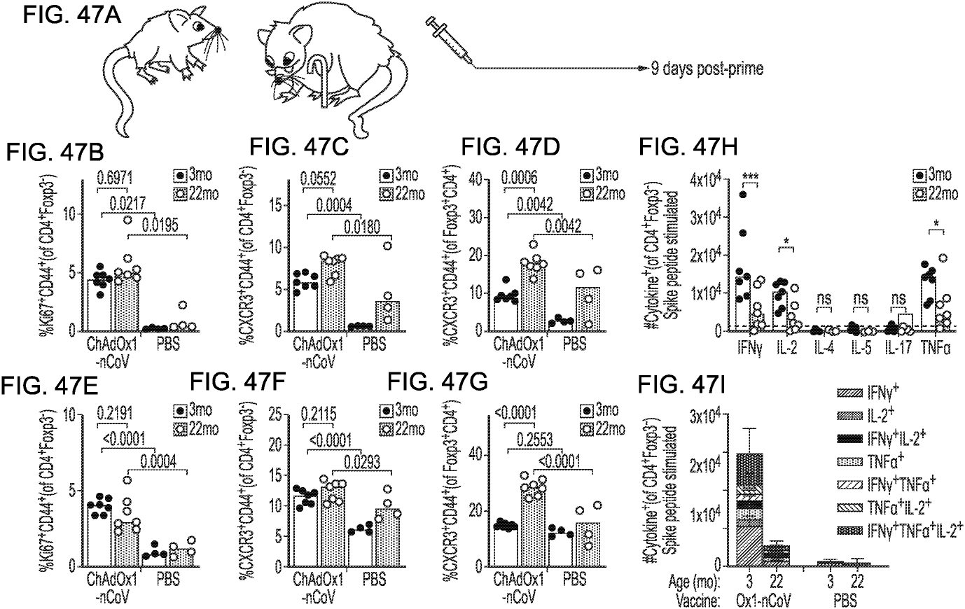

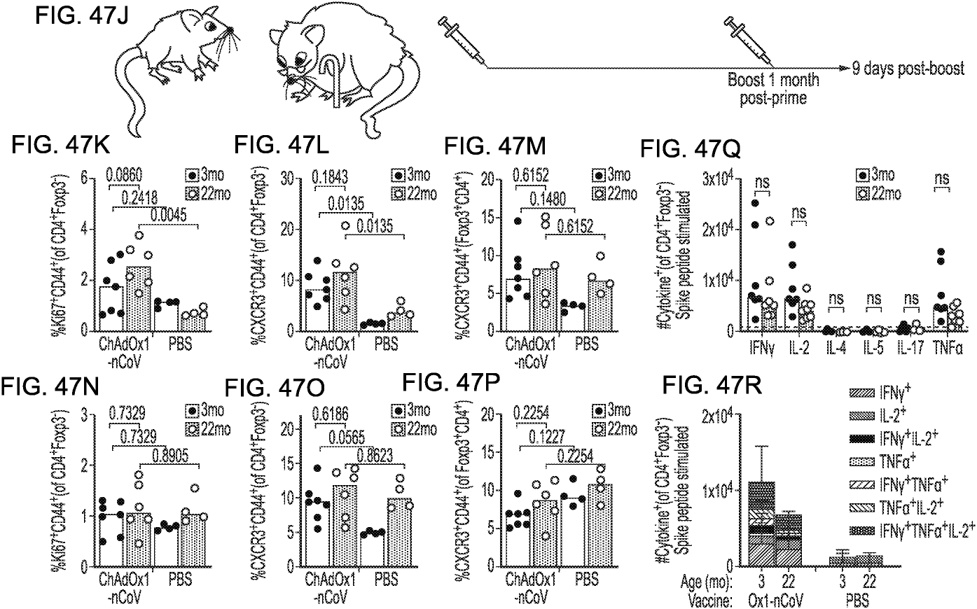

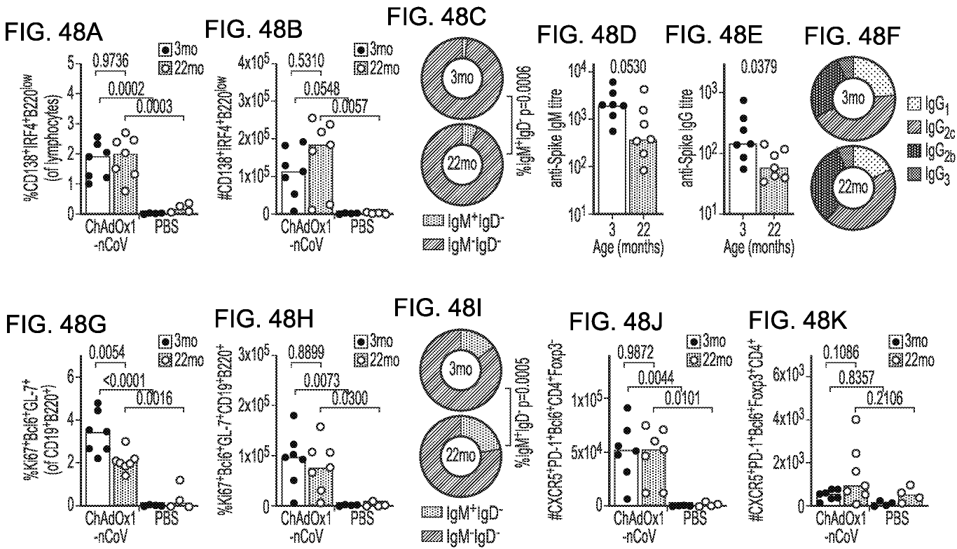

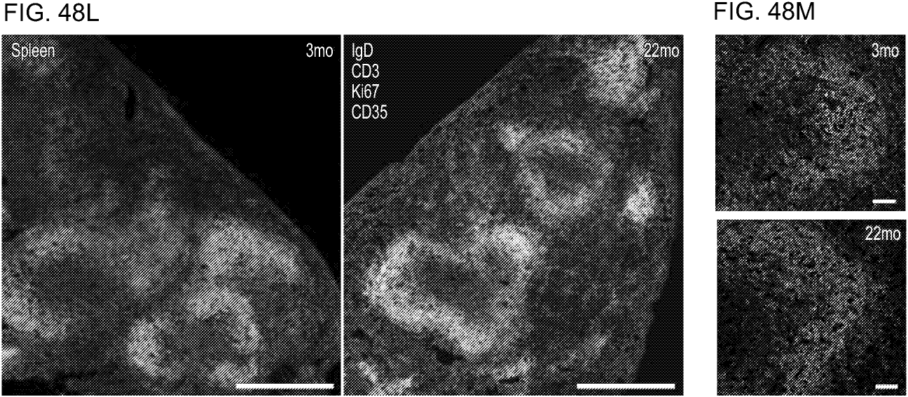

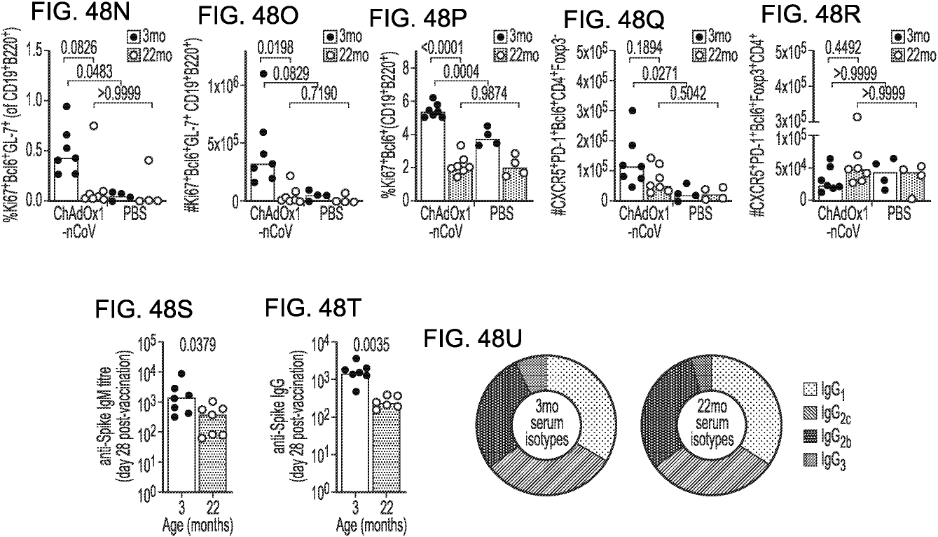

Nine days after primary immunisation of aged mice (FIG. 47A), an increase in Ki67⁺CD4⁺ T cells and CXCR3-expressing Th1 cells was observed in the draining lymph node of ChAdOx1 nCoV-19 immunised mice (FIGS. 47B and 47C). This was accompanied by an increase in Th1-like Tregs in both adult and aged mice (FIG. 47D). An increased frequency of these cell types was likewise observed in the spleen in response to ChAdOx1 nCoV-19 immunisation in both adult and aged mice (FIGS. 47E-47G). It is notable that, by these measurements, the response in aged mice is comparable to that in younger adults. The antigen-specific CD4⁺ T cell response was assessed by restimulating splenocytes with SARS-CoV-2 spike protein peptide pools. As in young mice, the response in aged mice to ChAdOx1 nCoV-19 was Th1 dominated, however there were fewer cytokine producing cells in aged mice nine days after a single immunisation (FIGS. 47H and 471). A booster dose of ChAdOx1 nCoV-19 administered one month after prime (FIG. 47J) stimulated Ki67 expression and the formation of CXCR3⁺CD44⁺ Th1 cells, but not CXCR3⁺ Th1-like Treg cells in the draining lymph node of aged mice (FIGS. 47K-47M). In the spleen, the booster dose did not enhance Ki67⁺ CD4⁺ T cells, or the formation of CXCR3⁺ conventional or regulatory T cells in adult or aged mice (FIGS. 47N-47P). In contrast to the response to primary immunisation, the number of antigen-specific cytokine producing cells was comparable in adult and aged mice after booster immunisation (FIGS. 48Q and 48R). Together, this indicates that the CD4⁺ T cell response to ChAdOx1 nCoV-19 immunisation is largely intact in aged mice, with a slight deficiency in antigen-specific cytokine production that can be enhanced by a booster immunisation.

### Aged Mice Have an Impaired Germinal Centre Response After Primary Immunisation

The majority of vaccines currently clinically available are thought to provide protection by eliciting humoral immunity, therefore it is important to quantify the B cell response to ChAdOxl nCoV-19 vaccination in the context of ageing. Early antibody production after vaccination arises from antibody-secreting cells generated in the extrafollicular plasma cell response, which is fast, but typically short-lived. A comparable early plasma cell response was detected in the aLN of younger adult and aged mice after immunisation (FIGS. 48A and 48B), although there was an increase in the proportion of non-class switched IgM⁺ plasma cells in aged mice (FIG. 48C). An intact plasma cell response was coupled with an increase in serum antibodies nine days after immunisation, which were of only slightly lower titre in aged mice, and of similar IgG subclass distribution to younger animals, again a predominantly Th1 dominated response (FIGS. 48D-48F).

Long-lived antibody-secreting cells typically arise from the germinal centre response. The percentage, but not total number, of germinal centre B cells was reduced in aged mice compared to younger adult mice after ChAdOx1 nCoV-19 vaccination (FIGS. 48G and 48H). Like the plasma cell response, there were more non-switched IgM⁺ germinal centre B cells in aged mice (FIG. 481). An increase in T follicular helper cells, but not T follicular regulatory cells, accompanied the lymph node germinal centre response in adult and aged mice (FIGS. 48J and 48K). In the spleen, germinal centres were easily visualised by microscopy in adult mice nine days after ChAdOx1 nCoV-19 vaccination, but were conspicuously absent in aged mice (FIG. 48L). Quantification of splenic germinal centres by flow cytometry confirmed impaired germinal centre formation in aged mice (FIGS. 48N and 48O). This was accompanied by fewer proliferating non-germinal centre B cells and T follicular helper cells in aged mice (FIGS. 48P and 48Q). As in the draining lymph node, splenic T follicular regulatory -cells were not induced by ChAdOx1 nCoV-19 vaccination at this time point (FIG. 48R). The impact of an impaired germinal centre response on vaccine-specific antibodies was observed 28 days after immunisation, with aged mice having lower titres of anti-spike IgM and IgG (FIGS. 48S and 48T), but a similar profile of IgG subclasses (FIG. 48U). Together, these data indicate that whilst a single of dose of ChAdOx1 nCoV-19 can induce comparable extrafollicular plasma cell responses between young and aged mice, the germinal centre response is compromised with age.

### A Second Dose of ChAdOx1 nCoV-19 Boosts Humoral Immunity in Aged Mice

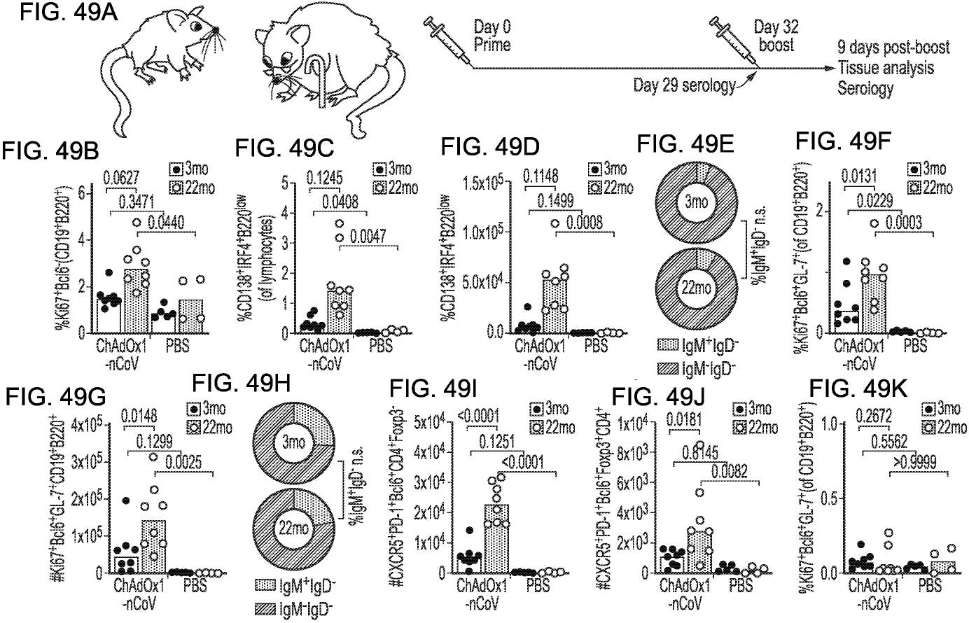

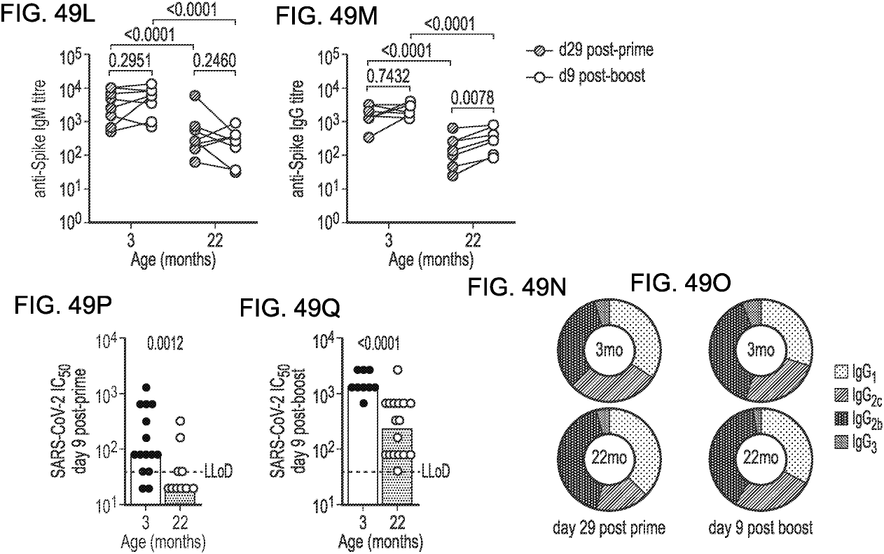

To test whether a prime-boost strategy can enhance the B cell response in aged mice, a prime-boost approach was taken (FIG. 49A). Nine days after boost, there were Ki67⁺ non-germinal enter B cells, plasma cells and germinal centre B cells in the draining lymph nodes of aged mice (FIGS. 49B-49H). Notably, the magnitude of the germinal centre response was larger in aged mice than in younger adult mice after boost (FIGS. 49F-49H) and this was associated with increased T follicular helper and T follicular regulatory cell numbers (FIGS. 491 and 49J). A germinal centre response was not observed in the spleen of either adult or aged mice nine days after booster immunisation (FIG. 49K). This demonstrates that a second dose of ChAdOx1 nCoV-19 can enhance the B cell response in aged mice. This improvement in the B cell response corresponded to an increase in anti-spike IgG, but not IgM, antibodies in every aged mouse that was given a booster immunization, without skewing IgG isotypes. The post boost ratio of IgG₂: IgG₁ was 2:1 in both younger adult and aged mice (FIGS. 49L-49O). The functional effect of the humoral immunity after both prime and boost immunisations was measured by SARS-CoV-2 pseudotyped virus microneutralization assay. Nine days after prime immunisation, SARS-CoV-2 neutralising antibodies were at levels lower in aged mice than measured in adult mice (FIG. 49P). Nine days after boost, neutralising antibodies were detectable in all aged mice and had been boosted eight-fold compared to early after the prime, although the titre was significantly lower than in younger adult mice (FIG. 49Q). This demonstrates that a booster dose of ChAdOx1 nCoV-19 can improve vaccine-induced humoral immunity in older mice.

### Mouse Housing and Husbandry

C57BL/6Babr mice were bred, aged and maintained in the Babraham Institute Biological Support Unit (BSU). No primary pathogens or additional agents listed in the FELASA recommendations⁶² were detected during health monitoring surveys of the stock holding rooms. Ambient temperature was ~19-21° C. and relative humidity 52%. Lighting was provided on a 12 hr light: 12 hr dark cycle including 15 min ‘dawn’ and ‘dusk’ periods of subdued lighting. After weaning, mice were transferred to individually ventilated cages with 1-5 mice per cage. Mice were fed CRM (P) VP diet (Special Diet Services) ad libitum and received seeds (e.g. sunflower, millet) at the time of cage-cleaning as part of their environmental enrichment. All mouse experimentation was approved by the Babraham Institute Animal Welfare and Ethical Review Body. Animal husbandry and experimentation complied with existing European Union and United Kingdom Home Office legislation and local standards (PPL: P4D4AF812). Young mice were 10-12 weeks old, and aged mice 93-96 weeks old when experiments were started. Mice that had tumours, which can occur in aged mice, were excluded from the analysis.

### Immunisation and Tissue Sampling

Mice were immunised in the right quadriceps femoris muscle with 50 µL of either 6x10⁹ virions of ChAdOx1 nCoV-19 in phosphate buffered saline (PBS) PBS alone, or 0.02 µm yellow-green fluorescent Carboxylate-Modified Microspheres (Invitrogen # F8787) in phosphate buffered saline. At the indicated timepoints post vaccination, blood, the right aortic lymph node, spleen and right quadriceps femoris muscle were taken for analysis.

### Enzyme-Linked Immunosorbent Assay

Standardised ELISA was performed to detect SARS-CoV-2 FL-S protein - specific antibodies in sera. MaxiSorp plates (Nunc) were coated with 100 or 250 ng/well FL-S protein overnight at 4° C. for detection of IgG or IgM and IgA, respectively, prior to washing in PBS/Tween (0.05% v/v) and blocking with Blocker Casein in PBS (Thermo Fisher Scientific) for 1 h at room temperature (RT). Standard positive serum (pool of mouse serum with high endpoint titre against FL-S protein), individual mouse serum samples, negative and an internal control (diluted in casein) were incubated for 2 h at RT for detection of specific IgG or 1 h at 37C for detection of specific IgM or IgA. Following washing, bound antibodies were detected by addition of AP-conjugated goat anti-mouse IgG (Sigma-Aldrich) for 1 h at RT or addition of AP-conjugated goat anti-mouse IgM or IgA (Abcam and Sigma-Aldrich, respectively) and addition of pNPP substrate (Sigma-Aldrich). An arbitrary number of ELISA units were assigned to the reference pool and OD values of each dilution were fitted to a 4-parameter logistic curve using SOFTmax PRO software. ELISA units were calculated for each sample using the OD values of the sample and the parameters of the standard curve.

The IgG subclass ELISA were performed according to the protocol described for detection of specific IgM or IgA in the serum. In addition, all serum samples were diluted to 1 total IgG ELISA unit and then detected with anti-mouse IgG subclass-specific secondary antibodies (Southern Biotech or Abcam). The results of the IgG subclass ELISA are presented using OD values instead of the ELISA units used for the total IgG ELISA.

### Micro Neutralisation Test Using Lentiviral-Based Pseudotypes Bearing the SARS-CoV-2 Spike

Lentiviral-based SARS-CoV-2 pseudotyped viruses were generated in HEK293T cells incubated at 37° C., 5% CO₂ as previously described (Graham, S.P. et al. Evaluation of the immunogenicity of prime-boost vaccination with the replication-deficient viral vectored COVID-19 vaccine candidate ChAdOx1 nCoV-19. npj Vaccines 5 (2020)). Briefly, cells were seeded at a density of 7.5 x 10⁵ in 6 well dishes, before being transfected with plasmids as follows: 500 ng of SARS-CoV-2 spike, 600 ng p8.91 (encoding for HIV-1 gag-pol), 600 ng CSFLW (lentivirus backbone expressing a firefly luciferase reporter gene), in Opti-MEM (Gibco) along with 10 µL PEI (1 µg/mL) transfection reagent. A ‘no glycoprotein’ control was also set up using the pcDNA3.1 vector instead of the SARS-CoV-2 S expressing plasmid. The following day, the transfection mix was replaced with 3 mL DMEM with 10% FBS (DMEM-10%) and incubated for 48 and 72 hours, after which supernatants containing pseudotyped SARS-CoV-2 (SARS-CoV-2 pps) were harvested, pooled and centrifuged at 1,300 x g for 10 minutes at 4° C. to remove cellular debris. Target HEK293T cells, previously transfected with 500 ng of a human ACE2 expression plasmid (Addgene, Cambridge, MA, USA) were seeded at a density of 2 x 10⁴ in 100 µL DMEM-10% in a white flat-bottomed 96-well plate one day prior to harvesting SARS-CoV-2 pps. The following day, SARS-CoV-2 pps were titrated 10-fold on target cells, and the remainder stored at -80° C. For micro neutralisation tests, mouse sera were diluted 1:20 in serum-free media and 50 µL was added to a 96-well plate in triplicate and titrated 2-fold. A fixed titred volume of SARS-CoV-2 pps was added at a dilution equivalent to 10⁵ signal luciferase units in 50 µL DMEM-10% and incubated with sera for 1 hour at 37° C., 5% CO₂ (giving a final sera dilution of 1:40). Target cells expressing human ACE2 were then added at a density of 2 x 10⁴ in 100 µL and incubated at 37° C., 5% CO₂ for 72 hours. Firefly luciferase activity was then measured with BrightGlo luciferase reagent and a Glomax-Multi⁺ Detection System (Promega, Southampton, UK). Pseudovirus neutralization titres were expressed as the reciprocal of the serum dilution that inhibited luciferase expression by 50% (IC₅₀).

### Statistics

All experiments were performed either twice or three times with 3-8 mice per group. Data was first tested for gaussian distribution using a Shapiro-Wilk test. Then data that was consistent with a normal distribution was analysed with either a student’s t-test for comparing two data set, or one-way ANOVA test for data with multiple groups. If the data did not follow a normal distribution then a Mann-Whitney test was used for comparing two data sets and a Kruskal Wallis test for multiple comparisons. All p-values shown are adjusted for multiple comparisons where multiple tests were performed on the same data. Analyses were performed within the Prism v8 software (GraphPad).

Summary of Example 20: The work presented here demonstrates that one dose of vector such as ChAdOx1 nCoV-19 is immunogenic in aged mice, but this response can be significantly improved with a second (booster) dose. Given that a second dose of ChAdOx1 nCoV-19 is immunogenic with expected reactogenicity profile in humans, this may be a viable strategy to enhance immunogenicity and possibly efficacy in older people.

### Example 21 - Effect on Virus Shedding

Ferrets are susceptible to infection with SARS-CoV-2. Following intranasal exposure of ferrets to SARSCoV-2 animals become infected and shed virus, detected by real-time PCR, for at least 9 days. The ferret model is considered to be an infection model for asymptomatic or mild human infections and an effective method of determining the efficacy of vaccine candidates by assessing reductions in virus shedding following virus exposure. This study, conducted by CSIRO, assessed the efficacy of a viral vector of the invention (ChAdOx1 nCoV-19) against SARS-CoV-2 in a ferret challenge model. The ChAdOx1 nCoV-19 composition was assessed by two different routes of administration (intranasal and intramuscular) with ferrets receiving either one or two doses of the composition. The dose amount was dose of 2.5 x 10¹⁰ virus particles per ferret in 100 µL PBS.

### Test System

Outbred ferrets, previously vaccinated against Canine distemper virus (canine Protech C3 vaccine; 2 doses) were used as they are susceptible to SARS-CoV-2 infection. SARS-CoV-2 -BetaCoV/Australia/VIC01/2020; Lot No. 2002-03-1628 was used as the challenge inoculum in this study. The growth and characterisation of this challenge inoculum was performed by CSIRO.

### Study Design

This was a randomised, placebo-controlled study assessing the ChAdOx1 nCoV-19 vaccine against a control (PBS, placebo). The vaccine was assessed by two different routes of administration, Intranasal (IN) and Intramuscular (IM). The study was carried out as a block design with two cohorts of 20 animals (total of 40 animals, 20 males and 20 females). The dosing regime is outlined in the Table below:

| Study Cohort | Study Group | No. of Ferrets | Study Items | Day 0 | Day 28 | Virus Challenge¹ | Study End² |
|---|---|---|---|---|---|---|---|
| 1 | 1 | 8 (4 M & 4 F) | ChAdOx1 nCoV-19 vaccine IM | Yes | No | Day 28 SARS-CoV-2 | Day 42 |
| | 2 | 8 (4 M & 4 F) | ChAdOx1 nCoV-19 vaccine IN | Yes | No | | |
| | 4 | 4 (2 M & 2 F) | Reference Item 1.0 mL PBS | Yes | No | 3 x 10⁴ TCID50 in | |
| 2 | 5 | 8 (4 M & 4 F) | ChAdOx1 nCoV-19 vaccine IM | Yes | Yes | Day 56 SARS-CoV-2 3 x 10⁴ TCID50 in 0.5 mL | Day 70 |
| | 6 | 8 (4 M & 4 F) | ChAdOx1 nCoV-19 vaccine IN | Yes | Yes | | |
| | 8 | 4 (2 M & 2 F) | Reference Item 1.0 mL PBS | Yes | Yes | | |

Notes:
¹Target challenge dose based on characterised titre of challenge virus stock
²Study end is 14 days post challenge.

At challenge (Cohort 1 - Day 28; Cohort 2 - Day 56), all ferrets were intranasally exposed to a target of 3 x 10⁴ 50% Tissue Culture Infectious Dose (TCID50) of SARS-CoV-2.

Following challenge, ferrets were observed for the onset and progression of disease, with all ferrets sampled in Cohort 1 on Days 31, 33, 35, 37 and 39 and, Cohort 2 on Days 59, 61, 63, 65 and 67.

On Day 42 (Cohort 1) or Day 70 (Cohort 2) ferrets had blood and shedding samples collected and then between Days 43-45 (Cohort 1) or Days 71-73 (Cohort 2) ferrets were humanely killed and a range of samples collected at necropsy for analysis.

### Determination of Viral RNA Load in Samples

The viral RNA load was determined for all tissue samples, swabs and nasal wash samples following RNA extraction and analysis by quantitative real-time polymerase chain reaction (qRT-PCR), with testing for detection of SARS-CoV-2 RNA performed in duplicate reactions. Primer and Probe Sequences for Coronavirus qRT-PCR Assays are in the Table below:

| | |
|---|---|
| Primer/Probe Name | Sequence |
| CoV E fwd (SEQ ID NO: 22) | 5′- AGTACGAACTTATGTACTCATTCGTT -3′ |
| CoV E rev (SEQ ID NO: 23) | 5′- ATATTGCAGCAGTACGCACACA -3′ |
| CoV probe (SEQ ID NO: 24) | 5′- 6FAM ACA CTA GCC ATC CTT CG MGBNFQ -3′ |

Copy numbers for individual samples were calculated using cycle threshold (CT) values as SARS-CoV-2 E gene copies per mL or per g, with an equation established from standard curve data using a synthetic DNA standard of known copy number concentration.

### Determination of Infectious Viral Load in Samples

Samples testing positive by qRT-PCR with a CT value of less than 35 were further analysed by TCID50.

### qRT-PCR Testing of Swab and Nasal Wash Samples

All nasal wash and swab samples collected at the pre-challenge sampling event (Day 24 - Cohort 1; Day 52 - Cohort 2) had undetectable levels of CoV E RNA, as tested by qRT-PCR (QA/23-2-43). RNA copy numbers (Log10 CoV E copies/mL) for each sample collected post-challenge are shown in FIG. 8 to FIG. 11 for Days 3, 5, 7, 9 post challenge (Days 31, 33, 35 and 37 - Cohort 1; Days 59, 62, 63 and 65 - Cohort 2). No virus shedding was detected on Days 11 or 14 from any ferret (Days 39 and 42 - Cohort 1; Days 67 and 70 - Cohort 2).

Statistical analysis was performed for each of the Study Groups against the pooled controls (Study Groups 4 and 8). P values for unpaired t-tests are listed in the Table below: Statistical Significance of Viral RNA Loads in Study Groups Compared to Pooled Controls:

Nasal wash

| Day post challenge | Study Group | | | |
|---|---|---|---|---|
| | Group 1 - IM prime only | Group 5 - IM prime and boost | Group 2 - IN prime only | Group 6 - IN prime and boost |
| Day 3 | 0.5368 | 0.2898 | 0.3165 | 0.0292 |
| Day 5 | 0.1034 | 0.0247 | 0.3199 | 0.0036 |
| Day 7 | 0.0282 | 0.0156 | 0.0149 | 0.0097 |
| Day 9 | 0.1379 | 0.1676 | 0.1379 | 0.1379 |

Oral swab

| Day post challenge | Study Group | | | |
|---|---|---|---|---|
| | Group 1 - IM prime only | Group 5 - IM prime and boost | Group 2 - IN prime only | Group 6 - IN prime and boost |
| Day 3 | 0.0275 | 0.1072 | 0.0438 | 0.0116 |
| Day 5 | 0.0189 | 0.0118 | 0.2230 | 0.0071 |
| Day 7 | 0.2643 | 0.2997 | 0.2643 | 0.2643 |
| Day 9 | 0.2643 | 0.2997 | 0.2643 | 0.2643 |

Notes: Bold p values are statistical significance in an unpaired t test (p < 0.05).
As no viral RNA was detected on Days 11 and 14 post challenge, no statistical analysis

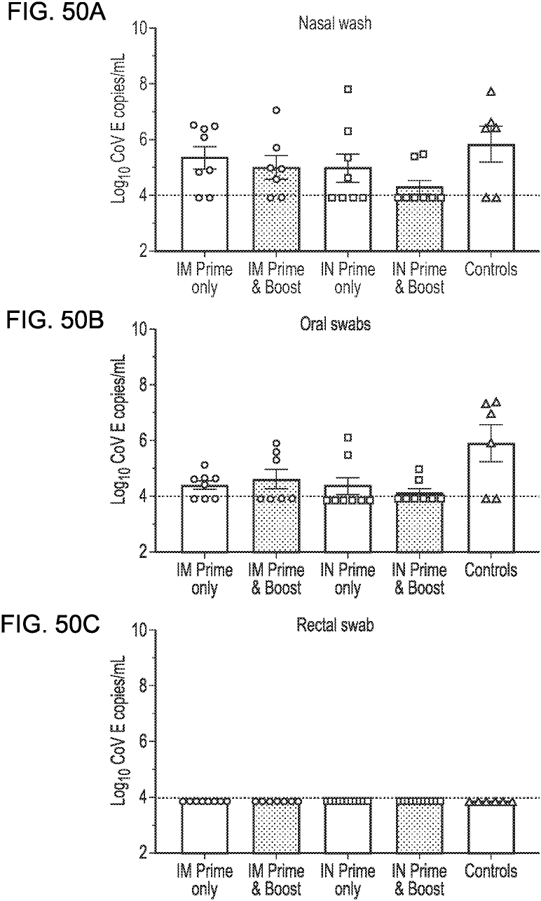

FIG. 50 shows bar charts of viral load in nasal wash and rectal and oral swab fluids measured by qRT-PCR on Day 3 post challenge (Study Day 31 - Cohort 1 or 59 - Cohort 2).

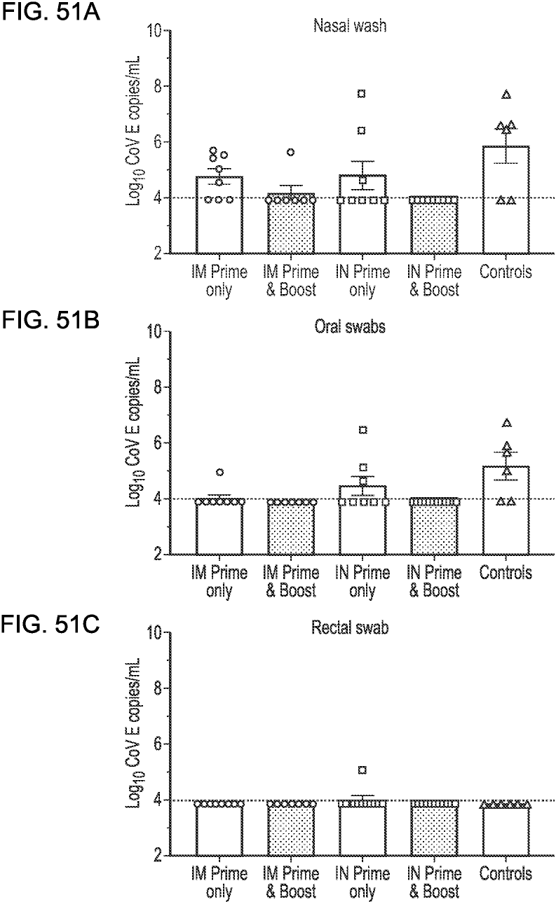

FIG. 51 shows bar charts of viral load in nasal wash and rectal and oral swab fluids measured by qRT-PCR on Day 5 post challenge (Study Day 33 - Cohort 1 or 61 - Cohort 2) analysed.

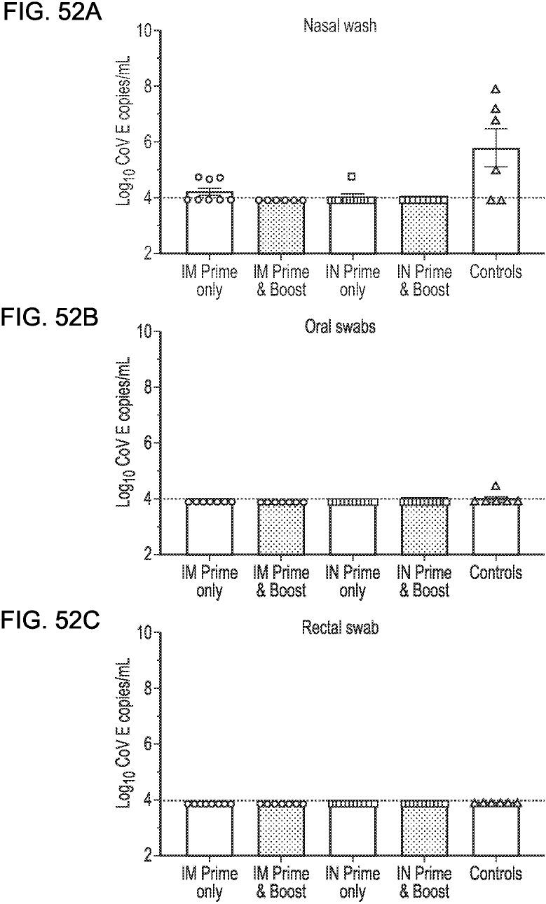

FIG. 52 shows bar charts of viral load in nasal wash and rectal and oral swab fluids measured by qRT-PCR on Day 7 post challenge (Study Day 35 - Cohort 1 or 63 - Cohort 2).

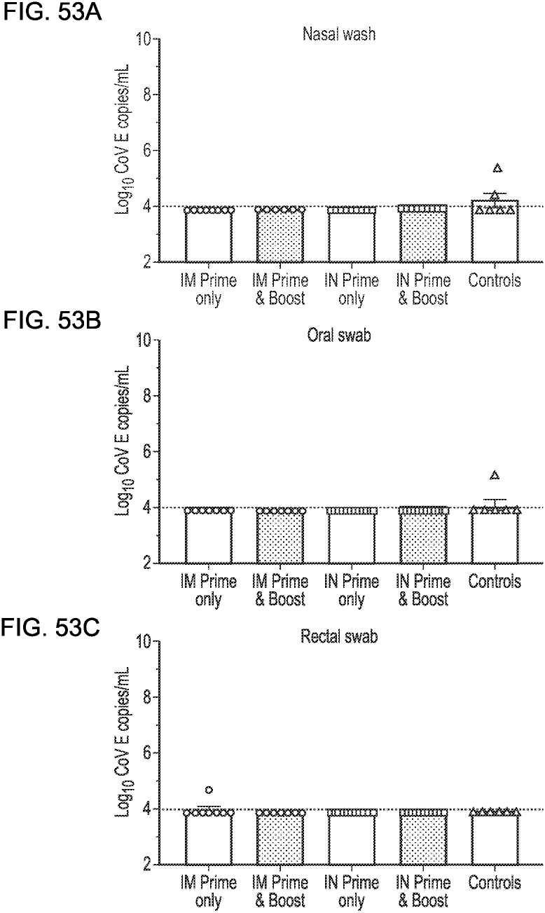

FIG. 53 shows bar charts of viral load in nasal wash and rectal and oral swab fluids measured by qRT-PCR on Day 9 post challenge (Study Day 37 - Cohort 1 or 65 - Cohort 2).

Notes for FIGS. 50-53: RNA was extracted from samples analysed in duplicate using qRT-PCR assay detecting CoV E RNA. Black points represent mean titre of the duplicate reactions for individual ferrets, bars are the mean titres for each group, error bars represent standard error of the mean and the dotted lines are the limit of detection of the assay. Where no viral RNA was detected by qRT-PCR, a data point has been plotted at 3.9.

Summary of Example 21: This example shows a decrease in average virus shedding, detected by qRT-PCR from nasal wash, oral swab and rectal swab samples, in all study groups of ferrets that were administered the composition of the invention (vaccine) compared to controls at the timepoints assessed post challenge. These reductions were statistically significant in nasal wash and swab titres at various timepoints. A prime/boost regimen achieved better reductions in shedding compared to a single administration.

### Example 22: Safety and Efficacy of the ChAdOxl nCoV-19 Vaccine (AZD1222) Against SARS-CoV-2

Methods. We describe phase III efficacy trials of ChAdOx1 nCoV-19 in the United Kingdom and Brazil, and phase I/II clinical trials in the UK and South Africa which include an efficacy cohort. All studies contribute here to a pooled safety analysis. The phase III efficacy cohorts in UK and Brazil contribute to efficacy assessments. In the efficacy cohorts, participants over 18 years of age are randomised 1:1 to receive two doses of ChAdOx1 nCoV-19 or a control/placebo (a meningococcal conjugate vaccine, MenACWY, in the UK; saline in South Africa; and, in Brazil, MenACWY for the first dose and saline for the second). Participants in the ChAdOx1 nCoV-19 group received doses containing ~5x10¹⁰ viral particles (standard dose: SD), except a subset who received a half dose as their first dose in the UK (low dose: LD). The primary endpoint is symptomatic COVID-19 disease, defined as having a nucleic acid amplification test (NAAT) positive swab with at least one of the following symptoms: fever ≥ 37.8° C.; cough; shortness of breath; anosmia or ageusia. Each NAAT positive case was assessed by a blinded independent endpoint review committee who also classified the severity of each case according to the WHO clinical progression scale. The primary analysis included only participants who were seronegative at baseline and had symptomatic NAAT positive COVID-19 disease > 14 days after the second dose of vaccine. A secondary analysis included cases occurring more than 21 days after the first standard dose vaccine in those who received one or two standard doses. For measurement of efficacy against asymptomatic disease, asymptomatic cases were identified by use of routine weekly swabbing in the UK.

Vaccine efficacy was calculated as 1 - relative risk derived from a robust Poisson regression model adjusted for age at randomisation. Alpha of 4.16% was used for the interim analysis. Serious adverse events were collected throughout the study period.

The studies are ongoing, and are registered at ISRCTN89951424 and ClinicalTrials.gov, NCT04324606, NCT04400838, NCT04444674.

Findings: Since the initiation of phase I trials in the UK on 23^{rd} April 2020, a total of 24,103 participants have been enrolled in clinical trials of ChAdOx1 nCoV-19 in the three countries, 11829 in the UK, 2096 in South Africa and 10178 in Brazil. Of these 11636 were eligible for inclusion in the primary analysis of efficacy >14 days after the second dose of vaccine (7548 in UK, 4088 in Brazil). There were 30 symptomatic COVID-19 cases in the ChAdOx1 nCoV-19 vaccinated group and 101 cases in the control group. Vaccine efficacy more than 14 days after the second dose was 70.4% (95.8% CI 54.8%, 80.6%). Vaccine efficacy was for those receiving LD/SD was 90.0% (95% CI 67.4%, 97.0%) compared with 60.3% (28.0%, 78.2%) in those receiving SD/SD.

From 21 days after the first dose there were 10 cases hospitalised for COVID-19, and two were classified as severe. All 10 were in the control arm of the study. Vaccine efficacy for these endpoints was not computed due to small numbers. There was one death due to COVID-19, in the control arm of the study.

Efficacy against asymptomatic infection in the UK was 27.3% (95% CI -17%, 55%) for the primary analysis cohort, and was 58.9% (95% CI 1.0%, 82.9%) among those receiving LD/SD and 3.8% (-72.4%, 46.3%) in those who received SD/SD.

In a safety cohort that included 73480 person-months of follow up, there were 144 serious adverse events (SAEs) reported that were considered unrelated to any of the study interventions. One SAE was assessed as possibly vaccine-related in the ChAdOx1 nCoV-19 vaccine group and one in the control group. A case of severe fever was also reported as vaccine-related.

Interpretation: ChAdOx1 nCoV-19 has an acceptable safety profile and is efficacious against symptomatic COVID-19 disease in this interim analysis of an ongoing clinical trial

### Detail

Following initiation of a phase I clinical trial in the UK (COV001) on 23rd April 2020, three further randomised controlled trials of the candidate vaccine were initiated across the UK (COV002), Brazil (COV003), and South Africa (COV005). The phase I study (COV001) included an efficacy cohort and the phase II and III studies (COV002, COV003, and COV005) expanded enrolment to a wider population of participants with higher likelihood of exposure to the virus, such as healthcare workers. Exclusion criteria were reduced for phase III, and older adults with a range of co-morbidities were also enrolled. All studies have essentially completed enrolment of their respective efficacy cohorts and are in the follow-up phase, with the exception of a small number of volunteers still being recruited into a HIV positive cohort in the UK, and older adults in Brazil. Here we present the combined interim analysis of vaccine efficacy against symptomatic COVID-19 infection from these four randomised controlled trials of ChAdOx1 nCoV-19.

### Methods

The safety and efficacy of the ChAdOx1 nCoV-19 vaccine is being assessed by a global pooled analysis that incorporates data from four ongoing phase I, II, and III clinical studies of the vaccine. The pooled analysis of data from these studies provides greater precision for both efficacy and safety outcomes than can be achieved in individual studies and provides a broader understanding of the use of the vaccine in different populations. A statistical analysis plan for the global pooled analysis of these studies was developed prior to data lock and analysis.

### Cov001 (UK)

In this ongoing large phase I/II clinical trial in 5 sites in the UK, we enrolled 1077 healthy volunteers aged 18-55 years as described above. Briefly, healthy adult participants were enrolled after screening to exclude those with pre-existing health conditions. Participants in efficacy cohorts were randomised 1:1 to receive 2 doses of ChAdOx1 nCoV-19 at a dose of 5 x 10¹⁰ viral particles (vp), or MenACWY as control. This study was originally planned as a single dose study, but the protocol was modified to a 2 dose regime for the efficacy cohorts as a result of robust booster responses identified in the evaluation of the early immunogenicity cohorts.

### Cov002 (UK)

In this ongoing single-blind phase II/III study in the UK, 19 study sites in England, Wales and Scotland enrolled participants, particularly targeting those working in professions with high possible exposure to SARS-CoV-2, such as health and social care settings. Participants were enrolled into efficacy cohorts (groups 4, 6, 9, 10) in three age groups, 18-55 years, 56-69 years and 70 years and older, with no upper age limit. We included individuals with stable pre-existing health conditions, and aimed to enrol approximately 20% of participants to the two older age bands. Participants enrolled into immunogenicity subgroups have been described above and/or previously published and are not included further in this example. Two dose levels were included in the UK trial: in the LD/SD group participants received a half dose of the vaccine (~2.5 x 10¹⁰ vp) as their first dose (LD) and were boosted with a standard dose (SD); subsequent cohorts were vaccinated with two standard-dose vaccines (SD/SD). Initial dose selection was based on the same spectrophotometry measurement used in the phase I study (COV001), but, as a result of a difference in manufacturing process, this was subsequently shown to under-estimate the dose resulting in a half dose (LD), and the dose was adjusted using qPCR to SD resulting in enrolment of 2 vaccine groups with different dosing regimens. Full details are available in the protocol.

### COV003 (Brazil)

In this ongoing single-blind phase III study in Brazil the focus of recruitment was targeted to those at high risk of exposure to the virus, including healthcare workers at 6 sites across Brazil. This trial included individuals with stable pre-existing health conditions. All participants were offered two doses of the vaccine at a dose of ~5 x 10¹⁰ vp administered 4 weeks apart. Participants were recruited across all age groups over 18 years of age with approximately 20% over 55 years of age. Full details are available in the study protocol.

### COV005 (South Africa)

This ongoing study in South Africa is a double-blind phase I/II study in healthy adults aged 18-65 years living without HIV. An additional immunogenicity cohort of those living with HIV was also enrolled but are not included in this report. All participants were offered two doses of the vaccine at a dose of 5 x 10¹⁰ vp, with doses administered 4 weeks apart (median and range). A small subgroup of 44 participants received a low dose vaccine. Full details are available in the study protocol

### Randomisation

In efficacy cohorts for all studies, participants were randomised 1:1 to receive ChAdOx1 nCoV-19 or a control product. Randomisation lists were prepared by the study statistician and uploaded into to the secure web platform used for the study eCRF (REDCap 9.5.22 - © 2020 Vanderbilt University) for COV001, COV002, and COV003. In South Africa the randomisation list was held by the unblinded study pharmacist who prepared the vaccines for administration. Vaccine syringes were covered with an opaque material to prevent unblinding of study participants.

### Vaccines Administered

The ChAdOx1 nCoV-19 vaccine is a replication-deficient simian adenoviral vector expressing the full-length spike (S) protein of SARS-CoV-2. In COV002 meningococcal Group A, C, W and Y conjugate vaccine (MenACWY), was chosen as the control group vaccine to minimise the chance of accidental participant unblinding due to local or systemic reactions to the vaccine. COV003 used MenACWY as the control for the first dose and saline for the second dose. In COV005, participants randomised to the control group were administered saline solution.

COV001, COV002 and COV003 were initially designed to assess a single-dose of ChAdOx1 nCoV-19 compared with control. However, after review of the antibody response data from the phase ½ study (COV001) and in the immunogenicity cohort in older adults in phase II which showed an increase in total and neutralising antibody titres after a booster dose, a booster dose was incorporated into all trials.

### Endpoint Ascertainment

### Symptomatic COVID-19 Disease

Participants were asked to contact the study site if they experienced specific symptoms associated with COVID-19 and received regular reminders to do so. Those who met symptomatic criteria had a clinical assessment, a swab taken for NAAT, and blood samples taken for safety and immunogenicity. In the UK and Brazil the list of qualifying symptoms for swabbing included any one of the following: fever ≥ 37.8° C.; cough; shortness of breath; anosmia or ageusia. In South Africa the list of qualifying symptoms for swabbing was broader, and included myalgia, chills, sore throat, headache, nasal congestion, diarrhoea, runny nose, fatigue, nausea, vomiting, and loss of appetite.

In all studies, if participants were tested outside the trial, either in their workplace if a health care worker, or by private providers, these results were recorded and assessed by an independent endpoint review committee. The source of each swab was recorded plus the details of the test kit where available.

### Asymptomatic COVID-19 Infection

Participants in COV002 in the UK were asked to provide a weekly self-administered nose/throat swab for NAAT testing from 1 week after first vaccination using kits provided by the Department of Health and Social Care (DHSC). Swabs were taken by participants in their home and posted to dedicated DHSC testing laboratories for processing. Participants were directly informed of their results by text or email from the National Health Service (NHS). Swab results from English and Welsh participants were provided to the trial statistician on a daily basis by the NHS and matched to individuals based on personal identification data (name, date of birth, NHS number, postcode). Swab results from Scottish NHS participants were unavailable to the study team at the time of the data cut-off date for this analysis. Any swab results that were not able to be matched to a study participant using at least two pieces of personal data were not added to the study database.

### Endpoint Review

All cases were reviewed by two members of a blinded independent clinical review team who assessed clinical details including medical history, symptoms, adverse events, and swab results, and assigned severity scores according to the World Health Organisation clinical progression scale (Marshall JC, Murthy S, Diaz J, et al. A minimal common outcome measure set for COVID-19 clinical research. The Lancet Infectious Diseases 2020).

### Inclusion in the Analysis

Participants in all studies are included here in safety tables. However, each study had to meet prespecified criteria of having at least 5 cases eligible for inclusion in the primary outcome before a study was included in efficacy analyses. Neither COV001 or COV005 met these criteria and so are not included in efficacy assessment for this interim analysis.

### Statistical Methods

The plan for assessing efficacy and safety for the ChAdOx1 nCoV-19 vaccine is based on global analyses utilizing all available data from four studies with analysis pooled across the studies. A global statistical analysis plan for pooling study data was developed, after extensive advice from regulators, to pre-specify the analyses that would contribute to the assessment of efficacy and this was signed off prior to any data analysis being conducted.

Vaccine efficacy was calculated as 1 - the adjusted relative risk (ChAdOx1 nCoV-19 vs control groups) computed using a Poisson regression model with robust variance (Zou G. A modified poisson regression approach to prospective studies with binary data. American journal of epidemiology 2004; 159(7): 702-6). The model contained terms for study, treatment group, and age group at randomisation. A reduced model which did not contain a term for age was used for models affected by convergence issues due to having few cases in the older age groups. The logarithm of the period at risk for primary endpoint for pooled analysis was used as an offset variable in the model to adjust for volunteers having different follow up times during which the events occurred.

The global pooled analysis plan allowed for one interim and a final efficacy analysis with alpha adjusted between the two using a flexible gamma alpha-spending function, with significance being declared if the lower bound of the 1-α% confidence interval is greater than 20%. Evidence of efficacy at the time of the interim analysis was not considered reason to stop the trials and all trials are continuing to accrue further data which will be included in future analyses.

The first interim analysis was planned to be triggered when at least 53 cases in participants who received two standard dose vaccines (SD/SD) had accrued that met the primary outcome definition more than 14 days after the second dose. This analysis provides 77% power for the 20% threshold to assume a true vaccine efficacy of 70%. Due to the rapid increase in incidence of COVID-19 in the UK in October combined with delays in shipping of baseline samples for assessment of antibodies to SARS-CoV-2 at the time of vaccination, an analysis at 53 cases was not achievable. By the time of data lock for this interim analysis, 98 cases were available for inclusion in the SD/SD cohorts and based on these numbers, the alpha level calculated using the gamma alpha spending function for this analysis is 4.16%.

Participants were excluded from the primary efficacy analysis if they were seropositive at baseline or had no baseline result. Serum samples were measured at baseline in a validated serological assay using the nucleocapsid antigen of SARS-COV-2 and run at PPD Central Labs (Zaventum, Belgium and Highland Heights, KY, USA). The Roche Elecsys Anti-SARS-CoV-2 serology test is an electroluminescence immunoassay-based modality that allows for the qualitative detection of IgG reactive to the SARS-CoV-2 nucleoprotein in human sera. Other exclusions included those with NAAT-positive swabs before 15 days after the second vaccination, or those who discontinued from the study prior to having met the primary efficacy endpoint and their follow-up time was less than 15 days after the second vaccination.

An analysis of efficacy after a first SD vaccine was undertaken as a secondary analysis. Individuals were excluded if they had a NAAT-positive swab before 22 days after a first dose given as SD, or if the first dose was LD.

Participants were analysed according to the vaccines they received, with a sensitivity analysis conducted as intention-to-treat.

Data analysis was done using R version 3.6.1 or later. Robust Poisson models were fitted using “proc genmod” function in SAS version 9.4. The alpha level for the analysis was calculated using the “gsDesign” function in R.

### Results

There were 24103 participants who were recruited and vaccinated (1077 UK (COV001), 10752 UK (COV002), 10178 Brazil (COV003), and 2096 South Africa (COV005)). A total of 11636 participants in COV002 and COV003 met the inclusion criteria for the primary analysis, 5807 received 2 dosesof ChAdOx1-nCoV-19 and 5829 received two doses of control product.

Of the participants in COV002 and COV003 included in the primary analyses, the majority were aged 18-55 years (UK 6542, 87%; Brazil 3676, 90%). Those aged 56 years or older were recruited later and contributed 12% of the total cohort (UK: 1006, 13%, Brazil: 412, 10%). 7045 (61%) participants were female. The majority of participants in the UK were white 6902 (91%). In Brazil 2723 (67%) participants were white..

The timing of priming and booster vaccine administration varied between studies due mainly to delays in manufacturing and shipping. The majority 1459/2741 (53%) of participants in COV002 in the LD/SD group received a second dose at least 12 weeks after the first (median 84 days, IQR 77, 91 days), very few 22/2741 (0.8%) received a second dose in 8 weeks or less. The median time difference for the SD/SD group in COV002 was 69 days (IQR 50, 86 days). Conversely, the majority 2493/4088 (61%) of participants in COV003 in the SD/SD group received a second dose within 6 weeks of the first (median 36 days, IQR, 32, 58 days). (Table below).

Table: Vaccine administration in those included in the primary analysis (The LDSD, SDSD efficacy population as defined in the statistical analysis plan.)

| | COV002 (UK) | | COV003 (Brazil) | |
|---|---|---|---|---|
| Study | ChAdOxl nCoV-19 N (%) | MenACWY N (%) | ChAdOx1 nCoV-19 N (%) | MenACWY N (%) |
| Time between first and second dose LD/SD | | | | |
| < 6 weeks | 0 (0.0%) | 0 (0.0%) | | |
| 6-8 weeks | 10 (0.7%) | 12 (0.9%) | | |
| 9-11 weeks | 624 (45.6%) | 636 (46.3%) | | |
| ≥12 weeks | 733 (53.6%) | 726 (52.8%) | | |
| Time between first and second dose SD/SD | | | | |
| < 6 weeks | 453 (19.1%) | 454 (18.7%) | 1249 (60.5%) | 1244 (61.4%) |
| 6-8 weeks | 517 (21.8%) | 464 (19.1%) | 430 (20.8%) | 431 (21.3%) |
| 9-11 weeks | 595 (25.0%) | 665 (27.4%) | 285 (13.8%) | 275 (13.6%) |
| ≥12 weeks | 812 (34.2%) | 847 (34.9%) | 99 (4.8%) | 75 (3.7%) |

A small proportion of participants were seropositive at baseline (UK: 144, 1.3%, Brazil, 235 2.3%). Three participants seropositive at baseline had subsequent NAAT-positive swabs. One participant had an asymptomatic infection 3 weeks after a first dose of ChAdOx1 nCoV-19. Two other participants in the control group had symptomatic infections 8 and 21 weeks after their baseline sample was taken.

There were 131 cases of symptomatic COVID-19 in LD/SD or SD/SD recipients who were eligible for inclusion in the primary analysis more than 14 days after the second dose of vaccine. There were 30/5807 (0.5%) cases in the vaccine arm and 101/5829 (1.7%) cases in the control group, resulting in vaccine efficacy of 70.4% (95.8% CI 54.8%, 80.6%) (Table 3). In those who received SD/SD vaccines, there were 27/4440 (0.6%) cases in the vaccine arm and 71/4455 (1.6%) cases in the control group, resulting in efficacy of 62.1% (95% CI 41.0%, 75.7%). In those who received a low dose as their first dose of vaccine there were 3/1367 (0.2%) cases in the vaccine group and 30/1374 (2.2%) cases in the control group with vaccine efficacy of 90.0% (95% CI 67.4%, 97.0%) (Table below)

Efficacy against SARS-CoV-2 more than 14 days after a second dose of ChAdOx1 nCoV-19 vaccine in seronegative participants*

| Primary Symptomatic COVID-19 | Total number of cases | ChAdO1l nCoV-19 n/N (%) | Control n/N (%) | VE (CI) 95% unless indicated |
|---|---|---|---|---|
| COV002 (UK) | 86 | 18/3744 (0.5%) | 68/3804 (1.8%) | 73.5% (55.5%, 84.2%) |
| LD/SD recipients | 33 | 3/1367 (0.2%) | 30/1374 (2.2%) | 90.0% (67.4%, 97.0%) |
| SD/SD recipients | 53 | 15/2377 (0.6%) | 38/2430 (1.6%) | 60.3% (28.0%, 78.2%) |
| COV003 (Brazil) | 45 | 12/2063 (0.6%) | 33/2025 (1.6%) | 64.2% (30.7%, 81.5%) |
| All SD/SD recipients | 98 | 27/4440 (0.6%) | 71/4455 (1.6%) | 62.1% (41.0%, 75.7%) |
| All LD/SD and SD/SD recipients# | 131 | 30/5807 (0.5%) | 101/5829 (1.7%) | 70.4% (54.8%, 80.6%) |
| Other symptomatic COVID-19 disease | 18 | 7/5807 (0.1%) | 11/5829 (0.2%) | 36.4% (-63.8%, 75.3%) |
| Any symptomatic COVID-19 disease | 149 | 37/5807 (0.6%) | 112/5829 (1.9%) | 67.1% (52.3%, 77.3%) |
| Asymptomatic or symptoms unknown (COV002) | 69 | 29/3288 (0.88%) | 40/3350 (1.19%) | 27.3% (-17.2%, 54.9%) |
| LD/SD recipients | 24 | 7/1120 (0.62%) | 17/1127 (1.5%) | 58.9% (1.0%, 82.9%) |
| SD/SD recipients | 45 | 22//2168 (1.0%) | 23/2223 (1.0%) | 3.8% (-72.4%, 46.3%) |
| Any NAAT+ | 221 | 68/5807 (1.2%) | 153/5829 (2.6%) | 55.7% (41.1%, 66.7%) |

NAAT+ swab plus at least one of: cough, shortness of breath, fever> 37.8° C., anosmia, ageusia; LD/SD: low dose prime, standard dose boost, SD/SD: two standard dose vaccines given; VE: vaccine efficacy calculated from robust Poisson model. NAAT: nucleic acid amplification test.
*The LDSD, SDSD efficacy population as defined in the statistical analysis plan. 95.8% CI used for primary analysis. VE (CI) calculated from a reduced robust Poisson model which was not adjusted for age

In England and Wales 129529 weekly self-swabs were processed by the DHSC, of which 126324 were able to be matched to study participants. There were 435 positive swabs, of which 354 were able to be matched. Symptoms in these participants were not routinely assessed as swabs were done at home and sent for testing through the post. Asymptomatic infections or those with unreported symptoms were detected in 69 participants. Vaccine efficacy in 24 LD/SD recipients was 58.9% (95% CI 1.0%, 82.9%) and in 45 participants receiving SD/SD vaccine efficacy was 3.8% (95% CI -72.4%, 46.3%). (Table above)

A secondary analysis was conducted of cases occurring more than 21 days after the first standard dose in those who received only standard doses. There were 192 included cases (51/6307 (0.8%) vaccine and 141/6297 (2.2%) control) with a VE of 64.1% (95% CI, 50.5%, 73.9%). (Table below)

Table: Efficacy against SARS-CoV-2 more than 21 days after one or two standard doses of ChAdOx1 nCoV-19 vaccine in seronegative participants*

| | Total number of cases | ChAdOxl nCoV-19 n/N (%) | Control n/N (%) | VE (95% CI) |
|---|---|---|---|---|
| COV002 (UK) | 90 | 28/3060 (0.9%) | 62/3064 (2.0%) | 55.0% (29.7%, 71.1%) |
| COV003 (Brazil) | 102 | 23/3247 (0.7%) | 79/3233 (2.4%) | 71.2% (54.2%, 81.9%) |
| Primary symptomatic COVID-19 | 192 | 51/6307 (0.8%) | 141/6297 (2.2%) | 64.1% (50.5%, 73.9%) |
| Other symptomatic COVID-19 disease | 21 | 12/6307 (0.2%) | 9/6297 (0.1%) | -32.8% (-214.8%, 44.0%) |
| Any symptomatic COVID-19 disease | 213 | 63/6307 (1.0%) | 150/6297 (2.4%) | 58.3% (44.0%, 68.9%) |
| Asymptomatic or symptoms unknown (COV002) | 71 | 34/2751 (1.2%) | 37/2760 (1.3%) | 7.8% (-46.7%, 42.1%) |
| Any NAAT+ | 291 | 102/6307 (1.6%) | 189/6297 (3.0%) | 46.3% (31.8%, 57.8%) |

NAAT+ swab plus at least one of: cough, shortness of breath, fever> 37.8° C., anosmia, ageusia; VE: vaccine efficacy calculated from robust Poisson model. NAAT: nucleic acid amplification test. *The 1 dose SD efficacy population as defined in the statistical analysis plan. SD: standard dose vaccine. £ VE (CI) calculated from a reduced robust Poisson model (excluding the age group category due to the full model failing to converge). Participants with a low dose prime were excluded.

During the time period more than 21 days after the first dose 10 participants were hospitalised due to COVID-19 (defined as WHO clinical progression score >= 4), of which, 2 cases were assessed as severe (WHO score >= 6) including one fatal case. All 10 cases were in the control group. (Table below).

Table: Hospitalisation and severe disease

| | ChAdOx1 nCoV-19 | Control |
|---|---|---|
| Hospitalisation (WHO clinical progression score of 4 or more) | | |
| <= 21 days after the first dose | 2* | 6 |
| > 21 days post first dose, <= 14 days post second dose | 0 | 5 |
| > 14 days post second dose | 0 | 5 |
| Severe COVID-19 (WHO clinical progression score of 6 or more) | | |
| <= 21 days after the first dose | 0 | 0 |
| > 21 days post first dose, <= 14 days post second dose | 0 | 1 |
| > 14 days post second dose | 0 | 1 |

VE not calculated due to small number for analysis. One case of severe disease was fatal. WHO clinical progression scores were assigned by an independent blinded endpoint adjudication committee. * one case on day of vaccination and one case ten days after vaccination.

Five cases included in the primary analysis occurred in those who were more than 55 years old. Vaccine efficacy in older age groups could not be assessed but will be determined after more cases have accrued.

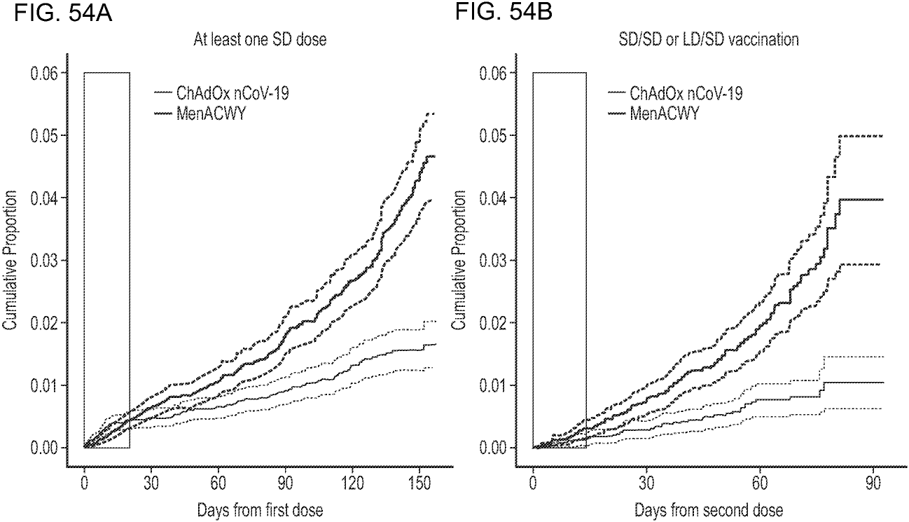

We refer also to FIG. 54, which shows Kaplan Meier cumulative incidence of symptomatic NAAT+ COVID-19 disease after 1 or more standard doses (left) or after 2 doses(right). Grey shaded area shows the exclusion period after each dose in which cases were excluded from the analysis. Dotted lines show 95% confidence region.

### Discussion of Example 22

No previous trials have reported vaccine efficacy of a viral vectored coronavirus vaccine and so we provide the first evidence that induction of immune responses against spike protein using viral vectors provides protection against the disease in humans.

In those who received two standard doses, efficacy against primary symptomatic COVID-19 was consistent in both the UK and Brazil populations, with 60% and 64% efficacy respectively. Efficacy of 90% seen in those who received a low dose as prime in the UK was surprisingly high. A similar contrast in efficacy between the LD/SD and SD/SD recipients with asymptomatic infections provides support for the observation. Use of a low dose for priming, could provide substantially more vaccine for distribution at a time of constrained supply, and these data imply that this would not compromise protection. While a vaccine that could prevent COVID-19 disease would have a substantial public health benefit, prevention of asymptomatic infection could reduce viral transmission and protect those with underlying health conditions who do not respond to vaccination, those who cannot be vaccinated, and those who do not have access to a vaccine, providing wider benefit. Here we show that efficacy against asymptomatic infection following ChAdOx1 nCoV-19 is 59% in those who received a low-dose prime and 4% in those who received two standard doses.

Some regulatory authorities consider that the lower bound of the confidence interval for efficacy should be higher than 20%, with other authorities more stringent and anticipating a lower bound of 30% for licensure (Center for Biologics Evaluation and Research. Development and Licensure of Vaccines to Prevent COVID-19. 2020 (8th November 2020)). Here we present data which exceed both these thresholds.

### Example 23. Exposure to AZD1222: Protection After the First Vaccine Dose and Effect of Dose Interval on VE ≥ 15 Days after Second Dose

### Exposure to AZD1222

12021 participants of the 4 studies included in the application have received at least one dose of AZD1222. Of these participants, 8266 (68.8%) have received 2 doses of AZD1222 (see Table below). Overall and in the primary efficacy analysis set, approximately one-third of participants each had a dose interval in the range of < 6 weeks, 6 to 11 weeks, or ≥ 12 weeks.

| Parameter | | Any Dose for Safety Analysis Set | | SDSD + LDSD Seronegative for Efficacy Analysis Set | |
|---|---|---|---|---|---|
| | | A2D12Z2 (N = 12021) | Control (N = 11724) | AZD1222 (N = 5807) | Control (N = 5829) |
| Dose level², a(%) | LDSD | 1516 (12.6) | 1472(12.8) | 1367 (23.5) | 1374(23.6) |
| | LDLD | 127 (1.1) | 69 (0.6) | 0 | 0 |
| | SDSD | 6568 (54.6) | 6472 (55.2) | 4440 (26.5) | 4455 (76.4) |
| | SDLD | 55 (0.5) | 36 (0.3) | 0 | 0 |
| | LD | 105 (2.5) | 281 (2.4) | 0 | 0 |
| | SD | 3450 (28.7) | 3394 (28.9) | 0 | 0 |
| | Total | 12021 | 11724 | 5807 | 5829 |
| Dose interval, n(%) | < 6 weeks | 3412 (41.3) | 3234 (40.2) | 1702 (29.3) | 1698 (29.1) |
| | 6-8 weeks | 680 (8.2) | 608 (2.5) | 508 (9.8) | 527(9.0) |
| | 9-11 weeks | 1558 (18.8) | 1550 (19.3) | 1444 (24.9) | 1488 (25.5) |
| | 2-12 weeks | 2616 (31.6) | 1681 (33.1) | 2093 (36.0) | 2116 (36.3) |
| | Total | 8266 | 8049 | 5507 | 5829 |

* Dose level of control group is decided by the dose level of corresponding vaccine group.
Total row includes the number of participents with non-missing data for the corresponding characteristic and was used as the denominator for calculating percentages for all catagories.

### Protection After the First Vaccine Dose

Exploratory analyses were conducted to investigate whether protective immunity was induced by the first dose and what the duration of protection was. The follow-up time began at 22 days after the first dose and participants were censored from the analysis at the earliest time point of when they received a second dose or at 12 weeks post-dose 1. Results (see Table below) indicated that the first dose provides protective immunity at least until 12 weeks.

| | Participants with events | | | | VE (%) | 95% CI |
|---|---|---|---|---|---|---|
| | AZD1222 | | Control | | | |
| | N | n (%) | N | n (%) | | |
| Cases to Week 12 | 7998 | 12 (0.15) | 7982 | 44 (0.55) | 73.00 | (48.79, 85.76) |

Table: Vaccine efficacy for incidence of first SARS-CoV-2 virologically-confirmed COVID-19 occurring post first dose + 22 Days and before second dose of vaccine or 12 weeks post dose 1 5807 participants in the AZD1222 group for the SDSD + LDSD Seronegative for Efficacy Analysis Set had a median duration of follow-up from 15 days post second dose (i.e., endpoint for primary efficacy endpoint) of 48.0 days (range, 1 to 79 days) and from first dose of 132.0 days (range, 41 to 158 days); 5829 participants in the control group had a median duration of follow-up from 15 days post second dose of 48.0 days (range, 1 to 79 days) and from first dose of 133.0 days (range, 35 to 158 days).

### Effect of Dose Interval on VE ≥ 15 Days After Dose 2

The dataset in which efficacy of a two-dose regimen has been demonstrated contained data over a wide range of dose intervals (4 to 26 weeks): 29.3% were < 6 weeks, 34.7% were 6-11 weeks, and 36.0% were ≥ 12 weeks.

Subgroup analyses were conducted of vaccine efficacy by dosing interval. In line with immunogenicity data where increases in the binding and neutralising antibody responses were observed with increased dosing interval, efficacy was demonstrated with more certainty for dose intervals from 8-12 weeks. For the subgroup with dosing interval 8-11 weeks, VE was 72.85%, 95% CI (43.45, 86.97), for the subgroup with dosing interval > 11 weeks, it was 81.90%, 95% CI (59.93, 91.90). Exploratory subgroup analyses showed vaccine efficacy around 80% for dosing intervals longer than 11 weeks, but data were limited and estimates were associated with wide confidence intervals.

Although illustrative embodiments of the invention have been disclosed in detail herein, with reference to the accompanying drawings, it is understood that the invention is not limited to those precise embodiments and that various changes and modifications can be effected therein by one skilled in the art without departing from the scope of the invention as defined by the appended claims and their equivalents.

Table of sequences

| | |
|---|---|
| SEQ ID NO: 1 | Amino acid sequence of spike protein of SARS-CoV2 (nCoV-19) |
| SEQ ID NO: 2 | ChAdOx2: Viral vector based on Chimpanzee adenovirus C68 |
| SEQ ID NO: 3 | nucleotide sequence codon optimised and revised to eliminate repeat bases - encoding spike protein without tPA leader |
| SEQ ID NO: 4 | nucleotide sequence codon optimised and revised to eliminate repeat bases - encoding spike protein with tPA leader |
| SEQ ID NO: 5 | tPA amino acid sequence (P->A mutant) |
| SEQ ID NO: 6 | tPA amino acid sequence (naturally occurring P) |
| SEQ ID NO: 7 | tPA amino acid sequence SEQ ID NO: 5 without ‘RR’ |
| SEQ ID NO: 8 | tPA amino acid sequence SEQ ID NO: 6 without ‘RR’ |
| SEQ ID NO: 9 | nucleotide sequence encoding tPA SEQ ID NO: 5, which has been codon optimised for human codon usage |
| SEQ ID NO: 10 | Amino acid sequence of tPA-Spike fusion (tPA underlined) (i.e. amino acid sequence encoded by SEQ ID NO: 4) |
| SEQ ID NO: 11 | Nucleotide sequence from SARS-CoV2 (nCoV 19) genome for spike protein (From Accession number MG772933.1) |
| SEQ ID NO: 12 | Amino acid sequence of tPA-spike receptor binding domain |
| SEQ ID NO: 13 | Nucleotide sequence of tPA-spike receptor binding domain |
| SEQ ID NO: 14 | Amino acid sequence spike prefusion protein with tPA |
| SEQ ID NO: 15 | Nucleotide sequence spike prefusion protein with tPA |

1. A composition comprising a viral vector, the viral vector comprising nucleic acid having a polynucleotide sequence encoding the spike protein from the coronavirus SARS-CoV2, wherein the viral vector is an adenovirus based vector.

2. A composition according to claim 1, wherein the adenovirus based vector is ChAdOx 1.

3. A composition according to claim 1, wherein the spike protein comprises the receptor binding domains (RBDs).

4. A composition according to claim 1, wherein the spike protein is full length spike protein.

5. A composition according to claim 1, wherein the spike protein is present as a fusion with the tissue plasminogen activator (tPA) sequence in the order N-terminus - tPA - spike protein -C-terminus.

6. A composition according to claim 5, wherein the tPA comprises an amino acid sequence selected from the group consisting of SEQ ID NO: 5, SEQ ID NO: 6, SEQ ID NO: 7, and SEQ ID NO: 8.

7. A composition according to claim 1, wherein the spike protein comprises the amino acid sequence SEQ ID NO: 1.

8. A composition according to claim 1, wherein the polynucleotide sequence comprises an amino acid sequence selected from the group consisting of SEQ ID NO: 3 and SEQ ID NO: 4.

9. A composition according to claim 2, wherein the viral vector sequence comprises a sequence with ECACC accession number 12052403.

10. A composition according to claim 1, wherein the composition is configured such that administration of a single dose of the composition to a mammalian subject induces protective immunity in the subject.

11. A composition according to claim 1, wherein the composition is configured such that administration of a first dose of the composition to a mammalian subject followed by administration of a second dose of the composition to the mammalian subject induces protective immunity in the subject.

12. A composition according to claim 1, wherein the composition is configured for use in induction of an immune response against SARS-CoV2 in a mammalian subject.

13. A composition according to claim 1, wherein the composition is configured for use in preventing SARS-CoV2 infection in a mammalian subject.

14. A medicament for prevention of SARS-CoV2 infection in a mammalian subject comprising a composition according to claim 1.

15. A method of inducing an immune response against SARS-CoV2 in a mammalian subject, the method comprising administering a dose of a composition according to claim 1 to the subject.

16. A method of using the composition of claim 12 or claim 13 comprising the steps of:
administering a first dose of the composition to a subject; and
administering a second dose of the composition to the subject,
wherein the first dose and the second dose each comprise about the same number of viral particles.

17. The method of claim 15, wherein the first and second dose each comprise about 5 x 10¹⁰ viral particles.

18. The method of claim 16, wherein the second dose is administered at an interval of less than 6 weeks, 6 to 8 weeks, 9 to 11 weeks, or at least 12 weeks after administration of the first dose.

19. The method of claim 16, wherein the composition is administered by a route of administration selected from a group consisting of intranasal, aerosol, intradermal and intramuscular.
